(19) United States
(12) Patent Application Publication
(10) Pub. No.: US 2024/0115671 A1
(43) Pub. Date: Apr. 11, 2024

(54) INTRACELLULAR DELIVERY OF THERAPEUTIC PROTEINS DESIGNED TO INVADE AND AUTONOMOUSLY LYSE AND METHODS OF USE THEREOF

(71) Applicants: University of Massachusetts, Boston, MA (US); Ernest Pharmaceuticals, Inc., Hadley, MA (US)

(72) Inventors: Neil S. Forbes, Hadley, MA (US); Vishnu Raman, Sunderland, MA (US); Nele Van Dessel, Easthampton, MA (US); Jeanne A. Hardy, Amherst, MA (US)

(21) Appl. No.: 18/276,628

(22) PCT Filed: Feb. 9, 2022
(86) PCT No.: PCT/US2022/070582

§ 371 Date: Aug. 9, 2023

Related U.S. Application Data
(60) Provisional application No. 63/147,506, filed on Feb. 9, 2021.

(51) Int. Cl. A61K 38/48; A61K 35/74; A61K 38/46; C12N 1/20; C12N 1/36; C12N 9/16; C12N 9/64; C12N 15/74

(52) U.S. Cl. CPC A61K 38/4873; A61K 35/74; A61K 38/465; C12N 1/20; C12N 1/36; C12N 9/16; C12N 9/6472; C12N 15/74; C12Y 301/04; C12Y 304/22056; C12N 2800/101; C12N 2830/002

(57) ABSTRACT

Provided herein is a bacterial delivery platform that harnesses mechanisms unique to Salmonella to intracellularly deliver protein-based drugs.

### PRIORITY

This application claims the benefit of priority of U.S. Provisional Patent Application No. 63/147,506, filed on Feb. 9, 2021, the benefit of priority of which is claimed hereby, and which is incorporated by reference herein in its entirety.

### GOVERNMENT SUPPORT

This invention was made with government support under grant no. R43 CA233136 awarded by The National Institutes of Health. The government has certain rights in the invention.

### BACKGROUND OF THE INVENTION

Cancer is generally characterized by an uncontrolled and invasive growth of cells. These cells may spread to other parts of the body (metastasis). Conventional anticancer therapies, consisting of surgical resection, radiotherapy and chemotherapy, can be effective for some cancers/patients; however, they are not effective for many cancer sufferers. Thus, further medical treatments are needed.

The role of bacteria as an anticancer agent has been recognized for over 100 years, and many genera of bacteria, including Clostridium, Bifidus, and Salmonella, have been shown to preferentially accumulate in tumor tissue and cause regression.

The use of Salmonella typhimurium to treat solid tumors began with the development of a nonpathogenic strain, VNP20009. Well-tolerated in mice and humans, this strain has been shown to preferentially accumulate (>2000-fold) in tumors over the liver, spleen, lung, heart and skin, retarding tumor growth between 38-79%, and prolonging survival of tumor-bearing mice. In initial clinical trials, S. typhimurium was found to be tolerated at high dose and able to effectively colonize human tumors.

### SUMMARY OF THE INVENTION

Engineered, non-pathogenic Salmonella selectively colonize tumors one thousand-fold more than any other organ, invade and deliver therapies cytosolically into cancer cells making the bacteria ideal delivery vehicles for cancer therapy. It is herein demonstrated that controlling the activity of flhDC and subsequent flagellar expression in engineered Salmonella enables intracellular protein delivery selectively in tumor cells in vivo and in vitro. The expression of flhDC/flagella is controlled to enable both colonization of tumors and invasion into cancer cells for the purposes of intracellular protein and therapeutic delivery. Flagella are needed for cell invasion into cancer cells in vitro and in vivo. However, flagellar expression of Salmonella in the bloodstream and/or in systemic circulation causes rapid clearance and significantly reduces tumor colonization. As a result, an inducible version of flhDC was genetically engineered into an engineered strain of Salmonella lacking a native version of the transcription factor (alternatively, the endogenous promoter for flhDC can replaced with an inducible promoter). The inducible system allowed for tight expression control of flhDC within the therapeutic strain. Salmonella lacking the ability to express flhDC colonized tumors with greater selectivity than a parental control strain. Inducing expression of flhDC by administration of ‘remote controlling’, with, for example, arabinose in an arabinose inducible system, within intratumoral, engineered Salmonella enabled intracellular invasion and protein delivery into tumor cells.

Herein is described Salmonella containing and method to control flagellar expression through external means (e.g., a small molecule inducible genetic circuit or inducible expression system) in such a way that engineered strains of Salmonella do not express flagellin systemically. Once the bacteria have colonized tumors to optimal levels, then a ‘remote control’/inducible strategy is employed where a small molecule is used to induce expression of flagella and the type 3 secretion system by activating expression of a recombinant and/or inducible version of the motility regulator, flhDC.

Another aspect provides for the deletion of the SseJ gene in a Salmonella delivery strain. This gene constricts the location of the Salmonella to the Salmonella-containing vacuole (SCV), increasing the delivery potential of the strain. This can be in combination with/without the previously described control of delivery.

One aspect provides a bacterial cell comprising: a) inducible expression of flagella; and b) a lysis gene or lysis cassette operably linked to an intracellularly induced Salmonella promoter. In one aspect, the bacterial cell is an intratumoral bacteria cell. In another aspect, the bacterial cell is a Clostridium, Bifidus, E. coli or Salmonella cell. In one aspect, bacterial cell is a Salmonella cell. In one aspect, the lysis cassette is Lysin E from phage phiX174, the lysis cassette of phage iEPS5, or the lysis cassette from lambda phage. In another aspect, intracellularly induced Salmonella promoter is a promoter for one of the genes in Salmonella pathogenicity island 2 type III secretion system (SPI2-T3SS) selected from the group SpiC/SsaB, SseF, SseG, SseI, SseJ, SseK1, SseK2, SifA, SifB, PipB, PipB2, SopD2, GogB, SseL, SteC, SspH1, SspH2, or SirP.

In one aspect, the cell does not comprise endogenous flhDC expression. In another aspect, the cell comprises an exogenous inducible promoter operably linked to an endogenous or exogenous flhDC gene. In one aspect, the exogenous inducible promoter is operably linked to the endogenous flhDC gene. In another aspect, the exogenous inducible promoter is operably linked the exogenous flhDC gene. In aspect, the exogenous inducible promoter comprises the arabinose inducible promoter PBAD (L-arabinose), LacI (IPTG), salR, or nahR (acetyl salicylic acid (ASA)).

In one aspect, the bacterial cell comprises a SseJ deletion or wherein expression of SseJ has been reduced.

One aspect provides a cell comprises a plasmid that expresses a peptide. In one aspect, the peptide is a therapeutic peptide, such as NIPP1 or activated caspase 3.

One aspect provides a composition comprising a population of cells described herein and a pharmaceutically acceptable carrier.

Another aspect provides a method to selectively colonize cancer cells, such as a tumor and/or tumor associated cells comprising administering a population of the bacterial cells described herein to a subject in need thereof. In one aspect, the tumor associated cells are tumor cells or intratumoral immune cells, cancer cells or stromal cells within tumors. Another aspect provides a method to treat cancer comprising administering to a subject in need thereof an effective amount of a population of the bacterial cells described herein to treat said cancer. A further aspect provides a method of inhibiting tumor growth/proliferation or reducing the volume/size of a tumor comprising administering to a subject in need thereof an effective amount of a population of the bacterial cells described herein, so as to suppress tumor growth or reduce the volume of the tumor. Another aspect provides a method to treat, reduce formation/number or inhibit spread of metastases comprising administering to subject in need thereof an effective amount of a population of the bacterial cells described herein, so as to treat, reduce formation/number or inhibit spread of metastases. In one aspect, the tumor, tumor associated cells, cancer, or metastases are a lung, liver, kidney, breast, prostate, pancreatic, colon, head and neck, ovarian and/or gastroenterological tumor, tumor associated cells, cancer or metastases. In one aspect, the bacterial cells deliver a therapeutic peptide to said tumor, tumor associated cells, cancer or metastases. In one aspect, the peptide is NIPP1 or activated caspase 3. In one aspect, the cells do not express endogenous flhDC. In another aspect, expression of flhDC in the bacterial cell is under the control of an inducible promoter, wherein the bacterial cells comprise an exogenous inducible promoter controlling expression of endogenous flhDC or the bacterial cells comprise an exogenous inducible promoter operably linked an exogenous flhDC gene. In one aspect, the expression of flhDC is induced after said tumor, tumor associated cells, cancer or metastases have been colonized (e.g., between 1×10⁶ and 1×10¹⁰ CFU/g tumor) by said bacteria.

One aspect provides a bacterial cell comprising: a) a SseJ deletion or wherein expression of SseJ has been reduced; and b) a lysis gene or lysis cassette operably linked to an intracellularly induced Salmonella promoter. In one aspect, the bacterial cell is an intratumoral bacteria cell. In another aspect, the bacterial cell is a Clostridium, Bifidus or Salmonella cell. In aspect, the bacterial cell is a Salmonella cell. In one aspect, the lysis cassette is Lysin E from phage phiX174, the lysis cassette of phage iEPS5, or the lysis cassette from lambda phage. In another aspect, the intracellularly induced Salmonella promoter is a promoter for one of the genes in Salmonella pathogenicity island 2 type III secretion system (SPI2-T3SS) selected from the group SpiC/SsaB, SseF, SseG, SseI, SseJ, SseK1, SseK2, SifA, SifB, PipB, PipB2, SopD2, GogB, SseI SteC, SspH1, SspH2, or SirP.

In one aspect, the cell of any one of claims 28-33, wherein the cell does not comprise endogenous flhDC expression. In another aspect, the cell comprises an exogenous inducible promoter operably linked to an endogenous or exogenous flhDC gene. In another aspect, the exogenous inducible promoter is operably linked to the endogenous flhDC gene. In another aspect, the exogenous inducible promoter is operably linked the exogenous flhDC gene. In aspect, the exogenous inducible promoter comprises the arabinose inducible promoter PBAD (L-arabinose), LacI (IPTG), nahR (acetyl salicylic acid (ASA)), or salR acetyl salicylic acid (ASA).

In one aspect, the bacterial cell comprises a plasmid that expresses a peptide. In one aspect, the peptide is a therapeutic peptide, such as NIPP1 or activated caspase 3.

One aspect provides for a composition comprising a population of cells as described herein and a pharmaceutically acceptable carrier.

One aspect provides a method to colonize a tumor and/or tumor associated cells comprising administering a population of the bacterial cells described herein to a subject in need thereof. In one aspect, the tumor associated cells are tumor cells, intratumoral immune cells or stromal cells within tumors. In one aspect there is provided a method to treat cancer comprising administering to subject in need thereof an effective amount of a population of the bacterial cells described herein so as to treat said cancer. Another aspect provides a method of inhibiting tumor growth/proliferation or reducing the volume/size of a tumor comprising administering to subject in need thereof an effective amount of a population of the bacterial cells described herein, so as to suppress tumor growth or reduce the volume of the tumor. A further aspect provides a method to treat, reduce formation/number or inhibit spread of metastases comprising administering to subject in need thereof an effective amount of a population of the bacterial cells described herein, so as to treat, reduce formation/number or inhibit spread of metastases. In one aspect, the tumor, tumor associated cells, cancer, or metastases are a lung, liver, kidney, breast, prostate, pancreatic, colon, head and neck, ovarian and/or gastroenterological tumor, tumor associated cells, cancer or metastases. In another aspect, the bacterial cells deliver a therapeutic peptide, such as NIPP1 or activated caspase 3, to said tumor, tumor associated cells, cancer or metastases. In one embodiment, endogenous expression of flhDC is under control of an exogenous inducible promoter. In another aspect, expression of flhDC is under the control of an inducible promoter, wherein the bacterial cells comprise an exogenous inducible promoter operably linked an exogenous flhDC gene. In a further aspect, the expression of flhDC is induced after said tumor, tumor associated cells, cancer or metastases have been colonized by said bacteria.

One aspect provides a bacterial cell comprising: a) constitutive or inducible expression of a therapeutic peptide, wherein the therapeutic peptide is activated caspase-3 and wherein said activated caspase-3 is expressed as an activated protein without further processing; and b) a lysis gene or lysis cassette operably linked to an intracellularly induced Salmonella promoter. In one aspect, the bacterial cell is an intratumoral bacteria cell. In one aspect, the bacterial cell is a Clostridium, Bifidus or Salmonella cell. In another aspect, the bacterial cell is a Salmonella cell. In one aspect, the lysis cassette is Lysin E from phage phiX174, the lysis cassette of phage iEPS5, or the lysis cassette from lambda phage. In one aspect, the intracellularly induced Salmonella promoter is a promoter for one of the genes in Salmonella pathogenicity island 2 type III secretion system (SPI2-T3SS) selected from the group SpiC/SsaB, SseF, SseG, SseI, SseJ, SseK1, SseK2, SifA, SifB, PipB, PipB2, SopD2, GogB, SseL, SteC, SspH1, SspH2, or SirP.

In another aspect, the bacterial cell does not comprise endogenous flhDC expression. In one aspect, the bacterial cell comprises an exogenous inducible promoter operably linked to an endogenous or exogenous flhDC gene. In one aspect, the exogenous inducible promoter is operably linked to the endogenous flhDC gene. In another aspect, the exogenous inducible promoter is operably linked the exogenous flhDC gene. In one aspect, the exogenous inducible promoter comprises the arabinose inducible promoter PBAD (L-arabinose), LacI (IPTG), nahR (acetyl salicylic acid (ASA)) or salR acetyl salicylic acid (ASA).

In aspect, the bacterial cell comprises a SseJ deletion or wherein expression of SseJ has been reduced.

One aspect provides for cells that express at least one additional exogenous therapeutic peptide, such as NIPP1.

Another aspect provides a composition comprising a population of cells described herein and a pharmaceutically acceptable carrier.

One aspect provides a method to colonize a tumor and/or tumor associated cells comprising administering a population of the bacterial cells described herein to a subject in need thereof. In one aspect, the tumor associated cells are tumor cells, intratumoral immune cells or stromal cells within tumors. One aspect provides a method to treat cancer comprising administering to subject in need thereof an effective amount of a population of the bacterial cells described herein so as to treat said cancer. In one aspect there is provided a method of inhibiting tumor growth/proliferation or reducing the volume/size of a tumor comprising administering to subject in need thereof an effective amount of a population of the bacterial cells of any one of claims described herein, so as to suppress tumor growth or reduce the volume of the tumor. One aspect provides a method to treat, reduce formation/number or inhibit spread of metastases comprising administering to subject in need thereof an effective amount of a population of the bacterial cells described herein, so as to treat, reduce formation/number or inhibit spread of metastases. In one aspect, the tumor, tumor associated cells, cancer, or metastases are a lung, liver, kidney, breast, prostate, pancreatic, colon, head and neck, ovarian and/or gastroenterological tumor, tumor associated cells, cancer or metastases. In one aspect, the bacterial cells deliver said caspase to said tumor, tumor associated cells, cancer or metastases. In another aspect, the bacterial cells deliver at least one additional exogenous therapeutic peptide, such as NIPP1. In aspect, the endogenous expression of flhDC is under control of an exogenous inducible promoter. In another aspect, the expression of flhDC is under the control of an inducible promoter, wherein the bacterial cells comprise an exogenous inducible promoter operably linked an exogenous flhDC gene. In one aspect, the bacterial cells do not express endogenous flhDC. In one aspect, the expression of flhDC is induced after said tumor, tumor associated cells, cancer or metastases have been colonized by said bacteria.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-G: Intracellular lifestyle of Salmonella is controlled by flhDC. A) The design goals were to genetically engineer a bacterial vehicle that (1) synthesizes (makes) a protein drug (yellow/purple), (2) actively invades into cancer cells and (3) releases the drug. With time, drugs escape Salmonella vacuoles (SCVs, red). B) Salmonella (light blue, arrows) invade cancer cells (red). C) Seventy percent of Salmonella (red; white arrow) were intracellular (co-localized red and green; black arrows) within tumors in vivo (***, P<0.001). D) In tumors in mice, Salmonella invaded multiple cell types, including immune, carcinoma (epithelial) and other associated (stromal) cells. E) In cancer cells in monolayer, flhDC re-expression (flhDC+) increased invasion (black arrows) compared non-expressing controls (flhDC−; ***, P<0.001). F) In a three-dimensional tumor-on-a-chip, flhDC+Salmonella, with a green intracellular reporter, invaded more than flhDC− controls (**, P<0.01). G) After administration to tumor-bearing mice, re-expression of flhDC increased invasion into cancerous and immune cells (*, P<0.05).

FIGS. 2A-J. Design of ID Salmonella to release protein into cells. A) Salmonella with either the PsifA-GFP or PsseJ-GFP reporter constructs expressed GFP after invasion (white arrows). Extracellular expression (black arrows) from PsseJ-GFP was less than PsifA-GFP (***, P<0.001). The intracellular activity of the PsseJ promoter was four times greater than extracellular activity (***, P<0.001). B) Induction of PBAD-LysE at 96 h (arrow) induced bacteria lysis at a rate of 0.39 hr⁻¹. C) When administered to MCF7 cancer cells, 68% of intracellular Salmonella with PsseJ-lysE lysed, significantly more than PsseJ-GFP controls (***, P<0.001). C) Salmonella with PsseJ-lysE and Plac-GFP delivered GFP into the cellular cytoplasm. Only released, and not intra-bacterial, GFP was stained. E) Intracellular ID Salmonella lysed at a rate of 0.33 hr⁻¹ (half-life=2.1 h). F) In liquid culture, PBAD-lysE and PsseJ-lysE Salmonella grew at similar rates as non-transformed controls (white bars). When intracellular, PsseJ-lysE Salmonella, lysed at a similar rate as induced PBAD-lysE Salmonella in culture (black bars). G) Bacterial EGFP production per colony forming unit (CFU). H) After invasion and before lysis, Salmonella (light blue, white arrow) were in LAMP1-stained SCVs (red, yellow arrows). After lysis, GFP (green, black arrow) remained within the membranes of SCVs. From 6 to 24 h after invasion, the percentage of released GFP in the cytosol, and not SCVs, increased from 25% to 75% (***, P<0.001). I) In phalloidin-stained cancer cells (red) released GFP (green, black arrows) moved from SCVs near the nucleus (blue) to throughout the cytoplasm. J) ID Salmonella (left) lyse and GFP diffuses through the cytosol of a cancer cells (right). Temporal profiles of GFP intensity, centered on the lysed bacteria.

FIGS. 3A-G. PsseJ and flhDC are components of ID Salmonella delivery to tumors. A) Most released GFP (green, black arrow) originated from lysed Salmonella in LAMP1-stained SCVs (red, yellow arrow). Cytosolic bacteria (light blue, white arrow) did not lyse (***, P<0.001) or release GFP. Only released GFP was stained. B) Predominantly cytoplasmic ΔsifA remained intact (red, white arrows) and had less lysis (green, black arrows) than predominantly vacuolar ΔsseJ and ID Salmonella (***<P<0.001). C) GFP (green, arrows) was only delivered when Salmonella was transformed with both PBAD-flhDC and PsseJ-LysE (***, P<0.001). D) After injection of 2×10⁶ bacteria/mouse to BALB/c mice with 4T1 tumors, ID Salmonella delivered GFP into cancer cells (arrows). E) Delivered GFP was present in extracts from tumors (T), but not livers (L) or spleens (S). F) Administration of ID Salmonella with induced PBAD-flhDC to BALB/c mice with 4T1 tumors delivered GFP (arrows) to more cells than flhDC− controls (***, P<0.001). G) Luciferase-expressing ID Salmonella were intravenously injected into BALB/c mice with 4T1 tumors and bacterial density in tumors was measured for 14 days with bioluminescence imaging.

FIGS. 4A-E. Efficacy of ID Salmonella. A) Anti-actin nanobody (NB) and GFP (Ctr) was delivered into 4T1 cancer cells with ID Salmonella. Beta-actin was immuno-precipitated with delivered nanobody and was enriched 2.5 times compared to controls. B) ID Salmonella delivery of NIPP1-CD and CT Casp-3 caused more death (red, white arrows) in Hepa 1-6 cells compared to controls (***, P<0.001, top). Cells invaded with control Salmonella (green, black arrows) or not invaded (yellow arrows) did not die. C) Delivery of NIPP1-CD and CT Casp 3 caused cell death (red) in microfluidic tumor masses (*, P<0.05; **, P<0.01). Death increased with time as Salmonella invaded into cells and delivered protein (*, P<0.05). D) Delivery of CT Casp-3 decreased growth of 4T1 mammary tumors compared to bacterial controls that delivered GFP (*, P<0.05; n=3). E) Nineteen days after injection, the volume of CT-Casp-3-treated Hepa 1-6 liver tumors were 12% of controls (***, P<0.001; n=3; left). Treatment with CT Casp-3 reduced tumor growth rate compared to Salmonella controls (P<0.05, middle), significantly increased survival (P<0.05, right) and cured one mouse.

FIGS. 5A-D. Tumor selectivity of ΔflhD and ΔsifA Salmonella. A) Tumor colonization of ΔflhD Salmonella was unchanged as compared to the parental control. However, liver colonization of ΔflhD Salmonella was ten-fold less than control (*, P<0.05). B) Although not statistically significant, the colonization levels of all three flhDC overexpressing tumors were less than those of the parental control (P=0.34). C) The aflagellate, flhDC expressing ΔfliGHI Salmonella colonized the livers eight-fold and twelve-fold more than ΔflhD and ΔfliGHI+ΔflhD strains, respectively (*, P<0.05) D) ΔfliGHI, ΔflhD, ΔfliGHI+ΔflhD Salmonella did not differ in tumor colonization levels.

FIGS. 6A-I. flhDC activity is needed for increased bacterial dispersion in tumors. A) Mice bearing 4T1 tumors were injected with ΔflhD Salmonella. 48 hours after bacterial injection, half of the mice were administered with arabinose 48 and 72 hours after bacterial injection in order to induce flhDC expression. B)flhDC uninduced Salmonella were non-motile and formed distinctly separated colonies either in necrotic (yellow arrows) or viable tissue (green arrows). C) 75% of distinct colonies resided in necrosis while only 25% of colonies were located in viable tumor tissue (**, P<0.01). D) The growth rate of bacteria in necrosis (0.12 hr-1) was marginally higher than those in viable tumor (0.11 hr-1) tissue (*, P<0.05) which corresponded to doubling times of (E) 6 hours in necrosis versus 6.5 hours in viable tumor tissue (*, P<0.05). F) Dense bacterial colony sizes (red borders) were visibly larger with flhDC induced as compared to uninduced tumors. Scale bar is um. G) Dense colony sizes were 50% larger within tumors treated with flhDC induced as opposed to uninduced tumors (*, P<0.05). H) The abundance of satellite colonies (green arrows) outside of main dense bacterial colonies was visually greater in tumors containing flhDC induced as opposed to uninduced Salmonella. Scale bar is 200 um. I) There was a two-fold greater abundance of isolated satellite colonies in tumors containing flhDC induced as opposed to uninduced Salmonella (*, P<0.05).

FIGS. 7A-I. flhDC activity increases the dispersion of intracellular Salmonella within tumors in vitro and in vivo. A) A microfluidic tumor-on-a-chip was infected with either flhDC induced or uninduced IR Salmonella. These bacteria expressed GFP selectively inside cells. B) flhDC induced Salmonella (green) were distributed throughout tumor masses while uninduced bacteria were faintly detectable towards the front edge of the tumor mass (white arrows). Scale bar is 100 um. C) The amount of intracellular bacteria was 50-fold to 75-fold greater for x>0.5 in tumors with flhDC induced as opposed to uninduced Salmonella (**, P<0.01; ***, P<0.001). D) The amount of flhDC induced, intracellular bacteria continued to increase over time as compared to the uninduced control (*, P<0.05; **, P<0.01; ***, P<0.001). E) Mice were infected with IR Salmonella and one group was administered with arabinose to express flhDC. F) Dense uninduced Salmonella contained significantly less intracellular bacteria (yellow arrows) as compared with induced colonies (yellow borders). G) The fraction of intracellular flhDC induced Salmonella was three-fold greater than uninduced colonies within tumors (*, P<0.05). H) The dispersion of intracellular bacteria in tumors was greater after flhDC induction. Euclidean distance mapping of intracellular bacteria showed that tumor coverage was (I) 50% greater when flhDC was expressed (*, P<0.05).

FIGS. 8A-B. flhDC expression was needed for intracellular protein delivery into broadly distributed cells within tumors in vivo. A) When flhDC was induced, Intracellular delivery occurred in spatially more distributed cells within tumors (white arrows). Euclidean distance mapping demonstrated that tumors treated with flhDC induced Salmonella had cells with GFP delivery that were (B) 60% more spatially distributed within tumors as compared to the uninduced control (*, P<0.05).

FIGS. 9A-D. Engineered Salmonella are more effective for intracellular delivery than cytosolic Salmonella. A) The ΔsifA Salmonella colonized tumors ten-fold less than the parental control strain (*, P<0.05). B) The ΔsifA Salmonella colonized the liver 15-fold less than the parental control strain (*, P<0.05). C) Cytosolic ΔsifA Salmonella remained almost exclusively intact (red) within cancer cells while the majority of FID Sal lysed within cancer cells (green dots FID Sal panel). D) FID Sal lysed at least 18-fold more than ΔsifA Salmonella at any point in time (***, P<0.001).

FIGS. 10A-J. flhDC activity decreases activity by enabling vacuolar escape of Salmonella. A) 4T1 cells in monolayer were infected with either ID Sal or FID Sal. B) While overall bacterial invasion was greater for FID Sal treated cells (green and red dots), Bacterial lysis (green) decreased, and more FID Sal remained intact (red) after cancer cell infection as compared to ID Sal. D) While 60% of the control ID Sal lysed, only 40% of FID Sal lysed (**, P<0.01). E) 4T1 cells were infected with either control or flhDC expressing Salmonella. F) Control Salmonella were predominantly in vacuoles (red circle). However, a greater number of flhDC induced Salmonella resided in the cytosol (white circle). G) 90% of control Salmonella resided within vacuoles inside cancer cells as compared to 70% of flhDC induced bacteria (**, P<0.01). H) ID Sal were more likely to lyse intracellularly because the bacteria remained in vacuoles (White arrows). I) Although a significant fraction of FID Sal lysed inside cells (White arrows), a small but significant proportion of the bacteria evaded intracellular vacuoles and thus, did not lyse (Turquoise arrows). J) The presence of significant amounts of cytosolic, unlysed FID Sal was observed in vivo (white arrows).

FIGS. 11A-D. Overexpression of flhDC in Salmonella with impaired vacuole escape abilities maintains high cell invasion and rescues lysis efficiency. A) 4T1 cells infected with ID Sal had lower invasion but intracellularly lysed (green dots) with high efficiency. More FID Sal invaded 4T1 cancer cells but had lower lysis efficiency (green dots). The ΔsseJ FID Sal invaded 4T1 cancer cells and lysed intracellularly with high efficiency (red and green dots). B) ΔsseJ FID Sal invaded cancer cells three-fold more than ID Sal controls (**, P<0.01). C) ΔsseJ FID Sal lysed with 20% greater efficiency than ID Sal and (D) delivered 2.5 and 2 times more protein intracellularly than ID Sal or FID Sal, respectively (**, P<0.01).

FIG. 12. Modulating flhDC expression increases tumor selectivity and intracellular delivery distribution of engineered Salmonella. Salmonella lacking flhDC expression colonized tumors more selectively than strains without controlled flhDC expression. In tumors, flhDC expression enabled Salmonella to disperse and invade tumor cells. Expressing flhDC within an engineered, ΔsseJ strain enabled vacuolar retention of the Salmonella and lead to higher lysis efficiency and overall protein delivery within tumor cells.

FIGS. 13A-B. Genomic integration of inducible flhDC invades cancer cells as well as the parental and plasmid based inducible flhDC systems. A) After arabinose induction of both episomal and chromosomally integrated flhDC systems, Salmonella invaded (green dots) cancer cells (red) equally as well as the parental strain. B) The uninduced knock in strain was equally as noninvasive as the uninduced, plasmid-based system. After induction, EBV-002 with chromosomally integrated flhDC gene circuit was more invasive than either the uninduced plasmid based or genomically knocked in strain (*, P<0.05).

FIGS. 14A-B. Tuning flhD expression in EBV-002 with salicylic acid. A) EBV-002 was transformed with flhD constructs that were inducible with salicylic acid. The flhD gene was C-terminally tagged with either a low, medium or highly active degradation tag to suppress flhD activity in the uninduced state. As expected, none of the three strains invaded cancer cells without salicylic acid induction. However, after induction, only EBV-002 transformed with flhD containing low or moderate degradation tags invaded a large number of cells (green dots). EBV-002 containing flhD with a highly active degradation tag was only weakly invasive after induction. B) PBAD induction of flhD only increased intracellular invasion of EBV-002 two-fold compared to the uninduced control. EBV-002 invaded a significant number of cells without a degradation tag to suppress flhD activity in the uninduced state. However, salicylic induced samples (2) and (3) invaded cells approximately 30-fold more than the uninduced controls. EBV-002 containing a highly active degradation tag on flhD (sample 4) only invaded cancer cells five-fold more than the uninduced control. Induction of samples (2), (3) and (4) were all statistically significant at P<0.01.

FIGS. 15A-D. Clinical EBV-002 is triggered by aspirin to swim and invade cancer cells. EBV-002, which has a genomic deletion of flhD, was genetically engineered to express flhDC with a salicylic acid responsive genetic circuit. A) Without salicylic acid, the bacteria remained non-motile. After inducing the bacteria with salicylic acid, all bacteria were highly motile as shown by the paths of the bacteria (uninduced, blue; induced, red). B) Salicylic acid induced EBV-002 were 12.7 times more motile than the uninduced bacteria (***, P<0.001). C) Aspirin induction of flhDC robustly controlled cancer cell invasion of EBV-002. Aspirin Induced EBV-002 (green) invaded almost every cancer cell (white arrows). D) Aspirin induced EBV-002 invaded cancer cells 30-fold more than uninduced EBV-002 (***, P<0.001).

FIGS. 16A-B. Determination of the lowest amount of salicyclic acid needed to induce cell invasion of EBV-002. A) Concentrations above 500 nM salicylic acid induced microscopically visible amounts of intracellular EBV-002. B) A minimum of 500 nM of salicylic acid was sufficient to induce high levels of cell invasion (**, P<0.01).

FIGS. 17A-B. Biodistribution and protein delivery of EBV-003 and EBV-001. A) While EBV-003 colonization remained unchanged in the liver and spleen as compared to EBV-001, the EBV-003 strain colonized tumors 10.7-fold more than the first-generation strain (**, P<0.01). B) EBV-003 delivered 31 times more protein into tumors compared to EBV-001. Similar to EBV-001, EBV-003 did not deliver detectable quantities of protein into either the liver or spleen.

FIGS. 18A-C. Induction of flhD with salicylate increases penetration and intracellular invasion of EBV-003 within viable tumor tissue. A) Tumors containing uninduced (left) and induced (right) EBV-003. More bacteria (red Xs) were present intracellularly within or immediately adjacent to actively dividing tumor cells (solid red outline) in the induced as compared to the uninduced sample. B) Close histological examination revealed that uninduced EBV-003 residing near actively dividing tumor tissue did not penetrate into the tissue whereas, induction of flhD in EBV-003 significantly increased the presence of intracellular bacteria in actively dividing tumor cells (white arrows). C) There was a three-fold enrichment of EBV-003 invaded cancer cells in the flhD induced EBV-003 bacteria as compared to the uninduced sample (*, P<0.05).

FIGS. 19A-B. Intracellular protein delivery of EBV-003 within breast tumors. A) Induced EBV-003 delivered protein intracellularly into cells within actively dividing regions of tumors (white arrows). B) Intracellular protein delivery was only detected in one out of four mice with uninduced EBV-003. However, protein delivery was detected in five out of six mice with salicylate induced EBV-003.

FIGS. 20A-C. Colonization selectivity of EBV-003 in liver metastases of breast cancer versus healthy liver tissue. A) Aside from a small metastatic lesion the healthy liver tissue (left) contained a very limited number of EBV-003 colonies. On the other hand, a liver with several large metastatic lesions (right) was heavily colonized by EBV-003 (denoted with solid white boundaries). 85% of these colonies were within or immediately adjacent to actively dividing tumor cells indicated by the presence of dense blue nuclei (red arrows). B) On closer examination, the few colonies that were present in healthy liver tissue (1) were insignificantly small as compared to the bacteria within the metastatic lesions (2) indicating that on top of preferentially colonizing metastases, EBV-003 colonies grow orders of magnitude more within metastatic tissue as compared to healthy liver tissue (The red arrow pointing right indicates the portion of the liver with the metastatic lesion. The green arrow pointing left indicates the side of healthy liver tissue. The red line denotes the boundary between the two). C) The colony size of EBV-003 within metastatic lesions was 118-fold greater than the colony size within healthy liver tissue indicating the ability of the bacteria to grow orders of magnitude only within the tumor tissue (***, P=2.2×10⁻²⁶) FIGS. 21A-B. Intracellular Invasion of EBV-003 within spontaneous liver metastasis of EBV-003. A) A significant number of both flhDC uninduced and induced EBV-003 intracellularly invaded (white arrows) metastatic cancer cells within the liver. B) 87% and 83% of uninduced and induced EBV-003, respectively, intracellularly invaded or were immediately adjacent to cancer cells within metastatic lesions.

FIGS. 22A-B. Intracellular protein delivery of EBV-003 within metastatic breast cancer in the liver. A) EBV-003 (green) delivered protein (red) into metastatic breast cancer cells within the liver (white arrow). B) The flhDC induced EBV-003 delivered protein into metastatic tumor cells at a three-fold higher frequency as compared to uninduced EBV-003.

### DETAILED DESCRIPTION OF THE INVENTION

The majority of proteins are intracellular. Specifically targeting intracellular pathways specifically in cancer cells using macromolecular therapies increases the potential treatment options for any patient. However, macromolecular therapies that target intracellular pathways face significant barriers associated with tumor targeting, distribution, internalization and endosomal release. Engineered, non-pathogenic Salmonella selectively colonize tumors one thousand-fold more than any other organ, invade and deliver therapies cytosolically into cancer cells making the bacteria ideal delivery vehicles for cancer therapy.

However, a problem with using bacteria as an anti-cancer agent is their toxicity at the dose required for therapeutic efficacy and an obstacle in cancer gene therapy is the specific targeting of therapy directly to the cancer. Another issue to be addressed is systemic clearance of Salmonella. A further issue is the activity of cytosolic Salmonella (as compared to SCV Salmonella). A novel therapeutic platform for controlled colonization and/or invasion of engineered Salmonella in cancer cells and controlled gene and protein delivery in cancer cells, and therefore treatment for cancer, is provided herein.

To address these challenges, a bacterial delivery platform was developed that harnesses mechanisms unique to Salmonella to intracellularly deliver protein-based drugs. Salmonella sense the intracellular environment and accumulate inside cells when in tumors. Genetic circuits were engineered that force entry into cancer cells and release proteins from the endosome into the cytoplasm. Intracellular lysis makes the platform self-limiting and reduces the possibility of unwanted infection. Delivered nanobodies and protein interactors (NIPP1) bind to their targets and cause cell death. Delivery of caspase-3 to mice reduces growth of breast tumors and eliminates liver tumors. Intracellular delivery of protein-based drugs to tumors opens up the entire proteome for treatment.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, several embodiments with regards to methods and materials are described herein. As used herein, each of the following terms has the meaning associated with it in this section.

For the purposes of clarity and a concise description, features can be described herein as part of the same or separate embodiments; however, it will be appreciated that the scope of the invention may include embodiments having combinations of all or some of the features described.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

As used herein, the indefinite articles “a”, “an” and “the” should be understood to include plural reference unless the context clearly indicates otherwise.

The phrase “and/or,” as used herein, should be understood to mean “either or both” of the elements so conjoined, e.g., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, e.g., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

As used herein, the term “about” means plus or minus 10% of the indicated value. For example, about 100 means from 90 to 110. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about.”

The terms “individual,” “subject,” and “patient,” are used interchangeably herein and refer to any subject for whom diagnosis, treatment, or therapy is desired, including a mammal. Mammals include, but are not limited to, humans, farm animals, sport animals and pets. A “subject” is a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, orangutan) rat, sheep, goat, cow and bird.

The terms “treatment”, “treating” and the like are used herein to generally mean obtaining a desired pharmacologic and/or physiologic effect, such as arresting or inhibiting, or attempting to arrest or inhibit, the development or progression of a disorder and/or causing, or attempting to cause, the reduction, suppression, regression, or remission of a disorder and/or a symptom thereof. The effect may be prophylactic in terms of completely or partially preventing a disease or symptom thereof and/or may be therapeutic in terms of a partial or complete cure for a disease and/or adverse effect attributable to the disease. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms. Additionally, treatment can be applied to a subject or to a cell culture (in vivo or in vitro).

The terms “inhibit”, “inhibiting”, and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

“Expression” refers to the production of RNA from DNA and/or the production of protein directed by genetic material (e.g., RNA (mRNA)). Inducible expression, as opposed to constitutive expression (expressed all the time), is expression which only occurs under certain conditions, such as in the presence of specific molecule (e.g., arabinose) or an environmental que.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a nonnaturally-occurring nucleic acid is considered to be exogenous to a host once in the host. It is important to note that non-naturally occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally occurring nucleic acid. It follows that genomic DNA fragments produced by PCR or restriction endonuclease treatment as well as cDNAs are considered to be non-naturally occurring nucleic acid since they exist as separate molecules not found in nature. An exogenous sequence may therefore be integrated into the genome of the host. It also follows that any nucleic acid containing a promoter sequence and polypeptide-encoding sequence (e.g., cDNA or genomic DNA) in an arrangement not found in nature is non-naturally occurring nucleic acid. A nucleic acid that is naturally occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

Flagella are filamentous protein structures found in bacteria, archaea, and eukaryotes, though they are most commonly found in bacteria. They are typically used to propel a cell through liquid (i.e., bacteria and sperm). However, flagella have many other specialized functions. Flagella are usually found in gram-negative bacilli. Gram-positive rods (e.g., Listeria species) and cocci (some Enterococcus species, Vagococcus species) also have flagella.

Engineered Salmonella could be any strain of Salmonella designed to lyse and deliver protein intracellularly.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” is an amount sufficient to effect beneficial or desired result, such as a preclinical or clinical result. An effective amount can be administered in one or more administrations. The term “effective amount,” as applied to the compound(s), biologics and pharmaceutical compositions described herein, means the quantity necessary to render the desired therapeutic result. For example, an effective amount is a level effective to treat, cure, or alleviate the symptoms of a disorder and/or disease for which the therapeutic compound, biologic or composition is being administered. Amounts effective for the particular therapeutic goal sought will depend upon a variety of factors including the disorder being treated and its severity and/or stage of development/progression; the bioavailability, and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and like factors well known to one of skill in the relevant scientific art. Some variation in dosage can occur depending upon the condition of the subject being treated, and the physician or other individual administering treatment will, in any event, determine the appropriate dose for an individual patient.

As used herein, “disorder” refers to a disorder, disease or condition, or other departure from healthy or normal biological activity, and the terms can be used interchangeably. The terms would refer to any condition that impairs normal function. The condition may be caused by sporadic or heritable genetic abnormalities. The condition may also be caused by non-genetic abnormalities. The condition may also be caused by injuries to a subject from environmental factors, such as, but not limited to, cutting, crushing, burning, piercing, stretching, shearing, injecting, or otherwise modifying a subject's cell(s), tissue(s), organ(s), system(s), or the like.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double stranded DNA and cDNA. Furthermore, the terms, “nucleic acid,” “DNA,” “RNA” and similar terms also include nucleic acid analogs, i.e., analogs having other than a phosphodiester backbone. For example, the so called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; preferably in 7% (SDS), 0.5 M NaPO4, 1 mM EDTA at 50° C. with washing in 1×SSC, 0.1% SDS at 50° C.; preferably 7% SDS, 0.5 M NaPO4, 1 mM EDTA at 50° C. with washing in 0.5×SSC, 0.1% SDS at 50° C.; and more preferably in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984 Nucl. Acids Res. 12:387), and the BLASTN or FASTA programs (Altschul et al., 1990 Proc. Natl. Acad. Sci. USA. 1990 87:14:5509-13; Altschul et al., J. Mol. Biol. 1990 215:3:403-10; Altschul et al., 1997 Nucleic Acids Res. 25:3389-3402). The default settings provided with these programs are suitable for determining substantial similarity of nucleic acid sequences for purposes of the present invention.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, the term “pharmaceutically acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “regulate” refers to either stimulating or inhibiting a function or activity of interest.

By “small interfering RNAs (siRNAs)” is meant, inter alia, an isolated dsRNA molecule comprised of both a sense and an anti-sense strand. In one aspect, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

By the term “specifically binds to”, as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds preferentially to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

As used herein, the terms “including”, “includes”, “having”, “has”, “with”, or variants thereof, are intended to be inclusive similar to the term “comprising.”

The terms “comprises,” “comprising,” and the like can have the meaning ascribed to them in U.S. Patent Law and can mean “includes,” “including” and the like. As used herein, “including” or “includes” or the like means including, without limitation.

### I. Bacteria/Flagella

Bacteria useful in the invention include, but are not limited to, Clostridium, Bifidus, Escherichia coli or Salmonella, T3SS-dependent bacteria, such as shigella, salmonella and Yersinia Pestis. Further, E. coli can be used if the T3SS system is place in E. Coli.

### Salmonella

Examples of Salmonella strains which can be employed in the present invention include Salmonella typhi (ATCC No. 7251) and S. typhimurium (ATCC No. 13311). Attenuated Salmonella strains include S. typhi-aroC-aroD (Hone et al. Vacc. 9:810 (1991) S. typhimurium-aroA mutant (Mastroeni et al. Micro. Pathol. 13:477 (1992)) and Salmonella typhimurium 7207. Additional attenuated Salmonella strains that can be used in the invention include one or more other attenuating mutations such as (i) auxotrophic mutations, such as aro (Hoiseth et al. Nature, 291:238-239 (1981)), gua (McFarland et al Microbiol. Path., 3:129-141 (1987)), nad (Park et al. J. Bact, 170:3725-3730 (1988), thy (Nnalue et al. Infect. Immun., 55:955-962 (1987)), and asd (Curtiss, supra) mutations; (ii) mutations that inactivate global regulatory functions, such as cya (Curtiss et al. Infect. Immun., 55:3035-3043 (1987)), crp (Curtiss et al (1987), supra), phoP/phoQ (Groisman et al. Proc. Natl. Acad. Sci., USA, 86:7077-7081 (1989); and Miller et al. Proc. Natl. Acad. Sci., USA, 86:5054-5058 (1989)), phop.sup.c (Miller et al. J. Bact, 172:2485-2490 (1990)) or ompR (Dorman et al. Infect. Immun., 57:2136-2140 (1989)) mutations; (iii) mutations that modify the stress response, such as recA (Buchmeier et al. MoI. Micro., 7:933-936 (1993)), htrA (Johnson et al. MoI. Micro., 5:401-407 (1991)), htpR (Neidhardt et al. Biochem. Biophys. Res. Com., 100:894-900 (1981)), hsp (Neidhardt et al. Ann. Rev. Genet, 18:295-329 (1984)) and groEL (Buchmeier et al. Sci., 248:730-732 (1990)) mutations; mutations in specific virulence factors, such as IsyA (Libby et al. Proc. Natl. Acad. Sci., USA, 91:489-493 (1994)), pag or prg (Miller et al (1990), supra; and Miller et al (1989), supra), iscA or virG (d'Hauteville et al. MoI. Micro., 6:833-841 (1992)), plcA (Mengaud et al. Mol. Microbiol., 5:367-72 (1991); Camilli et al. J. Exp. Med, 173:751-754 (1991)), and act (Brundage et al. Proc. Natl. Acad. Sci., USA, 90:11890-11894 (1993)) mutations; (v) mutations that affect DNA topology, such as top A (Galan et al. Infect. Immun., 58: 1879-1885 (1990)); (vi) mutations that disrupt or modify the cell cycle, such as min (de Boer et al. Cell, 56:641-649 (1989)); (vii) introduction of a gene encoding a suicide system, such as sacB (Recorbet et al. App. Environ. Micro., 59:1361-1366 (1993); Quandt et al. Gene, 127:15-21 (1993)), nuc (Ahrenholtz et al. App. Environ. Micro., 60:3746-3751 (1994)), hok, gef, kil, or phlA (Molin et al. Ann. Rev. Microbiol., 47:139-166 (1993)); (viii) mutations that alter the biogenesis of lipopolysaccharide and/or lipid A, such as rFb (Raetz in Escherichia coli and Salmonella typhimurium, Neidhardt et al, Ed., ASM Press, Washington D.C. pp 1035-1063 (1996)), galE (Hone et al. J. Infect. Dis., 156:164-167 (1987)) and htrB (Raetz, supra), msbB (Reatz, supra; and U.S. Pat. No. 7,514,089); and (ix) introduction of a bacteriophage lysis system, such as lysogens encoded by P22 (Rennell et al. Virol, 143:280-289 (1985)), lamda murein transglycosylase (Bienkowska-Szewczyk et al. Mol. Gen. Genet., 184:111-114 (1981)) or S-gene (Reader et al. Virol, 43:623-628 (1971)).

The attenuating mutations can be either constitutively expressed or under the control of inducible promoters, such as the temperature sensitive heat shock family of promoters (Neidhardt et al. supra), or the anaerobically induced nirB promoter (Harbome et al. Mol. Micro., 6:2805-2813 (1992)) or repressible promoters, such as uapA (Gorfinkiel et al. J. Biol. Chem., 268:23376-23381 (1993)) or gcv (Stauffer et al. J. Bact, 176:6159-6164 (1994)).

In one embodiment, the bacterial delivery system is safe and based on a non-toxic, attenuated Salmonella strain that has a partial deletion of the msbB gene. This deletion diminishes the TNF immune response to bacterial lipopolysaccharides and prevents septic shock. In another embodiment, it also has a partial deletion of the purI gene. This deletion makes the bacteria dependent on external sources of purines and speeds clearance from non-cancerous tissues (13). In mice, the virulence (LD₅₀) of the therapeutic strain is 10,000-fold less than wild-type Salmonella (72, 73). In pre-clinical trials, attenuated Salmonella has been administered systemically into mice and dogs without toxic side effects (17, 27). Two FDA-approved phase I clinical trials have been performed and showed that this therapeutic strain can be safely administered to patients (20). In one embodiment, the strain of bacteria is VNP20009, a derivative strain of Salmonella typhimurium. Deletion of two of its genes—msbB and purI—resulted in its complete attenuation (by preventing toxic shock in animal hosts) and dependence on external sources of purine for survival. This dependence renders the organism incapable of replicating in normal tissue such as the liver or spleen, but still capable of growing in tumors where purine is available.

Further, insertion of a failsafe circuit into the bacterial vector prevents unwanted infection and defines the end of therapy without the need for antibiotics to remove the bacteria (e.g., salmonella).

### Flagella

1) flhDC Sequence

In one aspect, the flhDC sequence is the bicistronic, flhDC coding region found in the Salmonella Typhimurium 14028s strain or a derivative thereof

### Accession Number—

fhD-NCBI Reference Sequence: NC_016856.1
flhC-NCBI Reference Sequence: NC_016856.1

Bicistronic DNA sequence
(SEQ ID NO: 1)
ATGCATACATCCGAGTTGCTAAAACACATTTATGACATCA
ATTTGTCATATTTACTCCTTGCACAGCGTTTGATCGTCCA
GGACAAAGCATCTGCGATGTTCCGCCTCGGTATCAACGAA
GAGATGGCAAACACACTGGGCGCGTTGACCCTGCCGCAGA
TGGTCAAACTGGCGGAGACGAACCAGTTAGTTTGTCATTT
CCGGTTTGACGATCATCAGACGATCACCCGTTTGACTCAG
GATTCGCGCGTCGATGACTTACAGCAGATTCACACAGGTA
TCATGCTTTCAACGCGTCTGCTCAATGAAGTGGACGATAC
GGCGCGTAAGAAAAGGGCATGATAATGAGTGAAAAAAGCA
TTGTTCAGGAAGCTCGCGATATCCAGTTGGCGATGGAGTT
GATTAATCTTGGCGCTCGTCTACAAATGCTGGAAAGCGAA
ACACAGCTCAGCCGTGGTCGCCTCATCAGGCTGTACAAAG
AATTACGCGGTAGCCCGCCGCCTAAAGGGATGCTGCCATT
TTCGACAGACTGGTTTATGACCTGGGAGCAAAATATTCAT
GCCTCCATGTTCTGCAACGCCTGGCAATTTTTACTGAAGA
CCGGCTTATGCAGCGGTGTGGATGCGGTGATTAAAGCTTA
TCGGCTTTATCTTGAGCAGTGTCCGCAACCGCCTGAAGGG
CCGTTGTTGGCGCTGACTCGCGCATGGACGCTGGTGCGTT
TTGTTGAAAGTGGGTTGCTTGAATTGTCGAGCTGTAACTG
CTGCGGTGGGAACTTTATTACCCATGCGCATCAGCCCGTA
GGCAGCTTTGCGTGTAGTTTATGCCAGCCGCCATCCCGCG
CAGTAAAAAGACGTAAACTTTCCCGAGATGCTGCCGATAT
TATTCCACAACTGCTGGATGAACAGATCGAACAGGCTGTT
TAA
Protein sequence
flhD
(SEQ ID NO: 2)
MHTSELLKHIYDINLSYLLLAQRLIVQDKASAMFRLGINE
EMANTLGALTLPQMVKLAETNQLVCHFRFDDHQTITRLTQ
DSRVDDLQQIHTGIMLSTRLLNEVDDTARKKRA
flhC
(SEQ ID NO: 3)
MSEKSIVQEARDIQLAMELINLGARLQMLESETQLSRGRL
IRLYKELRGSPPPKGMLPFSTDWFMTWEQNIHASMFCNAW
QFLLKTGLCSGVDAVIKAYRLYLEQCPQPPEGPLLALTRA
WTLVRFVESGLLELSSCNCCGGNFITHAHQPVGSFACSLC
QPPSRAVKRRKLSRDAADIIPQLLDEQIEQAV

Other sequences can also be used to control flagella activity, these include, for example, motA, motB, flhE, cheZ, cheY cheB, cheR, cheM, cheW, cheA, fliA, fliY, fliZ, fluB, fliS, fluE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL.

motA, WP_000906312.1
>WP_000906312.1 MULTISPECIES: flagellar
motor stator protein MotA [Salmonella]
(SEQ ID NO: 4)
MLILLGYLVVIGTVFGGYVMTGGHLGALYQPAELVIIGGAGIGAF
IVGNNGKAIKGTMKAIPLLFRRSKYTKSMYMDLLALLYRLMAKSR
QQGMFSLERDIENPKESEIFASYPRILADAVMLDFIVDYLRLIIS
GNMNTFEIEALMDEEIETHESEAEVPANSLAMVGDSLPAFGIVAA
VMGVVHALASADRPAAELGALIAHAMVGTFLGILLAYGFISPLAT
VLRQKSAETTKMMQCVKITLLSNLNGYAPPIAVEFGRKTLYSSER
PSFIELEEHVRAVRNPNQQQTTEEA
motB, WP_000795653.1
>WP_000795653.1 MULTISPECIES: flagellar
motor protein MotB [Salmonella]
(SEQ ID NO: 5)
MKNQAHPIVVVKRRRHKPHGGGAHGSWKIAYADFMTAMMAFFLVM
WLISISSPKELIQIAEYFRTPLATAVTGGNRIANSESPIPGGGDD
YTQQQGEVEKQPNIDELKKRMEQSRLNKLRGDLDQLIESDPKLRA
LRPHLKIDLVQEGLRIQIIDSQNRPMFKTGSAEVEPYMRDILRAI
APVLNGIPNRISLAGHTDDFPYANGEKGYSNWELSADRANASRRE
LVAGGLDNGKVLRVVGMAATMRLSDRGPDDAINRRISLLVLNKQA
EQAILHENAESQNEBVSVLQQPAAAPPASVPTSPKAEPR
flhE, WP_001233619.1
>WP_001233619.1 MULTISPECIES: flagellar
protein FlhE [Salmonella]
(SEQ ID NO: 6)
MRKWLALLLFPLTVQAAGEGAWQDSGMGVTLNYRGVSASSSPLSA
RQPVSGVMTLVAWRYELNGPTPAGLRVRLCSQSRCVELDGQSGTT
HGFAHVPAVEPLRFVWEVPGGGRLIPALKVRSNQVIVNYR
cheZ, WP_000983586.1
>WP_000983586.1 MULTISPECIES: protein
phosphatase CheZ [Salmonella]
(SEQ ID NO: 7)
MMQPSIKPADEGSAGDIIARIGSLTRMLRDSLRELGLDQAIAEAA
EAIPDARDRLDYVVQMTAQAAERALNSVEASQPHQDAMEKEAKAL
TQRWDEWFDNPIELSDARELVTDTRQFLRDVPGHTSFTNAQLLDI
MMAQDFQDLTGQVIKRMMDVIQEIERQLLMVLLENIPEQSARPKR
ENESLLNGPQVDTSKAGVVASQDQVDDLLDSLGF
cheY WP_000763861.1
>WP_000763861.1 MULTISPECIES: chemotaxis
response regulator CheY [Salmonella]
(SEQ ID NO: 8)
MADKELKFLVVDDFSTMRRIVRNLLKELGFNNVEEAEDGVDALNK
LQAGGFGFIISDWNMPNMDGLELLKTIRADSAMSALPVLMVTAEA
KKENIIAAAQAGASGYVVKPFTAATLEEKLNKIFEKLGM
cheB, WP_000036392.1
>WP_000036392.1 MULTISPECIES: protein-glutamate
methylesterase/protein glutamine deamidase
[Salmonella]
(SEQ ID NO: 9)
MSKIRVLSVDDSALMRQIMTEIINSHSDMEMVATAPDPLVARDLI
KKFNPDVLTLDVEMPRMDGLDFLEKLMRLRPMPVVMVSSLTGKGS
EVTLRALELGAIDFVTKPQLGIREGMLAYSEMIAEKVRTAARARI
AAHKPMAAPTTLKAGPLLSSEKLIAIGASTGGTEAIRHVLQPLPL
SSPAVIITQHMPPGFTRSFAERLNKLCQISVKEAEDGERVLPGHA
YIAPGDKHMELARSGANYQIKIHDGPPVNRHRPSVDVLFHSVAKH
AGRNAVGVILTGMGNDGAAGMLAMYQAGAWTIAQNEASCVVFGMP
REAINMGGVSEVVDLSQVSQQMLAKISAGQAIRI
cheR, WP_000204362.1
>WP_000204362.1 MULTISPECIES: protein-
glutamate O-methyltransferase CheR
[Salmonella]
(SEQ ID NO: 10)
MTSSLPSGQTSVLLQMTQRLALSDAHFRRICQLIYQRAGIVLADH
KRDMVYNRLVRRLRALGLDDFGRYLSMLEANQNSAEWQAFINALT
TNLTAFFREAHHFPILAEHARRRHGEYRVWSAAASTGEEPYSIAI
TLADALGMAPGRWKVFASDIDTEVLEKARSGIYRLSELKTLSPQQ
LQRYFMRGTGPHEGLVRVRQELANYVEFSSVNLLEKQYNVPGPFD
AIFCRNVMIYFDKTTQEDILRRFVPLLKPDGLLFAGHSENFSNLV
REFSLRGQTVYALSKDKA
cheM, WP_000483274.1
>WP_000483274.1 MULTISPECIES: methyl-accepting
chemotaxis protein II [Salmonella]
(SEQ ID NO: 11)
MFNRIRVVTMLMMVLGVFALLQLVSGGLLFSSLQHNQQGFVISNE
LRQQQSELTSTWDLMLQTRINLSRSAARMMMDASNQQSSAKTDLL
QNAKTTLAQAAAHYANFKNMTPLPAMAEASANVDEKYQRYQAALA
ELIQFLDNGNMDAYFAQPTQGMQNALGEALGNYARVSENLYRQTF
DQSAHDYRFAQWQLGVLAVVLVLILMVVWFGIRHALLNPLARVIT
HIREIASGDLTKTLTVSGRNEIGELAGTVEHMQRSLIDTVTQVRE
GSDAIYSGTSEIAAGNTDLSSRTEQQASALEETAASMEQLTATVK
QNADNARQASQLAQSASETARHGGKVVDGVVNTMHEIADSSKKIA
DIISVIDGIAFQTNILALNAAVEAARAGEQGRGFAVVAGEVRNLA
SRSAQAAKEIKALIEDSVSRVDTGSVLVESAGETMTDIVNAVTRV
TDIMGEIASASDEQSRGIDQVALAVSEMDRVTQQNASLVQESAAA
AAALEEQASRLTQAVSAFRLASRPLAVNKPEMRLSVNAQSGNTPQ
SLAARDDANWETF
cheW, WP_000147295.1
>WP_000147295.1 MULTISPECIES: chemotaxis
protein CheW [Salmonella]
(SEQ ID NO: 12)
MTGMSNVSKLAGEPSGQEFLVFTLGNEEYGIDILKVQEIRGYDQV
TRIANTPAFIKGVTNLRGVIVPIVDLRVKFCEGDVEYDDNTVVIV
LNLGQRVVGIVVDGVSDVLSLTAEQIRPAPEFAVTLSTEYLTGLG
ALGERMLILVNIEKLLNSEEMALLDIAASHVA
cheA, WP_000061302.1
>WP_000061302.1 MULTISPECIES: chemotaxis
protein CheA [Salmonella]
(SEQ ID NO: 13)
MSMDISDFYQTFFDEADELLADMEQHLLDLVPESPDAEQLNAIFR
AAHSIKGGAGTFGFTILQETTHLMENLLDEARRGEMQLNTDIINL
FLETKDIMQEQLDAYKNSEEPDAASFEYICNALRQLALEAKGETT
PAVVETAALSAAIQEESVAETESPRDESKLRIVLSRLKANEVDLL
EEELGNLATLTDVVKGADSLSATLDGSVAEDDIVAVLCFVIEADQ
IAFEKVVAAPVEKAQEKTEVAPVAPPAVVAPAAKSAAHEHHAGRE
KPARERESTSIRVAVEKVDQLINLVGELVITQSMLAQRSNELDPV
NHGDLITSMGQLQRNARDLQESVMSIRMMPMEYVFSRFPRLVRDL
AGKLGKQVELTLVGSSTELDKSLIERIIDPLTHLVRNSLDHGIEM
PEKRLEAGKNVVGNLILSAEHQGGNICIEVTDDGAGLNRERILAK
AMSQGMAVNENMTDDEVGMLIFAPGFSTAEQVTDVSGRGVGMDVV
KRNIQEMGGHVEIQSKOGSGTTIRILLPLTLAILDGMSVRVAGEV
FILPLNAVMESLQPREEDLHPLAGGERVLEVRGEYLPLVELWKVF
DVDGAKTEATQGIVVILQSAGRRYALLVDQLIGQHQVVVKNLESN
YRKVPGISAATILGDGSVALIVDVSALQGLNREQRMAITAA
fliA, WP_001087453.1
>WP_001087453.1 MULTISPECIES: RNA polymerase
sigma factor FliA [Salmonella]
(SEQ ID NO: 14)
MNSLYTAEGVMDKHSLWQRYVPLVRHEALRLQVRLPASVELDDLL
QAGGIGLLNAVDRYDALQGTAFTTYAVQRIRGAMLDELRSRDWVP
RSVRRNAREVAQAMGQLEQELGRNATETEVAERLGIPVAEYRQML
LDTNNSQLFSYDEWREEHGDSIELVTEEHQQENPLHQLLEGDLRQ
RVMDAIESLPEREQLVLTLYYQEELNLKEIGAVLEVGESRVSQLH
SQAIKRLRTKLGKL
fliY, WP_000761635.1
>WP_000761635.1 MULTISPECIES: cystine ABC
transporter substrate-binding protein
[Salmonella]
(SEQ ID NO: 15)
MKLALLGRQALMGVMAVALVAGMSAKSFADEGLLNKVKERGTLLV
GLEGTYPPFSFQGEDGKLTGFEVDFAEALAKHLGVKASLKPTKWD
GMLASLDAKRIDVVINQVTISDVRKKKYDFSTPYTVSGIQALVKK
GNEGTIKTAADLQGKKVGVGLGTNYEEWLRQHVQGVDIRTYDDDP
TKYQDLRVGRIDAILVDRLAALDLVKKTKGTLAVTGDAFSRQESG
VALRKGNEDLLKAVDNAIAEMQKDGTLKALSEKWFGADVTQ
fliZ, WP_000218080.1
>WP_000218080.1 MULTISPECIES: flagella
biosynthesis regulatory protein FliZ
[Salmonella]
(SEQ ID NO: 16)
MTVQQPKRRPLSRYLKDFKHSQTHCAHCHKLLDRITLVRRGKIVN
KIAISQLDMLLDDAAWQREQKEWVALCRFCGDLHCKKQSDFFDII
GFKQYLFEQTEMSHGTVREYVVRLRRLGNYLSEQNISHDLLQDGF
LDESLAPWLPETSTNNYRIALRKYQQYKAHQQIAPROKSPFTASS
DIY
fliB, WP_000079794.1
>WP_000079794.1 MULTISPECIES: FliC/FljB
family flagellin [Salmonella]
(SEQ ID NO: 17)
MAQVINTNSLSLLTQNNLNKSQSALGTAIERLSSGLRINSAKDDA
AGQAIANRFTANIKGLTQASRNANDGISIAQTTEGALNEINNNLQ
RVRELAVQSANSTNSQSDLDSIQAEITQRLNEIDRVSGQTQFNGV
KVLAQDNTLTIQVGANDGETIDIDLKQINSQTLGLDSLNVQKAYD
VKDTAVTTKAYANNGTTLDVSGLDDAAIKAATGGTNGTASVTGGA
VKFDADNNKYFVTIGGFTGADAAKNGDYEVNVATDGTVTLAAGAT
KTTMPAGATTKTEVQELKDTPAVVSADAKNALIAGGVDATDANGA
ELVKMSYTDKNGKTIEGGYALKAGDKYYAADYDEATGAIKAKTTS
YTAADGTTKTAANQLGGVDGKTEVVTIDGKTYNASKAAGHDFKAQ
PELAEAAAKTTENPLQKIDAALAQVDALRSDLGAVQNRFNSAITN
LGNTVNNLSEARSRIEDSDYATEVSNMSRAQILQQAGTSVLAQAN
QVPQNVLSLLR
fliS, WP_000287764.1
>WP_000287764.1 MULTISPECIES: flagellar
export chaperone FliS [Salmonella]
(SEQ ID NO: 18)
MYTASGIKAYAQVSVESAVMSASPHQLIEMLFDGANSALVRARLF
LEQGDVVAKGEALSKAINIIDNGLKAGLDQEKGGEIATNLSELYD
YMIRRLLQANLRNDAQAIEEVEGLLSNIAEAWKQISPKASFQESR
fliE, WP_000719036.1
>WP_000719036.1 MULTISPECIES: flagellar
hook-basal body complex protein FliE [Salmonella]
(SEQ ID NO: 19)
MAAIQGIEGVISQLQATAMAARGQDTHSQSTVSFAGQLHAALDRI
SDRQAAARVQAEKFTLGEPGIALNDVMADMQKASVSMQMGIQVRN
KLVAAYQEVMSMQV
fliF, WP_001276834.1
>WP_001276834.1 MULTISPECIES: flagellar
M-ring protein FliF [Salmonella]
(SEQ ID NO: 20)
MSATASTATQPKPLEWLNRLRANPRIPLIVAGSAAVAIVVAMVLW
AKTPDYRTLFSNLSDQDGGAIVAQLTQMNIPYRFANGSGAIEVPA
DKVHELRLRLAQQGLPKGGAVGFELLDQEKFGISQFSEQVNYQRA
LEGELARTIETLGPVKSARVHLAMPKPSLFVREQKSPSASVTVTL
EPGRALDEGQISAVVHLVSSAVAGLPPGNVTLVDQSGHLLTQSNT
SGRDLNDAQLKFANDVESRIQRRIEAILSPIVGNGNVHAQVTAQL
DFANKEQTEEHYSPNGDASKATLRSROLNISEQVGAGYPGGVPGA
LSNQPAPPNEAPIATPPTNQQNAQNTPQTSTSTNSNSAGPRSTQR
NETSNYEVDRTIRHTKMNVGDIERLSVAVVVNYKTLADGKPLPLT
ADQMKQIEDLTREAMGFSDKRGDTLNVVNSPFSAVDNTGGELPFW
QQQSFIDQLLAAGRWLLVLVVAWILWRKAVRPQLTRRVEEAKAAQ
EQAQVRQETEEAVEVRLSKDEQLQQRRANQRLGAEVMSQRIREMS
DNDPRVVALVIRQWMSNDHE
fliJ, WP_000046981.1
>WP_000046981.1 MULTISPECIES: flagella
biosynthesis chaperone FliJ [Salmonella]
(SEQ ID NO: 21)
MAQHGALETLKDLAEKEVDDAARLLGEMRRGCQQAEEQLKMLID
YQNEYRSNLNTDMGNGIASNRWINYQQFIQTLEKAIEQHRLQLT
QWTQKVDLALKSWREKKQRLQAWQTLQDRQTAAALLAENRMDQK
KMDEFAQRAAMRKPE
fliL, WP_000132169.1
>WP_000132169.1 MULTISPECIES: flagellar basal
body-associated protein FliL [Salmonella]
(SEQ ID NO: 22)
MTDSAINKKSKRSIWIPLLVLITLAACATAGYSYWRMQQQPTTNA
KAEPAPPPAPVFFALDTFTVNLGDADRVLYIGVTLRLKDEATRAR
LNEYLPEVRSRLLLLFSRQNAAELSTEAGKQKLIAAIKETLAAPL
VAGQPKQVVTDVLYTAFILR
fliM, WP_000502811.1
>WP_000502811.1 MULTISPECIES: flagellar
motor switch protein FliM [Salmonella]
(SEQ ID NO: 23)
MGDSILSQAEIDALLNGDSDTKDEPTPGIASDSDIRPYDPNTQRR
VVRERLOALEIINERFARQFRMGLFNLLRRSPDITVGAIRIQPYH
EFARNLPVPTNLNLIHLKPLRGTGLVVFSPSLVFIAVDNLFGGDG
RFPTKVEGREFTHTEQRVINRMLKLALEGYSDAWKAINPLEVEYV
RSEMQVKFTNITTSPNDIVVNTPFHVEIGNLTGEFNICLPFSMIE
PLRELLVNPPLENSRHEDQNWRDNLVRQVQHSELELVANFADIPL
RLSQILKLKPGDVLPIEKPDRIIAHVDGVPVLTSQYGTVNGQYAL
RVEHLINPILNSLNEEQPK
fliN, WP_001282115.1
>WP_001282115.1 MULTISPECIES: flagellar
motor switch protein FliN [Salmonella]
(SEQ ID NO: 24)
MSDMNNPSDENTGALDDLWADALNEQKATTTKSAADAVFQQLGGG
DVSGAMQDIDLIMDIPVKLTVELGRTRMTIKELLRLTQGSVVALD
GLAGEPLDILINGYLIAQGEVVVVADKYGVRITDIITPSERMRRL
SR
fliO, WP_000978276.1
>WP_000978276.1 MULTISPECIES: flagellar type III
secretion system protein FliO [Salmonella]
(SEQ ID NO: 25)
MMKTEATVSQPTAPAGSPLMQVSGALIGIIALILAAAWVIKRMGF
APKGNSVRGLKVSASASLGPRERVVIVEVENARLVLGVTASQINL
LHTLPPAENDTEAPVAPPADFQNMMKSLLKRSGRS
fliP, WP_001253410.1
>WP_001253410.1 MULTISPECIES: flagellar type III
secretion system pore protein FliP [Salmonella]
(SEQ ID NO: 26)
MRRLLFLSLAGLWLFSPAAAAQLPGLISQPLAGGGQSWSLSVQTL
VFITSLTFLPAILLMMTSFTRIIIVFGLLRNALGTPSAPPNQVLL
GLALFLTFFIMSPVIDKIYVDAYQPFSEQKISMQEALDKGAQPLR
AFMLRQTREADLALFARLANSGPLQGPEAVPMRILLPAYVTSELK
TAFQIGFTIFIPFLIIDLVIASVLMALGMMMVPPATIALPFKLML
FVLVDGWQLLMGSLAQSFYS
fliQ, WP_000187355.1
>WP_000187355.1 MULTISPECIES: flagellar
biosynthesis protein FliQ [Salmonella]
(SEQ ID NO: 27)
MTPESVMMMGTEAMKVALALAAPLLLVALITGLIISILQAATQIN
EMTLSFIPKIVAVFIAIIVAGPWMLNLLLDYVRTLFSNLPYIIG
fliR, WP_000616953.1
>WP_000616953.1 MULTISPECIES: flagellar type
III secretion system protein FliR [Salmonella]
(SEQ ID NO: 28)
MIQVTSEQWLYWLHLYFWPLLRVLALISTAPILSERAIPKRVKLG
LGIMITLVIAPSLPANDTPLFSIAALWLAMQQILIGIALGFTMQF
AFAAVRTAGEFIGLQMGLSFATFVDPGSHLNMPVLARIMDMLAML
LFLTFNGHLWLISLLVDTFHTLPIGSNPVNSNAFMALARAGGLIF
LNGLMLALPVITLLLTLNLALGLLNRMAPQLSIFVIGFPLTLTVG
IMLMAALMPLIAPFCEHLFSEIFNLLADIVSEMPINNNP
fliG, WP_000067735.1
>WP_000067735.1 MULTISPECIES: flagellar
motor switch protein FliG [Salmonella]
(SEQ ID NO: 29)
MSNLSGTDKSVILLMTIGEDRAAEVFKHLSTREVQALSTAMANVR
QISNKQLTDVLSEFEQEAEQFAALNINANEYLRSVLVKALGEERA
SSLLEDILETRDTTSGIETLNFMEPQSAADLIRDEHPQIIATILV
HLKRSQAADILALFDERLRHDVMLRIATFGGVQPAALAELTEVLN
GLLDGQNLKRSKMGGVRTAAEIINLMKTQQEEAVITAVREFDGEL
AQKIIDEMFLFENLVDVDDRSIQRLLQEVDSESLLIALKGAEPPL
REKFLRNMSQRAADILRDDLANRGPVRLSQVENEQKAILLIVRRL
AETGEMVIGSGEDTYV
fliH, WP_000064163.1
>WP_000064163.1 MULTISPECIES: flagellar
assembly protein FliH [Salmonella]
(SEQ ID NO: 30)
MSNELPWQVWTPDDLAPPPETFVPVEADNVTLTEDTPEPELTAEQ
QLEQELAQLKIQAHEQGYNAGLAEGRQKGHAQGYQEGLAQGLEQG
QAQAQTQQAPIHARMQQLVSEFONTLDALDSVIASRLMQMALEAA
RQVIGQTPAVDNSALIKQIQQLLQQEPLFSGKPQLRVHPDDLQRV
EEMLGATLSLHGWRLRGDPTLHHGGCKVSADEGDLDASVATRWQE
LCRLAAPGVL
fliI, WP_000213257.1
>WP_000213257.1 MULTISPECIES: flagellum-
specific ATP synthase FliI [Salmonella]
(SEQ ID NO: 31)
MTTRLTRWLTALDNFEAKMALLPAVRRYGRLTRATGLVLEATGLO
LPLGATCIIERQDGPETKEVESEVVGFNGQRLFLMPLEEVEGILP
GARVYARNGHGDGLQSGKQLPLGPALLGRVLDGGGKPLDGLPAPD
TLETGALITPPFNPLQRTPIEHVLDTGVRAINALLTVGRGQRMGL
FAGSGVGKSVLLGMMARYTRADVIVVGLIGERGREVKDFIENILG
PDGRARSVVIAAPADVSPLLRMQGAAYATRIAEDFRDRGQHVLLI
MDSLTRYAMAQREIALAIGEPPATKGYPPSVFAKLPALVERAGNG
IHGGGSITAFYTVLTEGDDQQDPIADSARAILDGHIVLSRRLAEA
GHYPAIDIEASISRAMTALITEQHYARVRLFKQLLSSFQRNRDLV
SVGAYAKGSDPMLDKAITLWPQLEAFLQQGIFERADWEDSLQALD
LIFPTV
fliT, WP_000204899.1
>WP_000204899.1 MULTISPECIES: flagella
biosynthesis regulatory protein FliT [Salmonella]
(SEQ ID NO: 32)
MTSTVEFINRWQRIALLSQSLLELAQRGEWDLLLQQEVSYLQSIE
TVMEKQTPPGITRSIQDMVAGYIKQTLDNEQLLKGLLQQRLDELS
SLIGQSTRQKSLNNAYGRLSGMLLVPDAPGAS
fliD, WP_000146802.1
>WP_000146802.1 MULTISPECIES: flagellar
filament capping protein FliD [Salmonella]
(SEQ ID NO: 33)
MASISSLGVGSNLPLDQLLTDLTKNEKGRLTPITKQQSANSAKLT
AYGTLKSALEKFQTANTALNKADLFKSTVASSTTEDLKVSTTAGA
AAGTYKINVTQLAAAQSLATKTTFATTKEQLGDTSVTSRTIKIEQ
PGRKEPLEIKLDKGDTSMEAIRDAINDADSGIAASIVKVKENEFQ
LVLTANSGTDNTMKITVEGDTKLNDLLAYDSTTNTGNMQELVKAE
NAKLNVNGIDIERQSNTVTDAPQGITLTLTKKVTDATVTVTKDDT
KAKEAIKSWVDAYNSLVDTFSSLTKYTAVEPGEEASDKNGALLGD
SVVRTIQTGIRAQFANSGSNSAFKTMAEIGITQDGTSGKLKIDDD
KLTKVLKDNTAAARELLVGDGKETGITTKIATEVKSYLADDGIID
NAQDNVNATLKSLTKQYLSVSNSIDETVARYKAQFTQLDTMMSKL
NNTSSYLTQQFTAMNKS
fliC, WP_000079805.1
>WP_000079805.1 MULTISPECIES: FliC/FljB
family flagellin [Salmonella]
(SEQ ID NO: 34)
MAQVINTNSLSLLTQNNLNKSQSALGTAIERLSSGLRINSAKDDA
AGQAIANRFTANIKGLTQASRNANDGISIAQTTEGALNEINNNLQ
RVRELAVQSANSTNSQSDLDSIQAEITQRLNEIDRVSGQTQFNGV
KVLAQDNTLTIQVGANDGETIDIDLKQINSQTLGLDTLNVQQKYK
VSDTAATVTGYADTTIALDNSTFKASATGLGGTDQKIDGDLKFDD
TTGKYYAKVTVTGGTGKDGYYEVSVDKTNGEVTLAGGATSPLTGG
LPATATEDVKNVQVANADLTEAKAALTAAGVTGTASVVKMSYTDN
NGKTIDGGLAVKVGDDYYSATQNKDGSISINTTKYTADDGTSKTA
LNKLGGADGKTEVVSIGGKTYAASKAEGHNFKAQPDLAEAAATTT
ENPLQKIDAALAQVDTLRSDLGAVQNRFNSAITNLGNTVNNLTSA
RSRIEDSDYATEVSNMSRAQILQQAGTSVLAQANQVPQNVLSLLR
fljB, WP_000079794.1
>WP_000079794.1 MULTISPECIES: FliC/FljB
family flagellin [Salmonella]
(SEQ ID NO: 35)
MAQVINTNSLSLLTQNNLNKSQSALGTAIERLSSGLRINSAKDDA
AGQAIANRFTANIKGLTQASRNANDGISIAQTTEGALNEINNNLQ
RVRELAVQSANSTNSQSDLDSIQAEITQRLNEIDRVSGQTQFNGV
KVLAQDNTLTIQVGANDGETIDIDLKQINSQTLGLDSLNVQKAYD
VKDTAVTTKAYANNGTTLDVSGLDDAAIKAATGGTNGTASVTGGA
VKFDADNNKYFVTIGGFTGADAAKNGDYEVNVATDGTVTLAAGAT
KTTMPAGATTKTEVQELKDTPAVVSADAKNALIAGGVDATDANGA
ELVKMSYTDKNGKTIEGGYALKAGDKYYAADYDEATGAIKAKTTS
YTAADGTTKTAANQLGGVDGKTEVVTIDGKTYNASKAAGHDFKAQ
PELAEAAAKTTENPLQKIDAALAQVDALRSDLGAVQNRFNSAITN
LGNTVNNLSEARSRIEDSDYATEVSNMSRAQILQQAGTSVLAQAN
QVPQNVLSLLR
flgN, WP_000197547.1
>WP_000197547.1 MULTISPECIES: flagella
biosynthesis chaperone FlgN [Salmonella]
(SEQ ID NO: 36)
MTRLSEILDQMTTVLNDLKTVMDAEQQQLSVGQINGSQLQRITEE
KSSLLATLDYLEQQRRLEQNAQRSANDDIAERWQAITEKTQHLRD
LNQHNGWLLEGQIERNQQALEVLKPHQEPTLYGADGQTSVSHRGG
KKISI
flgM, WP_000020893.1
>WP_000020893.1 MULTISPECIES: anti-sigma-28
factor FlgM [Salmonella]
(SEQ ID NO: 37)
MSIDRTSPLKPVSTVQTRETSDTPVQKTRQEKTSAATSASVTLSD
AQAKLMQPGVSDINMERVEALKTAIRNGELKMDTGKIADSLIREA
QSYLQSK
flgA, WP_001194082.1
>WP_001194082.1 MULTISPECIES: flagellar basal
body P-ring formation protein FlgA
[Salmonella]
(SEQ ID NO: 38)
MQTLKRGFAVAALLFSPLTMAQDINAQLTTWFSQRLAGFSDEVVV
TLRSSPNLLPSCEQPAFSMTGSAKLWGNVNVVARCANEKRYLQVN
VQATGNYVAVAAPIARGGKLTPANVTLKRGRLDQLPPRTVLDIRQ
IQDAVSLRDLAPGQPVQLTMIRQAWRVKAGQRVQVIANGEGFSVN
AEGQAMNNAAVAQNARVRMTSGQIVSGTVDSDGNILINL
flgB, WP_000887043.1
>WP_000887043.1 MULTISPECIES: flagellar
basal body rod protein FlgB [Salmonella]
(SEQ ID NO: 39)
MLDRLDAALRFQQEALNLRAQRQEILAANIANADTPGYQARDIDF
ASELKKVMVRGREETGGVALTLTSSHHIPAQAVSSPAVDLLYRVP
DQPSLDGNTVDMDRERTQFADNSLKYQMGLTVLGSQLKGMMNVLQ
GGN
flgC, WP_001196448.1
>WP_001196448.1 MULTISPECIES: flagellar
basal body rod protein FlgC [Salmonella]
(SEQ ID NO: 40)
MALLNIFDIAGSALAAQSKRLNVAASNLANADSVTGPDGQPYRAK
QVVFQVDAAPGQATGGVKVASVIESQAPEKLVYEPGNPLADANGY
VKMPNVDVVGEMVNTMSASRSYQANIEVLNTVKSMMLKTLTLGQ
flgD, WP_000020450.1
>WP_000020450.1 MULTISPECIES: flagellar
hook assembly protein FlgD [Salmonella]
(SEQ ID NO: 41)
MSIAVNMNDPTNTGVKTTTGSGSMTGSNAADLQSSFLTLLVAQLK
NQDPTNPLONNELTTQLAQISTVSGIEKLNTTLGAISGQIDNSQS
LQATTLIGHGVMVPGTTILAGKGAEEGAVTSTTPFGVELQQPADK
VTATITDKDGRVVRTLEIGELRAGVHTFTWDGKQTDGTTVPNGSY
NIAITASNGGTQLVAQPLQFALVQGVTKGSNGNLLDLGTYGTTTL
DEVRQII
flgE, WP_000010567.1
>WP_000010567.1 MULTISPECIES: flagellar
hook protein FlgE [Salmonella]
(SEQ ID NO: 42)
MSFSQAVSGLNAAATNLDVIGNNIANSATYGFKSGTASFADMFAG
SKVGLGVKVAGITQDFTDGTTTNTGRGLDVAISQNGFFRLVDSNG
SVFYSRNGQFKLDENRNLVNMQGMQLTGYPATGTPPTIQQGANPA
PITIPNTLMAAKSTTTASMQINLNSTDPVPSKTPFSVSDADSYNK
KGTVTVYDSQGNAHDMNVYFVKTKDNEWAVYTHDSSDPAATAPTT
ASTTLKFNENGILESGGTVNITTGTINGATAATFSLSFLNSMQQN
TGANNIVATNQNGYKPGDLVSYQINNDGTVVGNYSNEQEQVLGQI
VLANFANNEGLASQGDNVWAATQASGVALLGTAGSGNFGKLTNGA
LEASNVDLSKELVNMIVAQRNYQSNAQTIKTQDQILNTLVNLR
flgF, WP_000349278.1
>WP_000349278.1 MULTISPECIES: flagellar
basal body rod protein FlgF [Salmonella]
(SEQ ID NO: 43)
MDHAIYTAMGAASQTLNQQAVTASNLANASTPGFRAQLNALRAVP
VDGLSLATRTLVTASTPGADMTPGQLDYTSRPLDVALQQDGWLVV
QAADGAEGYTRNGNIQVGPTGQLTIQGHPVIGEGGPITVPEGSEI
TIAADGTISALNPGDPPNTVAPVGRLKLVKAEGNEVQRSDDGLFR
LTAEAQAERGAVLAADPSIRIMSGVLEGSNVKPVEAMTDMIANAR
RFEMQMKVITSVDENEGRANQLLSMS
flgG, WP_000625851.1
>WP_000625851.1 MULTISPECIES: flagellar
basal-body rod protein FlgG [Salmonella]
(SEQ ID NO: 44)
MISSLWIAKTGLDAQQTNMDVIANNLANVSTNGFKRQRAVFEDLL
YQTIRQPGAQSSEQTTLPSGLQIGTGVRPVATERLHSQGNLSQTN
NSKDVAIKGQGFFQVMLPDGTSAYTRDGSFQVDQNGQLVTAGGFQ
VQPAITIPANALSITIGRDGVVSVTQQGQAAPVQVGQLNLTTFMN
DTGLESIGENLYIETQSSGAPNESTPGLNGAGLLYQGYVETSNVN
VAEELVNMIQVQRAYEINSKAVSTTDQMLOKLTQL
flgH, WP_001174897.1
>WP_001174897.1 MULTISPECIES: flagellar
basal body L-ring protein FlgH [Salmonella]
(SEQ ID NO: 45)
MQKYALHAYPVMALMVATLTGCAWIPAKPLVQGATTAQPIPGPVP
VANGSIFQSAQPINYGYQPLFEDRRPRNIGDTLTIVLQENVSASK
SSSANASRDGKTSFGFDTVPRYLQGLFGNSRADMEASGGNSFNGK
GGANASNTFSGTLTVTVDQVLANGNLHVVGEKQIAINQGTEFIRF
SGVVNPRTISGSNSVPSTQVADARIEYVGNGYINEAQNMGWLQRF
FLNLSPM
flgI, WP_001518955.1
>WP_001518955.1 MULTISPECIES: flagellar
basal body P-ring protein FlgI [Salmonella]
(SEQ ID NO: 46)
MFKALAGIVLALVATLAHAERIRDLTSVQGVRENSLIGYGLVVGL
DGTGDQTTQTPFTTQTLNNMLSQLGITVPTGTNMQLKNVAAVMVT
ASYPPFARQGQTIDVVVSSMGNAKSLRGGTLLMTPLKGVDSQVYA
LAQGNILVGGAGASAGGSSVQVNQLNGGRITNGAIIERELPTQFG
AGNTINLOLNDEDFTMAQQITDAINRARGYGSATALDARTVQVRV
PSGNSSQVRFLADIQNMEVNVTPQDAKVVINSRTGSVVMNREVTL
DSCAVAQGNLSVTVNRQLNVNQPNTPFGGGQTVVTPQTQIDLRQS
GGSLQSVRSSANLNSVVRALNALGATPMDLMSILQSMQSAGCLRA
KLEII
flgJ, WP_000578692.1
>WP_000578692.1 MULTISPECIES: flagellar
assembly peptidoglycan hydrolase FlgJ
[Salmonella]
(SEQ ID NO: 47)
MIGDGKLLASAAWDAQSLNELKAKAGQDPAANIRPVARQVEGMFV
QMMLKSMREALPKDGLFSSDQTRLYTSMYDQQIAQQMTAGKGLGL
ADMMVKQMTSGQTMPADDAPQVPLKFSLETVNSYQNQALTQLVRK
AIPKTPDSSDAPLSGDSKDFLARLSLPARLASEQSGVPHHLILAQ
AALESGWGQRQILRENGEPSYNVFGVKATASWKGPVTEITTTEYE
NGEAKKVKAKFRVYSSYLEALSDYVALLTRNPRYAAVTTAATAEQ
GAVALQNAGYATDPNYARKLTSMIQQLKAMSEKVSKTYSANLDNL
F
flgK, WP_000096425.1
>WP_000096425.1 MULTISPECIES: flagellar
hook-associated protein FlgK [Salmonella]
(SEQ ID NO: 48)
MSSLINHAMSGLNAAQAALNTVSNNINNYNVAGYTRQTTILAQAN
STLGAGGWIGNGVYVSGVQREYDAFITNQLRGAQNQSSGLTTRYE
QMSKIDNLLADKSSSLSGSLQSFFTSLQTLVSNAEDPAARQALIG
KAEGLVNQFKTTDQYLRDQDKQVNIAIGSSVAQINNYAKQIANLN
DQISRMTGVGAGASPNDLLDQRDQLVSELNKIVGVEVSVQDGGTY
NLTMANGYTLVQGSTARQLAAVPSSADPTRTTVAYVDEAAGNIEI
PEKLLNTGSLGGLLTFRSQDLDQTRNTLGQLALAFADAFNAQHTK
GYDADGNKGKDFFSIGSPVVYSNSNNADKTVSLTAKVVDSTKVQA
TDYKIVFDGTDWQVTRTADNTTFTATKDADGKLEIDGLKVTVGTG
AQKNDSFLLKPVSNAIVDMNVKVTNEAEIAMASESKLDPDVDTGD
SDNRNGQALLDLQNSNVVGGNKTFNDAYATLVSDVGNKTSTLKTS
STTQANVVKQLYKQQQSVSGVNLDEEYGNLQRYQQYYLANAQVLQ
TANALFDALLNIR
flgL WP_001223033.1
>WP 001223033.1 MULTISPECIES: flagellar
hook-associated protein FlgL [Salmonella]
(SEQ ID NO: 49)
MRISTQMMYEQNMSGITNSQAEWMKLGEQMSTGKRVTNPSDDPIA
ASQAVVLSQAQAQNSQYALARTFATQKVSLEESVLSQVTTAIQTA
QEKIVYAGNGTLSDDDRASLATDLQGIRDQLMNLANSTDGNGRYI
FAGYKTEAAPFDQATGGYHGGEKSVTQQVDSARTMVIGHTGAQIF
NSITSNAVPEPDGSDSEKNLFVMLDTAIAALKTPVEGNNVEKEKA
AAAIDKTNRGLKNSLNNVLTVRAELGTQLSELSTLDSLGSDRALG
QKLQMSNLVDVDWNSVISSYVMQQAALQASYKTFTDMQGMSLFQL
NR

### II. Vectors/Plasmids

In the present compositions and/or methods, DNA, RNA (e.g., a nucleic acid-based gene interfering agent) or protein may be produced by recombinant methods. The nucleic acid is inserted into a replicable vector for expression. Many such vectors are available. The vector components generally include, but are not limited to, one or more of the following: an origin of replication, one or more marker genes, an enhancer element, a promoter, and a transcription termination sequence and coding sequence. In some embodiments, for example in the utilization of bacterial delivery agents such as Salmonella, the gene and/or promoter (a sequence of interest) may be integrated into the host cell chromosome or may be presented on, for example, a plasmid/vector.

Expression vectors usually contain a selection gene, also termed a selectable marker. This gene encodes a protein necessary for the survival or growth of transformed host cells grown in a selective culture medium. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media.

Expression vectors can contain a promoter that is recognized by the host organism and is operably linked to the nucleic acid sequence, such as a nucleic acid sequence coding for an open reading frame. Promoters are untranslated sequences located upstream (5′) to the start codon of a structural gene (generally within about 100 to 1000 bp) that control the transcription of particular nucleic acid sequence to which they are operably linked. In bacterial cells, the region controlling overall regulation can be referred to as the operator. Promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Promoters suitable for use with prokaryotic hosts include the β-lactamase and lactose promoter systems, alkaline phosphatase, a tryptophan (trp) promoter system, hybrid promoters such as the tac promoter, and starvation promoters (Matin, A. (1994) Recombinant DNA Technology II, Annals of New York Academy of Sciences, 722:277-291). However, other known bacterial promoters are also suitable. Such nucleotide sequences have been published, thereby enabling a skilled worker to operably ligate them to a DNA coding sequence. Promoters for use in bacterial systems also can contain a Shine-Dalgarno (S.D.) sequence operably linked to the coding sequence.

Construction of suitable vectors containing one or more of the above-listed components employs standard ligation techniques. Isolated plasmids or DNA fragments are cleaved, tailored, and re-ligated in the form desired to generate the plasmids required.

In some embodiments of the invention, the expression vector is a plasmid or bacteriophage vector suitable for use in Salmonella, and the DNA, RNA and/or protein is provided to a subject through expression by an engineered Salmonella (in one aspect attenuated) administered to the patient. The term “plasmid” as used herein refers to any nucleic acid encoding an expressible gene and includes linear or circular nucleic acids and double or single stranded nucleic acids. The nucleic acid can be DNA or RNA and may comprise modified nucleotides or ribonucleotides and may be chemically modified by such means as methylation or the inclusion of protecting groups or cap- or tail structures.

One embodiment provides a Salmonella strain comprising a lysis gene or cassette operably linked to an intracellularly induced Salmonella promoter. In one embodiment, the promoter is a promoter for one of the genes in Salmonella pathogenicity island 2 type III secretion system (SPI2-T3SS) selected from the group SpiC/SsaB (accession no. CBW17423.1), SseF (accession no. CBW17434.1), SseG (accession no. CBW17435.1), SseI (accession no. CBW17087.1), SseJ (accession no. CBW17656.1 or NC_016856.1), SseK1 (accession no. CBW20184.1), SseK2 (accession no. CBW18209.1), SifA (accession no. CBW17257.1), SifB (accession no. CBW17627.1), PipB (accession no. CBW17123.1), PipB2 (accession no. CBW18862.1), SopD2 (accession no. CBW17005.1), GogB (accession no. CBW18646.2), SseL (accession no. CBW18358.1), SteC (accession no. CBW17723.1), SspH (accession no. STM4_1483), SspH2 (accession no. CBW18313.1), or SirP (examples/an embodiment of sequences that can be used in the instant compositions/methods are provided for by accession numbers and sequences provided throughout the specification; other sequences, including those with greater than about 80%, 85%, 90%, 95%, 96%, 97%, 98%, 99% and 100% identity may also be used in the composition/methods of the invention).

```
SpiC/SsaB (accession no. CBW17423.1):
(SEQ ID NO: 50)
    1 mseegfmlav lkgipliqdi raegnsrswi mtidghparg eifseafsis lflndleslp
  61 kpclayvtll laahpdvhdy aiqltadggw lngyyttsss seliaieiek hlaltcilkn
121 virnhhklys ggv
SseF (accession no. CBW17434.1):
(SEQ ID NO: 51)
    1 mkihipsaas nivdgnspps diqakevsip ppeipapgtp aapvlltpeq irqqrdyaih
  61 fmqytiralg atvvfglsva aavisggagl piailagaal viaigdacca yhnyqsicqq
121 keplqtasds valvvsalal kcgaslncan tlanclslli rsgiaismlv lplqfplpaa
181 eniaasldmg svitsvslta igavldycla rpsgddqens vdelhadpsv llaeqmaalc
241 qsattpalmd ssdhtsrgep
SseG (accession no. CBW17435.1):
(SEQ ID NO: 52)
    1 mkpvspnaqv ggqrpvnape esppcpslph petnmesgri gpqqgkervl aglakrviec
  61 fpkeifswqt vilggqilcc sagialtvls gggaplvala giglaiaiad vacliyhhkh
121 hlpmahdsig navfyiancf anqrksmaia kavslggrla ltatvmthsy wsgslglqph
181 llerlndity glmsftrfgm dgmamtgmqv ssplyrllaq vtpeqrape
SseI (accession no. CBW17087.1):
(SEQ ID NO: 53)
    1 mpfhigsgcl paiisnrriy riawsdtppe msswekmkef fcsthqaeal eciwtichpp
  61 agttredvvs rfellrtlay dgweenihsg lhgenyfcil dedsqeilsv tlddvgnytv
121 ncqgysethh ltmatepgve rtditynlts didaaaylee lkqnpiinnk imnpvgqces
181 lmtpvsnimn ekgfdniryr gifiwdkpte eiptnhfavv gnkegkdyvf dvsahqfenr
241 gmsnlngpli lsadewvcky rmatrrkliy ytdfsnssia anaydalpre lesesmagkv
301 fvtsprwint fkkqkyslig km
SseJ (accession no. CBW17656.1):
(SEQ ID NO: 54)
    1 mplsvgqgyf tssissekin aikesarlpe lslwekikay fftthhaeal ecifnlyhhq
  61 elnltpvqvr gayiklrala sqgckeqfii esqehadkli ikddngenil sievechpea
121 fglakeinks hpkpknislg ditrlvffgd slsdslgrmf ekthhilpsy gqyfggrftn
181 gftwteflss phflgkemln faeggstsas yscincigdf vsntdrqvas ytpshqdlai
241 fllgandymt lhkdnvimvv eqqiddieki isggvnnvlv mgipdlsltp ygkhsdekrk
301 lkdesiahna llktnveelk ekypqhkicy yetadafkvi meaasnigyd tenpythhgy
361 vhvpgakdpq ldicpqyvfn dlvhptqevh hcfaimlesf iahhyste
SseJ sequence (DNA)-Accession number-NCBI Reference Sequence: NC_016856.1
(SEQ ID NO: 55)
ATGCCATTGAGTGTTGGACAGGGTTATTTCACATCATCTATCAGTTCTGAAAAATTTAATGCGATAAAAGAAAGCGC
ACGCCTTCCGGAATTAAGTTTATGGGAGAAAATCAAAGCATATTTCTTTACCACCCACCATGCAGAGGCGCTCGAAT
GTATCTTTAATCTTTACCACCATCAGGAACTGAATCTAACACCGGTACAGGTTCGCGGAGCCTACATCAAACTTCGA
GCCTTAGCGTCTCAGGGATGTAAAGAACAGTTTATTATAGAATCACAGGAACACGCCGATAAGTTGATTATTAAAGA
TGATAATGGTGAAAATATTTTGTCTATTGAGGTTGAATGTCATCCGGAAGCTTTTGGTCTTGCAAAAGAAATCAATA
AATCACATCCCAAGCCCAAAAATATTTCTTTGGGTGATATTACCAGACTGGTATTTTTTGGCGACAGCTTGTCTGAC
TCCTTAGGGCGTATGTTTGAAAAAACACATCATATCTTACCCTCCTATGGTCAATACTTTGGCGGAAGGTTTACTAA
TGGATTTACCTGGACTGAGTTTTTATCATCTCCACACTTCTTAGGTAAAGAGATGCTTAATTTTGCTGAAGGGGGAA
GTACATCGGCAAGCTATTCCTGCTTTAATTGCATCGGTGACTTTGTATCAAATACGGACAGACAAGTCGCATCTTAC
ACCCCTTCTCACCAGGACCTGGCGATATTTTTATTGGGGGCTAATGACTATATGACACTACACAAAGATAATGTAAT
AATGGTCGTTGAGCAACAAATTGATGATATTGAAAAAATAATTTCCGGTGGAGTTAATAATGTTCTGGTCATGGGGA
TTCCCGATTTGTCTTTAACACCTTATGGCAAACATTCTGATGAAAAAAGAAAGCTTAAGGATGAAAGCATCGCTCAC
AATGCCCTGTTAAAAACTAATGTTGAAGAATTAAAAGAAAAATACCCCCAGCATAAAATATGCTATTACGAGACTGC
CGATGCATTTAAGGTGATAATGGAGGCGGCCAGTAATATTGGTTATGATACGGAAAACCCTTATACTCACCACGGCT
ATGTACATGTTCCCGGGGCTAAAGACCCTCAGCTAGATATATGTCCGCAATACGTCTTCAACGACCTTGTCCATCCA
ACCCAGGAAGTCCATCATTGTTTTGCCATAATGTTAGAAAGTTTTATAGCTCATCATTATTCCACTGAATAA
SseJ sequence (protein)
(SEQ ID NO: 56)
MPLSVGQGYFTSSISSEKFNAIKESARLPELSLWEKIKAYFFTTHHAEALECIFNLYHHQE
LNLTPVQVRGAYIKLRALASQGCKEQFIIESQEHADKLIIKDDNGENILSIEVECHPEAFG
LAKEINKSHPKPKNISLGDITRLVFFGDSLSDSLGRMFEKTHHILPSYGQYFGGRFTNGFT
WTEFLSSPHFLGKEMLNFAEGGSTSASYSCFNCIGDFVSNTDRQVASYTPSHQDLAIFLLG
ANDYMTLHKDNVIMVVEQQIDDIEKIISGGVNNVLVMGIPDLSLTPYGKHSDEKRKLKDES
IAHNALLKTNVEELKEKYPQHKICYYETADAFKVIMEAASNIGYDTENPYTHHGYVHVPGA
KDPQLDICPQYVFNDLVHPTQEVHHCFAIMLESFIAHHYSTE
SseK1 (accession no. CBW20184.1):
(SEQ ID NO: 57)
    1 mipplnryvp alsknelvkt vtnrdiqfts fngkdyplcf ldektpllfq wfernparfg
  61 kndipiinte knpylnniik aatiekerli gifvdgdffp gqkdafskle ydyenikviy
121 rndidfsmyd kklseiymen iskqesmpee krdchllqll kkelsdiqeg ndsliksyll
181 dkghgwfdfy rnmamlkagq lfleadkvgc ydlstnsgci yldadmiite klggiyipdg
241 iavhveridg rasmengiia vdrnnhpall agleimhtkf dadpysdgvc ngirkhfnys
301 lnedynsfcd fiefkhdnii mntsqftqss warhvq
SseK2 (accession no. CBW18209.1):
(SEQ ID NO: 58)
    1 marfnaaftr ikimfsrirg liscqsntqt iaptlsppss ghvsfagidy pllplnhqtp
  61 lvfqwfernp drfgqneipi intqknpyln niinaaiiek eriigifvdg dfskgqrkal
121 gkleqnyrni kviynsdlny smydkkltti ylenitklea qsaserdevl lngvkksled
181 vlknnpeetl isshnkdkgh lwfdfyrnlf llkgsdafle agkpgchhlq pgggciylda
241 dmlltdklgt lylpdgiaih vsrkdnhvsl engiiavnrs ehpalikgle imhskpygdp
301 yndwlskglr hyfdgshiqd ydafcdfief kheniimnts sltasswr
SifA (accession no. CBW17257.1):
(SEQ ID NO: 59)
    1 mpitigngfl kseiltnspr ntkeawwkvl wekikdfffs tgkakadrcl hemlfaerap
  61 trerlteiff elkelacasq rdrfqvhnph endatiilri mdqneenell ritqntdtfs
121 cevmgnlyfl mkdrpdilks hpqmtamikr ryseivdypl pstlclnpag apilsvpldn
181 iegylytelr kghldgwkaq ekatylaaki qsgiekttri lhhanisest qqnafletma
241 mcglkqleip pphthipiek mvkevlladk tfqaflvtdp stsqsmlaei veaisdqvfh
301 aifridpqai qkmaeeqltt lhvrseqqsg clccfl
SifB (accession no. CBW17627.1):
(SEQ ID NO: 60)
    1 mpitigrgfl ksemfsqsai sqrsfftllw ekikdffcdt qrstadqyik elcdvasppd
  61 aqrlidlfck lyelsspser gnfhfqhykd aecqytnlci kdgediplci mirqdhyyye
121 imnrtvlcd tqsahlkrys dinikastyv ceplcclipe rlqlslsggi tfsvdlknie
181 etliamaekg nlcdwkeger kaaissrinl giaqagvtai ddaiknkiaa kvientnlkn
241 aafepnyaqs svtqivyscl fkneilmnml eessshgllc lnelteyvtl qvhnslfsed
301 lsslvettkn eahhqs
PipB (accession no. CBW17123.1):
(SEQ ID NO: 61)
    1 mpitnaspen ilrylhaagt gtkeamksat sprgilewfv nfftcggvrr snerwfrevi
  61 gklttsllyv nknaffdgnk ifledvngct iclscgaase ntdpmviiev nkngktvtdk
121 vdserfwnvc rmlklmskhn iqqpdslite dgflnlrgvn lahkdfqged lskidasnad
181 frettlsnvn lvganlccan lhavnimgsn mtkanlthad ltcanmsgvn ltaailfgsd
241 ltdtklngak ldkialtlak altgadltgs qhtptplpdy ndrtlfphpi f
PipB2 (accession no. CBW18862.1):
(SEQ ID NO: 62)
    1 mersldslag maksafgagt saamrqatsp ktileyiinf ftcggirrrn etqyqeliet
  61 maetlkstmp drgaplpeni ilddmdgorv efnlpgenne agqvivrvsk gdhsetreip
121 lasfekicra llfrcefslp qdsviltaqg gmnlkgavlt ganltsenlc dadlsganle
181 gavlfmadce ganfkganls gtslgdsnfk nacledsimc gatldhanlt ganlqhasll
241 gcsmiecncs ganmdhtnls gatliradms gatlqgatim aaimegavlt ranlrkasfi
301 stnldgadla eanlnntcfk dctltdlrte datmststqt lfnefyseni
SopD2 (accession no. CBW17005.1):
(SEQ ID NO: 63)
    1 mpvtlsfgnr hnyeinhsrl arlmspdkee alymgvwdrf kdcfrthkkq evlevlytli
  61 hgcerenqae lnvditgmek ihaftqlkey anpsqqdrfv mrfdmnqtqv lfeidgkvid
121 kcnlhrllnv sencifkvme edeeelflki cikygekisr ypellegfan klkdavnedd
181 dvkdevyklm rsgedrkmec vewngtltee eknklrclqm gsfnittqff kigywelege
241 vlfdmvhptl syllqaykps lssdlietnt mlfsdvlnkd yddyqnnkre idailrriyr
301 shnntlfise ksscrnmli
GogB (accession no. CBW18646.2):
(SEQ ID NO: 64)
    1 mqyaytsnea tsnlellnkw riespdieke ernsiydkii eanhtgslsi tahhvtsipv
  61 fpdnlselnl sscytlesip nlpdglkslt isgnqtikis yfpdslesls idmqayeeny
121 tfpalpyglk sftacygkfl pplpphlssl slqnfseilc aelpykldkl dlqncpflpl
181 mkmlpeelke lsielirtvp gtviddilpd klkklsinfc dniklpvklp vnlksinlss
241 rtpiaweipt cnlpahidis tdgyvklnpe fltrsditfs nkpagdvlsf qpgdvvyglc
301 kardrvntlv nslyyfskkd iiiqntltda vwdrknravf nkdekiaerl ndvqrgiffr
361 eflsqhkkyn itedkysdls neecwiktsk aglefqtrlr ersvifvidn lvdaisdian
421 ktgkhgnsit ahelrwvyrn rhddlvkqnv kfflngeais hedvfslvgw dkykpknrnr
SseL (accession no. CBW18358.1):
(SEQ ID NO: 65)
    1 msdealtllf savengdqnc idllenlalr nddlghrvek flfdlfsgkr tgssdidkki
  61 nqaclvlhqi annditkdnt ewkklhapsr llymagsatt dlskkigiah kimgdqfaqt
121 dqeqvgvenl wcgarmlssd elaaatqglv qespllsvny piglihpttk enilstqlle
181 kiaqsglshn evilvntgdh wllolfykla ekikclifnt yydlnentkq eiieaakiag
241 isesdevnfi emnlqnnvpn goglfcyhti qllsnagqnd pattlrefae nfltlsveeq
301 alfntqtrrq iyeyslq
SteC (accession no. CBW17723.1):
(SEQ ID NO: 66)
    1 mpftfqignh scqiserylr diidnkrehv fstcekfidf frniftrrsl isdyreiynl
  61 lcqkkehpdi kgpfspgpfs krdedctrwr pllgyiklid asrpetidky tvevlahqen
121 mlllqmfydg vlvtetecse rcvdflketm fnynngeitl aalgndnlpp seagsngiye
181 afeqrlidfl ttpatasgye sgaidqtdas qpaaieafin spefqknirm rdieknkigs
241 gsygtvyrlh ddfvvkipvn ergikvdvns pehrnchpdr vskylnmand dknfsrsaim
301 ningkdvtvl vskyiqgqef dvedednyrm aeallksrgv ymhdinilgn ilvkegvlff
361 vdgdqivlsq esrqqrsvsl atrqleeqik ahhmiklkra etegntedve yykslitdld
421 aligeeeqtp apgrrfklaa peegtlvakv lkdelkk
SspH1 (accession no. STM14_1483):
(SEQ ID NO: 67)
    1 mfnirntqps vsmqaiagaa apeaspeeiv wekiqvffpq enyeeaqqcl aelchpargm
  61 lpdhissqfa rlkaltfpaw eenigenrdg inqfcildag skeilsitld dagnytvncq
121 gyseahdfim dtepgeecte faegasgtsl rpattvsqka aeydavwskw erdapagesp
181 graavvqemr dolnngnpvl nvgasglttl pdrlpphitt lvipdnnlts lpelpeglre
241 levsgnlqlt slpslpqglq klwaynnwla slptlppglg dlavsnnqlt slpemppalr
301 elrvsgnnlt slpalpsglq klwaynnrlt slpemspglq eldvshnqlt rlpqsltgls
361 saarvyldgn plsvrtlqal rdiighsgir ihfdmagpsv prearalhla vadwltsare
421 geaaqadrwq afglednaaa fslvldrlre tenfkkdagf kaqisswltq laedaalrak
481 tfamateats tcedrvthal hqmnnvqlvh naekgeydnn lqglvstgre mfrlatleqi
541 arekagtlal vddvevylaf qnklkeslel tsvtsemrff dvsgvtvsdl qaaelqvkta
601 ensgfskwil qwgplhsvle rkvperfnal rekqisdyed tyrklydevl kssglvddtd
661 aertigvsam dsakkefldg lralvdevlg syltarwrln
SspH2 (accession no. CBW18313.1):
(SEQ ID NO: 68)
    1 mpfhigsgcl patisnrriy riawsdtppe msswekmkef fcsthqteal eciwtichpp
  61 agttredvin rfellrtlay agweesihsg qhgenyfcil dedsqeilsv tlddagnytv
121 ncqgysethr ltldtaqgee gtghaegasg tfrtsflpat tapqtpaeyd avwsawrraa
181 paeesrgraa vvqkmracln ngnavlnvge sglttlpdel pahittlvip dnnltslpal
241 ppelrtlevs gnqltslpvl ppgllelsif snplthlpal psglcklwif gnqltslpvl
301 ppglqelsvs dnglaslpal pselcklway nnqltslpml psglqelsvs dnqlaslptl
361 pselyklway nnrltslpal psglkelivs gnrltslpvl pselkelmvs gnrltslpml
421 psgllslsvy rnqltripes lihlssettv nlegnplser tlqalreits apgysgpiir
481 fdmagasapr etralhlaaa dwlvparege papadrwhmf gqednadafs lfldrlsete
541 nfikdagfka qisswlaqla edealrantf amateatssc edrvtfflhq mknvqlvhna
601 ekgqydndla alvatgremf rlgkleqiar ekvrtlalvd eievwlayqn klkkslglts
661 vtsemrffdv sgvtvtdlqd aelqvkaaek sefrewilqw gplhrvlerk apervnalre
721 kqisdyeety rmlsdtelrp sglvgntdae rtigarames akktfldglr plveemlgsy
781 lnvqwrrn
```

In one embodiment, the Salmonella gene under the regulation of an inducible promoter is selected from ftsW (accession no. CBW16230.1), ftsA (accession no. CBW16235.1), ftsZ (accession no. CBW16236.1), murE (accession no. CBW16226.1), mukF (accession no. CBW17025.1), imp (accession no. CBW16196.1), secF (accession no. CBW16503.1), eno (accession no. CBW19030.1), hemH (accession no. CBWJ6582.1), tmk (accession no. CBW17233.1), dxs (accession no. CBW16516.1), uppS (accession no. CBW16324.1), cdsA (accession no. CBW16325.1), accA (accession no. CBWJ6335.1), pssA (accession no. CBW18718.1), msbA (accession no. CBW17017.1), tsf (accession no. CBW16320.1), trmD (accession no. CBW18749.1), cca (accession no. CBW19276.1), inJB (accession no. CBW19355.1), rpoA (accession no. CBW19477.1), rpoB (accession no. CBW20180.1), rpoC (accession no. CBW20181.1), holA (accession no. CBW16734.1), dnaC (accession no. CBW20563.1), or eng (EngA accession no. CBW18582.1; EngB accession no. CBW20039.1).

```
ftsW (accession no. CBW16230.1):
(SEQ ID NO: 69)
      1 mmasrdkdad slimydrtll wltfglaaig fvmvtsasmp vgqrlandpf lfakrdalyi
    61 flafclamvt lrlpmtfwqk ysttmliasi imllivlvvg ssvngasrwi algplriqpa
  121 eftklslicy lanylvrkvd evrnnlrgfl kpmgvilvla villaqpdlg tvvvlfvttl
  181 amlflagakl wqfiaiigmg isavillila epyrirrvts fwnpwedpfg sgyqltqslm
  241 afgrgeiwgq glgnsvqkle ylpeahtdfi faiigeelgy igvvlallmv ffvaframsi
  301 grkaleidhr fsgflacsig iwfsfqalvn vgaaagmlpt kgltlplisy ggssllimst
  361 aimfllridy etrlekaqaf trgsr
ftsA (accession no. CBW16235.1):
(SEQ ID NO: 70)
      1 mikatdrklv vgleigtakv aalvgevlpd gmvniigvgs cpsrgmdkgg vndlesvvkc
    61 vqraidqael madcqissvy lalsgkhisc qneigmvpis eeevtqedve nvvhtaksvr
  121 vrdehrvlhv ipqeyaidyq egiknpvgls gvrmqakvhl itchndmakn ivkavercgl
  181 kvdqlifagl aasysvlted erelgvcvvd igggtmdiav ytggalrhtk vipyagnvvt
  241 sdiayafgtp psdaeaikvr hgcalgsivg kdesvevpsv ggrpprslqr qtlaeviepr
  301 ytellnlvne eilqlqeqlr qqgvkhhlaa givltggaaq ieglaacaqr vfhtqvriga
  361 plnitgltdy aqepyystav gllhygkesh lngeaevekr vtasvgswik rinswlrkef
ftsZ (accession no. CBW16236.1):
(SEQ ID NO: 71)
      1 mfepmeltnd avikvigvgg gggnavehmv reriegveff avntdaqalr ktavgqtiqi
    61 gsgitkglga ganpevgrna adedrealra alegadmvfi aagmgggtgt gaapvvaeva
  121 kdlgiltvav vtkpfnfegk krmafaeqgi telskhvdsl itipndklik vlgrgislld
  181 afgaandvlk gavqgiaeli trpglmnvdf advrtvmsem gyammgsgva sgedraeeaa
  241 emaissplle didlsgargv lvnitagfdl rldefetvgn tirafasdna tvvigtsldp
  301 dmndelrvtv vatgigmdkr peitlvtnkq vqqpvldryq qhgmapltqe qktvakvvnd
  361 ntpqaakepd yldipaflrk qad
murE (accession no. CBW16226.1):
(SEQ ID NO: 72)
      1 madrnlrdll apwvaglpar elremtldsr vaaagdlfva vvghqadgrr yipqaiaqgv
    61 aaiiaeakde asdgeiremh gvpvvylsql nerlsalagr fyhepsenmr lvavtgtngk
  121 ttttqllaqw sqllgetsav mgtvgngllg kviptenttg savdvqhvla slvaqgatfg
  181 amevsshglv qhrvaalkfa asvftnlsrd hldyhgdmah yeaakwmlys thhhgqaivn
  241 addevgrrwl aslpdavavs meghinpnch grwlkaeave yhdrgatirf asswgegeie
  301 srlmgafnvs nlllalatll algypltdll ktaarlqpvc grmevftapg kptvvvdyah
  361 tpdalekalq aarlhcagkl wcvfgcggdr dkgkrplmga iaeefadivv vtddnprtee
  421 praiindila gmldagqvrv megraeavtn aimqakdndv vliagkghed yqivgtqrld
  481 ysdrvtaarl lgvia
mukF (accession no. CBW17025.1):
(SEQ ID NO: 73)
      1 msefsqtvpe lvawarkndf sislpvdrls fllavatlng erldgemseg elvdafrhvs
    61 dafeqtseti gvrannaind mvrqrlinrf tseqaegnai yrltplgigi tdyyirqref
  121 stlrlsmqls ivagelkraa daaaeggdef hwhrnvyapl kysvaeifds idltqrimde
  181 qqqqvkddia qlinkdwraa isscelllse tsgtlrelqd tleaagdklq anllriqdat
  241 mthddlhfvd rlvfdlqskl driiswgqqs idlwigydrh vhkfirtaid mdknrvfaqr
  301 lrqsvqtyfd dpwaltyana drlldmrdee malrddevtg elppdleyee fneireqlaa
  361 iieeqlaiyk trqtpldlgl vvreylaqyp rarhfdvari vidqavrlgv aqadftglpa
  421 kwqpindyga kvqahvidky
imp (accession no. CBW16196.1):
(SEQ ID NO: 74)
      1 mkkriptlla tmiasalysh qglaadlasq cmlgvpsydr plvkgdtndl pvtinadnak
    61 gnypddavft gnvdimqgns rlqadevqlh qkqaegqpep vrtvdalgnv hyddnqvilk
  121 gpkgwanlnt kdtnvwegdy qmvgrqgrgk adlmkqrgen rytilengsf tsclpgsdtw
  181 svvgsevihd reeqvaeiwn arfkvgpvpi fyspylqlpv gdkrrsgfli pnakyttkny
  241 fefylpyywn iapnmdatit phymhrrgni mwenefrylt qagegvmeld ylpsdkvyed
  301 dhpkegdkhr wlfnwqhsgv mdqvwrfnvd ytkvsdssyf ndfdskygss tdgyatqkfs
  361 vgyavqnfda tvstkqfqvf ndqntssysa epqldvnyyh ndlgpfdtri ygqavhfvnt
  421 kdnmpeatrv hleptinlpl snrwgslnte aklmathyqq tnldsynsdp nnknkledsv
  481 nrvmpqfkvd gkliferdma mlapgytqtl eprvqylyvp yrdqsgiyny dssllqsdyn
  541 glfrdrtygg ldriasanqv ttgvttriyd daaverfnvs vgqiyyftes rtgddnikwe
  601 nddktgslvw agdtywrise rwglrsgvqy dtrldsvats sssleyrrdq drlvqlnyry
  661 aspeyiqatl psyystaegy knginqvgav aswpiadrws ivgayyfdtn sskpadqmlg
  721 lqynsccyai rvgyerklng wdndkqhaiy dnaigfniel rglssnyglg tqemlrsnil
  781 pyqssm
secF (accession no. CBW16503.1):
(SEQ ID NO: 75)
      1 mageytveql nhgrkvydfm rwdfwafgis gllliaaivi mgvrgfnwgl dftggtviei
    61 tlekpaemdv mrealqkagy eepqlqnfgs shdimvrmpp tegetggqvl gskvvtiine
  121 atnqnaavkr iefvgpsvga dlaqtgamal lvalisilvy vgfrfewrla agvvialahd
  181 viitlgilsl fhieidltiv aslmsvigys lndsivvsdr irenfrkirr gtpyeifnvs
  241 ltqtlhrtli tsgttlvvil mlylfggpvl egfsltmlig vsigtassiy vasalalklg
  301 mkrehmlqqk vekegadqps ilp
eno (accession no. CBW19030.1):
(SEQ ID NO: 76)
      1 mskivkvigr eiidsrgnpt veaevhlegg fvgmaaapsg astgsreale lrdgdksrfl
    61 gkgvtkavga vngpiagail gkdakdqagi dkimidldgt enksnfgana ilavslanak
  121 aaaaakgmpl yehiaelngt pgkysmpvpm mniinggeha dnnvdigefm iqpvgaktvk
  181 eairmgsevf hhlakvlkgk gmntavgdeg gyapnlgsna ealaviaeav kaagyelgkd
  241 itlamdcaas efykdgkyvl agegnkafts eefthfleel tkqypivsie dgldesdwdg
  301 fayqtkvlgd kiqlvgddlf vtntkilkeg iekgiansil ikfnqigslt etlaaikmak
  361 dagytavish rsgetedati adlavgtaag qiktgsmsrs drvakynqli rieealgeka
  421 pyngrkeikg qa
hemH (accession no. CBW16582.1):
(SEQ ID NO: 77)
      1 mrqtktgill anlgtpdapt peavkrylkq flsdrrvvdt prllwwpllr gvilplrspr
    61 vaklyqsiwm dggsplmvys reqqqalaar lpdtpvalgm sygspslesa vdellasdvd
  121 hivvlplypq yscstvgavw delgrilark rripgisfir dyaddgayid alaksaresf
  181 arhgepdvll lsyhgipqry adegddypqr crdttrelvs alglppekvm mtfqsrfgre
  241 pwltpytdet lkmlgekgtg hiqvmcpgfa adcletleei aeqnreifle aggkkyayip
  301 alnatpehid mmlkltapyr
tmk (accession no. CBW17233.1):
(SEQ ID NO: 78)
      1 mgsnyivieg legagkttar dvvvetleql girnmiftre pggtqlaekl rslvldirsv
    61 gdevitdkae vlmfyaarvq lvetvikpal aqgvwvigdr hdlstqayqg ggrgidqtml
  121 atlrdavlgd frpdltlyld vtpevglkra rargdldrie qesfdffnrt rarylelaaq
  181 dsrirtidat qpldavmrdi ratvtkwvqe qaa
dxs (accession no. CBW16516.1):
(SEQ ID NO: 79)
      1 msfdiakypt lalvdstqel rllpkeslpk lcdelrryll dsvsrssghf asglgtvelt
    61 valhyvyntp fdqliwdvgh qayphkiltg rrdkigtirq kgglhpfpwr geseydvlsv
  121 ghsstsisag igiavaaeke gkdrrtvcvi gdgaitagma feamnhagdi rpdmlvilnd
  181 nemsisenvg alnnhlaqll sgklysslre ggkkvfsgvp pikellkrte ehikgmvvpg
  241 tlfeelgfny igpvdghdvm glistlknmr dlkgpqflhi mtkkgrgyep aekdpitfha
  301 vpkfdpssgc lpkssgglpg yskifgdwlc etaakdsklm aitpamregs gmvefsrkfp
  361 dryfdvaiae qhavtfaagl aiggykpvva iystflqray dqvihdvaiq klpvmfaidr
  421 agivgadgqt hqgafdlsyl rcipdmvimt psdenecrqm lftgyhyndg ptavryprgn
  481 aqgvaltple klpigkglvk rhgeklailn fgtlmpeaak vaealnatlv dmrfvkpldd
  541 tlilemaaqh dalvtleena imggagsgvn evlmahrkpv pvlniglpdf fipqgtqeea
  601 raelgldaag ieakikawla
uppS (accession no. CBW16324.1):
(SEQ ID NO: 80)
      1 mlsatqpvse nlpahgorhv aiimdgngrw akkqgkiraf ghkagaksvr ravsfaanng
    61 idaltlyafs senwnrpage vsalmelfvw aldsevkslh rhnvrlriig disrfnsrlq
  121 erirksealt ahntgltlni aanyggrwdi vqgvrqlaeq vqagvlrpdq ideerlgqqi
  181 cmhelapvdl virtggehri snillwqiay aelyftdvlw pdfdeqdfeg alhafanrer
  241 rfggtepgdd ka
cdsA (accession no. CBW16325.1):
(SEQ ID NO: 81)
      1 mlkyrlisaf vlipaviaal flippvgfai itlvvomlaa wewgqlsgfa arsqrvwlav
    61 lcglllalml fllpeyhhni rqplvemslw aslgwwvval llvlfypgsa aiwrnsktlr
  121 lifglltivp ffwgmlalra whydenhysg aiwllyvmil vwgadsgaym fgklfgkhkl
  181 apkvspgktw qgfigglata aviswgygmw anlnvapvil licsvvaala svlgdltesm
  241 fkreagikds ghlipghggi ldridsltaa vpvfacllll vfrtl
accA (accession no. CBW16335.1):
(SEQ ID NO: 82)
      1 mslnfldfeq piaeleakid sltavsrqde kldinideev hrlreksvel trkifadlga
    61 wqvaqlarhp qrpytldyvr lafdefdela gdrayaddka ivggiarleg rpvmiighqk
  121 gretkekirr nfgmpapegy rkallmema erfnmpiitf idtpgaypgv gaeergqsea
  181 iarnlremsr lnvpvictvi geggsggala igvgdkvnml qystysvisp egcasilwks
  241 adkaplaaea mgiiaprlke lklidsiipe plggahrnpe amaaslkaql ledladldvl
  301 stddlknrry qrlmsygya
pssA (accession no. CBW18718.1):
(SEQ ID NO: 83)
      1 mlskfkrnkh qqhlaqlpki sqsvddvdff ytpatfretl lekiasatqr icivalyleq
    61 ddggkgilda lyaakrqrpe ldvrvlvdwh raqrgrigaa asntnadwyc rlagenpgid
  121 vpvygvpint realgvlhfk gfiiddsvly sgasindvyl hqhdkyrydr yqlirnrqma
  181 dimfdwvtqn lmngrgvnrl dntqrpkspe ikndirlyrq elrdasyhfq gdandeqlsv
  241 tplvglgkss linktifhlm pcaehkltic tpyfnlpavl vrniiqllrd gkkveiivgd
  301 ktandfyipe depfkiigal pylyeinlrr flsrlqyyvn tdqlvvrlwk dddntyhlkg
  361 mwvddkwmll tgnnlnpraw rldlenaili hdpkqelapq rekelelirt httivkhyrd
  421 lqsiadypik vrklirrlrr iridrlisri l
msbA (accession no. CBW17017.1):
(SEQ ID NO: 84)
      1 mhndkdlstw qtfrrlwpti apfkagliva gialilnaas dtfmlsllkp llddgfgktd
    61 rsvllwmplv viglmilrgi tsyissycis wvsgkvvmtm rrrlfghmmg mpvaffdkqs
  121 tgtllsrity dseqvassss galitvvreg asiiglfimm fyyswqlsii lvvlapivsi
  181 airvvskrfr sisknmqntm gqvttsaeqm lkghkevlif ggqevetkrf dkvsnkmrlq
  241 gmkmvsassi sdpiiqlias lalafvlyaa sfpsvmdslt agtitvviss mialmrplks
  301 ltnvnaqfqr gmaacqtlfa ildseqekde gkrvidratg dlefrnvtft ypgrevpalr
  361 ninlkipagk tvalvgrsgs gkstiaslit rfydideghi lmdghdlrey tlaslrnqva
  421 lvsqnvhlfn dtvanniaya rteeysreqi eeaarmayam dfinkmdngl dtiigengvl
  481 lsggqrqria iarallrdsp ilildeatsa ldteseraiq aaldelqknr tslviahrls
  541 tieqadeivv vedgiiverg thsellaqhg vyaqlhkmqf gq
tsf (accession no. CBW16320.1):
(SEQ ID NO: 85)
      1 maeitaslvk elrertgagm mdckkaltea ngdielaien mrksgaikaa kkagnvaadg
    61 viktkidgnv afilevncqt dfvakdagfq afadkvldaa vagkitdvev lkaqfeeerv
  121 alvakigeni nirrvasleg dvlgsyqhga rigvlvaakg adeelvkqla mhvaaskpef
  181 vkpedvsadv vekeyqvqld iamqsgkpke iaekmvegrm kkftgevslt gqpfvmepsk
  241 svgqllkehn advtgfirfe vgegiekvet dfaaevaams kqs
trmD (accession no. CBW18749.1):
(SEQ ID NO: 86)
      1 mfigivslfp emfraitdyg vtgravkkgl lniqswsprd fahdrhrtvd drpygggpgm
    61 lmmvqplrda ihaakaaage gakviylspq grkldqagvs elatnqklil vcgryegvde
  121 rviqteidee wsigdyvlsg gelpamtlid svarfipgvl gheasaieds fadglldcph
  181 ytrpevlegm evppvllsgn haeirrwrlk qslgrtwlrr pellenlalt eeqarllaef
  241 ktehaqqqhk hdgma
cca (accession no. CBW19276.1):
(SEQ ID NO: 87)
      1 mkiylvggav rdallglpvk dkdwvvvgat pqemldagyq qvgrdfpvfl hpqtheeyal
    61 arterksgsg ytgftcyaap dvtleadlqr rdltinalar ddagqiidpy hgrrdlearl
  121 lrhvspafge dplrvlrvar faaryahlsf riadetlalm remtaagele hltpervwke
  181 tenalttrnp qvyfqvlrdc galrvlfpei dalfgvpapa kwhpeidtgv htlmtlsmaa
  241 mlspqldvrf atlchdlgkg ltpknlwprh hghgpagvkl veqlcqrlrv pndlrdlakl
  301 vaeyhdliht fpilqpktiv klfdaidawr kpqrveqial tseadvrgrt gfeasdypqg
  361 rwlreawqva qavptkevve agfkgieire eltkrriaav anwkekropn pas
infB (accession no. CBW19355.1):
(SEQ ID NO: 88)
      1 mtdvtlkala aerqvsvdrl vqqfadagir ksaddsvsaq ekqtllahln reavsgpdkl
    61 tlqrktrstl nipgtggksk svqievrkkr tfvkrdpqea erlaaeeqaq reaeegarre
  121 aeeqakreaq qkaereaaeq akreaaekak reaaekdkvs nqqtddmtkt aqaekarren
  181 eaaelkrkae eearrkleee arrvaeearr maeenkwtat pEBVedtsdy hvttsqharq
  241 aedendreve ggrgrgrnak aarpakkgkh aeskadreea raavrggkgg krkgsslqqg
  301 fqkpaqavnr dvvigetitv gelankmavk gsqvikammk lgamatinqv idqetaqlva
  361 eemghkvilr reneleeavm sdrdtgaaae prapvvtimg hvdhgktsll dyirstkvas
  421 geaggitqhi gayhvetdng mitfldtpgh aaftsmrarg aqatdivvlv vaaddgvmpq
  481 tieaiqhaka agvpvvvavn kidkpeadpd rvknelsqyg ilpeewgges qfvhvsakag
  541 tgidelldai llqaevlelk avrkgmasga viesfldkgr gpvatvlvre gtlhkgdivl
  601 cgfeygrvra mrnelgqevl eagpsipvei lglsgvpaag devtvvrdek karevalyrq
  661 gkfrevklar qqksklenmf anmtegevhe vnivlkadvq gsveaisdsl lklstdevkv
  721 kiigsgvggi tetdatlaaa snailvgfnv radasarkvi esesldlryy sviynlidev
  781 kaamsgmlsp elkqqiigla evrdvfkspk fgaiagcmvt egtikrhnpi rvlrdnvviy
  841 egeleslrrf kddvnevrng mecgigvkny ndvrvgdmie vfeiieiqrt ia
rpoA (accession no. CBW19477.1):
(SEQ ID NO: 89)
      1 mqgsvteflk prlvdieqvs sthakvtlep lergfghtlg nalrrillss mpgcavteve
    61 idgvlheyst kegvqedile illnlkglav rvqgkdevil tlnksgigpv taadithdgd
  121 veivkpqhvi chltdenasi smrikvqrgr gyvpastrih seederpigr llvdacyspv
  181 eriaynveaa rveqrtdldk lviemetngt idpeeairra atilaeqlea fvdlrdvrqp
  241 evkeekpefd pillrpvddl eltvrsancl kaeaihyigd lvqrtevell ktpnlgkksl
  301 teikdvlasr glslgmrlen wppasiade
rpoB (accession no. CBW20180.1):
(SEQ ID NO: 90)
      1 mvysytekkr irkdfgkrpq vldvpyllsi qldsfqkfie qdpeggygle aafrsvfpiq
    61 sysgnselqy vsyrlgEBVf dvqecqirgv tysaplrvkl rlviyereap egtvkdikeq
  121 evymgeiplm tdngtfving tervivsqlh rspgvffdsd kgkthssgkv lynariipyr
  181 gswldfefdp kdnlfvridr rrklpatiil ralnytteqi ldlffekvvf eirdnklqme
  241 liperlrget asfdieangk vyvekgrrit arhirqlekd dikhievpve yiagkvvskd
  301 yvdestgeli caanmelsld llaklsqsgh krietlftnd ldhgpyiset vrvdptndrl
  361 salveiyrmm rpgepptrea aeslfenlff sedrydlsav grmkfnrsll rdeiegsgil
  421 skddiidvmk klidirngkg evddidhlgn rrirsvgema enqfrvglvr veravkerls
  481 lgdldtlmpq dminakpisa avkeffgssq lsqfmdqnnp lseithkrri salgpggltr
  541 eragfevrdv hpthygrvcp ietpegpnig linslsvyaq tneygfletp yrrvvdgvvt
  601 deihylsaie egnyviaqan snlddeghfv edlvtorskg esslfsrdqv dymdvstqqv
  661 vsvgaslipf lehddanral mganmqrqav ptlradkplv gtgmeravav dsgvtavakr
  721 ggtvqyvdas rivikvnede mypgeagidi ynltkytrsn qntcinqmpc vslgEBVerg
  781 dvladgpstd lgelalgqnm rvafmpwngy nfedsilvse rvvqedritt ihiqelacvs
  841 rdtklgpeei tadipnvgea alskldesgi vyigaevtgg dilvgkvtpk getqltpeek
  901 llraifgeka sdvkdsslrv pngvsgtvid vqvftrdgve kdkraleiee mqlkqakkdl
  961 seelqileag lfsriravlv ssgveaekld klprdrwlel gltdeekqnq leqlaegyde
1021 lkhefekkle akrrkitqgd dlapgvlkiv kvylavkrri qpgdkmagrh gnkgviskin
1081 piedmpyden gtpvdivlnp lgvpsrmnig qilethlgma akgigdkina mlkqqqevak
1141 lrefiqrayd lgadvrqkvd lstfsddevl rlaenlrkgm piatpvfdga keaeikellk
1201 lgdlptsgqi tlfdgrtgeq ferpvtvgym ymlklnhlvd dkmharstgs yslvtqqplg
1261 gkaqfggqrf gemevwalea ygaaytlqem ltvksddvng rtkmyknivd gnhqmepgmp
1321 esfnvllkei rslginiele de
rpoC (accession no. CBW20181.1):
(SEQ ID NO: 91)
      1 mkdllkflka qtkteefdai kialaspdmi rswsfgevkk petinyrtfk perdglfcar
    61 ifgpvkdyec lcgkykrlkh rgvicekcgv evtqtkvrre rmghielasp tahiwflksl
  121 psrigllldm plrdiervly fesyvviegg mtnlerqqil teeqyldale efgdefdakm
  181 gaeaiqallk smdleqecet lreelnetns etkrkkltkr iklleafvqs gnkpewmilt
  241 vlpvlppdlr plvpldggrf atsdlndlyr rvinrnnrlk rlldlaapdi ivrnekrmlq
  301 eavdalldng rrgraitgsn krplksladm ikgkqgrfrq nllgkrvdys grsvitvgpy
  361 lrlhqcglpk kmalelfkpf iygklelrgl attikaakkm vereeavvwd ildevirehp
  421 vlinraptlh rlgiqafEBV liegkaiqlh plvcaaynad fdgdqmavhv pltleaqlea
  481 ralmmstnni lspangepii vpsqdvvlgl yymtrdcvna kgegmvltgp keaeriyrag
  541 laslharvkv riteyekden gefvahtslk dttvgrailw mivpkglpfs ivnqalgkka
  601 iskmlntcyr ilglkptvif adqtmytgfa yaarsgasvg iddmvipekk heiiseaeae
  661 vaeiqeqfqs glvtageryn kvidiwaaan drvskammdn lqtetvinrd gqeeqqvsfn
  721 siymmadsga rgsaaqirql agmrglmakp dgsiietpit anfreglnvl qyfisthgar
  781 kgladtalkt ansgyltrrl vdvaqdlvvt eddcgthegi lmtpvieggd vkeplrdrvl
  841 grvtaedvlk pgtadilvpr ntllheqwcd lleansvdav kvrsvvscdt dfgvcahcyg
  901 rdlarghiin kgeaigviaa qsigepgtql tmrtfhigga asraaaessi qvknkgsikl
  961 snvksvvnss gklvitsrnt elklidefgr tkesykvpyg avmakgdgeq vaggetvanw
1021 dphtmpvite vsgfirftdm idgqtitrqt deltglsslv vldsaerttg gkdlrpalki
1081 vdaqgndvli pgtdmpaqyf lpgkaivqle dgvqissgdt laripqesgg tkditgglpr
1141 vadlfearrp kepailaeia givsfgketk gkrrlvitpv dgsdpyeemi pkwrqlnvfe
1201 gervergdvi sdgpeaphdi lrlrgvhavt ryivnevqdv yrlqgvkind khievivrqm
1261 lrkatiesag ssdflegeqv eysrvkianr eleangkvga tfsrdllgit kaslatesfi
1321 saasfqettr vlteaavagk rdelrglken vivgrlipag tgyayhqdrm rrraageqpa
1381 tpqvtaedas aslaellnag lggsdne
holA (accession no. CBW16734.1):
(SEQ ID NO: 92)
      1 mirlypeqlr aqlneglraa ylllgndpll lqesqdairl aaasqgfeeh haftldpstd
    61 wgslfslcqa mslfasrqtl vlqlpengpn aamneqlatl sellhddlll ivrgnkltka
  121 qenaawytal adrsvqvscq tpeqaqlprw vaarakaqnl qlddaanqll cycyegnlla
  181 lagalerlsl lwpdgkltlp rveqavndaa hftpfhwvda llmgkskral hilqqlrleg
  241 sEBVillrtl qrellllvnl krqsahtplr alfdkhrvwq nrrpmigdal qrlhpaqlrq
  301 avqlltrtei tlkqdygqsv wadleglsll lchkaladvf idg
dnaC (accession no. CBW20563.1):
(SEQ ID NO: 93)
      1 mknvgdlmqr lqkmmpahit pafktgeell awqkeqgeir aaalarenra mkmqrtfnrs
    61 girplhqncs fdnyrvecdg qmnalskarq yvdefdgnia sfvfsgkpgt gknhlaaaic
  121 nelllrgksv liitvadims amkdtfsnre tseeqlindl snvdllvide igvqtesrye
  181 kviinqivdr rssskrptgm ltnsnmeemt kmlgervmdr mrlgnslwvn ftwdsyrsrv
  241 tgkey
eng (EngA accession no. CBW18582.1):
(SEQ ID NO: 94)
      1 mvpvvalvgr pnvgkstlfn rltrtrdalv adfpgltrdr kygraevegr eficidtggi
    61 dgtedgvetr maeqsllaie eadvvlimvd araglmpade aiakhlrsre kptflvankt
  121 dgldpdqavv dfyslglgei ypiaashgrg vlsllehvll pwmddvapqe evdedaeywa
  181 qfeaeqngee apeddfdpqs lpiklaivgr pnvgkstltn rilgeervvv ydmpgttrds
  241 iyipmerder eyvlidtagv rkrgkitdav ekfsviktlq aiedanvvll vidaregisd
  301 qdlsllgfil nsgrslvivv nkwdglsqev keqvketldf rlgfidfarv hfisalhgsg
  361 vgnlfesvre aydsstrrvs tamltrimtm avedhqpplv rgrrvklkya haggynppiv
  421 vihgnqvkdl pdsykrylmn yfrkslevmg tpiriqfkeg enpyankrnt ltptqmrkrk
  481 rlmkhikksk
EngB (accession no. CBW20039.1):
(SEQ ID NO: 95)
      1 mmsapdirhl psdcgievaf agrsnagkss alntltnqks lartsktpgr tqlinlfevv
    61 dgkrlvdlpg ygyaevpeem krkwqralge ylekrqslqg lvvlmdirhp lkdldqqmiq
  121 wavesniqvl vlltkadkla sgarkaqlnm vreavlafng dvqveafssl kkqgvdklrq
  181 kldswfsela pveeiqdge
```

Other inducible promotors for use in the invention, including to inducibly control flagella, include, but are not limited to:

pbad sequences
Full PBAD sequence with araC repressor
(from Invitrogen pbad-his-myc A plasmid)
(SEQ ID NO: 96)
ttatgacaacttgacggctacatcattcactttttcttcacaacc
ggcacggaactcgctcgggctggccccggtgcattttttaaatac
ccgcgagaaatagagttgatcgtcaaaaccaacattgcgaccgac
ggtggcgataggcatccgggtggtgctcaaaagcagcttcgcctg
gctgatacgttggtcctcgcgccagcttaagacgctaatccctaa
ctgctggcggaaaagatgtgacagacgcgacggcgacaagcaaac
atgctgtgcgacgctggcgatatcaaaattgctgtctgccaggtg
atcgctgatgtactgacaagcctcgcgtacccgattatccatcgg
tggatggagcgactcgttaatcgcttccatgcgccgcagtaacaa
ttgctcaagcagatttatcgccagcagctccgaatagcgcccttc
cccttgcccggcgttaatgatttgcccaaacaggtcgctgaaatg
cggctggtgcgcttcatccgggcgaaagaaccccgtattggcaaa
tattgacggccagttaagccattcatgccagtaggcgcgcggacg
aaagtaaacccactggtgataccattcgcgagcctccggatgacg
accgtagtgatgaatctctcctggcgggaacagcaaaatatcacc
cggtcggcaaacaaattctcgtccctgatttttcaccaccccctg
accgcgaatggtgagattgagaatataacctttcattcccagcgg
tcggtcgataaaaaaatcgagataaccgttggcctcaatcggcgt
taaacccgccaccagatgggcattaaacgagtatcccggcagcag
gggatcattttgcgcttcagccatacttttcatactcccgccatt
cagagaagaaaccaattgtccatattgcatcagacattgccgtca
ctgcgtcttttactggctcttctcgctaaccaaaccggtaacccc
gcttattaaaagcattctgtaacaaagcgggaccaaagccatgac
aaaaacgcgtaacaaaagtgtctataatcacggcagaaaagtcca
cattgattatttgcacggcgtcacactttgctatgccatagcatt
tttatccataagattagcggatcctacctgacgctttttatcgca
actctctactgtttctccatacccgttttttgggctaacaggagg
aattaacc
PBAD promoter sequence
(SEQ ID NO: 97)
Aagaaaccaattgtccatattgcatcagacattgccgtcactgcg
tcttttactggctcttctcgctaaccaaaccggtaaccccgctta
ttaaaagcattctgtaacaaagcgggaccaaagccatgacaaaaa
cgcgtaacaaaagtgtctataatcacggcagaaaagtccacattg
attatttgcacggcgtcacactttgctatgccatagcatttttat
ccataagattagcggatcctacctgacgctttttatcgcaactct
ctactgtttctccatacccgttttttgggctaacaggaggaatta
acc
AraC repressor protein
(SEQ ID NO: 98)
Atggctgaagcgcaaaatgatcccctgctgccgggatactcgttt
aatgcccatctggtggcgggtttaacgccgattgaggccaacggt
tatctcgatttttttatcgaccgaccgctgggaatgaaaggttat
attctcaatctcaccattcgcggtcagggggtggtgaaaaatcag
ggacgagaatttgtttgccgaccgggtgatattttgctgttcccg
ccaggagagattcatcactacggtcgtcatccggaggctcgcgaa
tggtatcaccagtgggtttactttcgtccgcgcgcctactggcat
gaatggcttaactggccgtcaatatttgccaatacggggttcttt
cgcccggatgaagcgcaccagccgcatttcagcgacctgtttggg
caaatcattaacgccgggcaaggggaagggcgctattcggagctg
ctggcgataaatctgcttgagcaattgttactgcggcgcatggaa
gcgattaacgagtcgctccatccaccgatggataatcgggtacgc
gaggcttgtcagtacatcagcgatcacctggcagacagcaatttt
gatatcgccagcgtcgcacagcatgtttgcttgtcgccgtcgcgt
ctgtcacatcttttccgccagcagttagggattagcgtcttaagc
tggcgcgaggaccaacgtatcagccaggcgaagctgcttttgagc
accacccggatgcctatcgccaccgtcggtcgcaatgttggtttt
gacgatcaactctatttctcgcgggtatttaaaaaatgcaccggg
gccagcccgagcgagttccgtgccggttgtgaagaaaaagtgaat
gatgtagccgtcaagttgtcataa
AraC protein sequence
(SEQ ID NO: 99)
MAEAQNDPLLPGYSFNAHLVAGLTPIEANGYLDFFIDRPLGMKGY
ILNLTIRGQGVVKNQGREFVCRPGDILLFPPGEIHHYGRHPEARE
WYHQWVYFRPRAYWHEWLNWPSIFANTGFFRPDEAHQPHESDLFG
QIINAGQGEGRYSELLAINLLEQLLLRRMEAINESLHPPMDNRVR
EACQYISDHLADSNFDIASVAQHVCLSPSRLSHLFRQQLGISVLS
WREDQRISQAKLLLSTTRMPIATVGRNVGFDDQLYFSRVFKKCTG
ASPSEFRAGCEEKVNDVAVKLS

### III. Therapeutic DNA, RNA and Peptides

The present invention delivers therapeutic DNA, RNA and/or peptides to cancer cells.

Gene silencing through RNAi (RNA-interference) by use of short interfering RNA (siRNA) can be used for therapeutic gene silencing. Short hairpin RNA (shRNA) transcribed from small DNA plasmids within the target cell has also been shown to mediate stable gene silencing and achieve gene knockdown at levels comparable to those obtained by transfection with chemically synthesized siRNA.

RNAi agents are agents that modulate expression of an RNA by an RNA interference mechanism. The RNAi agents employed in one embodiment of the subject invention are small ribonucleic acid molecules (also referred to herein as interfering ribonucleic acids), i.e., oligoribonucleotides, that are present in duplex structures, e.g., two distinct oligoribonucleotides hybridized to each other (e.g., an siRNA) or a single ribooligonucleotide that assumes a small hairpin formation to produce a duplex structure (e.g, shRNA).

dsRNA can be prepared according to any of a number of methods that are available in the art, including in vitro and in vivo methods, as well as by synthetic chemistry approaches. Single-stranded RNA can also be produced using a combination of enzymatic and organic synthesis or by total organic synthesis. The use of synthetic chemical methods enables one to introduce desired modified nucleotides or nucleotide analogs into the dsRNA.

In certain embodiments, instead of the RNAi agent being an interfering ribonucleic acid, e.g., an siRNA or shRNA as described above, the RNAi agent may encode an interfering ribonucleic acid, e.g., an shRNA, as described above. In other words, the RNAi agent may be a transcriptional template of the interfering ribonucleic acid. In these embodiments, the transcriptional template is typically a DNA that encodes the interfering ribonucleic acid. The DNA may be present in a vector, where a variety of different vectors are known in the art, e.g., a plasmid vector, a viral vector, etc.

Alternative the active agent may be a ribozyme. The term “ribozyme” as used herein for the purposes of specification and claims is interchangeable with “catalytic RNA” and means an RNA molecule that is capable of catalyzing a chemical reaction.

Exemplary target genes include, but are not limited to, EZH2 (accession number for human EZH2 mRNA is NM_004456). NIPP1 (accession number for human NIPP1 mRNA is NM_002713) and PP1 (accession numbers for human PP1 mRNA are PP1α mRNA: NM_002708; PP1β mRNA: NM_206876; PP1γ mRNA: NM_002710). EZH2, NIPP1 and PP1, would disrupt cancer cell processes and eliminate and/or diminish cancer stems cells. This will stop tumors from spreading/growing and prevent metastasis formation.

In another embodiment, the epigenetic target is at least one (e.g., mRNA) of NIPP1 (accession No. NM_002713); EZH2 (accession No. NM_004456); PP1α (accession No. NM_002708); PP1β (accession No. NM_206876); PP1γ (accession No. NM_002710); Suz12 (accession No. NM_015355); EED (accession No. NM_003797); EZH1 (accession No. NM_001991); RbAp48 (accession No. NM_005610); Jarid2 (accession No. NM_004973); YY1 (accession No. NM_003403); CBX2 (accession No. NM_005189); CBX4 (accession No. NM_003655); CBX6 (accession No. NM_014292); CBX7 (accession No. NM_175709); PHC1 (accession No. NM_004426); PHC2 (accession No. NM_198040); PHC3 (accession No. NM_024947); BMI1 (accession No. NM_005180); PCGF2 (accession No. NM_007144); ZNF134 (accession No. NM_003435); RING1 (accession No. NM_002931); RNF2 (accession No. NM_0072120; PHF1 (accession No. NM_024165); MTF2 (accession No. NM_007358); PHF19 (accession No. NM_001286840); SETD1A (accession No. NM_005255723); SETD1B (accession No. NM_015048); CXXC1 (accession No. NM_001101654); ASH2L (accession No. NM_004674); DPY30 (accession No. NM_032574); RBBP5 (accession No. NM_005057); WDR5 (accession No. NM_017588); KMT2A (accession No. NM_001197104); KMT2D (accession No. XM_006719616); KMT2B (accession No. NM_014727); KMT2C (accession No. NM_170606); KAT8 (accession No. NM_032188); KDM6A (accession No. NM_001291415); NCOA6 (accession No. NM_014071); PAGR1 (accession No. NM_024516); PAXIP1 (accession No. NM_007349); ASH1L (accession No. NM_018489); SMARCA2 (accession No. NM_003070); SMARCA4 (accession No. NM_001128844); BPTF (accession No. NM_182641); or SMARCA1 (accession No. NM_001282874).

```
NIPP1 (accession No. NM_002713):
(SEQ ID NO: 100)
        1 aaatgggagg gggagacgca agatggcggc agccgcgaac tccggctcta gcctcccgct
      61 gttcgactgc ccaacctggt gagtggcggg gcggccaggg ctagagtggc ccggccggag
    121 ctagcctggg ctggaagggc ggctcttttt ttacttttct gctgcgagcc gaacggctca
    181 gaaaccccgg aatggttgag gaaaaactgt ttgctgcacc gggccgggcg acgtgttgaa
    241 gaaccgagag cctggagccc aggcccagga actgaagaaa cccggggttg ggggctcaaa
    301 ggcgctcact taggcagccc ctttgagcga ttagccagtc gccggagcgc ttcgaggcct
    361 tggcccgaac ttacgcccaa ctcttgactg agtgcctggt gctctcgtgg agcatcgcat
    421 ctggcccctt cctgtacgtc ccgagcgcgc tcgagccagc cccggcccca accctacctc
    481 caagccccgc atccctctgt ggttgctgca tccctcgtgc ggcacttgtc tgtctgccac
    541 agagaatacg aggggcaggt aagccccctc ccggtttaca tctggatgta gtcaaaggag
    601 acaaactaat tgagaaactg attattgatg agaagaagta ttacttattt gggagaaacc
    661 ctgatttgtg tgactttacc attgaccacc agtcttgctc tcgggtccat gctgcacttg
    721 tctaccacaa gcatctgaag agagttttcc tgatagatct caacagtaaa cctgacagag
    781 ttcaacactg cccacaacaa gcggatttct acccttacca ttgaggaggg aaatctggac
    841 attcaaagac caaagaggaa gaggaagaac tcacgggtga cattcagtga ggatgatgag
    901 atcatcaacc cagaggatgt ggatccctca gttggtcgat tcaggaacat ggtgcaaact
    961 gcagtggtcc cagtcaagaa gaagcgtgtg gagggccctg gctccctggg cctggaggaa
  1021 tcagggagca ggcgcatgca gaactttgcc ttcagcggag gactctacgg gggcctgccc
  1081 cccacacaca gtgaagcagg ctcccagcca catggcatcc atgggacagc actcatcggt
  1141 ggcttgccca tgccataccc aaaccttgcc cctgatgtgg acttgactcc tgttgtgccg
  1201 tcagcagtga acatgaaccc tgcaccaaac cctgcagtct ataaccctga agctgtaaat
  1261 gaacccaaga agaagaaata tgcaaaagag gcttggccag gcaagaagcc cacaccttcc
  1321 ttgctgattt gatatttttg gtcatggaga agggtgggat tgggtgggaa tggggtggaa
  1381 gggtgatggg gagctaatga actagggaga aaaactttcc atgtgtgcgg tatcgtcttt
  1441 cagaatgtct cctggcatcc taaccatgta atatgacaat tgggggtggg gttgaaatag
  1501 cccataaaga cctgtcttca caacacttgc attgtagaga aaggcttctt atatcctttt
  1561 caatagactg ccctggctct ttcctaggcc ttccactacc tcctttcttt ctcccacttt
  1621 ctaggatcat ttttatgtaa agtcacatat cccaggccct caggttgaat ccagagctgt
  1681 agaggttaca gtagcatcac cagccttggg ggtccagagc ctaatttata ttcactatcc
  1741 ttccaagtcc cgggtagcag aagggttgcc atagatctca gtttgatcaa aaagaaggct
  1801 tagaattctg cagttaagct gaggtttaaa ctaaaaaatg tttccttggg tcagtggttt
  1861 tgaggtccag tagctaggct tttctctttt gtccttcctg ttggaatgaa aacatttcga
  1921 ttttccttca tctgtgactg gtgccataga cacaggttta tagttttaac ttacagtatt
  1981 gtttgaaatt tacctgtttt tcttgtcaaa cctgagcact cctcctgctg aagtttctta
  2041 tttaattcca gagtactgtc ctctactcta aggcattact tttaagtgta ttatgaaggc
  2101 agttttcaaa ggatatgacc agttggggta attcaaatta aaaaggaaaa gatttgtttg
  2161 gaagtaactg gtgtctctaa gaggaatttt tagatgtcag tttggaggct ctttcccccc
  2221 tcaattgaga gctcttgtta ttcagagctc caagactaga cctggctaac aaacatagga
  2281 gacaaagtta ggaaacattg atacaagctt tgtacagaga tttgtacatt tgtgtaatag
  2341 gccttttcat gctttatgtg tagcttttta cctgtaacct ttattacatt gtaaattaaa
  2401 cgtaactttt gtcatttggg tgcaggctgt gaatttgtct ctcagtcact gattgccact
  2461 gccatctgga aatgtttgct aaaggcacag tcactgggct tgggaggcaa tgctccatcc
  2521 ccattatatt acaaataaag atgccctaaa tgagtgtg
EZH2 (accession No. NM_004456)
(SEQ ID NO: 101)
        1 ggcggcgctt gattgggctg ggggggccaa ataaaagcga tggcgattgg gctgccgcgt
      61 ttggcgctcg gtccggtcgc gtccgacacc cggtgggact cagaaggcag tggagccccg
    121 gcggcggcgg cggcggcgcg cgggggcgac gcgcgggaac aacgcgagtc ggcgcgcggg
    181 acgaagaata atcatgggcc agactgggaa gaaatctgag aagggaccag tttgttggcg
    241 gaagcgtgta aaatcagagt acatgcgact gagacagctc aagaggttca gacgagctga
    301 tgaagtaaag agtatgttta gttccaatcg tcagaaaatt ttggaaagaa cggaaatctt
    361 aaaccaagaa tggaaacagc gaaggataca gcctgtgcac atcctgactt ctgtgagctc
    421 attgcgcggg actagggagt gttcggtgac cagtgacttg gattttccaa cacaagtcat
    481 cccattaaag actctgaatg cagttgcttc agtacccata atgtattctt ggtctcccct
    541 acagcagaat tttatggtgg aagatgaaac tgttttacat aacattcctt atatgggaga
    601 tgaagtttta gatcaggatg gtactttcat tgaagaacta ataaaaaatt atgatgggaa
    661 agtacacggg gatagagaat gtgggtttat aaatgatgaa atttttgtgg agttggtgaa
    721 tgcccttggt caatataatg atgatgacga tgatgatgat ggagacgatc ctgaagaaag
    781 agaagaaaag cagaaagatc tggaggatca ccgagatgat aaagaaagcc gcccacctcg
    841 gaaatttcct tctgataaaa tttttgaagc catttcctca atgtttccag ataagggcac
    901 agcagaagaa ctaaaggaaa aatataaaga actcaccgaa cagcagctcc caggcgcact
    961 tcctcctgaa tgtaccccca acatagatgg accaaatgct aaatctgttc agagagagca
  1021 aagcttacac tcctttcata cgcttttctg taggcgatgt tttaaatatg actgcttcct
  1081 acatcgtaag tgcaattatt cttttcatgc aacacccaac acttataagc ggaagaacac
  1141 agaaacagct ctagacaaca aaccttgtgg accacagtgt taccagcatt tggagggagc
  1201 aaaggagttt gctgctgctc tcaccgctga gcggataaag accccaccaa aacgtccagg
  1261 aggccgcaga agaggacggc ttcccaataa cagtagcagg cccagcaccc ccaccattaa
  1321 tgtgctggaa tcaaaggata cagacagtga tagggaagca gggactgaaa cggggggaga
  1381 gaacaatgat aaagaagaag aagagaagaa agatgaaact tcgagctcct ctgaagcaaa
  1441 ttctcggtgt caaacaccaa taaagatgaa gccaaatatt gaacctcctg agaatgtgga
  1501 gtggagtggt gctgaagcct caatgtttag agtcctcatt ggcacttact atgacaattt
  1561 ctgtgccatt gctaggttaa ttgggaccaa aacatgtaga caggtgtatg agtttagagt
  1621 caaagaatct agcatcatag ctccagctcc cgctgaggat gtggatactc ctccaaggaa
  1681 aaagaagagg aaacaccggt tgtgggctgc acactgcaga aagatacagc tgaaaaagga
  1741 cggctcctct aaccatgttt acaactatca accctgtgat catccacggc agccttgtga
  1801 cagttcgtgc ccttgtgtga tagcacaaaa tttttgtgaa aagttttgtc aatgtagttc
  1861 agagtgtcaa aaccgctttc cgggatgccg ctgcaaagca cagtgcaaca ccaagcagtg
  1921 cccgtgctac ctggctgtcc gagagtgtga ccctgacctc tgtcttactt gtggagccgc
  1981 tgaccattgg gacagtaaaa atgtgtcctg caagaactgc agtattcagc ggggctccaa
  2041 aaagcatcta ttgctggcac catctgacgt ggcaggctgg gggattttta tcaaagatcc
  2101 tgtgcagaaa aatgaattca tctcagaata ctgtggagag attatttctc aagatgaagc
  2161 tgacagaaga gggaaagtgt atgataaata catgtgcagc tttctgttca acttgaacaa
  2221 tgattttgtg gtggatgcaa cccgcaaggg taacaaaatt cgttttgcaa atcattcggt
  2281 aaatccaaac tgctatgcaa aagttatgat ggttaacggt gatcacagga taggtatttt
  2341 tgccaagaga gccatccaga ctggcgaaga gctgtttttt gattacagat acagccaggc
  2401 tgatgccctg aagtatgtcg gcatcgaaag agaaatggaa atcccttgac atctgctacc
  2461 tcctcccccc tcctctgaaa cagctgcctt agcttcagga acctcgagta ctgtgggcaa
  2521 tttagaaaaa gaacatgcag tttgaaattc tgaatttgca aagtactgta agaataattt
  2581 atagtaatga gtttaaaaat caacttttta ttgccttctc accagctgca aagtgttttg
  2641 taccagtgaa tttttgcaat aatgcagtat ggtacatttt tcaactttga ataaagaata
  2701 cttgaacttg tccttgttga atc
PP1α (accession No. NM_002708)
(SEQ ID NO: 102)
        1 gcggggccgc gggccggggg cggactgggg cgggcggaag gagagccagg ccggaaggag
      61 gctgccggag ggcgggaggc aggagcgggc caggagctgc tgggctggag cggcggcgcc
    121 gccatgtccg acagcgagaa gctcaacctg gactcgatca tcgggcgcct gctggaagtg
    181 cagggctcgc ggcctggcaa gaatgtacag ctgacagaga acgagatccg cggtctgtgc
    241 ctgaaatccc gggagatttt tctgagccag cccattcttc tggagctgga ggcacccctc
    301 aagatctgcg gtgacataca cggccagtac tacgaccttc tgcgactatt tgagtatggc
    361 ggtttccctc ccgagagcaa ctacctcttt ctgggggact atgtggacag gggcaagcag
    421 tccttggaga ccatctgcct gctgctggcc tataagatca agtaccccga gaacttcttc
    481 ctgctccgtg ggaaccacga gtgtgccagc atcaaccgca tctatggttt ctacgatgag
    541 tgcaagagac gctacaacat caaactgtgg aaaaccttca ctgactgctt caactgcctg
    601 cccatcgcgg ccatagtgga cgaaaagatc ttctgctgcc acggaggcct gtccccggac
    661 ctgcagtcta tggagcagat tcggcggatc atgcggccca cagatgtgcc tgaccagggc
    721 ctgctgtgtg acctgctgtg gtctgaccct gacaaggacg tgcagggctg gggcgagaac
    781 gaccgtggcg tctcttttac ctttggagcc gaggtggtgg ccaagttcct ccacaagcac
    841 gacttggacc tcatctgccg agcacaccag gtggtagaag acggctacga gttctttgcc
    901 aagcggcagc tggtgacact tttctcagct cccaactact gtggcgagtt tgacaatgct
    961 ggcgccatga tgagtgtgga cgagaccctc atgtgctctt tccagatcct caagcccgcc
  1021 gacaagaaca aggggaagta cgggcagttc agtggcctga accctggagg ccgacccatc
  1081 accccacccc gcaattccgc caaagccaag aaatagcccc cgcacaccac cctgtgcccc
  1141 agatgatgga ttgattgtac agaaatcatg ctgccatgct gggggggggt caccccgacc
  1201 cctcaggccc acctgtcacg gggaacatgg agccttggtg tatttttctt ttcttttttt
  1261 aatgaatcaa tagcagcgtc cagtccccca gggctgcttc ctgcctgcac ctgcggtgac
  1321 tgtgagcagg atcctggggc cgaggctgca gctcagggca acggcaggcc aggtcgtggg
  1381 tctccagccg tgcttggcct cagggctggc agccggatcc tggggcaacc catctggtct
  1441 cttgaataaa ggtcaaagct ggattctcgc aaaaaaaaaa aaaaaaaa
PP1β (accession No. NM_206876)
(SEQ ID NO: 103)
        1 gctgcgtgac gcggcggcgc gcaagggacg tgcggagtga gtggcgctgc gggtggggcc
      61 gtcggcggcg ctggtgagag aacgccgagc cgtcgccgca gcctccgccg ccgagaagcc
    121 cttgttcccg ctgctgggaa ggagagtctg tgccgacaag atggcggacg gggagctgaa
    181 cgtggacagc ctcatcaccc ggctgctgga ggtacgagga tgtcgtccag gaaagattgt
    241 gcagatgact gaagcagaag ttcgaggctt atgtatcaag tctcgggaga tctttctcag
    301 ccagcctatt cttttggaat tggaagcacc gctgaaaatt tgtggagata ttcatggaca
    361 atatacagat ttactgagat tatttgaata tggaggtttc ccaccagaag ccaactatct
    421 tttcttagga gattatgtgg acagaggaaa gcagtctttg gaaaccattt gtttgctatt
    481 ggcttataaa atcaaatatc cagagaactt ctttctctta agaggaaacc atgagtgtgc
    541 tagcatcaat cgcatttatg gattctatga tgaatgcaaa cgaagattta atattaaatt
    601 gtggaagacc ttcactgatt gttttaactg tctgcctata gcagccattg tggatgagaa
    661 gatcttctgt tgtcatggag gattgtcacc agacctgcaa tctatggagc agattcggag
    721 aattatgaga cctactgatg tccctgatac aggtttgctc tgtgatttgc tatggtctga
    781 tccagataag gatgtgcaag gctggggaga aaatgatcgt ggtgtttcct ttacttttgg
    841 agctgatgta gtcagtaaat ttctgaatcg tcatgattta gatttgattt gtcgagctca
    901 tcaggtggtg gaagatggat atgaattttt tgctaaacga cagttggtaa ccttattttc
    961 agccccaaat tactgtggcg agtttgataa tgctggtgga atgatgagtg tggatgaaac
  1021 tttgatgtgt tcatttcaga tattgaaacc atctgaaaag aaagctaaat accagtatgg
  1081 tggactgaat tctggacgtc ctgtcactcc acctcgaaca gctaatccgc cgaagaaaag
  1141 gtgaagaaag gaattctgta aagaaaccat cagatttgtt aaggacatac ttcataatat
  1201 ataagtgtgc actgtaaaac catccagcca tttgacaccc tttatgatgt cacaccttta
  1261 acttaaggag acgggtaaag gatcttaaat ttttttctaa tagaaagatg tgctacactg
  1321 tattgtaata agtatactct gttatagtca acaaagttaa atccaaattc aaaattatcc
  1381 attaaagtta catcttcatg tatcacaatt tttaaagttg aaaagcatcc cagttaaact
  1441 agatgtgata gttaaaccag atgaaagcat gatgatccat ctgtgtaatg tggttttagt
  1501 gttgcttggt tgtttaatta ttttgagctt gttttgtttt tgtttgtttt cactagaata
  1561 atggcaaata cttctaattt ttttccctaa acatttttaa aagtgaaata tgggaagagc
  1621 tttacagaca ttcaccaact attattttcc cttgtttatc tacttagata tctgtttaat
  1681 cttactaaga aaactttcgc ctcattacat taaaaaggaa ttttagagat tgattgtttt
  1741 aaaaaaaaat acgcacattg tccaatccag tgattttaat catacagttt gactgggcaa
  1801 actttacagc tgatagtgaa tattttgctt tatacaggaa ttgacactga tttggatttg
  1861 tgcactctaa tttttaactt attgatgctc tattgtgcag tagcatttca tttaagataa
  1921 ggctcatata gtattaccca actagttggt aatgtgatta tgtggtacct tggctttagg
  1981 ttttcattcg cacggaacac cttttggcat gcttaacttc ctggtaacac cttcacctgc
  2041 attggttttc tttttctttt ttctttcttt tttttttttt tttttttttt gagttgttgt
  2101 ttgtttttag atccacagta catgagaatc cttttttgac aagccttgga aagctgacac
  2161 tgtctctttt tcctccctct atacgaagga tgtatttaaa tgaatgctgg tcagtgggac
  2221 attttgtcaa ctatgggtat tgggtgctta actgtctaat attgccatgt gaatgttgta
  2281 tacgattgta aggcttatgt cactaaagat ttttattctg attttttcat aatcaaaggt
  2341 catatgatac tgtatagaca agctttgtag tgaagtatag tagcaataat ttctgtacct
  2401 gatcaagttt attgcagcct ttcttttcct atttcttttt tttaagggtt agtattaaca
  2461 aatggcaatg agtagaaaag ttaacatgaa gattttagaa ggagagaact tacaggacac
  2521 agatttgtga ttctttgact gtgacactat tggatgtgat tctaaaagct tttattgagc
  2581 attgtcaaat ttgtaagctt catagggatg gacatcatat ctataatgcc cttctatatg
  2641 tgctaccata gatgtgacat ttttgacctt aatatcgtct ttgaaaatgt taaattgaga
  2701 aacctgttaa cttacatttt atgaattggc acattgtatt acttactgca agagatattt
  2761 cattttcagc acagtgcaaa agttctttaa aatgcatatg tctttttttc taattccgtt
  2821 ttgttttaaa gcacatttta aatgtagttt tctcatttag taaaagttgt ctaattgata
  2881 tgaagcctga ctgatttttt ttttccttac agtgagacat ttaagcacac attttattca
  2941 catagatact atgtccttga catattgaaa tgattctttt ctgaaagtat tcatgatctg
  3001 catatgatgt attaggttag gtcacaaagg ttttatctga ggtgatttaa ataacttcct
  3061 gattggagtg tgtaagctga gcgatttcta ataaaatttt agttgtacac ttttagtagt
  3121 catagtgaag caggtctaga aaataagcct ttggcaggga aaaagggcaa tgttgattaa
  3181 tctcagtatt aaaccacatt aatctgtatc ccattgtctg gcttttgtaa attcatccag
  3241 gtcaagacta agtatgttgg ttaataggaa tccttttttt tttttttaaa gactaaatgt
  3301 gaaaaaataa tcactactta agctaattaa tattggtcat taaatttaaa ggatggaaat
  3361 ttatcatgtt taaaaattat tcaagcactc ttaaaaccac ttaaacagcc tccagtcata
  3421 aaaatgtgtt ctttacaaat atttgcttgg caacacgact tgaaataaat aaaactttgt
  3481 ttcttaggag aaaatgattc tgtaattcca gtgtcactaa tttatattgt tctttcctct
  3541 gatttttttc aggttagtga tttttttgta tacaatttaa tccaaatgtt atgacattca
  3601 gaaatcatga aacacagtag atatctgtta taatgtggtg tatcacatgg attataaagc
  3661 aaagttatgg tcgatttcta ttcttgaaag aatcaactac agtgaatcct ttgcatttga
  3721 agccttaaca tgcattgctt taattttgcc cagggacaaa ttttaataat cagcaagact
  3781 ggtttgtgca aagcgttgag tcatcaggta tttagagcct agccagctac ccagtatcca
  3841 tgctgccata tcccttcatt gtaaaaagta cctaaacatt cgtgaaatga ttttttttag
  3901 ctgaaaaatg ctggcaagaa gaattttaaa gcttaaaata ggtggtaaat ttgaagtatg
  3961 agtgtgttca cgagaaacat aggcttttca aaaaaatttt tattcaaggc aaagcaagga
  4021 acatcttgag atatgtctca agaatataaa gatgtattat tttaagccaa ggagctgaaa
  4081 tatatctcag tttataaatt caggtatatt ctttttgtct ccatggcaac cataactttt
  4141 gaaccaaaaa aaattgtttt tacatcttta tgctgaaaat gtgtttagat taggaatatg
  4201 gtcgggctga atttgctgtt gctccctaac caaatccacc tcttgttttc cttgtgagtc
  4261 catggctaaa tcaaagctgc ccctgagaag agacttaatc caagcctgat tgtactagtg
  4321 gcatcactta gaagtaggct ttccctcttc ctagtagatc tcaatgtttt ataattcctt
  4381 aaaacagctg aaaattggga caacatactt tacgcaatga acagtagtta aataggaaat
  4441 aaactagttc catataagta tacacctaga gttttaatta cctttataat gtttcttaaa
  4501 agtgaaactt agatacaatt gtgattggat acttagatac taagtgaaac ttagtgtaac
  4561 aattttgatc tgttaaattg gattttacat gtacatttga atgccagaat ttctaaataa
  4621 atcccctggt taggaaattt taaaagtcaa agcttgtttt cttcaaccac taccttctac
  4681 attggttgac ttagaccgta agctttttaa gtttctcatt gtaatttacc ttctcatgca
  4741 gattgctgat gttttattaa accttatttt tacaaaaatg aaaaaa
PPly (accession No. NM_002710)
(SEQ ID NO: 104)
        1 taaagaagtc ccggccgggc cgctgcactc cccgcgcgca tccgtgcgcc gcccgaggct
      61 gtctaaggag tcggcggcca ttttgttctt ctcgtggttc cagtggggag agaaggagga
    121 agtagggagc ggggtggcag gggggggacc cgccgcggct gctgccaccg ccgccaccac
    181 cgcctctgct cgtggcgtgg gaaaggaggt gtgagtcccg ggcgcgagcc ggcggcggcg
    241 ccgctgcggg agggtcggcg gtgggaaggc gatggcggat ttagataaac tcaacatcga
    301 cagcattatc caacggctgc tggaagtgag agggtccaag cctggtaaga atgtccagct
    361 tcaggagaat gaaatcagag gactgtgctt aaagtctcgt gaaatctttc tcagtcagcc
    421 tatcctacta gaacttgaag caccactcaa aatatgtggt gacatccatg gacaatacta
    481 tgatttgctg cgactttttg agtacggtgg tttcccacca gaaagcaact acctgtttct
    541 tggggactat gtggacaggg gaaagcagtc attggagacg atctgcctct tactggccta
    601 caaaataaaa tatcctgaga atttttttct tctcagaggg aaccatgaat gtgccagcat
    661 caacagaatt tatggatttt atgatgaatg taaaagaaga tacaacatta aactatggaa
    721 aactttcaca gactgtttta actgtttacc gatagcagcc atcgtggatg agaagatatt
    781 ctgctgtcat ggaggtttat caccagatct tcaatctatg gagcagattc ggcgaattat
    841 gcgaccaact gatgtaccag atcaaggtct tctttgtgat cttttgtggt ctgaccccga
    901 taaagatgtc ttaggctggg gtgaaaatga cagaggagtg tccttcacat ttggtgcaga
    961 agtggttgca aaatttctcc ataagcatga tttggatctt atatgtagag cccatcaggt
  1021 ggttgaagat ggatatgaat tttttgcaaa gaggcagttg gtcactctgt tttctgcgcc
  1081 caattattgc ggagagtttg acaatgcagg tgccatgatg agtgtggatg aaacactaat
  1141 gtgttctttt cagattttaa agcctgcaga gaaaaagaag ccaaatgcca cgagacctgt
  1201 aacgcctcca aggggtatga tcacaaagca agcaaagaaa tagatgtcgt tttgacactg
  1261 cctagtcggg acttgtaaca tagagtatat aaccttcatt tttaagactg taatgtgtac
  1321 tggtcagctt gctcagatag atctgtgttt gtgggggccc ttccttccat ttttgattta
  1381 gtgaatggca tttgctggtt ataacagcaa atgaaagact cttcactcca aaaagaaaag
  1441 tgttttgttt tttaattctc tgttcctttt gcaaacaatt ttaatgatgg tgttaaagct
  1501 gtacacccca ggacagttta tcctgtctga ggagtaagtg tacaattgat cttttttaat
  1561 tcagtacaac ccataatcat gtaaatgctc attttcttta ggacataaag agagccctag
  1621 ggtgctctga atctgtacat gttcttgtca taaaatgcat actgttgata caaaccactg
  1681 tgaacatttt ttatttgaga attttgtttc aaagggattg ctttttcctc tcattgtctt
  1741 gttatgtaca aactagtttt tatagctatc aacattagga gtaactttca accttgccag
  1801 catcactggt atgatgtata tttaattaaa gcacactttt ccccgaccgt atacttaaaa
  1861 tgacaaagcc attcttttaa atatttgtga ctctttccta aagccaaagt ttctgttgaa
  1921 ttatgttttg acacacccct aagtacaagg tggtatggtt gtatacacat gctgccttct
  1981 tggggattca aaaacaggtt tttgattttg aatagcaatt agtgatatag tgctgtttaa
  2041 gctactaacg ataaaaggta ataacatttt atacaatttc catatagtct attcattaag
  2101 taatcttttt acagttgcat caggcctgaa cccgtccatt cagaaagctt caaattatag
  2161 aaacaatact gttctatacg agtgaccgat tatgctttct ttggcctaca ttctttattc
  2221 tgcggtgaag ttgaggctta taagttaaaa caaaggaact aacttactgt ccaccagttt
  2281 atacagaact cacagtacct atgacttttt taaactaaga tctgttaaaa aagaaatctg
  2341 tttcaacaga tgaccgtgta caataccgtg tggtgaaaat gaattcagac ttattaaatg
  2401 atgaacttgt taaatcttct cagtgtctat ttatcagcac aatacacaca ggagaactgt
  2461 tgatggcata ttgaatagat tttcctgaat aaattgctct ggaaaccaca caaaaaaaaa
  2521 aaaaaa
Suz12 (accession No. NM_015355):
(SEQ ID NO: 105)
        1 ggtgagcggc ctccgaagcg gagcggggct ctgaggagac actttttttt tcctccctcc
      61 ttccctcctc tcctcctccc ttcccttccc ctctcctccc ctctctcctc cttcccccct
    121 cggtccgccg gagcctgctg gggcgagcgg ttggtattgc aggcgcttgc tctccggggc
    181 cgcccggcgg gtagctggcg gggggaggag gcaggaaccg cgatggcgcc tcagaagcac
    241 ggcggtgggg gagggggcgg ctcggggccc agcgcggggt ccgggggagg cggcttcggg
    301 ggttcggcgg cggtggcggc ggcgacggct tcgggcggca aatccggcgg cgggagctgt
    361 ggagggggtg gcagttactc ggcctcctcc tcctcctccg cggcggcagc ggcgggggct
    421 gcggtgttac cggtgaagaa gccgaaaatg gagcacgtcc aggctgacca cgagcttttc
    481 ctccaggcct ttgagaagcc aacacagatc tatagatttc ttcgaactcg gaatctcata
    541 gcaccaatat ttttgcacag aactcttact tacatgtctc atcgaaactc cagaacaaac
    601 atcaaaagga aaacatttaa agttgatgat atgttatcaa aagtagagaa aatgaaagga
    661 gagcaagaat ctcatagctt gtcagctcat ttgcagctta cgtttactgg tttcttccac
    721 aaaaatgata agccatcacc aaactcagaa aatgaacaaa attctgttac cctggaagtc
    781 ctgcttgtga aagtttgcca caaaaaaaga aaggatgtaa gttgtccaat aaggcaagtt
    841 cccacaggta aaaagcaggt gcctttgaat cctgacctca atcaaacaaa acccggaaat
    901 ttcccgtccc ttgcagtttc cagtaatgaa tttgaaccta gtaacagcca tatggtgaag
    961 tcttactcgt tgctatttag agtgactcgt ccaggaagaa gagagtttaa tggaatgatt
  1021 aatggagaaa ccaatgaaaa tattgatgtc aatgaagagc ttccagccag aagaaaacga
  1081 aatcgtgagg atggggaaaa gacatttgtt gcacaaatga cagtatttga taaaaacagg
  1141 cgcttacagc ttttagatgg ggaatatgaa gtagccatgc aggaaatgga agaatgtcca
  1201 ataagcaaga aaagagcaac atgggagact attcttgatg ggaagaggct gcctccattc
  1261 gaaacatttt ctcagggacc tacgttgcag ttcactcttc gttggacagg agagaccaat
  1321 gataaatcta cggctcctat tgccaaacct cttgccacta gaaattcaga gagtctccat
  1381 caggaaaaca agcctggttc agttaaacct actcaaacta ttgctgttaa agaatcattg
  1441 actacagatc tacaaacaag aaaagaaaag gatactccaa atgaaaaccg acaaaaatta
  1501 agaatatttt atcagtttct ctataacaac aatacaaggc aacaaactga agcaagagat
  1561 gacctgcatt gcccttggtg tactctgaac tgccgcaaac tttatagttt actcaagcat
  1621 cttaaactct gccatagcag atttatcttc aactatgttt atcatccaaa aggtgctagg
  1681 atagatgttt ctatcaatga gtgttatgat ggctcctatg caggaaatcc tcaggatatt
  1741 catcgccaac ctggatttgc ttttagtcgc aacggaccag ttaagagaac acctatcaca
  1801 catattcttg tgtgcaggcc aaaacgaaca aaagcaagca tgtctgaatt tcttgaatct
  1861 gaagatgggg aagtagaaca gcaaagaaca tatagtagtg gccacaatcg tctgtatttc
  1921 catagtgata cctgcttacc tctccgtcca caagaaatgg aagtagatag tgaagatgaa
  1981 aaggatcctg aatggctaag agaaaaaacc attacacaaa ttgaagagtt ttctgatgtt
  2041 aatgaaggag agaaagaagt gatgaaactc tggaatctcc atgtcatgaa gcatgggttt
  2101 attgctgaca atcaaatgaa tcatgcctgt atgctgtttg tagaaaatta tggacagaaa
  2161 ataattaaga agaatttatg tcgaaacttc atgcttcatc tagtcagcat gcatgacttt
  2221 aatcttatta gcataatgtc aatagataaa gctgttacca agctccgtga aatgcagcaa
  2281 aaattagaaa agggggaatc tgcttcccct gcaaacgaag aaataactga agaacaaaat
  2341 gggacagcaa atggatttag tgaaattaac tcaaaagaga aagctttgga aacagatagt
  2401 gtctcagggg tttcaaaaca gagcaaaaaa caaaaactct gaaaagctct aaccccatgt
  2461 tatggacaaa cactgaaatt acattttagg gaattcatcc tctaagaatt atgtttttgt
  2521 ttttaatcat atgttccaaa caggcactgt tagatgaagt aaatgatttc aacaaggata
  2581 tttgtatcag ggttctactt cacttcatta tgcagcatta catgtatatc acttttattg
  2641 atgtcattaa aacattctgt actttaagca tgaaaagcaa tatttcaaag tatttttaaa
  2701 ctcaacaaat gtcatcaaat atgttgaatt gatctagaaa ttatttcata tataaatcag
  2761 aatttttttg catttatgaa cggctgtttt tctactttgt aattgtgaga cattttcttg
  2821 gggagggaaa attggaatgg ttcccttttt tagaaattga agtggtcttc atatgtcaac
  2881 tacagaaaag gaaaaaaata gaaattgaag gatttttatg aaattatatt gcattactat
  2941 ttgcagtcaa actttgatcc ttgtttttga aatcatttgt caattcggaa tgaaaaatta
  3001 taatgtaatt ttacattaca taagttcctt ttacaattaa aaaatagcac ttcttcatct
  3061 tatgcctgtt tgagaagata ttaaattttc acattgttga cagtgaaatg ctatgttggt
  3121 ttataagatt acagaccatt tgttttcatg tggataattt tagtgcattg ctcacccggt
  3181 atgttttttt tttttaactt gaacattttg cttgttttgt ttttcttttt taattagata
  3241 atcacacgga aaattaagct gttcatatct ttaaattagg attgcaaacc aaggaaagaa
  3301 cgcatttgag attttaagat gtcacttata aggggagaag tgttcttaaa aagtcaacca
  3361 gaaaactgtt atgcctttta tttgtttgca aggatgtctt tgtaatgtgt ttcatgaata
  3421 gaatatccaa tagagataag ctgacttgaa tcattttgag caattttgcc ctgtgttata
  3481 tgtgtttcac gcacatattt gcagttggat tttctccaac agaaagtgga ttcactactg
  3541 gcacattaac aagcaccaat aggtttttat tccaactccg agcactgtgg ttgagtaaca
  3601 tcacctcaat tttttattat ccttaaagat attgcatttt catattcttt atttataaag
  3661 gatcaatgct gctgtaaata caggtatttt taattttaaa atttcattcc accaccatca
  3721 gatgcagttc cctattttgt ttaatgaagg gatatataag ctttctaatg gtgtcttcag
  3781 aaatttataa aatgtaaata ctgatttgac tggtctttaa gatgtgttta actgtgaggc
  3841 tatttaacga atagtgtgga tgtgatttgt catccagtat taagttctta gtcattgatt
  3901 tttgtgttta aaaaaaaata ggaaagaggg aaactgcagc tttcattaca gattccttga
  3961 ttggtaagct ctccaaatga tgagttctag taaactctga tttttgcctc tggatagtag
  4021 atctcgagcg tttatctcgg gctttaattt gctaaagctg tgcacatatg taaaaaaaaa
  4081 aaaaaaaaga ttattttagg ggagatgtag gtgtagaatt attgcttatg tcatttctta
  4141 agcagttatg ctcttaatgc ttaaaagaag gctagcattg tttgcacaaa aagttggtga
  4201 ttcccacccc aaatagtaat aaaattactt ctgttgagta aactttttat gtcatcgtaa
  4261 aagctgaaaa aatccctttg tttctattta taaaaaaagt gcttttctat atgtaccctt
  4321 gataacagat tttgaagaaa tcctgtaaga tgataaagca tttgaatggt acagtagatg
  4381 taaaaaaaat tcagtttaaa agaacatttg tttttacatt aaatgtttat ttgaaatcaa
  4441 atgattttgt acataaagtt caataatata aaagctg
EED (accession No. NM_003797):
(SEQ ID NO: 106)
        1 cgctttgaaa tccaccctgg gattcggaaa ccgggtagaa aactacctgg ttcagcaaac
      61 gagaattcaa acagaggagg ggcttggagg aggcgggttt cgacgaaccc agcgcaagag
    121 tacgccacgg cgcctgcgca tcccctgacg ggtactttcc attcgccaga tgggggaagc
    181 cagggggaag caggttactg tttttgcatt tctatcttca aggaagaatt aggttatgaa
    241 tagttccgtg aatagtcagg aagcgctgtc ctccaagttc aagattaagg aaacgtggca
    301 tgcacagcta aagcaagagg tgacgtcttg tatcttcccc cgtttcctgg gacattggtg
    361 gtgtagccca ttccacagac tttcgctccc tagcagcggg tcggagatcg aaggaacggg
    421 ccaattgcgg ctgaaacgtc tttggaagga ggaagggggt gagggagcat ccctttgagt
    481 ttcgcctctt ctcgaggcgg tggtgggaag ggagacatac ttaatactgc cctcttaatc
    541 caacggacct tacatcgtgt agactgccgg gagggcggcg ggaaaagggc aagacgggag
    601 ttggggaagg gaaggagcca ggaagccgcg cgggagggcg cgcgcgcgcg cccctttttc
    661 agcagtgtgg cggggtcgca cgcacgcccg cctcggcggc tgggcgcgat ttgcgacagt
    721 ggggggggcg gtggaggtgg cggcggcagc ggcaactttg cggcaagctc gggccgggct
    781 tgcttgacgg cggtgtggcg gaggccccgc cccaggcggc aggaacctgg agggaggcgg
    841 aggaatatgt ccgagaggga agtgtcgact gcgccggcgg gaacagacat gcctgcggcc
    901 aagaagcaga agctgagcag tgacgagaac agcaatccag acctctctgg agacgagaat
    961 gatgacgctg tcagtataga aagtggtaca aacactgaac gccctgatac acctacaaac
  1021 acgccaaatg cacctggaag gaaaagttgg ggaaagggaa aatggaagtc aaagaaatgc
  1081 aaatattctt tcaaatgtgt aaatagtctc aaggaagatc ataaccaacc attgtttgga
  1141 gttcagttta actggcacag taaagaagga gatccattag tgtttgcaac tgtaggaagc
  1201 aacagagtta ccttgtatga atgtcattca caaggagaaa tccggttgtt gcaatcttac
  1261 gtggatgctg atgctgatga aaacttttac acttgtgcat ggacctatga tagcaatacg
  1321 agccatcctc tgctggctgt agctggatct agaggcataa ttaggataat aaatcctata
  1381 acaatgcagt gtataaagca ctatgttggc catggaaatg ctatcaatga gctgaaattc
  1441 catccaagag atccaaatct tctcctgtca gtaagtaaag atcatgcttt acgattatgg
  1501 aatatccaga cggacactct ggtggcaata tttggaggcg tagaagggca cagagatgaa
  1561 gttctaagtg ctgattatga tcttttgggt gaaaaaataa tgtcctgtgg tatggatcat
  1621 tctcttaaac tttggaggat caattcaaag agaatgatga atgcaattaa ggaatcttat
  1681 gattataatc caaataaaac taacaggcca tttatttctc agaaaatcca ttttcctgat
  1741 ttttctacca gagacataca taggaattat gttgattgtg tgcgatggtt aggcgatttg
  1801 atactttcta agtcttgtga aaatgccatt gtgtgctgga aacctggcaa gatggaagat
  1861 gatatagata aaattaaacc cagtgaatct aatgtgacta ttcttgggcg atttgattac
  1921 agccagtgtg acatttggta catgaggttt tctatggatt tctggcaaaa gatgcttgca
  1981 ttgggcaatc aagttggcaa actttatgtt tgggatttag aagtagaaga tcctcataaa
  2041 gccaaatgta caacactgac tcatcataaa tgtggtgctg ctattcgaca aaccagtttt
  2101 agcagggata gcagcattct tatagctgtt tgtgatgatg ccagtatttg gcgctgggat
  2161 cgacttcgat aaaatacttt tgcctaatca aaattagagt gtgtttgttg tctgtgtaaa
  2221 atagaattaa tgtatcttgc tagtaagggc acgtagagca tttagagttg tctttcagca
  2281 ttcaatcagg ctgagctgaa tgtagtgatg tttacattgt ttacattctt tgtactgtct
  2341 tcctgctcag actctactgc ttttaataaa aatttatttt tgtaaagctg tgtgtttagt
  2401 tactttcatt gtggtgaaaa aaagttaaaa gtaataaaat tatgccttat ctttttaaaa
  2461 aaaaaaaaaa aaaaaa
EZH1 (accession No. NM_001991):
(SEQ ID NO: 107)
        1 gcgcatgcgt cctagcagcg ggacccgcgg ctcgggatgg aggctggaca cctgttctgc
      61 tgttgtgtcc tgccattctc ctgaagaaca gaggcacact gtaaaaccca acacttcccc
    121 ttgcattcta taagattaca gcaagatgga aataccaaat ccccctacct ccaaatgtat
    181 cacttactgg aaaagaaaag tgaaatctga atacatgcga cttcgacaac ttaaacggct
    241 tcaggcaaat atgggtgcaa aggctttgta tgtggcaaat tttgcaaagg ttcaagaaaa
    301 aacccagatc ctcaatgaag aatggaagaa gcttcgtgtc caacctgttc agtcaatgaa
    361 gcctgtgagt ggacaccctt ttctcaaaaa gtgtaccata gagagcattt tcccgggatt
    421 tgcaagccaa catatgttaa tgaggtcact gaacacagtt gcattggttc ccatcatgta
    481 ttcctggtcc cctctccaac agaactttat ggtagaagat gagacggttt tgtgcaatat
    541 tccctacatg ggagatgaag tgaaagaaga agatgagact tttattgagg agctgatcaa
    601 taactatgat gggaaagtcc atggtgaaga agagatgatc cctggatccg ttctgattag
    661 tgatgctgtt tttctggagt tggtcgatgc cctgaatcag tactcagatg aggaggagga
    721 agggcacaat gacacctcag atggaaagca ggatgacagc aaagaagatc tgccagtaac
    781 aagaaagaga aagcgacatg ctattgaagg caacaaaaag agttccaaga aacagttccc
    841 aaatgacatg atcttcagtg caattgcctc aatgttccct gagaatggtg tcccagatga
    901 catgaaggag aggtatcgag aactaacaga gatgtcagac cccaatgcac ttccccctca
    961 gtgcacaccc aacatcgatg gccccaatgc caagtctgtg cagcgggagc aatctctgca
  1021 ctccttccac acactttttt gccggcgctg ctttaaatac gactgcttcc ttcacccttt
  1081 tcatgccacc cctaatgtat ataaacgcaa gaataaagaa atcaagattg aaccagaacc
  1141 atgtggcaca gactgcttcc ttttgctgga aggagcaaag gagtatgcca tgctccacaa
  1201 cccccgctcc aagtgctctg gtcgtcgccg gagaaggcac cacatagtca gtgcttcctg
  1261 ctccaatgcc tcagcctctg ctgtggctga gactaaagaa ggagacagtg acagggacac
  1321 aggcaatgac tgggcctcca gttcttcaga ggctaactct cgctgtcaga ctcccacaaa
  1381 acagaaggct agtccagccc cacctcaact ctgcgtagtg gaagcaccct cggagcctgt
  1441 ggaatggact ggggctgaag aatctctttt tcgagtcttc catggcacct acttcaacaa
  1501 cttctgttca atagccaggc ttctggggac caagacgtgc aagcaggtct ttcagtttgc
  1561 agtcaaagaa tcacttatcc tgaagctgcc aacagatgag ctcatgaacc cctcacagaa
  1621 gaagaaaaga aagcacagat tgtgggctgc acactgcagg aagattcagc tgaagaaaga
  1681 taactcttcc acacaagtgt acaactacca accctgcgac cacccagacc gcccctgtga
  1741 cagcacctgc ccctgcatca tgactcagaa tttctgtgag aagttctgcc agtgcaaccc
  1801 agactgtcag aatcgtttcc ctggctgtcg ctgtaagacc cagtgcaata ccaagcaatg
  1861 tccttgctat ctggcagtgc gagaatgtga ccctgacctg tgtctcacct gtggggcctc
  1921 agagcactgg gactgcaagg tggtttcctg taaaaactgc agcatccagc gtggacttaa
  1981 gaagcacctg ctgctggccc cctctgatgt ggccggatgg ggcaccttca taaaggagtc
  2041 tgtgcagaag aacgaattca tttctgaata ctgtggtgag ctcatctctc aggatgaggc
  2101 tgatcgacgc ggaaaggtct atgacaaata catgtccagc ttcctcttca acctcaataa
  2161 tgattttgta gtggatgcta ctcggaaagg aaacaaaatt cgatttgcaa atcattcagt
  2221 gaatcccaac tgttatgcca aagtggtcat ggtgaatgga gaccatcgga ttgggatctt
  2281 tgccaagagg gcaattcaag ctggcgaaga gctcttcttt gattacaggt acagccaagc
  2341 tgatgctctc aagtacgtgg ggatcgagag ggagaccgac gtcctttagc cctcccaggc
  2401 cccacggcag cacttatggt agcggcactg tcttggcttt cgtgctcaca ccactgctgc
  2461 tcgagtctcc tgcactgtgt ctcccacact gagaaacccc ccaacccact ccctctgtag
  2521 tgaggcctct gccatgtcca gagggcacaa aactgtctca atgagagggg agacagaggc
  2581 agctagggct tggtctccca ggacagagag ttacagaaat gggagactgt ttctctggcc
  2641 tcagaagaag cgagcacagg ctggggtgga tgacttatgc gtgatttcgt gtcggctccc
  2701 caggctgtgg cctcaggaat caacttaggc agttcccaac aagcgctagc ctgtaattgt
  2761 agctttccac atcaagagtc cttatgttat tgggatgcag gcaaacctct gtggtcctaa
  2821 gacctggaga ggacaggcta agtgaagtgt ggtccctgga gcctacaagt ggtctgggtt
  2881 agaggcgagc ctggcaggca gcacagactg aactcagagg tagacaggtc accttactac
  2941 ctcctccctc gtggcagggc tcaaactgaa agagtgtggg ttctaagtac aggcattcaa
  3001 ggctggggga aggaaagcta cgccatcctt ccttagccag agagggagaa ccagccagat
  3061 gatagtagtt aaactgctaa gcttgggccc aggaggcttt gagaaagcct tctctgtgta
  3121 ctctggagat agatggagaa gtgttttcag attcctggga acagacacca gtgctccagc
  3181 tcctccaaag ttctggctta gcagctgcag gcaagcatta tgctgctatt gaagaagcat
  3241 taggggtatg cctggcaggt gtgagcatcc tggctcgctg gatttgtggg tgttttcagg
  3301 ccttccattc cccatagagg caaggcccaa tggccagtgt tgcttatcgc ttcagggtag
  3361 gtgggcacag gcttggacta gagaggagaa agattggtgt aatctgcttt cctgtctgta
  3421 gtgcctgctg tttggaaagg gtgagttaga atatgttcca aggttggtga ggggctaaat
  3481 tgcacgcgtt taggctggca ccccgtgtgc agggcacact ggcagagggt atctgaagtg
  3541 ggagaagaag caggtagacc acctgtccca ggctgtggtg ccaccctctc tggcattcat
  3601 gcagagcaaa gcactttaac catttctttt aaaaggtcta tagattgggg tagagtttgg
  3661 cctaaggtct ctagggtccc tgcctaaatc ccactcctga gggaggggga agaagagagg
  3721 gtgggagatt ctcctccagt cctgtctcat ctcctgggag aggcagacga gtgagtttca
  3781 cacagaagaa tttcatgtga atggggccag caagagctgc cctgtgtcca tggtgggtgt
  3841 gccgggctgg ctgggaacaa ggagcagtat gttgagtaga aagggtgtgg gcgggtatag
  3901 attggcctgg gagtgttaca gtagggagca ggcttctccc ttctttctgg gactcagagc
  3961 cccgcttctt cccactccac ttgttgtccc atgaaggaag aagtggggtt cctcctgacc
  4021 cagctgcctc ttacggtttg gtatgggaca tgcacacaca ctcacatgct ctcactcacc
  4081 acactggagg gcacacacgt accccgcacc cagcaactcc tgacagaaag ctcctcccac
  4141 ccaaatgggc caggccccag catgatcctg aaatctgcat ccgccgtggt ttgtattcat
  4201 tgtgcatatc agggataccc tcaagctgga ctgtgggttc caaattactc atagaggaga
  4261 aaaccagaga aagatgaaga ggaggagtta ggtctatttg aaatgccagg ggctcgctgt
  4321 gaggaatagg tgaaaaaaaa cttttcacca gcctttgaga gactagactg accccaccct
  4381 tccttcagtg agcagaatca ctgtggtcag tctcctgtcc cagcttcagt tcatgaatac
  4441 tcctgttcct ccagtttccc atcctttgtc cctgctgtcc cccactttta aagatgggtc
  4501 tcaacccctc cccaccacgt catgatggat ggggcaaggt ggtggggact aggggagcct
  4561 ggtatacatg cggcttcatt gccaataaat ttcatgcact ttaaagtcct gtggcttgtg
  4621 acctcttaat aaagtgttag aatccaaaaa aaaa
RbAp48 (accession No. NM_005610):
(SEQ ID NO: 108)
        1 gctcccattg gctgatgttg gcgcgaaggt gcgcgagtca gccctcgcgc tgggggcgca
      61 ggaaacaata gaggccgcgc gcacagagcg agctcttgca gcctccccgc ccctcccgca
    121 acgctcgacc ccaggattcc cccggctcgc ctgcccgcca tggccgacaa ggaagcagcc
    181 ttcgacgacg cagtggaaga acgagtgatc aacgaggaat acaaaatatg gaaaaagaac
    241 accccttttc tttatgattt ggtgatgacc catgctctgg agtggcccag cctaactgcc
    301 cagtggcttc cagatgtaac cagaccagaa gggaaagatt tcagcattca tcgacttgtc
    361 ctggggacac acacatcgga tgaacaaaac catcttgtta tagccagtgt gcagctccct
    421 aatgatgatg ctcagtttga tgcgtcacac tacgacagtg agaaaggaga atttggaggt
    481 tttggttcag ttagtggaaa aattgaaata gaaatcaaga tcaaccatga aggagaagta
    541 aacagggccc gttatatgcc ccagaaccct tgtatcatcg caacaaagac tccttccagt
    601 gatgttcttg tttttgacta tacaaaacat ccttctaaac cagatccttc tggagagtgc
    661 aacccagact tgcgtctccg tggacatcag aaggaaggct atgggctttc ttggaaccca
    721 aatctcagtg ggcacttact tagtgcttca gatgaccata ccatctgcct gtgggacatc
    781 agtgccgttc caaaggaggg aaaagtggta gatgcgaaga ccatctttac agggcatacg
    841 gcagtagtag aagatgtttc ctggcatcta ctccatgagt ctctgtttgg gtcagttgct
    901 gatgatcaga aacttatgat ttgggatact cgttcaaaca atacttccaa accaagccac
    961 tcagttgatg ctcacactgc tgaagtgaac tgcctttctt tcaatcctta tagtgagttc
  1021 attcttgcca caggatcagc tgacaagact gttgccttgt gggatctgag aaatctgaaa
  1081 cttaagttgc attcctttga gtcacataag gatgaaatat tccaggttca gtggtcacct
  1141 cacaatgaga ctattttagc ttccagtggt actgatcgca gactgaatgt ctgggattta
  1201 agtaaaattg gagaggaaca atccccagaa gatgcagaag acgggccacc agagttgttg
  1261 tttattcatg gtggtcatac tgccaagata tctgatttct cctggaatcc caatgaacct
  1321 tgggtgattt gttctgtatc agaagacaat atcatgcaag tgtggcaaat ggcagagaac
  1381 atttataatg atgaagaccc tgaaggaagc gtggatccag aaggacaagg gtcctagata
  1441 tgtctttact tgttgtgatt ttagactccc cttttttctt ctcaaccctg agagtgattt
  1501 aacactggtt ttgagacaga ctttattcag ctatccctct atataatagg taccaccgat
  1561 aatgctatta gcccaaaccg tgggtgtttt ctaaatatta ataggggggc ttgattcaac
  1621 aaagccacag acttaacgtt gaaattttct tcaggaattt tctagtaacc caggtctaaa
  1681 gtagctacag aaaggggaat attatgtgtg attatttttc ttcttatgct atatccccaa
  1741 gtttttcaga ctcatttaag taaaggctag agtgagtaag gaatagagcc aaatgaggta
  1801 ggtgtctgag ccatgaagta taaatactga aagatgtcac ttttattcag gaaatagggg
  1861 gagattcaag tcatatagat tcctactcga aaatcttgac acctgacttt ccaggatgca
  1921 cattttcata cgtagaccag tttcctcttg gtttcttcag ttaagtcaaa acaacacgtt
  1981 cctctttccc catatattca tatatttttg ctcgttagtg tatttcttga gctgttttca
  2041 tgttgtttat ttcctgtctg tgaaatggtg tttttttttt tgttgttggt tttttttttt
  2101 ttttttttaa cttgggacca ccaagttgta aagatgtatg tttttacctg acagttatac
  2161 cacaggtaga ctgtcaagtt gagaagagtg aatcaataac ttgtatttgt tttaaaaatt
  2221 aaattaatcc ttgataagag ttgctttttt tttttaggag ttagtccttg accactagtt
  2281 tgatgccatc tccattttgg gtgacctgtt tcaccagcag gcctgttact ctccatgact
  2341 aactgtgtaa gtgcttaaaa tggaataaat tgcttttcta cataacccca tgctgatggg
  2401 ttttatttag tataaaacat ccatcaaaca ccagtctctg gcttctagaa gagtccttca
  2461 gatgacagtt gttgtccatg gtctttgact atcaagagca gaattaaatg taatagtccc
  2521 agagctgtag aaaagaactt tactccttcc cagggaaagt gaaagacata aaacactgaa
  2581 tcagaggtgg cacagattag tctttgataa ggtaacgttt ctttgaagtc tatctgtaga
  2641 gaactacatg gacttccaag agtgtcaaag gcagtgtggt agagagaatt taaggcaaga
  2701 tttaaatttg gaaaaggtgc ttgaaccttt tctcagaggt tttatttccc cagtatgttt
  2761 ttcactgggg cctttactta ggttagaaat aataggcttt gaaggcctct atcaccagat
  2821 gcaataacca gataaaattc ctgttttttc ccaatcgctt agttttttgt tgttgttgtt
  2881 ttttaactga gtagatcatt ctgacccaga actactttca tgaggtaaga tctttgggaa
  2941 aatctgaata gcgttaacca ttagattcaa atctcaaatg gtttcttttc aagtctagtt
  3001 gttttagagt atagtgagaa ataccttgac acaattttaa gagtaaacta tatgggtcag
  3061 catatccttg aacaaaaagt agactttgta aaagtattca tttaaattct aacactcgtg
  3121 gcacaaaaga atggaaattg taaacccatg taatggaaat tggctatctt tttgacccca
  3181 catgtgcccc tcaaaaatgt ttttggtttg ggtcaacaca aggcaagata cattctttaa
  3241 aatactccca gatgtgtcca tacattcatc cttcactcag tgcatatgtg agggttgttg
  3301 ctggaagaca ggaggctcat ctttcctttc cttggtgcat tgagatcagt atcaacagca
  3361 gatgaaatag aatccagcaa agagttgaca tgttctgcct ccggccaact ctagaatctt
  3421 tttaagcagg tcagccagta tttgcaactt ccacaggatg aattgcttgc caagtttctg
  3481 gcactcttgt ctggttggaa gagtacatcc aaagggtact tagtgatcct ttgctaagaa
  3541 gttttttgct gtttccgggt tacagatttg gccatatatt tctaaacagc ccctgtaaag
  3601 ttgaaagaaa aagtttataa cagtgaactt ctgaggttta gttactgcag gctttgttga
  3661 gaagagattg ttacagtgtg atttatggat gatcagggat gactttcccc tagcaaatat
  3721 ttggatgcct cctgtttgtc aaatagaatg aatggtgatg gtgatgggag ggatagttaa
  3781 acgttttctc tgctaggtta acttcttaca ggtataatta caatgcctga aattctgtag
  3841 tttcatttct ttggattagt cgttgtcttt tccagattgt acacaatctg atcaacacaa
  3901 aggtagttag tagatcatta acctcaattg caaggttata atttctcaaa cattaagcat
  3961 attatcagtc atgtggattc aaacacagta taagaaaatc ctcaaggctg ggtgtgatgg
  4021 cttatgcctg taatcccagc tacttagcag gctgaggcag gagtattgtt tgaacccagg
  4081 aggcagagtt gcagtgagcc aagatcgtgc cactactcca gcctgggcaa aatagcaaga
  4141 cccgaccccc catctctact aaaaagaaat ttaaaaaaat aaaatcctag aaaattagaa
  4201 aaagcaacaa tagttacttg tgggccaggc gcggtggttc acgcctgtaa tcccagcact
  4261 ttgagaggct gaggcacgtg gatcacaagg tcaggagttc aagaccagcc tggccaagat
  4321 ggtgaaaccc cgtttctact aaaaatacaa aaactagctg gccgtggtgg catgctcctg
  4381 tagtcccagc tactcaggag gctgaggcag gaaaatcact tgaacccagg aggtggaggt
  4441 tgcagtgaac tgagaccgtg ccactgcact ccagcctggg tggcagagcg agactgtctc
  4501 aaaaaaaaaa gaaagttgtg aatttgatgt aagcttagga aatgaataaa atttataggc
  4561 atctgtataa tgtacaactt gacacggact ttcttttatc cttagtttct ttcacggact
  4621 ctagaacttt tatcagaata tactggtaaa acattggggg agggatcctg agtaggtgat
  4681 tggtcagaaa gatgccttca gttttgtcag tgtctaaaag ttaagtctgt ttaggccaag
  4741 catggtggct cacacctgaa atcccagcac tctgggaggc cgaggcaagt ggatcacaag
  4801 gtcaggagat gagaccatct tagccaacat ggtgaaaccc cgtctctact aaaatacaaa
  4861 aaaattagcc aggcgtggtg gtgcgtgcct ataatcccag ctacttggga ggctgaggca
  4921 ggggaatcgc ttgaacccgg gaggcagagg tcacgccact gcactccagc ctggcaacag
  4981 agcaagactc cgtctcaaaa aaaaaaaaaa aaaaaaagag taagtctgtg taacatgaac
  5041 atctctgctt ccacccaaaa ccacagcctt tgaatattat ataaggaact taatggatag
  5101 atatgtttat tatttttgat agcacaactg ctttctctgc tattataagg aaaactgaga
  5161 atagcaggtg ggtagggtag gatgaggaaa caagatgccc aaagcctaga tgccacagaa
  5221 ttcatggtga taatcagggc atattttgag tcctactaga aacaaacatt ccaaatgaac
  5281 tctgaatgcc tgactcaggc gttttggagg tttgggttat ccccttgtca ttaggcacac
  5341 aagggttttt tgttgttttt gttttttggg tttttgtttt gttttgaggc agtctcactc
  5401 tgttgtacaa gctggagtgc tgtattgtga tcttgactca ctgcaacctc tgcctcctgg
  5461 ttcaagcgat tctcctgcct tggcctcctg agtagctggg attataagtg cctgccacta
  5521 tgcccggcta atttttgtat ttttagtgga gatggagttt tgccatgttg gccaggctgg
  5581 tcttgaactc ctgactccag gtgatccacc ctcctcagcc tcccaaagtg ctaggattac
  5641 aggcgtgagc caccccgtcc ggcctgtttt taaggcatta attagtattg ttaggaaagc
  5701 agtaacaatg caaacaccac tcttctcttc acaaagatca ccttgagact gtgtctccat
  5761 tccacctgcc tgagaagtgg gagcatcagc ctgttccagg ctcttgggta gtagcatagc
  5821 cctttaaaaa gagagagcca ttttccatgt gtttttggat aagcacaatt tgaaaatcat
  5881 ttcccaaatc ctctttttgt ttttgattct aaggtaaaat tttccctaag ccctcccacc
  5941 atcccctcag ccagtattag atgagatttg tatagcagca gaaactgact tataagtaga
  6001 gagctcttca gcaagactga gccttagctg ttccatctct ttgttcttct gttgctggag
  6061 ttgcacccca tttcttaact gcctctggcg ttcttccatt tcctccagct gttcctgcat
  6121 gagatggcca agaacatttc taatgagcca aacaataaaa actcacattg tccactctta
  6181 cttataaaac acttttttgt tcattgttta atcttgatag cagtattgag gctggtattt
  6241 atatgatagg ttatgaaaca ggttcaaaga agttgtgtct tggaaaaaaa gtgacaatgc
  6301 ttttgaaaat gatgacgaaa aaggcatctt gtctgttaac cacagcttgc tttaatagaa
  6361 tcctgggagg gtgattggga ctttttagta ttacaacctt agtgtcattg aggaggattt
  6421 tggtctagtt agtgggctga gtttcatata cctctccctc catgtgcagg tttgttaaga
  6481 taattggtag tttttaataa tataaaatac ttaagttgaa atacaaaagt gtggcaacaa
  6541 ttattaaata ttggctagaa ttctaggaga gttacacaac tagtggaagt ccatgtttag
  6601 aaaataaatg gcttgtttaa ggaaaagttt ttgtgtccaa agctccttaa agtcagagag
  6661 atttctacct ggtacttaac atcatatgga aattgatgct ttagtgaggg tgttggctat
  6721 cctattgcta atttcctgca tccttttttc ttctttattt ttgtatagag acagggtctc
  6781 gctatgttgc ccaggctggt cttgttcctg ggctcaagca gtcctcccgc ctcggtctcc
  6841 caaagtgctg ggattacagg tgtgagccac tgtgcccagc ttatcctttt ttcattacac
  6901 aaaaagactg aatttggtta gttctaagtt ggaagataaa gatggtatgc acaggaggcc
  6961 cttgggagcc ctcagataac tttctcattc ttccagaatc aggctgggat gcattctgta
  7021 aattttccct gcctaggatg tatacctgag gaataaggta aggaagatgt cagcaagtca
  7081 gtctctggtt tacctgctag ctggcatgga tccttaagga agcaggaggg agttgggaag
  7141 agaggaaggg gtgaagttgg tatcttttaa agcgagagtg attttacctc agattttgaa
  7201 gaatactaag gaatccagtt gttggggtac atgctattat tagaaggatc tagataattt
  7261 gtcctctgag tcatacttga cattgtacct gtggcacatc aatccgcact gtttgatact
  7321 ctggctgaat ctcagctttc accaacattg tcaaaggacc ttttttagtg cccagccatg
  7381 cctaagagtg tgtcatctga agagggaagc atctgcatac tgctgtcctg attgctcagt
  7441 cctcactacc taccagaccc gttggtaagg tacaaaagta catgcttgga aaagcagtct
  7501 gcaccaccag tgataagctg tgacagagtg gaacagcctc aatgaaatga aggaaggatt
  7561 gctacagtgg cattaaggat ggtctcttaa tcctgtgtta accactagat taactttaca
  7621 atcaactcaa aatccttcaa aggctttcca ctttctttag tggcattcag accccctcta
  7681 gtttgacccc tacctccaac ttgaacctct gttactcttc cgtatgaaca ttttcctcta
  7741 gccctggact actagtaccg aagtcactag tcacatagga ctcatttgaa atatgactag
  7801 tctcaattga gatgtaatgt aagtgtaaaa tacacagcag atttctaaga cagcacacaa
  7861 aatgtaaaat atgtcaaaaa tatttgatac tgattacatg ttgaaatata tgtgttgggt
  7921 taaataaaat gcattaaagt taa
Jarid2 (accession No. NM_004973):
(SEQ ID NO: 109)
        1 tggatagcct ctctctcatt ggttaggggg cttggaaaaa agagactcgg cgagccctcg
      61 ctgtggtgct gccgccgccg ccgccgccgc cgctggagtt gactcttctg ctcgcactgc
    121 tgctgcagca caaacgtgac ttccaacatt ttttatttat ctttcccttt tcttttccaa
    181 gatgtaacta cggatcagac actaaggacc ttcacgtttc gctgatgtag tttttggagg
    241 aaaaaggggg gggagtgaag ggcgtcggtt tttttttgtg tgtgtgtgta tgtgtttcgg
    301 gggaaatttt ccattatgag tgttttacta aagtgaattt ttttttgttt gcttcgttcg
    361 tctttggctc tttttttttc cttcccaatt tcggatttat ttcaaggcga atctggcttt
    421 gggggaagag gaagaaaagt cggattacaa gatcaaccac caccaacaac aataaaaacc
    481 accaggatat ttttttgcaa atttctgacg gctttaaatt catgaagcaa ttgtcccctt
    541 ttgcaatcag catttggatc tcagaatgag caaggaaaga cccaagagga atatcattca
    601 gaagaaatac gatgacagtg atgggattcc gtggtcagaa gaacgggtgg tacgtaaagt
    661 cctttatttg tctctgaagg agttcaagaa ttcccagaag aggcagcatg cggaaggcat
    721 tgctgggagc ctgaaaactg tgaatgggct ccttggtaat gaccagtcta agggattagg
    781 accagcatca gaacagtcag agaatgaaaa ggacgatgca tcccaagtgt cctccactag
    841 caacgatgtt agttcttcag attttgaaga agggccgtcg aggaaaaggc ccaggctgca
    901 agcacaaagg aagtttgctc agtctcagcc gaatagtccc agcacaactc cagtaaagat
    961 agtggagcca ttgctacccc ctccagctac tcagatatca gacctctcta aaaggaagcc
  1021 taagacagaa gattttctta cctttctctg ccttcgaggt tctcctgcgc tgcccaacag
  1081 catggtgtat tttggaagct ctcaggatga ggaggaagtc gaggaggaag atgatgagac
  1141 agaagacgtc aaaacagcca ccaacaatgc ttcatcttca tgccagtcga cccccaggaa
  1201 aggaaaaacc cacaaacatg ttcacaacgg gcatgttttc aatggttcca gcaggtcaac
  1261 acgggagaag gaacctgttc aaaaacacaa aagcaaagag gccactcccg caaaggagaa
  1321 gcacagcgat caccgggctg acagccgccg ggagcaggct tcagctaacc accccgcagc
  1381 ggccccctcc acgggttcct cggccaaggg gcttgctgcc acccatcacc acccccctct
  1441 gcatcggtcg gctcaggact tacggaaaca ggtttctaag gtaaacggag tcactcgaat
  1501 gtcatctctg ggtgcaggtg taaccagtgc caaaaagatg cgcgaggtca gaccttcacc
  1561 atccaaaact gtgaagtaca ctgccacggt gacgaagggg gctgtcacat acaccaaagc
  1621 caagagagaa ctggtcaagg acaccaaacc caatcaccac aagcccagtt ccgctgtcaa
  1681 ccacacaatc tcagggaaaa ctgaaagtag caatgcaaaa acccgcaaac aggtgctatc
  1741 cctcgggggg gcgtccaagt ccactgggcc cgccgtcaat ggcctcaagg tcagtggcag
  1801 gttgaaccca aagtcatgca ctaaggaggt gggggggcgg cagctgcggg agggcctgca
  1861 gctgcgggag gggctgcgga actccaagag gagactggaa gaggcacacc aggcggagaa
  1921 gccgcagtcg ccccccaaga agatgaaagg ggcggctggc cccgccgaag gccctggcaa
  1981 gaaggccccg gccgagagag gtctgctgaa cggacacgtg aagaaggaag tgccggagcg
  2041 cagtctggag aggaatcggc cgaagcgggc cacggccggg aagagcacgc caggcagaca
  2101 agcacatggc aaggcggaca gcgcctcctg tgaaaatcgt tctacctcgc aaccggagtc
  2161 cgtgcacaag ccgcaggact cgggcaaggc cgagaagggc ggcggcaagg ccggggtggc
  2221 ggccatggac gagatccccg tcctcaggcc ctccgccaag gagttccacg atccgctcat
  2281 ctacatcgag tcggtccgcg ctcaggtgga gaagttcggg atgtgcaggg tgatcccccc
  2341 tccggactgg cggcccgagt gcaagctcaa cgatgagatg cggtttgtca cgcagattca
  2401 gcacatccac aagctgggcc ggcgctgggg ccccaacgtg cagcggctgg cctgcatcaa
  2461 gaagcacctc aaatctcagg gcatcaccat ggacgagctc ccgctcatag ggggctgtga
  2521 gctcgacctg gcctgctttt tccggctgat taatgagatg ggcggcatgc agcaagtgac
  2581 tgacctcaaa aaatggaaca aactagcaga catgctgcgc atccccagaa ctgcccagga
  2641 ccggctggcc aagctgcagg aggcctactg ccagtaccta ctctcctacg actccctgtc
  2701 cccagaggag caccggcggc tggagaagga ggtgctgatg gagaaggaga tcctggagaa
  2761 gcgcaagggg ccgctggaag gccacacaga gaacgaccac cacaagttcc accctctgcc
  2821 ccgcttcgag cccaagaatg ggctcatcca cggcgtggcc cccaggaacg gcttccgcag
  2881 caagctcaag gaggtgggcc aggcccagtt gaagactggc cggcggcgac tcttcgctca
  2941 ggaaaaagaa gtggtcaagg aagaggagga ggacaaaggc gtcctcaatg acttccacaa
  3001 gtgcatctat aagggaaggt ctgtttctct aacaactttt tatcgaacag cgaggaatat
  3061 catgagcatg tgtttcagca aggagcctgc cccagccgaa atcgagcaag agtactggag
  3121 gctagtggaa gagaaggact gccacgtggc agtgcactgc ggcaaggtgg acaccaacac
  3181 tcacggcagt ggattcccag taggaaaatc agaacccttt tcgaggcatg gatggaacct
  3241 caccgtcctc cccaataaca cagggtccat cctgcgtcac ctcggtgctg tgcctggagt
  3301 gactattccc tggctaaata ttggcatggt cttttctacc tcatgctggt ctcgagacca
  3361 aaatcacctt ccatacattg actacttaca cactggtgct gactgcattt ggtattgcat
  3421 tcctgctgag gaggagaaca agctggaaga tgtggtccac accctgctgc aagccaatgg
  3481 caccccaggg ctgcagatgc tggaaagcaa cgtcatgatc tccccggagg tgctgtgcaa
  3541 agaggggatc aaggtgcaca ggaccgtgca gcagagtggc cagtttgtcg tctgcttccc
  3601 gggatccttt gtgtccaaag tgtgctgtgg gtacagcgtg tctgaaaccg tgcactttgc
  3661 taccacccag tggacaagta tgggctttga gaccgccaag gaaatgaagc gtcgccatat
  3721 agctaagcca ttctccatgg agaagttact ctaccagatt gcacaagcag aagcaaaaaa
  3781 agaaaacggt cccactctca gtaccatctc agccctcctg gatgagctca gggatacaga
  3841 gctgcggcag cgcaggcagc tgttcgaggc tggcctccac tcctccgcac gctatggcag
  3901 ccacgatggc agcagcacgg tggcggacgg gaagaaaaag cctcgaaagt ggctgcagtt
  3961 ggagacgtca gagaggaggt gtcagatctg ccagcacctg tgctacctgt ccatggtggt
  4021 acaagagaac gaaaacgtcg tgttctgtct ggagtgtgct ctgcgccacg tggagaaaca
  4081 gaagtcctgc cgagggctga agttgatgta ccgctacgat gaggaacaga ttatcagtct
  4141 ggtcaatcag atctgcggca aagtgtctgg taaaaacggc agcattgaga actgtctcag
  4201 taaacccaca ccaaaaagag gtccccgcaa gagagcgaca gtggacgtgc ccccctcccg
  4261 tctgtcagcc tccagttcat ccaaaagtgc ttcgagctca tcatgaagat gccaacgccc
  4321 gtggtcgatt tatatatatt tttttgtaat tattatattc tagtttggag tacttgctgt
  4381 aggattcaag ctgtctttgc actagctcta aagaagattt tcttctggtt ttagagaact
  4441 aattttgttt tagcattaaa ctgttgaact tttttttgta cttagaaaac ctagatactg
  4501 cagtcagatt ttggaaactg ccgtatagtc actgttttaa aaaccccgga ggggctgtat
  4561 taatttgtat tgccccatgg ctgacaaaag cctttttttt tggttttgat tttttttttt
  4621 ttgtaactgt tggggggaaa aaggcttttt aacccatttt tgaagagggt gaagtttgga
  4681 gaacaaattt aaaaaccatc agtcatgtga gcagattttt tagaagggat aggagacaca
  4741 cgcgcacaca cacacacaca cgaaacttga aatggctttg ctttggctgt cgtcttctgc
  4801 cgtgtgccag atgagcttgt gatctgggaa gccggggcac ccccgttttg tttctctggg
  4861 cggttgtggc agctgaaggc ggacgttgtt tcctaaccat aggtggaacg aggagacggg
  4921 agcgagtggg ctctccacca gcacatcact atgcatctgt tccaggaaag aagaaaagcg
  4981 agcgaggaag acggaaaaga ctgcctgcct tggaggggtc acatgaggga gacctgtgcc
  5041 tgatttcatt aggaaatcca ttctgttatt ttttggtgct gttggctact ttatcaaaaa
  5101 acccttcaat agcatcctta agatttaaaa aaaaaaaaaa aaaaaaggaa aaaaaagtga
  5161 tggaagccgt aagtgcttct ttgtcatcga cgtgcaatct ttctaacatt ccatctccat
  5221 ctcaccgctt cttgtttgac accttcacaa gtcagcatta atctttcttt taaaacttgt
  5281 ttcatttatg atcatgtaga gagccactag gaggcctgca gttatttttg aatgtgaaaa
  5341 tgcatttgcg ttcatcttgt ctattttttc tcttcatgtt gtaacaaaaa ggaaaaaaga
  5401 aaaaaaaatc ccatcccttt tgtacatatg cctgtaaatt gttttaaata cttgagcctt
  5461 tttctcggtg gggggtgggg aggggggtga gaagacaaga tgaagaaaag ccttacattt
  5521 cagtttcttc atcggttgga ttggatgctt acagggtttt tcttgtaaca tttataagtg
  5581 ctgcttacat cactgaacaa caacaaaaaa ataataatgg agtagctgtt gcccttctcc
  5641 ggttgtgtgt acagtatgtg tggaataaaa aagggaaact gttttcacaa gctgttcttt
  5701 gtttcataat tggattcatc aatcccgtag ctacccatat tgcactgagc ttgccagtgg
  5761 tgactgccag gaacgtccta tgatccactt tgttggttgt tgttgcagaa gactgaactg
  5821 ttttggaata tttaacaatt acagaaacag tcaagtgttt tccaatgtgg ttgtccggtt
  5881 tctatggcct tgctgtgtac tttccctctt tttgacagta aacttctgcc tatggcttac
  5941 agtttgacat ttaatttatt agcgctgctc tgcacccctc ccttgggagg gagacttcat
  6001 gtggtttatt gcgagttttt tgtttacttt tcaggtttgt actacaaggt ttaataataa
  6061 aaacaaagtt ttttggacat ttgtctgtct tgtggaaaaa aaaaaaaaaa aa
YY1 (accession No. NM_003403):
(SEQ ID NO: 110)
        1 agggcgaacg ggcgagtggc agcgaggcgg ggcgggctga ggccagcgcg gaagtctcgc
      61 gaggccgggc ccgagcagag tgtggcggcg gcggcgagat ctgggctcgg gttgaggagt
    121 tggtatttgt gtggaaggag gcggaggcgc aggaggaagg gggaagcgga gcgccggccc
    181 ggagggcggg aggaggcgcg gccagggcgg gcggttgcgg cgaggcgagg cgaggcgggg
    241 agccgagacg agcagcggcc gagcgagcgc gggcgcgggc gcaccgaggc gagggaggcg
    301 gggaagcccc gccgccgccg cggcgcccgc cccttccccc gccgcccgcc ccctctcccc
    361 ccgcccgctc gccgccttcc tccctctgcc ttccttcccc acggccggcc gcctcctcgc
    421 ccgcccgccc gcagccgagg agccgaggcc gccgcggccg tggcggcgga gccctcagcc
    481 atggcctcgg gcgacaccct ctacatcgcc acggacggct cggagatgcc ggccgagatc
    541 gtggagctgc acgagatcga ggtggagacc atcccggtgg agaccatcga gaccacagtg
    601 gtgggcgagg aggaggagga ggacgacgac gacgaggacg gcggcggtgg cgaccacggc
    661 ggcgggggcg gccacgggca cgccggccac caccaccacc accatcacca ccaccaccac
    721 ccgcccatga tcgctctgca gccgctggtc accgacgacc cgacccaggt gcaccaccac
    781 caggaggtga tcctggtgca gacgcgcgag gaggtggtgg gcggcgacga ctcggacggg
    841 ctgcgcgccg aggacggctt cgaggatcag attctcatcc cggtgcccgc gccggccggc
    901 ggcgacgacg actacattga acaaacgctg gtcaccgtgg cggcggccgg caagagcggc
    961 ggcggcggct cgtcgtcgtc gggaggcggc cgcgtcaaga agggcggcgg caagaagagc
  1021 ggcaagaaga gttacctcag cggcggggcc ggcgcggcgg gcggcggcgg cgccgacccg
  1081 ggcaacaaga agtgggagca gaagcaggtg cagatcaaga ccctggaggg cgagttctcg
  1141 gtcaccatgt ggtcctcaga tgaaaaaaaa gatattgacc atgagacagt ggttgaagaa
  1201 cagatcattg gagagaactc acctcctgat tattcagaat atatgacagg aaagaaactt
  1261 cctcctggag gaatacctgg cattgacctc tcagatccca aacaactggc agaatttgct
  1321 agaatgaagc caagaaaaat taaagaagat gatgctccaa gaacaatagc ttgccctcat
  1381 aaaggctgca caaagatgtt cagggataac tcggccatga gaaaacatct gcacacccac
  1441 ggtcccagag tccacgtctg tgcagaatgt ggcaaagctt ttgttgagag ttcaaaacta
  1501 aaacgacacc aactggttca tactggagag aagccctttc agtgcacgtt cgaaggctgt
  1561 gggaaacgct tttcactgga cttcaatttg cgcacacatg tgcgaatcca taccggagac
  1621 aggccctatg tgtgcccctt cgatggttgt aataagaagt ttgctcagtc aactaacctg
  1681 aaatctcaca tcttaacaca tgctaaggcc aaaaacaacc agtgaaaaga agagagaaga
  1741 cccttctcga ccacgggaag catcttccag aagtgtgatt gggaataaat atgcctctcc
  1801 tttgtatatt atttctagga agaattttaa aaatgaatcc tacacaccta agggacatgt
  1861 tttgataaag tagtaaaaat taaaaaaaaa aaactttact aagatgacat tgctaagatg
  1921 ctctatcttg ctctgtaatc tcgtttcaaa aacacagtgt ttttgtaaag tgtggtccca
  1981 acaggaggac aattcatgaa cttcgcatca aaagacaatt ctttatacaa cagtgctaaa
  2041 aatgggactt cttttcacat tcttataaat atgaagctca cctgttgctt acaatttttt
  2101 taattttgta ttttccaagt gtgcatattg tacacttttt tggggatatg cttagtaatg
  2161 ctacgtgtga tttttctgga ggttgataac tttgcttgca gtagattttc tttaaaagaa
  2221 tgggcagtta catgcatact tcaaaagtat tttcctgtaa aaaaaaaaaa gttatatagg
  2281 ttttgtttgc tatcttaatt ttggttgtat tctttgatgt taacacattt tgtataattg
  2341 tatcgtatag ctgtattgaa tcatgtagta tcaaatatta gatgtgattt aatagtgtta
  2401 atcaatttaa acccatttta gtcacttttt ttttccaaaa aaatactgcc agatgctgat
  2461 gttcagtgta atttctttgc ctgttcagtt acagaaagtg gtgctcagtt gtagaatgta
  2521 ttgtaccttt taacacctga tgtgtacatc ccatgtaaca gaaagggcaa caataaaata
  2581 gcaatcctaa agcaagaata tggcagaaca agatctgtaa gcacagtctt attttctttt
  2641 gttgtccaga atacttataa ttcttgagcc tcccagaaat tggaagctaa ataaagcaac
  2701 tcaagtttcc tttattttgc actcaattac agtgattatt gatgaaagcg atgcatggat
  2761 attttaatac ttcctacatg tcctgacttc tgaaagagag taggtaacag gcatcccgag
  2821 ttcaggaact acctcagaac accccaggcc aggttggtca taggctgtga ttttagcccc
  2881 cggcaagtgt gagtgaagca tctgtaccac cgcgcaggct gagcgcctgc gcagggtaag
  2941 gtgccacctg gcagtggggc acacagaggg aagaccaggc ctgtccatca gccggctgcc
  3001 ttcagaggca gctccagcag gaccttggct tgtctgacag gaaatgcttg tggtcgttgg
  3061 ttatttggtt tgagagccct tgttcctcca tctagtggag tccttattaa atgctagcaa
  3121 tgtggcaatt gagtgccagt agcttaattt catgtttct
CBX2 (accession No. NM_005189):
(SEQ ID NO: 111)
        1 ggcggtccgg gcgggtgact ggcggcgggc gccgcggtcg ggctggctgc cgggcagcat
      61 ggaggagctg agcagcgtgg gcgagcaggt cttcgccgcc gagtgcatcc tgagcaagcg
    121 gctccgcaag ggcaagctgg agtacctggt caagtggcgc ggctggtcct ccaaacataa
    181 cagctgggag ccggaggaga acatcctgga cccgaggctg ctcctggcct tccagaagaa
    241 ggaacatgag aaggaggtgc agaaccggaa gagaggcaag aggccgagag gccggccaag
    301 gaagctcact gccatgtcct cctgcagccg gcgctccaag ctcaaggaac ccgatgctcc
    361 ctccaaatcc aagtccagca gttcctcctc ttcctccacg tcatcctcct cttcctcaga
    421 tgaagaggat gacagtgact tagatgctaa gaggggtccc cggggccgcg agacccaccc
    481 agtgccgcag aagaaggccc agatcctggt ggccaaaccc gagctgaagg atcccatccg
    541 gaagaagcgg ggacgaaagc ccctgccccc agagcaaaag gcaacccgaa gacccgtgag
    601 cctggccaag gtgctgaaga ccgcccggaa ggatctgggg gccccggcca gcaagctgcc
    661 ccctccactc agcgcccccg ttgcaggcct ggcagctctg aaggcccacg ccaaggaggc
    721 ctgtggcggc cccagtgcca tggccacccc agagaacctg gccagcctaa tgaagggcat
    781 ggccagtagc cccggccggg gtggcatcag ctggcagagc tccatcgtgc actacatgaa
    841 ccggatgacc cagagccagg cccaggctgc cagcaggttg gcgctgaagg cccaggccac
    901 caacaagtgc ggcctcgggc tggacctgaa ggtgaggacg cagaaagggg agctgggaat
    961 gagccctcca ggaagcaaaa tcccgaaggc ccccagcggt ggggctgtgg agcagaaagt
  1021 ggggaacaca gggggccccc cgcacaccca tggtgccagc agggtgcctg ctgggtgccc
  1081 aggcccccag ccagcaccca cccaggagct gagcctccag gtcttggact tgcagagtgt
  1141 caagaatggc atgcccgggg tgggtctcct tgcccgccac gccaccgcca ccaagggtgt
  1201 cccggccacc aacccagccc ctgggaaggg cactgggagt ggcctcattg gggccagcgg
  1261 ggccaccatg cccaccgaca caagcaaaag tgagaagctg gcttccagag cagtggcgcc
  1321 acccacccct gccagcaaga gggactgtgt caagggcagt gctaccccca gtgggcagga
  1381 gagccgcaca gcccccggag aagcccgcaa ggcggccaca ctgccagaga tgagcgcagg
  1441 tgaggagagt agcagctcgg actccgaccc cgactccgcc tcgccgccca gcactggaca
  1501 gaacccgtca gtgtccgttc agaccagcca ggactggaag cccacccgca gcctcatcga
  1561 gcacgtattt gtcaccgacg tcactgccaa cctcatcacc gtcacagtga aggagtctcc
  1621 caccagcgtg ggcttcttca acctgaggca ttactgaagc cccggcgcca ccagctgcgc
  1681 ggtcttactc cccttccctg cctatggtgt cgcttggcta agtgactccc agcccaagcc
  1741 ccctcaagag tctgggtcgg gggaggagga gtgggtggcc tccttgatgg gcaggcttgg
  1801 aagggacttc tcccgcaccc cactctgtcc caggacatag ggcagggggc ctcactgcct
  1861 tgttggtctc caccttgttc ctacctctgc aggcctcttt gctctcccct cttgcctcag
  1921 gaaacccggt ggcacctgtg gctccaggtg actgtcttga acagagcggg cttcttcatg
  1981 gctgcgttgt tgctgagttt gaactgctcc tccctggcct gcgtgactga atcacagctt
  2041 tggtccctgt cttgcagggg ctgaggtgtc aggaggggac ttctggccca ccttgccttc
  2101 agccctggag tgggcagaga gtattgtggg gaggcatggc cagtgggact agtgttccct
  2161 ccatctggcc acagcttttg ggagatgggg tgggcagggg tggtcctggc tggcattgcc
  2221 tgagccggca gtgatgaagt ggggagcttg cccttgacag gtgggggctg gctggggcct
  2281 taatgtgaaa agacagtggc aggcagctgg agtagagcga gcccagcagc cctaaaaggc
  2341 tgccttcatg gccatctagc cccagttcag ggcagcatcc atagcccaca agccagcgtg
  2401 ggtggggcgg gggtggtccc acagctgggt tccacctgaa gagcctccgt gcctcggagc
  2461 aggagaggca ggctatggct gccaccctcc ctcctgcctg tgtcccagtg agaactgacc
  2521 tgagtcccct tccaaaccca gacccacctc ctgccccagg cccactgaag catgttccat
  2581 ttctaaaaag cccagagttc agtgtgtccc aaggaaaacc caaagtggag gtgctcaggt
  2641 ccaggggagt ccagtgggca ggacccttgg caggcaagcc cctcccttca ctcccaggac
  2701 ctaccttctg ctagtaaagg actggcttca ttctaattat ggcccacaga ctgccccgga
  2761 gacctggagg acagcagtgc tcgcacttgg gtgtccatgg gcccgtctgc cggctctgcc
  2821 tgtgctgcaa gtgttggccg tgggtccagc caacaactcc ctacgtcctg tgtggggccc
  2881 tgcccaagtg gatgaggcat tccttgagga gtatcatttt ccctgacaat ccccatcacc
  2941 tttaggggtt ccctgcttgg ctcctttcca gctgaaaaac tagacctgtg ccattgggga
  3001 agctggacaa agtctagggg gcccgcctgg tagagggtcc cgggaagctg gatctgtcag
  3061 cctcggccct gaggcccctg ttaactcaag actgtgagct gcctctaggt ggtcacgtct
  3121 gggagctagc ttgtatggct tctgaccagt atcaggattt ctgttctgag agcagcgtgg
  3181 gcagcaaggc agggcagccc agaggtggca gcggcaggca atctggtcac taggtctttg
  3241 tgatgccaaa aataaaagag ggtggggtgg gtgctttctg ttcctctgat tggatggagt
  3301 ccgccagcag gcatggggct acattccagt gcctgactat agggaggcac tcctgattcc
  3361 atggagcagc ccggactttg agaatgggct ctggtttgcg gggggcaggc gtaccagact
  3421 gcaagacccc ccagtacctc accgtgccaa ataggaagag gtggccttgg tgtagccaaa
  3481 tggatctttt taacagtgtg cctttgggga gggacccatg tccatggctt cgttgagggc
  3541 catccatatg ccagctgggg gccagcccac agtggccata ttggctgcag caggaatggt
  3601 gcccacctcg gcgaattgaa gggctaagag tcccagatag ctaggccaga gctggaagca
  3661 gacagtaagg ggaagagctg ctcccacagg agagggagag attccagctc actgcgcagc
  3721 ctgggaggag gcgtggatcc tggcacgctg agcctcaggc accagcctcc ctgtgctcga
  3781 cagcaaagtc ttgactcctt cctgctgagc actgtgctac cttcactgct ccaaagccag
  3841 actaacagct ctccaagccc ttggggtgac tcggcttcca ggagctgttg gagaaatgag
  3901 gatgtctgtc cctgtctgcc tgggcaggcc agattcctcc ccagcagccg ggtctctcca
  3961 gaccctgatt cggtgccttt ctgtttacca gctacttcaa tcccaaagtt tgaatctgca
  4021 gataccttac tcccagccac tttgccttct tactgtgttg tgtgtttttc ctggtgcttc
  4081 aagagcgtgt gcagggcaag tgccgtcact gggaactgca ccagatgctc agacttggtt
  4141 gtcttatgtt taccaataaa taaaagtaga ctttttctat ttttatttgc tgctatttgt
  4201 gtgtgtgttt gtgtttgtgt agctaggtat ctggcacttc tgacgatgca ttgttgcttt
  4261 tttcc
CBX4 (accession No. NM_003655):
(SEQ ID NO: 112)
        1 agccggggcg ggcgcgggca gcggcgggcc ggccgggctg tgcggggcga gcggcggcgg
      61 cggcgggggc gcttcggccg gggcggcagc tgggcgccgg cgggagctag cagcgtctgc
    121 agccgcgccg gccgccagcg ccccggcgcg ctccggctcg gccatggagc tgccagctgt
    181 tggcgagcac gtcttcgcgg tggagagcat cgagaagaag cggatccgca agggcagagt
    241 ggagtatctg gtgaaatgga gaggctggtc gcccaaatat aacacgtggg aaccggagga
    301 gaacatcctg gaccccaggc tgctgatcgc cttccagaac agggaacggc aggagcagct
    361 gatgggatat cggaagagag ggccgaagcc caaaccgcta gtggtgcagg tgcctacctt
    421 tgcccgtcgt tccaatgtcc tgaccggcct ccaggactcc tccactgaca accgtgccaa
    481 gctggatttg ggcgcgcagg ggaagggcca ggggcatcag tacgagctca acagcaagaa
    541 gcaccaccag taccagccgc acagcaagga gcgggcgggc aagcccccgc cgccgggcaa
    601 gagcggcaag tactactacc agctcaacag caagaagcac cacccctacc agcccgaccc
    661 caaaatgtac gacctgcagt accagggcgg ccacaaggag gcgcccagcc ccacctgccc
    721 ggacctgggg gccaagagcc acccgcccga caagtgggcg caaggtgcgg gggccaaagg
    781 ctacctgggg gcggtgaagc ccctggccgg tgcggcgggt gctccaggca aaggctccga
    841 gaagggcccc cccaacggaa tgatgccggc ccccaaagag gctgtgacgg gcaacgggat
    901 tgggggcaag atgaagatag tcaagaacaa gaacaagaac ggacgcatcg tgatcgtgat
    961 gagcaaatac atggagaacg gcatgcaggc ggtgaagatc aagtccggcg aggtggcaga
  1021 gggggaggct cgctccccca gccacaagaa gcgggcagcc gacgagcgcc accctcctgc
  1081 cgacaggact tttaaaaagg cggcgggcgc agaggagaag aaggtggagg cgccgcccaa
  1141 gaggagggag gaggaggtgt ccggggttag cgatccgcag ccccaggatg ccggctcccg
  1201 caagctgtcc ccgaccaagg aggcctttgg agagcagccc ctgcagctca ccaccaagcc
  1261 cgacctgctt gcctgggacc cggcccggaa cacgcacccg ccctcacacc acccgcaccc
  1321 gcacccccat caccaccacc accaccacca ccaccaccac cacgccgtcg gcctgaatct
  1381 ctcccacgtg cgcaagcgct gcctctccga gacccacggc gagcgcgagc cctgcaagaa
  1441 gcggctgact gcgcgcagca tcagcacccc cacctgcctg gggggcagcc cagccgctga
  1501 gcgcccggcc gacctgccac cagccgccgc cctcccgcag cccgaggtca tcctgctaga
  1561 ctcagacctg gatgaaccca tagacttgcg ctgcgtcaag acgcgcagcg aggccgggga
  1621 gccgcccagc tccctccagg tgaagcccga gacaccggcg tcggcggcgg tggcggtggc
  1681 ggcggcagcg gcacccacca cgacggcgga gaagcctcca gccgaggccc aggacgaacc
  1741 tgcagagtcg ctgagcgagt tcaagccctt ctttgggaat ataattatca ccgacgtcac
  1801 cgcgaactgc ctcaccgtta ctttcaagga gtacgtgacg gtgtagccgg agggcgtcgg
  1861 aaggggaagc gccattcccg cgggggggcg gggagctgag cacctggggc ctcggggcgg
  1921 gctcccctct cgccaacccg ccaaccgcga gagacccagg ctggccccca gggtgaggac
  1981 gcccggagcg gaggtaacca tgttccccct gcggcggctg tcagacctgg gcggaggccc
  2041 cttccacgcg gtgccggcgg ggctcgccct ctcctgccct tccccgctgg agatggaccc
  2101 ccggaacgga cagggcagct ctgcgcccgg cctcagagtt ctagtattat attttaaccg
  2161 tgctaacttg tcaagtgctg actctactcc cgtttgtacg tggtgttatt attgaaatgt
  2221 attgtttgag ctcaaaaggc ccgaccaccc cccttcgggc tgctatatat atatttattt
  2281 gtaggtattt atatattgaa atataaaaac ctagatttat ggagtttcct ctagatcatg
  2341 ttatattcta tatcagacaa actattttct tttgaccttt cttcccctcc atccagtatt
  2401 tcggttgatt tcattttctc ccctctcttc cccttccacg aactgcaata ccagtaacct
  2461 tggtatatat tttttgatac tgtacacatg gatgtcttgt ttctatgtgc aaaaaaaaaa
  2521 aaaaaaaaaa gtttgttaaa aggctacacg agctctctag aaactgctgc tactagaaat
  2581 gtctaaacta taagcttcca actattacct gcttgaatgt aaatattaaa tggagatgtt
  2641 gaaggtgcaa aaaaa
CBX6 (accession No. NM_014292):
(SEQ ID NO: 113)
        1 gtgacggccc gcagctggaa cgcgagcgcg cgccccgccg cgctcccgcc cgccggggcc
      61 tgggcgctgc ggcgcgtgcg cgagcggtgc cgcaccggcc gcgggcgcag ggagtattat
    121 gggctgtggg tgccgctgag caagatggag ctgtctgcag tgggcgagcg ggtcttcgcg
    181 gccgaatcca tcatcaaacg gcggatccga aagggacgca tcgagtacct ggtgaaatgg
    241 aaggggtggg cgatcaagta cagcacttgg gagcccgagg agaacatcct ggactcgcgg
    301 ctcattgcag ccttcgaaca aaaggagagg gagcgtgagc tgtatgggcc caagaagagg
    361 ggacccaaac ccaaaacttt cctcctgaag gcgcgggccc aggccgaggc cctccgcatc
    421 agtgatgtgc atttctctgt caagccgagc gccagtgcct cctcgcccaa gctgcactcc
    481 agcgcagccg tgcaccggct caagaaggac atccgccgct gccaccgtat gtcccgccgt
    541 cccctgcccc gcccggaccc gcaggggggc agccccggac tgcgcccgcc catttcgccc
    601 ttctcggaga cggtgcgcat catcaaccgc aaggtgaagc cgcgggagcc caagcggaac
    661 cgcatcatcc tgaacctgaa ggtgatcgac aagggcgctg gcggcggggg cgccgggcag
    721 ggggccgggg cgctggcccg ccccaaagtc ccctcgcgga accgcgttat aggcaagagc
    781 aagaagttca gcgagagcgt cctgcgtaca cagatccgcc acatgaagtt cggcgccttt
    841 gcgctgtaca agcctccgcc cgcccccctg gtagccccgt cccccggcaa ggctgaggcc
    901 tcagccccgg gccctgggct acttctggcc gcccccgccg ccccctacga cgcccgcagc
    961 tctggctcct ccggctgccc ctcgcctaca ccacagtcct ctgaccccga cgacacgccc
  1021 cccaagctcc tccccgagac cgtgagccca tccgccccca gctggcgcga gccggaggtg
  1081 ctcgacctgt ccctccctcc cgagtcggca gccaccagca agcgggcacc gcctgaggtc
  1141 acagctgctg ccggcccggc acctcccacg gcccctgagc ccgccggtgc ctcctccgag
  1201 cccgaggctg gggactggcg ccccgagatg tcaccctgct ccaatgtggt cgtcaccgat
  1261 gtcaccagca acctcctgac ggtcacaatc aaggaattct gcaaccctga ggatttcgag
  1321 aaggtggctg ctggggtagc aggcgccgct gggggcggtg gcagcattgg ggcgagcaag
  1381 tgagggggct ccaccaagga ggggggcttg ggggggccct cctgcccgaa gtcatactct
  1441 tgctcccacc ccacccttgc ccccagccct ctctccctgt gctttgcttg tctcaaatgg
  1501 ctcggtgttg acccagggat ggggctgggt agttggggtc ccagaaagcc gggggtaggg
  1561 gccaccctgg aatggggcag gggaagggca caccccctgc ccatgcatgg tagcccactg
  1621 ggtggtttct ggaaagccct agaaactagg gttcctctgc cccttccaca tcccacctgt
  1681 ctctctagct tgcttcctgc tctcctgtgc ggcgtctgat ttctcggtgc taacctggca
  1741 gctgtggggc ccttaggagc cccccaccga gggtggacac agtccctttc cttcctgcag
  1801 atgcctaggc aggaggaggg cttcctgcct gtttggcaaa gtcccaggca gaggccaagg
  1861 atgaggcctg actcggctcc tccctccaca tcagccaggg catcagaagt tgggccaggg
  1921 cggggtcttc cctgctcgat tttggacgag gcctaagtag accccctatg ccctgcccca
  1981 gccctggctc tttcctaacc ccctcaacgg tgggaggaac tggcagaggg tgcgcctggc
  2041 cacagcctcc ccgcatctaa aggccccttc agttcttgac caaaggtgct acgagaacct
  2101 gccgtggaaa cttccagttg tgcgtctgcc ccactcgctg tgtttgtccg tgggttcata
  2161 catgcattgg gtgctaggcc ccaggctgcc gggtggcacc ctttacagtt cctttgaaca
  2221 ggggcattga aggcctggac tgcctctcgc ctcagtaggc ctggggacca ggcttgggtc
  2281 tggaggtttg ctgtggaagt caccaggcct cccctcctgg cccaggtgtg ctgggggcac
  2341 cgtgcccccc acccccctgc cctcctcagg gtggtcagcc caacctgtcg gaccttcact
  2401 tcacatcatg gtggggaccg agatagagag ggagacccca ttccaagctc cctcttcctc
  2461 cgggtgtttg gggaggatgc tgaagaatcc attcccgagg gcctcccggc ttgtcccagc
  2521 ccctcttttg cttctgacca cggaggcttt ctcacagccc agcctgcctg aagcaaagga
  2581 ggctcccgtg tcctgggcag cttctgtttc cctctgctgc ctgggagctg aggcacccgt
  2641 gccagtggca gaggccacag ccccagcctt aggccaggcc ctgggagggc aggcaggcaa
  2701 aggggagacc agagggtctg tgttctccag gagaatgagg gtgttggtcc cagaattggg
  2761 accggggccc cgctggccag ccctgggcca cttcccgggt ctccattgtg cgtgggtggc
  2821 gtgttccagg cgtggctgga gctggcttcc tggctgtgct gccatgggcc cctccctcag
  2881 aagcacgttg gcaggaggcc gatcagaacc ctagcgcctt tggtcctaag aatgggaggc
  2941 tgccttcctt cccaatctcc ctgccagggc ccacagcgtg gccctagccc tcccctcccc
  3001 gggatgtaga acggggaccc tcgcagggtt ggggcggggg ctgatactcc tcggcccctc
  3061 cctaccctgc cctgtgtgtt ggctttgtgg ccgtccaagt gccaattggc ttttcgccca
  3121 aataagggct ggtatttctc ctctgtcctt ggaggtgatt tccccctgac cccctccccc
  3181 aggtgagtga ccacctgggt gccagttaca ggtgtttcca gagaccatag aaatgtgttt
  3241 tcctgagagt tcgtgtcatt cgtgactttt ttgtaaagaa gttgtgtttt cagaggtgat
  3301 tttatgacag gaaagtgaaa gaattagttt tgcaaaaaaa caaaaacaaa aaaagaggaa
  3361 aaaaaaaaga aatagaaaaa aatattgtgg gattcctatg ggggggtggc gggggagaaa
  3421 gagctattta agaaaaaata gtaacgcagt gattgcacag gtgaggtggc aatgtcagga
  3481 tggggcggag gcctgggccc agctggcagg tcccctggca tcgcaggcac tgtggagagg
  3541 gcctggaccc agatctccac acccgtgctt gctcaaaggg aaggacaaca gcgggccccc
  3601 gggagctaac ccaagctgca ggtcccggca agctgaggtt tgggagggtg ggggttgtca
  3661 ctggtgattt tctccagggg gctggtgagt gggcagtttg gtttcttgcc cccttctgtt
  3721 cctttcccag ttgttgggcc atctggtccc caccaccgcc accctatggg ggagacctcc
  3781 ctccccacgg gtcaccctaa agcccacaac ctctctgagc ctccctggcc tgaaagggga
  3841 tgcaggcttc aggaggcaag aagctgggcc cctgggggtg gctggggaga gggaatgcat
  3901 ttcccttgcc acaggtggtc tgcttctgct ggcctgagct ccaagtggag cagcccgggc
  3961 cagccttggt gcatgaagag gcaccaggca caccgccttg aggtgggcag tgcccatggg
  4021 ggcccgagtg gatgggaccg agggtgagtg gagcctcctt cctcccctct ctagtacccc
  4081 cgcctcccac acacttgcac ggatcggcct cccttgggag atcagcctcc atgggcccct
  4141 cgtccaccct tgctgctttc catttgccta attaccaagc agaagttgca atctggtttg
  4201 ctttattttt gtatgtgaaa taacccccaa agcccaatct cctcctacgt tcaatattgg
  4261 ttggggcatc cgtcatctcc ccttaagtgc gccccctccc cacccaagta tcataggaaa
  4321 ccggtgaggt ctggtgtctc tggtttgaga cggtaagttg ggacccatcc ctgtctgggt
  4381 gcccactctg acctttagtt tgcccttctg tgaaatgggt gtattgggta gcaagccctc
  4441 ttcagaaagc gctgctggtg tcagagcagc tgcccagtac caggtggggg gtcaaggttg
  4501 ctggtactgg ggcccccagc tgcccacaac ccctctttgt tctcaccctg caaaggggtc
  4561 aaggtcaaaa tgagcctcat ccttcctatg atctgggaag aggtgatgat caagtcccca
  4621 acttcagtgt gaggtggaca gagttggggg gatggcccct ttttgaagag gtgaaaatgg
  4681 ttttggagaa gcgcagctgc ttcactgggg gaatgcggca gggactgggg cccaggatgc
  4741 tttggcctat ggggaaaagc cctttaaaag gcagggccca ggccctggag ccagcacaag
  4801 actggcctcg agcccctgag ccaggaggtc ctggaggaga gccaggccgg tgggcccgcc
  4861 caaggctgga gggtcagccc caacagggag ctgggttggc cagggggctg gactgctacc
  4921 agcctctctg gcctatgggg acccaagagg acacatcccc cttttgccca ctcttctgtg
  4981 tcattttgtt gttttggttt gtggtggttt ttcttttttc ttttgttttt cttttttctt
  5041 tctttctttt tttttttttt tttttttttt tttgcacttc gcccacacag gacagtggag
  5101 ccccacctgg tcagttccac ttccgggctc ccatgcactt gcccaaggcg gcctctttgg
  5161 gacggggatg gtttgaggaa acacttttaa agaaaaaagg aagacattga aaggttttag
  5221 tttcttccct atctgcatgt cctctcatat agaaagccca gaattagggg ctagaactcc
  5281 aggagagggt ctccccgact catctcttgc tgacggtcac caggatgcag aaatagggag
  5341 atggttagtg ggggccaaag atgccccctc ccaggccttc gtggttccct cctccgcccc
  5401 ctgcaatctt tggaggagtc agtgcctcac tccagcagtg agtgcctact gtatgcaggt
  5461 agtcagccag gcaaagagag actaacggtc tcatggggga accctcttgc gggaggccgg
  5521 gtagctggag cgaagcgttc cggctgccct tgctgctggg tggagtggag agggagactt
  5581 ctttttgttg gttttaattt aaaaacacaa aggcctaaag aaatacgtat cttataattt
  5641 ttttaatttt tgagacgttc atttaatgaa ttgtgcacga atgaattcta tatatataaa
  5701 atatacatat atagctctat atttggggag gggcactgtc tcttttttct ctcattttta
  5761 aaatgaagtg ttgttgcctt tgtatgtggt tcaaccatcc agctcccagc tggctaaact
  5821 ttgcctccag tggtcaaaga tgggaaaaga gtggggttgg caggagatgg aaaacggagg
  5881 tgccgcccca gcatgggggg caggtccccc agtccaccct gcccctcccc ctgtggagaa
  5941 gacgcttagt tgggggtgtg ggtttgggct ccattctgga ttcggcggtt ccgggggagg
  6001 ggtgggtctg tgccgattac tctgtcttgt acgtttgttc tgctgctctt caatattgta
  6061 tcaacgccag gaaagggggg tgaaaagcct cttttacccc ccaaataaat tgtcacattc
  6121 cgaagctgag gcctagcccc taggttgggg tgtgtctgtg tcttc
CBX7 (accession No. NM_175709):
(SEQ ID NO: 114)
        1 ccagccccag catcgcgcgc cgcagccgcg gccccgcagc tccgcccccg gcccggcccg
      61 gccccgggcc cgctcgcccg ccgccccgca tggagctgtc agccatcggc gagcaggtgt
    121 tcgccgtgga gagcatccgg aagaagcgcg tgcggaaggg taaagtcgag tatctggtga
    181 agtggaaagg atggccccca aagtacagca cgtgggagcc agaagagcac atcttggacc
    241 cccgcctcgt catggcctac gaggagaagg aggagagaga ccgagcatcg gggtatagga
    301 agagaggtcc gaaacccaag cggcttctgc tgcagcggct gtacagcatg gacctgcgga
    361 gctcccacaa ggccaagggc aaggagaagc tctgcttctc cctgacgtgc ccactcggca
    421 gcgggagccc tgagggggtg gtcaaggcgg gggcacctga gctggtggac aagggcccct
    481 tggtgcccac cctgcccttc ccgctccgca agccccgaaa ggcccacaag tacctgcggc
    541 tctcgcgcaa gaagttcccg ccccgcgggc ccaacctgga gagccacagc catcgacggg
    601 agctcttcct gcaggagcca ccggccccag acgtcctgca ggcggctggc gagtgggagc
    661 ctgctgcgca gccccctgaa gaggaggcag atgccgacct ggccgagggg ccccctccct
    721 ggacacctgc gctcccctca agtgaggtga ccgtgaccga catcaccgcc aactccatca
    781 ccgtcacctt ccgcgaggcc caggcagctg agggcttctt ccgagaccgc agtgggaagt
    841 tctgaatcac cgtttttact cttcttaaac tgttttcttt tgggcttggg gtgggacttc
    901 cagagatagg gatgggttgg gggcggggta attattttat ttaaaaaaat accgagcagc
    961 aaaaggggag aagatcccac tactctccca ccacctgccc tttctctgag ggacgtttac
  1021 cacgaggcct caggctgggg atggagagag ttgctctggg agttggggta ccacccccag
  1081 ggcaggatgg ggacaggatc acctgcccgg gacaccacca ttatcattct cctctagtga
  1141 cgcagcagct ggttctggga gttaaaggag cattggaagg cccaaaccct ctcccttgag
  1201 tggccacccc agcctggttg gctggttttc cccttttctc ttgtttcaat tgggtcttta
  1261 ccttgaactc tcctctctgg ctttgcggtg ggctgtggag gctggttttg accaaaagtg
  1321 agtggggcgg gaggaagggg caggaggaag ggttgaggtt acttggggcg agtcccttcc
  1381 ccttcagaga ggcttctatc cttcccaggg aggaggcgcc gctgagaccc ttctgctgag
  1441 agctctgccc tcccctcatc acctggcctg tgcagaaacg ctcatgcaca cctggctgca
  1501 caggtgtgca cgcattaccc ttcgcgtgta cgttcccatg tgccccgtga aagcatgtgt
  1561 ggctgcagac gtgtccacat gggccttgcg aacctgggtt agaaaccctg gccaggcgaa
  1621 cgtggggtga ttcacagcac aaaagacctc accaccacac ctgcactcac cccaccttgc
  1681 atgcaccttg ctacctgctt gcggctttca gtggagggca ggggtctggc acaggtgcga
  1741 tggcacccca tgctccaggc atacagatgt ggtttctcgg ctgcaccggg ccaggctgcg
  1801 ggtgtgcagg cgtctgctaa gttgtgtgat gtatcagcac aggctttgag acgtctggac
  1861 cctgtccttc ctcccgtgag gggttcttgt tctttctgac tcaggtgact tttcagccct
  1921 tccaattccc ctctttttct gccctcccct ccaactcagc caacccaggt gtgggcagtc
  1981 agggagggag ggagtgtccc accacgttct cagggcagcc cttgactcct aagccccttc
  2041 ctccttccat tctgcatccc ctccccatcc aacctaaatg ccacagctgg ggctgagctg
  2101 tattcctgtg gagggacctc tgccgtgcct ctctgaggtc aggctgtgct gtgtgatggg
  2161 caggctttgc cccagcccac ccctggcaag gtgcacttgt tttctggttt gtacaaggtg
  2221 tcctgggggc ccgtggcttc cctgccagtg aggagtgact tctccctctc ttccagtcct
  2281 gtaggggaga caaaaccaga ttggggggcc caaggggagc atggaaaagg ccggctcccc
  2341 tgtctttcct tggctgtcag agtcagggta acacacacca agagtggagt gcggccagca
  2401 agtttgagac ctgcccgccc tcctcgcagc tctgctctgt gtcctcagga agtcacagag
  2461 tctactgagg caaggagagg gtgattcttt ccccaaatcc cttcttccct ggttcccaaa
  2521 ccaaagacag cctgcagccc tttctgcatg gggtgctctg ttgacaggct tcccagatcc
  2581 ctgagtctct ctttccttcc tcctcgatct ttagttgtcc acggtcaatt cagtgcttcc
  2641 attgggggac agtcccctcc gggatgacct gattcacctc cagcccaggg aatggaatct
  2701 agaggaatac gtggggtggg tctggacaag gagcggcagg aatcaccacc catctccagc
  2761 tgtggagccc tgtggagggg aaggggaagc ttggggttca gaggggactc ttccaggaga
  2821 ggggtgccca gcggaggtaa agatgataga gggttgtggg gggtctctag ttgaatgttt
  2881 tggcccatga ctttggaaca tggctggcag cttccagcag aagtcacgct ccccatcccc
  2941 caggggacat aggacctttt tcctgcttcc tggtcacttt caaagaacta tttgcgcaat
  3001 ctgtgggtct gtggattcac ggggctttct gtgtgggtgc tgcagttgct tttgtctgca
  3061 gcagcaggac acatctttcc tcttactcag ccctttatgg cccatgggga actccgtggc
  3121 tcagggagag ctgaactcca ggggtgtgac ctgggacggg tgggcctgag gtgcccagct
  3181 cagggcagcc aggtggctca tgggctgtag tgagccagct ccctggggga aaaggctgtg
  3241 ggccgttagg accatcctcc aggacaggtg acctctatga ggtcacctac ggctgtggcc
  3301 gtgcaggcct ccttccagcc cagagtggcc cagtagagca aggcagacag tgacctccac
  3361 ccccgcagcc ctcttaaaag gccagtactc ttgggggtgg ggggagggtt tagaaagcat
  3421 ttgcccatct gcctttcttt cccccagccc ccacccgctt tgaatgtaga gacccgtggg
  3481 cacttttcct tttgtggtgg ggggtgcgga ggaggtaccc ccacccctgg cacagccgcc
  3541 tggaatgcag gactgtcact gctgttcggg tgatgacctc gttgccaagc tcctcctgtc
  3601 cccttgttct gggggcaggc gctgtgcttc tgtgaggtgg tttagctttt gctttcgaag
  3661 tggccagctg cggccaccag gtctcagcac aagagcgctt cctttgcaca gaatgagctt
  3721 cgagctttgt tcagactaaa tgaatgtatc tgggaggggt cgggggcacg agttgattcc
  3781 aagcacatgc ctttgctgag tgtgtgtgtg ctgggagagt cagagtggat gtagagcgcg
  3841 gttttatttt tgtactgaca ttggtaagag actgtatagc atctatttat ttagatgatt
  3901 tatctggtaa atgaggcaaa aaaattatta aaaatacatt aaagatgatt taaaaaaaag
  3961 aaaa
PHC1 (accession No. NM_004426):
(SEQ ID NO: 115)
        1 cccgccgcgg aggccgagcg agcccccagc ccagcctggc gactggggac cccggcacat
      61 gaggtggacg cccccgggga agacttgggt gcacagccag gcgagaaggt cttgagtcag
    121 acagagcacc agccttgggg accctggacc actatcatgg agactgagag cgagcagaac
    181 tccaattcca ccaatgggag ttctagctca gggggcagct ctcggcccca gatagctcaa
    241 atgtcacttt atgaacgaca agcagtgcag gctctgcaag cactgcagcg gcagcccaat
    301 gcagctcagt atttccacca gttcatgctc cagcagcagc tcagtaatgc ccagctgcat
    361 agcctggctg ccgtccagca ggccacaatt gctgccagtc ggcaggccag ctccccaaac
    421 accagcacta cacagcagca gactaccacc acccaggcct cgatcaatct ggccaccaca
    481 tcggccgccc agctcatcag ccgatcccag agtgtgagct ctcccagtgc taccaccttg
    541 acccaatctg tgctactggg gaacaccacc tccccacccc tcaaccagtc tcaggcccag
    601 atgtatctac ggccacagct gggaaaccta ttgcaggtaa accgaaccct gggtcggaat
    661 gtgcctctag cctcccaact catcctgatg cctaatgggg cggtggctgc agtccagcag
    721 gaggtgccat ctgctcagtc tcctggagtt catgcagatg cagatcaggt tcagaacttg
    781 gcagtaagga atcaacaggc ctcagctcaa ggacctcaga tgcaaggctc cactcagaag
    841 gccattcctc caggagcctc ccctgtctct agcctctccc aggcctctag ccaggcccta
    901 gcggtggcac aggcttcctc tggggccaca aaccagtccc tcaaccttag tcaagctggt
    961 ggaggcagtg ggaatagcat cccagggtcc atgggtccag gtggaggtgg gcaggcacat
  1021 ggtggtttgg gtcagttgcc ttcctcagga atgggtggtg ggagctgtcc cagaaagggt
  1081 acaggagtgg tgcagccctt gcctgcagcc caaacagtga ctgtgagcca gggcagccag
  1141 acagaggcag aaagtgcagc agccaagaag gcagaagcag atgggagtgg ccagcagaat
  1201 gtgggcatga acctgacacg gacagccaca cctgcgccca gccagacact tattagctca
  1261 gccacctaca cacagatcca gccccattca ctgattcagc aacagcaaca gatccacctc
  1321 cagcagaaac aggtggtgat ccagcagcag attgccatcc accaccagca gcagttccag
  1381 caccggcagt cccagctcct tcacacagct acacacctcc agttggcgca gcagcagcag
  1441 cagcaacaac agcaacagca gcaacagcag cagccgcaag ccaccaccct cactgcccct
  1501 cagccaccac aggtcccacc tactcagcag gtcccacctt cccagtccca gcagcaagcc
  1561 caaaccctgg tcgttcagcc catgcttcag tcttcaccct tgtctcttcc acctgatgca
  1621 gcccctaagc caccaattcc catccaatcc aaaccacctg tagcacctat caagccgcct
  1681 cagttagggg ccgctaagat gtcagctgcc cagcaaccac caccccatat ccctgtgcaa
  1741 gttgtaggca ctcgacagcc aggtacagcc caggcacagg ctttggggtt ggcacagctg
  1801 gcagctgctg tacctacttc ccgggggatg ccaggtacag tgcagtctgg tcaggcccat
  1861 ttggcctcct cgccaccttc atcccaggct cctggtgcac tgcaggagtg ccctcccaca
  1921 ttggcccctg ggatgaccct tgctcctgtg caggggacag cacatgtggt aaagggtggg
  1981 gctaccacct cctcacctgt tgtagcccag gtccctgctg ccttctatat gcagtctgtg
  2041 cacttgccgg gtaaacccca gacattggct gtcaaacgca aggctgactc tgaggaggag
  2101 agagatgatg tctccacatt gggttcaatg cttcctgcca aggcatctcc agtagcagaa
  2161 agcccaaaag tcatggacga gaagagcagt cttggagaaa aagctgaatc agtggctaat
  2221 gtgaatgcta atactccaag cagtgaacta gtagccttga cccccgcccc ttcagtaccg
  2281 cctcctacac tagccatggt gtctagacaa atgggtgact caaaaccccc acaggccatc
  2341 gtgaagcccc agattctcac ccacatcatt gaaggctttg ttatccagga aggagcagaa
  2401 cctttcccgg tgggttgttc tcagttactg aaggagtctg agaagccact acagactggc
  2461 cttccgacag ggctgactga gaatcagtca ggtggccctt tgggagtgga cagcccatct
  2521 gctgagttag ataagaaggc gaatctcctg aagtgcgagt actgtgggaa gtacgccccc
  2581 gcagagcagt ttcgtggctc taagaggttc tgctccatga cttgcgctaa gaggtacaat
  2641 gtgagctgta gccatcagtt ccggctgaag aggaaaaaaa tgaaagagtt tcaagaagcc
  2701 aactatgctc gcgttcgcag gcgtggaccc cgccgcagct cctctgacat tgcccgtgcc
  2761 aagattcagg gcaagtgcca ccggggtcaa gaagactcta gccggggttc agataattcc
  2821 agttatgatg aagcactctc tccaacatct cctgggcctt tatcagtaag agctgggcat
  2881 ggagaacgtg acctggggaa tcccaataca gctccaccta caccggaatt acatggcatc
  2941 aaccctgtgt tcctgtccag taatcccagc cgttggagtg tagaggaggt gtacgagttt
  3001 attgcttctc tccaaggctg ccaagagatt gcagaggaat ttcgctcaca ggagattgat
  3061 ggacaggccc ttttattact taaagaagaa catcttatga gtgccatgaa catcaagctg
  3121 ggccctgccc tcaagatctg cgccaagata aatgtcctca aggagaccta aggtggccct
  3181 cttgcacaaa ccagcctaag gcagacactc tccactgtcc aggttataac ctggtaccag
  3241 cagactttgc agggaagaaa gagttgttcc aatcatgtaa ccttctgtag gggattactg
  3301 agacagggaa gagaagtgca agaattggtt gctggtgcta catggcggca gctttgacat
  3361 tttctctggg ttctacttta ttttttaaaa tctttacagt tctcaccatt tcacgtacct
  3421 taatccaatc tttataaaag aggcagtcta gagaactagg actgctcagc cttatcctgg
  3481 agtggagcat ttagcccagg tcttaattct ccaagaggag gaatacatag tatggtaagg
  3541 caaggaactg ggtggaatgt caggttgcct gcccaatggg agaggtaggg tttttctagc
  3601 ttgtgtgaca gaagtagcaa aatctggtcc tcccccctcc cagtgtagct gtggctcaga
  3661 gttttttctt tttgttgtca cttactccct tgtgattgaa ttttttctcc tgcatccatg
  3721 gcaggatccc cagccagtat agagacttgg ttggcatctt ctgctgcagg gactaaaagt
  3781 atttgactgg ggcacatgtg gctgttgtca ttctttctgc atcccactgt tcccctccaa
  3841 tttatgttat tttctaccct gtttttcagt tccatctctg ctctgtccta tagctttata
  3901 aaaccagagt gtgtggggct gaggtcagga gtataagtac ctgccttagg cactattcct
  3961 tatataacaa aaatattaaa tatttttttc ctcagtaaaa ggatgaaaat tggtttcagt
  4021 tgtcttactc tattccagtc tttgcccact ttcacacaaa tgacaaggcc aatatgtttt
  4081 gtttgttttt taatcattaa gagtttttgt acaaaaggtg atggtttttt ttcttcattt
  4141 taaaacacca gggtgtgggg gagggatgca aacaaataac aaaaaagatg cttttgtaac
  4201 attattttcc ctgtttagaa agaaaaaaat cactccaata gtattgaaaa gtccaaagat
  4261 gaaatagttt cattttcttt tcctaaggct tataaaaggc cccctgcctg ttgattccat
  4321 ccctcttttg tgtccagtgg agccatgtta ctcttcagtg gcccaggggt tcactattaa
  4381 agaaagatca gtccaggttt ctgggcacat ggcctaaaca ggaagatgga agcatcagag
  4441 gattaaaaac ctttccccac agaaatgtgg gcaagaagac acttccctga gccagcagaa
  4501 gggacaggtg cagcagcatt ccacacccag cgcagaggac agcagagccc tcgatgtccc
  4561 acttctgctt ccgttccctt tctagaagat tgaaaaaaag gtcaaaacca catgcctgtg
  4621 gagaaagtgc gacatgttta gaaatactgg tagggaacca ggagtaagaa aagctttacc
  4681 agcttttact acaaatggat gaaagacatc aggatcccac caccgcaagg taaagtgact
  4741 tcccttttct ggaacccctg tggcacagga gtaccaattt tcctttccaa cgaactggat
  4801 ttctggatag gcattttggc tgtatgtgga cagataagac cacagtcctt agcccaatcc
  4861 cagctataca gtcaccccaa tttccacaaa tgatgtgatg gtaccgtata atcctgtaat
  4921 tgggaaattt cacatttttc ctgtcctaat ctcagaggtg ggagaagcaa gtctagaaca
  4981 tctccaggct cagactaaac gagagtactt ggactgcaac caagtaatca ctgcaaagta
  5041 gttccaagca gcaagaaata ccagattctc atggaggcta ctatagggta cagaataaca
  5101 acatgaaagc aatcaaccct gtataaataa tgtttcttgg catttttttt ttaattaaag
  5161 aaatccagtg tctcaaaaaa aaaaaaaaaa aaaaaaaaaa aaaaaa
PHC2 (accession No. NM_198040):
(SEQ ID NO: 116)
        1 catctgcctg cccttctgcc atccgagcgc cctgactgcg ccacactgca ggccatggag
      61 aatgagctgc cagtcccaca tacatctagc agtgcctgtg ccaccagcag taccagcggg
    121 gccagtagca gcagtggctg caacaacagc agcagtggtg gaagtggccg ccccaccggg
    181 ccccagattt ctgtgtacag tggtattcca gaccggcaga ccgtgcaggt gatccagcag
    241 gccctgcaca gacagcccag cacggccgct cagtacctgc agcagatgta cgccgcccag
    301 cagcagcacc tcatgctgca gaccgcggcg ctccagcagc agcacctcag cagcgcccag
    361 ctccagagcc tggcagccgt acagcaggca agcctggtat ccaatagaca aggaagcact
    421 tcaggcagca atgtgtctgc gcaggccccg gcccagtcat cttcgatcaa cctggcagcc
    481 tccccagcag cagcccagct cctcaaccgg gcccagagtg tgaactctgc agcagcctca
    541 ggcatcgctc agcaggctgt gctcttgggc aacacgtctt ccccagccct gactgcaagc
    601 caagcacaga tgtatctgag ggcacagatg ctcatcttca cgcccacggc caccgtcgct
    661 actgtgcagc ctgagctcgg cactggctcc cccgcccggc cccccacccc cgcccaggta
    721 cagaacttga ccctccgaac acagcagaca ccagcggcag cagcctcggg ccccaccccc
    781 actcagcctg tcctgcccag cttggccctg aaacccacgc cgggcggtag ccagcctctg
    841 cctaccccag cacagagcag aaatactgct caggcttccc ctgcaggtgc caagcctggc
    901 atagctgaca gtgtgatgga gccacacaag aaaggagatg gcaacagcag tgtgccaggg
    961 agcatggaag gccgggctgg gctcagccgg acggttcctg ctgtggctgc ccaccccctc
  1021 attgcaccag cctatgctca gctgcagcca caccagctcc tcccacagcc atcctcaaag
  1081 cacctgcagc cccaatttgt gatccagcag cagccacagc cacaacagca gcagccgccg
  1141 ccccagcagt cacggcctgt gctccaagct gagccccacc cccagctcgc ctcagtctct
  1201 ccaagcgtgg ccctccagcc cagctcagag gcccatgcca tgccactagg cccggttaca
  1261 cccgccctgc cactccagtg tcccactgcc aacctgcaca agcctggcgg cagtcagcag
  1321 tgtcaccctc ccacacctga tactgggcct cagaatggac atcccgaggg cgtgccccac
  1381 acccctcaac gcaggttcca gcacacttca gctgtcatct tacaactgca gcctgcttca
  1441 ccaccccagc agtgtgtccc tgatgactgg aaagaagtgg caccagggga gaaaagtgtg
  1501 cctgagacgc ggtctggccc atcaccacat cagcaggcta ttgtcactgc catgcctggt
  1561 ggcctgcctg tacccacgag ccctaacatc cagccgtccc cagctcacga gacagggcag
  1621 ggcattgttc atgcactgac cgacctcagc agccccggca tgacctcagg gaacggaaac
  1681 tctgcctcca gcatcgccgg cactgccccc cagaatggtg agaataaacc accacaggcc
  1741 attgtgaaac cccaaatcct gacgcatgtt atcgaagggt ttgtgatcca ggagggggcg
  1801 gagcctttcc cggtgggacg ctcgtccctg ctggtgggga atctcaagaa gaagtatgca
  1861 caggggttcc tgcctgagaa acttccacag caggatcaca ccaccaccac tgactcggag
  1921 atggaggagc cctatctgca agaatccaaa gaggagggtg ctcccctcaa actcaagtgt
  1981 gagctctgtg gccgggtgga ctttgcctat aagttcaagc gttccaagcg cttctgttcc
  2041 atggcttgtg caaagaggta caacgtggga tgcaccaaac gggtgggact tttccactca
  2101 gaccggagca agctgcagaa ggcaggagct gcgacccaca accgccgtcg ggccagcaaa
  2161 gccagtctgc caccacttac caaggatacc aagaagcagc caacaggcac tgtgcccctt
  2221 tcggttactg ctgctttgca gctaacacac agccaggaag actccagccg ttgctcagat
  2281 aactcaagct atgaggaacc cttgtcaccc atctcagcca gctcatctac ttcccgccgg
  2341 cgacaaggcc agcgggacct ggagctcccc gacatgcata tgcgggacct ggtgggcatg
  2401 ggacaccact tcctgccaag tgagcccacc aagtggaatg tagaagacgt ctacgaattc
  2461 atccgctctc tgccaggctg ccaggagata gcagaggaat tccgtgccca ggaaatcgac
  2521 gggcaagccc tgctgctgct caaggaggac cacctgatga gcgccatgaa catcaagctg
  2581 gggcccgccc tgaagatcta cgcccgcatc agcatgctca aggactccta gggctggtgg
  2641 cagccaggat tctggcccag ggcgcctcct cccgactgag cagagccaga cagacattcc
  2701 tgaggggccc agaaatgggg ccggttggag ggcaggggct ctccctaggg gcatagctgg
  2761 tgaggaggtc tgggcacctc ctccatggct ctcaggggcc tttcatttct gtgggagggg
  2821 cagagaggta ggtggcacag aagatggggc tttatgcttg taaatattga tagcactggc
  2881 ttcctccaaa gtcccaatac tctagccccg ctctcttccc ctctttctgt cccccatttt
  2941 ccagggggta tatggtcagg gctccccaac ctgagttggg ttacttcaag ggcagccagc
  3001 aggcctggat ggaggcctag aaagcccttg ccttccttcc tcccacttct ttctccaggc
  3061 ctggttaact cttccgttgt cagcttctcc cccttcagcc tgtttctgca gcagccaggg
  3121 ttctcccccc tacaccctct gcaggtggag agagagaagc tgggcccagc cgggccgtgc
  3181 ctgctggcac agacgcctta acgctgtgtg tatgactgtg tgactgtgtg ggagcctgga
  3241 ctgacagata ggccaagggc tactctctgg catctccagg tgttttgtag caaacagcca
  3301 cttagtgctt tgtcctggac tccactcagc ctcaggatgg ggaatagcca agaatggcag
  3361 cctcagcgca gaggcaaggt cagaaagaga cggcgcttca gagtttcctt tccagacacc
  3421 cctccccgca ctgtgaagtt cccctgaccg ccctcctggt tcacaaagag cattaagaaa
  3481 gctgcggtgg tctgagcaac atagcccaaa gggctgagcc tcctggcctg cctgcccgcc
  3541 caccctggga gtcccagtgg tgaggctcag agaactgcta aggggaaaga acagctggag
  3601 tttctgttga tgtgaagaag gcagctcttg gcctcccact cccacacttc tttgcctata
  3661 aatcttccta gcagcaattt gagctacctg aggaggaggc agggcagaaa gggcgagggc
  3721 ctgcctctga cctgccgtgt cctttgcagg aaggaggtag gcacctttct gagcttattc
  3781 tattccccac ccacaccccc aggcagggtt ggaaatgaag gactttttta acctttgttt
  3841 tgttttttaa aaataaatct gtaaaatctg tct
PHC3 (accession No. NM_024947):
(SEQ ID NO: 117)
        1 atgcgcagcc catgttagtg atggaggaga gaagatggcg gaagcggaat ttaaggacca
      61 tagtacagct atggatactg aaccaaaccc gggaacatct tctgtgtcaa caacaaccag
    121 cagtaccacc accaccacca tcaccacttc ctcctctcga atgcagcagc cacagatctc
    181 tgtctacagt ggttcagacc gacatgctgt acaggtaatt caacaggcat tgcatcggcc
    241 ccccagctca gctgctcagt accttcagca aatgtatgca gcccaacaac agcacttgat
    301 gctgcatact gcagctcttc agcagcagca tttaagcagc tcccagcttc agagccttgc
    361 tgctgttcag gcaagtttgt ccagtggaag accatctaca tctcccacag gaagtgtcac
    421 acagcagtca agtatgtccc aaacgtctat caacctctcc acttctccta cacctgcaca
    481 gttaataagc cgttcccagg cttccagttc taccagcggc agtattaccc aacagactat
    541 gttactaggg agtacttccc ctaccctaac ggcaagccaa gctcaaatgt atctccgagc
    601 tcaaatgctg attttcacac ccgctaccac tgtggctgct gtacagtctg acattcctgt
    661 tgtctcgtcg tcatcgtcat cttcctgtca gtctgcagct actcaggttc agaatttaac
    721 attacgcagc cagaagttgg gtgtattatc tagctcacag aatggtccac caaaaagcac
    781 tagtcaaact cagtcattga caatttgtca taacaaaaca acagtgacca gttctaaaat
    841 cagccaacga gatccttctc cagaaagtaa taagaaagga gagagcccaa gcctggaatc
    901 acgaagcaca gctgtcaccc ggacatcaag tattcaccag ttaatagcac cagcttcata
    961 ttctccaatt cagcctcatt ctctaataaa acatcagcag attcctcttc attcaccacc
  1021 ttccaaagtt tcccatcatc agctgatatt acaacagcag caacagcaaa ttcagccaat
  1081 cacacttcag aattcaactc aagacccacc cccatcccag cactgtatac cactccagaa
  1141 ccatggcctt cctccagctc ccagtaatgc ccagtcacag cattgttcac cgattcagag
  1201 tcatccctct cctttaacag tgtctcctaa tcagtcacag tcagcacagc agtctgtagt
  1261 ggtgtctcct ccaccacctc attcaccaag tcagtctcct actataatta ttcatccaca
  1321 agcacttatt cagccacacc ctcttgtgtc atcagctctc cagccagggc caaatttgca
  1381 gcagtccact gctaatcagg tgcaagctac agcacagttg aatcttccat cccatcttcc
  1441 acttccagct tcccctgttg tacacattgg cccagttcag cagtctgcct tggtatcccc
  1501 aggccagcag attgtctctc catcacacca gcaatattca tccctgcagt cctctccaat
  1561 cccaattgca agtcctccac agatgtcgac atctcctcca gctcagattc caccactgcc
  1621 cttgcagtct atgcagtctt tacaagtgca gcctgaaatt ctgtcccagg gccaggtttt
  1681 ggtgcagaat gctttggtgt cagaagagga acttccagct gcagaagctt tggtccagtt
  1741 gccatttcag actcttcctc ctccacagac tgttgcggta aacctacaag tgcaaccacc
  1801 agcacctgtt gatccaccag tggtttatca ggtagaagat gtgtgtgaag aagaaatgcc
  1861 agaagagtca gatgaatgtg tccggatgga tagaacccca ccaccaccca ctttgtctcc
  1921 agcagctata acagtgggga gaggagaaga tttgacttct gaacatcctt tgttagagca
  1981 agtggaatta cctgctgtgg catcagtcag tgcttcagta attaaatctc catcagatcc
  2041 ctcacatgtt tctgttccac cacctccatt gttacttcca gctgccacca caaggagtaa
  2101 cagtacatct atgcacagta gcattcccag tatagagaac aaacctccac aggctattgt
  2161 taaaccacag atcctaaccc atgttattga aggctttgtg attcaggagg gattggagcc
  2221 atttcctgtg agtcgttcct ctttgctaat agaacagcct gtgaaaaaac ggcctctttt
  2281 ggataatcag gtgataaatt cagtgtgtgt tcagccagag ctacagaata atacaaaaca
  2341 tgcggataat tcatctgaca cagagatgga agacatgatt gctgaagaga cattagaaga
  2401 aatggacagt gagttgctca agtgtgaatt ctgtgggaaa atgggatatg ctaatgaatt
  2461 tttgcggtca aaacgattct gcactatgtc atgtgccaaa aggtacaatg ttagctgttc
  2521 taaaaaattt gcacttagtc gttggaatcg taagcctgat aatcaaagtc ttgggcatcg
  2581 tggccgtcgt ccaagtggcc ctgatggggc agcgagagaa catatcctta ggcagcttcc
  2641 aattacttat ccatctgcag aagaagactt ggcttctcat gaagattctg tgccatctgc
  2701 tatgacaact cgtctgcgca ggcagagcga gcgggaaaga gaacgtgagc ttcgggatgt
  2761 gagaattcgg aaaatgcctg agaacagtga cttgctacca gttgcacaaa cagagccatc
  2821 tatatggaca gttgatgatg tctgggcctt catccattct ttgcctggct gccaggatat
  2881 cgcagatgaa ttcagagcac aggagattga tggacaggcc cttctcttgc tgaaagaaga
  2941 ccatctcatg agtgcaatga atatcaagct aggcccagcc ctgaagatct gtgcacgcat
  3001 caactctctg aaggaatctt aacaggaaca tgaagccttg ataaaacagc agttttactt
  3061 ttctcacaaa aacttgtaag gtaaaggcct aacttggtct agaatatgac acttattgtg
  3121 gtggatagcc aagcacattg ggatctccac atcaaatact gacatttctt ctacaggtat
  3181 aataattcat catgcatttt cataattaat aaacattggt aaaattaatt ttacaggtta
  3241 catgaaacat tgaaagactt gttacagagg gccatgatat ttttcaaaga aatgtgttat
  3301 actagataat ttttttaaag gtgatgttta tcattaatat aaagaatcct tttaaaagta
  3361 atttaatgat ttacatttct cctcttttga ttcaattttc ttatacattt tttctaccct
  3421 attagttttc taaaggttgt catgagaggt atattatgga ataatttagt agtccagtga
  3481 cagaatcgta tgaaatcagt gtacatttta aaaaacatgt cttttagaca tatgctttat
  3541 ctataaaaaa ggaattgtgt tctagtatga acaatactga tctggaagtg agaagagtta
  3601 gtttctattc caaacttgac caagaatttg gtttgactga gaacgttttc ctctcagttt
  3661 ttgtacattt atttagagca gtggttctca gtggaggtca gttttgatcg ccaggggaca
  3721 tctggcaatg ttgagacatt ttggttgtca cagcttgggg gtgggttcag gggagggttg
  3781 ctactggtgt ctagtagtta gaagccagag atgtttctaa acatcttata atgcacagga
  3841 cagcacccct ccactgtaaa gaattattgg ttcaaaaata tcggtactgc caaggttgag
  3901 aaactctgat atagaaggag tgataaatat tgttttcacc caaaggaata cttttaaagg
  3961 atgaagctta ctaaacatat atgatggaag tattattcag ataacattaa tattctgctg
  4021 aataattttt tctagtttaa tcatactaga aaaagaaaaa aaatctacaa attgtcctat
  4081 aaaataagga caaacatgca aataatttaa ctctcagaaa gtactaattc attctgatta
  4141 tctttcatac ctctgtgctc ctctgcactg acgaagacat aatatgatta tacctatgaa
  4201 ctagtgcaca gccttttctg gcaagaaaat agtttgtagc agatacgtgg ttgctctttg
  4261 gatttttttc tattgttgaa catgctggga ctagctagaa tgcacattcc tacttccttt
  4321 accaaacgtt tgcatgcttc ctgcaaagca cttaccaagt gatttctctt gaaccatcgg
  4381 atataatttt gtatgtacat gtttgaggaa aaaaatgtaa agcaaaacct tttactgaac
  4441 agtgttctat agaattatga cactaaaaca aaattgtttg tggaagccct gaaagcttta
  4501 tagtcctgga catcaaaaat tttatttgag atgatgaatg ttttgttttc atcttttctt
  4561 atattaccac aattgagata ttttagtaat tgaaggaaca tacacagata tttggcagaa
  4621 gtcgagtaag gaggggaaaa aaagagtccg tgagtttcag tcattttcac tgctcttttc
  4681 aaaaagattg tgttgagctg gtagaagact aaagatgtca ctgaagacat cacagatact
  4741 atatttatct tttggctttg tgtacattag agaatgttga ttatttttat acaaaaatac
  4801 agcgggtaat ttttttaatc tttagatgcc tcttgtttga atgtatgctt tgtggaattc
  4861 tttgtgtagt aatgttttaa aaaaagatgt ttactgatag ttacatgtag gattagaata
  4921 tgtaatataa tataaggctc atgttccaga cctacgatag cttgtagtct atgttacgta
  4981 tttctttata tcacattttt aatcattgga ttaaagtatc aaggaaagct aggtactcta
  5041 taatgagttt tcatttatta gcagttaatc atcatgacag aattgtcata tgcttgactt
  5101 ttccctcttc ttggaatttc agaacacaaa tacaggctaa gcattagtaa gagatggccc
  5161 acagtatgag agagagaggt gcaacggaaa atctcgcctg gaattaaaac ttttcataga
  5221 ttatccacgg ttaatacaaa atttattata tggggataga ctgctccagc aataatgatt
  5281 acatcctata actgtattac ctatggcctt taaggtatca attttgaact gtgttgtagg
  5341 ctctcctttt atttgttctc tttcctaata gcagccattc tgtacttatt gaaagcccct
  5401 gtgcctactg ctgtcttaag tattcaggag gggcttacaa gagggttttc tattggagaa
  5461 taccgtataa tcttaaatct agtccagatc tctgttgtcc ccactcaaaa catacacaaa
  5521 atatgcactt gcttttttca agtgagtttt tatttaaaaa tggcttgttt gctatcacat
  5581 tggtgcagct gtttctttca agatgagtta atcatcttaa tttcaaagct tcagctatat
  5641 ataatggata tatagacaac actgagcatc cacctctctc ctgagcttta aagcagagtt
  5701 tcagtatgat ataggtgggg agagtaaatt gttttcatat cctttcatac tactactaat
  5761 agttttagga ttttgactgg ggagagataa tgacaaacag aaagggaaca tggaggttct
  5821 tcctactttt gctacctaag tttgcatttt ctgacttcct tgcagtgttg cactctttgt
  5881 cccattggga taaaaagcat aagtttgaaa ttttgcttta agccttgtgt tcctggggaa
  5941 gttaaacaac taagagagct gatttgtaaa aattattttt tatatgacat taatattcat
  6001 caagccttgt gtaggcatgt gtaagacaca gctatgcagc tttgagtagt caatatagta
  6061 tgagatagag tgttgtccca aatcctcctg tcacttttta agtagcatat tatttccctg
  6121 atggtcctgt tactttgctg ttgaatgctc taaacagaac tttttaaaag gtgtgtttta
  6181 agagcagtca cctaggagta gacaaggtgg aatgggagga gagaaatggt aatgcaaaag
  6241 cttgagcatg ggaagagtca gaggaggagg ccatcatcct tgttagctta gcctacttca
  6301 acactgagca catttctgca cttttgaagt gaaattcatg ttttacttag aagaaataat
  6361 tttctttcat tagggatccc agttgatttt tgtttcctgg tgtatcaaaa tacttagaac
  6421 tatgaaacaa gtattattgt gatcatgcct ttgaataatt tttgacgtag cttatcttca
  6481 tgtatcaagt ataaaattat aatgagacat ctattcacaa atacaagtct tagattgaat
  6541 tgaaatgtgt tatagtgccc tgtctcccac tgacttgttc agttaaatgt cttaaagtac
  6601 attatgtaca tcttcaggct tttggtacca caatggcaca agtatggtag ggaggcaata
  6661 tagtcttagg ctatatgcct atattaagtg tgtataaaca atttttgaaa gaatacacta
  6721 ttatagatgt atgtgagtga tgctgacctg acagccatat ccagtggatg aaactgactg
  6781 gacacactgt taaaatgttt taaagatgta ttttcagcca gaacagcctg gttatagttt
  6841 gtggttttca ccttggtgga ttgcaggaac acatgcagcc tactggcatt gagcattagc
  6901 taatggcatg aaagggcctc atctcactac ctctctaagg cctctagctc caagaaaacc
  6961 atgaaaactt ctttcttgga gagatctttg tctcagaatc cttagagagg atttcgtatg
  7021 ggggctaact ttaggaaggg aggcagctgg ggcaggactt tctgatacct gacagtcatg
  7081 ttccagagca acctttgggc agtggaaact ggcgcatcta tgcaaaatga ttgctcaatc
  7141 tctatcttgt gtactacata tgtaactagc tgggccctaa ggaaggtttt ctagggggaa
  7201 ggatagggaa gtagaggagg agacaagtag gaggaacaaa gcattctaga cccaagagga
  7261 tagaagatat ttaggataga tatggctttc atccatagtt caaaataatg cgttttgtta
  7321 gatgccagtt atagcagtaa ataggttata gtttttatat gtcaagattt acctgtaatc
  7381 agactcattc tttcactctc tatacccact gtctccatgc ttgggagcat ggatattaat
  7441 agttccagtg atgtagaagt tagtgatttt tgatttctga aaaaggtgag aaccttttat
  7501 tacagttgga gaatatttgt caaaaattca aaggttgttg taattgagtt gccagaatta
  7561 cagagtttcc attttcagat atcacagttg aatcacctct gtagattgtt ataaagagag
  7621 gcattttaag atagtatttt atttgctagg ttgtgtctca gtctaagaat tgggaaaaga
  7681 agagctatag gtttctcttt cctagtctgg atttcagtaa acacaagcct acctctgctt
  7741 ctttggttca cagcagtgtg gatcatgaaa tgaactgttt acccacattc atcaatattg
  7801 gtattttaca aatctacttg gagcatttaa tttcatctca aagattgtga tccactttag
  7861 ataagcacaa atacagtatt aggaaaagta aatatgcaat cttactaaaa tttcaacttg
  7921 ttaagctgta tatcttaaaa gaaattattt ggggctgggc atggtggctc acacctgtaa
  7981 tcccagcact ttgggaggct gaggtgggta gatcacctga ggtcaggagt tcgagaccag
  8041 cctgaccaat atggtgaaac cctatctcta ctaaaaacac aaaaattagc tgggtgtggt
  8101 ggcatgcacc tgtaattcca gctacttggg aggctgagac aggagaattg cttgaaccca
  8161 ggtggtggag gttgcagtga gccaagatca cacccctgca ctccagcctg ggtgacagag
  8221 cgagactcca tctcaaaaaa acaaaacaaa aaattatttg ggaagatacg tcctctttta
  8281 ttagaagttc ataaaatgta tcatatagtt ttgttcacag tagttatata agctttcttc
  8341 aaataaattt aaaattagat taccttcttt ggaaaaagaa tttcctaaat ttttaagaat
  8401 tttcaaagtt ttacatatta gtttttagaa cctaatccgt tttaaaattg tactatgaga
  8461 aagctttttt ttgaaagttg taaagcatta atacaaataa tacaaatata attattacca
  8521 tcacattcca gagaatatgg ctttttctaa actttcaatt tagaaaacat acattaaggg
  8581 agaatctctg ccctcctttt cagctctgaa gatcagcttt tctactcaga cacatgcaca
  8641 caccccttcc aagtgtcatg tttatgggaa catttgggaa atgttttcca gatgttttat
  8701 tttttccctt ttatagtttg ttgacattta attttactta aagatgacaa ttttaatcgg
  8761 aaatgttaga ggtacaacat agtgaggttc tagctagctt tatacttttg aaaaatattt
  8821 ttgtttctac tgctttttac aagtactagt cctctcagtg atactggtgg tgttcagtat
  8881 gaatccatag aaagaaaaca aaatttgttg tttaaaaaaa gcagagtaat gaatgaattt
  8941 cagttttgaa aacaacataa tttgaaaaca ctgttatact aacatggcaa ggtgttaatt
  9001 aaatataaga gtaaggtagt aagttctttt agagcacctg tttaaattta ctccagtaat
  9061 catcttaagg attgatagtc accatcactt attggcttaa aagttatatt tcatggaata
  9121 ttatcagtgt taaatccaag ctttgtggag ctttaagtga tggtggtgaa aaagttggtg
  9181 tttatgagag agtggtgggg tgtctagtca ttagtgaagt taaacatcaa cctgttttag
  9241 aaagaatttt ttagtcttgc ctaaagtaaa ccagaagtgt ctagtgttta aatctttatt
  9301 tagaatgctt ctcttaaaag tattttttgt tttgggtagt attaaataat cagtaaataa
  9361 tctatttcag tagtaaataa tgaattaaga tgatgatgaa tgaggattaa cacactggtc
  9421 tggagactgg ggttttattt cagtgggtta gctgtgtgtg acatgttggg caattactca
  9481 gctgttttaa cagcttccag atatgcagta tggtgcctgt actactcaaa agttgatttt
  9541 ggtttaattc atctttaagg tacctcccag ctctaaaact atgattctag gctgtgtaat
  9601 ggggttattc ctactttatt ctctttcctt ttttaagggt tcattttata cttaataagc
  9661 atccatttct tgggtcacct acagtctttg ttctcctaag gattaaaata gaaaattcat
  9721 acataacaag caaatgatga cattttccta aatgctcctt attggttaac cactgaatat
  9781 atgaacacat atgaatattg tcattcatgt acttaaattc atttagcaaa ctatttgaac
  9841 acttacatgt gcagtgtttg gtgaacatga catgaggaac tagtagtaag taaaatcttc
  9901 cccccaaaat tcattgtggc ttaaataaat atgaacataa tcattactac ttaatatact
  9961 gagagggaat cttaataaac ttggaactgg gagggaatat ttgtatacat tgggtaaagg
10021 gttaggctag atgacatcta aggggtctga gtgaatcata tcataatttt tataacacat
10081 ttcacatact aaacatcagt tggccccata cctgattaag ttacaaaatt taggagactt
10141 aacattaagg acttacaggt tgagacagcc cgtatttcac aacattattt tgacacttga
10201 ctctattcca gagttgttgc tatacaaggc atgtggcaga acaaaaaaaa agctggtgtt
10261 gatataagag ctttttaccc agtattgaca gtgagcaact ttctttcttt tttttttttt
10321 ttcttttttt tttttttgag atgggttcgc tctgttgccc aggctggtgt gcagtggtgc
10381 gatctcagct cactgcaacc tccacctccc gggttgaagc gattgtcttg cctcagcctt
10441 ccaagtagct ggaattacag gtgcccgccg ccacacctgg ctaatttttg tatttttagt
10441 ccaagtagct ggaattacag gtgcccgccg ccacacctgg ctaatttttg tatttttagt
10501 agagacgggg cttcaccatg ttggccaggc tagtctcgaa ctcttgacct caagtgatcc
10561 acctgccttg gcctccctaa gtgctgggat tacaggcatg agccaccaca cctgtccgac
10621 agtgtagcaa ctttctaaaa ctgaaaaatc tcaaaggaga tcattggaac tgacttgttc
10681 atttattttt tgtttttaaa ttaagaaaga ttacacaaaa taagtgttac tgtactttaa
10741 gctattacaa atatccaact tttaaagata tgtaagaatc agtaatattc tagaaagcac
10801 atatatagta aaagggcatc ctttaaatgt agaacgggta aacatgaaac agttccatgc
10861 ttgaattgtt aagtatctag ggggtaaaca ttgaatggga gaatcattta ttgggttaag
10921 gtcccttcct tgtcattctg ggatctgtga atcacattgt aattcctgtt gacaaagctt
10981 tacttgttaa catcagttga tactgacatt ctccataaag atatagaatg aaaatatcta
11041 ttaaaaatag tttatcattg ttttagcttt tttgttttgt ttgttttgag acagagtctc
11101 actgtcaccc aggcttgagt gcagcggtgt gatcttggct caatgcaacc tccacctccc
11161 aggttcaata gattctccca ccttggcctc ccaagtagct gggattactg gcatgcacca
11221 ctatgcctgg ccagtttttt gtatttttag tagagatggg gtttcaccat gttggccagg
11281 ctggactcga actcctgacc tcaagtgatc cgcccacctc agcctcccaa agtgccggga
11341 ttataggcat gagccactgc gcctagcctg ttgcagcttt ttaaagcagg aaaatatcca
11401 tataaactgt tgggttagaa tctatattag aatctttcaa actaattgaa aacaggaaga
11461 ctatcatcta agtagccaga taatctgggt ttcaaaaagt tattccatgg tactggttta
11521 aaaaatactt ttcaagtgtt ttaattttta aagtgtaact aattcttcaa atatgttatg
11581 ctgttaaaat atgtattcca taagtacttt ttgtatatgt attcttaaat tttaaaaagt
11641 caactgaatg cgcaaagatg atataatttt ggatgtagac atttaaacta gattcccagt
11701 cctctccttc aaaagcttgg tctttgtttt tcctataggg aaaaaagtca aaataagttc
11761 caaaaactat cctcaaagta gtattgtgct tgtagtaaat gaaggttgga tggatggata
11821 ctgacaatgg tggcaggcat ttcaagcctt ttaaattagt actttttgtc gtcttgctta
11881 ttaaaatttt gttaatttta gcaaagacca attgttgtga taaactggtg ttttttggat
11941 gcttcaagca cacgttaacc aattttttaa ttcccctttt ggttcctccc attgttctaa
12001 aataggactt tcatattatt aaaacctcaa aagatgatcc acccaggatg aacaaagatc
12061 accaagggga aagaaaacat tttttatctt tacagaaaac atgttaagat tatatataga
12121 tgtattcttt acattggata ttgtattaga gtcctcctta caagaaatga aatagttttt
12181 agcactctta gcattagagt tcctagattg gtgttgatag ctacagtttt aaaatgtata
12241 acctgaaaat gaaggttaat tttgcattgt aagagcacat ttgatctatg taaaaagtgt
12301 ccatttggtg tattttttta aaaaagagaa agcactttca tattaagtag catgtgtatg
12361 aatttagatt ttcatatttg ttgtgtctgt attcagtgaa gtaaattgag catttaaatg
12421 tttgttgatg gcaacattaa ctattaaatt aaagcacctt atactctgct gcttaacttg
12481 cttgtaattg cacctttgtt acctgcacat tttcatatag aatattgttg taacattgct
12541 tcatgtgggt ctggatggaa gattagtggg cctacaggat catttattta tattgtttat
12601 attacaataa tatattgtag atcagttgta agttcatttc tttacaaata aaagcctctt
12661 ccatttgact ggaaaaaaaa aaaaaaa
BMI1 (accession No. NM_005180):
(SEQ ID NO: 118)
        1 acagcaacta tgaaataatc gtagtatgag aggcagagat cggggcgaga caatggggat
      61 gtgggcgcgg gagccccgtt ccggcttagc agcacctccc agccccgcag aataaaaccg
    121 atcgcgcccc ctccgcgcgc gccctccccc gagtgcggag cgggaggagg cggcggcggc
    181 cgaggaggag gaggaggagg ccccggagga ggaggcgttg gaggtcgagg cggaggcgga
    241 ggaggaggag gccgaggcgc cggaggaggc cgaggcgccg gagcaggagg aggccggccg
    301 gaggcggcat gagacgagcg tggcggccgc ggctgctcgg ggccgcgctg gttgcccatt
    361 gacagcggcg tctgcagctc gcttcaagat ggccgcttgg ctcgcattca ttttctgctg
    421 aacgactttt aactttcatt gtcttttccg cccgcttcga tcgcctcgcg ccggctgctc
    481 tttccgggat tttttatcaa gcagaaatgc atcgaacaac gagaatcaag atcactgagc
    541 taaatcccca cctgatgtgt gtgctttgtg gagggtactt cattgatgcc acaaccataa
    601 tagaatgtct acattccttc tgtaaaacgt gtattgttcg ttacctggag accagcaagt
    661 attgtcctat ttgtgatgtc caagttcaca agaccagacc actactgaat ataaggtcag
    721 ataaaactct ccaagatatt gtatacaaat tagttccagg gcttttcaaa aatgaaatga
    781 agagaagaag ggatttttat gcagctcatc cttctgctga tgctgccaat ggctctaatg
    841 aagatagagg agaggttgca gatgaagata agagaattat aactgatgat gagataataa
    901 gcttatccat tgaattcttt gaccagaaca gattggatcg gaaagtaaac aaagacaaag
    961 agaaatctaa ggaggaggtg aatgataaaa gatacttacg atgcccagca gcaatgactg
  1021 tgatgcactt aagaaagttt ctcagaagta aaatggacat acctaatact ttccagattg
  1081 atgtcatgta tgaggaggaa cctttaaagg attattatac actaatggat attgcctaca
  1141 tttatacctg gagaaggaat ggtccacttc cattgaaata cagagttcga cctacttgta
  1201 aaagaatgaa gatcagtcac cagagagatg gactgacaaa tgctggagaa ctggaaagtg
  1261 actctgggag tgacaaggcc aacagcccag caggaggtat tccctccacc tcttcttgtt
  1321 tgcctagccc cagtactcca gtgcagtctc ctcatccaca gtttcctcac atttccagta
  1381 ctatgaatgg aaccagcaac agccccagcg gtaaccacca atcttctttt gccaatagac
  1441 ctcgaaaatc atcagtaaat gggtcatcag caacttcttc tggttgatac ctgagactgt
  1501 taaggaaaaa aattttaaac ccctgattta tatagatatc ttcatgccat tacagctttc
  1561 tagatgctaa tacatgtgac tatcgtccaa tttgctttct tttgtagtga cattaaattt
  1621 ggctataaaa gatggactac atgtgatact cctatggacg ttaattgaaa agaaagattg
  1681 ttgttataaa gaattggttt cttggaaagc aggcaagact ttttctctgt gttaggaaag
  1741 atgggaaatg gtttctgtaa ccattgtttg gatttggaag tactctgcag tggacataag
  1801 cattgggcca tagtttgtta atctcaacta acgcctacat tacattctcc ttgatcgttc
  1861 ttgttattac gctgttttgt gaacctgtag aaaacaagtg ctttttatct tgaaattcaa
  1921 ccaacggaaa gaatatgcat agaataatgc attctatgta gccatgtcac tgtgaataac
  1981 gatttcttgc atatttagcc attttgattc ctgtttgatt tatacttctc tgttgctacg
  2041 caaaaccgat caaagaaaag tgaacttcag ttttacaatc tgtatgccta aaagcgggta
  2101 ctaccgttta ttttactgac ttgtttaaat gattcgcttt tgtaagaatc agatggcatt
  2161 atgcttgttg tacaatgcca tattggtata tgacataaca ggaaacagta ttgtatgata
  2221 tatttataaa tgctataaag aaatattgtg tttcatgcat tcagaaatga ttgttaaaat
  2281 tctcccaact ggttcgacct ttgcagatac ccataaccta tgttgagcct tgcttaccag
  2341 caaagaatat ttttaatgtg gatatctaat tctaaagtct gttccattag aagcaattgg
  2401 cacatctttc tatactttat atacttttct ccagtaatac atgtttactt taaagattgt
  2461 tgcagtgaag aaaaaccttt aactgagaaa tatggaaacc gtcttaattt tccattggct
  2521 atgatggaat taatattgta ttttaaaaat gcatattgat cactataatt ctaaaacaat
  2581 tttttaaata aaccagcagg ttgctaaaag aaggcatttt atctaaagtt attttaatag
  2641 gtggtatagc agtaatttta aatttaagag ttgcttttac agttaacaat ggaatatgcc
  2701 ttctctgcta tgtctgaaaa tagaagctat ttattatgag cttctacagg tatttttaaa
  2761 tagagcaagc atgttgaatt taaaatatga ataaccccac ccaacaattt tcagtttatt
  2821 ttttgctttg gtcgaacttg gtgtgtgttc atcacccatc agttatttgt gagggtgttt
  2881 attctatatg aatattgttt catgtttgta tgggaaaatt gtagctaaac atttcattgt
  2941 ccccagtctg caaaagaagc acaattctat tgctttgtct tgcttatagt cattaaatca
  3001 ttacttttac atatattgct gttacttctg ctttctttaa aaatatagta aaggatgttt
  3061 tatgaagtca caagatacat atatttttat tttgacctaa atttgtacag tcccattgta
  3121 agtgttgttt ctaattatag atgtaaaatg aaatttcatt tgtaattgga aaaaatccaa
  3181 taaaaaggat attcatttag aaaatagcta agatctttaa taaaaatttg atatgaaaag
  3241 cacaatgtgc agaagttatg gaaaacctat agaggattac aacaggtaaa cgttaaagag
  3301 aatacattgc tgacttatag tgatgtggct aagaagtaca tgctttgttg taaaattgct
  3361 tgaaagccca ttgaaagatg tatctgttta tttacagtct ttgaagtaaa agttaccaat
  3421 gtttgccaat aaaaa
PCGF2 (accession No. NM_007144):
(SEQ ID NO: 119)
        1 tctcccctcc cgccgcccgg gcgagcgaca cggctgcggc ccccctcccc tcccttccct
      61 ccctccctcc catccccccc tccccgagac ccaccggacc ccgaacccag atggccgaaa
    121 cgggctcccc gtcttaacga tttggcgtct ccctcgacca ccacctcttt gtgcagcagc
    181 ccccgggcag accctgttcc gagggcaacg ctccccagtc cccccacccc cgaccccgga
    241 atcatgcatc ggactacacg gatcaaaatc acagagctga acccccacct catgtgtgcc
    301 ctctgcgggg ggtacttcat cgacgccacc actatcgtgg agtgcctgca ttccttctgc
    361 aaaacctgca tcgtgcgcta cctggagacc aacaaatact gccccatgtg tgacgtgcag
    421 gtccataaaa cccggccgct gctgagcatc aggtctgaca aaacacttca agacattgtc
    481 tacaaattgg tccctgggct ttttaaagat gagatgaaac ggcggcggga tttctatgca
    541 gcgtaccccc tgacggaggt ccccaacggc tccaatgagg accgcggcga ggtcttggag
    601 caggagaagg gggctctgag tgatgatgag attgtcagcc tctccatcga attctacgaa
    661 ggtgccaggg accgggacga gaagaagggc cccctggaga atggggatgg ggacaaagag
    721 aaaacagggg tgcgcttcct gcgatgccca gcagccatga ccgtcatgca tcttgccaag
    781 tttctccgca acaagatgga tgtgcccagc aagtacaagg tggaggttct gtacgaggac
    841 gagccactga aggaatacta caccctcatg gacatcgcct acatctaccc ctggcggcgg
    901 aacgggcctc tccccctcaa gtaccgtgtc cagccagcct gcaagcggct caccctagcc
    961 acggtgccca ccccctccga gggcaccaac accagcgggg cgtccgagtg tgagtcagtc
  1021 agcgacaagg ctcccagccc tgccaccctg ccagccacct cctcctccct gcccagccca
  1081 gccaccccat cccatggctc tcccagttcc catgggcctc cagccaccca ccctacctcc
  1141 cccactcccc cttcgacagc cagtggggcc accacagctg ccaacggggg tagcttgaac
  1201 tgcctgcaga caccatcctc caccagcagg gggcgcaaga tgactgtcaa cggcgctccc
  1261 gtgcccccct taacttgagg ccagggaccc tctcccttct tccagccaag cctctccact
  1321 ccttccactt tttctgggcc cttttttcca cctcttctac tttccccagc tcttcccacc
  1381 ttgggggtgg ggggcgggtt ttataaataa atatatatat atatgtacat aggaaaaacc
  1441 aaatatacat acttattttc tatggaccaa ccagattaat ttaaatgcca caggaaacaa
  1501 actttatgtg tgtgtgtatg tgtggaaaat ggtgttcatt ttttttgggg ggggtcttgt
  1561 gtaatttgct gtttttgggg gtgcctggag atgaactgga tgggccactg gagtctcaat
  1621 aaagctctgc accatcctcg ctgtttccca aggcaggtgg tgtgttgggg gccccttcag
  1681 acccaaagct ttaggcatga ttccaactgg ctgcatatag gagtcagtta gaatcgtttc
  1741 tttctctccc cgtttctctc cccatcttgg ctgctgtcct gcctctgacc agtggccgcc
  1801 ccccacgttg ttgaatgtcc agaaattgct aagaacagtg ccttttacaa atgcagttta
  1861 tccctggttc tgaggagcaa gtgcagggtg gaggtggcac ctgcatcacc tcctcctctt
  1921 gcagtggaaa ctttgtgcaa agaatagata gttctgcctc tttttttttt tttcctgtgt
  1981 gtgtggcctt tgcatcattt atcttgtgga aaagaagatt caggccctga gaggtctcag
  2041 ctcttggagg agggctaagg ctttagcatt gtgaagcgct gcacccccac caaccttacc
  2101 ctcaccgggg aaccctcact agcaggactg gtggtggagt ctcacctggg gcctagagtg
  2161 gaagtggggg tgggttaacc tcacacaagc acagatccca gactttgcca gaggcaaaca
  2221 gccttccaat tgcccctcca cccccagctg aggcccggtc acctggtcag gacagagcaa
  2281 ctgcatctaa aagcacaaga agacagaaac ctgtaagctc tgaccccacc cccacccctt
  2341 gagaggtcag cggaccacct ccttagggac agaccctggc aggtcgctgc ccaccgagat
  2401 ttcctcaagt gtgcatagat ctgagaggag tcgggagtcg agactcgaga ttccatcata
  2461 gcgtaggtgt gtggggttgg gagccccctg atgggcttgt ctgtgtttgc accttgtcct
  2521 gtgtctgagg tcctgtgact gtaccctcct ttgccctggg acatctgtat ctcttggctt
  2581 tgtaataaat gctgcatact ttctaaaaaa aaaaaaaaaa aa
ZNF134 (accession No. NM_003435):
(SEQ ID NO: 120)
        1 cgaactgtga tggcggcggc cgcggtgatg ggcccggcgc agatcctctg tggttgttga
      61 attgtaacaa gagagagaac tctggctgcc tgagagggca tgactctagt cacagcagga
    121 ggggcttgga caggccctgg ttgttggcat gaagtgaagg atgaagagtc atcttctgaa
    181 cagagcattt ctatagcagt gtcacatgtt aatacttcca aggcaggttt gcccgcacag
    241 acggctctcc cttgtgacat atgtggcccc atcttgaaag atattttgca cctggatgaa
    301 caccagggta cacaccatgg actgaaactt cacacatgtg gggcatgtgg gagacaattc
    361 tggttcagtg caaaccttca tcagtaccag aagtgttaca gtatagagca acccttaaga
    421 agggataaaa gtgaggcctc aattgtgaag aactgcacag ttagcaaaga acctcatccg
    481 tcagagaagc cctttacgtg taaggaggag cagaaaaact tccaggctac tttgggtggc
    541 tgccaacaaa aggccatcca cagtaagagg aagacacaca ggagcactga gagtggggat
    601 gcatttcatg gtgaacaaat gcattacaag tgcagtgaat gtgggaaagc tttcagccgc
    661 aaagacacac ttgtccagca ccagagaatt catagtggag agaagcctta tgagtgcagc
    721 gaatgtggga aagccttcag ccgcaaagct acacttgtcc agcatcagag aatccatact
    781 ggagaaaggc cttatgaatg cagcgaatgt ggaaaaacct tcagtcgaaa agacaacctt
    841 actcagcaca agagaatcca cactggagaa atgccttata agtgcaatga atgtgggaaa
    901 tattttagcc atcactccaa tctaattgta caccagagag ttcacaatgg agcaaggcct
    961 tataagtgca gtgattgtgg gaaagtcttc agacacaaat ctacacttgt tcagcatgag
  1021 agtattcaca ctggagaaaa tccttatgat tgcagtgatt gtgggaaatc ctttggccac
  1081 aaatacaccc tcattaaaca tcagcgaatt cacactgagt caaagccgtt tgagtgcatt
  1141 gaatgcggga aattctttag tcgaagttct gactatattg cacaccagag ggttcacact
  1201 ggtgaaaggc cttttgtgtg cagtaaatgt gggaaagact ttatcagaac ctcccacctt
  1261 gttcgacacc aaagagttca cactggagaa aggccatatg agtgcagtga atgtgggaag
  1321 gcctacagct taagctccca cctcaatcgg caccagaaag ttcacactgc aggcaggctt
  1381 taggagtgct ttgaatacaa caggactcat caatcagatg ttgaatttca tgtatctgaa
  1441 cattgacaca aaggagatac cttatggtgc caggtacgtg ggaaccttct agggatatgt
  1501 tgcactttct gacttgctca ggttttttgc cagagttatg tcactgtcaa tccatgtggc
  1561 cgaaaccatc ttaactctac cagctaagat accccagcat tggggaaggc agggttttgt
  1621 attgtccagt ccctggagaa aatcatgaaa tgcctgagtt cattgggggt cctcattccc
  1681 ttctgtatga caggtatagg tatggatatg acccattttt agccaagagg gtctgagctg
  1741 tatctgctgg tggcttatac aaaaagttta ctttcttcat ggatattctt ggtctcacat
  1801 acttgtaatc aagtttttcc agcctccaag tcacctggcc tgggaaagta cttgcctcat
  1861 gttgctctgg tttgtgataa taaaggcttt acagtttaag ccacatttaa tcttggggct
  1921 tcttcttatg gtctggggtg gattgaaaac aggctctgcc aaactgaaga cagcctttgt
  1981 gcggtgcctc caactttgcc tcaaatggga cagtgggttg agggagaaca gttcttagtc
  2041 cagttttgat gttaacttcc atagctgaca aagcttgtta agtaagaatt aagatcttgt
  2101 gtagacctga tttgtctgga ttttagagtt atttgagagc ccatatttca ccttgaggag
  2161 ggtgctgctg ctgtgacagc ctgcagtgtt ttgaaacagc atggattggg tgtcttgttt
  2221 gcagcatgtg tcccatgttc cccaacac
RING1 (accession No. NM_002931):
(SEQ ID NO: 121)
        1 cagcgcccgg gccatggcgg cggcggtggc gggagctgct gtctgagcag cggttgcgga
      61 ccgagcgaac ttggcccagg agcccgggcc tagggagagg cgcggcggcg gcgggagcgc
    121 gaacggctgg agctggcctt cttcgccttc tcctcggctg tggagccctg gtggggggtc
    181 tgcgcccggt caccatgacg acgccggcga atgcccagaa tgccagcaaa acgtgggaac
    241 tgagtctgta tgagctgcac cggaccccgc aggaagccat aatggatggc acagagattg
    301 ctgtttcccc tcggtcactg cattcagaac tcatgtgccc tatctgcctg gacatgctga
    361 agaatacgat gaccaccaag gagtgcctcc acagattctg ctctgactgc attgtcacag
    421 ccctacggag cgggaacaag gagtgtccta cctgccgaaa gaagctggtg tccaagcgat
    481 ccctacggcc agaccccaac tttgatgccc tgatctctaa gatctatcct agccgggagg
    541 aatacgaggc ccatcaagac cgagtgctta tccgcctgag ccgcctgcac aaccagcagg
    601 cattgagctc cagcattgag gaggggctac gcatgcaggc catgcacagg gcccagcgtg
    661 tgaggcggcc gataccaggg tcagatcaga ccacaacgat gagtgggggg gaaggagagc
    721 ccggggaggg agaaggggat ggagaagatg tgagctcaga ctccgcccct gactctgccc
    781 caggccctgc tcccaagcga ccccgtggag ggggcgcagg ggggagcagt gtagggacag
    841 ggggaggcgg cactggtggg gtgggtgggg gtgccggttc ggaagactct ggtgaccggg
    901 gagggactct gggaggggga acgctgggcc ccccaagccc tcctggggcc cccagccccc
    961 cagagccagg tggagaaatt gagctcgtgt tccggcccca ccccctgctc gtggagaagg
  1021 gagaatactg ccagacgagg tatgtgaaga caactgggaa tgccacagtg gaccacctct
  1081 ccaagtactt ggccctgcgc attgccctcg agcggaggca acagcaggaa gcaggggagc
  1141 caggagggcc tggagggggc gcctctgaca ccggaggacc tgatgggtgt ggcggggagg
  1201 gtgggggtgc cggaggaggt gacggtcctg aggagcctgc tttgcccagc ctggagggcg
  1261 tcagtgaaaa gcagtacacc atctacatcg cacctggagg cggggcgttc acgacgttga
  1321 atggctcgct gaccctggag ctggtgaatg agaaattctg gaaggtgtcc cggccactgg
  1381 agctgtgcta tgctcccacc aaggatccaa agtgacccca ccaggggaca gccagaggaa
  1441 ggggaccatg gggtatccct gtgtcctggt ctatcacccc agcttctttg tcccccagta
  1501 cccccagccc agccagccaa taagaggaca caaatgagga cacgtggctt ttatacaaag
  1561 tatctatatg agattcttct atattgtaca gagtggggca aaacacgccc ccatctgctg
  1621 ccttttctat tgccctgcaa cgtcccatct atacgaggtg ttggagaagg tgaagaaccc
  1681 tcccattcac gcccgcctac caacaacaaa cgtgcttttt tcctctttga aacctgcaaa
  1741 aaaa
RNF2 (accession No. NM_007212):
(SEQ ID NO: 122)
        1 gcgcctccgc ccctcgctcg ctcgctcctt cccgccctcc ccgcagcgcc ggccgagccg
      61 gcttcccctc agtctctcat gaatattgag cggcccctgt tgtatttccc gagctccatt
    121 gcggaagctg aggctcgcca tattgtgcgg cggcgccggc gtccgcggca gctgatacca
    181 gagtcttgct ccggccgcgg ccagcggagc cctgggctgg ggcaggagcc gcaatgtctc
    241 aggctgtgca gacaaacgga actcaaccat taagcaaaac atgggaactc agtttatatg
    301 agttacaacg aacacctcag gaggcaataa cagatggctt agaaattgtg gtttcacctc
    361 gaagtctaca cagtgaatta atgtgcccaa tttgtttgga tatgttgaag aacaccatga
    421 ctacaaagga gtgtttacat cgtttttgtg cagactgcat catcacagcc cttagaagtg
    481 gcaacaaaga atgtcctacc tgtcggaaaa aactagtttc caaaagatca ctaaggccag
    541 acccaaactt tgatgcactc atcagcaaaa tttatccaag tcgtgatgag tatgaagctc
    601 atcaagagag agtattagcc aggatcaaca agcacaataa tcagcaagca ctcagtcaca
    661 gcattgagga aggactgaag atacaggcca tgaacagact gcagcgaggc aagaaacaac
    721 agattgaaaa tggtagtgga gcagaagata atggtgacag ttcacactgc agtaatgcat
    781 ccacacatag caatcaggaa gcaggcccta gtaacaaacg gaccaaaaca tctgatgatt
    841 ctgggctaga gcttgataat aacaatgcag caatggcaat tgatccagta atggatggtg
    901 ctagtgaaat tgaattagta ttcaggcctc atcccacact tatggaaaaa gatgacagtg
    961 cacagacgag atacataaag acttctggta acgccactgt tgatcactta tccaagtatc
  1021 tggctgtgag gttagcttta gaagaacttc gaagcaaagg tgaatcaaac cagatgaacc
  1081 ttgatacagc cagtgagaag cagtatacca tttatatagc aacagccagt ggccagttca
  1141 ctgtattaaa tggctctttt tctttggaat tggtcagtga gaaatactgg aaagtgaaca
  1201 aacccatgga actttattac gcacctacaa aggagcacaa atgagccttt aaaaaccaat
  1261 tctgagactg aactttttta tagcctattt ctttaatatt aaagatgtac tggcattact
  1321 tttatggaca gatcttggat atgttgttca attttctttc tgagccagac tagtttacgc
  1381 tattcaaatc ttttcccctt tatttaagat ttcctttttg gaagggactg caattattca
  1441 gtattttttt ctttccttta aaaaaatata tctgaagttt cttgtgtttt tttttttccc
  1501 cacaaagtgt gtttccactt ggagcaccat tttgacccag gaatttttca tagtttctgt
  1561 attcttataa gattcagttg gctgtccttt tcctgctccc ctcaaaagat ttttagtcat
  1621 acagaatgtt aaatattatg tattctgact ttttttttcc cccggagtct tgtatattta
  1681 tagttttcta tataaactgt agtatcttca tgaagaccca aggctcaaat ttactgtcct
  1741 taaaaacaat tctcatagga ttattctttt catggtattt tcttccataa tatctcattt
  1801 taaaaagaag ttctttatga acttagtgtc cattgtcatg caatgttttt ttttttccat
  1861 tctttttccc tgtaattttg gaatttctgg tcctgggaag aatcaaacaa aatcttaagt
  1921 tctatgagaa cttggttcat tgacatattc tgctgaagaa agaaaaatta aattggtagt
  1981 aaaatatagt cttcaagtat acgtttgaga gtgctttttt ttgtattagt tctgctgtca
  2041 cttcatttcc tgtattatat gtgatgtttt tccccattaa aataccagag ataatggaga
  2101 tattttgcac tttagccttg atgaaaagta caagatatgt tcaaagcttc cctaattttt
  2161 ttcttatttg tagccacata agtttcaaga ataacatggc acacagaaca atggaaaaaa
  2221 gtttgtttcc attggaaaat tatatcattt tgggttgcca catcagttta taaatttggc
  2281 gctcttttaa ttacactctg tagaaggtta atagagcttg agccctgctt taatatgtag
  2341 tgaaagataa ttctgtagaa aaacgtcagc cagtagggta aagtcattct actgttctta
  2401 atttttatat tgaggaacaa tattgggtgt ttgggagcca gaaagctttg ttgacagatc
  2461 agaaataaga ttgacttggg tgttatattt catctctctc cagactctag gtatatttcc
  2521 aactttatat atcacagtat ttaaaaagac atgtttgcat tgagaaatta accctaaagg
  2581 gttttcaata gggtgtagac ctccagtacc tttgtaacta aagtctgtct agtcattgta
  2641 aatatttatc tgtcagtttt gacagattgg ggccagcttg atgttttaaa tcttcagccc
  2701 ggtatgaaaa cttaaaggta tatattcaat tttttaccat tttatggaaa atatttaaaa
  2761 tctgttttta cagggttttt tttttttttt tttttttgta atctgtgcca tgaaatttga
  2821 aaaccaccaa aaatcaaggg aacttttata tattcaattc cttttctggt gtaatgttaa
  2881 agttgtatag attattaatg catgcccact gaatataacc ctggttttgt gataaaactg
  2941 cttagatttt gttgatgaca ttagattagt agttgcatta aataactaaa ttcccattgt
  3001 gattaattga aattttgtct ttaagcagag agttatttgt gactataagc tttgtgctta
  3061 gagaatgtat gtgtttttat ctgtcagtat gggaggatat aaactgcatc attagtgaaa
  3121 ttattggttg tgtaatcctt tgtgaaatat aattctaggt atttgatagg gtattgagtg
  3181 tattttgtgt gtgtgtggat gtgtgttttg gggtacgggg agaggcgatg ctattggcca
  3241 tcactaccaa ccagggtttc aaaaagtatt acctaagtaa tttcttttat cactatctca
  3301 actgaggaag aaaaggctca ccacaagtgg tgtgaaggct ttgggtactt agttctaaat
  3361 ttttttatgg taacatatac atagccacat ttacagtttt aaccatttta aggcatgtaa
  3421 ttcagtgggg ttaggtacat tcacaatgtt gtgtaatgat caccgctgtc tacttgtaaa
  3481 actttttcat caccccaaac agaaactctg tgtgcaatta aagtaatgca tttctcttct
  3541 tcttaacccc t
PHF1 (accession No. NM_024165):
(SEQ ID NO: 123)
        1 ctccctcccc cccgccgcct cctcctcctg ccgctgccgc tgctttggct gctgcgtcat
      61 acgccccaga gccgccggga cggaggggct gggcctgggg accccccggc ctccgcctgc
    121 acgccccccc acgcccggac gtgccctctc cgcgcggggg actcgcctag gtctcctacg
    181 tctgcccctg cccggctccc ggcggcccca gctgtcaccg gcccccccag gatgcaatgg
    241 cgcagccccc ccggctgagc cgctctggtg cctcctcact ttgggaccca gcttctcctg
    301 ctcccacctc tggccccagg cctcggcttt gggagggtca agatgtgctg gccagatgga
    361 ctgatgggct gctatacttg ggtaccatca aaaaggtgga cagtgctagg gaggtgtgtc
    421 tggtccagtt tgaggatgat tcgcagtttc tggttctatg gaaagacatt agccctgctg
    481 ccctccctgg agaggaactc ctctgttgtg tctgtcgctc tgagactgtg gtccctggga
    541 accggctggt cagctgtgag aagtgtcgcc atgcttatca ccaggactgc catgttccca
    601 gggctccagc ccctggagag ggagagggca catcctgggt atgccgccag tgtgtctttg
    661 cgatcgccac caagagggga ggtgccctga agaagggccc ctatgcccgg gccatgctgg
    721 gtatgaagct ttctctgcca tatggactga aggggctgga ctgggatgct ggacatctga
    781 gcaaccgaca gcagagttac tgttactgtg gtggccctgg ggagtggaac ctgaaaatgc
    841 tgcagtgccg gagctgcctg cagtggttcc atgaggcctg cacccagtgt ctgagcaagc
    901 ccctcctcta tggggacagg ttctatgaat ttgaatgctg tgtgtgtcgc gggggccctg
    961 agaaagtccg gagactacag cttcgctggg tggatgtggc ccatcttgtc ctgtatcacc
  1021 tcagtgtttg ctgtaagaag aaatactttg attttgatcg tgagatcctc cccttcactt
  1081 ctgagaattg ggacagtttg ctcctggggg agctttcaga cacccccaaa ggagaacgtt
  1141 cttccaagct cctctctgct cttaacagcc acaaggaccg tttcatttca gggagagaga
  1201 ttaagaagag gaaatgtttg tttggtctcc atgctcggat gcctccccct gtggagcccc
  1261 ctactggaga tggagcactc accagcttcc cttcagggca gggccctggg ggaggggtct
  1321 cacgtcccct ggggaagcgc cggaggccgg agccagagcc cctgaggagg aggcagaagg
  1381 ggaaagtgga ggagctgggg ccaccctcag cagtgcgcaa tcagcccgag ccccaggagc
  1441 agagggagcg ggctcatctg cagagggcac tgcaggcctc agtgtctcca ccatccccca
  1501 gccctaacca gagttaccag ggcagcagcg gctacaactt ccggcccaca gatgcccgct
  1561 gcctgcccag cagccccatc cggatgtttg cttccttcca cccttctgcc agcaccgcag
  1621 ggacctctgg ggacagtgga cccccagaca ggtcacccct ggaacttcac attggtttcc
  1681 ccacagacat ccctaaaagt gccccccact cgatgactgc ctcatcttcc tcagtttcat
  1741 ccccatcccc aggtcttcct agacgctcag cacccccttc tcccctgtgc cgtagtttgt
  1801 ctcctgggac tgggggagga gtccgaggtg gggttggtta cctgtcccga ggggaccctg
  1861 tccgggtcct tgctcggaga gtacggcctg atggctctgt gcagtacctg gttgagtggg
  1921 gaggaggggg catcttctga acagcctgcc tctgcccagc tccccattca cacacaccgg
  1981 cactttcata ccctgacctc tgacctcacc tacagctggg atgtacctgg agagataggg
  2041 ggtagttctc cctactgccc aggctggaat ccaagagtgg ggagtgggga agaggccctc
  2101 ttctctaccc tccttcatga ttcctgaccc ctcccatcct tcccatttcc tttgatgtta
  2161 ttttgttaca gctttttaaa tattttttaa aattatttaa cccctggggg cagagactga
  2221 ggagggagga tgataaggga tcccggactc tgtatgattg aaataaagag aaataaacaa
  2281 atctagcagc tctgaaaaaa aaaaaaaaaa aa
MTF2 (accession No. NM_007358):
(SEQ ID NO: 124)
        1 gctgccattc ggcaccggag tcgctccgcg ctcccagaat gcaccggcag tccgcgggaa
      61 accaaaatgg cgaggggctg tattgaagtg ggctgtgttt gaggccggtg taagaacgct
    121 cattctaccc ccaacccttg tctccaagga cctcggtttg tgcgtgcata tgtgccgggt
    181 acccggtggg gcgggtgccc agtaagtgct cggactcgca ggggaagcgc ccacggggac
    241 ggattggttg ttttttcctg tatgaagcgg ttggcaccac tgaagtgacc gaatgagaga
    301 ctctacaggg gcaggtaatt cactggtcca caagcggtct cctttacgtc gaaaccaaaa
    361 gaccccaaca tccttgacca agctgtcttt acaggatgga cataaagcca aaaagccagc
    421 atgtaaattt gaagagggtc aggatgtcct agctagatgg tcagatggct tgttttatct
    481 tggcactatc aaaaagataa acatattgaa acagagctgc ttcatcatat ttgaagacag
    541 ttctaaatcc tgggttctct ggaaggacat tcaaacagga gccactggaa gtggggaaat
    601 ggtctgtaca atatgtcaag aagagtattc agaagctccc aatgaaatgg ttatatgtga
    661 caagtgtggc caaggatatc atcagttgtg tcacacacct catattgatt ccagtgtgat
    721 tgattcagat gaaaaatggc tctgtcggca gtgtgttttt gcaacaacaa caaagagggg
    781 tggtgcactt aagaaaggac caaatgccaa agcattgcaa gtcatgaagc agacattacc
    841 ctatagtgtg gcagaccttg aatgggatgc aggtcataaa accaatgtcc agcagtgtta
    901 ctgctattgt ggaggccctg gagactggta tttgaagatg ctacagtgct gcaaatgtaa
    961 gcagtggttt catgaggctt gtgtgcaatg ccttcaaaag ccaatgctat ttggagacag
  1021 attttatacg tttatatgct ctgtctgcag ttctggacca gaatacctca aacgtctacc
  1081 attacagtgg gtagatatag cacacctatg cctttacaac ctaagtgtta ttcataagaa
  1141 gaaatacttt gattctgaac ttgagcttat gacatacatt aatgaaaact gggatagatt
  1201 gcaccctgga gagctggcag acacaccaaa atctgaaaga tatgagcatg ttctggaggc
  1261 attaaatgat tacaagacca tgtttatgtc tgggaaagaa ataaagaaga agaagcattt
  1321 gtttgggttg cgaattcgtg ttcctcctgt gccaccaaat gtggctttca aagcagagaa
  1381 agaacctgaa ggaacatctc atgaatttaa aattaaaggc agaaaggcat ccaaacctat
  1441 atctgattca agggaagtaa gcaatggcat agaaaaaaaa ggaaagaaaa aatctgtagg
  1501 tcgtccacct ggcccatata caagaaaaat gattcaaaaa actgctgagc cacttttgga
  1561 taaggaatca atttcagaga atcctacttt ggatttacct tgttctatag ggagaactga
  1621 gggaactgca cattcatcca atacctcaga tgtggatttc acgggtgctt ccagtgcaaa
  1681 agaaactacc tcgtctagca tttccaggca ttatggatta tctgactcca gaaaaagaac
  1741 gcgtacagga agatcttggc ctgctgcaat accacatttg cggagaagaa gaggtcgtct
  1801 tccaagaaga gcactccaga ctcagaactc agaaattgta aaagatgatg aaggcaaaga
  1861 agattatcag tttgatgaac tcaacacaga gattctgaat aacttagcag atcaggagtt
  1921 acaactcaat catctaaaga actccattac cagttatttt ggtgctgcag gtagaatagc
  1981 atgtggcgaa aaataccgag ttttggcacg tcgggtgaca cttgatggaa aggtgcagta
  2041 tcttgtggaa tgggaaggag caactgcatc ctgactgtag gactgaacat tatgttcact
  2101 gcactctgat tttctgtagg tacagttcaa agccctaaag gagtctggct tttactatct
  2161 ttcttaaaaa aaaaaaaaag tcaaaaaaat tcaaaaaagg ggatgatact agccttaaca
  2221 tgtacctgtc aatgttatgg atattgtcat aaaaaggtat cttttaaaaa tcagaacaga
  2281 gacttaattt tttaaatctt aagatttgta gaatgtttct aggataggat attaaaaatg
  2341 attgaaaccc atgcatggtg ttagacaatt tttctaatta ttccattgag tcagtttttt
  2401 gtgattagtg attatcagag caaacatcat gtagatagca caagtatttg gagaaacgtt
  2461 gtttgttttg ttaccaaaat gttggaaaaa tttatttcaa taccttttag atttcataaa
  2521 gtgcagtgta tataatgcct actgaaagac tgtaaaatat tgaaattttc tttcaagcaa
  2581 agtgtaaaaa aatatattga gcctgtaaat tgctctgtga ctagacttca ttgtcgtctt
  2641 aatatattct tgcatgtgca tatatataca cacgtgtata tatatgtgtg tgattatgtg
  2701 acctatgcaa tacaaattat gggaatgggc agctttggag tatatatccc ataattcttt
  2761 tttcaggaat agttgcagta tttacacagc agcatttctt ctcaggcttt tattgggtgc
  2821 tgttgcttgc tatgtatgaa gagaaatgtg tcagacaagt ttagtgtgtt ctgaagaagg
  2881 gtgtgaacaa cagtgttcat gggcttttag aatgcttttc acttttagtc cttgtaactc
  2941 agctgttcag tacctaaaac aaattcaaat aatatgaaca ttatctccta ctagaagtaa
  3001 cgttttcaag ttttcatggc acattatgat tgtaaatgtc tctcattttt aacagtaagt
  3061 ctataggagt cccgtgaaga ttcctgaaat gtctgtagta actgttagtc atgtttgaat
  3121 aagtgtagta tgaacaaagt attttattgc acagggttaa caaacagtat gttgccagct
  3181 gaggctactg ctgttttatt acaacattac ctcttgtttt tataaagtgt accaagattt
  3241 aaattgataa ctttatttta cttgtaaaaa aaaagtttct tttatcacca gtgttacagt
  3301 tgtcttctgt ttctttttgt tttgttttat ttgttttcct ttttagccaa agagtgaaca
  3361 gaagattttc ttattttggt ggctattcat tttactttta aaagtgattg gtggatttta
  3421 gactaattat gggggaattt gccaccaaaa taaaaaatat gtaaagtgta gtgattacag
  3481 agtggttaaa atgtgggtta gtacttattt attccattaa ttgattattt gactgtttat
  3541 aaagaaagtt gctttatttc tttaaacatc ttcaaaagat gatcctttct tgtcacatta
  3601 tagccaaaag aagcagagaa cttcattgtc tgcatttggt tcctggttgg ccaggtataa
  3661 atgagcttta caaaagtgca aattaaaaac tgttacttct gtttacctcc accaaaactt
  3721 gattttcccc tagctattaa tttaaggttg cctttcctgc agctgcaata ttttgaataa
  3781 cacacagagt ttgtgttgat ttttgaatgt ttgtttatat ctaggggtaa tgaaaaatgt
  3841 aaatcccgtg tatccttatt cactccacct gtatcatatt atttcatttt ccccaaagtc
  3901 ctttaattct aactgaacac cagcagtatt tttagaaatt tttctttaac atacttggaa
  3961 gatgatttat ccagctgaac tgtctttaga cgtaattatt gtgaatgtct gttttatttt
  4021 ctcatggtgg ttcacatggc tctgatgttc agtttgtatt tttggaattg ctttacttag
  4081 aaattaaaac agaccaacat taaatgtgtg tattttttaa agagctaaaa aaaaaaaaaa
  4141 aaaa
PHF19 (accession No. NM_001286840):
(SEQ ID NO: 125)
        1 accagtaagt cgtgtattta gcattcattc atcaaagcct ccggatgcct cctacgtgcc
      61 ctgcactatg ctggtcttgg taatccgtgg tccctatccc agcgcccagt gtcaggggaa
    121 gctgatggag aatcgagctc tggatccagg gactcgggac tcctatggtg ccaccagcca
    181 cctccccaac aagggggccc tggcgaaggt caagaacaac ttcaaagact tgatgtccaa
    241 actgacggag ggccagtatg tgctgtgccg gtggacagat ggcctgtact acctcgggaa
    301 gatcaagagg gtcagcagct ctaagcaaag ctgcctcgtg actttcgaag ataattccaa
    361 atactgggtc ctatggaagg acatacagca tgccggtgtt ccaggagagg agcccaagtg
    421 caacatctgc ctagggaaga catcagggcc gctgaatgag atcctcatct gcgggaagtg
    481 tggcctgggt taccaccagc agtgccacat ccccatagcg ggcagtgctg accagcccct
    541 gctcacacct tggttctgcc gacgctgcat cttcgcactg gctgtgcgga aaggcggcgc
    601 gctgaagaag ggcgccatcg ccaggacgct gcaggccgtg aagatggtgc tgtcctacca
    661 gcccgaggag ctcgagtggg actcgcccca tcgcaccaac cagcagcaat gctactgcta
    721 ctgcggcggg cccggagaat ggtacctgcg gatgctgcaa tgttaccggt gcaggcagtg
    781 gttccacgag gcctgcaccc agtgcctcaa tgagcccatg atgtttggag accggtttta
    841 cctgttcttc tgctccgtgt gtaaccaggg cccagagtac atcgagaggc tgcccctgcg
    901 atgggtggat gtggttcacc tggccctcta taatctgggg gtacagagca agaagaagta
    961 ctttgacttt gaggagattc tggcctttgt caaccaccac tgggagctcc tgcagcttgg
  1021 caagctcacc agcaccccag tgacagatcg aggaccacat ctcctcaacg ctctgaacag
  1081 ttataaaagc cggttcctct gcggcaagga gatcaagaag aagaagtgca tcttccgcct
  1141 gcgcatccgc gtcccaccca acccgccagg gaagctgctg cctgacaaag gactgctgcc
  1201 aaatgagaac agcgcctcct ctgagctgcg taagagagga aagagcaagc ctggtttgtt
  1261 gcctcacgaa ttccagcagc agaaaaggcg agtttataga agaaaaagat caaagttttt
  1321 gctggaagat gctattccca gtagtgactt cacctcagcc tggagcacca accaccacct
  1381 ggctagcata tttgacttca cgctggatga aattcaaagt ttaaaaagtg ccagctcagg
  1441 ccagaccttc ttctcagatg tcgactccac cgacgctgcc agcacctctg gctctgcctc
  1501 caccagcctc tcctatgact ccagatggac agtgggcagc cgaaagagga agctggcagc
  1561 caaggcatac atgcccctgc gggcaaagcg gtgggcagct gagctggatg gacgctgccc
  1621 ctcggacagc agtgcagagg gggcttcagt ccccgagcgg ccagacgaag gcattgacag
  1681 ccacacattt gagagcatca gtgaagatga ctcatccctg tcccacctca agtcatctat
  1741 caccaactac tttggtgcag ctgggcggtt ggcctgtggg gagaagtacc aggtgttggc
  1801 tcggagggtc acacctgagg gcaaggttca gtacctggtg gagtgggaag ggaccacccc
  1861 ttactgacta gcccccgggg gtgccagggg tcctgaaaac caaaggagga gcagcagaag
  1921 ccataggctc cccagctttc tccaggctgg ggtgggagaa ggaagcagga cagagctgca
  1981 agtgcctggc agaatgccct gcctgcctgc ctgccaggcc aaggcctgcg tctctctgct
  2041 gtaccagctc tgttccaggg cctcctcagg ctcgttaccc ctgtgcctgt gtctctacac
  2101 actccacacc ccctcaaact ctgtttatct gttctctgac cttgtgtccc ctgcgctggg
  2161 acccttcctc ctgaggccca ggtctttgtc cccagttgtg tgccttgacc tctctcgccc
  2221 ctttctgggt gtgttcgcac atcctgtgtg tgcacagctg tccctccact ggatcccctt
  2281 cacacgtgac ccgtggggca gccagtcctc ccagggacta cataacaggc acctttgaga
  2341 gagcatggga gaaggtggat aagaggatgc tgctcagtgc ttttctcttc cactttcctg
  2401 ccactcccca ctaccctcgg agagaggtgg tgggatggga gagagcccct gtgaaagcct
  2461 gtgaggatct gaagagtaaa gggctgggtc tgcctcagaa ggcaccagca ccagggccca
  2521 ggtattaagg ctgagagtga aggctgccaa tgtcagcttt ggaggtccca gaagtcttct
  2581 gttctctggc ctcaccccct cagtcgccat agagctgggc ctggccttgc tggaatggag
  2641 gcatccttcc aaacctgggg gacgggggtg gggggtggta gtggtgggag ggaaaccatg
  2701 tcttgctaaa cctgtttctg gtgcctccca tccccagacc caccagacac cacacagcag
  2761 acaatacaca cccactcgca caagcttcca tccacatgtg ttgtactttc agctctaggc
  2821 atgcagacaa ccccacacgg ccacaccacc acatgcccaa gtgtacacac acagagccac
  2881 accgtccctc tgggcctgct ggctcctccc ttggctttcc cttggcccac ttccagggcc
  2941 caggtgctgc aactaaatgt gaaagctcag tggccgctcc ttctttcagc ccatcaacca
  3001 gcattggtcc catagggaag cacaggggac tcaccctctt tcatatccct tgccctgccc
  3061 tgaaatggac aatcactttt tgggataggt tgaaattttt aaagagcctg catcatttgg
  3121 ttccctcaaa gggaagccct tgccagtggg ggtttgaaag agaatttttg gaaccaacat
  3181 tcaaattctg cctcatctgg agggaaacca aaattgggag ggggaagagg acccctgatg
  3241 ttttgctgct tccagagata ttagaaactg actcacttga ttggaaaatg gacaaaagtg
  3301 ccttgacgtg gagggtgggc accagatggg gaccagcctt gccaactgct gctgtggcct
  3361 ccagcttggc tggttttgca ggccgccagc aggaaggcga aggtggtagt acagcaagag
  3421 gcactggcgg ggcagcaggc ctgcaggagc tgtttttcca ttgctaggcc tgacccctct
  3481 ctacctgtga gcgttcaggg ggtccctgag atagtttaga tgccccccca tcttagacct
  3541 cagctcccac agtgcctttt aagggggacc tcacctcctg tgcacagccc acccactttc
  3601 ctctgcttcc ctggcacagc ccaggcatag acgagctggc gttggaccca gttcttcccc
  3661 cttttcagcc ccacagctgc tgccacaggg gccaactagg gccaggtgga aggggagctg
  3721 agaagccaac ccctagccca ggggtgctgt gggaactggg atccaatttg tagcttcctg
  3781 cctggcttca gagagcccag caaccttcta ggcctgcttt ccagacttct gagatagcct
  3841 gggatgagca atcctgttat agtacatctg gaccttccct acctgggctc tggggaggct
  3901 gtgggcctgg agagggaaaa ggagggaggg ggtgtctgca ccacctggga agatagcaca
  3961 aggcctaatg aggtcaccct gactccccac cccagcattt cattcatacc agataatagc
  4021 tgcattactg ccaactgacc ttataaccct ctgcaccttc aaaaagattc atggttttta
  4081 attgctgctt ttaataacat ttgttaaagt tataattaat gtgtctgatt tatgatttaa
  4141 aacctccctt tgaacaatca aaaaaaaaaa aaaaaaaaaa a
SETDIA (accession No. XM_005255723):
(SEQ ID NO: 126)
        1 agtggtgttt ggtgcgcgcg ccggggaggt ggtggtgggg gggcgccgcc gccgccaccg
      61 ctgcggggcc gggtctcgcg ctgccgctgc cgccgcctcg cgccgctgag gtgccgcgcg
    121 aggtgggggg agggggagcc gctcgccggg agcggtgtaa atgagcaaag atggatcagg
    181 aaggtggggg agatgggcag aaggccccga gcttccagtg gcggaactac aagctcatcg
    241 tggatcctgc cttggaccct gccctgcgca ggccttctca gaaggtgtac cgctatgatg
    301 gagtccactt cagtgtcaac gactcaaagt atataccagt cgaagacctc caagaccccc
    361 gttgccatgt caggtccaaa aacagagact tttccctccc agtccctaag tttaagctgg
    421 acgagttcta tattggacag attccactga aggaagtgac ttttgcaagg ctgaatgaca
    481 acgtgcggga gaccttcctg aaggatatgt gccgtaagta cggtgaggtg gaagaggtag
    541 agatcctcct tcacccccgt acgcgcaagc acctgggcct ggcccgtgtg ctcttcacca
    601 gcactcgggg cgccaaggaa acggtcaaaa acctccacct tacctccgtc atgggcaaca
    661 tcatccatgc ccagcttgac atcaaaggac aacaacgaat gaaatactat gaactaattg
    721 tcaatggctc ctacacccct cagactgtgc ccactggggg caaggccctg agtgagaagt
    781 tccaaggctc gggtgcagcc actgagacgg ccgaatcccg ccgccgctct tcctctgaca
    841 cagctgccta cccagcaggc accactgcgg tgggcactcc tggcaacggc accccctgct
    901 cccaggacac aagcttctcc agcagccgac aagatacccc atcttccttt ggccagttca
    961 cacctcagtc ctcccaagga accccctaca cgtctcgggg cagcaccccc tactctcagg
  1021 actctgccta ctccagcagc accacttcaa cctccttcaa gccccggcgg tcagagaaca
  1081 gctaccaaga tgccttttcc cgccgccact tctctgcatc ttcagcctcc acaaccgcct
  1141 ccacggccat cgccgccacc actgcagcca ctgcctcatc ctccgcctct tcctcctcat
  1201 tgtcctcgtc ctcctcgtca tcctcttcct cctcgtcctc tcagtttcgt agttctgatg
  1261 caaactaccc agcgtattat gaaagctgga atcgctacca gcgccatact tcctacccac
  1321 cacgccgggc cacacgggag gaaccccctg gagccccttt tgctgaaaat acagctgagc
  1381 gcttcccacc ttcttacacc tcctacctgc cccccgagcc cagccggccc accgaccagg
  1441 actaccggcc tcctgcctca gaggctccac ccccggagcc tccagaacct ggtggaggcg
  1501 ggggtggagg agggcccagc cctgagagag aagaagttcg gacttccccc cgcccagcct
  1561 cccctgcccg ctctggctcc ccagccccgg agaccaccaa tgagagtgtg cccttcgccc
  1621 agcacagcag cctggattcc cgcatcgaga tgctgctgaa ggagcagcgc tccaagtttt
  1681 ccttcttggc ctctgacaca gaggaggagg aagagaacag cagcatggtc cttggggcca
  1741 gagatacagg gagtgaggtg ccttctgggt cagggcatgg gccctgcaca ccccctccgg
  1801 ccccagctaa ttttgaggat gtggcaccta cagggagcgg ggagccaggg gctacccggg
  1861 agtctcccaa ggcaaatgga cagaaccagg cttctccatg ctcttctgga gacgacatgg
  1921 agatctccga cgacgaccgg ggtggctcac cccctccggc cccgacgccc cctcagcagc
  1981 ctccgccacc tccccctccc ccgccgcctc ctcctcccta cctggcgtcc cttcctcttg
  2041 gttatcctcc ccaccaacct gcctacctcc tcccacccag acctgatggg ccgccgcccc
  2101 ctgagtaccc cccacctcct ccaccacccc cgcacatcta tgactttgtg aactccttgg
  2161 agctcatgga ccgacttggg gctcagtggg gagggatgcc catgtccttc cagatgcaga
  2221 cccagatgtt aactcggctc catcagctgc ggcagggcaa gggattgatt gccgcctcag
  2281 ctggcccccc cggtggggcc tttggggagg ccttcctccc gtttccaccc ccgcaggagg
  2341 cagcctacgg cttgccgtat gctctatatg cacaggggca ggagggcaga ggggcatact
  2401 cacgggaggc ctaccacctg cccatgccaa tggcagccga gcccctgccc tcctcctcag
  2461 tctcgggaga ggaggcccgg ctgccaccca gggaagaagc agagctggca gagggcaaga
  2521 ccctcccgac agcaggcacc gtgggccgtg tgctcgccat gctggtccag gagatgaaga
  2581 gcatcatgca gcgagacctc aaccgcaaga tggtggagaa cgtggccttc ggagcctttg
  2641 accagtggtg ggagagcaag gaggagaagg ccaagccatt ccagaacgcg gccaagcagc
  2701 aagccaagga ggaggataaa gagaagacga agctgaagga gcctggcctg ctgtccctcg
  2761 tggactgggc caagagcggg ggcactacgg gcatcgaggc tttcgccttt gggtcagggc
  2821 tgagaggggc cctgcggctg ccttcattca aggtaaagcg gaaagagcca tcggaaattt
  2881 ccgaggccag tgaggaaaag aggcctcgtc cctccactcc tgctgaggaa gatgaagacg
  2941 accctgaaca agagaaggag gctggagagc caggacgtcc ggggaccaag cccccgaagc
  3001 gggacgaaga gcgaggcaag acccagggca agcaccgcaa gtcctttgct ctggacagcg
  3061 aaggggagga ggcatcccag gagtcctcct cggagaagga tgaggaggat gacgaggaag
  3121 atgaggaaga tgaagatcga gaggaagctg tggataccac aaagaaggag acagaggtgt
  3181 cggatggcga ggacgaggaa agcgattcgt cttccaaatg ttctctgtat gctgactcag
  3241 atggcgaaaa tgacagcaca tcagactccg agagcagcag ctcttccagc tcctcatcct
  3301 cctcctcctc ctcgtcctca tcctcctcgt cctcttcatc ctctgagtcc tcctctgaag
  3361 atgaagagga agaggagcgg ccagcagccc ttccctcagc ctccccgccc cccagagaag
  3421 tcccagtgcc cacgccagca cctgtggagg tgccagtgcc ggaaagggtt gcaggctccc
  3481 cagtcacacc cctgcccgaa caggaggcgt ctccagcaag gcctgcaggc cccacggagg
  3541 agtcaccccc cagtgcgcct ctgcgtcccc cagaaccacc tgctgggccc ccggcccctg
  3601 ccccacgccc cgatgagcgt ccctcttctc ccatccccct cctgccccca cccaagaaac
  3661 gccggaaaac tgtctccttc tctgccatcg aggtggtgcc agccccggag ccccctccag
  3721 ccacaccgcc gcaggccaag tttcccggcc cagcctcccg caaggctccc cggggcgtgg
  3781 agcggaccat ccgcaacctg cccctggacc acgcatctct ggtcaagagt tggcccgagg
  3841 aggtgtcccg aggaggccgg agccgggctg gaggccgagg ccgcctcacc gaggaagagg
  3901 aggctgagcc agggacagag gtggacctgg cggtcctggc cgacctggcc ctgacccctg
  3961 cccggcgcgg gctgcctgcc ctgcctgctg ttgaagactc agaggccaca gagacatcgg
  4021 acgaggccga gcgccctagg cccctgctca gccacatcct cctggagcac aactatgccc
  4081 tggccgtcaa gcccacgccc cctgcgccag ccctgcggcc cccggagcca gtgcccgcac
  4141 ccgccgccct cttcagttcc ccagctgatg aggtcctgga ggcccccgag gtggtggtgg
  4201 ctgaggcgga ggagcccaag ccgcagcaac tgcagcagca gcgggaggag ggcgaagagg
  4261 agggggagga agagggggag gaagaggagg aggagtcctc tgacagcagc agcagcagcg
  4321 atggggaggg cgccctccgg aggcgcagcc tccgctccca cgcccggcgc cgccgccctc
  4381 cgcccccacc cccgccgcca ccgccccgcg cctacgagcc acgcagtgag tttgaacaga
  4441 tgaccatcct gtatgacatt tggaactcgg gcctggactc agaggacatg agttacctgc
  4501 ggcttacgta cgagcggctg ctgcagcaga caagcggggc tgactggctc aacgacactc
  4561 actgggtcca tcacacaatc accaacctga ccaccccaaa acgcaagcgg cggccccagg
  4621 atgggccccg ggagcaccag acaggctcag cccgcagcga aggctactac cccatcagca
  4681 agaaggagaa ggacaagtac ctggacgtgt gcccagtctc ggcccggcag ctggagggcg
  4741 tggacactca ggggacgaac cgcgtgctgt ccgagcgccg gtccgagcag cggcggctgc
  4801 tgagcgccat cggtacctcc gccatcatgg acagtgacct gctgaaactc aaccagctca
  4861 agttccggaa gaagaagctc cgatttggcc ggagccggat ccacgagtgg ggtctgtttg
  4921 ccatggaacc cattgctgct gacgagatgg tcatcgaata cgtgggtcag aacatccgtc
  4981 agatggtggc cgacatgcgg gagaagcgct acgtgcagga gggcattggc agcagctacc
  5041 tgttccgggt ggaccacgac accatcatcg atgccaccaa gtgtggcaac ctggccagat
  5101 tcatcaacca ctgctgcacg cctaactgct acgccaaggt catcaccatc gagtcccaga
  5161 agaagatcgt gatctactcc aagcagccca ttggcgtgga cgaggagatc acctacgact
  5221 acaagttccc actggaagac aacaagatcc cgtgtctgtg tggcacagag agctgccggg
  5281 gctccctaaa ctgaggtggg gcaggatggg tgcccacacc cctatttatt ccccctggtg
  5341 ccctgagctc ccagcacccc cccagcctta gtgggctcag cagggcccac atgcccccat
  5401 ctccaagcgt ggggttgggg gccccaagcc cagcgaggga gcctcagtcc ctggaggcag
  5461 cttctgcctc tcctgtcacc cctgcccacc accccctgat tgtttttctt tgcggagaag
  5521 aagctgtaaa tgttttgtag cagccagcag ctgtttcctg tggaaacctg gggtgccggc
  5581 ctgtacagat tctgtcctgg ggggctacac agtcctcttg ctttgtgtta atggggactt
  5641 ccccttacgc cctgcgtgta cccctcccca gtttaggggt ctctggggca gtggccatgt
  5701 tctccccctg ggggggctct gcacccccag tcctggggac tccgtgcctg gaaccctgcc
  5761 tcatctgttc ctgccagacc ctgagggtca cccttccacc ctggtgtcac tccccggctc
  5821 agccaggcca ggatggcggg gtgggtccct tttgctgggc tggactgtac atatgttaat
  5881 agcgcaaacc cgacgccaca tttttataat tgtgattaaa ctttattgta caaaa
SETD1B (accession No. NM_015048):
(SEQ ID NO: 127)
        1 aacggcatgg agaacagtca ccccccccac caccaccacc agcagccccc gccgcagccc
      61 ggcccttcgg gcgagaggag gaaccaccat tggagaagtt acaagttgat gattgacccg
    121 gctctgaaaa aggggcatca taaactgtac cgctacgatg ggcagcattt cagcctggcg
    181 atgtccagca accgcccggt ggaaattgtc gaagatcccc gggtcgtcgg gatctggacc
    241 aaaaacaagg agctggagct gtcggtgccc aaattcaaga tcgatgagtt ctacgtgggc
    301 ccggtgcctc cgaagcaggt gacatttgcc aagctgaatg ataacatccg tgaaaacttc
    361 ctgagggaca tgtgcaagaa gtatggggag gtggaggagg tggagatttt gtacaacccc
    421 aagaccaaga agcacctggg catcgccaag gtggtctttg ccacggtccg gggagccaag
    481 gatgccgttc agcacttgca cagcacttcc gtcatgggca acattatcca cgtggagctg
    541 gacaccaaag gggaaacccg aatgcggttc tatgaactgt tggtcactgg ccgatacacc
    601 ccccagaccc tcccagtggg cgagctggac gctgtctctc caatcgtgaa tgagaccctg
    661 cagctgtcag atgccctgaa gcgcctcaag gatggaggcc tgtctgcagg ctgtggctcc
    721 ggctcctcct ctgtcacccc caatagcggt gggacaccct tctcccagga cacagcttat
    781 tccagctgcc gcctggacac acccaactcc tatggacagg gcaccccgct cacaccgcgc
    841 ctgggcaccc ctttctcaca ggactccagc tactccagcc gccagcccac accctcatac
    901 ctcttcagcc aggaccctgc agtgaccttc aaggcccggc gccacgagag caagttcacg
    961 gacgcctaca accgccgcca cgaacatcat tatgtacaca attctcccgc ggtcactgcg
  1021 gtggccgggg ccacagccgc tttccggggt tcctcggacc tcccgttcgg agcagtcggc
  1081 ggcactgggg gcagcagcgg tcccccgttc aaggctcaac cacaggattc agccacattt
  1141 gcccacactc caccacccgc ccaagcaacc cctgctcctg gattcaagtc tgctttctct
  1201 ccgtatcaga ccccagtggc ccacttccct ccacccccgg aagagcccac cgccacagcc
  1261 gcttttgggg cccgcgacag tggggagttc cggagggcac cggcgccccc acccctgcca
  1321 cctgctgagc ctctggccaa ggagaagcca ggcacgccac ccggcccgcc gccccccgac
  1381 accaacagca tggagctggg cggccggccc accttcggct ggagtcctga gccctgtgac
  1441 agccctggca cgcccacgct ggagtcgtcc cctgcagggc cagagaaacc ccacgacagc
  1501 ctggactcgc gcatcgagat gctgctgaag gagcagcgca ccaagctgct cttcctgagg
  1561 gagccggact cggacaccga gctgcagatg gagggcagcc ccatctcctc ctcctcctcc
  1621 cagctctccc cactggcccc ctttggcacc aactcccagc caggcttccg gggccccacg
  1681 cccccctcgt cacgcccctc cagcaccggc ctggaggata tcagcccaac acccctccca
  1741 gactccgacg aggacgagga gctcgacctg ggccttgggc ctcggcctcc acctgagcca
  1801 ggccccccgg accctgctgg gcttctgagc cagacagctg aggtggcctt ggacctggtt
  1861 ggagacagaa ccccgacctc agagaagatg gatgagggcc agcagtcctc aggcgaggac
  1921 atggagatct cggatgacga gatgccctcg gcccccatca ccagcgctga ctgccccaag
  1981 cccatggtgg tgaccccagg agcggcagcc gtggcagccc cttctgtgct agccccaacc
  2041 ctgccgctgc ccccgccacc tggcttcccc ccgctgcccc ccccaccacc accaccccca
  2101 ccgcagcctg gcttccccat gcccccaccg ctgcccccac cgccgccccc accccctcca
  2161 gcccaccctg ctgtgacagt gcccccacca cccttgccag cgccgcctgg agtcccgccc
  2221 ccacccatcc tgccaccact gccccccttt ccgccgggcc tgttccctgt gatgcaggtg
  2281 gacatgagcc acgtgctggg tggccagtgg ggcggcatgc ccatgtcctt ccagatgcaa
  2341 acgcaggtgc tcagccggct gatgacgggc cagggcgcct gcccctaccc gcccttcatg
  2401 gccgctgcgg ccgccgctgc ctcagctggg ctccagtttg tcaacctgcc gccctaccgg
  2461 ggccccttct ccctgagcaa ctccggccca ggccgcgggc agcactggcc accactgccc
  2521 aagtttgacc cgtcagtgcc tccaccaggc tacatgccac gccaggagga cccacacaaa
  2581 gccacggtgg atggcgtcct gctggtggtc ctcaaagaac tcaaggccat catgaagcgt
  2641 gacctgaacc gcaagatggt ggaagtggtg gctttccggg cctttgacga gtggtgggac
  2701 aagaaggagc ggatggccaa ggcctcgctg accccggtga agtcgggcga gcacaaggac
  2761 gaggacaggc cgaagcccaa ggaccgcatc gcctcgtgcc tgctggagtc atggggcaag
  2821 ggcgagggcc tgggctacga gggcctgggc ctgggcattg ggctgcgtgg ggccattcgc
  2881 ctgccctcct tcaaggtcaa gaggaaggag ccaccagaca ccacctcatc tggcgaccag
  2941 aagcggctgc ggccctcgac ctctgtggat gaggaagatg aagagtccga gcgagagcga
  3001 gaccgggata tggcagacac cccctgtgag ctcgccaagc gggaccccaa gggcgtgggt
  3061 gtgcggcggc ggccggcgcg gcctctggag ctggacagtg gtggggagga ggacgagaag
  3121 gagtcattgt cggaggaaca ggagagcacc gaggaggaag aggaggcgga ggaggaggag
  3181 gaggaggaag atgacgacga tgacgacagt gatgaccggg acgagtctga gaacgatgac
  3241 gaggacacag ccctgtcaga ggcgagtgag aaggacgaag gggactcgga tgaagaggag
  3301 acagtgagca ttgtaacctc caaggccgaa gccacgtcgt ccagtgagag ttccgagtct
  3361 tctgagtttg agtcaagctc cgagtcctcg ccctcatcct cggaggatga ggaggaggta
  3421 gtggccaggg aagaggagga agaagaggag gaggaggaga tggtggccga ggaaagcatg
  3481 gcttctgcag gccctgagga ctttgagcag gacggggagg aagcggctct ggccccgggg
  3541 gcacctgcag tggactcgtt gggcatggaa gaggaggtgg acatcgagac tgaggctgtg
  3601 gcccctgagg agcggccctc catgctggac gagcccccct tgcctgtggg tgttgaagag
  3661 ccagcggact ccagggagcc gcctgaggaa ccaggcctga gccaggaagg ggccatgttg
  3721 ctgtctccag agccccctgc caaggaggtg gaggctcgac ccccattgtc ccctgagcga
  3781 gctccagaac atgacctgga agtggagccg gagcccccta tgatgctccc cttgccgctg
  3841 caaccaccat tgccgccccc acgaccaccc cggccaccca gcccaccgcc ggagcctgag
  3901 accacagatg cctcacaccc atctgtccct ccggagcccc ttgccgagga ccaccccccg
  3961 catactccag gcctctgtgg cagcctggcc aagtcgcaga gcacagagac ggtgccagcc
  4021 acaccaggcg gggagccccc gctatcaggg ggcagcagtg gcctgtccct gagctctccg
  4081 caagtgcccg gcagcccctt ctcctaccca gccccgtccc ctagcttgag cagtgggggc
  4141 ctccctcgga cacctggccg ggacttcagc ttcacaccca ccttctccga gcccagcggg
  4201 cccttgctcc tgcccgtctg cccactcccc actggccgac gcgatgaacg ctccgggccc
  4261 ctggcctccc cggtgctcct ggagacgggc ctgcccctcc ctctgcccct tcccctgccc
  4321 ttgcccttgg cattgcccgc cgtcttgcgg gcccaggctc gtgcgcccac cccgctgcca
  4381 cccctgctgc ccgcccccct ggcctcttgc cctcccccaa tgaagaggaa gccgggccgg
  4441 ccccggcgat ccccaccatc tatgctctcc ttggatgggc ccttggtccg accaccagca
  4501 ggggccgccc ttggaaggga actcctgctc ctgccgggcc agccacagac ccccgtcttc
  4561 cccagcaccc atgacccccg gacggtgacc ctggacttcc ggaacgcggg gatcccagcc
  4621 cctccaccac cccttccccc ccagccaccc ccacccccac ctcccccacc tgtagagccc
  4681 accaagctgc cctttaagga gctagacaac cagtggccct ccgaggccat tcctccgggc
  4741 ccccgtgggc gcgatgaggt cactgaggaa tacatggagt tggccaagag ccgggggccg
  4801 tggcgccggc cacctaagaa gcgccatgag gacctggtgc cacctgcggg ctcgcccgaa
  4861 ctctcgccac cccagcccct cttccggccc cgctcggagt ttgaggagat gaccatcctg
  4921 tatgacatct ggaacggtgg catcgatgag gaggacatcc gcttcctgtg tgtcacctac
  4981 gagcgactgc tacagcagga caatggcatg gactggctta acgacacgct ctgggtctac
  5041 catccctcca ccagcctctc ttcagctaag aagaagaaac gggacgatgg catccgcgag
  5101 cacgtgacgg gctgtgcccg cagtgagggc ttctacacca tcgacaagaa ggacaagctc
  5161 agatacctca acagcagccg tgccagcacc gatgagcccc ccgcagacac ccagggcatg
  5221 agcatcccag cacagcccca cgcctccacc cgggcaggct cggagcggcg ttcggagcag
  5281 cgccgcctgc tgtcctcctt cactggcagc tgtgacagtg acctgctcaa gttcaaccag
  5341 ctcaagttcc ggaagaaaaa gctcaagttc tgcaagagcc acattcacga ctggggcttg
  5401 ttcgccatgg agcccatcgc ggctgacgag atggtcatcg agtacgtggg ccagaatatc
  5461 cgtcaggtga tcgcagacat gcgggagaag cgttatgagg acgagggcat cgggagcagc
  5521 tacatgttcc gggtggacca tgacaccatc atcgacgcca ccaagtgcgg caacttcgcg
  5581 cgcttcatca accacagctg caaccccaac tgctatgcca aggtgatcac ggtggagtca
  5641 cagaagaaga tagtcatcta ctcgaagcag cacattaacg tcaatgagga gattacctat
  5701 gactataagt tccccatcga ggacgtcaag atcccctgcc tctgtggctc cgagaactgc
  5761 cgggggaccc tcaactaggc cccggcacca gactcaaagg atgtcagccg tagccctggg
  5821 actcccgagc gtggagcccc tggccccggg gcccggcccc ccgcgcccgc ccccatttca
  5881 ggtgctgtcc tctacccagc ggccattcag ggcctggcgc cccacactac cccctggagc
  5941 ccctggctcc ggcccctccg cgggaaaggg cttctctgtc gttcagccca cgtctctctc
  6001 attttaacaa acgccccttt caggatttct gtttaactcc agcatcagct tctctctctc
  6061 cgtctctcct cccctctctc tcttctctgt ctcttctctc tcccaccatc accctcggcc
  6121 tcttcctgtg aatgctgcta cgttgttttg tcttctctat ttttttcctc gttgtgagaa
  6181 aagacattta accgttgaaa tgtgaaggtg gaatcagaga ggggccccgc gggggtctgc
  6241 agaggcctca gtgtggctgt gcgtggcccg tgtcctggaa gccacccgga cctggacgca
  6301 gggccaggtg ctgtgggaag gatggaggcc cccacggcct tgacctcaga acactacgcc
  6361 ctgaaagcgc ccctcactgc ccgtgggcac agtgaggaga ccccacacct ttccccaccc
  6421 gagctgcagc ctgttccttc cccagaggcc tggggcacca ctgacccggt ggaccctgat
  6481 ggagctaagc tgtcccaggc aggggtctcc gctctgggct ttccctgcca cctcacaccc
  6541 cagcaccccc taaaccttgg gttcaatgtt tactttctca ttcggatgcc agcaacgcgg
  6601 gagcctctcg gaggccccag tgcaggtgag gggcgctgag aacgcgggca gccactctct
  6661 tctgcccttg ccttcgccct gggtgggaca gggctcccaa gggcaggcgg gtcccccagt
  6721 cccgccatta cgggttgtca gaccgtctgc gtgtggcatt ttttggctta taagcttcac
  6781 ccactcaccc ccaacccaca ccccacatcc ccctgccggc agcccctcaa cctaagaagg
  6841 ccagagcata tttattttcg gagggagcag attacttctc ccagagaaag gaaaatcttg
  6901 gaaaagattt aaaaacacaa atctaagcct tgacggtttt tttttccctt ttgaccccct
  6961 tcccatctct tcagaattta ttcccatggc tttttttttt cttgtgcgtg tataaaatca
  7021 aaaggaaggg gaaaaaggtt tttgaagttc agaaccaact tctgtatata gaggctgccg
  7081 caaaggactt tctcttggga acattgtttc ttgtagaaac atgcgggaag acattttttg
  7141 ctcatttctt tgtacttcca aaaaaaaagg aaaaaaaaga caaaagcaag tccccccgta
  7201 ccccagaaag cagaggaggc gtgtaaataa tttctggaaa gtgactgttg tgacccggag
  7261 tcctcatcaa gatgagcgcg ctccatgagg gagctgctcc caccctgcgg acgcaggcgg
  7321 ccggagcctc tggtatctca gcttgtgtca agcttgttat catgtaaatt ctgtacaaag
  7381 aattgttatt tttctctttt ttgttgttgg tggttttgtt gtgtgttttt tgttgttttt
  7441 tttttattcc tttcccccag gccctctcta tttgagactg tgcccgccgg tttcaagatc
  7501 aaggaaattg gtggcaacaa gacacagatg gggtacctgg gcacagcggc gaacttctct
  7561 tccgtttgcg gttttctgcc taattgtgca actgaggaaa taatttattt ttcacatgag
  7621 gaaatgcgta gcttgtagag acggctgatt caagttacat gtacagcctc caaagggctg
  7681 tctccattct gtccccttcc cataaaagaa gtgggggtgt tcgagaagac cagggaaggg
  7741 acccttgcct cacccctccc cctggcctca ccttgctccc agccatcgtg cccagtgtta
  7801 acctcggctg gccttcacta aggggactag acctccctct ccccaggagc cccagcccca
  7861 gagtggtttg caataatcaa gatatgtgtc gagtcatttt tctttcaact ccctcatttt
  7921 tcattgaaca aatctctgct tttcaagagt tgggggtttc tgctattttt tgctttctct
  7981 ccctccccct gcaaagatga gaaccaatga gttttaggga tgtttgtgcg ggtagactcc
  8041 atcatccata tgtaacttgt tttgaagaga agtgtttccg ttgtgtgtct tgatgtaaat
  8101 atttgttcat atttttgtga attcaatact atgtaccatt gtattatagt aacttttata
  8161 aagcaaacca taaatatact gacttttctt acaga
CXXC1 (accession No. NM_001101654):
(SEQ ID NO: 128)
        1 ggaaagagtg gtggcaggtg aagtcggaga cgacagagga actggtttcc tccgccccgc
      61 aaggcacaca gcctgccgac gccccattaa tacatgtgga aggggaaaga gactgaatgg
    121 aggaatgaat acaacttgat ccaggtcgtg cttcggaagc ggtcacttta cctgtgaacc
    181 tctctgcctg acaaacgggc aatgtacgga atcaaccacc aagatggcgg cgcccgtgaa
    241 gaatccgcaa ttaggtcgcc gtcatatgtc gcctaggaac gtacggaatt cgacccacgt
    301 acggaatcgg attccaagat gacggcatct atgaggaagt cacgcagtag gtgcagccat
    361 gttgcctgta cgtcgaggcc gtacaagcag ccgccgtacg gactctactg acaaggtggc
    421 ggcgccctcg ggaaagccac attagagcgc ggccatgttc ccggcgaaca tatggattcg
    481 gccaccatac ggatacgata agcaagatgg cggcgcctga ggggtcttgg gggctctagg
    541 ccggccacct actggtttgc agcggagacg acgcatgggg cctgcgcaat aggagtacgc
    601 tgcctgggag gcgtgactag aagcggaagt agttgtgggc gcctttgcaa ccgcctggga
    661 cgccgccgag tggtctgtgc aggttcgcgg gtcgctggcg ggggtcgtga gggagtgcgc
    721 cgggagcgga gatatggagg gagatggttc agacccagag cctccagatg ccggggagga
    781 cagcaagtcc gagaatgggg agaatgcgcc catctactgc atctgccgca aaccggacat
    841 caactgcttc atgatcgggt gtgacaactg caatgagtgg ttccatgggg actgcatccg
    901 gatcactgag aagatggcca aggccatccg ggagtggtac tgtcgggagt gcagagagaa
    961 agaccccaag ctagagattc gctatcggca caagaagtca cgggagcggg atggcaatga
  1021 gcgggacagc agtgagcccc gggatgaggg tggagggcgc aagaggcctg tccctgatcc
  1081 agacctgcag cgccgggcag ggtcagggac aggggttggg gccatgcttg ctcggggctc
  1141 tgcttcgccc cacaaatcct ctccgcagcc cttggtggcc acacccagcc agcatcacca
  1201 gcagcagcag cagcagatca aacggtcagc ccgcatgtgt ggtgagtgtg aggcatgtcg
  1261 gcgcactgag gactgtggtc actgtgattt ctgtcgggac atgaagaagt tcgggggccc
  1321 caacaagatc cggcagaagt gccggctgcg ccagtgccag ctgcgggccc gggaatcgta
  1381 caagtacttc ccttcctcgc tctcaccagt gacgccctca gagtccctgc caaggccccg
  1441 ccggccactg cccacccaac agcagccaca gccatcacag aagttagggc gcatccgtga
  1501 agatgagggg gcagtggcgt catcaacagt caaggagcct cctgaggcta cagccacacc
  1561 tgagccactc tcagatgagg acctacctct ggatcctgac ctgtatcagg acttctgtgc
  1621 aggggccttt gatgaccatg gcctgccctg gatgagcgac acagaagagt ccccattcct
  1681 ggaccccgcg ctgcggaaga gggcagtgaa agtgaagcat gtgaagcgtc gggagaagaa
  1741 gtctgagaag aaggtgatgg agaggaagga ggagcgatac aagcggcatc ggcagaagca
  1801 gaagcacaag gataaatgga aacacccaga gagggctgat gccaaggacc ctgcgtcact
  1861 gccccagtgc ctggggcccg gctgtgtgcg ccccgcccag cccagctcca agtattgctc
  1921 agatgactgt ggcatgaagc tggcagccaa ccgcatctac gagatcctcc cccagcgcat
  1981 ccagcagtgg cagcagagcc cttgcattgc tgaagagcac ggcaagaagc tgctcgaacg
  2041 cattcgccga gagcagcaga gtgcccgcac tcgccttcag gaaatggaac gccgattcca
  2101 tgagcttgag gccatcattc tacgtgccaa gcagcaggct gtgcgcgagg atgaggagag
  2161 caacgagggt gacagtgatg acacagacct gcagatcttc tgtgtttcct gtgggcaccc
  2221 catcaaccca cgtgttgcct tgcgccacat ggagcgctgc tacgccaagt atgagagcca
  2281 gacgtccttt gggtccatgt accccacacg cattgaaggg gccacacgac tcttctgtga
  2341 tgtgtataat cctcagagca aaacatactg taagcggctc caggtgctgt gccccgagca
  2401 ctcacgggac cccaaagtgc cagctgacga ggtatgcggg tgcccccttg tacgtgatgt
  2461 ctttgagctc acgggtgact tctgccgcct gcccaagcgc cagtgcaatc gccattactg
  2521 ctgggagaag ctgcggcgtg cggaagtgga cttggagcgc gtgcgtgtgt ggtacaagct
  2581 ggacgagctg tttgagcagg agcgcaatgt gcgcacagcc atgacaaacc gcgcgggatt
  2641 gctggccctg atgctgcacc agacgatcca gcacgatccc ctcactaccg acctgcgctc
  2701 cagtgccgac cgctgagcct cctggcccgg accccttaca ccctgcattc cagatggggg
  2761 agccgcccgg tgcccgtgtg tccgttcctc cactcatctg tttctccggt tctccctgtg
  2821 cccatccacc ggttgaccgc ccatctgcct ttatcagagg gactgtcccc gtcgacatgt
  2881 tcagtgcctg gtggggctgc ggagtccact catccttgcc tcctctccct gggttttgtt
  2941 aataaaattt tgaagaaacc aaggaaaaaa aaaaaa
ASH2L (accession No. NM_004674):
(SEQ ID NO: 129)
        1 cacagcaacg cgcgcgagag aagagagtat tctcgcgaga agtccagggg tggccgtgat
      61 ggcggcggca ggagcaggac ctggccagga agcgggtgcc gggcctggcc caggagcggt
    121 cgcaaatgca acaggggcag aagaggggga gatgaagccg gtggcagcgg gagcagccgc
    181 tcctcctgga gaggggatct ctgctgctcc gacagttgag cccagttccg gggaggctga
    241 aggcggggag gcaaacttgg tcgatgtaag cggtggcttg gagacagaat catctaatgg
    301 aaaagataca ctagaaggtg ctggggatac atcagaggtg atggatactc aggcgggctc
    361 cgtggatgaa gagaatggcc gacagttggg tgaggtagag ctgcaatgtg ggatttgtac
    421 aaaatggttc acggctgaca catttggcat agatacctca tcctgtctac ctttcatgac
    481 caactacagt tttcattgca acgtctgcca tcacagtggg aatacctatt tcctccggaa
    541 gcaagcaaac ttgaaggaaa tgtgccttag tgctttggcc aacctgacat ggcagtcccg
    601 aacacaggat gaacatccga agacaatgtt ctccaaagat aaggatatta taccatttat
    661 tgataaatac tgggagtgca tgacaaccag acagagacct gggaaaatga cttggccaaa
    721 taacattgtt aaaacaatga gtaaagaaag agatgtattc ttggtaaagg aacacccaga
    781 tccaggcagt aaagatccag aagaagatta ccccaaattt ggacttttgg atcaggacct
    841 tagtaacatt ggtcctgctt atgacaacca aaaacagagc agtgctgtgt ctactagtgg
    901 gaatttaaat gggggaattg cagcaggaag cagcggaaaa ggacgaggag ccaagcgcaa
    961 acagcaggat ggagggacca cagggaccac caagaaggcc cggagtgacc ctttgttttc
  1021 tgctcagcgc cttccccctc atggctaccc attggaacac ccgtttaaca aagatggcta
  1081 tcggtatatt ctagctgagc ctgatccgca cgcccctgac cccgagaagc tggaacttga
  1141 ctgctgggca ggaaaaccta ttcctggaga cctctacaga gcctgcttgt atgaacgggt
  1201 tttgttagcc ctacatgatc gagctcccca gttaaagatc tcagatgacc ggctgactgt
  1261 ggttggagag aagggctact ctatggtgag ggcctctcat ggagtacgga aaggtgcctg
  1321 gtattttgaa atcactgtgg atgagatgcc accagatacc gctgccagac tgggttggtc
  1381 ccagccccta ggaaaccttc aagctccttt aggttatgat aaatttagct attcttggcg
  1441 gagcaaaaag ggaaccaagt tccaccagtc cattggcaaa cactactctt ctggctatgg
  1501 acagggagac gtcctgggat tttatattaa tcttcctgaa gacacagaga cagccaagtc
  1561 attgccagac acatacaaag ataaggcttt gataaaattc aagagttatt tgtattttga
  1621 ggaaaaagac tttgtggata aagcagagaa gagcctgaag cagactcccc atagtgagat
  1681 aatattttat aaaaatggtg tcaatcaagg tgtggcttac aaagatattt ttgagggggt
  1741 ttacttccca gccatctcac tgtacaagag ctgcacggtt tccattaact ttggaccatg
  1801 cttcaagtat cctccgaagg atctcactta ccgccctatg agtgacatgg gctggggcgc
  1861 cgtggtagag cacaccctgg ctgacgtctt gtatcacgtg gagacagaag tggatgggag
  1921 gcgcagtccc ccatgggaac cctgaccagg tccctctttt ctgtcaagga ctttctggga
  1981 ataatactgg gggttttgtt tttgtttttg aactgtctca aatgttctcc caaagatgct
  2041 aaaaacacag cctctccttt tagcaagtta aaaggctggg taggactgcg ggagactgcc
  2101 tgcctttcac cattttctcc ccacttccag tgactgctct tattttgtgt accataagcc
  2161 aacaaccgct gactccagga ttgcataagc cccctgtgaa atcggtgctg tactgcatac
  2221 cctgccagct gtgacttgtt atcctactat attttctaag gagtgaataa tattgtccga
  2281 gtaactaact tatttaaaag acatttcctt ctgtgggcat tgactgtatc ccacctgttt
  2341 tccaaggaaa tggtaacctg tttctgagaa cacctgaaat caatggctat acattccaaa
  2401 ccaatctaaa cgctatttcc ttttggtgtg ggtttggttt tgttcatttt gaaatacact
  2461 tttgaacact gagatccgta aaactactag atctctggaa gtgtaattgt gaaagaaact
  2521 tgcttgcagc tttaacaaaa tgagaaactt cccaaataaa acttgttttg aagtttatgt
  2581 gacactttgc ttcccttcag attgggtgcc tcttggtgac agtgttcaga aatgtaagca
  2641 gcacgaggaa gggagctggc actgggagga agagccgggt ttctgagttg tgttttggct
  2701 gctttcctat tgctcccatt cttgccaatc agccaccccc tttcctgtga aaatctgcca
  2761 ccttgaggag aggaacaaga gtttaaaagg gctaatgatc tccctcccgg tcttcccttg
  2821 gaacatggat gttgatatat gtgcgggtgg tttcctgtct tgcttatctt cctttgccct
  2881 gagctgatgg ctaaagggca gttttcggac tattaaagac tgaaatgtaa gaatgagcct
  2941 tctaggctgg gcgc
DPY30 (accession No. NM_032574):
(SEQ ID NO: 130)
        1 tggcgcggtg cagggctctt aagaacgaac ggcttgggcg cgggtaatca gctccctttc
      61 ccccactttc tcacttattc taggtacttg ggactgtcgt agagtttcca gaccccatgt
    121 aggcgcccag tcgtggactg tcccactctg ctgctctact gctcgtggtg ctcccgcgcc
    181 cagactggta tccggggact gtgacttgca gggtccgcca tggagccaga gcagatgctg
    241 gagggacaaa cgcaggttgc agaaaatcct cactctgagt acggtctcac agacaacgtt
    301 gagagaatag tagaaaatga gaagattaat gcagaaaagt catcaaagca gaaggtagat
    361 ctccagtctt tgccaactcg tgcctacctg gatcagacag ttgtgcctat cttattacag
    421 ggacttgctg tgcttgcaaa ggaaagacca ccaaatccca ttgaatttct agcatcttat
    481 cttttaaaaa acaaggcaca gtttgaagat cgaaactgac ttaatgggaa gaacagaaaa
    541 atttagttgc tactgtagat ttacatgatt aagaggcagc tttaattgcc atgatcattc
    601 cctctttttg gatgtataag aaccttccgg acaacagaac ctatttctgg aattgcagaa
    661 gataacatat ttcccttatt ttgatttaat caccataaac catacctatt taatgagtgt
    721 attctgtgca atttttttct cagattgtct ttaactttgt ttttaaaatg accttcaaaa
    781 taaactgtca aaacaccatt
RBBP5 (accession No. NM_005057):
(SEQ ID NO: 131)
        1 ggaagccgcg gggccttcta aggccgaaag tcttcggagc ttgcgccagt ctcttcgcgg
      61 cgtccaccac ttagacgcaa gttgctgaag ccggccgggg agaaggtgtt gttgccggag
    121 ctgagaccgg gcggccacag tccgcaggga tgaacctcga gttgctggag tcctttgggc
    181 agaactatcc agaggaagct gatggaactt tggattgtat cagcatggct ttgacttgca
    241 cctttaacag gtggggcaca ctgcttgcag ttggctgtaa tgatggccga attgtcatct
    301 gggatttctt gacaagaggc attgctaaaa taattagtgc acacatccat ccagtgtgtt
    361 ctttatgctg gagccgagat ggtcataaac tcgtgagtgc ttccactgat aacatagtgt
    421 cacagtggga tgttctttca ggcgactgtg accagaggtt tcgattccct tcacccatct
    481 taaaagtcca atatcatcca cgagatcaga acaaggttct cgtgtgtccc atgaaatctg
    541 ctcctgtcat gttgaccctt tcagattcca aacatgttgt tctgccggtg gacgatgact
    601 ccgatttgaa cgttgtggca tcttttgata ggcgagggga atatatttat acgggaaacg
    661 caaaaggcaa gattttggtc ctaaaaacag attctcagga tcttgttgct tccttcagag
    721 tgacaactgg aacaagcaat accacagcca ttaagtcaat tgagtttgcc cggaagggga
    781 gttgcttttt aattaacacg gcagatcgaa taatcagagt ttatgatggc agagaaatct
    841 taacatgtgg aagagatgga gagcctgaac ctatgcagaa attgcaggat ttggtgaata
    901 ggaccccatg gaagaaatgt tgtttctctg gggatgggga atacatcgtg gcaggttctg
    961 cccggcagca tgccctgtac atctgggaga agagcattgg caacctggtg aagattctcc
  1021 atgggacgag aggagaactc ctcttggatg tagcttggca tcctgttcga cccatcatag
  1081 catccatttc cagtggagtg gtatctatct gggcacagaa tcaagtagaa aactggagtg
  1141 catttgcacc agacttcaaa gaattggatg aaaatgtaga atacgaagaa agggaatcag
  1201 agtttgatat tgaagatgaa gataagagtg agcctgagca gacaggggct gatgctgcag
  1261 aagatgagga agtggatgtc accagcgtgg accctattgc tgccttctgt agcagtgatg
  1321 aagagctgga agattcaaag gctctattgt atttacccat tgcccctgag gtagaagacc
  1381 cagaagaaaa tccttacggc cccccaccgg atgcagtcca aacctccttg atggatgaag
  1441 gggctagttc agagaagaag aggcagtcct cagcagatgg gtcccagcca cctaagaaga
  1501 aacccaaaac aaccaatata gaacttcaag gagtaccaaa tgatgaagtc catccactac
  1561 tgggtgtgaa gggggatggc aaatccaaga agaagcaagc aggccggcct aaaggatcaa
  1621 aaggtaaaga gaaagattct ccatttaaac cgaaactcta caaaggggac agaggtttac
  1681 ctctggaagg atcagcgaag ggtaaagtgc aggcggaact cagccagccc ttgacagcag
  1741 gaggagcaat ctcagaactg ttatgaagac cttcgaagtt cttcattctt tctcactttg
  1801 ccatcatgtg gcctctggac actgtggtca gtcatttgaa aattgacttt aatttaaaac
  1861 aaaggcctgt gcctcccacc caggaggtgg gagggtgaat tttatgttta aatgaagaag
  1921 tgaattatgg aagaagagta tacgaccttc ccttcccttt caagcataag tccaaataga
  1981 ctctcaggaa tgaagatttg tgaagacatc agataggaat tttgactcat ttaaactttg
  2041 atgcttagtt atgttgctgg agaaaagata cttatgtttt gctcatctaa cttcattgta
  2101 cccagcgtca ttttgacatg tcatttccta tctcccattt gccttcggtc ctcaatgcat
  2161 gtctttgagt gacttcttat ctgaaatttt gctactggta tcctaggaaa gcttttgttg
  2221 gatactctca ttttaaactt ctcctctccc cagatacctc ctatatttcc atattgtgtg
  2281 caaaggatgg gcagaaaaga aagtgcttga aagatttcaa attttcagaa agggaacaac
  2341 gaaggccctc tcttcctctc ataccacgtt ttgctcaaga agctgggctg taacaattca
  2401 gggttttccc ttgttttcct ctcattgcat gtttccctcc aatattggtt cattgtcatc
  2461 aatcatggtt tttgaagata gctagtttta tccatctcca gcaaagaatc atcaatagtt
  2521 tatattgctt tacctgtgct ggcttccaga gatggaaaca aacccaggtg tctctcaaca
  2581 agctactttt ttactggggt gggggaatct atgcaaggag taaagtaaaa ccatccagaa
  2641 tcaaagcagc aaccacatag ttcaaatcaa agatcaaggt gaattttttg tatcactgcc
  2701 tgtggaaatc tatcctcatc agtcattgca tttttccctg cctatacctg tgctcctttt
  2761 tcttactgtg ttttcagtca cttcctttct gtgaaaggtt gcttagcttt ttttttgaca
  2821 tttgttgttc tttataaaaa taacagattg gatagatgtg tacatttggt gtttgaaatt
  2881 ctctgaaaat cccattagga aaccaggtgt gaaaagggct cagtagcttc tctgagtggc
  2941 gtttttagct gactggaagt gcttaatctg gatcgtcttt tttttttttt tttttttttc
  3001 aatattttaa aaggagaatt taaatactgt gcttactgtg aaatatatca gttggtgagc
  3061 cgggcgtggt gggtcacgcc tgtaatccca gcactttggg aggccaaggc gggttgatca
  3121 cccgaggtca ggagttcaag accagcctgg ccaacgtggt gaaagcctgt atctattaaa
  3181 agacaaaaat tagctgggcg tggtagtaca tgcctgtaat cccagctaca ctggaggctg
  3241 agtcaggaga atcacttgaa cgtgggaggc agaggttgca gtgagtggag atcgcaccac
  3301 tgccctccag cctaggtgac agaatgagac tctatctcaa aaaaaaaaaa aaaatgatat
  3361 cagttggtgg atgctcctat aggtagccaa acacattgat tacctgttag attttaggat
  3421 agaaatcaaa gtagagcacg tcagcaagag cctctttgtc tcactccatc atttaaaacc
  3481 agtatattca gtagttgaag aaagagctct ccctgagtca gttgcaaaac gtctatattt
  3541 ttagatgcca ctactttttt cttaaatatt cattttgaga ctgtcatgag ttagaccagt
  3601 ggttgagatt agtagatggc tcactagaca tgtttttgtt ttgcagacat tatatccatt
  3661 ccagtcctct gcactgtaca ctgcagcagt gtgcaaacta tgggacttag agggtttctg
  3721 ccatctttcc acgtgtgaag tagcttggtt tcctctgcct gtgcatttgg atgtttgtgc
  3781 tatgtccacc tcctaaactg gctactgaga aaatcatctt cagccctgtc agattgtctc
  3841 tggcagtagc tcctaataat tatttatgtt tttggaattt ttttttcaac ttttaaaaaa
  3901 ccttctatcc atttcaattt gaattatttg atttgtacaa tatatgtata ttctcttctt
  3961 cctttttgtc atccctgccc tgccaccccc aaaattttgt ttttaaaaat attctgggct
  4021 gggcatggtg gctcacacct gtaatcccag cacttgggga ggccgaggct ggtggatcat
  4081 ctgaggtcag gtgtttgaga ccagcctggt caacatggtg aaaccccgtc tctactaaaa
  4141 atacaaaaat tagctgggcg tggtggcggg cacctgtaat cctagctact cgggaggctg
  4201 aggcaggaga attgcttgaa tccaggaggc agaggttgca gtgagctgag attgcgccac
  4261 tgcactccag cctgggtgac agagcaagac tccatctcaa aaaaaaaaaa aaaaaaaaat
  4321 ctgtagtttt gtacaagatg agacttagcc ttgggtactt cttgctgaag ctttaatgct
  4381 ttgtaaataa aatcggatgt ttattaaaga aaaaaaaaaa aaa
WDR5 (accession No. NM_017588):
(SEQ ID NO: 132)
        1 gccgcctggc gcccgcccga gctgccgcct tgtcgagctg agtccgcgct cccgcccagg
      61 cggcggccga cgcgacgccc cgagcgcccg gccccgccgc cgcggcccgg cagactgcct
    121 ctgtcaccgg gtccctccac ccttgtctcc tgtgcggcca gcgtcagagc catggcgacg
    181 gaggagaaga agcccgagac cgaggccgcc agagcacagc caaccccttc gtcatccgcc
    241 actcagagca agcctacacc tgtgaagcca aactatgctc taaagttcac ccttgctggc
    301 cacaccaaag cagtgtcctc cgtgaaattc agcccgaatg gagagtggct ggcaagttca
    361 tctgctgata aacttattaa aatttggggc gcgtatgatg ggaaatttga gaaaaccata
    421 tctggtcaca agctgggaat atccgatgta gcctggtcgt cagattctaa ccttcttgtt
    481 tctgcctcag atgacaaaac cttgaagata tgggacgtga gctcgggcaa gtgtctgaaa
    541 accctgaagg gacacagtaa ttatgtcttt tgctgcaact tcaatcccca gtccaacctt
    601 attgtctcag gatcctttga cgaaagcgtg aggatatggg atgtgaaaac agggaagtgc
    661 ctcaagactt tgccagctca ctcggatcca gtctcggccg ttcattttaa tcgtgatgga
    721 tccttgatag tttcaagtag ctatgatggt ctctgtcgca tctgggacac cgcctcaggc
    781 cagtgcctga agacgctcat cgatgacgac aacccccccg tgtcttttgt gaagttctcc
    841 ccgaacggca aatacatcct ggccgccacg ctggacaaca ctctgaagct ctgggactac
    901 agcaagggga agtgcctgaa gacgtacact ggccacaaga atgagaaata ctgcatattt
    961 gccaatttct ctgttactgg tgggaagtgg attgtgtctg gctcagagga taaccttgtt
  1021 tacatctgga accttcagac gaaagagatt gtacagaaac tacaaggcca cacagatgtc
  1081 gtgatctcaa cagcttgtca cccaacagaa aacatcatcg cctctgctgc gctagaaaat
  1141 gacaaaacaa ttaaactgtg gaagagtgac tgctaagtcc ctttgctcct gcccgcgaga
  1201 gactgtcggg aagttgaccc ggattggcaa gaaacagggt gtcttggagg tggtccccca
  1261 gatctgcgcc tgggggtcag gacagggcct gatttgagcc tcctctctga agatgatttg
  1321 gccgagcgga aggtgtggac caccggaaag ttcttaaaag ttgctggtga catttcttgc
  1381 caattctaac actgtctagg gaagagttcc tagtctattg tgttcaaaca gagtcaacaa
  1441 aagtttttaa ttttttatta cagaagggtg aagttcaatt taacatgcgt tgtgtttttt
  1501 cagtaaacgt tctgtatctt tttgatattc catgacccag tgcacgctgt ggcctgtcac
  1561 cgccaccgtg gccccgccag ctggcctccc ctttggccca cgccggccgc ccccattctc
  1621 tgctgcgtag atgccctggc ccagggccct gactcctcca ttcccgccag tagctgttcc
  1681 tagtgtattt tcgtctttct ggaaaacagc attgagtggt tgttttctgt gtaaagagcc
  1741 gtttgtgtct tgggagtttg tggcccacat gccgatagca cggtcatcgc acatgactct
  1801 cccgtttgtc tcagtgtccc tgcaacaagc agcaccgcag actgtaataa aaggtggggt
  1861 tttgtgaatg gttgtggcaa gtgcgtcctt gtgaagctcg tctccatgtg gctttcttgg
  1921 agaaaggctc ccctggggca agagggtgga aggtttcttt ggacaggagg tgctgaggct
  1981 ggctgcacct gctctctgaa gacgccttcc tctctaggtt cattgttcag tgttgctggg
  2041 ggcggggaac gggggtgggg aggttcttag ttgcgaagga gccaagctcc tgatggactt
  2101 gcgttgggat gtgggggaca cctgtggcat ggtaaggctc cctgagtccc ttactccagg
  2161 tcagatgcca gtgggactca tgcgccctat gagggctgca gggccagtgc tgcccctcgg
  2221 actcctcgag gggttgggtg ctaagcgcga gcctcgccgt ccctgctgga gccctcgcct
  2281 gcctgcccct ctgcctgtgc tcctggcagt gtggcttccc ggtgctcacc tgcacagcag
  2341 ttaacagcag aggccgagcg ggagcctctg gggagcgagg ctgaaacctg aacctgccca
  2401 tggagacagt tgtggtgagg gttgccacac acagtgaggg cggagcaggg tggctgaggg
  2461 cacaggtgcc tgggtctgtc ccacggggca gggctttggg gctgtgatgc tctgggaagc
  2521 cagcttgggt cctgggtcta cagagggccc tggccccgga gcccagccag ctctgcctct
  2581 ctcagggcct ggagtcctgg gggagctcag ccagctctgc ctttctcagg gcctggagtc
  2641 ctggatgaat cctgcaggtt tttggttgca ccggcccagg gaggaagcgg ggggtttgtc
  2701 aggtgggctc tcctggaggt cctcgagtgg caggggtgag gaggggatta tctgaggcat
  2761 ctggagatgt atatcctgtg gtttcccctg cccctctgtt tccgatgagg tgtacggatg
  2821 agtgacctgc actaagaagt gagttgccac agtgaaaatg ggttggtttt tgtcttcgac
  2881 gctcagggtc tgggcgcctc gcatttgcag tctgttgtga cagacacggg gagctccgcg
  2941 tgccagcctg tggctgccct gctgtggggg tcctggggcc ggcgaggccc cttcagtctt
  3001 gttctggggg gacggcccac tccggggagg gggtgtgctg tgctgagcgc tgtatccctg
  3061 aatatagttt attttttcta catttgaatt ctgttgtaga tttatgtaaa aatacattct
  3121 ttttgaaaat aaaaattttc atgtcttcta atttaaaaaa aaa
KMT2A (accession No. NM_001197104):
(SEQ ID NO: 133)
        1 ctgcttcact tcacggggcg aacatggcgc acagctgtcg gtggcgcttc cccgcccgac
      61 ccgggaccac cgggggcggc ggcggcgggg ggcgccgggg cctagggggc gccccgcggc
    121 aacgcgtccc ggccctgctg cttccccccg ggcccccggt cggcggtggc ggccccgggg
    181 cgcccccctc ccccccggct gtggcggccg cggcggcggc ggcgggaagc agcggggctg
    241 gggttccagg gggagcggcc gccgcctcag cagcctcctc gtcgtccgcc tcgtcttcgt
    301 cttcgtcatc gtcctcagcc tcttcagggc cggccctgct ccgggtgggc ccgggcttcg
    361 acgcggcgct gcaggtctcg gccgccatcg gcaccaacct gcgccggttc cgggccgtgt
    421 ttggggagag cggcggggga ggcggcagcg gagaggatga gcaattctta ggttttggct
    481 cagatgaaga agtcagagtg cgaagtccca caaggtctcc ttcagttaaa actagtcctc
    541 gaaaacctcg tgggagacct agaagtggct ctgaccgaaa ttcagctatc ctctcagatc
    601 catctgtgtt ttcccctcta aataaatcag agaccaaatc tggagataag atcaagaaga
    661 aagattctaa aagtatagaa aagaagagag gaagacctcc caccttccct ggagtaaaaa
    721 tcaaaataac acatggaaag gacatttcag agttaccaaa gggaaacaaa gaagatagcc
    781 tgaaaaaaat taaaaggaca ccttctgcta cgtttcagca agccacaaag attaaaaaat
    841 taagagcagg taaactctct cctctcaagt ctaagtttaa gacagggaag cttcaaatag
    901 gaaggaaggg ggtacaaatt gtacgacgga gaggaaggcc tccatcaaca gaaaggataa
    961 agaccccttc gggtctcctc attaattctg aactggaaaa gccccagaaa gtccggaaag
  1021 acaaggaagg aacacctcca cttacaaaag aagataagac agttgtcaga caaagccctc
  1081 gaaggattaa gccagttagg attattcctt cttcaaaaag gacagatgca accattgcta
  1141 agcaactctt acagagggca aaaaaggggg ctcaaaagaa aattgaaaaa gaagcagctc
  1201 agctgcaggg aagaaaggtg aagacacagg tcaaaaatat tcgacagttc atcatgcctg
  1261 ttgtcagtgc tatctcctcg cggatcatta agacccctcg gcggtttata gaggatgagg
  1321 attatgaccc tccaattaaa attgcccgat tagagtctac accgaatagt agattcagtg
  1381 ccccgtcctg tggatcttct gaaaaatcaa gtgcagcttc tcagcactcc tctcaaatgt
  1441 cttcagactc ctctcgatct agtagcccca gtgttgatac ctccacagac tctcaggctt
  1501 ctgaggagat tcaggtactt cctgaggagc ggagcgatac ccctgaagtt catcctccac
  1561 tgcccatttc ccagtcccca gaaaatgaga gtaatgatag gagaagcaga aggtattcag
  1621 tgtcggagag aagttttgga tctagaacga cgaaaaaatt atcaactcta caaagtgccc
  1681 cccagcagca gacctcctcg tctccacctc cacctctgct gactccaccg ccaccactgc
  1741 agccagcctc cagtatctct gaccacacac cttggcttat gcctccaaca atccccttag
  1801 catcaccatt tttgcctgct tccactgctc ctatgcaagg gaagcgaaaa tctattttgc
  1861 gagaaccgac atttaggtgg acttctttaa agcattctag gtcagagcca caatactttt
  1921 cctcagcaaa gtatgccaaa gaaggtctta ttcgcaaacc aatatttgat aatttccgac
  1981 cccctccact aactcccgag gacgttggct ttgcatctgg tttttctgca tctggtaccg
  2041 ctgcttcagc ccgattgttt tcgccactcc attctggaac aaggtttgat atgcacaaaa
  2101 ggagccctct tctgagagct ccaagattta ctccaagtga ggctcactct agaatatttg
  2161 agtctgtaac cttgcctagt aatcgaactt ctgctggaac atcttcttca ggagtatcca
  2221 atagaaaaag gaaaagaaaa gtgtttagtc ctattcgatc tgaaccaaga tctccttctc
  2281 actccatgag gacaagaagt ggaaggctta gtagttctga gctctcacct ctcacccccc
  2341 cgtcttctgt ctcttcctcg ttaagcattt ctgttagtcc tcttgccact agtgccttaa
  2401 acccaacttt tacttttcct tctcattccc tgactcagtc tggggaatct gcagagaaaa
  2461 atcagagacc aaggaagcag actagtgctc cggcagagcc attttcatca agtagtccta
  2521 ctcctctctt cccttggttt accccaggct ctcagactga aagagggaga aataaagaca
  2581 aggcccccga ggagctgtcc aaagatcgag atgctgacaa gagcgtggag aaggacaaga
  2641 gtagagagag agaccgggag agagaaaagg agaataagcg ggagtcaagg aaagagaaaa
  2701 ggaaaaaggg atcagaaatt cagagtagtt ctgctttgta tcctgtgggt agggtttcca
  2761 aagagaaggt tgttggtgaa gatgttgcca cttcatcttc tgccaaaaaa gcaacagggc
  2821 ggaagaagtc ttcatcacat gattctggga ctgatattac ttctgtgact cttggggata
  2881 caacagctgt caaaaccaaa atacttataa agaaagggag aggaaatctg gaaaaaacca
  2941 acttggacct cggcccaact gccccatccc tggagaagga gaaaaccctc tgcctttcca
  3001 ctccttcatc tagcactgtt aaacattcca cttcctccat aggctccatg ttggctcagg
  3061 cagacaagct tccaatgact gacaagaggg ttgccagcct cctaaaaaag gccaaagctc
  3121 agctctgcaa gattgagaag agtaagagtc ttaaacaaac cgaccagccc aaagcacagg
  3181 gtcaagaaag tgactcatca gagacctctg tgcgaggacc ccggattaaa catgtctgca
  3241 gaagagcagc tgttgccctt ggccgaaaac gagctgtgtt tcctgatgac atgcccaccc
  3301 tgagtgcctt accatgggaa gaacgagaaa agattttgtc ttccatgggg aatgatgaca
  3361 agtcatcaat tgctggctca gaagatgctg aacctcttgc tccacccatc aaaccaatta
  3421 aacctgtcac tagaaacaag gcaccccagg aacctccagt aaagaaagga cgtcgatcga
  3481 ggcggtgtgg gcagtgtccc ggctgccagg tgcctgagga ctgtggtgtt tgtactaatt
  3541 gcttagataa gcccaagttt ggtggtcgca atataaagaa gcagtgctgc aagatgagaa
  3601 aatgtcagaa tctacaatgg atgccttcca aagcctacct gcagaagcaa gctaaagctg
  3661 tgaaaaagaa agagaaaaag tctaagacca gtgaaaagaa agacagcaaa gagagcagtg
  3721 ttgtgaagaa cgtggtggac tctagtcaga aacctacccc atcagcaaga gaggatcctg
  3781 ccccaaagaa aagcagtagt gagcctcctc cacgaaagcc cgtcgaggaa aagagtgaag
  3841 aagggaatgt ctcggcccct gggcctgaat ccaaacaggc caccactcca gcttccagga
  3901 agtcaagcaa gcaggtctcc cagccagcac tggtcatccc gcctcagcca cctactacag
  3961 gaccgccaag aaaagaagtt cccaaaacca ctcctagtga gcccaagaaa aagcagcctc
  4021 caccaccaga atcaggtcca gagcagagca aacagaaaaa agtggctccc cgcccaagta
  4081 tccctgtaaa acaaaaacca aaagaaaagg aaaaaccacc tccggtcaat aagcaggaga
  4141 atgcaggcac tttgaacatc ctcagcactc tctccaatgg caatagttct aagcaaaaaa
  4201 ttccagcaga tggagtccac aggatcagag tggactttaa ggaggattgt gaagcagaaa
  4261 atgtgtggga gatgggaggc ttaggaatct tgacttctgt tcctataaca cccagggtgg
  4321 tttgctttct ctgtgccagt agtgggcatg tagagtttgt gtattgccaa gtctgttgtg
  4381 agcccttcca caagttttgt ttagaggaga acgagcgccc tctggaggac cagctggaaa
  4441 attggtgttg tcgtcgttgc aaattctgtc acgtttgtgg aaggcaacat caggctacaa
  4501 agcagctgct ggagtgtaat aagtgccgaa acagctatca ccctgagtgc ctgggaccaa
  4561 actaccccac caaacccaca aagaagaaga aagtctggat ctgtaccaag tgtgttcgct
  4621 gtaagagctg tggatccaca actccaggca aagggtggga tgcacagtgg tctcatgatt
  4681 tctcactgtg tcatgattgc gccaagctct ttgctaaagg aaacttctgc cctctctgtg
  4741 acaaatgtta tgatgatgat gactatgaga gtaagatgat gcaatgtgga aagtgtgatc
  4801 gctgggtcca ttccaaatgt gagaatcttt caggtacaga agatgagatg tatgagattc
  4861 tatctaatct gccagaaagt gtggcctaca cttgtgtgaa ctgtactgag cggcaccctg
  4921 cagagtggcg actggccctt gaaaaagagc tgcagatttc tctgaagcaa gttctgacag
  4981 ctttgttgaa ttctcggact accagccatt tgctacgcta ccggcaggct gccaagcctc
  5041 cagacttaaa tcccgagaca gaggagagta taccttcccg cagctccccc gaaggacctg
  5101 atccaccagt tcttactgag gtcagcaaac aggatgatca gcagccttta gatctagaag
  5161 gagtcaagag gaagatggac caagggaatt acacatctgt gttggagttc agtgatgata
  5221 ttgtgaagat cattcaagca gccattaatt cagatggagg acagccagaa attaaaaaag
  5281 ccaacagcat ggtcaagtcc ttcttcattc ggcaaatgga acgtgttttt ccatggttca
  5341 gtgtcaaaaa gtccaggttt tgggagccaa ataaagtatc aagcaacagt gggatgttac
  5401 caaacgcagt gcttccacct tcacttgacc ataattatgc tcagtggcag gagcgagagg
  5461 aaaacagcca cactgagcag cctcctttaa tgaagaaaat cattccagct cccaaaccca
  5521 aaggtcctgg agaaccagac tcaccaactc ctctgcatcc tcctacacca ccaattttga
  5581 gtactgatag gagtcgagaa gacagtccag agctgaaccc acccccaggc atagaagaca
  5641 atagacagtg tgcgttatgt ttgacttatg gtgatgacag tgctaatgat gctggtcgtt
  5701 tactatatat tggccaaaat gagtggacac atgtaaattg tgctttgtgg tcagcggaag
  5761 tgtttgaaga tgatgacgga tcactaaaga atgtgcatat ggctgtgatc aggggcaagc
  5821 agctgagatg tgaattctgc caaaagccag gagccaccgt gggttgctgt ctcacatcct
  5881 gcaccagcaa ctatcacttc atgtgttccc gagccaagaa ctgtgtcttt ctggatgata
  5941 aaaaagtata ttgccaacga catcgggatt tgatcaaagg cgaagtggtt cctgagaatg
  6001 gatttgaagt tttcagaaga gtgtttgtgg actttgaagg aatcagcttg agaaggaagt
  6061 ttctcaatgg cttggaacca gaaaatatcc acatgatgat tgggtctatg acaatcgact
  6121 gcttaggaat tctaaatgat ctctccgact gtgaagataa gctctttcct attggatatc
  6181 agtgttccag ggtatactgg agcaccacag atgctcgcaa gcgctgtgta tatacatgca
  6241 agatagtgga gtgccgtcct ccagtcgtag agccggatat caacagcact gttgaacatg
  6301 atgaaaacag gaccattgcc catagtccaa catcttttac agaaagttca tcaaaagaga
  6361 gtcaaaacac agctgaaatt ataagtcctc catcaccaga ccgacctcct cattcacaaa
  6421 cctctggctc ctgttattat catgtcatct caaaggtccc caggattcga acacccagtt
  6481 attctccaac acagagatcc cctggctgtc gaccgttgcc ttctgcagga agtcctaccc
  6541 caaccactca tgaaatagtc acagtaggtg atcctttact ctcctctgga cttcgaagca
  6601 ttggctccag gcgtcacagt acctcttcct tatcacccca gcggtccaaa ctccggataa
  6661 tgtctccaat gagaactggg aatacttact ctaggaataa tgtttcctca gtctccacca
  6721 ccgggaccgc tactgatctt gaatcaagtg ccaaagtagt tgatcatgtc ttagggccac
  6781 tgaattcaag tactagttta gggcaaaaca cttccacctc ttcaaatttg caaaggacag
  6841 tggttactgt aggcaataaa aacagtcact tggatggatc ttcatcttca gaaatgaagc
  6901 agtccagtgc ttcagacttg gtgtccaaga gctcctcttt aaagggagag aagaccaaag
  6961 tgctgagttc caagagctca gagggatctg cacataatgt ggcttaccct ggaattccta
  7021 aactggcccc acaggttcat aacacaacat ctagagaact gaatgttagt aaaatcggct
  7081 cctttgctga accctcttca gtgtcgtttt cttctaaaga ggccctctcc ttcccacacc
  7141 tccatttgag agggcaaagg aatgatcgag accaacacac agattctacc caatcagcaa
  7201 actcctctcc agatgaagat actgaagtca aaaccttgaa gctatctgga atgagcaaca
  7261 gatcatccat tatcaacgaa catatgggat ctagttccag agataggaga cagaaaggga
  7321 aaaaatcctg taaagaaact ttcaaagaaa agcattccag taaatctttt ttggaacctg
  7381 gtcaggtgac aactggtgag gaaggaaact tgaagccaga gtttatggat gaggttttga
  7441 ctcctgagta tatgggccaa cgaccatgta acaatgtttc ttctgataag attggtgata
  7501 aaggcctttc tatgccagga gtccccaaag ctccacccat gaagtagaa ggatctgcca
  7561 aggaattaca ggcaccacgg aaacgcacag tcaaagtgac actgacacct ctaaaaatgg
  7621 aaaatgagag tcaatccaaa aatgccctga aagaaagtag tcctgcttcc cctttgcaaa
  7681 tagagtcaac atctcccaca gaaccaattt cagcctctga aaatccagga gatggtccag
  7741 tggcccaacc aagccccaat aatacctcat gccaggattc tcaaagtaac aactatcaga
  7801 atcttccagt acaggacaga aacctaatgc ttccagatgg ccccaaacct caggaggatg
  7861 gctcttttaa aaggaggtat ccccgtcgca gtgcccgtgc acgttctaac atgttttttg
  7921 ggcttacccc actctatgga gtaagatcct atggtgaaga agacattcca ttctacagca
  7981 gctcaactgg gaagaagcga ggcaagagat cagctgaagg acaggtggat ggggccgatg
  8041 acttaagcac ttcagatgaa gacgacttat actattacaa cttcactaga acagtgattt
  8101 cttcaggtgg agaggaacga ctggcatccc ataatttatt tcgggaggag gaacagtgtg
  8161 atcttccaaa aatctcacag ttggatggtg ttgatgatgg gacagagagt gatactagtg
  8221 tcacagccac aacaaggaaa agcagccaga ttccaaaaag aaatggtaaa gaaaatggaa
  8281 cagagaactt aaagattgat agacctgaag atgctgggga gaaagaacat gtcactaaga
  8341 gttctgttgg ccacaaaaat gagccaaaga tggataactg ccattctgta agcagagtta
  8401 aaacacaggg acaagattcc ttggaagctc agctcagctc attggagtca agccgcagag
  8461 tccacacaag taccccctcc gacaaaaatt tactggacac ctataatact gagctcctga
  8521 aatcagattc agacaataac aacagtgatg actgtgggaa tatcctgcct tcagacatta
  8581 tggactttgt actaaagaat actccatcca tgcaggcttt gggtgagagc ccagagtcat
  8641 cttcatcaga actcctgaat cttggtgaag gattgggtct tgacagtaat cgtgaaaaag
  8701 acatgggtct ttttgaagta ttttctcagc agctgcctac aacagaacct gtggatagta
  8761 gtgtctcttc ctctatctca gcagaggaac agtttgagtt gcctctagag ctaccatctg
  8821 atctgtctgt cttgaccacc cggagtccca ctgtccccag ccagaatccc agtagactag
  8881 ctgttatctc agactcaggg gagaagagag taaccatcac agaaaaatct gtagcctcct
  8941 ctgaaagtga cccagcactg ctgagcccag gagtagatcc aactcctgaa ggccacatga
  9001 ctcctgatca ttttatccaa ggacacatgg atgcagacca catctctagc cctccttgtg
  9061 gttcagtaga gcaaggtcat ggcaacaatc aggatttaac taggaacagt agcacccctg
  9121 gccttcaggt acctgtttcc ccaactgttc ccatccagaa ccagaagtat gtgcccaatt
  9181 ctactgatag tcctggcccg tctcagattt ccaatgcagc tgtccagacc actccacccc
  9241 acctgaagcc agccactgag aaactcatag ttgttaacca gaacatgcag ccactttatg
  9301 ttctccaaac tcttccaaat ggagtgaccc aaaaaatcca attgacctct tctgttagtt
  9361 ctacacccag tgtgatggag acaaatactt cagtattggg acccatggga ggtggtctca
  9421 cccttaccac aggactaaat ccaagcttgc caacttctca atctttgttc ccttctgcta
  9481 gcaaaggatt gctacccatg tctcatcacc agcacttaca ttccttccct gcagctactc
  9541 aaagtagttt cccaccaaac atcagcaatc ctccttcagg cctgcttatt ggggttcagc
  9601 ctcctccgga tccccaactt ttggtttcag aatccagcca gaggacagac ctcagtacca
  9661 cagtagccac tccatcctct ggactcaaga aaagacccat atctcgtcta cagacccgaa
  9721 agaataaaaa acttgctccc tctagtaccc cttcaaacat tgccccttct gatgtggttt
  9781 ctaatatgac attgattaac ttcacaccct cccagcttcc taatcatcca agtctgttag
  9841 atttggggtc acttaatact tcatctcacc gaactgtccc caacatcata aaaagatcta
  9901 aatctagcat catgtatttt gaaccggcac ccctgttacc acagagtgtg ggaggaactg
  9961 ctgccacagc ggcaggcaca tcaacaataa gccaggatac tagccacctc acatcagggt
10021 ctgtgtctgg cttggcatcc agttcctctg tcttgaatgt tgtatccatg caaactacca
10081 caacccctac aagtagtgcg tcagttccag gacacgtcac cttaaccaac ccaaggttgc
10141 ttggtacccc agatattggc tcaataagca atcttttaat caaagctagc cagcagagcc
10201 tggggattca ggaccagcct gtggctttac cgccaagttc aggaatgttt ccacaactgg
10261 ggacatcaca gaccccctct actgctgcaa taacagcggc atctagcatc tgtgtgctcc
10321 cctccactca gactacgggc ataacagccg cttcaccttc tggggaagca gacgaacact
10381 atcagcttca gcatgtgaac cagctccttg ccagcaaaac tgggattcat tcttcccagc
10441 gtgatcttga ttctgcttca gggccccagg tatccaactt tacccagacg gtagacgctc
10501 ctaatagcat gggactggag cagaacaagg ctttatcctc agctgtgcaa gccagcccca
10561 cctctcctgg gggttctcca tcctctccat cttctggaca gcggtcagca agcccttcag
10621 tgccgggtcc cactaaaccc aaaccaaaaa ccaaacggtt tcagctgcct ctagacaaag
10681 ggaatggcaa gaagcacaaa gtttcccatt tgcggaccag ttcttctgaa gcacacattc
10741 cagaccaaga aacgacatcc ctgacctcag gcacagggac tccaggagca gaggctgagc
10801 agcaggatac agctagcgtg gagcagtcct cccagaagga gtgtgggcaa cctgcagggc
10861 aagtcgctgt tcttccggaa gttcaggtga cccaaaatcc agcaaatgaa caagaaagtg
10921 cagaacctaa aacagtggaa gaagaggaaa gtaatttcag ctccccactg atgctttggc
10981 ttcagcaaga acaaaagcgg aaggaaagca ttactgagaa aaaacccaag aaaggacttg
11041 tttttgaaat ttccagtgat gatggctttc agatctgtgc agaaagtatt gaagatgcct
11101 ggaagtcatt gacagataaa gtccaggaag ctcgatcaaa tgcccgccta aagcagctct
11161 catttgcagg tgttaacggt ttgaggatgc tggggattct ccatgatgca gttgtgttcc
11221 tcattgagca gctgtctggt gccaagcact gtcgaaatta caaattccgt ttccacaagc
11281 cagaggaggc caatgaaccc cccttgaacc ctcacggctc agccagggct gaagtccacc
11341 tcaggaagtc agcatttgac atgtttaact tcctggcttc taaacatcgt cagcctcctg
11401 aatacaaccc caatgatgaa gaagaggagg aggtacagct gaagtcagct cggagggcaa
11461 ctagcatgga tctgccaatg cccatgcgct tccggcactt aaaaaagact tctaaggagg
11521 cagttggtgt ctacaggtct cccatccatg gccggggtct tttctgtaag agaaacattg
11581 atgcaggtga gatggtgatt gagtatgccg gcaacgtcat ccgctccatc cagactgaca
11641 agcgggaaaa gtattacgac agcaagggca ttggttgcta tatgttccga attgatgact
11701 cagaggtagt ggatgccacc atgcatggaa atgctgcacg cttcatcaat cactcgtgtg
11761 agcctaactg ctattctcgg gtcatcaata ttgatgggca gaagcacatt gtcatctttg
11821 ccatgcgtaa gatctaccga ggagaggaac tcacttacga ctataagttc cccattgagg
11881 atgccagcaa caagctgccc tgcaactgtg gcgccaagaa atgccggaag ttcctaaact
11941 aaagctgctc ttctccccca gtgttggagt gcaaggaggc ggggccatcc aaagcaacgc
12001 tgaaggcctt ttccagcagc tcggagctcc cggattgcgt ggcacagctg aggggcctct
12061 gtgatggctg agctctctta tgtcctatac tcacatcaga catgtgatca tagtcccaga
12121 gacagagttg aggtctcgaa gaaaagatcc atgatcggct ttctcctggg gcccctccaa
12181 ttgtttactg ttagaaagtg ggaatggggt ccctagcaga cttgcctgga aggagcctat
12241 tatagagggt tggttatgtt gggagattgg gcctgaattt ctccacagaa ataagttgcc
12301 atcctcaggt tggccctttc ccaagcactg taagtgagtg ggtcaggcaa agccccaaat
12361 ggagggttgg ttagattcct gacagtttgc cagccaggcc ccacctacag cgtctgtcga
12421 acaaacagag gtctggtggt tttccctact atcctcccac tcgagagttc acttctggtt
12481 gggagacagg attcctagca cctccggtgt caaaaggctg tcatggggtt gtgccaatta
12541 attaccaaac attgagcctg caggctttga gtgggagtgt tgcccccagg agccttatct
12601 cagccaatta cctttcttga cagtaggagc ggcttccctc tcccattccc tcttcactcc
12661 cttttcttcc tttcccctgt cttcatgcca ctgctttccc atgcttcttt cgggttgtag
12721 gggagactga ctgcctgctc aaggacactc cctgctgggc ataggatgtg cctgcaaaaa
12781 gttccctgag cctgtaagca ctccaggtgg ggaagtggac aggagccatt ggtcataacc
12841 agacagaatt tggaaacatt ttcataaagc tccatggaga gttttaaaga aacatatgta
12901 gcatgatttt gtaggagagg aaaaagatta tttaaatagg atttaaatca tgcaacaacg
12961 agagtatcac agccaggatg acccttgggt cccattccta agacatggtt actttatttt
13021 ccccttgtta agacatagga agacttaatt tttaaacggt cagtgtccag ttgaaggcag
13081 aacactaatc agatttcaag gcccacaact tggggactag accaccttat gttgagggaa
13141 ctctgccacc tgcgtgcaac ccacagctaa agtaaattca atgacactac tgccctgatt
13201 actccttagg atgtggtcaa aacagcatca aatgtttctt ctcttccttt ccccaagaca
13261 gagtcctgaa cctgttaaat taagtcattg gattttactc tgttctgttt acagtttact
13321 atttaaggtt ttataaatgt aaatatattt tgtatatttt tctatgagaa gcacttcata
13381 gggagaagca cttatgacaa ggctattttt taaaccgcgg tattatccta atttaaaaga
13441 agatcggttt ttaataattt tttattttca taggatgaag ttagagaaaa tattcagctg
13501 tacacacaaa gtctggtttt tcctgcccaa cttccccctg gaaggtgtac tttttgttgt
13561 ttaatgtgta gcttgtttgt gccctgttga cataaatgtt tcctgggttt gctctttgac
13621 aataaatgga gaaggaaggt cacccaactc cattgggcca ctcccctcct tcccctattg
13681 aagctcctca aaaggctaca gtaatatctt gatacaacag attctcttct ttcccgcctc
13741 tctcctttcc ggcgcaactt ccagagtggt gggagacggc aatctttaca tttccctcat
13801 ctttcttact tcagagttag caaacaacaa gttgaatggc aacttgacat ttttgcatca
13861 ccatctgcct cataggccac tctttccttt ccctctgccc accaagtcct catatctgca
13921 gagaacccat tgatcacctt gtgccctctt ttggggcagc ctgttgaaac tgaagcacag
13981 tctgaccact cacgataaag cagatttttc tctgcctctg ccacaaggtt tcagagtagt
14041 gtagtccaag tagagggtgg ggcacccttt tctcgccgca agaagcccat tcctatggaa
14101 gtctagcaaa gcaatacgac tcagcccagc actctctgcc ccaggactca tggctctgct
14161 gtgccttcca tcctgggctc ccttctctcc tgtgacctta agaactttgt ctggtggctt
14221 tgctggaaca ttgtcactgt tttcactgtc atgcagggag cccagcactg tggccaggat
14281 ggcagagact tccttgtcat catggagaag tgccagcagg ggactgggaa aagcactcta
14341 cccagacctc acctcccttc ctccttttgc ccatgaacaa gatgcagtgg ccctaggggt
14401 tccactagtg tctgctttcc tttattattg cactgtgtga ggtttttttg taaatccttg
14461 tattcctatt ttttttaaag aaaaaaaaaa aaccttaagc tgcatttgtt actgaaatga
14521 ttaatgcact gatgggtcct gaattcacct tgagaaagac ccaaaggcca gtcagggggt
14581 ggggggaact cagctaaata gacctagtta ctgccctgct aggccatgct gtactgtgag
14641 cccctcctca ctctctacca accctaaacc ctgaggacag gggaggaacc cacagcttcc
14701 ttctcctgcc agctgcagat ggtttgcctt gcctttccac cccctaattg tcaaccacaa
14761 aaatgagaaa ttcctcttct agctcagcct tgagtccatt gccaaatttt cagcacacct
14821 gccagcaact tgggggaata agcgaaggtt tccctacaag agggaaagaa ggcaaaaacg
14881 gcacagctat ctccaaacac atctgagttc atttcaaaag tgaccaaggg aatctccgca
14941 caaaagtgca gattgaggaa ttgtgatggg tcattcccaa gaatccccca aggggcatcc
15001 caaatccctg aggagtaaca gctgcaaacc tggtcagttc tcagtgagag ccagctcact
15061 tatagctttg ctgctagaac ctgttgtggc tgcatttcct ggtggccagt gacaactgtg
15121 taaccagaat agctgcatgg cgctgaccct ttggccggaa cttggtctct tggctccctc
15181 cttggccacc caccacctct cgcacagccc ctctgttttt acaccaataa caagaattaa
15241 gggggaagcc ctggcagcta tacgttttca accagactcc tttgccggga cccagcccgc
15301 caccctgctc gcctccgtca aacccccggc caatgcagtg agcaccatgt agctcccttg
15361 atttaaaaaa aataaaaaat aaaaaaaaaa ggaaaaaaaa atacaacaca cacacaaaaa
15421 taaaaaaaat attctaatga atgtatcttt ctaaaggact gacgttcaat caaatatctg
15481 aaaatactaa aggtcaaaac cttgtcagat gttaacttct aagttcggtt tgggattttt
15541 tttttttaat agaaatcaag ttgtttttgt ttttaaggaa aagcgggtca ttgcaaaggg
15601 ctgggtgtaa ttttatgttt catttccttc attttaaagc aatacaaggt tatggagcag
15661 atggttttgt gccgaatcat gaatactagt caagtcacac actctggaaa cttgcaactt
15721 tttgtttgtt ttggttttca aataaatata aatatgatat atataggaac taatatagta
15781 atgcaccatg taacaaagcc tagttcagtc catggctttt aattctctta acactataga
15841 taaggattgt gttacagttg ctagtagcgg caggaagatg tcaggctcac tttcctctga
15901 ttcccgaaat ggggggaacc tctaaccata aaggaatggt agaacagtcc attcctcgga
15961 tcagagaaaa atgcagacat ggtgtcacct ggattttttt ctgcccatga atgttgccag
16021 tcagtacctg tcctccttgt ttctctattt ttggttatga atgttggggt taccacctgc
16081 atttagggga aaattgtgtt ctgtgctttc ctggtatctt gttccgaggt actctagttc
16141 tgtctttcaa ccaagaaaat agaattgtgg tgtttctttt attgaacttt taacagtctc
16201 tttagtaaat acaggtagtt gaataattgt ttcaagagct caacagatga caagcttctt
16261 ttctagaaat aagacatttt ttgacaactt tatcatgtat aacagatctg ttttttttcc
16321 ttgtgttctt ccaagcttct ggttagagaa aaagagaaaa aaaaaaaagg aaaatgtgtc
16381 taaagtccat cagtgttaac tccctgtgac agggatgaag gaaaatactt taatagttca
16441 aaaaataata atgctgaaag ctctctacga aagactgaat gtaaaagtaa aaagtgtaca
16501 tagttgtaaa aaaaaggagt ttttaaacat gtttattttc tatgcacttt tttttattta
16561 agtgatagtt taattaataa acatgtcaag tttaaaaaaa aaaaaaaa
KMT2D (accession No. XM_006719616):
(SEQ ID NO: 134)
        1 agcggaagga tcccgcagcg tgtgcgtaga actgcagagt cacagccttt cctccgagag
      61 ggcgggatcc ctccgccgct ccgctccaac acaaaatagg gccgcctctt ctcctttctc
    121 cccctctcga gtggggtgcc ccgggcaaaa ggcccccccg gatctagcgc cgaaggcttc
    181 acgaatcttc acgaccgctg cccagctctt gggccaggaa atagcccctt cgcaggaacc
    241 accctaccgg ccgaacagga ggcggagggg gggaggcgga gcggcgccgc gctgcactac
    301 tttcctctcc ggttgcaaat ggctgcctcg ttccccactt tccgctcagt ttcctgaccc
    361 cccggtgccg ggagccgggg ttgggccatg cacctctagg ccgcctgcga tcacagtcag
    421 ccgggggtcg agggggtgcc accgaccaga gccggccagg ccgggggcgg ggcagctccc
    481 gaggccagag gggaagggag gcgagcgcag ggcctggagc ggccggaggg gagcgggcag
    541 agggctcgca ccgcccgccc cttccttttc ctcgcctacc tagcctcctc ccttccccgg
    601 ggggagcaga aggtgggggg ctcgaagccg ccgagggtga gcgctcgggg tcgagaagcc
    661 cggcgctggg tgtgtgtcag gttcagcccc gcggccccgc cggccccgcg tcgccgtagc
    721 tcgcgcggcc ccgggcgccg gccggggcgg ggagaggggc tcggcgctcc tgcgagggtc
    781 tcacgttcca tccgggccag gcgcggggcg gcgcggcatt ccttccgggc tgctggggag
    841 gcgcctcgac gttccatctg gagagcctcg acgttccgcc cgagcccggc gcgggcggcc
    901 ggggcgctgg ccgggcccta ggactgagag gccgcccggc gacgcggatg cggagcctgc
    961 tcgcccaaga tcaaagccac cggtgctctc tttgtgtccg ctcgggattc gccgccctgg
  1021 ggctgtccat ggaaacctaa actgctggaa cctgaggcag agaacccctc tttggcttct
  1081 tgctgttttt ttgtgggggg agggtggcca ctccgacctg gatttaccgt tcttggcccc
  1141 cctaagcccc cccgtgcggg gggcggctgt gatcgctctg gcggttggag gtcggggagc
  1201 ggcccgggct ctggccatgt tctcggatga ggatttctgg atcgccctgt gaagaggtct
  1261 ccccgagagg gccctgccca gtcggagaga gggatggaca gccagaagct ggctggtgag
  1321 gataaagatt cagaaccggc agctgatgga cctgcagctt ctgaggaccc aagtgccact
  1381 gagtcagacc tgcccaaccc acatgtggga gaggtctctg tccttagttc tgggagtccc
  1441 aggcttcagg agactcctca ggactgcagt gggggtccgg tgcggcgttg tgctctctgt
  1501 aactgcgggg agcccagtct acacgggcag cgggagctac ggcgctttga gttgccattt
  1561 gattggcccc ggtgtccagt ggtgtcccct ggggggagcc cagggcccaa tgaggcagtg
  1621 ctgcccagtg aggacctatc acagattggt ttccctgagg gccttacacc tgcccaccta
  1681 ggagaacctg gagggtcctg ctgggctcac cattggtgtg ctgcatggtc ggcaggcgta
  1741 tgggggcagg agggcccaga actatgtggt gtggacaagg ccatcttctc agggatctca
  1801 cagcgctgct cccactgcac caggctcggt gcctccatcc cttgccgctc acctggatgt
  1861 ccacggcttt accacttccc ctgcgcgact gccagcggtt ccttcctatc catgaaaaca
  1921 ctgcagctgc tatgcccaga gcacagtgag ggggctgcat atctggagga ggctcgctgt
  1981 gcagtgtgtg aggggccagg ggagttgtgt gacctgttct tctgtaccag ctgtgggcat
  2041 cactatcacg gggcctgcct ggacactgct ctgactgccc gcaaacgtgc tggctggcag
  2101 tgccctgaat gcaaagtgtg ccaagcctgc aggaaacctg ggaatgactc taagatgttg
  2161 gtttgtgaga cgtgtgacaa aggataccat actttctgcc taaaaccacc catggaggaa
  2221 ctgcctgctc actcttggaa gtgcaaggcg tgccgggtgt gccgggcctg tggggcgggc
  2281 tcagcagaac tgaatcccaa ctcggagtgg tttgagaact actctctctg tcaccgctgt
  2341 cacaaagccc agggaggtca gactatccgc tccgttgctg agcagcatac cccggtgtgt
  2401 agcagatttt cacccccaga gcctggcgat acccccactg acgagcccga tgctctgtac
  2461 gttgcatgcc aagggcagcc aaagggtggg cacgtgacct ctatgcaacc caaggaacca
  2521 gggcccctgc aatgtgaagc caaaccacta gggaaagcag gggtccaact tgagccccag
  2581 ttggaggccc ccctaaacga ggagatgcca ctgctgcccc cacctgagga gtcacccctg
  2641 tccccaccac ctgaggaatc acccacgtcc ccaccacctg aggcatcacg cctgtcacca
  2701 ccacctgagg aattgcccgc atccccactt cctgaggcat tgcacctgtc ccggccgctg
  2761 gaggaatcgc ccctctctcc gccgcctgag gagtctcctc tgtctccccc acctgaatca
  2821 tcaccttttt ctccactgga ggagtcgccc ttgtctccac cggaagagtc acccccatct
  2881 cctgcacttg agacgcctct atccccacca cctgaagcat cgcccctgtc cccaccattt
  2941 gaagaatctc ctttgtcccc gccacctgag gaattgccca cttccccgcc acctgaagca
  3001 tctcgcctgt ctccaccacc tgaggagtca cccatgtccc ctccacctga agagtcaccc
  3061 atgtctccac caccggaggc atctcgtctg ttcccaccat ttgaagagtc tcctctgtcc
  3121 cctccacctg aggagtctcc cctttcccca ccacctgagg catcacgcct gtccccacca
  3181 cctgaggact cgcctatgtc cccaccacct gaagaatcac ctatgtcccc cccacctgag
  3241 gtatcgcgcc tatcccccct gcctgtggtg tcacgcctgt ctccaccgcc tgaggaatct
  3301 cccttgtccc caccgcctga ggagtctccc acgtcccctc cacctgaggc ttcacgcctc
  3361 tccccaccac ctgaggactc ccccacatcc ccaccacctg aggactcacc tgcttcccca
  3421 ccaccggagg actcgctcat gtccctgccg ctggaggagt cacccctgtt gccactacct
  3481 gaggagccgc aactctgccc ccggtccgag gggccgcacc tgtcaccccg gcctgaggag
  3541 ccgcacctgt ccccccggcc tgaggagcca cacctatctc cgcaggctga ggagccacac
  3601 ctgtcccccc agcctgagga gccatgccta tgcgctgtgc ctgaggagcc acacttgtcc
  3661 ccccaggctg agggaccaca tctgtcccct cagcctgagg aattgcacct gtccccccag
  3721 actgaggagc cgcacctgtc tcctgtgcct gaggagccat gcttgtcccc ccaacctgag
  3781 gaatcacacc tgtcccccca gtctgaggag ccatgcctgt ccccccggcc tgaggaatcg
  3841 catctgtccc ctgagcttga gaagccaccc ctgtcccctc ggcctgaaaa gccccctgag
  3901 gagccaggcc aatgccctgc acctgaggag ctgcccttgt tccctccccc tggggaacca
  3961 tccttatctc ccttgcttgg agagccagcc ctgtctgagc ctggggaacc acctctgtcc
  4021 cctctgcccg aggagctgcc gttgtcccca tctggggagc catccttgtc gcctcagctg
  4081 atgccaccag atccccttcc tcctccactc tcacccatca tcacagctgc ggccccaccg
  4141 gccctgtctc ctttggggga gttagagtac ccctttggtg ccaaagggga cagtgaccct
  4201 gagtcaccgt tggctgcccc catcctggag acacccatca gccctccacc agaagctaac
  4261 tgcactgacc ctgagcctgt cccccctatg atccttcccc catctccagg ctccccagtg
  4321 gggccggctt ctcccatcct gatggagccc cttcctcctc agtgttcgcc actccttcag
  4381 cattccctgg ttccccaaaa ctcccctcct tcccagtgct ctcctcctgc cctaccactg
  4441 tccgttccct ccccgttgag tcccataggg aaggtagtgg gggtctcaga tgaggctgag
  4501 ctgcacgaga tggagactga gaaagtttca gaacctgaat gcccagcctt ggaacccagt
  4561 gccaccagtc ctctcccttc cccaatgggg gacctttcct gccccgcccc cagccctgcc
  4621 ccagccctgg atgacttctc tggcctaggg gaagacacag cccctctgga tgggattgat
  4681 gctccgggtt cacagccaga gcctggacag acccctggca gtttggctag tgaacttaaa
  4741 ggctcccctg tgctcctgga ccccgaggag ctggcccctg tgacccctat ggaggtctac
  4801 cccgaatgca agcagacagc agggcagggc tcaccatgtg aagaacagga agagccacgt
  4861 gcaccggtgg cccccacacc acccactctc atcaaatccg acatcgttaa cgagatctct
  4921 aatctgagcc agggtgatgc cagtgccagt tttcctggct cagagcccct cctgggctct
  4981 ccagacccgg aggggggtgg ctccctgtcc atggagttgg gggtctctac ggatgttagt
  5041 ccagcccgag atgagggctc cctacggctc tgtactgact cactgccaga gactgatgac
  5101 tcactattgt gcgatgctgg gacagctatc agcggaggca aagctgaggg ggagaagggg
  5161 cggcggcgca gctccccagc ccgttcccgc atcaaacagg gtcgcagcag cagtttccca
  5221 ggaagacgcc ggcctcgtgg aggagcccat ggaggacgtg gtagaggacg ggcccggcta
  5281 aagtcaactg cttcttccat tgagactctg gttgctgaca ttgatagctc tcccagtaag
  5341 gaggaggagg aagaagatga tgacaccatg cagaataccg tggttctctt ctccaacaca
  5401 gacaaatttg tcctaatgca ggacatgtgt gtggtatgtg gcagctttgg ccggggggca
  5461 gagggccacc tccttgcctg ttcgcagtgc tctcagtgct atcaccctta ctgtgtcaac
  5521 agcaagatca ccaaggtgat gctgctcaag ggctggcgtt gtgtggagtg tattgtgtgt
  5581 gaggtgtgtg gccaggcctc cgacccctca cgcctgctgc tctgtgatga ctgtgatatt
  5641 agctaccaca catactgcct ggacccccca ctgctcaccg tccccaaggg cggctggaag
  5701 tgcaagtggt gtgtgtcctg tatgcagtgt ggggctgctt cccctggctt ccactgtgaa
  5761 tggcagaata gttacacaca ctgtgggccc tgtgccagcc tggtgacctg ccctatctgt
  5821 catgctcctt acgtagaaga ggacctacta atccagtgcc gccactgtga acggtggatg
  5881 catgcaggct gtgagagcct cttcacagag gacgatgtgg agcaggcagc cgatgaaggc
  5941 tttgactgtg tctcctgcca gccctacgtg gtaaagcctg tggcgcctgt tgcacctcca
  6001 gagctggtgc ccatgaaggt gaaagagcca gagccccagt actttcgctt cgaaggtgtg
  6061 tggctgacag aaactggcat ggccttgctg cgtaacctga ccatgtcacc actgcacaag
  6121 cggcgccaac ggcgaggacg gcttggcctc ccaggcgagg caggattgga gggttctgag
  6181 ccctcagatg cccttggccc tgatgacaag aaggatgggg acctggacac cgatgagctg
  6241 ctcaagggtg aaggtggtgt ggagcacatg gagtgcgaaa ttaaactgga gggccccgtc
  6301 agccctgatg tggagcctgg caaagaggag accgaggaaa gcaaaaaacg caagcgtaaa
  6361 ccatatcggc ctggcattgg tggtttcatg gtgcgacagc ggaaatccca cacacgcacg
  6421 aaaaaggggc ctgctgcaca ggcggaggtg ttgagtgggg atgggcagcc cgacgaggtg
  6481 atacctgctg acctgcctgc agagggcgcc gtggagcaga gcttagctga aggggatgag
  6541 aagaagaagc aacagcggcg agggcgcaag aagagcaaac tggaggacat gttccctgct
  6601 tacttgcagg aagccttctt tgggaaggag ctgctggacc tgagccgtaa ggcccttttt
  6661 gcagttgggg tgggccggcc aagctttgga ctagggaccc caaaagccaa gggagatgga
  6721 ggctcagaaa ggaaggaact ccccacatcg cagaaaggag atgatggtcc agatattgca
  6781 gatgaagaat cccgtggcct cgagggcaaa gccgatacac caggacctga ggatgggggc
  6841 gtgaaggcat ccccagtgcc cagtgaccct gagaagccag gcaccccagg tgaagggatg
  6901 cttagctctg acttagacag gatttccaca gaagaactgc ccaagatgga atccaaggac
  6961 ctgcagcagc tcttcaagga tgttctgggc tctgaacgag aacagcatct gggttgtgga
  7021 acccctggcc tagaaggcag ccgtacgcca ctgcagaggc cctttcttca aggtggactc
  7081 cctttgggca atctgccctc cagcagccca atggactcct acccaggcct ctgccagtcc
  7141 ccgttcctgg attctaggga gcgcgggggc ttctttagcc cggaacccgg tgagcccgac
  7201 agcccctgga cgggctcagg tggcaccacg ccctccaccc ccacaacccc caccacggag
  7261 ggtgagggcg acggactctc ctataaccag cggagtcttc agcgctggga gaaggatgag
  7321 gagttgggcc agctgtccac catctcacct gtgctctatg ccaacattaa ttttcctaat
  7381 ctcaagcaag actacccaga ctggtcaagc cgttgcaaac aaatcatgaa gctctggaga
  7441 aaggttccag cagctgacaa agccccctac ctgcaaaagg ccaaagataa ccgggcagct
  7501 caccgcatca acaaggtgca gaagcaggct gagagccaga tcaacaagca gaccaaggtg
  7561 ggcgacatag cccgtaagac tgaccgaccg gccctacatc tccgcattcc cccgcagcca
  7621 ggggcactgg gcagcccgcc ccccgctgct gcccccacca ttttcattgg cagccccact
  7681 acccccgccg gcttgtctac ctctgcggac gggttcctga agccgccggc gggctcggtg
  7741 cctggccctg actcgcctgg tgagctcttc ctcaagctcc caccccaggt gcccgcccaa
  7801 gtgccttcgc aggacccctt tggactggcc cctgcctatc ccctggagcc ccgcttcccc
  7861 acggcaccgc ccacctatcc cccctatcct agtcctacgg gggcccctgc gcagcccccg
  7921 atgctgggcg cctcatctcg tcctggggct ggccagccag gggaattcca cactacccca
  7981 cctggcaccc ccagacacca gccctccaca cctgacccat tcctcaaacc ccgctgcccc
  8041 tcgctggata acttggctgt gcctgagagc cctggggtag ggggaggcaa agcttccgag
  8101 cccctgctct cgcccccacc ttttggggag tcccggaagg ccctagaggt gaagaaggaa
  8161 gagcttgggg catcctctcc tagctatggg cccccaaacc tgggctttgt tgactcaccc
  8221 tcctcaggca cccacctggg tggcctggag ttaaagacac ctgatgtctt caaagccccc
  8281 ctgacccctc gggcatctca ggtagagccc cagagcccgg gcttgggcct aaggccccag
  8341 gagccacccc ctgcccaggc tttggcacct tctcctccaa gtcacccaga catctttcgc
  8401 cctggctcct acactgaccc atatgctcag cccccattga ctcctcggcc ccaacctccg
  8461 ccccctgaga gctgctgtgc tctgccccct cgctcactgc cctccgaccc tttctcccga
  8521 gtgcctgcca gtcctcagtc ccagtccagc tcccagtctc cactgacacc ccggcctctg
  8581 tctgctgaag ctttttgccc atcacccgtt acccctcgct tccagtcccc tgacccttat
  8641 tctcgcccac cctcacgccc tcagtcccgt gacccatttg ccccattgca taagccaccc
  8701 cgaccccagc cccctgaagt tgcctttaag gctgggtctc tagcccacac ttcgctgggg
  8761 gctggggggt tcccagcagc cctgcccgcg gggccagcag gtgagctcca tgccaaggtc
  8821 ccaagtgggc agccccccaa ttttgtccgg tcccctggga cgggtgcatt tgtgggcacc
  8881 ccctctccca tgcgtttcac tttccctcag gcagtagggg agccttccct aaagccccct
  8941 gtccctcagc ctggtctccc gccaccccat gggatcaaca gccattttgg gcccggcccc
  9001 accttgggca agcctcaaag cacaaactac acagtagcca cagggaactt ccacccatcg
  9061 ggcagccccc tggggcccag cagcgggtcc acaggggaga gctatgggct gtccccacta
  9121 cgccctccgt cggttctgcc accacctgca cccgacggat ccctccccta cctgtcccat
  9181 ggagcctcac agcgatcagg catcacctct cctgtcgaaa agcgagaaga cccagggact
  9241 ggaatgggta gctctttggc gacagctgaa ctcccaggta cccaggaccc aggcatgtcc
  9301 ggccttagcc aaacagagct ggagaagcaa cggcagcgcc agcgactacg agagctgctg
  9361 attcggcagc agatccagcg caacaccctg cggcaggaga aggaaacagc tgcagcagct
  9421 gcaggagcag tggggcctcc aggcagctgg ggtgctgagc ccagcagccc tgcctttgag
  9481 cagctgagtc gaggccagac cccctttgct gggacacagg acaagagcag ccttgtgggg
  9541 ttgcccccaa gcaagctgag tggccccatc ctggggccag ggtccttccc tagcgatgac
  9601 cgactctccc ggccacctcc accagccacg ccttcctcta tggatgtgaa cagccggcaa
  9661 ctggtaggag gctcccaagc tttctatcag cgagcaccct atcctgggtc cctgccctta
  9721 cagcagcaac agcaacaact gtggcagcaa caacaggcaa cagcagcaac ctccatgcga
  9781 tttgccatgt cagctcgctt tccatcaact cctggacctg aacttggccg ccaagcccta
  9841 ggttccccgt tggcgggaat ttccacccgt ctgccaggcc ctggtgagcc agtgcctggt
  9901 ccagctggtc ctgcccagtt cattgagctg cggcacaatg tacagaaagg actgggacct
  9961 gggggcactc cgtttcctgg tcagggccca cctcagagac cccgttttta ccctgtaagt
10021 gaggaccccc accgactggc tcctgaaggg cttcggggcc tggcggtatc aggtcttccc
10081 ccacagaaac cctcagcccc accggcccct gaattgaaca acagtcttca tccaacaccc
10141 cacaccaagg gtcctaccct gccaactggt ttggagctgg tcaaccggcc cccgtcgagc
10201 actgagcttg gccgccccaa tcctctggcc ctggaagctg ggaagttgcc ctgtgaggat
10261 cccgagctgg atgacgattt tgatgcccac aaggccctag aggatgatga agagcttgct
10321 cacctgggtc tgggtgtgga tgtggccaag ggtgatgatg aacttggcac cttagaaaac
10381 ctggagacca atgaccccca cttggatgac ctgctcaatg gagacgagtt tgacctgctg
10441 gcatatactg atcctgagct ggacactggg gacaagaagg atatcttcaa tgagcacctg
10501 aggctggtag aatcggctaa tgagaaggct gaacgggagg ccctgctgcg gggggtggag
10561 ccaggaccct tgggccctga ggagcgccct ccccctgctg ctgatgcctc tgaaccccgc
10621 ctggcatctg tgctccctga ggtgaagccc aaggtggagg agggtggacg ccacccttct
10681 ccttgccaat tcaccattgc tacccccaag gtagagcccg cacctgctgc caattccctt
10741 ggcctggggc taaagccagg acagagcatg atgggcagcc gggatacccg gatgggcaca
10801 gggccatttt ctagcagtgg gcacacagct gagaaggcct cctttggggc cacgggagga
10861 ccaccagctc acctgctgac ccccagccca ctgagtggcc caggaggatc ctccctgctg
10921 gaaaagtttg agctcgagag tggggctttg accttgcctg gtggacctgc agcatctggg
10981 gatgagctag acaagatgga gagctcactg gtagccagcg agttacccct gctcattgag
11041 gacctgttgg agcatgagaa gaaggagctg cagaagaagc agcagctttc agcacagttg
11101 cagcctgccc agcagcagca gcaacagcag cagcagcatt ccctactgtc tgcaccaggc
11161 cctgcccagg ccatgtcttt gccacatgag ggctcttctc ccagtttggc tgggtcccaa
11221 cagcagcttt ccctgggtct tgcaggtgcc cgacagccag gtttgcccca gccactgatg
11281 cccacccagc caccagctca tgccctccag caacgcctgg ctccatccat ggctatggtg
11341 tccaatcaag ggcatatgct aagtgggcag catggagggc aggcaggctt ggtaccccag
11401 cagagctcac agccagtgct atcacagaag cccatgggca ccatgccacc ttccatgtgc
11461 atgaagccgc agcaattggc aatgcagcag cagctggcaa acagcttctt cccagataca
11521 gacctggaca aatttgctgc agaagatatc attgatccca ttgcaaaggc caagatggtg
11581 gctttgaaag gcatcaagaa agtgatggct cagggcagca ttggggtggc acctggtatg
11641 aacagacagc aagtgtctct gctagcccag aggctctcgg ggggacctag cagtgatctg
11701 cagaaccatg tggcagctgg gagtggccag gagcggagtg ctggtgatcc ctcccagcct
11761 cgtcccaacc cgcccacttt tgctcaggga gtgatcaatg aagctgacca gcggcagtat
11821 gaggagtggc tgttccatac ccagcagctc ctacagatgc agctgaaggt gctagaggag
11881 cagattggtg tacaccgcaa gtcccggaag gctctgtgtg ccaagcagcg cactgccaaa
11941 aaagctggcc gtgagttccc agaagctgat gctgagaagc tcaagctggt tacagagcag
12001 cagagcaaga tccagaaaca actggatcag gtccggaaac agcagaagga gcacactaat
12061 ctcatggcag aatatcggaa caagcagcag caacaacagc agcagcagca gcaacaacag
12121 caacagcact cagctgtgct ggctctcagc ccttcccaga gtccccggct gctcaccaag
12181 ctccctggtc agctgctccc tggccatggg ctgcagccac cacaggggcc tccgggggg
12241 caagccggag gtcttcgcct gacccctggg ggtatggcac tacctggaca gcctggtggc
12301 cccttcctta atacagctct ggcccaacag cagcaacagc aacattctgg tggggctgga
12361 tccctggctg gcccttcagg gggcttcttc cctggcaacc ttgctcttcg aagcctcgga
12421 cctgattcaa ggcttttaca ggaaaggcag ctgcagctgc agcagcaacg tatgcagctg
12481 gcccagaaac tgcagcagca gcagcagcag caacagcagc agcagcacct tctaggacag
12541 gtggcaatcc agcagcaaca gcagcagggt cctggagtac agacaaacca agctctgggt
12601 cccaagcccc agggccttat gcctcccagc agccaccaag gcctcctggt ccagcagctg
12661 tcccctcaac caccccaggg gccccagggc atgctgggcc ctgcccaggt ggctgtgttg
12721 cagcagcagc accctggagc tttgggcccc cagggccctc acagacaggt gcttatgacc
12781 cagtcccggg tgctcagttc cccccagctg gcacagcagg gtcagggcct tatgggacac
12841 aggctggtca cagcccagca gcagcagcag caacaacagc accaacagca agggtccatg
12901 gcagggctgt cccatcttca gcagagtctg atgtcacaca gtgggcagcc caaactgagc
12961 gctcagccca tgggctcttt acagcagctt cagcagcagc agcagctgca acagcaacag
13021 caacttcagc agcagcagca gcagcagcta caacagcaac agcaacttca gcagcaacag
13081 cttcaacagc agcaacagca gcagcagctt caacaacagc agcagcaaca gcttcaacag
13141 cagcaacagc agctacaaca gcaacagcaa caacaacagc agcagtttca acagcagcag
13201 caacagcagc agatgggcct tttaaaccag agtcgaactt tactgtctcc tcagcaacaa
13261 cagcagcagc aagtggcact tggccctggc atgccagcaa agcctcttca acacttttct
13321 agccctggag ccctgggtcc aaccctcctc ctgacgggca aggaacaaaa caccgtagac
13381 ccagccgttt cttcagaggc cactgagggg ccctctacac atcagggagg gccgttagca
13441 ataggaacta cccctgagtc aatggccact gaaccaggag aggtaaagcc ctcactctct
13501 ggggactcac aactcctgct tgtccaaccc cagccccagc ctcagcccag ctctctgcag
13561 ctgcagccac ctctgaggct tccaggacaa cagcagcagc aagttagcct gctccacaca
13621 gcaggtggag gaagccatgg gcagctaggc agtggatcat cttctgaggc ctcatctgtg
13681 ccccacctgc tggctcagcc ctctgtttcc ttaggggatc agcctgggtc catgacccag
13741 aaccttctgg gcccccaaca gcccatgcta gagcggccca tgcaaaataa tacagggcca
13801 caacctccca aaccaggacc tgtcctccag tctgggcagg gtctgcctgg ggttggaatc
13861 atgcctacgg tgggtcagct tcgagcacag ctccaaggag tcctggccaa aaacccacag
13921 ctgcggcact taagtcctca gcagcagcag cagctacagg cactcctcat gcagcggcag
13981 ctgcagcaga gtcaggcagt acgccagacc ccaccctacc aggagcctgg gacccagacc
14041 tctcccctcc agggcctcct gggctgccaa cctcaacttg ggggcttccc tggaccacag
14101 acaggccccc tccaggagct aggggcaggg cctcgacctc agggcccacc ccggctccct
14161 gccccaccag gagccttatc tacaggacca gtccttggcc ctgtccatcc cacacctcca
14221 ccatccagcc ctcaagagcc aaagagacct tcacaattac cttcccccag ctcccagctt
14281 cccactgagg cccagctccc tcccacccat ccagggaccc ccaaacctca ggggccaacc
14341 ttggagccgc ctcctgggag ggtctcacct gctgctgccc agcttgcaga taccttgttt
14401 agcaagggtc tgggaccttg ggatccccca gacaacctag cagaaaccca gaagccagag
14461 cagagcagcc tggtacctgg gcatctggac caggtgaatg gacaggtggt gcctgaggca
14521 tcccaactca gcatcaagca ggaacctcgg gaagagccat gtgccctggg agcccagtca
14581 gtgaagaggg aggccaatgg ggagccaata ggggcaccag gaaccagcaa ccacctcctg
14641 ctggcaggcc ctcgctcaga agctgggcat ctgctcttgc agaagctact ccgggcaaag
14701 aatgtgcaac tcagcactgg gcgggggtcc gaggggctgc gagctgagat caacgggcac
14761 attgacagca agctggctgg gctggagcag aaactacagg gtacccccag caacaaggag
14821 gatgcagcag caaggaagcc tttgacaccg aagcccaagc gggtacagaa ggcaagcgac
14881 aggttggtga gctcccgaaa gaagctgcgg aaggaggacg gggtcagggc cagcgaggcc
14941 ttgctgaaac agctgaaaca ggagctgtcc ctgctgcccc taacggagcc tgctatcacc
15001 gccaatttta gcctctttgc cccctttggc agtggctgcc cagtcaatgg gcagagccag
15061 ctgagggggg cctttggaag tggggcgctg cccactggcc ctgactacta ttcccagctg
15121 cttaccaaga ataacctgag taacccgccg acaccaccct cgtcgctgcc ccccacccca
15181 cccccatcgg tgcagcagaa gatggtgaat ggcgtcaccc catctgaaga gctgggggag
15241 caccccaagg atgctgcctc tgcccgggat agtgaaaggg cactgaggga tacttcagag
15301 gtgaagagtc tagacctgct ggctgccttg cctacacccc ctcacaatca gactgaggat
15361 gtcaggatgg agagtgatga ggatagcgat tctcctgaca gcattgtgcc agcttcatcc
15421 cctgagagca tcttggggga ggaggcccct cgtttccctc atctgggctc aggccggtgg
15481 gagcaagagg accgggccct ctcccctgtc atccccctca ttcctcgggc cagcatccca
15541 gtcttcccag ataccaaacc ttatggggcc cttggcctgg aggtccctgg aaagctgcct
15601 gtcacaactt gggaaaaggg caaaggaagt gaggtgtcag tcatgctcac agtctctgct
15661 gctgcagcca agaacctgaa tggcgtgatg gtggcagtgg cggagctgct gagcatgaag
15721 atccccaact cctatgaggt gctgttccca gagagcccc cccgggcagg cactgagcca
15781 aagaaggggg aagctgaggg tcctggtggg aaggaaaagg gtctggaagg caagagccca
15841 gacactggcc ctgattggct gaagcagttt gatgcagtgt tgcctggcta taccctgaag
15901 agccaactag acatcttgag cctcctgaaa caggagagcc ccgccccaga gccacccact
15961 cagcacagct atacctacaa tgtctccaat ctggatgtgc gacagctctc ggccccacct
16021 cctgaagaac cctccccgcc cccttccccc ttggcacctt ctcctgccag tccccctact
16081 gagcccttgg ttgaacttcc caccgaaccc ttggctgagc cacccgtccc ctcacctctg
16141 ccactggcct catcccctga atcagcccga cccaagcccc gtgcccggcc ccctgaagaa
16201 ggtgaagatt cccgtcctcc tcgcctcaag aaatggaaag gagtgcgctg gaagcggctt
16261 cggctgctgc tgaccatcca gaagggcagt gggcggcagg aggatgagcg ggaagtggca
16321 gagtttatgg agcagcttgg cacagccttg cgacctgaca aggtaccgcg agacatgcgt
16381 cgctgctgtt tctgtcatga ggagggtgac ggggccactg atgggcctgc ccgtctgctg
16441 aacctggacc tggacctgtg ggtgcacctc aactgtgccc tttggtccac ggaggtgtat
16501 gagacccagg gcggggcact gatgaatgtg gaggttgccc tgcaccgagg actgctaacc
16561 aagtgctccc tgtgccagcg aactggtgcc accagcagct gcaatcgcat gcgttgcccc
16621 aatgtctacc attttgcttg tgccatccgt gccaagtgca tgttcttcaa ggacaagacc
16681 atgctgtgtc caatgcataa gatcaagggg ccctgtgagc aagagctgag ctcttttgct
16741 gtcttccgg gggtctacat tgagcgggac gaggtgaagc aaatcgctag catcattcag
16801 cggggagaac ggctgcacat gttccgtgtg gggggccttg tgttccacgc catcggacag
16861 ctgctgcctc accagatggc tgactttcat agtgccactg ccctctatcc cgtgggctac
16921 gaggccacgc gcatctattg gagcctccgc accaacaatc gtcgctgctg ctatcgctgt
16981 tctattggtg agaacaacgg gcggccggag tttgtaatca aagtcatcga gcagggcctg
17041 gaggacctgg tcttcactga cgcctctccc caggccgtgt ggaatcgcat cattgagcct
17101 gtggctgcca tgagaaaaga ggctgacatg ctgcgactct tccctgagta tctgaagggc
17161 gaggagctct ttgggctgac ggtgcatgcc gtgcttcgca tagctgaatc actgcccggg
17221 gtggagagct gtcaaaacta tttattccgc tatgggcgcc acccccttat ggagctgcca
17281 ctcatgatca accccactgg ctgtgcccga tcagagccta aaatcctcac acactacaaa
17341 cggccccata ccctgaacag caccagcatg tctaaggcat atcagagcac cttcacaggc
17401 gagaccaaca ccccctacag caagcagttt gtgcactcca agtcatctca gtaccggcgg
17461 ctgcgcaccg aatggaagaa caacgtgtac ctggctcgct cccgtatcca gggcctgggg
17521 ctctatgcag ccaaggacct agaaaagcac acaatggtta tcgagtacat tggcaccatc
17581 attcggaacg aggtggccaa ccggcgggag aaaatctacg aagagcagaa tcgaggcatc
17641 tacatgttcc gaataaacaa tgaacatgtg attgatgcta cgttgaccgg cggccctgcc
17701 aggtacatta accattcctg tgcccctaac tgtgtggccg aagtcgtgac atttgacaaa
17761 gaggacaaaa tcatcatcat ctccagccgg cgaatcccca aaggagagga gctaacctat
17821 gactatcagt ttgattttga ggacgatcag cacaagatcc cctgccactg tggagcctgg
17881 aattgtcgga aatggatgaa ctaagaagct ttgaggctac caggcagggg agtcccccta
17941 cccacaacct cttccctgaa agggatgagg gggaagagag gtagcagcca gagccaggac
18001 ccagggttgg ggctgccggc tgacccggag cccctggagc aggaggctgg ggcagagggc
18061 cctaggccaa gcccaccctg ggcaccaggg acaatcctct tccccaccac cggccctcag
18121 gctggcatct ctgcccccag ctccaggagg ggccagacag aagcagccat tgggcatctc
18181 aggtttgagg gggatatggg ccgggaacta cccagaagca tctgggaggc agcagggtgg
18241 gggaagagga tgtgtggccg ggcctcacag ccctgctgct cccactgacc tctccggccc
18301 aactcacggc tgcaaagaga cttgactaag cttgacaatc ccaaaggccg ggtcccacac
18361 ctggccctgc ctgccgggtc ctgcccccac cctcaccccc atccccctcc ctcttgatct
18421 gtctctgttt ccctcttttc ctctgtgttt ctgtctctct atgggttgtg tttccttgtt
18481 ttccactctg acaaatgcaa catgaacggg aaagaggcgc ccagctgcct aggagggcaa
18541 gctgggcaag ccgggcaagg agaccccgca cccacaccta cctcatttaa gtgttggatt
18601 ttttgctgtt ttgaaatgtg agaccctctc caagccccct actgccccaa ccctctcccc
18661 cacctcactg ccctcttctg agtgggtgga aggggggtag gaggaggaag aaaaacaaca
18721 acaaaaaatc catctttgtt tttaattatg ggcatgggat ggtggttgag gcaaatgatg
18781 atgaagattg gggatgactg gcccctagtt gctctaggac ttccttctcc atctggacat
18841 gggggcagga gggagctaaa cctaggacca ggatatctcc ctcctgtttt cccaacctca
18901 tcatgagcct gtttgccctc cagcccctgg acgggttggg tggggggtag ggtgagggct
18961 atccctgagt ggcatgccca tacctagtga ggcagggtgt ggcccggagc tcccactttc
19021 cctcagtcac caaactgctg ctggtctggt gggaaggggt ggtgatgtgg gggtggggga
19081 gcttagtgtc agcgcgggga gggtgggggg tatttatcta tttatacatg ggattgtaca
19141 tagtcttgtg gggcatgggg gagccggctg gaggtgagaa ccctcccctc tccccccacc
19201 ccccggggag agcaaatgta aaactactaa tttttgtgct ttatatattc tatataaata
19261 tatctatttt ctttttacaa aaccagttta taaatggtag gggggtgtgg ggcggacaca
19321 tggagctccc cttgtggggg ggccccctcc attacccgac ctaccgccct tttcctcacc
19381 ccccacccca ctccccaccc cctggctgtg actgctgtaa gatgggggta tagaggctgg
19441 gcaattccca ccccctgttg tatagttgga ctatgttata acgcacaaaa gagagctgac
19501 cccaggggga gccagagggt gatgggttcc ttgcctccct ttccttcccc tttctgccca
19561 agcttgtgct gcagttgaac ctcttcctgg gggtgggagt aggtaagggg tgggtgaggc
19621 cccaaacccc tctctggtag ggaaccgtgg ggatgaagat gaagcttata tgcagttctc
19681 ttctaggggc tgtgggcaaa gggcattttg taattaatat tttcaagaat cagatgtctg
19741 gagtgtaggg gtgggcttgg tggtggtgga cggggggcc tgctggaggg ggagcttggt
19801 cgctgttgtg attttaggtt tgtttttgtt ttttttgaa tttggggggt tgtggattgt
19861 tgggggtagg gagatttttt ttttttaaag ctgcttcctc aactgtttca agctgcaaat
19921 gtttaagaga ataacagccc ccactcccac aggaaccgct gtaattaaat cagacagtag
19981 gaagactggg ctgctgccct caaagccaca gcccttggat gttccttttc cgagagcaga
20041 aggtctaggc tacagggagg gggagattgg ctcccgtgag tcaggctgtg tttggggctt
20101 gggccctggg attgggaaaa ggggatgggg cagactttgt aagcatatgc taggtatccg
20161 atagtcctgt agaatttagt gaagaaacct tatacagttt ttaattttta tataaactat
20221 aactcagacc caagctacaa ggttggaatt ttggttggtt ttttttttaa gtaccctgcc
20281 tgtataattg catcagaatc ccccacccca ccccccgccc ccgtgtttgt attttgggtt
20341 ggtttacact cgcacatact cagttttcag ttttcccctt tacagtcttc tcccctcacc
20401 tccaggaccc tccccctttt taaaaaataa atcgctgaca agtgtgaatc ccgtgaagac
20461 tttattttgt gttgtgtgta tcctgtacag caaggttggt ccttcgtaac aacggatgaa
20521 atggttccct tttttaaagc gccctctctc cctccaccct cagcgcccct gtccttggca
20581 tgttttgtat cagcgatcat tctgaactgt acatatttat gttgcgagag gcaaagggca
20641 agttttggat tttgcttctt ccaagtttgt ttttaaacga caaataaaaa aagaacattt
20701 taaatacaa
KMT2B (accession No. NM_014727):
(SEQ ID NO: 135)
        1 atggcggcgg cggcgggcgg cggcagttgc cccgggcctg gctccgcgcg gggccgcttc
      61 ccgggccggc cgcggggcgc cggcgggggc gggggccgcg gcggacgggg caacggggcc
    121 gaaagagtgc gggtagctct gcggcgcggc ggtggcgcga cggggccggg cggagccgag
    181 cccggggagg acacggccct gctccgtttg ctggggctcc gccggggcct gcgccggctc
    241 cgccgcctgt gggccggccc ggggtccag cggggccggg gacggggtcg gggccggggc
    301 tggggcccga gtcgaggctg cgtgccggag gaggagagca gtgacgggga atccgacgag
    361 gaggagtttc agggttttca ttcagatgaa gatgtggccc ccagttccct gcgctctgcg
    421 ctccgatccc agcgaggtcg agcgccccga ggtcggggtc gcaagcataa gacgaccccc
    481 cttcctcctc ctcgcctagc agatgtggct cctacccccc caaagacccc tgcccggaaa
    541 cggggtgagg aaggcacaga acggatggtg caggcactga ctgaacttct ccggcgggcc
    601 caggcacccc aagcaccccg gagccgggca tgtgagccct ccaccccccg gcggtctcgg
    661 ggacggcccc caggacggcc agcaggcccc tgcaggagga agcagcaagc agtagtggtg
    721 gcagaagcag ctgtgacaat ccccaaacct gagcccccac ctcctgtggt tccagtgaaa
    781 catcagactg gcagctggaa atgcaaggag gggcccggtc caggacctgg gacccccagg
    841 cgtggaggac agtcaagccg tggaggccgt ggaggcaggg gccgcggccg aggtggtggg
    901 ctcccctttg tgatcaagtt tgtttcaagg gccaaaaaag taaagatggg acaattgtcc
    961 ttgggactcg aatcaggtca aggtcaaggt caacatgagg aaagttggca ggatgtcccc
  1021 caaagaagag ttggatctgg acagggaggg agcccttgct ggaaaaagca ggaacagaag
  1081 ctggatgacg aggaagaaga gaagaaagaa gaagaagaaa aagacaagga gggagaagag
  1141 aaggaagaaa gagctgtagc tgaggagatg atgccagctg cggaaaagga agaggcaaag
  1201 ctgccaccac cgcctctgac tcctccagcc ccttcacctc ctccacccct cccaccccct
  1261 tcgacatctc ctccaccccc actctgccct ccaccaccac ccccagtgtc cccaccacct
  1321 ctaccatccc ctccaccgcc tcctgcccaa gaggagcagg aggaatcccc tcctcctgtg
  1381 gtcccagcta cgtgctccag gaagaggggc cggcctcccc tgactcccag ccagcgggcg
  1441 gagcgggaag ctgctcgggc agggccagag ggcacctctc ctcccactcc aacccccagc
  1501 accgccacgg gaggccctcc ggaagacagt cccaccgtgg cccccaaaag caccaccttc
  1561 ctgaagaata tccggcagtt tattatgcct gtggtgagtg cccgctcctc ccgtgtcatc
  1621 aagacacccc ggcgatttat ggatgaagac ccccccaaac ccccaaaggt ggaggtctca
  1681 cctgtcctgc gacctcccat taccacctcc ccacctgttc cccaggagcc agcaccagtc
  1741 ccctctccac cacgtgcccc aactcctcca tctaccccag ttccactccc tgagaagaga
  1801 cggtccatcc taagggaacc cacatttcgc tggacctcac tgacccggga gctgccccct
  1861 cctcccccag cccctccacc tcccccggcc ccctccccac cccctgctcc tgccacctcc
  1921 tcccggaggc ccctactcct tcgggcccct cagtttaccc caagcgaagc ccacctgaag
  1981 atctacgaat cggtgcttac tcctcctcct cttggggctc ctgaagcccc tgagccagag
  2041 cctcctcctg ccgatgactc tccagctgag cctgagcctc gggcagtggg ccgcaccaac
  2101 cacctcagcc tgcctcgatt cgcccctgtg gtcaccactc ctgttaaggc cgaggtgtcc
  2161 cctcacgggg ctccagctct gagcaacggg ccacagacac aggctcagct actgcagccc
  2221 ctgcaggcct ticaaaccca gctcctgccc caggcactac cgccaccaca gccacagctg
  2281 cagccaccgc cgtcaccaca gcagatgcct cccctggaaa aagcccggat tgcgggcgtg
  2341 ggttccttgc cgctgtctgg ggtagaggag aagatgttca gcctcctcaa gagagccaaa
  2401 gtgcagctat tcaagatcga tcagcagcag cagcagaagg tggcagcttc catgccgctg
  2461 agccctggag ggcagatgga ggaggtggcc ggggctgtca agcagatctc cgacagaggc
  2521 cctgtccggt ctgaagatga gtcggtggaa gctaagagag agcggccctc aggtcccgag
  2581 tcccctgtgc aaggtccccg catcaaacat gtctgccgtc atgctgctgt ggccctgggt
  2641 caggcccggg ccatggtgcc tgaagatgtc cctcgcctca gtgccctccc tctccgggat
  2701 cggcaggacc tcgccacaga ggatacatca tcggcgtccg agactgagag tgtcccgtca
  2761 cggtcccggc ggggaaaggt ggaggcagca ggccctgggg gagaatcaga gcccacaggt
  2821 tctggaggga ccctggccca cacaccccgg cgctcactgc cctcccatca cggcaagaag
  2881 atgcgcatgg ctcgatgtgg acactgtcgg ggctgcctac gtgtgcagga ctgtgggtcc
  2941 tgtgtcaact gcctagacaa gcccaagttt gggggcccta acaccaagaa gcagtgctgt
  3001 gtataccgga agtgtgacaa aatagaggct cggaagatgg aacgactggc taaaaaaggc
  3061 cggacgatag tgaagacgct gttgccctgg gattccgatg aatctcctga ggcctcccct
  3121 ggtcctccag gcccacgccg gggggggga gctggggggc cccgggagga ggtggtggcc
  3181 cacccagggc ccgaggagca ggactccctc ctgcagcgca agtcagctcg gcgctgcgtc
  3241 aaacagcgac cctcctatga tatcttcgag gattcggatg actcggagcc cgggggcccc
  3301 cctgctcctc ggcgtcggac cccccgagaa aatgagctgc cactgccaga acctgaggag
  3361 cagagccggc cccgcaaacc taccctgcag cctgtgttgc agctcaaggc ccgaaggcgc
  3421 ctggacaagg atgctttggc ccctggcccc tttgcttctt ttcccaatgg ctggactgga
  3481 aagcagaagt ctcccgatgg tgtgcaccgc gtccgtgtgg attttaagga ggattgtgat
  3541 ttagagaacg tgtggctgat ggggggcctg agtgtgctca cctctgtgcc agggggcccc
  3601 ccgatggtgt gcttgctgtg tgccagcaaa ggactccacg agctggtgtt ctgtcaagtc
  3661 tgctgtgacc cattccaccc attctgcctg gaggaggccg agcggcccct gccccagcat
  3721 cacgacacct ggtgctgccg tcgctgcaaa ttctgccacg tctgtggacg caaaggtcgt
  3781 ggatccaagc acctcctgga gtgcgagcgc tgccgccatg cataccaccc ggcctgtctg
  3841 gggcccagct atccaacccg ggccacgcgc aaacggcgcc actggatctg ttcagcctgt
  3901 gtgcgctgta agagctgtgg ggcaactcca ggcaagaact gggacgtcga gtggtctgga
  3961 gattacagcc tctgccccag gtgcacccag ctatatgaga aaggaaacta ctgcccgatc
  4021 tgtacacgct gctatgaaga caacgactat gagagcaaga tgatgcagtg cgcacagtgc
  4081 gatcactggg tgcatgccaa gtgcgagggg ctctcagatg aagactacga gatcctttca
  4141 ggactgccag actcggtgct gtacacctgc ggaccgtgtg ctggggcagc gcagccccgc
  4201 tggcgagagg ccctgagcgg ggccctccag gggggcctgc gccaggtgct ccagggcctg
  4261 ctgagctcca aggtggtggg cccactgctg ctctgcaccc agtgtgggcc agatgggaag
  4321 caactgcacc caggaccctg cggcctgcaa gctgtgagtc agcgcttcga ggatggccac
  4381 tacaagtctg tgcacagctt catggaggac atggtgggca tcctcatgcg gcactcggag
  4441 gagggagaga ccccggaccg ccgggctgga ggccagatga aggggctcct gctgaagctg
  4501 ctagaatctg cgttcggctg gttcgacgcc cacgacccca agtactggcg acggagtacc
  4561 cggctgccaa acggagtcct tcccaatgcg gtgttgcccc catccctgga tcatgtctat
  4621 gcgcagtgga gacagcagga accagagacc ccagaatcag ggcagcctcc aggggatccc
  4681 tcagcagcat tccagggcaa ggatccggct gccttctcac acctggagga cccccgtcag
  4741 tgtgcactct gcctcaaata cggggatgca gactccaagg aggcggggcg gctcttgtac
  4801 atcgggcaga acgagtggac acacgtcaac tgtgccatct ggtcggcgga agtcttcgag
  4861 gagaacgacg gctccctcaa gaatgtgcat gctgctgtgg cccgagggag gcagatgcgc
  4921 tgcgagctct gcctgaagcc tggcgccacg gtgggctgct gcctgtcctc ctgcctcagc
  4981 aacttccact tcatgtgtgc ccgggccagc tactgcatct tccaggatga caagaaagtc
  5041 ttctgccaga aacacactga tctcctggat ggcaaggaaa ttgtgaaccc cgatggtttt
  5101 gatgttctcc gccgagtcta tgtggacttc gagggcatca acttcaagcg gaagttcttg
  5161 acggggcttg aacccgatgc catcaacgtg ctcattggtt ccatccgcat tgactccctg
  5221 ggtactctgt ctgatctctc ggactgcgag ggacggctct tccccattgg ctaccagtgc
  5281 tcccgtctgt actggagcac agtggatgct cggaggcgct gctggtatcg gtgccgaatt
  5341 ctggagtatc ggccatgggg gccgagggaa gagccagctc acctggaggc tgcagaggag
  5401 aaccagacca ttgtgcacag ccccgcccct tcctcagagc ccccaggtgg tgaggacccc
  5461 ccactggaca cagatgttct tgtccctgga gctcctgagc gccactcgcc cattcagaac
  5521 ctggaccctc cactgcggcc agattcaggc agcgcccctc ctccagcccc ccgttctttt
  5581 tcgggggctc gaatcaaagt gcccaactac tcgccatccc ggaggccctt ggggggtgtc
  5641 tcctttggcc ccctgccctc ccctggaagt ccatcttcac tgacccacca catccccaca
  5701 gtgggagacc cggacttccc agctcccccc agacgttccc gtcgtcccag ccctttggct
  5761 cccaggccgc ctccatcacg gtgggcctcc cctcctctaa aaacctcccc tcagctcagg
  5821 gtgccccctc ctacctcagt cgtcacagcc ctcacaccta cctcagggga gctggctccc
  5881 cctggcccgg ccccatctcc accaccccct gaagacctgg gcccagactt cgaggacatg
  5941 gaggtggtgt caggactgag tgctgctgac ctggacttcg cggccagcct gctggggact
  6001 gagcccttcc aggaagagat tgtagccgct ggggccatgg ggagcagcca cgggggcccg
  6061 ggggacagct ccgaggagga gtccagcccc acctcccgct acatccactt ccctgtgact
  6121 gtggtgtccg cccctggtct ggcccccagc gctacccctg gagccccccg cattgaacag
  6181 ctggacggcg tggacgacgg cactgacagt gaggctgagg cggtgcagca gcctcggggc
  6241 cagggcacgc ctccttcggg gccaggagta gtccgggcag gggtccttgg ggctgcaggg
  6301 gacagggccc ggcctcctga ggacctgcca tcggaaattg tggattttgt gttgaagaac
  6361 ctagggggtc ctggggatgg aggtgctggc cctagagagg agtcactccc cccggcgcct
  6421 cccctggcta atggcagcca gccctcccaa ggcctgaccg ccagcccagc tgaccccacc
  6481 cgcacatttg cctggctccc aggggcccca ggggtccggg tgttaagcct tggccctgcc
  6541 cctgagcccc ccaaacccgc cacatccaaa atcatacttg tcaacaagct ggggcaagta
  6601 tttgtgaaga tggctgggga gggtgaacct gtcccacccc cagtgaagca gccacctttg
  6661 ccccccacca tttcccccac ggctcccacc tcctggactc tgcccccagg ccccctcctc
  6721 ggcgtgctgc ccgtggtcgg agtggtccgc cctgccccgc ccccgccacc ccctcccctg
  6781 acgctggtgc tgagcagtgg gccagccagc ccgccccgcc aggccatccg cgtcaagagg
  6841 gtgtccactt tctccggccg gtccccgcca gcacctcccc catacaaagc cccccggctg
  6901 gatgaagatg gagaggcctc agaggatacc cctcaggttc cagggcttgg cagtggcggg
  6961 tttagccgtg tgaggatgaa aacccccaca gtgcgtgggg tccttgacct ggatcggcct
  7021 ggggagcccg ctggggaaga aagtcctggg cccctccagg aacggtcccc tttgctgcca
  7081 cttccggaag atggtcctcc ccaggtcccc gatggtcccc cagacctgct gcttgagtcc
  7141 cagtggcacc actattcagg tgaggcttcg agctctgagg aagagcctcc atccccagat
  7201 gataaagaga accaggcccc aaaacggact ggcccacatc tgcgcttcga gatcagcagt
  7261 gaggatgggt tcagcgttga ggcagagagc ttggaggggg cgtggagaac tctgatcgag
  7321 aaagtgcaag aggcccgagg gcatgcccga ctcagacatc tctcctttag tggaatgagt
  7381 ggggcgagac tcctgggcat ccaccatgat gctgtcatct tcctggccga gcagctcccc
  7441 ggagcccagc gttgccagca ctataagttc cgttaccacc agcagggaga gggccaggag
  7501 gagccgcccc tgaatcccca tggggctgct cgggcagagg tctatctccg gaagtgcacc
  7561 tttgacatgt tcaacttcct ggcctcccag caccgggtgc tccctgaggg ggccacctgt
  7621 gatgaggaag aggatgaggt gcagctcagg tcaaccagac gtgccaccag cctggagctg
  7681 cccatggcca tgcgttttcg tcaccttaag aagacgtcca aagaagctgt gggtgtctac
  7741 agatcagcca tccacgggcg aggcctgttc tgtaagcgca acatcgacgc gggggagatg
  7801 gtcatcgagt actctggcat tgtcatccgc tcggtgttga ctgacaagcg ggagaagttc
  7861 tacgatggga agggcatcgg gtgctatatg ttccgcatgg atgactttga tgtagtggac
  7921 gccacgatgc atggcaatgc cgcccgcttc atcaaccact cctgtgagcc caactgcttc
  7981 tctcgggtca tccacgtgga gggccagaaa cacattgtta tcttcgccct gcgccgcatc
  8041 ctgcgtggtg aggagctcac ctacgactac aagttcccca tcgaggatgc cagcaacaag
  8101 ctgccctgca actgtggcgc caagcgctgc cgtcggttcc ttaactgagg ccgtggctgc
  8161 ccaccacgac ccctcacacc tcctgctgcc gtcgctgcca tcttgcccct agcctggggg
  8221 ctccctagcc cctcccagag catctcaccc ccaccctcat gttcagggtg gatgtgggca
  8281 tgcaggtgac aagggccctg cctccacccc tccagcccat ccagcaatcg ccccctttct
  8341 gccctggggg cccaggatgt agatattgta caaaggtttc taaatccctt cttttctatg
  8401 cactttttta tttaagaggt ggggtcccag gtgggaaccc ccccacaata aagtctgtca
  8461 atgtttggag aaaaaaaaaa aaaaaaaaaa
KMTC (accession No. NM_170606)
(SEQ ID NO: 136)
        1 gaggtgcgcg cgcccgcgcc gatgtgtgtg agtgcgtgtc ctgctcgctc catgttgccg
      61 cctctcccgg tacctgctgc tgctcccggg gctgcgggaa atgcgagagg ctgagccggg
    121 gaggaggaac ccgagcagca gcggcggcgg cggcggccgc ggcggcggga gccccccagg
    181 aggaggaccg ggatccatgt gtctttcctg gtgactagga tgtcgtcgga ggaggacaag
    241 agcgtggagc agccgcagcc gccgccacca ccccccgagg agcctggagc cccggccccg
    301 agccccgcag ccgcagacaa aagacctcgg ggccggcctc gcaaagatgg cgcttcccct
    361 ttccagagag ccagaaagaa acctcgaagt agggggaaaa ctgcagtgga agatgaggac
    421 agcatggatg ggctggagac aacagaaaca gaaacgattg tggaaacaga aatcaaagaa
    481 caatctgcag aagaggatgc tgaagcagaa gtggataaca gcaaacagct aattccaact
    541 cttcagcgat ctgtgtctga ggaatcggca aactccctgg tctctgttgg tgtagaagcc
    601 aaaatcagtg aacagctctg cgctttttgt tactgtgggg aaaaaagttc cttaggacaa
    661 ggagacttaa aacaattcag aataacgcct ggatttatct tgccatggag aaaccaacct
    721 tctaacaaga aggacattga tgacaacagc aatggaacct atgagaaaat gcaaaactca
    781 gcaccacgaa aacaaagagg acagagaaaa gaacgatctc ctcagcagaa tatagtatct
    841 tgtgtaagtg taagcaccca gacagcttca gatgatcaag ctggtaaact gtgggatgaa
    901 ctcagtctgg ttgggcttcc agatgccatt gatatccaag ccttatttga ttctacaggc
    961 acttgttggg ctcatcaccg ttgtgtggag tggtcactag gagtatgcca gatggaagaa
  1021 ccattgttag tgaacgtgga caaagctgtt gtctcaggga gcacagaacg atgtgcattt
  1081 tgtaagcacc ttggagccac tatcaaatgc tgtgaagaga aatgtaccca gatgtatcat
  1141 tatccttgtg ctgcaggagc cggcaccttt caggatttca gtcacatctt cctgctttgt
  1201 ccagaacaca ttgaccaagc tcctgaaaga tcgaaggaag atgcaaactg tgcagtgtgc
  1261 gacagcccgg gagacctctt agatcagttc ttttgtacta cttgtggtca gcactatcat
  1321 ggaatgtgcc tggatatagc ggttactcca ttaaaacgtg caggttggca atgtcctgag
  1381 tgcaaagtgt gccagaactg caaacaatcg ggagaagata gcaagatgct agtgtgtgat
  1441 acgtgtgaca aagggtatca tactttttgt cttcaaccag ttatgaaatc agtaccaacc
  1501 aatggctgga aatgcaaaaa ttgcagaata tgtatagagt gtggcacacg gtctagttct
  1561 cagtggcacc acaattgcct gatatgtgac aattgttacc aacagcagga taacttatgt
  1621 cccttctgtg ggaagtgtta tcatccagaa ttgcagaaag acatgcttca ttgtaatatg
  1681 tgcaaaaggt gggttcacct agagtgtgac aaaccaacag atcatgaact ggatactcag
  1741 ctcaaagaag agtatatctg catgtattgt aaacacctgg gagctgagat ggatcgttta
  1801 cagccaggtg aggaagtgga gatagctgag ctcactacag attataacaa tgaaatggaa
  1861 gttgaaggcc ctgaagatca aatggtattc tcagagcagg cagctaataa agatgtcaac
  1921 ggtcaggagt ccactcctgg aattgttcca gatgcggttc aagtccacac tgaagagcaa
  1981 cagaagagtc atccctcaga aagtcttgac acagatagtc ttcttattgc tgtatcatcc
  2041 caacatacag tgaatactga attggaaaaa cagatttcta atgaagttga tagtgaagac
  2101 ctgaaaatgt cttctgaagt gaagcatatt tgtggcgaag atcaaattga agataaaatg
  2161 gaagtgacag aaaacattga agtcgttaca caccagatca ctgtgcagca agaacaactg
  2221 cagttgttag aggaacctga aacagtggta tccagagaag aatcaaggcc tccaaaatta
  2281 gtcatggaat ctgtcactct tccactagaa accttagtgt ccccacatga ggaaagtatt
  2341 tcattatgtc ctgaggaaca gttggttata gaaaggctac aaggagaaaa ggaacagaaa
  2401 gaaaattctg aactttctac tggattgatg gactctgaaa tgactcctac aattgagggt
  2461 tgtgtgaaag atgtttcata ccaaggaggc aaatctataa agttatcatc tgagacagag
  2521 tcatcatttt catcatcagc agacataagc aaggcagatg tgtcttcctc cccaacacct
  2581 tcttcagact tgccttcgca tgacatgctg cataattacc cttcagctct tagttcctct
  2641 gctggaaaca tcatgccaac aacttacatc tcagtcactc caaaaattgg catgggtaaa
  2701 ccagctatta ctaagagaaa attttctcct ggtagacctc ggtccaaaca gggggcttgg
  2761 agtacccata atacagtgag cccaccttcc tggtccccag acatttcaga aggtcgggaa
  2821 atttttaaac ccaggcagct tcctggcagt gccatttgga gcatcaaagt gggccgtggg
  2881 tctggatttc caggaaagcg gagacctcga ggtgcaggac tgtcggggcg aggtggccga
  2941 ggcaggtcaa agctgaaaag tggaatcgga gctgttgtat tacctggggt gtctactgca
  3001 gatatttcat caaataagga tgatgaagaa aactctatgc acaatacagt tgtgttgttt
  3061 tctagcagtg acaagttcac tttgaatcag gatatgtgtg tagtttgtgg cagttttggc
  3121 caaggagcag aaggaagatt acttgcctgt tctcagtgtg gtcagtgtta ccatccatac
  3181 tgtgtcagta ttaagatcac taaagtggtt cttagcaaag gttggaggtg tcttgagtgc
  3241 actgtgtgtg aggcctgtgg gaaggcaact gacccaggaa gactcctgct gtgtgatgac
  3301 tgtgacataa gttatcacac ctactgccta gaccctccat tgcagacagt tcccaaagga
  3361 ggctggaagt gcaaatggtg tgtttggtgc agacactgtg gagcaacatc tgcaggtcta
  3421 agatgtgaat ggcagaacaa ttacacacag tgcgctcctt gtgcaagctt atcttcctgt
  3481 ccagtctgct atcgaaacta tagagaagaa gatcttattc tgcaatgtag acaatgtgat
  3541 agatggatgc atgcagtttg tcagaactta aatactgagg aagaagtgga aaatgtagca
  3601 gacattggtt ttgattgtag catgtgcaga ccctatatgc ctgcgtctaa tgtgccttcc
  3661 tcagactgct gtgaatcttc acttgtagca caaattgtca caaaagtaaa agagctagac
  3721 ccacccaaga cttataccca ggatggtgtg tgtttgactg aatcagggat gactcagtta
  3781 cagagcctca cagttacagt tccaagaaga aaacggtcaa aaccaaaatt gaaattgaag
  3841 attataaatc agaatagcgt ggccgtcctt cagacccctc cagacatcca atcagagcat
  3901 tcaagggatg gtgaaatgga tgatagtcga gaaggagaac ttatggattg tgatggaaaa
  3961 tcagaatcta gtcctgagcg ggaagctgtg gatgatgaaa ctaagggagt ggaaggaaca
  4021 gatggtgtca aaaagagaaa aaggaaacca tacagaccag gtattggtgg atttatggtg
  4081 cggcaaagaa gtcgaactgg gcaagggaaa accaaaagat ctgtgatcag aaaagattcc
  4141 tcaggctcta tttccgagca gttaccttgc agagatgatg gctggagtga gcagttacca
  4201 gatactttag ttgatgaatc tgtttctgtt actgaaagca ctgaaaaaat aaagaagaga
  4261 taccgaaaaa ggaaaaataa gcttgaagaa actttccctg cctatttaca agaagctttc
  4321 tttggaaaag atcttctaga tacaagtaga caaagcaaga taagtttaga taatctgtca
  4381 gaagatggag ctcagctttt atataaaaca aacatgaaca caggtttctt ggatccttcc
  4441 ttagatccac tacttagttc atcctcggct ccaacaaaat ctggaactca cggtcctgct
  4501 gatgacccat tagctgatat ttctgaagtt ttaaacacag atgatgacat tcttggaata
  4561 atttcagatg atctagcaaa atcagttgat cattcagata ttggtcctgt cactgatgat
  4621 ccttcctctt tgcctcagcc aaatgtcaat cagagttcac gaccattaag tgaagaacag
  4681 ctagatggga tcctcagtcc tgaactagac aaaatggtca cagatggagc aattcttgga
  4741 aaattatata aaattccaga gcttggcgga aaagatgttg aagacttatt tacagctgta
  4801 cttagtcctg cgaacactca gccaactcca ttgccacagc ctcccccacc aacacagctg
  4861 ttgccaatac acaatcagga tgctttttca cggatgcctc tcatgaatgg ccttattgga
  4921 tccagtcctc atctcccaca taattctttg ccacctggaa gcggactggg aactttctct
  4981 gcaattgcac aatcctctta tcctgatgcc agggataaaa attcagcctt taatccaatg
  5041 gcaagtgatc ctaacaactc ttggacatca tcagctccca ctgtggaagg agaaaatgac
  5101 acaatgtcga atgcccagag aagcacgctt aagtgggaga aagaggaggc tctgggtgaa
  5161 atggcaactg ttgccccagt tctctacacc aatattaatt tccccaactt aaaggaagaa
  5221 ttccctgatt ggactactag agtgaagcaa attgccaaat tgtggagaaa agcaagctca
  5281 caagaaagag caccatatgt gcaaaaagcc agagataaca gagctgcttt acgcattaat
  5341 aaagtacaga tgtcaaatga ttccatgaaa aggcagcaac agcaagatag cattgatccc
  5401 agctctcgta ttgattcgga gctttttaaa gatcctttaa agcaaagaga atcagaacat
  5461 gaacaggaat ggaaatttag acagcaaatg cgtcagaaaa gtaagcagca agctaaaatt
  5521 gaagccacac agaaacttga acaggtgaaa aatgagcagc agcagcagca acaacagcaa
  5581 tttggttctc agcatcttct ggtgcagtct ggttcagata caccaagtag tgggatacag
  5641 agtcccttga cacctcagcc tggcaatgga aatatgtctc ctgcacagtc attccataaa
  5701 gaactgttta caaaacagcc acccagtacc cctacgtcta catcttcaga tgatgtgttt
  5761 gtaaagccac aagctccacc tcctcctcca gccccatccc ggattcccat ccaggatagt
  5821 ctttctcagg ctcagacttc tcagccaccc tcaccgcaag tgttttcacc tgggtcctct
  5881 aactcacgac caccatctcc aatggatcca tatgcaaaaa tggttggtac ccctcgacca
  5941 cctcctgtgg gccatagttt ttccagaaga aattctgctg caccagtgga aaactgtaca
  6001 cctttatcat cggtatctag gccccttcaa atgaatgaga caacagcaaa taggccatcc
  6061 cctgtcagag atttatgttc ttcttccacg acaaataatg acccctatgc aaaacctcca
  6121 gacacaccta ggcctgtgat gacagatcaa tttcccaaat ccttgggcct atcccggtct
  6181 cctgtagttt cagaacaaac tgcaaaaggc cctatagcag ctggaaccag tgatcacttt
  6241 actaaaccat ctcctagggc agatgtgttt caaagacaaa ggatacctga ctcatatgca
  6301 cgacccttgt tgacacctgc acctcttgat agtggtcctg gaccttttaa gactccaatg
  6361 caacctcctc catcctctca ggatccttat ggatcagtgt cacaggcatc aaggcgattg
  6421 tctgttgacc cttatgaaag gcctgctttg acaccaagac ctatagataa tttttctcat
  6481 aatcagtcaa atgatccata tagtcagcct ccccttaccc cacatccagc agtgaatgaa
  6541 tcttttgccc atccttcaag ggctttttcc cagcctggaa ccatatcaag gccaacatct
  6601 caggacccat actcccaacc cccaggaact ccacgacctg ttgtagattc ttattcccaa
  6661 tcttcaggaa cagctaggtc caatacagac ccttactctc aacctcctgg aactccccgg
  6721 cctactactg ttgacccata tagtcagcag ccccaaaccc caagaccatc tacacaaact
  6781 gacttgtttg ttacacctgt aacaaatcag aggcattctg atccatatgc tcatcctcct
  6841 ggaacaccaa gacctggaat ttctgtccct tactctcagc caccagcaac accaaggcca
  6901 aggatttcag agggttttac taggtcctca atgacaagac cagtcctcat gccaaatcag
  6961 gatcctttcc tgcaagcagc acaaaaccga ggaccagctt tacctggccc gttggtaagg
  7021 ccacctgata catgttccca gacacctagg ccccctggac ctggtctttc agacacattt
  7081 agccgtgttt ccccatctgc tgcccgtgat ccctatgatc agtctccaat gactccaaga
  7141 tctcagtctg actcttttgg aacaagtcaa actgcccatg atgttgctga tcagccaagg
  7201 cctggatcag aggggagctt ctgtgcatct tcaaactctc caatgcactc ccaaggccag
  7261 cagttctctg gtgtctccca acttcctgga cctgtgccaa cttcaggagt aactgataca
  7321 cagaatactg taaatatggc ccaagcagat acagagaaat tgagacagcg gcagaagtta
  7381 cgtgaaatca ttctccagca gcaacagcag aagaagattg caggtcgaca ggagaagggg
  7441 tcacaggact cacccgcagt gcctcatcca gggcctcttc aacactggca accagagaat
  7501 gttaaccagg ctttcaccag acccccacct ccctatcctg ggaacattag gtctcctgtt
  7561 gcccctcctt taggacctag atatgctgtt ttcccaaaag atcagcgtgg accctatcct
  7621 cctgatgttg ctagtatggg gatgagacct catggattta gatttggatt tccaggaggt
  7681 agtcatggta ccatgccgag tcaagagcgc ttccttgtgc ctcctcagca aatacaggga
  7741 tctggagttt ctccacagct aagaagatca gtatctgtag atatgcctag gcctttaaat
  7801 aactcacaaa tgaataatcc agttggactt cctcagcatt tttcaccaca gagcttgcca
  7861 gttcagcagc acaacatact gggccaagca tatattgaac tgagacatag ggctcctgac
  7921 ggaaggcaac ggctgccttt cagtgctcca cctggcagcg ttgtagaggc atcttctaat
  7981 ctgagacatg gaaacttcat tccccggcca gactttccgg gccctagaca cacagacccc
  8041 atgcgacgac ctccccaggg tctacctaat cagctacctg tgcacccaga tttggaacaa
  8101 gtgccaccat ctcaacaaga gcaaggtcat tctgtccatt catcttctat ggtcatgagg
  8161 actctgaacc atccactagg tggtgaattt tcagaagctc ctttgtcaac atctgtaccg
  8221 tctgaaacaa cgtctgataa tttacagata accacccagc cttctgatgg tctagaggaa
  8281 aaacttgatt ctgatgaccc ttctgtgaag gaactggatg ttaaagacct tgagggggtt
  8341 gaagtcaaag acttagatga tgaagatctt gaaaacttaa atttagatac agaggatggc
  8401 aaggtagttg aattggatac tttagataat ttggaaacta atgatcccaa cctggatgac
  8461 ctcttaaggt caggagagtt tgatatcatt gcatatacag atccagaact tgacatggga
  8521 gataagaaaa gcatgtttaa tgaggaacta gaccttccaa ttgatgataa gttagataat
  8581 cagtgtgtat ctgttgaacc aaaaaaaaag gaacaagaaa acaaaactct ggttctctct
  8641 gataaacatt caccacagaa aaaatccact gttaccaatg aggtaaaaac ggaagtactg
  8701 tctccaaatt ctaaggtgga atccaaatgt gaaactgaaa aaaatgatga gaataaagat
  8761 aatgttgaca ctccttgctc acaggcttct gctcactcag acctaaatga tggagaaaag
  8821 acttctttgc atccttgtga tccagatcta tttgagaaaa gaaccaatcg agaaactgct
  8881 ggccccagtg caaatgtcat tcaggcatcc actcaactac ctgctcaaga tgtaataaac
  8941 tcttgtggca taactggatc aactccagtt ctctcaagtt tacttgctaa tgagaaatct
  9001 gataattcag acattaggcc atcggggtct ccaccaccac caactctgcc ggcctcccca
  9061 tccaatcatg tgtcaagttt gcctcctttc atagcaccgc ctggccgtgt tttggataat
  9121 gccatgaatt ctaatgtgac agtagtctct agggtaaacc atgttttttc tcagggtgtg
  9181 caggtaaacc cagggctcat tccaggtcaa tcaacagtta accacagtct ggggacagga
  9241 aaacctgcaa ctcaaactgg gcctcaaaca agtcagtctg gtaccagtag catgtctgga
  9301 ccccaacagc taatgattcc tcaaacatta gcacagcaga atagagagag gccccttctt
  9361 ctagaagaac agcctctact tctacaggat cttttggatc aagaaaggca agaacagcag
  9421 cagcaaagac agatgcaagc catgattcgt cagcgatcag aaccgttctt ccctaatatt
  9481 gattttgatg caattacaga tcctataatg aaagccaaaa tggtggccct taaaggtata
  9541 aataaagtga tggcacaaaa caatctgggc atgccaccaa tggtgatgag caggttccct
  9601 tttatgggcc aggtggtaac tggaacacag aacagtgaag gacagaacct tggaccacag
  9661 gccattcctc aggatggcag tataacacat cagatttcta ggcctaatcc tccaaatttt
  9721 ggtccaggct ttgtcaatga ttcacagcgt aagcagtatg aagagtggct ccaggagacc
  9781 caacagctgc ttcaaatgca gcagaagtat cttgaagaac aaattggtgc tcacagaaaa
  9841 tctaagaagg ccctttcagc taaacaacgt actgccaaga aagctgggcg tgaatttcca
  9901 gaggaagatg cagaacaact caagcatgtt actgaacagc aaagcatggt tcagaaacag
  9961 ctagaacaga ttcgtaaaca acagaaagaa catgctgaat tgattgaaga ttatcggatc
10021 aaacagcagc agcaatgtgc aatggcccca cctaccatga tgcccagtgt ccagccccag
10081 ccacccctaa ttccaggtgc cactccaccc accatgagcc aacccacctt tcccatggtg
10141 ccacagcagc ttcagcacca gcagcacaca acagttattt ctggccatac tagccctgtt
10201 agaatgccca gtttacctgg atggcaaccc aacagtgctc ctgcccacct gcccctcaat
10261 cctcctagaa ttcagccccc aattgcccag ttaccaataa aaacttgtac accagcccca
10321 gggacagtct caaatgcaaa tccacagagt ggaccaccac ctcgggtaga atttgatgac
10381 aacaatccct ttagtgaaag ttttcaagaa cgggaacgta aggaacgttt acgagaacag
10441 caagagagac aacggatcca actcatgcag gaggtagata gacaaagagc tttgcagcag
10501 aggatggaaa tggagcagca tggtatggtg ggctctgaga taagtagtag taggacatct
10561 gtgtcccaga ttcccttcta cagttccgac ttaccttgtg attttatgca acctctagga
10621 ccccttcagc agtctccaca acaccaacag caaatggggc aggttttaca gcagcagaat
10681 atacaacaag gatcaattaa ttcaccctcc acccaaactt tcatgcagac taatgagcga
10741 aggcaggtag gccctccttc atttgttcct gattcaccat caatccctgt tggaagccca
10801 aatttttctt ctgtgaagca gggacatgga aatctttctg ggaccagctt ccagcagtcc
10861 ccagtgaggc cttcttttac acctgcttta ccagcagcac ctccagtagc taatagcagt
10921 ctcccatgtg gccaagattc tactataacc catggacaca gttatccggg atcaacccaa
10981 tcgctcattc agttgtattc tgatataatc ccagaggaaa aagggaaaaa gaaaagaaca
11041 agaaagaaga aaagagatga tgatgcagaa tccaccaagg ctccatcaac tccccattca
11101 gatataactg ccccaccgac tccaggcatc tcagaaacta cctctactcc tgcagtgagc
11161 acacccagtg agcttcctca acaagccgac caagagtcgg tggaaccagt cggcccatcc
11221 actcccaata tggcagcagg ccagctatgt acagaattag agaacaaact gcccaatagt
11281 gatttctcac aagcaactcc aaatcaacag acgtatgcaa attcagaagt agacaagctc
11341 tccatggaaa cccctgccaa aacagaagag ataaaactgg aaaaggctga gacagagtcc
11401 tgcccaggcc aagaggagcc taaattggag gaacagaatg gtagtaaggt agaaggaaac
11461 gctgtagcct gtcctgtctc ctcagcacag agtcctcccc attctgctgg ggcccctgct
11521 gccaaaggag actcagggaa tgaacttctg aaacacttgt tgaaaaataa aaagtcatct
11581 tctcttttga atcaaaaacc tgagggcagt atttgttcag aagatgactg tacaaaggat
11641 aataaactag ttgagaagca gaacccagct gaaggactgc aaactttggg ggctcaaatg
11701 caaggtggtt ttggatgtgg caaccagttg ccaaaaacag atggaggaag tgaaaccaag
11761 aaacagcgaa gcaaacggac tcagaggacg ggtgagaaag cagcacctcg ctcaaagaaa
11821 aggaaaaagg acgaagagga gaaacaagct atgtactcta gcactgacac gtttacccac
11881 ttgaaacagc agaataattt aagtaatcct ccaacacccc ctgcctctct tcctcctaca
11941 ccacctccta tggcttgtca gaagatggcc aatggttttg caacaactga agaacttgct
12001 ggaaaagccg gagtgttagt gagccatgaa gttaccaaaa ctctaggacc taaaccattt
12061 cagctgccct tcagacccca ggacgacttg ttggcccgag ctcttgctca gggccccaag
12121 acagttgatg tgccagcctc cctcccaaca ccacctcata acaatcagga agaattaagg
12181 atacaggatc actgtggtga tcgagatact cctgacagtt ttgttccctc atcctctcct
12241 gagagtgtgg ttggggtaga agtgagcagg tatccagatc tgtcattggt caaggaggag
12301 cctccagaac cggtgccgtc ccccatcatt ccaattcttc ctagcactgc tgggaaaagt
12361 tcagaatcaa gaaggaatga catcaaaact gagccaggca ctttatattt tgcgtcacct
12421 tttggtcctt ccccaaatgg tcccagatca ggtcttatat ctgtagcaat tactctgcat
12481 cctacagctg ctgagaacat tagcagtgtt gtggctgcat tttccgacct tcttcacgtc
12541 cgaatcccta acagctatga ggttagcagt gctccagatg tcccatccat gggtttggtc
12601 agtagccaca gaatcaaccc gggtttggag tatcgacagc atttacttct ccgtgggcct
12661 ccgccaggat ctgcaaaccc tcccagatta gtgagctctt accggctgaa gcagcctaat
12721 gtaccatttc ctccaacaag caatggtctt tctggatata aggattctag tcatggtatt
12781 gcagaaagcg cagcactcag accacagtgg tgttgtcatt gtaaagtggt tattcttgga
12841 agtggtgtgc ggaaatcttt caaagatctg acccttttga acaaggattc ccgagaaagc
12901 accaagaggg tagagaagga cattgtcttc tgtagtaata actgctttat tctttattca
12961 tcaactgcac aagcgaaaaa ctcagaaaac aaggaatcca ttccttcatt gccacaatca
13021 cctatgagag aaacgccttc caaagcattt catcagtaca gcaacaacat ctccactttg
13081 gatgtgcact gtctccccca gctcccagag aaagcttctc cccctgcctc accacccatc
13141 gccttccctc ctgcttttga agcagcccaa gtcgaggcca agccagatga gctgaaggtg
13201 acagtcaagc tgaagcctcg gctaagagct gtccatggtg ggtttgaaga ttgcaggccg
13261 ctcaataaaa aatggagagg aatgaaatgg aagaagtgga gcattcatat tgtaatccct
13321 aaggggacat ttaaaccacc ttgtgaggat gaaatagatg aatttctaaa gaaattgggc
13381 acttccctta aacctgatcc tgtgcccaaa gactatcgga aatgttgctt ttgtcatgaa
13441 gaaggtgatg gattgacaga tggaccagca aggctactca accttgactt ggatctgtgg
13501 gtccacttga actgcgctct gtggtccacg gaggtctatg agactcaggc tggtgcctta
13561 ataaatgtgg agctagctct gaggagaggc ctacaaatga aatgtgtctt ctgtcacaag
13621 acgggtgcca ctagtggatg ccacagattt cgatgcacca acatttatca cttcacttgc
13681 gccattaaag cacaatgcat gttttttaag gacaaaacta tgctttgccc catgcacaaa
13741 ccaaagggaa ttcatgagca agaattaagt tactttgcag tcttcaggag ggtctatgtt
13801 cagcgtgatg aggtgcgaca gattgctagc atcgtgcaac gaggagaacg ggaccatacc
13861 tttcgcgtgg gtagcctcat cttccacaca attggtcagc tgcttccaca gcagatgcaa
13921 gcattccatt ctcctaaagc actcttccct gtgggctatg aagccagccg gctgtactgg
13981 agcactcgct atgccaatag gcgctgccgc tacctgtgct ccattgagga gaaggatggg
14041 cgcccagtgt ttgtcatcag gattgtggaa caaggccatg aagacctggt tctaagtgac
14101 atctcaccta aaggtgtctg ggataagatt ttggagcctg tggcatgtgt gagaaaaaag
14161 tctgaaatgc tccagctttt cccagcgtat ttaaaaggag aggatctgtt tggcctgacc
14221 gtctctgcag tggcacgcat agcggaatca cttcctgggg ttgaggcatg tgaaaattat
14281 accttccgat acggccgaaa tcctctcatg gaacttcctc ttgccgttaa ccccacaggt
14341 tgtgcccgtt ctgaacctaa aatgagtgcc catgtcaaga ggtttgtgtt aaggcctcac
14401 accttaaaca gcaccagcac ctcaaagtca tttcagagca cagtcactgg agaactgaac
14461 gcaccttata gtaaacagtt tgttcactcc aagtcatcgc agtaccggaa gatgaaaact
14521 gaatggaaat ccaatgtgta tctggcacgg tctcggattc aggggctggg cctgtatgct
14581 gctcgagaca ttgagaaaca caccatggtc attgagtaca tcgggactat cattcgaaac
14641 gaagtagcca acaggaaaga gaagctttat gagtctcaga accgtggtgt gtacatgttc
14701 cgcatggata acgaccatgt gattgacgcg acgctcacag gagggcccgc aaggtatatc
14761 aaccattcgt gtgcacctaa ttgtgtggct gaagtggtga cttttgagag aggacacaaa
14821 attatcatca gctccagtcg gagaatccag aaaggagaag agctctgcta tgactataag
14881 tttgactttg aagatgacca gcacaagatt ccgtgtcact gtggagctgt gaactgccgg
14941 aagtggatga actgaaatgc attccttgct agctcagcgg gcggcttgtc cctaggaaga
15001 ggcgattcaa cacaccattg gaattttgca gacagaaaga gatttttgtt ttctgtttta
15061 tgactttttg aaaaagcttc tgggagttct gatttcctca gtcctttagg ttaaagcagc
15121 gccaggagga agctgacaga agcagcgttc ctgaagtggc cgaggttaaa cggaatcaca
15181 gaatggtcca gcacttttgc ttttttttct tttccttttc tttttttttt gtttgttttt
15241 tgttttgttt ttcccttgtg ggtgggtttc attgttttgg ttttctagtc tcactaagga
15301 gaaactttta ctggggcaaa gagccgatgg ctgccctgcc ccgggcaggg gccttcctat
15361 gaatgtaaga ctgaaatcac cagcgagggg gacagagagt gctggccacg gccttattaa
15421 aaaggggcag gccctctaac ttcaaaatgt ttttaaataa agtagacacc actgaacaag
15481 gaatgtactg aaatgacttc cttagggata gagctaaggg ataataactt gcactaaata
15541 catttaaata cttgattcca tgagtcagtt tattgtagtt tttgatttct gtaaaataag
15601 agaaactttt gtatttatta ttgaataagt gaatgaagct atttttaaat aaagttagaa
15661 gaaagccaag ctgctgctgt tacctgcaga actaacaaac cctgttactt tgtacagata
15721 tgtaaatatt ttgagaaaaa atacagtata aaaatagtta ttgaccaaat gctaccaggc
15781 tctgcagcag ctcgggggct tataaaatgt tcatagggat gttacaatat aattttgtgt
15841 tataaaatat gccattataa ttatgtaata accaaaattt caacctagag tgttgggggt
15901 tttttggaaa ccgcagtcta ttagtactca atggttttat acaccttact tctgacagag
15961 cggggcgtat gctacgacta caacttttat agctgttttg gtaatttaaa ctaatttttt
16021 catattatat tgttgcatcc ctacttcttc agtcaggttt ttttgtgctt acaatttgtg
16081 ataactgtga ataactgctt aaaaatacac ccaaatggag gctgaatttt ttcttcagca
16141 aaagtagttt tgattagaac tttgtttcag ccacagagaa tcatgtaaac gtaataggat
16201 catgtagcag aaacttaaat ctaacccttt agccttctat ttaacacaaa aatttgaaaa
16261 agttaaaaaa aaaaaggaga tgtgattatg cttacagctg caggactctg gcaatagggt
16321 ttttggaaga tgtaatttta aaatgtgttt gtatgaactg tttgtttaca tttctttaat
16381 aaaaaaaaca ctgttttgtg tttgcttgta gaaacttaat cagcattttg aaccaggtta
16441 gctttttatt ttgtacttaa aattctggta ctgacacttc acaggctaag tataaaatga
16501 agttttgtgt gcacaattca agtggactgt aaactgttgg tatattcagt gatgcagttc
16561 tgaacttgta tatggcatga tgtattttta tcttacagaa taaatcaatt gtatatattt
16621 ttctcttgat aaatagctgt atgaaatttg tttcctgaat atttttcttc tcttgtacaa
16681 tatcctgaca tcctaccagt atttgtccta ccgggttttt gttgttttct gttctgtata
16741 atagtatcta atgttggcaa aaattgaatt ttttgaagta tacagagtgt tatgggtttt
16801 ggaatttgtg gacacagatt tagaagatca ccatttacaa ataaaatatt ttacatctat
16861 aaaaaaaaaa aa
KAT8 (accession No. NM_032188):
(SEQ ID NO: 137)
        1 gtcacttccc ttcccgcgat ggcggcacag ggagctgctg cggcggttgc ggcggggact
      61 tcaggggtcg cgggggaggg cgagcccggg cccggggaga atgcggccgc tgaggggacc
    121 gccccatccc cgggccgcgt ctctccgccg accccggcgc gcggcgagcc ggaagtcacg
    181 gtggagatcg gagaaacgta cctgtgccgg cgaccggata gcacctggca ttctgctgaa
    241 gtgatccagt ctcgagtgaa cgaccaggag ggccgagagg aattctatgt acactacgtg
    301 ggctttaacc ggcggctgga cgagtgggta gacaagaacc ggctggcgct gaccaagaca
    361 gtgaaggatg ctgtacagaa gaactcagag aagtacctga gcgagctcgc agagcagcct
    421 gagcgcaaga tcactcgcaa ccaaaagcgc aagcatgatg agatcaacca tgtgcagaag
    481 acttatgcag agatggaccc caccacagca gccttggaga aggagcatga ggcgatcacc
    541 aaggtgaagt atgtggacaa gatccacatc gggaactacg aaattgatgc ctggtatttc
    601 tcaccattcc ccgaagacta tgggaaacag cccaagctct ggctctgcga gtactgcctc
    661 aagtacatga aatatgagaa gagctaccgc ttccacttgg gtcagtgcca gtggcggcag
    721 ccccccggga aagagatcta ccgcaagagc aacatctccg tgtacgaagt tgatggcaaa
    781 gaccataaga tttactgtca gaacctgtgt ctgctggcca agcttttcct ggaccataag
    841 acactgtact ttgacgtgga gccgttcgtc ttttacatcc tgactgaggt ggaccggcag
    901 ggggcccaca ttgttggcta cttctccaag gagaaggagt ccccggatgg aaacaatgtg
    961 gcctgcatcc tgaccttgcc cccctaccaa cgccgcggct acgggaagtt cctcatcgct
  1021 ttcagttatg agctctccaa gctggagagc acagtcggct ccccggagaa gccactgtct
  1081 gacctgggca agctcagcta ccgcagctac tggtcctggg tgctgctaga gatcctgcgg
  1141 gacttccggg gcacactgtc catcaaggac ctcagccaga tgaccagtat cacccaaaat
  1201 gacatcatca gtaccctgca atccctcaat atggtcaagt actggaaggg ccagcacgtg
  1261 atctgtgtca cacccaagct ggtggaggag cacctcaaaa gtgcccagta taagaaacca
  1321 cccatcacag tggactccgt ctgcctcaag tgggcacccc ccaagcacaa gcaagtcaag
  1381 ctctccaaga agtgagcagc ctggcccctg ctgtcggacc tgagcctcct ggctcccagc
  1441 ctgtaaatat gtatagacct gttttgtcat ttttttaata aagtcagttc tggtggccct
  1501 ggactttgga ggggaagggg aggccaagaa aaaaaaaaaa aaaaaa
KDM6A (accession No. NM_001291415):
(SEQ ID NO: 138)
        1 gtgtgacaca attacaacaa ctttgtgctg gtgccgggga agtttgtgtc tccaacgaat
      61 cccctcagtg ctccccagcc ccgcgcgctc cggccgttcc cgccgtcccc gcctgtggct
    121 gccccctgcc caaccccgcg atgtgaccct acagccgaaa gccgccgctg ccgacccggg
    181 ggctccgcag cccctgccgc cgccgccgcc gccttcaccg ccgccgcgtt gggatttttc
    241 gtcgccgccg cccgcggcgg aggaggaggc ggcgataaag ttggtgtgct ggtcccgcgc
    301 gcagattggg ggcgtcactg cgggccccgg tccgaggggg ggtgtcggcg ttggagttgt
    361 gaattcgctg cgtttccatg aaatcctgcg gagtgtcgct cgctaccgcc gccgctgccg
    421 ccgccgcttt cggtgatgag gaaaagaaaa tggcggcggg aaaagcgagc ggcgagagcg
    481 aggaggcgtc ccccagcctg acagccgagg agagggaggc gctcggcgga ctggacagcc
    541 gcctctttgg gttcgtgaga tttcatgaag atggcgccag gacgaaggcc ctactgggca
    601 aggctgttcg ctgctatgaa tctctaatct taaaagctga aggaaaagtg gagtctgatt
    661 tcttttgtca attaggtcac ttcaacctct tattggaaga ttatccaaaa gcattatctg
    721 cataccagag gtactacagt ttacagtctg actactggaa gaatgctgcc tttttatatg
    781 gtcttggttt ggtctacttc cattataatg catttcagtg ggcaattaaa gcatttcagg
    841 aggtgcttta tgttgatccc agcttttgtc gagccaagga aattcattta cgacttgggc
    901 ttatgttcaa agtgaacaca gactatgagt ctagtttaaa gcattttcag ttagctttgg
    961 ttgactgtaa tccctgcact ttgtccaatg ctgaaattca atttcacatt gcccacttat
  1021 atgaaaccca gaggaaatat cattctgcaa aagaagctta tgaacaactt ttgcagacag
  1081 agaatctttc tgcacaagta aaagcaactg tcttacaaca gttaggttgg atgcatcaca
  1141 ctgtagatct cctgggagat aaagccacca aggaaagcta tgctattcag tatctccaaa
  1201 agtccttgga agcagatcct aattctggcc agtcctggta tttcctcgga aggtgctatt
  1261 caagtattgg gaaagttcag gatgccttta tatcttacag gcagtctatt gataaatcag
  1321 aagcaagtgc agatacatgg tgttcaatag gtgtgctata tcagcagcaa aatcagccca
  1381 tggatgcttt acaggcctat atttgtgctg tacaattgga ccatggccat gctgcagcct
  1441 ggatggacct aggcactctc tatgaatcct gcaaccagcc tcaggatgcc attaaatgct
  1501 acttaaatgc aactagaagc aaaagttgta gtaatacctc tgcacttgca gcacgaatta
  1561 agtatttaca ggctcagttg tgtaaccttc cacaaggtag tctacagaat aaaactaaat
  1621 tacttcctag tattgaggag gcgtggagcc taccaattcc cgcagagctt acctccaggc
  1681 agggtgccat gaacacagca cagcaggcat gtaaacctca tcatccaaat actgaacctg
  1741 tattaggcct cagtcaaaca ccaatttcac agcaatcctt gccactacac atgattcctt
  1801 ctagccaagt agatgacctg tccagtcctg ccaagaggaa aagaacatct agtccaacaa
  1861 agaatacttc tgacaattgg agtggtggac atgctgtgtc acatcctcca gtacagcaac
  1921 aagctcattc atggtgtttg acaccacaga aattacagca tttggaacag ctccgcgcaa
  1981 atagaaataa tttaaatcca gcacagaaac tgatgctgga acagctggaa agtcagtttg
  2041 tcttaatgca acaacaccaa atgagaccaa caggagttgc acaggtacga tctactggaa
  2101 ttcctaatgg gccaacagct gactcatcac tgcctacaaa ctcagtctct ggccagcagc
  2161 cacagcttgc tctgaccaga gtgcctagcg tctctcagcc tggagtccgt cctgcctgcc
  2221 ctgggcagcc tttggccaat ggaccctttt ctgcaggcca tgttccctgt agcacatcaa
  2281 gaacgctggg aagtacagac actattttga taggcaataa tcatataaca ggaagtggaa
  2341 gtaatggaaa cgtgccttac ctgcagcgaa acgcactcac tctacctcat aaccgcacaa
  2401 acctgaccag cagcgcagag gagccgtgga aaaaccaact atctaactcc actcaggggc
  2461 ttcacaaagg tcagagttca cattcggcag gtcctaatgg tgaacgacct ctctcttcca
  2521 ctgggccttc ccagcatctc caggcagctg gctctggtat tcagaatcag aacggacatc
  2581 ccaccctgcc tagcaattca gtaacacagg gggctgctct caatcacctc tcctctcaca
  2641 ctgctacctc aggtggacaa caaggcatta ccttaaccaa agagagcaag ccttcaggaa
  2701 acatattgac ggtgcctgaa acaagcaggc acactggaga gacacctaac agcactgcca
  2761 gtgtcgaggg acttcctaat catgtccatc agatgacggc agatgctgtt tgcagtccta
  2821 gccatggaga ttctaagtca ccaggtttac taagttcaga caatcctcag ctctctgcct
  2881 tgttgatggg aaaagccaat aacaatgtgg gtactggaac ctgtgacaaa gtcaataaca
  2941 tccacccagc tgttcataca aagactgata actctgttgc ctcttcacca tcttcagcca
  3001 tttcaacagc aacaccttct ccaaaatcca ctgagcagac aaccacaaac agtgttacca
  3061 gccttaacag ccctcacagt gggctacaca caattaatgg agaagggatg gaagaatctc
  3121 agagccccat gaaaacagat ctgcttctgg ttaaccacaa acctagtcca cagatcatac
  3181 catcaatgtc tgtgtccata taccccagct cagcagaagt tctgaaggca tgcaggaatc
  3241 taggtaaaaa tggcttatct aacagtagca ttttgttgga taaatgtcca cctccaagac
  3301 caccatcttc accataccct cccttgccaa aggacaagtt gaatccacct acacctagta
  3361 tttacttgga aaataaacgt gatgctttct ttcctccatt acatcaattt tgtacaaatc
  3421 cgaacaaccc tgttacagta atacgtggcc ttgctggagc tcttaagtta gacctgggac
  3481 ttttctctac taaaactttg gtggaagcta acaatgaaca tatggtagaa gtgaggacac
  3541 agttgttgca gccagcagat gaaaactggg atcccactgg aacaaagaaa atctggcatt
  3601 gtgaaagtaa tagatctcat actacaattg ctaaatatgc acagtaccag gcctcctcat
  3661 tccaggaatc attgagagaa gaaaatgaaa aaagaagtca tcataaagac cactcagata
  3721 gtgaatctac atcgtcagat aattctggga ggaggaggaa aggacccttt aaaaccataa
  3781 agtttgggac caatattgac ctatctgatg acaaaaagtg gaagttgcag ctacatgagc
  3841 tgactaaact tcctgctttt gtgcgtgtcg tatcagcagg aaatcttcta agccatgttg
  3901 gtcataccat attgggcatg aacacagttc aactatacat gaaagttcca gggagcagaa
  3961 caccaggtca tcaggaaaat aacaacttct gttcagttaa cataaatatt ggcccaggtg
  4021 actgtgaatg gtttgttgtt cctgaaggtt actggggtgt tctgaatgac ttctgtgaaa
  4081 aaaataattt gaatttccta atgggttctt ggtggcccaa tcttgaagat ctttatgaag
  4141 caaatgttcc agtgtatagg tttattcagc gacctggaga tttggtctgg ataaatgcag
  4201 gcactgttca ttgggttcag gctattggct ggtgcaacaa cattgcttgg aatgttggtc
  4261 cacttacagc ctgccagtat aaattggcag tggaacggta cgaatggaac aaattgcaaa
  4321 gtgtgaagtc aatagtaccc atggttcatc tttcctggaa tatggcacga aatatcaagg
  4381 tctcagatcc aaagcttttt gaaatgatta agtattgtct tctaagaact ctgaagcaat
  4441 gtcagacatt gagggaagct ctcattgctg caggaaaaga gattatatgg catgggcgga
  4501 caaaagaaga accagctcat tactgtagca tttgtgaagt ggaggttttt gatctgcttt
  4561 ttgtcactaa tgagagtaat tcacgaaaga cctacatagt acattgccaa gattgtgcac
  4621 gaaaaacaag cggaaacttg gaaaactttg tggtgctaga acagtacaaa atggaggacc
  4681 tgatgcaagt ctatgaccaa tttacattag ctcctccatt accatccgcc tcatcttgat
  4741 attgttccat ggacattaaa tgagaccttt tctgctattc aggaaataac ccagttctgc
  4801 accactggtt tttgtagcta tctcgtaagg ctgctggctg aaaactgtgt ctatgcaacc
  4861 ttccaagtgc ggagtgtcaa ccaactggac gggagagagt actgctccta ctccaggact
  4921 ctcacaaagc tgatgagctg tacttcagaa aaaaataata atttccatgt tttgtatata
  4981 tctgacaaaa ctggcaacat cttacagact actgacttga agacaacctc ttttatattt
  5041 ctctatttct gggctgatga atttgttttc atctgtcttt tcccccttca gaattttcct
  5101 tggaaaaaaa atactagcct agctggtcat ttctttgtaa ggtagttagc aattttaagt
  5161 ctttctttgg tcaacttttt tttaatgtga aaagttaggt aagacacttt tttactgctt
  5221 ttatgttttt ctgtcttgtt ttgagaccat gatggttaca cttttggttc ctaaataaaa
  5281 tttaaaaaat taacagccaa gtcacaaagg taatggattg cacatagact aaggaataaa
  5341 cttcagattt gtgatttttg tttctaatct tgatgtaaat ttacactatt tataaataca
  5401 Tatttattgc Ttgaaaatat Ttgtgaatgg Aatgctgtta Ttttttccag Atttacctgc
  5461 cattgaaatt ttaaggagtt ctgtaatttc aaacactact cctattacat tttctatgtg
  5521 taaataaaac tgcttagcat tgtacagaaa cttttattaa aattgtttaa tgtttaaaga
  5581 gttttctatt gtttgagttt taaaaaagac tttatgtaca gtgcccagtt tttgttcatt
  5641 tttgaaatct gattatatat attttatata tacttatgta tgtatatata atatatatag
  5701 aaatctggat atatatgtat aaatctttag aacttaaatt tttctcgttt taagtttcac
  5761 atctatggta gatttttgag gtgtctactg taaagtattg cttacaaaaa gtatgattat
  5821 ttttaaagaa atatatatgg tatgtatcct caagacctaa aatgtcagac tggtttattg
  5881 ttaagttgca attactgcaa tgacagacca ataaacaatt gctgccaaaa tgtagtataa
  5941 a
NCOA6 (accession No. NM_014071):
(SEQ ID NO: 139)
        1 gtgaggccct gccgggtcgg gctgcgggcg gccgggcgcg ggcggcggga cagacgggcg
      61 cacgcgagga ctgacggacg gacgcaccga gggcggcggg cacgcacggc ccgggccggc
    121 gctccaaggc ccgcccggga gggccggggc cgcgctcaga attttgattt ggctgctggg
    181 ctgctacctt gaaatccaag ccctaaaaat gccagcttct ttggacttag aagatgacct
    241 ggataaatga taaaaattaa gaaagagatt ttgaagtttt cttattgtcc tcttggcata
    301 tgcttctgga ataatattca ccatggtttt ggatgacctt ccaaacttag aagacatcta
    361 tacttccttg tgttcatcaa caatggaaga ctcagagatg gattttgact ctggactaga
    421 agatgatgac acaaaaagtg atagtatttt ggaggattcc acaatttttg tggccttcaa
    481 aggaaatata gatgataaag acttcaaatg gaaattagat gcaatattga aaaacgtgcc
    541 caatttctta cacatggagt ccagcaagct aaaagtacag aaggtggagc cctggaacag
    601 cgtgcgtgtg acattcaaca tcccccggga agcagcggag cggctacgga tccttgctca
    661 gagcaacaac cagcagcttc gggatttagg gattctctcc gttcagattg aaggggaagg
    721 tgctattaac ctggctttgg ctcagaaccg aagccaagat gtgagaatga atggacccat
    781 gggagctgga aattcagtta ggatggaggc gggatttcct atggcaagtg gtccaggaat
    841 aataaggatg aacaaccctg ccactgttat gatacccccg ggtggaaatg tgtcatcttc
    901 catgatggca ccaggcccca atccagagct gcagcccagg actcctcgcc ctgcttctca
    961 gtcagatgca atggatccac tcctctctgg gctccatata cagcagcaaa gtcatccctc
  1021 aggatcttta gctcccccac atcacccaat gcagcctgtc tctgtgaaca gacaaatgaa
  1081 cccagctaat tttccccagc tgcagcagca gcagcaacaa caacaacagc agcagcagca
  1141 gcagcagcag caacaacagc aacagcagca acaacagttg caggcaagac ccccacagca
  1201 acatcagcag caacagccac agggaattcg accccagttt actgccccaa ctcaggtgcc
  1261 tgttcctcca ggctggaacc agctgccttc tggagccctt caacctcctc cagcccaggg
  1321 ttctctgggc acaatgactg caaaccaagg gtggaagaag gctcccttgc ccggcccaat
  1381 gcaacagcaa ctccaggcaa gaccatcctt agccacggta cagacgcctt cccaccctcc
  1441 ccctccatat ccctttggca gccagcaagc ctcacaagcc cacacaaact ttcctcagat
  1501 gagcaaccca ggccagttca cagctcctca gatgaagagt ttgcagggag ggccctctag
  1561 ggtcccaact cccttgcagc agccccacct caccaacaag tctcctgcct cctcaccctc
  1621 ctccttccag cagggatccc ctgcatcctc cccaacggtt aaccaaactc agcagcagat
  1681 gggaccaagg ccacctcaaa ataacccact tccccaggga tttcagcagc ctgtcagctc
  1741 tccgggtcgg aatcctatgg ttcaacaggg aaatgtgcca cctaacttca tggtgatgca
  1801 gcagcaacca ccaaaccagg ggccacagag tttacatcca ggcctaggag gaatgcctaa
  1861 acgcctccca cctggcttct cagcaggaca ggccaatccg aactttatgc aaggtcaggt
  1921 gccttcgacc acagcaacca cccctgggaa ttcaggagcc cctcagctgc aagcaaatca
  1981 aaatgtccag catgcaggtg gtcaaggagc tggtcctcct caaaaccaga tgcaggtgtc
  2041 ccacgggccg ccaaatatga tgcagcccag cctcatggga attcatggca acatgaacaa
  2101 tcagcaggct ggtacttctg gggttcctca agtgaacctc agcaacatgc aaggccagcc
  2161 ccagcagggc ccaccatctc agctgatggg catgcaccag caaatcgtgc cctcccaggg
  2221 ccagatggtc cagcaacaag gaaccttgaa ccctcagaac cctatgatcc tttcaagggc
  2281 ccagcttatg ccacagggcc agatgatggt gaaccccccg agccaaaatc ttgggccctc
  2341 gccccaaagg atgaccccac ccaagcagat gctttcccag cagggcccac aaatgatggc
  2401 gccacataac cagatgatgg ggcctcaggg gcaggttttg ctccaacaga acccaatgat
  2461 agagcagatt atgaccaatc aaatgcaggg gaataagcag cagtttaaca ctcagaacca
  2521 gtccaatgtc atgccgggac cagcccagat aatgagggga ccaactccaa acatgcaagg
  2581 aaatatggtg cagtttacgg gacagatgtc aggacagatg ctgccccagc aagggcctgt
  2641 gaacaacagt ccatctcagg ttatgggcat tcagggacag gtcctgcggc caccagggcc
  2701 cagcccacac atggcccagc agcatggtga tcctgctact acagcaaata acgatgtcag
  2761 tttatctcag atgatgcctg atgttagcat tcaacaaacc aacatggtcc cccctcatgt
  2821 gcaggccatg cagggaaaca gtgcctcggg aaaccacttc tcaggccatg ggatgtcttt
  2881 caatgcacct ttcagtggag ctcccaatgg aaatcagatg tcctgtggtc aaaatccagg
  2941 cttcccagtc aataaggatg tcacgctaac gagcccattg ttggtcaact tattgcagag
  3001 tgacatatct gcaggccatt ttggggtaaa caataagcaa aataatacca acgcaaataa
  3061 accgaagaag aagaaacccc ctcggaagaa gaaaaatagt cagcaagatc taaacacccc
  3121 agatactcgc ccagctggtc tggaagaggc tgatcagcca ccgttgcctg gagaacaagg
  3181 aattaacttg gataactcag gccctaaact gccagaattt tcaaaccggc caccaggtta
  3241 tccttctcaa ccagttgaac agaggccact tcagcagatg cctcctcaac tcatgcagca
  3301 tgtggcaccc ccaccacagc caccacagca gcagccacag ccacaactgc ctcagcagca
  3361 gcagccacca cctcccagtc agccacagtc tcagcagcag cagcagcagc agcaacaaat
  3421 gatgatgatg ctcatgatgc agcaggatcc caaatcagtt aggcttccag tctctcaaaa
  3481 tgtccatcct ccaaggggcc ccctgaaccc cgactcccag agaatgccca tgcaacagag
  3541 tggcagtgtg cctgtcatgg tcagtctgca aggacctgcc tccgtgccac catcacctga
  3601 taaacaaaga atgccaatgc ctgtgaatac tcccttggga agcaattcaa ggaaaatggt
  3661 ctatcaggag agcccgcaga atccttccag ctcgccactg gcggagatgg cctcactccc
  3721 tgaagcaagt ggcagtgaag caccatctgt cccaggaggc ccaaacaaca tgccttcaca
  3781 tgtagtactt ccccagaatc agttaatgat gacagggcca aaacctggac catcgcccct
  3841 ttcagcaact caaggtgcaa ctccccagca accccctgta aattccctgc ccagctctca
  3901 cggccaccac ttcccaaatg tggctgcgcc aacccagaca tctaggccca aaacaccaaa
  3961 cagagccagc cccagaccct attatcctca gacacccaac aaccgccctc ccagcacaga
  4021 accttcagaa atcagtctgt caccagaaag actcaatgcc tccatagcag gactcttccc
  4081 tccacagatt aatattcctt tacctcctag gccaaattta aacaggggct ttgatcaaca
  4141 aggcctaaat ccaacaactt tgaaggccat cgggcaagca ccttcaaatc ttaccatgaa
  4201 tccttccaat tttgctaccc cacaaactca caaattagat tctgtggtag tgaattctgg
  4261 aaagcagtct aattctggag caacaaaacg ggcaagtcca agcaacagtc gcaggtctag
  4321 tcctgggtcc agtaggaaaa ccactccaag ccctgggagg caaaattcaa aagcccctaa
  4381 acttactctg gcctctcaga caaatgcagc cctattgcag aatgtggagt tgccgagaaa
  4441 tgtattggtc agtcccactc ctctggccaa tccccctgta cctgggagct ttcctaacaa
  4501 cagtgggctg aatcctcaga attctactgt gtctgtggct gcagttgggg gtgttgttga
  4561 ggataacaag gagagcttga atgtgcctca ggacagtgat tgccagaatt cccagagtag
  4621 gaaggaacag gtaaacattg aactaaaagc agtccctgcc caagaagtta aaatggttgt
  4681 ccctgaagat cagtccaaaa aggatgggca gccttcggat cctaacaaac ttcccagtgt
  4741 cgaagagaac aaaaatttgg tgtctcctgc tatgagggaa gcaccaacat cgttaagtca
  4801 acttcttgac aactctggag ctcccaatgt gacaattaaa ccccctgggc ttacagatct
  4861 ggaagtaaca cctccagtag tttctgggga ggacctcaaa aaagcatctg tcattcccac
  4921 actgcaggat ctgtcttctt ctaaagaacc ttctaattcc ctaaacttac ctcacagtaa
  4981 tgagctgtgt tcatcccttg tgcatcccga attgagtgag gtcagttcta acgttgcacc
  5041 aagcatccct ccagtaatgt caagacctgt tagctcttcc tccatttcca ctcccttgcc
  5101 cccaaatcaa ataactgtat ttgtcacttc caatcccatc acaacttcag ctaacacatc
  5161 agcagctttg ccaactcact tgcagtctgc attgatgtca acagttgtca caatgcccaa
  5221 tgcgggtagc aaggttatgg tttctgaggg acagtcagct gctcagtcta atgcccggcc
  5281 tcagttcatt acacctgtct ttatcaattc atcctcaata attcaggtta tgaaaggatc
  5341 acagccaagc acaattcctg cagccccact gacaaccaac tctggcctga tgcctccctc
  5401 tgttgcagtt gttggccctt tacacatacc tcagaacata aaattttctt ctgctcctgt
  5461 accgcctaat gccctctcca gtagtcctgc tccaaacatc cagacaggtc gacctttggt
  5521 ccttagctca cgagccaccc ctgttcagct tccttcccct ccttgtacgt cttctccagt
  5581 tgtcccttct catccccctg tgcagcaagt gaaagaattg aatccagatg aggctagccc
  5641 tcaggtgaac acctcagcag atcagaacac tcttccctct tcacagtcaa ccacaatggt
  5701 ttctcccctt ttgaccaata gtccagggtc ctctggcaac cggcgaagcc cagtctcgtc
  5761 tagtaagggc aaaggaaaag tggacaaaat tggccaaatt ttttgacca aggcatgtaa
  5821 gaaagttaca ggctctcttg agaaagggga agaacaatat ggtgcagatg gagagactga
  5881 aggccaaggg ctagacacca cagctccggg gctcatggga acagagcagt tatccacaga
  5941 gctggacagt aaaaccccaa cgcccccagc acccactctg ctaaaaatga cctctagccc
  6001 tgtgggcccg ggcactgcct cagcaggacc cagcttacct ggcggtgctc tccccaccag
  6061 tgtacgctcg atagtaacca ctctggtacc ctccgagctc atctccgccg taccgaccac
  6121 aaaaagcaat catggtggca tagcatctga gtcacttgcg ggtggcctag tggaggagaa
  6181 ggtgggatcc catccagaac ttctacccag catagccccg tcgcagaatt tagtctcaaa
  6241 ggaaacttca accacagcac tgcaggcctc tgttgccaga ccagagctgg aggtaaatgc
  6301 tgccatagtc tctggacaaa gcagtgagcc caaagagata gttgaaaagt ccaaaatccc
  6361 aggccgaaga aactcccgaa ctgaagagcc aactgtggcc tctgaaagtg tggaaaatgg
  6421 acatcgtaaa cgatcttctc gacctgcttc agcctccagc tctactaaag acataaccag
  6481 tgcggtgcaa tccaagcgaa gaaaatccaa gtaaacaagc aggactgcga cttgatactt
  6541 ggaaatgtgt gtgactttta caaagagcaa ttttgagctg tgactttttt aaatcaattt
  6601 ctgtacagtt agtaatttta ataatgtggc ccttttccta gtccctgcaa cctgtttcat
  6661 aaagtgcaat ggggaaagca ggactgttga gcccttttgg tgttgcgagt tgaagttcaa
  6721 ggtttctaaa atgttgtctt gtattgaaag gagctaatgc cattataaat gttactagtt
  6781 ttcacatttc ctaagcagcc tagagtacag ggtgagcatt tttagatctc ctaatgatgt
  6841 attgtgccgt ggaagtactg tgtgtgaata gcagtagtgg gggcaaaagc aatcttctca
  6901 tttggaaatg ttgtaaataa ttttattata tagtgttttg gatgtatttg ttgtagaaat
  6961 ggaccagtga ataaagagaa tctaaggatt tgtacaatgt gaaataacgt gttaaataaa
  7021 tgtcattgtc atagaacata aagttatgtt attggtaagg gaaaaaaaaa a
PAGR1 (accession No. NM_024516):
(SEQ ID NO: 140)
        1 ggcgccgtgt ccgggtgtgg agaggggcgt cgtggaagcg agaagagtgg cccgtccctc
      61 tcctccccct ttccctcttt cggaaagtgg tttctgcggg gcccgggagc ctcggagtac
    121 cgaacctcga tctccggggc ggggtccttg gtggggactg agcgccccct cccggggacg
    181 ggcggtctgg ccgcggagtc ccctgcggga gcgtgattgg ctggaaacgg tcccgaaccc
    241 ccaggggagc ccgatccctg ggggaccctg gcttcggact ccagtatctg tcgtcgcagg
    301 gtccctgccc tagtggccta tgtcccttgc tcggggccat ggagacactg cggccagtac
    361 ggcggcgcct ctgtctgaag aaggggaagt gacctccggc ctccaggctc tggccgtgga
    421 ggataccgga ggcccctctg cctcggccgg taaggccgag gacgaggggg aaggaggccg
    481 agaggagacc gagcgtgagg ggtccggggg cgaggaggcg cagggagaag tccccagcgc
    541 tgggggagaa gagcctgccg aggaggactc cgaggactgg tgcgtgccct gcagcgacga
    601 ggaggtggag ctgcctgcgg atgggcagcc ctggatgccc ccgccctccg aaatccagcg
    661 gctctatgaa ctgctggctg cccacggtac tctggagctg caagccgaga tcctgccccg
    721 ccggcctccc acgccggagg cccagagcga agaggagaga tccgatgagg agccggaggc
    781 caaagaagag gaagaggaaa aaccacacat gcccacggaa tttgattttg atgatgagcc
    841 agtgacacca aaggactccc tgattgaccg gagacgcacc ccaggaagct cagcccggag
    901 ccagaaacgg gaggcccgcc tggacaaggt gctgtcggac atgaagagac acaagaagct
    961 ggaggagcag atccttcgta ccgggaggga cctcttcagc ctggactcgg aggaccccag
  1021 ccccgccagc cccccactcc gatcctccgg gagtagtctc ttccctcggc agcggaaata
  1081 ctgattccca ctgctcctgc ctctagggtg cagtgtccgt acctgctgga gcctgggccc
  1141 tccttcccca gcccagacat tgagaaactt gggaagaaga gagaaacctc aagctcccaa
  1201 acagcacgtt gcgggaaaga ggaagagaga gtgtgagtgt gtgtgtgtgt tttttctatt
  1261 gaacacctgt agagtgtgtg tgtgtgtttt ctattgaaca cctatagaga gagtgtgtgt
  1321 gttttctatt gaacatctat atagagagag tgtgtgagtg tgtgttttct attgaacacc
  1381 tattcagaga cctggactga attttctgag tctgaaataa aagatgcaga gctatcatct
  1441 cttaaaagga ggggctgtag ctgtagctca acagttaggc cccacttgaa gggagaggca
  1501 gaattgtact cacccagatt ggaaaatgaa agccagatgg gtagaggtgc cctcagttag
  1561 cacctgtccc atctcgggcc ctccaactcc tcccagtccc actccagtgc agccagctgg
  1621 ctccaaggta gaaacccatg agcactcagg gagcagtgtg ccttcagctg cagcagaagc
  1681 agcccggagg ataaaatgag aaccagctgc acacgggccc tttaactccc aagccccacc
  1741 cctgggcttg gcctgccttg ccctgccggg aagtgatccc caaggcaggg tgagagttcc
  1801 ccatctgagg cgtttgttgc agctacctgc acttctagat gtgagtacat tgtactagcc
  1861 ccccaaaccc caaatcaggg gcagatcttt gtatcccttg aggctctctt tagtcctgtc
  1921 ttgctttgaa gggccttgct tctgctgggg cagggaaaac atgtctgaat cagagtgggg
  1981 aaggaggatg ggtggtggct ttgcttttgg aggtttcact ttccaatagt tgggagtctt
  2041 ctgggttttg aagtaaaggc agattaacac caacaccggt cccccacccc cctgcaactc
  2101 tcaggcctct ctctgacttc agggtcccac ctgggaaatc aggtggggaa ccttacaggg
  2161 tcattcagac cccatcttag ccctagatcg gtgcttgctc tactcacctg cactgtcctg
  2221 gggacctggg ctctggcctg tcaccttgag ctccaagaat gtgacctgta cccattcagg
  2281 ccccttaact ctgacagatg agggtttctt actcctccat gcagggctgg gccagctgtt
  2341 ggtctcagtc gatcattcag gaagtcatta gcagagtgat ttccagaagg cgtagaattt
  2401 agtgaccaag gttctttcct ttttgggagg agaaagtgaa aactaggatg ctcagctgga
  2461 cccaccagcc tgagattctg gggattttag agctgtccct tggggagcca agcacttggg
  2521 ggtggaggtg atagcgaggc tgatggcccc tgtgttctca gctctctgcc tgggtagccc
  2581 ctgggtgatg ggggagaggc cagctgtcac gtggggtatc aggtggctct gccagaaact
  2641 cccttggcac acagagcact gggtcggccc tcgggtgtgg ctgtttgggc aggacagccc
  2701 tctgtatgta gccttgagca ggtagggggg ccaccttgag tgggtggccc agagacagcc
  2761 tcagggctcc aaggtaacgg ggtgctcagg ttatcttggg tgctgccctc ccaggttctg
  2821 ggggagcaga ggctgggcgc tggcccaact tacaggaaac actcaccttt gaactgccat
  2881 tagcaccatc tgggcagtac acagccccac ccaggtcctc tagttcttgt tctcggctta
  2941 gaatctttgt gtttctgcct gagaagccac tgcctcctag tttgtggtct ctacagttat
  3001 agccaggttg gacttccggc tccgtccttt gataactgtg tgctcttggg caaatttctt
  3061 aacttgcagg ttcttgtgag gataacatga gttaattgag ggcacttaac actacctggc
  3121 acagattaag ctcatctgaa gtgggagctg ttacttaggg gcgtttgcct agaacacagg
  3181 gtccagaggc tctctcccgg aaacttagac ccagtgagtc agaagtgagg cctgcaaaaa
  3241 gcagcaggag tggggttaag aattccagcc tagggctgga tgcggtggct caggcctgta
  3301 atcccagtac tttgggaggc ccgaatggga ggatggcttg aggccaggag ttccagacca
  3361 gcctgagcaa catagcgaga ccctgtctct gtttgtgtgt gtgtggttgg ggttttgttt
  3421 tttttttttt tttaaagaat tatagctcag tcctatgatt aggcaagttg agaaaatatt
  3481 gatgaagatc aggggtgctg aagcctggtt cctggggtcg cttctgatct aggcggttct
  3541 tgcctctggt gactggtgtt aattggcagg agtgggagga gggaggacaa gtggaagtct
  3601 aggctggctg agctgttctg tctcgaaaag ttcctaaaac tgtgctgctt taaaaaaaaa
  3661 aaaagtaatt tatgagacac attctcaatt tccattaatc atctcctaaa gggggtaaac
  3721 caggaagccg ctgggtgaaa acaggctgtt ggcaattcct gagtcatgtg acccattctc
  3781 taaagactag aatatttaac ttaaatcagt gagaaactct gtgaaaaaaa aaaaaaaaaa
  3841 aaa
PAXIP1 (accession No. NM_007349):
(SEQ ID NO: 141)
        1 cggggccggg cgccgccgcg gagcctcccg ggccgccgcg atcatgtcgg accaggcgcc
      61 caaagttcct gaggagatgt tcagggaggt caagtattac gcggtgggcg acatcgaccc
    121 gcaggttatt cagcttctca aggctggaaa agcgaaggaa gtttcctaca atgcactagc
    181 ctcacacata atctcagagg atggggacaa tccagaggtg ggagaagctc gggaagtctt
    241 tgacttacct gttgtaaagc cttcttgggt gattctgtcc gttcagtgtg gaactcttct
    301 gccagtaaat ggtttttctc cagaatcatg tcagattttt tttggaatca ctgcctgcct
    361 ttctcaggtg tcatctgaag acagaagtgc cctgtgggct ttggttacgt tctatggggg
    421 agattgccag ctaaccctca ataagaaatg cacgcatttg attgttccag agccaaaggg
    481 ggagaaatac gaatgtgctt taaagcgagc aagtattaaa attgtgactc ctgactgggt
    541 tctggattgc gtatcagaga aaaccaaaaa ggacgaagca ttttatcatc ctcgtctgat
    601 tatttatgaa gaggaagaag aggaagagga agaggaggag gaagtagaaa atgaggaaca
    661 agattctcag aatgagggta gtacagatga gaagtcaagc cctgccagct ctcaagaagg
    721 gtctccttca ggtgaccagc agttttcacc taaatccaac actgaaaaat ctaaagggga
    781 attaatgttt gatgattctt cagattcatc accggaaaaa caggagagaa atttaaactg
    841 gaccccggcc gaagtcccac agttagctgc agcaaaacgc aggctgcctc agggaaagga
    901 gcctgggttg attaacttgt gtgccaatgt cccacccgtc ccaggtaaca ttttgccccc
    961 tgaggtccgg ggtaatttaa tggctgctgg acaaaacctc caaagttctg aaagatcaga
  1021 aatgatagct acctggagtc cagctgtacg gacactgagg aatattacta ataatgctga
  1081 cattcagcag atgaaccggc catcaaatgt agcacatatc ttacagactc tttcagcacc
  1141 tacgaaaaat ttagaacagc aggtgaatca cagccagcag ggacatacaa atgccaatgc
  1201 agtgctgttt agccaagtga aagtgactcc agagacacac atgctacagc agcagcagca
  1261 ggcccagcag cagcagcagc agcacccggt tttacacctt cagccccagc agataatgca
  1321 gctccagcag cagcagcagc agcagatctc tcagcaacct tacccccagc agccgccgca
  1381 tccattttca cagcaacagc agcagcagca gcaagcccat ccgcatcagt tttcacagca
  1441 acagctacag tttccacagc aacagttgca tcctccacag cagctgcatc gccctcagca
  1501 gcagctccag ccctttcagc agcagcatgc cctgcagcag cagttccatc agctgcagca
  1561 gcaccagctc cagcagcagc agcttgccca gctccagcag cagcacagcc tgctccagca
  1621 gcagcagcaa cagcagattc agcagcagca gctccagcgc atgcaccagc agcagcagca
  1681 gcagcagatg caaagtcaga cagcgccaca cttgagtcag acgtcacagg cgctgcagca
  1741 tcaggttcca cctcagcagc ccccgcagca gcagcagcaa cagcagccac caccatcgcc
  1801 tcagcagcat cagctttttg gacatgatcc agcagtggag attccagaag aaggcttctt
  1861 attgggatgt gtgtttgcaa ttgcggatta tccagagcag atgtctgata agcaactgct
  1921 ggccacctgg aaaaggataa tccaggcaca tggcggcact gttgacccca ccttcacgag
  1981 tcgatgcacg caccttctct gtgagagtca agtcagcagc gcgtatgcac aggcaataag
  2041 agaaagaaag agatgtgtta ctgcacactg gttaaacaca gtcttaaaga agaagaaaat
  2101 ggtaccgccg caccgagccc ttcacttccc agtggccttc ccaccaggag gaaagccatg
  2161 ttcacagcat attatttctg tgactggatt tgttgatagt gacagagatg acctaaaatt
  2221 aatggcttat ttggcaggtg ccaaatatac gggttatcta tgccgcagca acacagtcct
  2281 catctgtaaa gaaccaactg gtttaaagta tgaaaaagcc aaagagtgga ggataccctg
  2341 tgtcaacgcc cagtggcttg gcgacattct tctgggaaac tttgaggcac tgaggcagat
  2401 tcagtatagt cgctacacgg cattcagtct gcaggatcca tttgccccta cccagcattt
  2461 agttttaaat cttttagatg cttggagagt tcccttaaaa gtgtctgcag agttgttgat
  2521 gagtataaga ctacctccca aactgaaaca gaatgaagta gctaatgtcc agccttcttc
  2581 caaaagagcc agaattgaag acgtaccacc tcccactaaa aagctaactc cagaattgac
  2641 cccttttgtg cttttcactg gattcgagcc tgtccaggtt caacagtata ttaagaagct
  2701 ctacattctt ggtggagagg ttgcggagtc tgcacagaag tgcacacacc tcattgccag
  2761 caaagtgact cgcaccgtga agttcctgac ggcgatttct gtcgtgaagc acatagtgac
  2821 gccagagtgg ctggaagaat gcttcaggtg tcagaagttc attgatgagc agaactacat
  2881 tctccgagat gctgaggcag aagtactttt ctctttcagc ttggaagaat ccttaaaacg
  2941 ggcacacgtt tctccactct ttaaggcaaa atatttttac atcacacctg gaatctgccc
  3001 aagtctttcc actatgaagg caatcgtaga gtgtgcagga ggaaaggtgt tatccaagca
  3061 gccatctttc cggaagctca tggagcacaa gcagaactcg agtttgtcgg aaataatttt
  3121 aatatcctgt gaaaatgacc ttcatttatg ccgagaatat tttgccagag gcatagatgt
  3181 tcacaatgca gagttcgttc tgactggagt gctcactcaa acgctggact atgaatcata
  3241 taagtttaac tgatggcgtc taggctgccg tgcatgtcga ctcctgcggt gcggggctgg
  3301 ctgtctggct ggcgaggagc tgctgcgctt ccttcacatg ctcttgtttt ccagctgctt
  3361 tcctggggga tcagactgtg aagcaggaag acagatataa taaatatact gcatcttttt
  3421 aagatgtgca attttattct gaggaaacat aaattatgtt ttgtattata tgactttaag
  3481 agcccacatt aggttttatg attcatttgc caggttttta aatgttttca caaaactgtt
  3541 acgggacttc aactagaaat aaaatggtgt aaataaagac cttgctatct ctaaattatg
  3601 gatgttaaag atttgaaatg ttttgtactt tgattatttt tatttcttat actctgtttt
  3661 cttttatatt gatatcttgc ccacatttta aataaatgta cttttgaact taaaaaaaaa
  3721 aaa
ASH1L (accession No. NM_018489):
(SEQ ID NO: 142)
        1 aggagtggaa ggttgagggg ggcgctaggc gcccttcgct ccctccctct ggaggagctg
      61 ccgccgccac cgccgccact ctgctgctgc cgccgccgcc gccgccgctc ccgccgccat
    121 tttgggttcg ctttgcggag gggagacgat cccagtctcg gttgcgggac ccgcctcccc
    181 tcagtttgcc ccctttagcc ttccaccttt cccttctcct ctctcgcatt tccgccagtc
    241 agcttacccg ctggccgcct cctgacaagc gggagggatc cgccgtggac ccagggaagc
    301 ggaggagcct ggcggccacc ccctcttccc cacttccctg cactctcatc gctctcggcc
    361 tcggcctcgg cctccgacac gagaaagatg ctggtttcga gttttggaga tccttgtttt
    421 ttatggaaca cagttctgta aaattttcat aagattcctt ggcaataaca tacgcttgtg
    481 atggacccta gaaatactgc tatgttagga ttgggttctg attccgaagg tttttcaaga
    541 aagagtcctt ctgccatcag tactggcaca ttggtcagta agagagaagt agagctagaa
    601 aaaaacacaa aggaggaaga ggaccttcgc aaacggaatc gagaaagaaa catcgaagct
    661 gggaaagatg atggtttgac tgatgcacag caacagtttt cagtgaaaga aacaaacttt
    721 tcagagggaa atttaaaatt gaaaattggc ctccaggcta agagaactaa aaaacctcca
    781 aagaacttgg agaactatgt atgtcgacct gccataaaaa caactattaa gcacccaagg
    841 aaagcactta aaagtggaaa gatgacggat gaaaagaatg aacactgtcc ttcaaaacga
    901 gacccttcaa agttgtacaa gaaagcagat gatgttgcag ccattgaatg ccagtctgaa
    961 gaagtcatcc gtcttcattc acagggagaa aacaatcctt tgtctaagaa gctgtctcca
  1021 gtacactcag aaatggcaga ttatattaat gcaacgccat ctactcttct tggtagccgg
  1081 gatcctgatt taaaggacag agcattactt aatggaggaa ctagtgtaac agaaaagttg
  1141 gcacagctga ttgctacctg tcctccttcc aagtcttcca agacaaaacc gaagaagtta
  1201 ggaactggca ctacagcagg attggttagc aaggatttga tcaggaaagc aggtgttggc
  1261 tctgtagctg gaataataca taaggactta ataaaaaagc caaccatcag cacagcagtt
  1321 ggattggtaa ctaaagatcc tgggaaaaag ccagtgttta atgcagcagt aggattggtc
  1381 aataaggact ctgtgaaaaa actgggaact ggcactacag cggtattcat taataaaaac
  1441 ttaggcaaaa agccaggaac tatcactaca gtaggactgc taagcaaaga ttcaggaaag
  1501 aagctaggaa ttggtattgt tccaggttta gtgcataaag agtctggcaa gaagttagga
  1561 cttggcactg tggttggact ggttaataaa gatttgggaa agaaattggg ttctactgtt
  1621 ggcctagtgg ccaaggactg tgcaaagaag attgtagcaa gttcagcaat gggattggtt
  1681 aataaggaca ttggaaagaa actaatgagt tgtcctttgg caggtctgat cagtaaagat
  1741 gccataaacc ttaaagccga agcactgctc cccactcagg aaccgcttaa ggcttcttgt
  1801 agtacaaaca tcaataatca ggaaagtcag gaactttctg aatccctgaa agatagtgcc
  1861 accagcaaaa cttttgaaaa gaatgttgta cggcagaata aagaaagcat attggaaaag
  1921 ttctcagtac gaaaagaaat cattaatttg gagaaagaaa tgtttaatga aggaacatgc
  1981 attcagcaag acagtttctc atccagtgaa aagggatctt atgaaacctc aaagcatgaa
  2041 aagcagcctc ctgtatattg cacttctccg gactttaaaa tgggaggtgc ttctgatgta
  2101 tctaccgcta aatccccatt cagtgcagta ggagaaagca atctcccttc cccatcacct
  2161 actgtatctg ttaatccttt aaccagaagt ccccctgaaa cttcttcaca gttggctcct
  2221 aatccattac ttttaagttc tactacagaa ctaatcgaag aaatttctga atctgttgga
  2281 aagaaccagt ttacttctga aagtacccac ttgaacgttg gtcataggtc agttggtcat
  2341 agtataagta ttgaatgtaa agggattgat aaagaggtaa atgattcaaa aactacccat
  2401 atagatattc caagaataag ctcttccctt ggaaaaaagc caagtttgac ttctgaatcc
  2461 agcattcata ctattactcc ttcagttgtt aacttcacta gtttatttag taataagcct
  2521 tttttaaaac tgggtgcagt atctgcatca gacaaacact gccaagttgc tgaaagccta
  2581 agtactagtt tgcagtccaa accattaaaa aaaagaaaag gaagaaaacc tcggtggact
  2641 aaagtggtgg caagaagcac atgccggtct ccaaaagggc tagaattaga aagatcagag
  2701 ctttttaaaa acgtttcatg tagctcacta tcaaatagta attctgagcc agccaagttt
  2761 atgaaaaaca ttggaccccc ttcatttgta gatcatgact tccttaaacg ccgattgcca
  2821 aagttgagca aatccacagc tccatctctt gctctcttag ctgatagtga aaaaccatct
  2881 cataagtctt ttgctactca caaactatcc tccagtatgt gtgtctctag tgaccttttg
  2941 tctgatattt ataagcccaa aagaggaagg cctaaatcta aggagatgcc tcaactggaa
  3001 gggccaccta aaaggacttt aaaaatccct gcttctaaag tgttttcttt acagtctaag
  3061 gaagaacaag aacccccaat tttacagcca gaaattgaaa tcccttcctt caaacaaggt
  3121 ctgtctgtgt ctccttttcc aaaaaagaga ggcaggccta agaggcaaat gaggtcacca
  3181 gtcaagatga agccacctgt actgtcagtg gctccatttg ttgccactga aagtccaagc
  3241 aagctagaat ctgaaagtga caaccataga agtagcagtg atttctttga gagcgaggat
  3301 caacttcagg atccagatga cctagatgac agtcataggc caagtgtctg tagtatgagt
  3361 gaccttgaga tggaaccaga taaaaaaatt accaagagaa acaatggaca attaatgaaa
  3421 acaattatcc gcaaaataaa taaaatgaag actttaaaga gaaagaaact gttgaatcag
  3481 attctttcaa gttctgtaga atcaagtaat aaagggaaag tgcaatccaa actccataat
  3541 acggtatcaa gtcttgctgc cacatttggc tctaaattgg gccaacagat aaatgtcagc
  3601 aagaaaggaa ccatttatat aggaaagaga agaggtcgca aaccaaaaac tgtcttaaat
  3661 ggtattcttt ctggtagtcc tactagcctt gctgttcttg agcaaacagc tcaacaggca
  3721 gctgggtcag cattaggaca gattcttccc ccattactgc cttcatctgc tagtagttct
  3781 gagattcttc catcacctat ttgctctcag tcttctggga ctagtggagg tcagagccct
  3841 gtaagtagtg atgcaggttt tgttgaaccc agttcagtgc catatttgca tttacactcc
  3901 agacagggca gtatgattca gactcttgca atgaagaagg cctcaaaggg gaggaggcgg
  3961 ttatctcctc ctactttgtt gccaaattct ccttcgcact tgagtgaact cacatctcta
  4021 aaagaagcta ctccttcccc aatcagtgag tctcatagtg atgagaccat tcccagtgat
  4081 agtggaattg gaacagataa taacagcaca tcagacaggg cagagaaatt ttgtgggcaa
  4141 aaaaagagga ggcattcttt tgagcatgtt tctctgattc cccctgaaac ctctacagtg
  4201 ctaagcagtc ttaaagaaaa acataaacac aaatgtaagc gcaggaatca tgattacctc
  4261 agctatgaca agatgaaaag gcagaaacga aaacggaaaa agaaatatcc ccagcttcga
  4321 aatagacagg atccagactt tattgcagag ctggaggaac taataagtcg cctaagtgaa
  4381 attcggatca ctcatcgaag tcatcatttt atcccccgag atcttctgcc aactatcttt
  4441 cgaatcaact ttaatagttt ctatacacat ccttctttcc ccttagaccc tttgcactac
  4501 attcgaaaac ctgacttaaa aaagaaaaga gggagacccc ctaagatgag ggaggcaatg
  4561 gctgaaatgc cttttatgca cagccttagt tttcctcttt ctagtactgg attctatcca
  4621 tcttatggta tgccttactc tccttcaccc cttacagctg ctcccatagg attaggttac
  4681 tatggaaggt atcctcccac tctttatcca cctcctccat ctccttcttt caccacgcca
  4741 cttccacctc cttcctatat gcatgctggt catttacttc tcaatcctgc caaataccat
  4801 aagaaaaagc ataagctact tcgacaggag gcctttctta caaccagcag gactcccctc
  4861 ctttccatga gtacctaccc cagtgttcct cctgagatgg cctatggttg gatggttgag
  4921 cacaaacaca ggcaccgtca caaacacaga gaacaccgtt cttctgaaca accccaggtt
  4981 tctatggaca ctggctcttc ccgatctgtc ctggaatctt tgaagcgcta tagatttgga
  5041 aaggatgctg ttggagagcg atataagcat aaggaaaagc accgttgtca catgtcctgc
  5101 cctcatctct ctccttcaaa aagcttaata aacagagagg aacagtgggt ccaccgagag
  5161 ccttcagaat ctagtccatt ggccttggga ttgcagacac ctttacagat tgactgttca
  5221 gaaagttctc caagcttatc ccttggagga ttcactccca actctgagcc agccagcagt
  5281 gatgaacata caaacctttt cacaagtgca ataggcagct gcagagtttc aaaccctaac
  5341 tccagtggcc ggaagaaatt aactgacagc cctggactct tttctgcaca ggacacttca
  5401 ctaaatcggc ttcacagaaa ggagtcactg ccttctaacg aaagggcagt acagactttg
  5461 gcaggctccc agccaacctc tgataaaccc tcccagcggc catcagagag cacaaattgt
  5521 agccctaccc ggaaaaggtc ttcatctgag agtacttctt caacagtaaa cggagttccc
  5581 tctcgaagtc caagattagt tgcttctggg gatgactctg tggatagtct gctgcagcgg
  5641 atggtacaaa atgaggacca agagcccatg gagaaaagta ttgatgctgt gattgcaact
  5701 gcctctgcac caccttcttc cagtccaggc cgtagccaca gcaaggaccg aaccctggga
  5761 aaaccagaca gccttttagt gcctgcagtc acaagtgact cttgcaataa tagcatctca
  5821 ctcctatctg aaaagttgac aagcagctgt tccccccatc atatcaagag aagtgtagtg
  5881 gaagctatgc aacgccaagc tcggaaaatg tgcaattacg acaaaatctt ggccacaaag
  5941 aaaaacctag accatgtcaa taaaatctta aaagccaaaa aacttcaaag gcaggccagg
  6001 acagggaata actttgtgaa acgtaggcca ggtcgacctc ggaaatgtcc ccttcaggct
  6061 gtcgtatcaa tgcaagcatt ccaggctgct cagtttgtca acccagaatt gaacagagac
  6121 gaggaaggag cagcactgca cctcagtcct gacacagtta cagatgtaat tgaggctgtt
  6181 gttcagagtg taaatctgaa cccagaacat aaaaaggggt tgaagagaaa aggttggcta
  6241 ttggaagaac agaccagaaa aaagcagaag ccattaccag aggaagaaga gcaagagaat
  6301 aataaaagct ttaatgaagc accagttgag attcccagtc cttctgaaac cccagctaaa
  6361 ccttctgaac ctgaaagtac cttgcagcct gtgctttctc tcatcccaag ggaaaagaag
  6421 cccccacgtc ccccaaagaa gaagtatcag aaagcagggc tgtattctga cgtttacaaa
  6481 actacagacc caaagagtcg attgatccaa ttaaagaaag agaagctgga gtatactcca
  6541 ggagagcatg aatatggatt atttccagcg cccattcatg ttggaaagta tctaagacaa
  6601 aagagaattg acttccagct tccttatgat atcctttggc agtggaaaca caatcagcta
  6661 tacaaaaagc cagatgtccc actatataag aaaattcgtt caaatgtcta cgttgatgtc
  6721 aaaccccttt ctggttacga agctaccacc tgtaactgta agaagccaga tgatgacacc
  6781 aggaagggct gtgttgatga ctgcctcaat agaatgatct ttgctgagtg ttcccccaac
  6841 acttgcccat gtggcgagca atgctgtaac cagaggatac agaggcatga atgggtgcaa
  6901 tgtctagaac gatttcgagc tgaggaaaaa ggttggggaa tcagaaccaa agagccccta
  6961 aaagctgggc agttcatcat tgaataccta ggggaggtcg tcagtgaaca ggagttcagg
  7021 aacaggatga ttgagcagta tcataatcac agtgaccact actgcctgaa cctggatagt
  7081 gggatggtga ttgacagtta ccgcatggga aatgaggccc gattcatcaa ccatagctgt
  7141 gacccaaatt gtgaaatgca gaaatggtct gttaatggag tataccggat tggactctat
  7201 gctcttaaag acatgccagc tgggactgaa ctcacttatg attataactt tcattccttc
  7261 aatgtggaaa aacagcaact ttgtaagtgt ggctttgaga aatgtcgagg aatcatcgga
  7321 ggcaagagtc agcgtgtgaa tggactcacc agcagcaaaa acagccagcc catggccaca
  7381 cacaaaaaat ctggacggtc aaaagagaag agaaagtcta agcacaagct gaagaaaagg
  7441 agaggccatc tctctgagga acccagtgaa aatatcaaca ccccaactag attgaccccc
  7501 caattacaga tgaagccaat gtccaatcgt gaaaggaact ttgtgttaaa gcatcatgta
  7561 ttcttggtcc gaaactggga gaagattcgt caaaaacagg aggaagtaaa gcacaccagt
  7621 gataatattc actcagcatc attatatacc cgttggaatg ggatctgccg agatgatggg
  7681 aatatcaagt ctgatgtctt catgacccag ttctctgccc tgcagacagc tcgatctgtt
  7741 cgaacaagac ggttggcagc tgcagaggaa aatattgaag tggctcgggc agcccgccta
  7801 gcccagatct tcaaagaaat ttgtgatggt atcatctctt ataaagattc ttcccggcaa
  7861 gcactggcag ctccactttt gaaccttccc ccaaagaaaa agaatgctga ttattatgag
  7921 aagatctctg atcccctaga tcttatcacc atagagaagc agatcctcac tggttactat
  7981 aagacagtgg aagcttttga tgctgacatg ctcaaagtct ttcggaatgc tgagaagtac
  8041 tatgggcgta aatccccagt tgggagagat gtttgtcgtc tacgaaaggc ctattacaat
  8101 gcccggcatg aggcatcagc ccagattgat gagattgtgg gagagacagc aagtgaggca
  8161 gacagcagtg agacctcagt ctctgaaaag gagaatgggc atgagaagga cgacgatgtt
  8221 attcgctgta tctgtggcct ctacaaggat gaaggtctca tgatccagtg tgacaagtgc
  8281 atggtatggc agcactgtga ttgtatggga gtgaactcag atgtggagca ctacctttgt
  8341 gagcagtgtg acccaaggcc tgtggacagg gaggttccca tgatccctcg gccccactat
  8401 gcccaacctg gctgtgtcta cttcatctgt ttgctccgag atgacttgct gcttcgtcag
  8461 ggtgactgtg tgtatctgat gagggatagt cggcgcaccc ctgatggcca cccggtccgt
  8521 cagtcctatc gactgttatc tcacattaac cgagataaac ttgacatctt tcgcattgag
  8581 aagctttgga agaatgaaaa agaggaacgg tttgcctttg gtcaccatta tttccgtccc
  8641 cacgaaacac accactctcc atcccgtcgg ttctatcata atgaactatt tcgggtgcca
  8701 ctctatgaga tcattccctt ggaggctgta gtggggacct gctgtgtgtt ggacctttat
  8761 acgtattgta aagggagacc caaaggagta aaggagcaag atgtgtacat ctgtgattat
  8821 cggcttgaca agtcagcaca cctgttttac aagatccacc ggaaccgcta tcctgtctgc
  8881 accaaaccct atgcttttga tcacttcccc aagaagctca ctcccaaaaa agatttctcg
  8941 cctcattacg tcccagacaa ctacaagagg aatggaggac gatcatcctg gaagtctgag
  9001 cgctcaaagc cacccctaaa agacttgggc caggaggatg atgctctacc cttgattgaa
  9061 gaggttctag ccagtcaaga gcaagcagcc aatgagatac ccagcctgga ggagccagaa
  9121 cgggaagggg ccactgctaa cgtcagtgag ggtgaaaaaa aaacagagga aagtagtcaa
  9181 gaaccccagt caacctgtac ccctgaggaa cgacggcata accaacggga acgactcaac
  9241 cagatcttgc tcaatctcct tgaaaaaatc cctggaaaaa atgccattga tgtgacctac
  9301 ttgctggagg aaggatcagg caggaaactg cgaaggcgta ctttgtttat cccagaaaac
  9361 agctttcgaa agtgaccctc aaagaatgag aacctcaagc atctgggatc cagtggagct
  9421 aatcagtcct gcctcctgct ctctgggtat agacaggggt gggaagggtc catctgggca
  9481 aggggaatgg ggccatgttg ttgacattag gtacttaata agccttggag ctagtggaga
  9541 gggagaggaa agggttctgt ccaagacagt tcaggttaat taattttctt ctccattgct
  9601 tcaccttaag ggttaataat gtagagagga gggaggacca cattgatgac cagaacctac
  9661 tggtacttta tagcatttgc cccaccccac agcttaggtt tttctgtcat cctcagatcc
  9721 cacaggcatt gcgaagaagc tgcttcctat acccaggtat aactcaaaat ccaaagggat
  9781 agggccagga tccctattcc taccccatct attctctgtt ggctccaaga gctaccccag
  9841 agaccttaaa cagaaacagt agctgaggct tcttcctaga tacctgacta gggaagtttg
  9901 tctctccttt cttgcccaac caggtcaaag taaaatgtga gttgacagct caaagcactt
  9961 gtaactgctg ccccctccct acctctactc cccaaaatgg aatcatggga tagggaaggc
10021 ccccatgggg tcagaagggc acggtagttc ttgcaattat ttttgtttta cccttcataa
10081 cctgtcaaac atattttttt ctaatgagaa agccaggccc ccgccagcac acatgctgtt
10141 tttaatgcgc tgtagttctt gtgtgtctgc tgtgctgtgc aaatggagat tcagttcaaa
10201 ataaaatcat ttaaaaacct acataaaaag aactctaaac ccacccctgc aacaaaagtc
10261 actacataaa ctgttcagca gtattcacct atcagagtat ttgttgtgag tatagattat
10321 caattgaaaa cactactctt gttttcttaa ttgtacagtt ttcaatgtcc ctttcttaaa
10381 gagacagtat atttctcttc acccctagcc catcttccct caccctcctg aatgacatca
10441 ggaggtatat ccagggtgtc tccttccttc ctactctctt gaccagaagt taacagacta
10501 tactgtctct ttaaaaataa aatttaaaaa gctttgttgt cttttcagac atacatatgc
10561 atatatgttt tagatgttct tataagagaa aagatggttt ttaaatgtgc caagttgtgt
10621 gtgtgtgtgt atatatatgt gtgtatgtgt gtgtatatat atatgtgtgt gtgtatatat
10681 atacacacac acacacacac ctgctgtgtg attggtaagc aatacaatag taaacatgtc
10741 cccattactt ttttctaata ttggaccaat gctgtcctaa ttgtacattt ccccttatgg
10801 tgacgatgct ctgactcgtt taggtagaca cattgaccac cttccattcc attaaatatt
10861 ttttcctttt tcccctttct gtgtcattct tgaggaaaaa acaaaagaga gaggggatgc
10921 caatgatccc cttgagcaga gaaaaagcaa aataaatatt ttattaaaga aaaaagagaa
10981 ttaagaaaat agtttggagt attttcttac tgtagagaag cactgtacat tactaagaga
11041 cctgggtata agatactcac atgtggagct ggaaaaatcg catgtccaag cccgtttgag
11101 tggtttcttt tgtttttcat tgcagggagt gggtgggagg gaggtgggac taggggcact
11161 ttgggggtct ccttttagtc aaaagcgaga aaatgacaag aaagagatta aaattcaatg
11221 tttcctttat agtgttaaac actaaaattt taaaaaagat gaaaaagaaa aaaaaacttt
11281 gtaaaatgcg agaacagaag caaaagacac tacgctctgt cattttatct ttcttttgtt
11341 gaaagactaa aaaaaaactg aaatgttttt tagacaatca aatgttaggt aagtgcaaaa
11401 acttgttttt tcttactggt gtagaaatta atgccttttt ttatttttca gttattttat
11461 aataacgaaa taaaaagaac cccccagctg ccaggcgggt tttggtgttt gaaatgcggg
11521 gcaaagcact acatcactgc aaatagatac agagttagtc tgcatgtctg taggctgtgt
11581 gattgcggaa aatataaatg ctgctaatat atttcctttt tacaaaagca tatctaaata
11641 gatgattgtt ttgatgttaa tctttgtaaa ttatgtatta ccaattttaa cattggatgt
11701 aattgcatac aaagcttgca tctcaatcct tgaaagtcta gtattaaatg gaaaaaactt
11761 ttcctaactg tggaaaaaaa aaaa
SMARCA2 (accession No. NM_003070):
(SEQ ID NO: 143)
        1 gcgtcttccg gcgcccgcgg aggaggcgag ggtgggacgc tgggcggagc ccgagtttag
      61 gaagaggagg ggacggctgt catcaatgaa gtcatattca taatctagtc ctctctccct
    121 ctgtttctgt actctgggtg actcagagag ggaagagatt cagccagcac actcctcgcg
    181 agcaagcatt actctactga ctggcagaga caggagaggt agatgtccac gcccacagac
    241 cctggtgcga tgccccaccc agggccttcg ccggggcctg ggccttcccc tgggccaatt
    301 cttgggccta gtccaggacc aggaccatcc ccaggttccg tccacagcat gatggggcca
    361 agtcctggac ctccaagtgt ctcccatcct atgccgacga tggggtccac agacttccca
    421 caggaaggca tgcatcaaat gcataagccc atcgatggta tacatgacaa ggggattgta
    481 gaagacatcc attgtggatc catgaagggc actggtatgc gaccacctca cccaggcatg
    541 ggccctcccc agagtccaat ggatcaacac agccaaggtt atatgtcacc acacccatct
    601 ccattaggag ccccagagca cgtctccagc cctatgtctg gaggaggccc aactccacct
    661 cagatgccac caagccagcc gggggccctc atcccaggtg atccgcaggc catgagccag
    721 cccaacagag gtccctcacc tttcagtcct gtccagctgc atcagcttcg agctcagatt
    781 ttagcttata aaatgctggc ccgaggccag cccctccccg aaacgctgca gcttgcagtc
    841 caggggaaaa ggacgttgcc tggcttgcag caacaacagc agcagcaaca gcagcagcag
    901 cagcagcagc agcagcagca gcagcagcaa cagcagccgc agcagcagcc gccgcaacca
    961 cagacgcagc aacaacagca gccggccctt gttaactaca acagaccatc tggcccgggg
  1021 ccggagctga gcggcccgag caccccgcag aagctgccgg tgcccgcgcc cggcggccgg
  1081 ccctcgcccg cgccccccgc agccgcgcag ccgcccgcgg ccgcagtgcc cgggccctca
  1141 gtgccgcagc cggccccggg gcagccctcg cccgtcctcc agctgcagca gaagcagagc
  1201 cgcatcagcc ccatccagaa accgcaaggc ctggaccccg tggaaattct gcaagagcgg
  1261 gaatacagac ttcaggcccg catagctcat aggatacaag aactggaaaa tctgcctggc
  1321 tctttgccac cagatttaag aaccaaagca accgtggaac taaaagcact tcggttactc
  1381 aatttccagc gtcagctgag acaggaggtg gtggcctgca tgcgcaggga cacgaccctg
  1441 gagacggctc tcaactccaa agcatacaaa cggagcaagc gccagactct gagagaagct
  1501 cgcatgaccg agaagctgga gaagcagcag aagattgagc aggagaggaa acgccgtcag
  1561 aaacaccagg aatacctgaa cagtattttg caacatgcaa aagattttaa ggaatatcat
  1621 cggtctgtgg ccggaaagat ccagaagctc tccaaagcag tggcaacttg gcatgccaac
  1681 actgaaagag agcagaagaa ggagacagag cggattgaaa aggagagaat gcggcgactg
  1741 atggctgaag atgaggaggg ttatagaaaa ctgattgatc aaaagaaaga caggcgttta
  1801 gcttaccttt tgcagcagac cgatgagtat gtagccaatc tgaccaatct ggtttgggag
  1861 cacaagcaag cccaggcagc caaagagaag aagaagagga ggaggaggaa gaagaaggct
  1921 gaggagaatg cagagggtgg ggagtctgcc ctgggaccgg atggagagcc catagatgag
  1981 agcagccaga tgagtgacct ccctgtcaaa gtgactcaca cagaaaccgg caaggttctg
  2041 ttcggaccag aagcacccaa agcaagtcag ctggacgcct ggctggaaat gaatcctggt
  2101 tatgaagttg cccctagatc tgacagtgaa gagagtgatt ctgattatga ggaagaggat
  2161 gaggaagaag agtccagtag gcaggaaacc gaagagaaaa tactcctgga tccaaatagc
  2221 gaagaagttt ctgagaagga tgctaagcag atcattgaga cagctaagca agacgtggat
  2281 gatgaataca gcatgcagta cagtgccagg ggctcccagt cctactacac cgtggctcat
  2341 gccatctcgg agagggtgga gaaacagtct gccctcctaa ttaatgggac cctaaagcat
  2401 taccagctcc agggcctgga atggatggtt tccctgtata ataacaactt gaacggaatc
  2461 ttagccgatg aaatggggct tggaaagacc atacagacca ttgcactcat cacttatctg
  2521 atggagcaca aaagactcaa tggcccctat ctcatcattg ttcccctttc gactctatct
  2581 aactggacat atgaatttga caaatgggct ccttctgtgg tgaagatttc ttacaagggt
  2641 actcctgcca tgcgtcgctc ccttgtcccc cagctacgga gtggcaaatt caatgtcctc
  2701 ttgactactt atgagtatat tataaaagac aagcacattc ttgcaaagat tcggtggaaa
  2761 tacatgatag tggacgaagg ccaccgaatg aagaatcacc actgcaagct gactcaggtc
  2821 ttgaacactc actatgtggc ccccagaagg atcctcttga ctgggacccc gctgcagaat
  2881 aagctccctg aactctgggc cctcctcaac ttcctcctcc caacaatttt taagagctgc
  2941 agcacatttg aacaatggtt caatgctcca tttgccatga ctggtgaaag ggtggactta
  3001 aatgaagaag aaactatatt gatcatcagg cgtctacata aggtgttaag accattttta
  3061 ctaaggagac tgaagaaaga agttgaatcc cagcttcccg aaaaagtgga atatgtgatc
  3121 aagtgtgaca tgtcagctct gcagaagatt ctgtatcgcc atatgcaagc caaggggatc
  3181 cttctcacag atggttctga gaaagataag aaggggaaag gaggtgctaa gacacttatg
  3241 aacactatta tgcagttgag aaaaatctgc aaccacccat atatgtttca gcacattgag
  3301 gaatcctttg ctgaacacct aggctattca aatggggtca tcaatggggc tgaactgtat
  3361 cgggcctcag ggaagtttga gctgcttgat cgtattctgc caaaattgag agcgactaat
  3421 caccgagtgc tgcttttctg ccagatgaca tctctcatga ccatcatgga ggattatttt
  3481 gcttttcgga acttccttta cctacgcctt gatggcacca ccaagtctga agatcgtgct
  3541 gctttgctga agaaattcaa tgaacctgga tcccagtatt tcattttctt gctgagcaca
  3601 agagctggtg gcctgggctt aaatcttcag gcagctgata cagtggtcat ctttgacagc
  3661 gactggaatc ctcatcagga tctgcaggcc caagaccgag ctcaccgcat cgggcagcag
  3721 aacgaggtcc gggtactgag gctctgtacc gtgaacagcg tggaggaaaa gatcctcgcg
  3781 gccgcaaaat acaagctgaa cgtggatcag aaagtgatcc aggcgggcat gtttgaccaa
  3841 aagtcttcaa gccacgagcg gagggcattc ctgcaggcca tcttggagca tgaggaggaa
  3901 aatgaggaag aagatgaagt accggacgat gagactctga accaaatgat tgctcgacga
  3961 gaagaagaat ttgacctttt tatgcggatg gacatggacc ggcggaggga agatgcccgg
  4021 aacccgaaac ggaagccccg tttaatggag gaggatgagc tgccctcctg gatcattaag
  4081 gatgacgctg aagtagaaag gctcacctgt gaagaagagg aggagaaaat atttgggagg
  4141 gggtcccgcc agcgccgtga cgtggactac agtgacgccc tcacggagaa gcagtggcta
  4201 agggccatcg aagacggcaa tttggaggaa atggaagagg aagtacggct taagaagcga
  4261 aaaagacgaa gaaatgtgga taaagatcct gcaaaagaag atgtggaaaa agctaagaag
  4321 agaagaggcc gccctcccgc tgagaaactg tcaccaaatc cccccaaact gacaaagcag
  4381 atgaacgcta tcatcgatac tgtgataaac tacaaagata ggtgtaacgt ggagaaggtg
  4441 cccagtaatt ctcagttgga aatagaagga aacagttcag ggcgacagct cagtgaagtc
  4501 ttcattcagt taccttcaag gaaagaatta ccagaatact atgaattaat taggaagcca
  4561 gtggatttca aaaaaataaa ggaaaggatt cgtaatcata agtaccggag cctaggcgac
  4621 ctggagaagg atgtcatgct tctctgtcac aacgctcaga cgttcaacct ggagggatcc
  4681 cagatctatg aagactccat cgtcttacag tcagtgttta agagtgcccg gcagaaaatt
  4741 gccaaagagg aagagagtga ggatgaaagc aatgaagagg aggaagagga agatgaagaa
  4801 gagtcagagt ccgaggcaaa atcagtcaag gtgaaaatta agctcaataa aaaagatgac
  4861 aaaggccggg acaaagggaa aggcaagaaa aggccaaatc gaggaaaagc caaacctgta
  4921 gtgagcgatt ttgacagcga tgaggagcag gatgaacgtg aacagtcaga aggaagtggg
  4981 acggatgatg agtgatcagt atggaccttt ttccttggta gaactgaatt ccttcctccc
  5041 ctgtctcatt tctacccagt gagttcattt gtcatatagg cactgggttg tttctatatc
  5101 atcatcgtct ataaactagc tttaggatag tgccagacaa acatatgata tcatggtgta
  5161 aaaaacacac acatacacaa atatttgtaa catattgtga ccaaatgggc ctcaaagatt
  5221 cagattgaaa caaacaaaaa gcttttgatg gaaaatatgt gggtggatag tatatttcta
  5281 tgggtgggtc taatttggta acggtttgat tgtgcctggt tttatcacct gttcagatga
  5341 gaagattttt gtcttttgta gcactgataa ccaggagaag ccattaaaag ccactggtta
  5401 ttttattttt catcaggcaa ttttcgaggt ttttatttgt tcggtattgt ttttttacac
  5461 tgtggtacat ataagcaact ttaataggtg ataaatgtac agtagttaga tttcacctgc
  5521 atatacattt ttccatttta tgctctatga tctgaacaaa agctttttga attgtataag
  5581 atttatgtct actgtaaaca ttgcttaatt tttttgctct tgatttaaaa aaaagttttg
  5641 ttgaaagcgc tattgaatat tgcaatctat atagtgtatt ggatggcttc ttttgtcacc
  5701 ctgatctcct atgttaccaa tgtgtatcgt ctccttctcc ctaaagtgta cttaatcttt
  5761 gctttctttg cacaatgtct ttggttgcaa gtcataagcc tgaggcaaat aaaattccag
  5821 taatttcgaa gaatgtggtg ttggtgcttt cctaataaag aaataattta gcttgacaaa
  5881 aaaaaaaaaa aa
SMARCA4 (accession No. NM_001128844):
(SEQ ID NO: 144)
        1 ggagaggccg ccgcggtgct gagggggagg ggagccggcg agcgcgcgcg cagcgggggc
      61 gcgggtggcg cgcgtgtgtg tgaagggggg gcggtggccg aggcgggcgg gcgcgcgcgc
    121 gaggcttccc ctcgtttggc ggcggcggcg gcttctttgt ttcgtgaaga gaagcgagac
    181 gcccattctg cccccggccc cgcgcggagg ggcgggggag gcgccgggaa gtcgacggcg
    241 ccggcggctc ctgcgtctcg cccttttgcc caggctagag tgcagtggtg cggtcatggt
    301 tcactgcagc ctcaacctcc tggactcagc aggaggccac tgtctgcagc tcccgtgaag
    361 atgtccactc cagacccacc cctgggcgga actcctcggc caggtccttc cccgggccct
    421 ggcccttccc ctggagccat gctgggccct agcccgggtc cctcgccggg ctccgcccac
    481 agcatgatgg ggcccagccc agggccgccc tcagcaggac accccatccc cacccagggg
    541 cctggagggt accctcagga caacatgcac cagatgcaca agcccatgga gtccatgcat
    601 gagaagggca tgtcggacga cccgcgctac aaccagatga aaggaatggg gatgcggtca
    661 gggggccatg ctgggatggg gcccccgccc agccccatgg accagcactc ccaaggttac
    721 ccctcgcccc tgggtggctc tgagcatgcc tctagtccag ttccagccag tggcccgtct
    781 tcggggcccc agatgtcttc cgggccagga ggtgccccgc tggatggtgc tgacccccag
    841 gccttggggc agcagaaccg gggcccaacc ccatttaacc agaaccagct gcaccagctc
    901 agagctcaga tcatggccta caagatgctg gccagggggc agcccctccc cgaccacctg
    961 cagatggcgg tgcagggcaa gcggccgatg cccgggatgc agcagcagat gccaacgcta
  1021 cctccaccct cggtgtccgc aacaggaccc ggccctggcc ctggccctgg ccccggcccg
  1081 ggtcccggcc cggcacctcc aaattacagc aggcctcatg gtatgggagg gcccaacatg
  1141 cctcccccag gaccctcggg cgtgcccccc gggatgccag gccagcctcc tggagggcct
  1201 cccaagccct ggcctgaagg acccatggcg aatgctgctg cccccacgag cacccctcag
  1261 aagctgattc ccccgcagcc aacgggccgc ccttcccccg cgccccctgc cgtcccaccc
  1321 gccgcctcgc ccgtgatgcc accgcagacc cagtcccccg ggcagccggc ccagcccgcg
  1381 cccatggtgc cactgcacca gaagcagagc cgcatcaccc ccatccagaa gccgcggggc
  1441 ctcgaccctg tggagatcct gcaggagcgc gagtacaggc tgcaggctcg catcgcacac
  1501 cgaattcagg aacttgaaaa ccttcccggg tccctggccg gggatttgcg aaccaaagcg
  1561 accattgagc tcaaggccct caggctgctg aacttccaga ggcagctgcg ccaggaggtg
  1621 gtggtgtgca tgcggaggga cacagcgctg gagacagccc tcaatgctaa ggcctacaag
  1681 cgcagcaagc gccagtccct gcgcgaggcc cgcatcactg agaagctgga gaagcagcag
  1741 aagatcgagc aggagcgcaa gcgccggcag aagcaccagg aatacctcaa tagcattctc
  1801 cagcatgcca aggatttcaa ggaatatcac agatccgtca caggcaaaat ccagaagctg
  1861 accaaggcag tggccacgta ccatgccaac acggagcggg agcagaagaa agagaacgag
  1921 cggatcgaga aggagcgcat gcggaggctc atggctgaag atgaggaggg gtaccgcaag
  1981 ctcatcgacc agaagaagga caagcgcctg gcctacctct tgcagcagac agacgagtac
  2041 gtggctaacc tcacggagct ggtgcggcag cacaaggctg cccaggtcgc caaggagaaa
  2101 aagaagaaaa agaaaaagaa gaaggcagaa aatgcagaag gacagacgcc tgccattggg
  2161 ccggatggcg agcctctgga cgagaccagc cagatgagcg acctcccggt gaaggtgatc
  2221 cacgtggaga gtgggaagat cctcacaggc acagatgccc ccaaagccgg gcagctggag
  2281 gcctggctcg agatgaaccc ggggtatgaa gtagctccga ggtctgatag tgaagaaagt
  2341 ggctcagaag aagaggaaga ggaggaggag gaagagcagc cgcaggcagc acagcctccc
  2401 accctgcccg tggaggagaa gaagaagatt ccagatccag acagcgatga cgtctctgag
  2461 gtggacgcgc ggcacatcat tgagaatgcc aagcaagatg tcgatgatga atatggcgtg
  2521 tcccaggccc ttgcacgtgg cctgcagtcc tactatgccg tggcccatgc tgtcactgag
  2581 agagtggaca agcagtcagc gcttatggtc aatggtgtcc tcaaacagta ccagatcaaa
  2641 ggtttggagt ggctggtgtc cctgtacaac aacaacctga acggcatcct ggccgacgag
  2701 atgggcctgg ggaagaccat ccagaccatc gcgctcatca cgtacctcat ggagcacaaa
  2761 cgcatcaatg ggcccttcct catcatcgtg cctctctcaa cgctgtccaa ctgggcgtac
  2821 gagtttgaca agtgggcccc ctccgtggtg aaggtgtctt acaagggatc cccagcagca
  2881 agacgggcct ttgtccccca gctccggagt gggaagttca acgtcttgct gacgacgtac
  2941 gagtacatca tcaaagacaa gcacatcctc gccaagatcc gttggaagta catgattgtg
  3001 gacgaaggtc accgcatgaa gaaccaccac tgcaagctga cgcaggtgct caacacgcac
  3061 tatgtggcac cccgccgcct gctgctgacg ggcacaccgc tgcagaacaa gcttcccgag
  3121 ctctgggcgc tgctcaactt cctgctgccc accatcttca agagctgcag caccttcgag
  3181 cagtggttta acgcaccctt tgccatgacc ggggaaaagg tggacctgaa tgaggaggaa
  3241 accattctca tcatccggcg tctccacaaa gtgctgcggc ccttcttgct ccgacgactc
  3301 aagaaggaag tcgaggccca gttgcccgaa aaggtggagt acgtcatcaa gtgcgacatg
  3361 tctgcgctgc agcgagtgct ctaccgccac atgcaggcca agggcgtgct gctgactgat
  3421 ggctccgaga aggacaagaa gggcaaaggc ggcaccaaga ccctgatgaa caccatcatg
  3481 cagctgcgga agatctgcaa ccacccctac atgttccagc acatcgagga gtccttttcc
  3541 gagcacttgg ggttcactgg cggcattgtc caagggctgg acctgtaccg agcctcgggt
  3601 aaatttgagc ttcttgatag aattcttccc aaactccgag caaccaacca caaagtgctg
  3661 ctgttctgcc aaatgacctc cctcatgacc atcatggaag attactttgc gtatcgcggc
  3721 tttaaatacc tcaggcttga tggaaccacg aaggcggagg accggggcat gctgctgaaa
  3781 accttcaacg agcccggctc tgagtacttc atcttcctgc tcagcacccg ggctgggggg
  3841 ctcggcctga acctccagtc ggcagacact gtgatcattt ttgacagcga ctggaatcct
  3901 caccaggacc tgcaagcgca ggaccgagcc caccgcatcg ggcagcagaa cgaggtgcgt
  3961 gtgctccgcc tctgcaccgt caacagcgtg gaggagaaga tcctagctgc agccaagtac
  4021 aagctcaacg tggaccagaa ggtgatccag gccggcatgt tcgaccagaa gtcctccagc
  4081 catgagcggc gcgccttcct gcaggccatc ctggagcacg aggagcagga tgagagcaga
  4141 cactgcagca cgggcagcgg cagtgccagc ttcgcccaca ctgcccctcc gccagcgggc
  4201 gtcaaccccg acttggagga gccacctcta aaggaggaag acgaggtgcc cgacgacgag
  4261 accgtcaacc agatgatcgc ccggcacgag gaggagtttg atctgttcat gcgcatggac
  4321 ctggaccgca ggcgcgagga ggcccgcaac cccaagcgga agccgcgcct catggaggag
  4381 gacgagctcc cctcgtggat catcaaggac gacgcggagg tggagcggct gacctgtgag
  4441 gaggaggagg agaagatgtt cggccgtggc tcccgccacc gcaaggaggt ggactacagc
  4501 gactcactga cggagaagca gtggctcaag gccatcgagg agggcacgct ggaggagatc
  4561 gaagaggagg tccggcagaa gaaatcatca cggaagcgca agcgagacag cgacgccggc
  4621 tcctccaccc cgaccaccag cacccgcagc cgcgacaagg acgacgagag caagaagcag
  4681 aagaagcgcg ggcggccgcc tgccgagaaa ctctccccta acccacccaa cctcaccaag
  4741 aagatgaaga agattgtgga tgccgtgatc aagtacaagg acagcagcag tggacgtcag
  4801 ctcagcgagg tcttcatcca gctgccctcg cgaaaggagc tgcccgagta ctacgagctc
  4861 atccgcaagc ccgtggactt caagaagata aaggagcgca ttcgcaacca caagtaccgc
  4921 agcctcaacg acctagagaa ggacgtcatg ctcctgtgcc agaacgcaca gaccttcaac
  4981 ctggagggct ccctgatcta tgaagactcc atcgtcttgc agtcggtctt caccagcgtg
  5041 cggcagaaaa tcgagaagga ggatgacagt gaaggcgagg agagtgagga ggaggaagag
  5101 ggcgaggagg aaggctccga atccgaatct cggtccgtca aagtgaagat caagcttggc
  5161 cggaaggaga aggcacagga ccggctgaag ggcggccggc ggcggccgag ccgagggtcc
  5221 cgagccaagc cggtcgtgag tgacgatgac agtgaggagg aacaagagga ggaccgctca
  5281 ggaagtggca gcgaagaaga ctgagccccg acattccagt ctcgaccccg agcccctcgt
  5341 tccagagctg agatggcata ggccttagca gtaacgggta gcagcagatg tagtttcaga
  5401 cttggagtaa aactgtataa acaaaagaat cttccatatt tatacagcag agaagctgta
  5461 ggactgtttg tgactggccc tgtcctggca tcagtagcat ctgtaacagc attaactgtc
  5521 ttaaagagag agagagagaa ttccgaattg gggaacacac gatacctgtt tttcttttcc
  5581 gttgctggca gtactgttgc gccgcagttt ggagtcactg tagttaagtg tggatgcatg
  5641 tgcgtcaccg tccactcctc ctactgtatt ttattggaca ggtcagactc gccgggggcc
  5701 cggcgagggt atgtcagtgt cactggatgt caaacagtaa taaattaaac caacaacaaa
  5761 acgcacagcc aaaaaaaaa
BPTF (accession No. NM_182641):
(SEQ ID NO: 145)
        1 cgccccccct gcgcccgccc ctcccccttc gctttccttc tccccccgcc tcggctccga
      61 catgaggggc cggcggggca ggccgcccaa gcagcccgcg gctcccgctg cggagcgctg
    121 cgccccggcc ccgccgccac cgccgccgcc gcccacgtcc ggacccatcg gggggctccg
    181 ctcgcggcac cgcggcagca gccggggcag gtgggccgcc gcccaggctg aggtggcgcc
    241 caagacgcgg ctgagctcgc ccaggggggg cagcagtagc cggaggaagc cgccgccgcc
    301 gccgccggcc ccccccagca ccagcgcccc gggccggggg gggcgaggag gcgggggcgg
    361 caggacgggg ggcgggggcg gcggcggcca cctggcccgg accaccgcgg cccggagggc
    421 cgtcaacaaa gtggtgtacg atgaccacga gagcgaggag gaggaggaag aggaggacat
    481 ggtctccgag gaggaggagg aggaggacgg cgacgccgag gagacccagg attctgagga
    541 cgacgaggag gatgagatgg aagaggacga cgatgactcc gattatccgg aggagatgga
    601 agacgacgac gacgacgcca gttactgcac ggaaagcagc ttcaggagcc atagtaccta
    661 cagcagcact ccaggtaggc gaaaaccaag agtacatcgg cctcgttctc ctatattgga
    721 agaaaaagac atcccgcccc ttgaatttcc caagtcctct gaggatttaa tggtgcctaa
    781 tgagcatata atgaatgtca ttgccattta cgaggtactg cggaactttg gcactgtttt
    841 gagattatct ccttttcgct ttgaggactt ttgtgcagct ctggtgagcc aagagcagtg
    901 cacactcatg gcagagatgc atgttgtgct tttgaaagca gttctgcgtg aagaagacac
    961 ttccaatact acctttggac ctgctgatct gaaagatagc gttaattcca cactgtattt
  1021 catagatggg atgacgtggc cagaggtgct gcgggtgtac tgtgagagtg ataaggagta
  1081 ccatcacgtt cttccttacc aagaggcaga ggactaccca tatggaccag tagagaacaa
  1141 gatcaaagtt ctacagtttc tagtcgatca gtttcttaca acaaatattg ctcgagagga
  1201 attgatgtct gaaggggtga tacagtatga tgaccattgt agggtttgtc acaaacttgg
  1261 ggatttgctt tgctgtgaga catgttcagc agtataccat ttggaatgtg tgaagccacc
  1321 tcttgaggag gtgccagagg acgagtggca gtgtgaagtc tgtgtagcac acaaggtgcc
  1381 tggtgtgact gactgtgttg ctgaaatcca aaaaaataaa ccatatattc gacatgaacc
  1441 tattggatat gatagaagtc ggaggaaata ctggttcttg aaccgaagac tcataataga
  1501 agaagataca gaaaatgaaa atgaaaagaa aatttggtat tacagcacaa aggtccaact
  1561 tgcagaatta attgactgtc tagacaaaga ttattgggaa gcagaactct gcaaaattct
  1621 agaagaaatg cgtgaagaaa tccaccgaca catggacata actgaagacc tgaccaataa
  1681 ggctcggggc agtaacaaat cctttctggc ggcagctaat gaagaaattt tggaatccat
  1741 aagagccaaa aagggagaca ttgataatgt taaaagccca gaagaaacag aaaaagacaa
  1801 gaatgagact gagaatgact ctaaagatgc tgagaaaaac agagaagaat ttgaagacca
  1861 gtcccttgaa aaagacagtg acgacaaaac accagatgat gaccctgagc aaggaaaatc
  1921 tgaggtaggt gatttcaaat cggagaagtc caacggggag ctaagtgaat ctcctggagc
  1981 tggaaaagga gcatctggct caactcgaat catcaccaga ttgcggaatc cagatagcaa
  2041 acttagtcag ctgaagagcc agcaggtggc agccgctgca catgaagcaa ataaattatt
  2101 taaggagggc aaagaggtac tggtagttaa ctctcaagga gaaatttcac ggttgagcac
  2161 caaaaaggaa gtgatcatga aaggaaatat caacaattat tttaaattgg gtcaagaagg
  2221 gaagtatcgc gtctaccaca atcaatactc caccaattca tttgctttga ataagcacca
  2281 gcacagagaa gaccatgata agagaaggca tcttgcacat aagttctgtc tgactccagc
  2341 aggagagttc aaatggaacg gttctgtcca tgggtccaaa gttcttacca tatctactct
  2401 gagactgact atcacccaat tagaaaacaa catcccttca tcctttcttc atcccaactg
  2461 ggcatcacat agggcaaatt ggatcaaggc agttcagatg tgtagcaaac ccagagaatt
  2521 tgcattggct ttagccattt tggagtgtgc agttaaacca gttgtgatgc taccaatatg
  2581 gcgagaatct ttaggacata ccaggttaca ccggatgaca tcaattgaaa gagaagaaaa
  2641 ggagaaagtc aaaaaaaaag agaagaaaca ggaagaagaa gaaacgatgc agcaagcgac
  2701 atgggtaaaa tacacatttc cagttaagca tcaggtttgg aaacaaaaag gtgaagagta
  2761 cagagtgaca ggatatggtg gttggagctg gattagtaaa actcatgttt ataggtttgt
  2821 tcctaaattg ccaggcaata ctaatgtgaa ttacagaaag tcgttagaag gaaccaaaaa
  2881 taatatggat gaaaatatgg atgagtcaga taaaagaaaa tgttcacgaa gtccaaaaaa
  2941 aataaaaata gagcctgatt ctgaaaaaga tgaggtaaaa ggttcagatg ctgcaaaagg
  3001 agcagaccaa aatgaaatgg atatctcaaa gattactgag aagaaggacc aagatgtgaa
  3061 ggagctctta gattctgaca gtgataaacc ctgcaaggaa gaaccaatgg aagtagacga
  3121 tgacatgaaa acagagtcac atgtaaattg tcaggagagt tctcaagtag atgtggtcaa
  3181 tgttagtgag ggttttcatc taaggactag ttacaaaaag aaaacaaaat catccaaact
  3241 agatggactt cttgaaagga gaattaaaca gtttacactg gaagaaaaac agcgactcga
  3301 aaaaatcaag ttggagggtg gaattaaggg tataggaaag acttctacaa attcttcaaa
  3361 aaatctctct gaatcaccag taataacgaa agcaaaagaa gggtgtcaga gtgactcgat
  3421 gagacaagaa cagagcccaa atgcaaataa tgatcaacct gaggacttga ttcagggatg
  3481 ttcagaaagt gattcctcag ttcttagaat gagtgatcct agtcatacca caaacaaact
  3541 ttatccaaaa gatcgagtgt tagatgatgt ctccattcgg agcccagaaa caaaatgtcc
  3601 gaaacaaaat tccattgaaa atgacataga agaaaaagtc tctgaccttg ccagtagagg
  3661 ccaggaaccc agtaagagta aaacaaaagg aaatgatttt ttcatcgatg actctaaact
  3721 agccagtgca gatgatattg gtactttgat ctgtaagaac aaaaaaccgc tcatacagga
  3781 ggaaagtgac accattgttt cttcttccaa gagtgcttta cattcatcag tgcctaaaag
  3841 taccaatgac agagatgcca cacctctgtc aagagcaatg gactttgaag gaaaactggg
  3901 atgtgactct gaatctaata gcactttgga aaatagttct gataccgtgt ctattcagga
  3961 tagcagtgaa gaagatatga ttgttcagaa tagcaatgaa agcatttctg aacagttcag
  4021 aactcgagaa caagatgttg aagtcttgga gccgttaaag tgtgagttgg tttctggtga
  4081 gtccactgga aactgtgagg acaggctgcc ggtcaagggg actgaagcaa atggtaaaaa
  4141 accaagtcag cagaagaaat tagaggagag accagttaat aaatgtagtg atcaaataaa
  4201 gctaaaaaat accactgaca aaaagaataa tgaaaatcga gagtctgaaa agaaaggaca
  4261 gagaacaagt acatttcaaa taaatggaaa agataataaa cccaaaatat atttgaaagg
  4321 tgaatgcttg aaagaaattt ctgagagtag agtagtaagt ggtaatgttg aaccaaaggt
  4381 taataatata aataaaataa tccctgagaa tgatattaaa tcattgactg ttaaagaatc
  4441 tgctataagg ccattcatta atggtgatgt catcatggaa gattttaatg aaagaaacag
  4501 ctccgaaaca aaatcgcatt tgctgagttc ttcagatgct gaaggtaact accgagatag
  4561 ccttgagacc ctgccatcaa ccaaagagtc tgacagtaca cagacgacca caccctcagc
  4621 atcttgtcca gaaagcaatt cagttaatca ggtagaagat atggaaatag aaacctcaga
  4681 agttaagaaa gttacttcat cacctattac ttctgaagag gaatctaatc tcagtaatga
  4741 ctttattgat gaaaatggtc tgcccatcaa caaaaatgaa aatgtcaatg gagaatctaa
  4801 aagaaaaacc gtcatcacag aagtcaccac gatgacctcc acagtggcca cagaatcaaa
  4861 aactgtgatc aaggtagaaa aaggcgataa gcaaactgtg gtttcttcca cagaaaattg
  4921 tgcaaaatcc actgtcacaa ccaccactac aacagtgacc aagctttcca caccctccac
  4981 aggcggcagt gtggacatca tctctgtaaa ggagcagagc aaaaccgtgg tcaccacgac
  5041 agtgacagac tccctgacca ccacgggagg cacactggtt acatctatga ctgtgagcaa
  5101 agagtattcc acacgagaca aagtgaaact gatgaaattt tcaagaccaa agaagactcg
  5161 ttcaggtaca gctctgccat cctatagaaa atttgttacc aagagcagca agaagagcat
  5221 ttttgttttg cctaatgatg acttaaaaaa gttggcccga aaaggaggaa tccgagaggt
  5281 cccttatttt aattacaatg caaaacctgc tttggatata tggccatatc cttctcctag
  5341 accgaccttt ggcatcactt ggaggtatag acttcagaca gtaaagtcct tagctggagt
  5401 gagcctgatg ttacggttac tgtgggcaag tttgagatgg gatgatatgg cggccaaggc
  5461 tcctccagga ggagggacta cacggacaga aacatccgaa actgaaatca caacaacaga
  5521 aataattaag aggagagatg ttggtcctta tggcattcga tctgaatatt gtatcaggaa
  5581 aatcatttgt cccattggag ttccagaaac accaaaagaa acgcctacac ctcagaggaa
  5641 aggccttcga tcaagtgcac tgcggccaaa gagaccagaa acgcccaagc aaactggccc
  5701 tgttattatt gaaacctggg tagcagaaga agaactggaa ttgtgggaga tcagggcatt
  5761 tgctgagaga gtggagaaag aaaaggcaca agcagttgag caacaggcta agaaacgact
  5821 ggagcagcag aagccgacag tgattgcaac ttccactact tccccaacaa gcagtacaac
  5881 cagcaccatc tctccagcac agaaggttat ggtggccccc ataagtggct cagttacaac
  5941 tggaaccaaa atggtactaa ctactaaagt tggatctcca gctacagtaa cattccaaca
  6001 aaacaagaac tttcatcaaa cctttgctac atgggttaag caaggccagt caaattcagg
  6061 cgttgttcaa gtacagcaga aagtcctggg tatcattcca tcaagtacag gtaccagtca
  6121 gcaaaccttt acttcattcc agcccaggac agcaacagtc acaattaggc ccaatacctc
  6181 aggctctgga ggaaccacaa gcaattcaca agtaatcaca gggcctcaga ttcgccctgg
  6241 tatgaccgtg attagaacac cactccaaca gtcaacacta ggaaaggcaa ttattcgaac
  6301 acctgtgatg gtacagccag gtgctcctca gcaagtgatg actcaaatca tcagggggca
  6361 gcctgtctcc actgcagtct ccgcccctaa cacggtttcc tcaacacctg ggcagaaaag
  6421 cttaacttca gcaacgtcca cttcaaatat acagtcttca gcctcacaac cccctcgccc
  6481 ccaacaagga caagtgaagc tcaccatggc tcaacttact cagttaacac agggccacgg
  6541 tggcaatcaa ggtttgacag tagtaattca aggacaaggt caaactactg gacagttgca
  6601 gttgatacct caaggggtga ctgtactccc aggcccaggc cagcagctaa tgcaagctgc
  6661 aatgccaaat ggtactgttc agcgattcct ctttacccca ttggcaacaa cagccaccac
  6721 agccagcacc accaccacca ctgtttccac gacagcagca ggtacaggtg aacaaaggca
  6781 gagtaaactg tcaccccaga tgcaggtaca tcaagacaaa accctgccac cagctcagtc
  6841 atcaagtgtg ggtccagcag aagcccagcc acagactgct cagccttcag ctcagcccca
  6901 gccccaaacc cagccccagt ccccagctca gcctgaagtt cagactcagc ctgaagttca
  6961 gacccaaaca actgtttcat cccatgtccc ttctgaagca caacccaccc acgcacagtc
  7021 atccaagccc caagttgcag cacagtctca gcctcaaagt aatgtccaag gacagtctcc
  7081 tgttcgtgtc caaagtccat cacagactcg aatacgtcca tcaactccat cccaactgtc
  7141 tcctggacaa caatcccagg ttcagactac aacctcacaa ccgattccaa ttcaaccaca
  7201 tacatctctt cagatacctt cccaaggcca gccacagtca caaccccagg tacagtcttc
  7261 aactcaaact ctttcatcag gacaaacttt aaatcaagtt actgtttcat ccccatcccg
  7321 tcctcagcta caaatacagc agccacagcc ccaagtcatt gctgtgcctc agctgcaaca
  7381 acaagtccag gttctctctc agatccagtc acaggttgtg gctcagatac aggctcagca
  7441 aagtggtgtg ccccagcaaa tcaaactcca gttacctatc caaattcagc aaagcagtgc
  7501 tgtgcagact caccagattc agaatgtggt tacagtgcag gcagccagtg tgcaagagca
  7561 gttgcaaagg gttcagcaac tcagggatca gcagcaaaag aagaaacagc aacagataga
  7621 aattaagcgt gaacacaccc tccaagcttc taatcaaagt gaaatcattc agaaacaggt
  7681 ggtgatgaag cataatgctg taatagaaca tttaaaacag aaaaagagca tgactccagc
  7741 tgaaagagaa gagaatcaaa gaatgattgt ctgtaaccag gtgatgaagt atattttgga
  7801 taagatagat aaagaagaaa aacaggcagc aaaaaaacgg aagcgtgaag agagtgtgga
  7861 gcagaaacgt agcaagcaga atgccactaa gctgtcagct ctgctcttca agcacaaaga
  7921 gcagctcaga gccgagatcc tgaagaagag agcactcctg gacaaggatc tgcaaattga
  7981 agtgcaggaa gagctgaaga gagacctgaa aattaagaaa gaaaaagacc tgatgcagtt
  8041 ggctcaggcc acagcagtag ctgcaccctg ccccccagtg acaccagctc ctccagcccc
  8101 tccagcccct ccaccttcac ctccccctcc acctgctgtg caacacacag gccttctgtc
  8161 cacgcccacc ttacctgctg cttcccagaa gaggaagcgg gaagaggaaa aagactccag
  8221 ctcaaagtcc aagaaaaaga aaatgatctc tactacctca aaggaaacta agaaggacac
  8281 aaagctttac tgtatctgta aaacgcctta tgatgaatct aaattttata ttggctgtga
  8341 tcggtgtcag aattggtacc atgggcgctg cgttggcatc ttgcaaagtg aggcagagct
  8401 cattgatgag tatgtctgtc cacagtgcca gtcaacagag gatgccatga cagtgctcac
  8461 gccactaaca gagaaggatt atgaggggtt gaagagggtg ctccgttcct tacaggccca
  8521 taagatggcc tggcctttcc ttgaaccagt agaccctaat gatgcaccag attattatgg
  8581 tgttattaag gaacctatgg accttgccac catggaagaa agagtacaaa gacgatatta
  8641 tgaaaagctg acggaatttg tggcagatat gaccaaaatt tttgataact gtcgttacta
  8701 caatccaagt gactccccat tttaccagtg tgcagaagtt ctcgaatcat tctttgtaca
  8761 gaaattgaaa ggcttcaaag ctagcaggtc tcataacaac aaactgcagt ctacagcttc
  8821 ttaaagttca gcgtgttaac ctaacataaa acacagcaag aatctggttg tctgaactat
  8881 tttaaattaa ggagccagat gtttttagtc aggctatcct gacaagactt gacctaaact
  8941 tcgtttttat tggtcataac agtccaatta tattcttggc caattttgtc caacggacaa
  9001 gaaaaaagca aagtcaacga caccattatc ttgtcaagat cagatggttt tactattgtg
  9061 gcagaagcga gaaaactttg tttattgaaa aaaaaagaaa aagaaagcaa gaaaaaaaga
  9121 tactatgggg tcaagtgtaa ctccatggaa atgccacgtc tgctcttcag tgaagaagct
  9181 ggtttagagt ctcacagaaa acttttgact gtatttattt attgttgcaa aaaagacgct
  9241 tttttattgc tgccctcatt tgtcagctaa ttattttttc ttataaaatc cagccccggt
  9301 tacatataat catctgtatc ttatcatgat tcctgtaggt aaaagtacaa gacgacctct
  9361 agatgtcttt tctttctatg aaaggagctg ctatgtacac atgtgcacac acacacaact
  9421 gggaatcaac aatgagttta ttgttcatgg tagattaaaa ttaagcttgc ataaaggttg
  9481 ggctaagtgg tcctggacta cagactctgt tgccttgaat ataacagtac aatttgtcaa
  9541 ttactctgca ccaggctaaa atgagtaaaa tctatttgaa ggtatcttgt ttgtaaacat
  9601 ttgtcagatt ctaatttttt tcttttgtat taaaattcaa ctatggatgt atatgaaaca
  9661 aaataaatgg agataatttt tctcccacag acagaggtgt ctttgaatgt gcgctaatga
  9721 ttatctgtaa gcctttgtgg ggagggaggc ctgcaaggtc atgaaaggca gaagagtcta
  9781 attgtgcctg gatttctcca ggacagcagt ggcccctcgt tttatcattc ccagtccatt
  9841 gtcatcacgt cagagaaaaa tcttcagggg tgctaatcct gttgcatcag ttgatcatac
  9901 taacgagaac ggtaatgcga caagatacac attgccttca tctgtacatt ctgtgatacc
  9961 aggcaaatta ccaattacac acagctactt atattttatg aagggcattt tttagatgac
10021 ctcatcctct gtgttatttg ttgattgggt ttgttttctg tttgttggtt tgtttgtttc
10081 ttccacgtaa ggaaaagtag tgtaaacagt agcgagaaaa tggaaaccac agaggaagat
10141 gtattttgca tgtttttcct ttcagtgttc ttacacgttg tatcactgca ttgtggtaat
10201 agcttctata aaatctgcca tagttggatt atgcagcttt gcaaaaattt ttactagatt
10261 ttgcactaac tcatattagc tttgtcctac caacttctgg aatttatcta attattgttt
10321 ttcaaagttt ctttcctttt aatgtttccc tgctatgcaa aacctttccc agacctcagt
10381 ttcttaaaag aaagatgttg ctacagttcc cgattctttc ttattacagg ctcaggtgta
10441 caggttattc tgggttaatt ttatctaatg aagcccattc ctttttgtac ataaagatgt
10501 cacttaaact tatgcttaca aactaaagac taatcgctca atatgaaaac atgaaaaaat
10561 ttttgcttaa agtattaaga tggaagtagt taaatatggg ttattttgtc cttttacttt
10621 tttaaaaaat gttacatatt gtatgcactg tgctgatgca agaattctac attttaatga
10681 gttataaaat tattctgcat ctcatcacgt cacagtattt ctgtactatt tattcatata
10741 tataaatata tatgggctta atcatttaaa atttgttgca gcaagaactt tcctacctgt
10801 aggcaataga ttgctatgtt tttaacaaat tgtggcaaat tctaaacagc aattcttttg
10861 tacgtaatag gacatttcat cctagaaaaa taaagtaatg tttttgacat tgga
SMARCA1 (accession No. NM_001282874):
(SEQ ID NO: 146)
        1 ggcctgagcg aaggggttgg aagcggagtg attccccacc cctgctccat ctagctcttt
      61 ccagtgcagc cactgccgcc gcccaggagc cctcgtcccc tgccttgtcc ccctactcgt
    121 tcccgctccc acggcatgga gcaggacact gccgcagtgg cagccaccgt ggcagccgcg
    181 gatgcgaccg ccactatcgt ggtcatagag gacgagcagc ccgggccgtc cacctctcag
    241 gaggagggag cggccgccgc ggccaccgaa gccaccgcgg ccacggagaa gggcgagaag
    301 aagaaggaga aaaacgtttc ttcatttcaa ctcaaacttg ctgctaaagc gcctaaatct
    361 gaaaaggaaa tggacccaga atatgaagag aaaatgaaag ccgaccgagc aaagagattt
    421 gaatttttac tgaagcagac agaacttttt gcacatttca ttcagccttc agcacagaaa
    481 tctccaacat ctccactgaa catgaaattg ggacgtcccc gaataaagaa agatgaaaag
    541 cagagcttaa tttctgctgg agactaccgc cataggcgca cagagcaaga agaagatgaa
    601 gagctactgt ctgagagtcg gaaaacatct aatgtgtgta ttagatttga ggtgtcacct
    661 tcatatgtga aaggggggcc actgagagat tatcagattc gaggactgaa ttggttgatc
    721 tctttatatg aaaatggagt caatggcatt ttggctgatg aaatgggcct tgggaaaact
    781 ttacaaacaa ttgctttgct tggttacctg aaacactacc gaaatattcc tggacctcac
    841 atggttttag ttccaaagtc tactttacac aactggatga atgaatttaa acgatgggtc
    901 ccatctctcc gtgtcatttg ttttgtcgga gacaaggatg ccagagctgc ttttattcgt
    961 gatgaaatga tgccaggaga gtgggatgtt tgcgttactt cttatgagat ggtaattaaa
  1021 gaaaaatctg tattcaaaaa gtttcactgg cgatacctgg tcattgatga agctcacaga
  1081 ataaagaatg aaaaatctaa gctttcagag attgttcgtg agttcaagtc gactaaccgc
  1141 ttgctcctaa ctggaacacc tttgcagaat aacctgcatg aactgtgggc cttactcaac
  1201 tttttattgc ctgatgtctt taattctgca gatgactttg attcttggtt tgacactaaa
  1261 aattgtcttg gtgatcaaaa actcgtggaa agacttcatg cagttttaaa accatttttg
  1321 ttacgccgta taaaaactga tgtagagaag agtctgccac ctaaaaagga aataaagatt
  1381 tacttggggc tgagtaagat gcaacgagaa tggtatacaa aaatcctgat gaaagatatt
  1441 gatgttttaa actcttctgg caagatggac aagatgcgac tcttaaacat tctgatgcag
  1501 cttcgaaagt gttgtaatca tccatatctg tttgatggtg ctgaacctgg tccaccttat
  1561 accactgatg agcatattgt cagcaacagt ggtaaaatgg tagttctgga taaactattg
  1621 gccaaactca aagaacaggg ttcaagggtt ctcattttca gccagatgac tcgcttgctg
  1681 gatattttgg aagattattg catgtggcgt ggttatgagt attgtcgact ggatggacaa
  1741 accccgcatg aagaaagaga ggataaattc ctagaagtgg aatttctggg tcaaagggaa
  1801 gcaatagagg cttttaatgc tcctaatagt agcaaattca tctttatgct aagtaccagg
  1861 gctggaggtc tcggaattaa cctggcaagt gctgatgtgg ttatactata tgattcagac
  1921 tggaacccac aggttgatct acaagctatg gatcgagcac atcgtattgg tcagaagaaa
  1981 ccagtacgtg tattccgtct catcactgac aacactgttg aagagaggat tgtagaaaga
  2041 gctgagataa aactgagact cgattcaatt gttatacaac aaggaagact cattgaccaa
  2101 cagtctaaca agctggcaaa agaggaaatg ttacaaatga tacggcatgg agccacccat
  2161 gtttttgctt ctaaagagag tgagttgaca gatgaagaca ttacaactat tctggaaaga
  2221 ggggaaaaga agactgcaga gatgaatgaa cgcctgcaaa aaatgggaga gtcttctcta
  2281 agaaatttta gaatggacat tgaacaaagt ttatacaaat ttgagggaga agattataga
  2341 gaaaaacaga agcttggcat ggtggaatgg attgaacctc ctaaacgaga acgcaaagca
  2401 aactacgcag tggatgccta ctttagagag gctttgcgtg tcagcgagcc aaagattcca
  2461 aaggctccac ggcctccaaa acagccaaat gttcaggatt ttcaattttt cccaccacgc
  2521 ttatttgagc tcctggaaaa ggaaattctt tattatcgga agacaatagg ctataaggtt
  2581 ccaaggaatc ctgatatccc aaatccagct ctggctcaaa gagaagagca aaaaaagatt
  2641 gatggagctg aacctcttac accagaagag actgaagaaa aggaaaaact tctcacacaa
  2701 ggtttcacaa actggactaa acgagatttt aaccagttta ttaaagctaa tgagaaatat
  2761 ggaagagatg acattgataa catagctcga gaggtagagg gcaaatcccc tgaggaggtc
  2821 atggagtatt cagctgtatt ttgggaacgt tgcaatgaat tacaggacat tgagaaaatt
  2881 atggctcaaa ttgaacgtgg agaagcaaga attcaacgaa ggatcagtat caagaaagcc
  2941 ctggatgcca aaattgcaag atacaaggct ccatttcatc agttgcgcat tcagtatgga
  3001 accagcaaag gaaagaacta tactgaggaa gaagatagat tcttgatttg tatgttacac
  3061 aaaatgggct ttgatagaga aaatgtatat gaagaattaa gacagtgtgt acgaaatgct
  3121 ccccagttta gatttgactg gtttatcaag tctaggactg ccatggaatt ccagagacgc
  3181 tgtaacactc tgatttcatt gattgagaaa gaaaatatgg aaattgagga aagagagaga
  3241 gcagaaaaga agaaacgggc aactaaaact ccaatgtcac agaaaagaaa agcagagtca
  3301 gctactgaga gctctggaaa gaaggatgtc aagaaggtga aatcctaaag cctagaaata
  3361 aagttttaaa tgggaaactg ctattttctt gttcccatct tcaaatgcta attgccagtt
  3421 ccagtgtatt catggtactc taagaaaaat ctctttggtt ttgatttctt gcatatttta
  3481 tatattttac aatgctttct acctgaaatg tgtagcttta tattttatgg cattctagta
  3541 tttttgtgta ctgtattttg tgcatttcat gtcttcatca aaatcctctc agtccttgtt
  3601 cttttgaagc ttgtgctgag gttttagctt ttctatgttt tatatgccgc tgctttgaaa
  3661 gagaacctag attctatagt tgtattattg ttgtttcata ctttaaattt atatggctgt
  3721 ggaaaaacga attaaaatgt tttgaggaga aagacttttt cacttctttg ttgctttctt
  3781 ttctattgag tctgggcttg tttgtgttac tgcatactgt gattagcata ataattgttt
  3841 ctttgaggtc atctaaatat ttttttccta aaggaataaa gggtgaggaa agaaaaatat
  3901 taaaaaagct aatatttgat actgtgcttg ctgtcagtat gcattacatt taaattattc
  3961 tctattcaag tgggaaaata taataaagaa atgtctataa gaaatttaaa aaaaaaaaaa
  4021 aaaaa
```

In some embodiments the therapeutic peptide to be expressed by the bacterial cell is caspase, such caspase 3 (for example, expressed in its activated form), or NIPP1.

### IV. Cancer Treatment

Bacteria such as Salmonella, Clostridium and Bifidobacterium have a natural tropism for cancers, such as solid tumors. Types of cancer that can be treated using the methods of the invention include, but are not limited to, solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma).

In some aspects, the subject is treated with radiation and chemotherapy before, after or during administration of the bacterial cells described herein.

### V. Administration

The invention includes administration of the attenuated Salmonella strains described herein and methods for preparing pharmaceutical compositions and administering such as well. Such methods comprise formulating a pharmaceutically acceptable carrier with one or more of the attenuated Salmonella strains described herein.

A pharmaceutical composition of the invention is formulated to be compatible with its intended route of administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

For intravenous administration, suitable carriers include physiological saline, bacteriostatic water, Cremophor EL™ (BASF; Parsippany, N.J.) or phosphate buffered saline (PBS). It must be stable under the conditions of manufacture and storage and must be preserved against the contaminating action of other (undesired) microorganisms. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Injectable solutions can be prepared by incorporating the active compound in the required amount in an appropriate solvent with one or a combination of ingredients discussed above. Generally, dispersions are prepared by incorporating the active compound into a vehicle which contains a basic dispersion medium and various other ingredients discussed above. In the case of powders for the preparation of injectable solutions, the preferred methods of preparation are vacuum drying and freeze-drying which yields a powder of the active ingredient plus any additional desired ingredient from a previously.

Oral compositions generally include an inert diluent or an edible carrier. For example, they can be enclosed in gelatin capsules. For the purpose of oral therapeutic administration, the active compound can be incorporated with excipients and used in the form of tablets, troches, or capsules.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the bacteria are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the bacteria are formulated into ointments, salves, gels, or creams as generally known in the art.

It is especially advantageous to formulate compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

When administered to a patient the attenuated Salmonella can be used alone or may be combined with any physiological carrier. In general, the dosage ranges from about 1.0 c.f.u./kg to about 1×10¹² c.f.u./kg; optionally from about 1.0 c.f.u./kg to about 1×10¹⁰ c.f.u./kg; optionally from about 1.0 c.f.u./kg to about 1×10⁸ c.f.u./kg; optionally from about 1×10² c.f.u./kg to about 1×10⁸ c.f.u./kg; optionally from about 1×10⁴ c.f.u./kg to about 1×10⁸ c.f.u./kg; optionally from about 1×10⁵ c.f.u./kg to about 1×10¹² c.f.u./kg; optionally from about 1×10⁵ c.f.u./kg to about 1×10¹⁰ c.f.u./kg; optionally from about 1×10⁵ c.f.u./kg to about 1×10⁸ c.f.u./kg.

### EXAMPLES

The following examples are provided in order to demonstrate and further illustrate certain embodiments and aspects of the present invention and are not to be construed as limiting the scope thereof.

### Example I

### Introduction

Delivering protein drugs into the cytoplasm of cancer cells would expand the number of treatable cancer targets. More than 60% of the pathways that control cellular function are intracellular (1) and almost all are difficult to access. Intracellular pathways control most of the hallmarks of cancer (2) and have been the focus of a significant fraction of cancer research. Because of their specificity, protein biologics are excellent candidates for interfering with these pathways. However, bringing functional proteins across the cell membrane is technically challenging. Effective intracellular delivery, coupled with specific protein drugs, has the potential to provide new treatments for previously incurable cancers.

### Materials and Methods

### Bacterial Cultures

All bacterial cultures (both Salmonella and DH5α) were grown in LB (10 g/L sodium chloride, 10 g/L tryptone and 5 g/L yeast extract). Resistant strains of bacteria were grown in the presence of carbenicllin (100 μg/ml), chloramphenicol (33 μg/ml), kanamycin (50 μg/ml) and/or 100 μg/ml of DAP.

### Bacterial Strains and Plasmid Construction

Fifteen strains of Salmonella Enterica serovar Typhimurium were used throughout the experiments (Table S1). All plasmids contained a ColE1 origin and either chloramphenicol or ampicillin resistance (Table S2). All assembled DNA constructs were transformed into chemically competent DH5a E. Coli (New England Biolabs, Ipswich, MA) before electroporation into Salmonella. All cloning reagents, buffer reagents, and primers were from New England Biolabs, Fisher Scientific (Hampton, NH), and Invitrogen, (Carlsbad, CA), respectively, unless otherwise noted.

For electroporation, Salmonella cultures were grown to an optical density between 0.6 and 0.8, washed twice with 25 ml of ice-cold water, and resuspended in 400 μl ice cold water. DNA (200 ng for plasmids and 1-2 μg for linear DNA) was mixed with 50 μl of the bacterial suspension and electroporated in a 1 mm electroporation cuvette at 1,800V and 25 μF with a time constant of 5 msec.

The parental control strain (Par) was based on an attenuated therapeutic strain of Salmonella (VNP20009) that has three deletions, ΔmsbB, ΔpurI, and Δxyl that eliminate most toxicities in vivo. To enable balanced-lethal plasmid retention a strain was used (VNP200010) that has the asd gene deleted (1). A second strain (ΔflhD Par) was the basis for many strains in the study (Table S1). This strain was generated by first deleting flhD, then asd.

Genetic deletions were created using a modified lambda red recombination protocol (2). Salmonella were transformed with pkd46 (Yale CGSC E. Coli stock center) and grown from a single colony in 50 ml of LB. At an optical density of 0.1, arabinose was added to the bacterial cultures to a final concentration of 20 mM. When the optical density reached between 0.6 and 0.8, bacteria were centrifuged at 3000×g and washed twice with 25 ml ice-cold, ultrapure water (Millipore). The pelleted Salmonella were resuspended in 400 μl ice-cold water. A linear DNA segment was designed to insert an in-frame deletion into the gene (here flhD). It was generated by PCR amplification of FRT-KAN-FRT from plasmid pkd4 using primers vr121 and vr309 (Table S3). This PCR product contained kanamycin resistance flanked by FRT recombination sites and 50 base pair regions homologous to flhD. After electroporation, Salmonella recovered in LB for 2 hours at 37° C. and were left overnight at room temperature. This recovery solution was plated on kanamycin (50 μg/ml) agar plates and incubated at 37° C. until colonies formed. Colonies were screened for knockouts by colony PCR. Successful transformants were plated on kanamycin plates and grown overnight at 43° C. to eliminate pkd46 from the bacteria.

A similar process was used to delete asd. Transformants with successful deletion of flhD, were transformed with pkd46. A PCR product was created to insert an in-frame deletion into asd by PCR amplifying FRT-CHLOR-FRT from plasmid pkd3 using primers vr266 and vr268 (Table S3). This PCR product contained chloramphenicol resistance flanked by FRT recombination sites and 50 base pair regions homologous to asd. During recovery, electroporated bacteria were plated on agar containing 33 μg/ml chloramphenicol and 100 μg/ml diaminopimelic acid (DAP). Successful transformants were grown in the presence of chloramphenicol, kanamycin and DAP.

To generate the intracellular reporting strain of Salmonella, parental Salmonella strain (Par) was transformed with a plasmid containing PsseJ-GFP (plasmid P1; Table S2). The construction of this plasmid was initiated by first creating a promoter-less-GFP plasmid from pLacGFP and pQS-GFP [1]. The pQS-GFP plasmid contains chloramphenicol resistance, the ColE1 origin of replication, and the asd gene. Expression of ASD is necessary in Δasd strains and creates a balanced lethal system that maintains gene expression in vivo. The Plac-GFP gene circuit was amplified from plasmid pLacGFP with primers nd1 and nd2 (Table S4). The PCR product and the plasmid were digested with Aat2 and Pci1 and ligated with T4 DNA ligase (NEB, catalog #M0202S). The PsseJ promoter was amplified from the genome of SL1344 Salmonella using primers nd3 and nd4 (Table S4). This PCR product and the backbone plasmid were ligated after digestion with XbaI and Pci1.

A strain that re-expresses flhDC (flhDC Sal, Table S1) was created by transforming ΔflhD Salmonella with plasmid P2 (Table S2). Plasmid P2 was formed from temporary plasmid P3. Plasmid P3 was formed by amplifying flhDC from Salmonella genomic DNA using primers vr46 and vr47 (Table S4) and ligating it into plasmid PBAD-his-mycA (Invitrogen; catalog #V430-01). The PCR product was digested with NcoI, XhoI and DpnI (NEB, catalog #s R0193S, R0146S and R0176L). The PBAD-his-myc plasmid was digested with NcoI and XhoI and treated with calf intestinal phosphatase (NEB, catalog #M0290) for three hours. The PCR product was ligated into the plasmid backbone with T4 DNA ligase (NEB, catalog #M0202S).

The Plac-GFP-myc circuit was inserted into P3 by Gibson Assembly. (1) The insert (Plac-GFP-myc) was amplified from plasmid pLacGFP (1) using primers vr394 and vr395 (Table S4), which added homology regions to the backbone and added the myc tag. (2) The backbone plasmid (P3) was amplified using primers vr385 and vr386, which added homology to the insert. (3) Both PCR products were digested with DpnI for three hours, (4) and ligated by Gibson Assembly (HiFi master mix, NEB, catalog #E2621L). The gene for aspartate semialdehyde dehydrogenase (asd) gene was inserted by Gibson Assembly by amplifying asd from genomic Salmonella DNA using primers vr424 and vr425 and amplifying the plasmid backbone with primers vr426 and vr427.

A strain that re-expresses flhDC and produces GFP after invasion (flhDC reporting, Table S1) was created by transforming ΔflhD Salmonella with plasmid P4 (Table S2). The PsseJ-GFP-myc genetic circuit was amplified from P1 using primers vr269 and vr270, and the backbone of plasmid P3 was amplified using primers vr271 and vr272. The two PCR products were ligated by Gibson Assembly.

To generate the PsifA intracellular promoter-reporter strain, the PsifA promoter was cloned from Salmonella genomic DNA using primers nd5 and nd6 and inserted into P1 using XbaI and Pci1 creating plasmid P5. The PsifA reporter strain was created by transforming plasmid P5 into background Salmonella by electroporation. The generation of the PsseJ reporter strain is described above. To investigate lysis in Salmonella, lysis gene E (LysE) was put under control of PBAD. LysE was cloned using primers nd7 and nd8 and inserted into pBAD/Myc-His A (Invitrogen) using NcoI and KpnI to form plasmid P6.

Intracellular delivering (ID) Salmonella were created by cloning the Lysin E gene behind the PsseJ promoter. LysE was amplified using primers nd9 and nd10 and cloned into P1 using XbaI and Aat2. The Plac-GFP circuit was added to this plasmid by cloning it from plasmid pLacGFP using primers nd 11 and nd12 and inserting using SacI to create plasmid P7. This plasmid constitutively expresses myc-tagged GFP to identify bacteria in both live-cell and fixed-cell assays.

Genomic knockouts ΔsifA and ΔsseJ were created using the modified lambda red recombination protocol described in the creation of ΔflhD Salmonella above. Salmonella were transformed with pkd46. Linear DNA with homologous flanking regions was produced by PCR of plasmid pkd4 using primers vr432 and vr433 for ΔsseJ; and vr434 and vr435 for ΔsifA. After electroporation and recovery, colonies were screened for knockouts by colony PCR of the junction sites of the inserted PCR amplified products. Successful transformants were plated on kanamycin plates (50 μg/ml) and grown overnight at 43° C. to remove pkd46.

ID Salmonella that re-expresses flhDC (flhDC-ID Sal) was created by transforming ΔflhD with plasmids P8. Plasmid P8 was created by amplifying the Pssej-LysE gene circuit from P7 using primers vr398 and vr399 and ligating it into plasmid P2 using Gibson Assembly. The P2 backbone plasmid was amplified using primers vr396 and vr397.

A strain of ID Salmonella that constitutively expresses luciferase (ID Sal-luc; Table S1) was created by cloning Plac-luc from pMA3160 (Addgene) using primers ch1 and ch2. The P7 plasmid backbone was amplified with primers ch3 and ch4 and the pieces were ligated by Gibson Assembly to form plasmid P9 (Table S2).

To create ID Salmonella that express anti-b-actin nanobody (NB), PBAD inducible nanobody was cloned in place of flhDC in plasmid P8. The actin nanobody (Chromotek, catalog #acr) was amplified using primers vr466 and vr467. The delivery plasmid backbone was amplified using primers vr448 and vr449. The two PCR products were ligated by Gibson Assembly to create plasmid P10.

To create ID Salmonella that express the central domain of NIPP1 (NIPP1-CD), NIPP1-CD was cloned into plasmid pLacGFP. NIPP1-CD and the backbone plasmid were amplified using primers nd13-nd16 ligated by Gibson Assembly. The pLac-NIPP1-CD circuit was cloned using primers nd11 and nd17 (Table S4) and inserted into P7 using SacI to create plasmid P11.

To create ID Salmonella that intracellularly deliver CT caspase-3 (CT Casp-3), parental Salmonella were transformed with plasmid P12. This plasmid was created by PCR amplifying template DNA encoding for CT caspase-3 using primers, vr450 and vr451 from the constitutively two-chain (CT) caspase-3 encoding plasmid pC3D175CT. The pC3D175CT plasmid (Hardy Lab DNA archive Box 7, line 62) was constructed similarly to the caspase-6 CT expression construct [3] using Quikchange mutagenesis on a construct encoding full-length human caspase-3 in a pET23 expression vector (Addgene). Plasmid pC3D175CT encodes human caspase-3 residues 1-175, followed by a TAA stop codon, a ribosome binding sequence and the coding sequence for a start methionine and an inserted serine followed by the coding sequence for residues 176-286 with a six-histidine tag appended. The backbone of plasmid P8 was PCR amplified using primers vr448 and vr449 and the PCR products were ligated as previously described.

TABLE S1
Bacterial strains

| | Background/ | | Genetic | |
|---|---|---|---|---|
| Strain | Knockouts | Plasmid | functions | Description |
| Parental (Par) | ΔmsbB, ΔpurI, | — | — | Non-pathogenic therapeutic |
| | Δxyl, Δasd | | | Salmonella; deletion of asd |
| | | | | enables balanced lethal system to |
| | | | | maintain plasmids in vivo |
| ΔflhD | ΔfthD Par | — | — | Parental Salmonella with flhD |
| | | | | deletion; non-motile |
| Intracellular | Par | P1 | PsseJ-GFP | Intracellularly inducible GFP |
| reporting | | | | |
| flhDC Sal | ΔflhD Par | P2 | PBAD-flhDC | Re-expresses flhDC after |
| | | | Plac-GFP | induction with arabinose |
| | | | PBAD-flhDC | Re-expresses flhDC after |
| | | | | induction with arabinose |
| flhDC reporting | ΔfthD Par | P4 | PsseJ-GFP | Intracellularly inducible GFP |
| PsifA | Par | P5 | PsifA-GFP | Expresses GFP after activation of |
| | | | | PsifA promoter |
| PBAD-LysE | Par | P6 | PBAD-LysE | Bacteria lyse after activation with |
| | | | | arabinose |
| | | | PsseJ-LysE | Bacteria lyse after activation of |
| | | | | PsseJ promoter |
| ID Sal | Par | P7 | Plac-GFP | Constitutively expresses GFP |
| | | | | Predominantly accumulates in |
| | | | | the cytoplasm of cells |
| | | | PsseJ-LysE | Lyses after invasion |
| ΔsifA | ΔsifA Par | P7 | Plac-GFP | Constitutively expresses GFP |
| | | | | Predominantly accumulate in |
| | | | | SCVs |
| | | | PsseJ-LysE | Lyses after invasion |
| ΔsseJ | ΔsseJ Par | P7 | Plac-GFP | Constitutively expresses GFP |
| | | | Plac-GFP | Re-expresses flhDC after |
| | | | PBAD-flhDC | induction with arabinose |
| | | | | Lyses after invasion |
| flhDC-ID Sal | ΔflhD Par | P8 | PsseJ-LysE | Constitutively expresses GFP |
| | | | PsseJ-LysE | Bacteria lyse after activation of |
| | | | | PsseJ promoter |
| ID Sal-luc | Par | P9 | Plac-GFP | Constitutively expresses GFP |
| | | | Plac-luc | and luciferase |
| | | | | Lyses after invasion |
| | | | PsseJ-LysE | Controllably expresses nanobody |
| | | | PBAD-nano | against β-actin |
| NB | Par | P10 | Plac-GFP | Constitutively expresses GFP |
| | | | PsseJ-LysE | Lyses after invasion |
| | | | Plac-NIPP1 | Constitutively expresses NIPP1- |
| NIPP1-CD | Par | P11 | Plac-GFP | CD, and GFP |
| | | | PsseJ-LysE | |
| | | | PBAD- | Lyses after invasion |
| | | | Casp3 | Controllably expresses CT Casp |
| CT Casp-3 | Par | P12 | Plac-GFP | 3 Constitutively expresses GFP |

TABLE S2
Plasmids

| | | | Gene | Gene | |
|---|---|---|---|---|---|
| No. | Name | Origin | Maintenance | Circuits | Purpose |
| P1 | Intracellular | ColE1 | Chlor^{a} | PsseJ-GFP | Expresses GFP after cell |
| | reporting | | ASD^{b} | | invasion |
| P2 | flhDC re- | ColE1 | Amp | PBAD-flhDC | Re-expresses flhDC; |
| | expressing | | ASD | Plac-GFP-myc | Constitutively expresses |
| | | | | | GFP |
| P3 | PBAD-flhDC | ColE1 | Amp^{c} | PBAD-flhDC | Used in construction of |
| | | | | | P2 and P2B |
| P4 | flhDC reporting | ColE1 | Amp | PBAD-flhDC | Measures invasion after |
| | | | | PsseJ-GFP-myc | flhDC re-expression |
| P5 | PsifA reporter | ColE1 | Chlor | PsifA-GFP | Expresses GFP after |
| | plasmid | | ASD | | activation of PsifA |
| | | | | | promoter |
| P6 | Inducible lysis | ColE1 | Chlor | PBAD-LysE | Lyses after activation |
| | | | ASD | | with arabinose |
| P7 | Intracellular lysis | ColE1 | Chlor | PsseJ-LysE | Bacteria lyse after |
| | | | ASD | Plac-GFP-myc | invasion; Constitutively |
| | | | | | expresses GFP |
| P8 | Intracellular lysis | ColE1 | AMP | PsseJ-LysE | Lyses after invasion; |
| | and induced | | ASD | PBAD-flhDC | Re-expresses flhDC; |
| | invasion | | | Plac-GFP-myc | Constitutively expresses |
| | | | | | GFP |
| P9 | Luciferase | ColE1 | Chlor | PsseJ-LysE | Bacteria lyse after |
| | | | ASD | Plac-GFP-myc | invasion; Constitutively |
| | | | | Plac-luc | expresses GFP and |
| | | | | | luciferase |
| P10 | Nanobody | ColE1 | AMP | PsseJ-LysE | Lyses after invasion; |
| | | | ASD | PBAD-nano-flag | Expresses flag-tagged |
| | | | | | nanobody against β- |
| | | | | | actin |
| P11 | NIPP1-CD | ColE1 | Chlor | PsseJ-LysE Plac- | Lyses after invasion; |
| | | | ASD | NIPP1 | Expresses NIPP1-CD |
| P12 | CT Casp-3 | ColE1 | AMP | Plac-GFP | Lyses after invasion; |
| | | | ASD | PsseJ-LysE | Constitutively expresses |
| | | | | PBAD-Casp3 | GFP; Expresses CT Casp 3 |

^{a}Chloramphenicol
^{b}ASD (aspartate-semialdehyde dehydrogenase) is an essential enzyme for lysine synthesis and is necessary for the synthesis of peptidoglycan (4). It is the key gene in the balanced lethal system developed by Nakayama et al. (5) to maintain genes in Salmonella after injection in vivo.
^{c}Ampicillin

TABLE S4
Primers used for gene deletions

| Name | Primer sequence | Gene | Template |
|---|---|---|---|
| vr121 | FOFZCACGGGGTGCGGCTACGTCGCACAAA | flhD forward | pkd4 |
| | AATAAAGTTGGTTATTCTGGGTCTTGAGCG | | |
| | ATTGTGTAGGC (SEQ ID NO: 147) | | |
| vr309 | EOEFATCCTGAGTCAAACGGGTGATCGTCT | flhD reverse | pkd4 |
| | GATGATCGTCAAACCGGAAAAATTAGCCAT | | |
| | GGTCCATATGAATATC (SEQ ID NO: 148) | | |
| vr266 | FZEFAAAATGTTGGTTTTATCGGCTGGCGCG | asd forward | pkd3 |
| | GAATGGTCGGCTCTGTTCTGTCTTGAGCGAT | | |
| | TGTGTAGGC (SEQ ID NO: 149) | | |
| vr268 | OZFOGCCAACTGGCGCAGCATTCGACGCAG | asd reverse | pkd3 |
| | CGGCTCGGCGGCGCCCCATAAATTAGCCAT | | |
| | GGTCCATATGAATATC (SEQ ID NO: 150) | | |
| vr432 | FZEOCATTGAGTGTTGGACAGGGTTATTTCA | sseJ forward | pkd4 |
| | CATCATCTATCAGTTCTGAGTCTTGAGCGAT | | |
| | TGTGTAGGC (SEQ ID NO: 151) | | |
| vr433 | ZZFZTCAGTGGAATAATGATGAGCTATAAA | sseJ reverse | pkd4 |
| | ACTTTCTAACATTATGGCAAAATTAGCCAT | | |
| | GGTCCATATGAATATC (SEQ ID NO: 152) | | |
| vr434 | FZEOCGATTACTATAGGGAATGGTTTTTTAA | sifA forward | pkd4 |
| | AAAGTGAAATCCTTACCAAGTCTTGAGCGA | | |
| | TTGTGTAGGC (SEQ ID NO: 153) | | |
| vr435 | ZZFZAAAAAACAACATAAACAGCCGCTTTG | sifA reverse | pkd4 |
| | TTGTTCTGAGCGAACGTGTAAATTAGCCAT | | |
| | GGTCCATATGAATATC (SEQ ID NO: 154) | | |

TABLE S4
Primers used for plasmid construction

| Name | Sequence | Description |
|---|---|---|
| nd1 | AAAAAAACATGTGTGGAATTGTGAGCGGATAAC | PLac-GFP forward |
| | (SEQ ID NO: 155) | |
| nd2 | AAAAAAGACGTCTTATTTGTATAGTTCATCCATGC | PLac-GFP reverse |
| | C (SEQ ID NO: 156) | |
| nd3 | AAAAAAACATGTCACATAAAACACTAGCACTTT | PsseJ forward |
| | (SEQ ID NO: 157) | |
| nd4 | AAAAAATCTAGACCTCCTTACTTTATTAAACAC | PsseJ reverse |
| | (SEQ ID NO: 158) | |
| nd5 | AAAAAAACATGTTATAAGCGATTAATTGCGCAA | PsifA forward |
| | (SEQ ID NO: 159) | |
| nd6 | AAAAAATCTAGATAATCTCACTTATACTGGAGT | PsifA reverse |
| | (SEQ ID NO: 160) | |
| nd7 | AAAAAACCATGGTTTAAGAAGGAGATATACATAT | LysE forward (into |
| | GG (SEQ ID NO: 161) | PBAD) |
| nd8 | AAAAAAGGTACCTCACTCCTTCCGCACGTAATT | LysE reverse (into |
| | (SEQ ID NO: 162) | PBAD) |
| nd9 | AAAAAATCTAGATTTAAGAAGGAGATATACATAT | LysE forward |
| | GG (SEQ ID NO: 163) | |
| nd10 | AAAAAAGACGTCTCACTCCTTCCGCACGTAATT | LysE reverse |
| | (SEQ ID NO: 164) | |
| nd11 | AAAAAA GAGCTC GACTGGAAAGCGGGCAGTGA | Plac-GFP forward |
| | (SEQ ID NO: 165) | |
| nd12 | AAAAAA GAGCTC AAGCTTGCATGCCTGCAGGAG | Plac-GFP reverse |
| | (SEQ ID NO: 166) | |
| nd13 | TTTAAGAAGGAGATATACATATGGGTGGAGAGGA | NIPP1 forward |
| | TGATG (SEQ ID NO: 167) | |
| nd14 | CTTGCATGCCTGCAGGAGATTTACAGATCCTCTTC | NIPP1 forward |
| | TGAGATGAGTTTTTGTTCGTTCCGAAAGCGACCAA | |
| | C (SEQ ID NO: 168) | |
| nd15 | ATGTATATCTCCTTCTTAAATCTAGAGGTC (SEQ ID | pLacGFP backbone |
| | NO: 169) | forward |
| nd16 | GAACAAAAACTCATCTCAGAAGAGGATCTGTAAA | pLacGFP backbone |
| | TCTCCTGCAGGCATGCA (SEQ ID NO: 170) | reverse |
| nd17 | AAAAAAGAGCTCGTTAGCAATTTAACTGTGATAA | Plac-NIPP1-CD |
| | AC (SEQ ID NO: 171) | reverse |
| ch1 | TCAATCTCCTGCAGGCATGCTTTACACTTTATGCT | Luciferase forward |
| | TCCGGCTCGTATAATAAAAAAAAAAAAGGAGGAA | |
| | AAAAAATGGAAGATGCCAAAAACATTAAGAA | |
| | (SEQ ID NO: 172) | |
| ch2 | GGGGCGTAATTTGATATCAAGCTTTACACGGCGA | Luciferase reverse |
| | TCTTGCCG (SEQ ID NO: 173) | |
| ch3 | AGCTTGATATCAAATTACGCCCC (SEQ ID NO: 174) | P6 backbone forward |
| ch4 | GCATGCCTGCAGGAGATTGA (SEQ ID NO: 175) | P6 backbone reverse |
| vr46 | AAAAAACCATGGGTTAATAAAAGGAGGAATATAT | flhDC forward |
| | ATGCATACATCCGAGTTGCTAAAACA (SEQ ID NO: | |
| | 176) | |
| vr47 | AAAAAACTCGAGAAAAATTAAACAGCCTGTTCGA | flhDC reverse |
| | TCTGTTCAT (SEQ ID NO: 177) | |
| vr394 | CCGCATAGTTAAGCCAGTATACATTTACACTTTAT | pLacGFP backbone |
| | GCTTCCGGCTCGTATAATAAAAAAAAAAGGAGGA | forward |
| | AAAAAAATGAGTAAAGGAGAAGAACTTTTCA (SEQ | |
| | ID NO: 178) | |
| vr395 | TCACGTAGCGATAGCGGAGTTACAGATCCTCTTCT | pLacGFP backbone |
| | GAGATGAGTTTTTGTTCTTTGTATAGTTCATCCAT | reverse |
| | GCCAT (SEQ ID NO: 179) | |
| vr385 | CTCCGCTATCGCTACGTGA (SEQ ID NO: 180) | P3 backbone forward |
| vr386 | TGTATACTGGCTTAACTATGCGG (SEQ ID NO: 181) | P3 backbone reverse |
| vr424 | GCTTGTCTGCTCCCGGCATCGTACGTTTTCGTTCC | asd forward |
| | ATTGG (SEQ ID NO: 182) | |
| vr425 | AGACGGTCACAGCTTGTCTGTATCTGCGTTTACTC | asd reverse |
| | CTGTATTAC (SEQ ID NO: 183) | |
| vr426 | ACAGACAAGCTGTGACCGTCT (SEQ ID NO: 184) | backbone forward |
| vr427 | ATGCCGGGAGCAGACAAGC (SEQ ID NO: 185) | backbone reverse |
| vr269 | CGCAGCGAGTCAGTGAGCACATGTCACATAAAAC | Pssej-GFP-myc |
| | ACTAGCACT (SEQ ID NO: 186) | forward |
| vr270 | CGCACAGATGCGTAAGGAGAATTACAGATCCTCT | Pssej-GFP-myc reverse |
| | TCTGAGATGAGTTTTTGTTCTTTGTATAGTTCATCC | |
| | ATGCCATG (SEQ ID NO: 187) | |
| vr271 | GCTCACTGACTCGCTGCG (SEQ ID NO: 188) | P3 backbone reverse |
| vr272 | TTCTCCTTACGCATCTGTGCG (SEQ ID NO: 189) | P3 backbone forward |
| vr398 | ATCTGTGCGGTATTTCACACCACATGTCACATAAA | Pssej-LysE forward |
| | ACACTAGCACT (SEQ ID NO: 190) | |
| vr399 | TACTGAGAGTGCACCATATGCTCACTCCTTCCGCA | Pssej-LysE reverse |
| | CGTAATTT (SEQ ID NO: 191) | |
| vr396 | GCATATGGTGCACTCTCAGTA (SEQ ID NO: 192) | P2 backbone forward |
| vr397 | GGTGTGAAATACCGCACAGAT (SEQ ID NO: 193) | P2 backbone reverse |
| vr466 | GGGCTAACAGGAGGAATTAACCATGGCTCAGGTG | Actin nanobody |
| | CAGCTGG (SEQ ID NO: 194) | forward |
| vr467 | TACCAGCTGCAGATCTCGAGTTACTTGTCGTCATC | Actin nanobody |
| | GTCTTTGTAGTCCATGCTTCTTGAGGAGACGGTGA | reverse |
| | (SEQ ID NO: 195) | |
| vr448 | CTCGAGATCTGCAGCTGGTA (SEQ ID NO: 196) | P8 backbone forward |
| vr449 | GGTTAATTCCTCCTGTTAGCCC (SEQ ID NO: 197) | P8 backbone reverse |
| vr450 | GGGCTAACAGGAGGAATTAACCATGGACTACAAA | N-term FLAG-Casp |
| | GACGATGACGACAAGATGGAGAACACTGAAAACT | forward |
| | CAGTG (SEQ ID NO: 198) | |
| vr451 | TACCAGCTGCAGATCTCGAGTTACAGATCCTCTTC | C-term myc-Casp3 |
| | TGAGATGAGTTTTTGTTCGTGATAAAAATAGAGTT | reverse |
| | CTTTTGTGAG (SEQ ID NO: 199) | |

### Cell Culture

Four cancer cell lines were used: 4T1 murine breast carcinoma cells; Hepa1-6 murine hepatocellular carcinoma cells; MCF7 human breast carcinoma cells and LS174T human colorectal carcinoma cells (ATCC, Manassas, VA). All cancer cells were grown and maintained in Dulbecco's Minimal Eagle Medium (DMEM) containing 3.7 g/L sodium bicarbonate and 10% fetal bovine serum. For microscopy studies, cells were incubated in DMEM with 20 mM HEPES buffering agent and 10% FBS. To generate tumor spheroids, single cell suspensions of LS174T cells were transferred to PMMA-coated cell culture flasks (2 g/L PMMA in 100% ethanol, dried before use).

Salmonella Invasion into Cancer Cells In Vitro

To observe invasion into cancer cells, Salmonella were administered to mouse 4T1 breast cancer cells grown on coverslips using an invasion assay. The cells and bacteria were stained with phalloidin and anti-Salmonella antibodies and imaged with 100× oil immersion microscopy. The general procedures for invasion assays, immunocytochemistry, and microscopy are detailed in the following sections.

### Invasion Assays

For invasion assays, cancer cells were grown on coverslips for fixed-cell imaging or on well plates for live-cell imaging. For fixed imaging, glass coverslips were placed in 12-well plates and sterilized with UV light in a biosafety hood for 20 minutes. Mouse 4T1 or human MCF7 cells were seeded on the coverslips at 40% confluency and incubated overnight in DMEM. Concurrently, Salmonella were grown to an optical density (OD; at 600 nm) of 0.8. After incubation, the Salmonella were added to the 4T1 cultures at a multiplicity of infection (MOI) of 10 and allowed to infect the cells for two hours. After this invasion period, the cultures were washed five times with 1 ml of phosphate buffered saline (PBS) and resuspended in 2 ml of DMEM with 20 mM HEPES, 10% FBS and 50 μg/ml gentamycin. The added gentamycin removes extracellular bacteria. After six hours of incubation, the media was removed, and the coverslips were fixed with 10% formalin in PBS for 10 minutes.

A similar procedure was used for live-cell imaging. Cells were grown directly on well plates in DMEM (3.7 g/L sodium bicarbonate, 10% FBS) to a confluency between 30 and 50%. After growth to OD 0.8, Salmonella were added to the cell cultures at an MOI of 25 for 2 hours. After invasion, the cancer cells were washed five times with PBS, and 2 ml of DMEM with 50 μg/ml gentamycin was added to each well. Cells and bacteria were directly imaged microscopically.

### Immunocytochemistry

Immunocytochemistry was used to obtain detailed images of Salmonella invaded into cancer cells grown on coverslips. After fixing the coverslips with formalin, they were blocked with staining buffer (PBS with 0.1% Tween 20, 1 mM EDTA, and 2% bovine serum albumin [BSA]) for 30 minutes. The Tween 20 in this buffer selectively permeabilizes mammalian cell membranes, while leaving bacterial membranes intact.

After permeabilization, coverslips were stained to identify Salmonella, released GFP, vacuolar membranes and/or intracellular f-actin with (1) rabbit anti-Salmonella polyclonal antibody (Abcam, ab35156) or FITC-conjugated rabbit anti-Salmonella polyclonal antibody (Abcam, ab69253) (2) rat anti-myc monoclonal antibody (Chromotek, catalog #9e1-100), (3) rabbit anti-LAMP1 polyclonal antibody (Abcam, catalog #ab24170), and (4) Alexaflor-568-conjugated phalloidin (ThermoFisher, catalog #A12380), respectively. Three different staining combinations were used: (1) Salmonella alone; (2) Salmonella, released GFP and actin; and (3) Salmonella, released GFP and vacuoles.

For Salmonella alone staining (combination 1), coverslips were stained with FITC-conjugated anti-Salmonella antibody at 30° C. for one hour and washed three times with staining buffer.

For Salmonella, released GFP and actin staining (combination 2), coverslips were stained with anti-Salmonella and anti-myc primary antibodies at 30° C. for one hour, and washed twice times with staining buffer. Coverslips were incubated with secondary antibodies at a 1:200 dilution for one hour at 30° C.: Alexaflor-647 chicken anti-rabbit (ThermoFisher, catalog #A21443), Alexaflor-488 donkey anti-rat (ThermoFisher, catalog #A21208), and Alexaflor-568-conjugated phalloidin to identify Salmonella, GFP and intracellular f-actin, respectively.

For Salmonella, released GFP and vacuole staining (combination 3), coverslips were stained sequentially with anti-LAMP1 primary antibodies at 30° C. for one hour, and washed three times with staining buffer. Coverslips were incubated with Alexaflor-647 chicken anti-rabbit secondary antibodies (ThermoFisher, catalog #A21443) at a 1:200 dilution for one hour at 30° C. and washed four times with staining buffer. Coverslips were then stained with FITC-conjugated anti-Salmonella antibody and anti-myc primary antibody; and washed three times with staining buffer. Coverslips were incubated with Alexaflor-568 goat anti-rat secondary antibodies (ThermoFisher, A11077) at a 1:200 dilution for one hour at 30° C. to identify GFP.

After all staining, coverslips were washed three times with staining buffer and mounted to glass slides using 20 μl mountant with DAPI (ProLong Gold Antifade Mountant, ThermoFisher, catalog #P36962). Mounted coverslips were cured overnight at room temperature.

### Microscopy

Samples were imaged on a Zeiss Axio Observer Z.1 microscope. Fixed cells on coverslips were imaged with a 100× oil immersion objective (1.4 NA). Tumor sections were images with 10× and 20× objectives (0.3 and 0.4 NA, respectively). Time lapse fluorescence microscopy of live cells in well plates and tumor-chip devices were housed in a humidified, 37° C. environment and imaged with 5×, 10×, 63× or 100× objectives (0.2, 0.3, 1.4 and 1.4 NA, respectively). Fluorescence images were acquired with either 480/525 or 525/590 excitation/emission filters. All images were background subtracted and contrast was uniformly enhanced. Some image analysis was automated using computational code (MATLAB, Mathworks).

### Intracellular Salmonella in Tumors

To determine the fraction of tumor-colonized Salmonella that are intracellular, BALB/c mice with 4T1 tumors were injected with 2×10⁶ CFU of Intracellular reporting Salmonella (with PsseJ-GFP; Table S1). Ninety-six hours after bacterial injection, mice were sacrificed and tumors were excised, sectioned and stained as described in the Immunohistochemistry section below. Tumor sections were stained to identify Salmonella and GFP, which is produced by intracellular Salmonella. The fraction of intracellular Salmonella was determined by identifying Salmonella (n=1,258) in 8 images and determining the number that co-localize with GFP.

### Immunohistochemistry

Excised tumor sections were fixed in 10% formalin for 3 days. Fixed tumor samples were then stored in 70% ethanol for 1 week. Tumor samples were embedded in paraffin and sectioned into 5 μm sections. Deparaffinization was performed by washing the sectioned tissue three times in 100% xylene, twice in 100% ethanol, once in 95% ethanol, once in 70% ethanol, once in 50% ethanol, and once in DI water. Each wash step was performed for 5 minutes. Antigen retrieval was performed by incubating the tissue sections in 95° C., 20 mM sodium citrate (pH 7.6) buffer for 20 minutes. Samples were left in sodium citrate buffer until the temperature reduced to 40° C. Samples were then rehydrated with two quick (<1 minute) rinses in DI water followed by one five-minute wash in TBS-T.

Prior to staining, tissue sections were blocked with Dako blocking buffer (Dako, catalog #X0909) for one hour. Tissue sections were stained to identify Salmonella and GFP with 1:100 dilutions of (1) FITC-conjugated rabbit anti-Salmonella polyclonal antibody (Abcam, catalog #ab69253), and (2) either rat anti-myc monoclonal antibody (Chromotek, catalog #9e1-100) or rat anti-GFP monoclonal antibody (Chromotek, catalog #3h9-100) in Tris buffered saline with 0.1% Tween 20 (TBS-T) with 2% BSA (FisherScientific, catalog #BP9704-100). Sections were washed three times in TBS-T w/ 2% BSA and incubated with Alexaflor-568 goat anti-rat secondary antibodies (ThermoFisher, catalog #A11077). After washing sections three times with TBS-T, 40 μl of mountant with DAPI (ThermoFisher, catalog #P36962) and a cover slip were added to each slide. Slides were incubated at room temperature for 24 hours until the mountant solidified.

### Flow Cytometry Analysis of Bacterial Invasion in Tumors

Flow cytometry was used to identify cells in tumors that were invaded by Salmonella and the effect of inducing flhDC on invasion. The types of cells invaded by Salmonella was determined by isolating cells that contained invaded Salmonella and stratifying them into carcinoma, immune and other tumor-associated cells using EPCAM and anti-CD45 antibodies. The effect of inducing flhDC on cell invasion was determined by comparing mice administered flhDC-uninduced and flhDC-induced bacteria and counting the percentage of cells of the three cell types.

Two groups of mice were injected with 2×10⁶ CFU of flhDC Salmonella (Table S1) via the tail vein. To induce production from the PBAD-flhDC gene construct in the flhDC-induced group (n=9), 100 μg of arabinose in 400 μl PBS was administered by intraperitoneal (IP) injection at 48 and 72 hours after bacterial injection. The control, flhDC-uninduced group (n=8) received IP injections at the same times. Ninety-six hours after bacterial injection, mice were sacrificed, and tumors were excised and cut in half. Tumors were processed into single cell suspensions, stained, and analyzed by flow cytometry.

To create a single cell suspension from excised tumors, they were minced with a sterile razor blade in 5 ml of RPMI with 20 mM HEPES, 10% FBS, 1 mg/ml collagenase D (Roche, catalog #11088866001), 200 units/ml of DNAse I (Roche, catalog #04716728001), and 50 μg/ml of gentamicin (ThermoFisher, catalog #BP918-1) to prevent bacterial overgrowth/invasion. Once tumor pieces were less than 5 mm long, the tumor slurry was added to a 7 ml douncer and dounced ten times. The slurry was placed in a single well of a six well plate and incubated at 37° C. for two hours. To separate the cells, the suspension was filtered through a 40 μm cell strainer (ThermoFisher, catalog #22-363-547) and centrifuged for five minutes at 300×g. Red blood cells (RBCs) were lysed by incubating the single cell suspension with RBC lysis buffer (150 mM ammonium chloride, 12 mM sodium bicarbonate and 0.1 mM EDTA) for ten minutes. The cell suspensions were added to 10 ml of D-PBS (Hyclone, catalog #SH30256001) and spun at 300×g for 5 minutes.

Single cell suspensions were fixed in PBS containing 1 mM EDTA and 5% formaldehyde for ten minutes at room temperature. Fixed cells were spun at 600×g for five minutes and resuspended in blocking buffer for one hour. Blocking buffer is TBS-T with 2% BSA and 1 mM EDTA. The 0.1% Tween 20 permeabilizes the cancer cells but not the bacteria as described in the Immunocytochemistry section above. Cell suspensions were sequentially stained with FITC-conjugated anti-Salmonella antibody (Abcam, catalog #ab69253), PE dazzle 594 anti-CD326 (EpCAM; BioLegend, catalog #118236), and APC anti-CD45 (Biolegend, catalog #103112) at concentrations of 1:2000, 1:2000 and 1:1000, respectively. First, anti-Salmonella antibodies were added to cells for 45 minutes, followed by four washed six times with staining buffer (2% BSA, 1 mM EDTA and 0.1% Tween in PBS). Then EpCAM and anti-CD45 were added for 45 minutes, followed by two washes. Fluorescence minus one (FMO) of each sample were used as gating controls for each fluorophore. Samples were analyzed on a custom-built flow cytometer (dual LSRFortessa 5-laser, BD). All fluorophores were compensated with compensation beads (BD, catalog #552845) and did not carry more than 2% bleed over into any other channel. Cells were first identified if they contained intracellular Salmonella. Non-immune cells (cancer and other associated cells) were identified by samples stained with all antibodies except CD45 (i.e. FMO gating controls). Non-cancer cells (immune and other associated cells) were identified by samples stained with all antibodies except anti-EpCAM (CD326).

Effect of flhDC Induction on Bacterial Invasion into Cells in Culture

To determine the effect of expressing flhDC in bacterial invasion, 4T1 cells were grown on glass cover slips as described in the Infection assay section above. Inducible flhDC Salmonella (Table S1) were grown in LB with 20 mM arabinose to induce flhDC expression. Control (flhDC−) bacteria were grown without arabinose. Cancer cells were infected with both induced flhDC+ and flhDC− Salmonella at an MOI of 10 (n=4 for each condition). For the induced flhDC+condition, 20 mM arabinose was added to the mammalian culture to maintain expression. Eighteen hours after invasion, the cancer cells were stained to identify intracellular Salmonella (Salmonella alone, combination 1) as described in the Immunocytochemistry section above. Three images were acquired at 20× for each coverslip, for a total of 12 images per condition. Invasion was quantified by randomly identifying 20 cancer cells from the DAPI channel of each image. Each cell defined as invaded if Salmonella staining was co-localized with the nucleus or was within 10 μm of the nucleus. Invasion fraction was defined as the number of invaded cells over the total number of cells.

Effect of flhDC on Invasion into Tumor Masses In Vitro

To quantify invasion into tumor masses, engineered Salmonella were administered to tumor-on-a-chip devices developed in our laboratory (6, 7). Microfluidic tumor-on-a-chip devices were fabricated using negative tone photoresist and PDMS based soft lithography. Master chips were constructed by spin coating a layer of SU-8 2050 onto a silicon wafer at 1250 RPM for 1 minute. This speed corresponded to an SU-8 2050 thickness of 150 μm. The silicon wafer was baked at 65° C. for 5 minutes followed by 95° C. for 30 minutes. Microfluidic designs printed on a high-resolution transparency were placed over the silicon wafer in a mask aligner. The silicon wafer with the overlaid mask was exposed to UV light (22 J/cm²) for 22 seconds. Silicon wafers were baked for 5 minutes at 65° C. followed by 95° C. for 12 minutes. Wafers were then developed in PGMEA developing solution for 10 minutes and/or until microfluidic features were microscopically distinct with sharp and defined edges.

Soft lithography was used to create the multilayer tumor on a chip device with 12 tumor chambers (two conditions with six chambers each). PDMS (Sylgard 184) at ratios of 9:1 and 15:1 were used for the channel and valve layers, respectively. The channel layer was placed on a spin coater for 1 minute at 220 rpm in order to achieve a PDMS thickness of 200 μm. The silicon wafers were degassed for 45 minutes to eliminate air bubbles in the PDMS. The silicon wafers were baked at 65 degrees for approximately one hour or until both PDMS layers were partially cured. The top valve layer of PDMS was cut and removed from the silicon wafer and aligned on top of the channel layer using a stereomicroscope. The combined layers were baked for one hour at 95° C. in order to covalently bind the two layers. The multilayered PDMS device and a glass slide was plasma treated in a plasma cleaner (Harrick) for 2.5 minutes. Valves were pneumatically actuated with a vacuum pump and the PDMS was placed on the plasma treated glass slide. Valves were actuated until the device was ready for use.

The tumor-on-a-chip was sterilized with 10% bleach followed by 70% ethanol, each for one hour. Microfluidic chips were equilibrated with media (DMEM with 20 mM HEPES, pH 7.4) for one hour. Valve actuation was used to position tumor spheroids in the tumor chambers. Valves at the rear of the chambers were opened while the efflux channel was closed. After the tumor masses were positioned, the valves were reset so that the rear valves were closed and the influx and efflux channels were open.

Prior to administration to the device, flhDC reporting Salmonella (Table S1) were grown in LB with 20 mM arabinose to induce flhDC expression. These Salmonella have inducible flhDC (PBAD-flhDC) and produce GFP when intracellular (PsseJ-GFP). Control (flhDC−) Salmonella of the same strain were grown without arabinose. The bacteria were centrifuged and resuspended in culture medium (DMEM with 20 mM HEPES) at a density of 2×10⁷ CFU/ml. For the induced flhDC+ condition, 20 mM arabinose was added to the medium. Bacteria-containing media (flhDC+ and flhDC−; n=6 chambers each) were perfused through the tumor-on-a-chip devices for one hour at 3 μm/min for a total delivery of 2×10⁶ CFU to each device. Bacterial administration was followed by bacteria-free media (with 20 mM HEPES) for 48 hours.

Devices were imaged at 30-minute intervals. Invasion was quantified at 31 h by measuring GFP expression by invaded bacteria in the tumor masses. Regions of interest were defined around the borders of the tumor masses. The extent of invasion was determined as the average GFP fluorescence intensity in each tumor mass. Intensities were normalized by the intensity of the average tumor mass administered control (flhDC−) Salmonella.

### Intracellular Activation of the PsifA and PsseJ Promoters

Salmonella with GFP-reporting constructs for the PsifA and PsseJ promoters were grown in LB. These Intracellular reporting and PsifA strains contain constructs PsseJ-GFP and PsifA-GFP, respectively (Table S1). Both bacterial strains were administered to MCF7 cancer cells in six well plates at an MOI of 25 as described in the Invasion Assay section above. Live cells were imaged at 20× magnification, three hours after invasion. Images of extracellular bacteria were acquired in LB culture in six well plates at 20×. Extracellular promoter activity was determined as the average fluorescence intensity of bacteria from three wells each and normalized to the average intensity of PsseJ bacteria. The increase in promoter activity following cellular invasion was determined by averaging the fluorescence intensity of bacteria in cells in three wells and comparing it to the average intensity of extracellular bacteria.

### Bacterial Death Caused by Inducing Expression of Lysin E

Salmonella strain PBAD-LysE (Table S1) was grown in LB in 3 ml culture tubes to an average OD of 0.25. OD was measured every 30 minutes for three hours. After 90 minutes of growth, three of the cultures were induced with 10 mM arabinose. Arabinose was not added to three control cultures. Growth and death rates were determined by fitting exponential functions to bacterial density starting at time zero (for growth) and 90 minutes (for bacterial death).

### Intracellular Lysis and GFP Delivery

To visualize and quantify triggered intracellular lysis and GFP delivery, ID Salmonella were administered to cancer cells on coverslips and in well plates as described in the Invasion Assay section above. ID Salmonella constitutively express GFP (Plac-GFP) and express Lysin E after activation of PsseJ (PsseJ-LysE).

To quantify the extent and rate of lysis, ID Salmonella were administered to MCF7 cancer cells at an MOI of 25. Parental Salmonella that constitutively express GFP (transformed with plasmid pLacGFP) were used as controls. Transmitted-light images of cancer cells and fluorescent images of bacteria were acquired at 20× every 30 minutes for 10 hours. From three wells, 200 cancer cells were randomly selected from the first transmitted image for each condition. Over the time of the experiment, cells were scored if any bacteria invaded and when these intracellular bacteria lysed. The lysis fraction was defined as the number of cells with lysed bacteria over the total number of observed cells. The rate of intracellular lysis was determined by binning the number of cells with lysed bacteria per hour and fitting an exponential function to the cumulative fraction of cells with lysed bacteria.

The comparison of growth and death rates were (1) the growth rate of parental Salmonella in LB, (2) the growth rate of PBAD-LysE Salmonella in LB, (3) the death rate of PBAD-LysE Salmonella after induction with arabinose, (4) the growth rate of Pssei-LysE Salmonella in LB, and (5) the lysis (death) rate of Pssei-LysE Salmonella after invasion into cancer cells.

To generate images of bacterial lysis and GFP delivery, ID Salmonella were administered to 4T1 cancer cells grown on coverslips at an MOI of 10. After six hours, the coverslips were fixed and stained for Salmonella and released GFP (antibody combination #2) as described in the Immunocytochemistry section above. Images were acquired at 100× with oil immersion.

### Bacterial Protein Content

To quantify the amount of produced GFP, ID Salmonella (Table S1) were grown in LB. The bacteria were centrifuged, washed and resuspended at four densities: 10⁶, 10⁷, 10⁸, and 10⁹ bacteria per 40 μl Laemmli buffer, which lysed the bacteria. A GFP standard was loaded at three concentrations: 1, 10 and 100 ng per 40 μl Laemmli buffer. Samples were boiled and loaded onto NuPAGE 4-12% protein gels (Invitrogen, catalog #NPO0321BOX) in MOPS buffer. Resolved gels were transferred to PVDF blotting paper. Membranes were blocked with 2% bovine serum albumin in Tris-buffered saline with 5% skim milk powder and 0.1% Tween 20 (TBST+milk) for 1 hour. Blots were incubated with rat anti-GFP monoclonal antibody (Chromotek, catalog #3h9-100) primary antibody in TBST+milk overnight. Blots were washed three times with (TBST) and incubated with HRP-conjugated goat anti-rat secondary antibody (Dako, catalog #X0909) for one hour at room temperature in TBST-milk.

### Lysis and GFP Release in Cells and SCVs

In order to assess GFP release from vacuoles, ID Salmonella where administered to 4T1 cancer cells. A specialized staining technique was used to identify SCVs and isolate released GFP from un-released, intra-bacterial GFP. The 4T1 cells were grown on glass coverslips were infected with ID Salmonella (Table S1) at an MOI Of 10 using the methods described in the Invasion Assay section.

At two time points, 6 and 24 hours, four coverslips were fixed and permeabilized as described in the Immunocytochemistry section above. The blocking buffer used for permeabilizing the cells contained Tween 20, which selectively permeabilized mammalian, but not bacterial cell membranes. This allowed primary antibodies to bind GFP in the mammalian cytoplasm, but not inside un-lysed bacteria. After permeabilization, cells were stained for Salmonella, released GFP, and vacuoles (combination 3) in the Immunocytochemistry section) using anti-Salmonella, anti-myc, and anti-LAMP1 antibodies.

After mounting, coverslips were imaged under oil immersion at 100× magnification. Acquired images were background subtracted and borders were drawn around cells (n=24 at 6 h, and n=7 at 24 h). Released GFP was divided into two groups: vacuolar and cytosolic. Vacuolar GFP was surrounded by LAMP1-stained regions. Cytosolic GFP was all other GFP inside cells. For each cell, the vacuolar and cytosolic GFP fractions were determined as the sum of pixel intensities in the region divided by the sum of intensities in both regions (i.e. the total in the cell). To visualize the localization of released GFP in cells over time, ID Salmonella were administered to 4T1 cancer cells. The cancer cells were grown on glass coverslips were infected with ID Salmonella (Table S1) at an MOI Of 10. At two time points, 6 and 24 hours, four coverslips were fixed and permeabilized as described above. The cells were stained for Salmonella, released GFP, and β-actin (combination 2) with anti-Salmonella and anti-myc antibodies, and phalloidin. Actin staining enables visualization of structures and boundaries. Images were acquired at 100× with oil immersion.

### Dynamic Measurement of GFP Release and Diffusion

To measure the rate of GFP dispersion through cells after lysis, MCF7 cancer cells were grown on 96-well plates with coverslip glass bottoms for imaging (ThermoFisher, catalog #160376). ID Salmonella were administered at an MOI Of 25 using the methods for live-cell imaging as described in the Invasion Assay section. After washing away extracellular bacteria and adding gentamycin, one cell with intracellular bacteria was identified, and transmitted and fluorescence images were acquired at 63× every minute for 14 hours. This process was repeated ten times. Fluorescence images were selected to start with intact bacteria and end after GFP diffusion. These images were converted into stacks in Zen (Zeiss) and intensities were measured on lines passing through bacterial centers at time zero (before lysis) until diffusion was complete. The GFP spatiotemporal intensity profiles were fit to the radial diffusion equation.

$$\frac{\partial C}{\partial t} = 𝒟 \frac{1}{{r}^{2}} \frac{\partial }{\partial r} ( {r}^{2} \frac{\partial C}{\partial r} ) \qquad ( 1 )$$

In this equation, C is the GFP concentration and D is the effective diffusivity of GFP in the cytosol. When there is an instantaneous release of material at t=0 from r=0 (i.e. lysis), equation (1) has an analytical solution.

$$C ( r , t ) = {( 4 π𝒟 t )}^{\frac{2}{3}} exp ( \frac{- {r}^{2}}{4 𝒟 t} ) \qquad ( 2 )$$

Cytosolic diffusivity of released GFP, D, was determined be fitting the GFP intensity profiles to equation (2) using least-squared fitting.

### Location of GFP Release

To quantify the location of GFP release in cells, ID Salmonella where administered to 4T1 cancer cells on glass coverslips at an MOI Of 10 using the methods in the Invasion Assay section. At 6 hours, three coverslips were fixed, permeabilized and stained to identify Salmonella, released GFP, and vacuoles (combination 3) in the Immunocytochemistry section) using anti-Salmonella, anti-myc, and anti-LAMP1 antibodies. After mounting, coverslips were imaged under oil immersion at 100× magnification. Acquired images were background subtracted and Salmonella were identified in seven 86.7×66.0 μm regions across the three coverslips. Every bacterium within the regions was classified as un-lysed or lysed if co-localized with released GFP. The location of each lysed Salmonella was determined based on co-localization with LAMP1 staining as inside or outside SCVs. The fraction of released GFP in vacuoles was the number of lysed Salmonella in SCVs over total lysed Salmonella.

### Dependence of Protein Release on Residence in SCVs

To determine the dependence of protein release on residence in SCVs, ID Salmonella with two gene knockouts were administered to cancer cells. 4T1 cancer cells were grown on coverslips and infected with ΔsifA, ΔsseJ, or ID Salmonella (n=3 for each condition). All three of these strains contained the PsseJ-lysE and Plac-GFP-myc gene circuits (Table S1). The ΔsifA strain predominantly accumulates in the cellular cytoplasm and the ΔsseJ strain predominantly accumulates in SCVs and does not escape into the cytoplasm. Bacteria were administered at an MOI of 10 as described in the Invasion Assay section. At 6 hours after invasion, the cancer cells were fixed, permeabilized and stained for Salmonella and released GFP as described in the Immunocytochemistry section. Nine images from three coverslips were acquired at 20× for each condition. Images were background subtracted. Lysis fraction was calculated using pixel by pixel image analysis in MATLAB. Lysis was identified as pixels that positively stained for GFP-myc. The permeabilization technique prevented staining of GFP inside un-lysed Salmonella. Un-lysed Salmonella were identified as pixels that stained for Salmonella but not GFP-myc. Total bacterial pixels is the sum of these values. Lysis fraction is the number of lysis pixels over total bacterial pixels.

### Dependence of Protein Delivery on Invasion and Intracellular Lysis

Four strains of Salmonella were administered to cancer cells to determine the necessity of the two engineered gene circuits, PsseJ-LysE and PBAD-flhDC, on protein delivery. Two strains were used: flhDC Sal and flhDC-ID Sal (Table S1). Both of these strains have flhD deleted and only express flhDC after induction with arabinose. The flhDC-ID Sal strain also contains the PsseJ-LysE circuit which induces lysis after cell invasion. Prior to invasion, two cultures of flhDC Sal and flhDC-ID Sal bacteria were grown in LB with 20 mM arabinose to induce flhDC expression. Two cultures were grown without arabinose. For microscopy analysis, 4T1 cancer cells were grown on coverslips and infected at an MOI of 10 with one of the four strains: PsseJ-LysE−, flhDC−; PsseJ-LysE−, flhDC+; PsseJ-LysE+, flhDC−; or PsseJ-LysE+, flhDC+. For flow cytometry, 4T1 cells were grown on six well plates and infected at an MOI of 10 with the same four strains. On both coverslips and well plates, 20 mM arabinose was added to the two induced flhDC+ conditions to maintain expression.

For microscopy, coverslips were fixed, permeabilized and stained for released GFP as described in the Immunocytochemistry section. Nine images for each condition were acquired at 20× magnification and background subtracted. Protein (GFP) delivery was determined using pixel by pixel image analysis in MATLAB. A pixel was positive for delivery if it stained for GFP-myc. Total delivery was calculated as the sum of the intensities of all delivery positive pixels. Values were normalized by the PsseJ-LysE−, flhDC− condition.

For flow cytometry, cells were processed into a single cell suspension by gently pipetting after washing with PBS and adding 0.05% trypsin (ThermoFisher, catalog #25300-054). Cells were fixed with 5% formaldehyde in PBS w/ 1 mM EDTA and incubated in blocking buffer for 30 minutes. Cells were intracellularly stained with a 1:2000 dilution of FITC-conjugated anti-Salmonella antibody (Abcam, catalog #ab69253), and a 1:200 dilution of rat anti-myc monoclonal antibody (Chromotek, catalog #9e1-100) for 30 minutes. Cells were washed three times with blocking buffer. Cells were incubated with DyLight 750 anti-rat secondary antibody (ThermoFisher, catalog #SA5-10031) at a 1:200 dilution for one hour at room temperature. Samples were analyzed on a custom-built flow cytometer (dual LSRFortessa 5-laser, BD). All fluorophores were compensated with compensation beads (BD, catalog #552845) and did not carry more than 2% bleed over into any other channel. Control cells that were not infected by Salmonella were used as gating controls to identify uninfected cells in the samples, based on Salmonella staining. Cells administered non-lysing bacteria (i.e., PsseJ-LysE−) were stained with anti-Salmonella antibody, anti-rat secondary antibody, but not the anti-myc primary antibody to identify cells without GFP delivery.

Intracellular Delivery of GFP to Cells in Tumors with ID Salmonella

To identify and quantify GFP delivery to tumor cells, five BALB/c mice with 4T1 tumors were injected with 2×10⁶ CFU of ID Salmonella (Table S1). Ninety-six hours after bacterial injection, mice were sacrificed and tumors, liver and spleens were excised. Tumors were cut in half. One half was fixed and stained for imaging and the other half was cryopreserved for protein quantification. Livers and spleens were also cryopreserved. Fixed tumors were embedded, sectioned and deparaffinized as described in the Immunohistochemistry section. Tumor sections were stained to identify GFP with a 1:50 dilution of goat anti-GFP (Abcam, ab6556) overnight, followed by incubation with a 1:50 dilution of Alexa Fluor 488-conjugated donkey anti-goat antibody (ThermoFisher, catalog #A21208) at room temperature for 1 h. After counterstaining with DAPI and mounting, sections were imaged at 20×.

To quantify the amount of delivered protein, half of the tumors as well as the livers and spleens were snap-frozen in liquid nitrogen and stored at −80° C. Lysates were made in a buffer containing 50 mM Tris-HCl at pH 7.4, 0.3% Triton-X 100, 0.1% NP-40 and 0.3 M NaCl. The buffer was supplemented with 25 mM NaF, 5 μM leupeptin, 0.5 mM phenylmethanesulfonyl fluoride, 0.5 mM benzamidine and 1 mM dithiothreitol. As with cancer cells in culture, this buffer lyses mammalian cells but not bacterial membranes, thereby separating delivered protein from protein in intact bacteria. Samples were homogenized on ice using a blender (Polytron) and a homogenizer (Potter-Elvehjem). Samples were incubated for 20 minutes on ice, centrifuged for 10 minutes at 664×g and 4° C. and the supernatant was collected. Immunoblotting was performed following 10% SDS-PAGE with anti-GPF (Abcam, catalog #ab6673) and anti-β-actin (GeneTex, catalog #GTX26276, clone AC-15). Immunoblots were visualized using eCL reagent (PerkinElmer) on a ImageQuant LAS4000 imaging system (GE Healthcare).

Effect of flhDC on Protein Delivery in Mice

To determine the effect of flhDC on protein delivery, nine BALB/c mice with 4T1 tumors were injected with 2×10⁶ CFU of flhDC-ID Salmonella (Table S1) via the tail vein. Prior to injections, cultures of flhDC-ID Sal were grown in LB with 20 mM arabinose to induce flhDC expression. A second culture was grown without arabinose. At 48 and 72 hours after bacterial injection, 100 μg of arabinose in 400 μl of PBS was injected intraperitoneally into the flhDC+mice to maintain expression. The flhDC− mice received intraperitoneal injections of PBS at the same times. Ninety-six hours after bacterial injection, mice were sacrificed and tumors (n=4 for flhDC− and n=5 for flhDC+) were excised and sectioned as described in the Immunohistochemistry section. Tumor sections were stained to identify GFP with rat anti-GFP monoclonal antibody (Chromotek, catalog #3h9-100) and Alexaflor-568 goat anti-rat secondary antibodies (ThermoFisher, catalog #A11077). After counterstaining with DAPI, sections were imaged at 10× magnification. Images were background subtracted and were analyzed with computational code in MATLAB. Delivery was quantified at 20 random points in the transition zones of each tumor. A point was scored as positive if a cell within 20 μm contained delivered GFP. A cell was considered to have delivered protein if the GFP filled the entire cytoplasm. The delivery fraction is the number of positive points divided by the total number of random points.

### Temporal Colonization of ID Salmonella in Tumors

To determine tumor density over time, 2×10⁷ CFU ID Salmonella that express luciferase (ID Sal-luc, Table S1) were intravenously injected into five BALB/c mice with orthotopic 4T1 tumors in the mammary fat pad. Bacterial colonization was followed in real time by bioluminescent imaging. At 24, 48, 72, 168, 336 hours after bacterial injection, mice were injected i.p. with 100 μl of 30 mg/ml luciferin in sterile PBS, anesthetized with isoflurane, and imaged with an IVIS animal imager (PerkinElmer, SpectrumCT). Bacterial density in tumors was determined as the proton flux from the tumors. After acquiring the final image (at 14 days), tumors were excised and minced in equal volumes of sterile PBS. Homogenized tumors were cultured on agar plates. Colonies were counted after overnight growth at 37° C.

### Biodistribution and Toxicity of ID Salmonella

To determine the biodistribution of Salmonella, five tumor-free BALB/c mice were injected with 1×10⁷ ID Salmonella. After 14 days, six organs were excised and weighed: spleen, liver, lung, kidney, heart and brain. Organs were minced in equal volumes of sterile PBS, diluted 10 and 100 times, and cultured on agar plates. Colonies were counted after overnight growth at 37° C. To measure the toxicity of ID Salmonella, four tumor-free BALB/c mice were injected with 1×10⁷ ID Salmonella. Four control mice were injected with sterile saline. After 14 days, whole blood was isolated from anesthetized mice by percutaneous cardiac puncture. Collected blood was divided between clot-activating serum tubes and EDTA anticoagulant tubes for chemistry and CBC analyses, respectively. Chemistry profiling and comprehensive hematology was conducted on the serum and whole blood samples by Idexx Laboratories (Grafton, MA).

Delivery of Nanobodies with ID Salmonella

To measure the delivery of nanobodies, ID Salmonella were administered to cancer cells and the extent of binding to the protein target was determined by immunoprecipitation. 4T1 cancer cells were grown to 80% confluency in T75 flasks and infected with either NB or ID Salmonella (as controls; Table S1) at an MOI of 10 as described in the Invasion assay section. The β-actin nanobody expressed by NB Salmonella is tagged with the FLAG sequence at the C terminus. Prior to administration, NB Salmonella were grown in LB with 20 mM arabinose to induce nanobody expression and 20 mM arabinose was added to the NB cultures to maintain expression. Twenty-four hours after invasion, the cancer cells were harvested using a cell lifter and centrifuged at 600×g for 10 minutes. The cell pellet was resuspended in 10 ml of lysis buffer (20 mM HEPES, 1 mM EDTA, 10% glycerol w/v, 300 mM sodium chloride and 0.1% Tween) that only lysed cancer cells but not intact bacteria. The cell suspension was homogenized in a douncer using a tight plunger. The cell lysate was clarified by centrifugation at 20,000×g for 20 minutes at 4° C. The lysate was incubated with 50 μl of anti-FLAG purification resin (Biolegend, catalog #651502) overnight at 4° C. The FLAG resin was washed three times with lysis buffer. Fifty microliters of Laemmli buffer was added directly to the bead solution and boiled for 5 minutes at 95° C. Boiled beads were loaded onto SDS-PAGE gels (15% polyacrylamide, cast in-house) in MOPS buffer for Western blotting as described in the Bacterial protein content section. Gels were transferred to nitrocellulose blotting paper. Blots were incubated with mouse anti-actin monoclonal antibody (Cell Signaling Technology, catalog #8H10D10) and HRP-conjugated goat anti-mouse secondary antibodies (ThermoFisher, catalog #31450) to identified β-actin.

### Cytotoxicity of Delivery of CT-Casp-3 and NIPP1-CD to Cells in Culture

To measure the cytotoxicity of delivering protein drugs, ID Salmonella were administered to cancer cells in culture. Hepa 1-6 liver cancer cells were grown in six well plates to 80% confluency. NIPP1-CD, CT-Casp-3 Salmonella, and control ID Salmonella were administered at MOI of 10 as described in the Invasion assay section. Prior to invasion, cultures of CT-Casp-3 Salmonella were grown in LB with 20 mM arabinose for one hour to induce expression of CT-Casp-3. To all wells, 20 mM arabinose was added to maintain expression. Ethidium homodimer (500 ng/ml) was added to each well to stain dead cells with permeable membranes. Three mages were acquired per well (for nine images per condition) every 30 minutes for 24 hours at 20× magnification. At each time one transmitted and two fluorescent images were acquired: bacterial produced GFP (480/525 excitation/emission) and ethidium homodimer (525/590 excitation/emission). Images were background subtracted. From the fluorescent time-lapse images, cancer cells were identified that were invaded by Salmonella. Cell death was calculated as the fraction of dead Salmonella-invaded cells (co-localized with ethidium homodimer staining) over the total number of Salmonella-invaded cells.

### Delivery of CT-Casp-3 and NIPP1-CD to Tumor Masses

To measure cell death in tumor masses after delivery of CT-Casp-3 or NIPP1-CD, ID Salmonella were administered to tumor-on-a-chip devices. Microfluidic devices were fabricated as described in the Effect of flhDC on invasion into tumor masses in vitro section. Two independent device experiments were run: (1) NIPP1-CD vs. ID control Salmonella with six chambers each; and (2) CT-Casp-3 vs. ID control Salmonella with four and three chambers, respectively. Prior to administration to the device, CT-Casp-3 Salmonella were grown in LB with 20 mM arabinose to induce expression of CT-Casp-3. NIPP1-CD and ID Salmonella were grown in LB without arabinose. All bacteria were centrifuged and resuspended in culture medium (DMEM with 20 mM HEPES) at a density of 2×10⁷ CFU/ml. For CT-Casp-3 Salmonella, 20 mM arabinose was added to the medium. Bacteria-containing media, containing 500 ng/ml ethidium homodimer, was perfused through the tumor-on-a-chip devices for one hour at 3 μm/min for a total delivery of 2×10⁶ CFU to each device. Bacterial administration was followed by bacteria-free media, with 20 mM HEPES and ethidium homodimer. Transmitted and fluorescence images were acquired every 30 minutes for 24 hours at 5× magnification. Death was calculated by first defined the borders of the tumor masses. Florescence images were segmented to identify regions of dead cells that stained with ethidium homodimer. The extent of death was the fraction of the tumor mass that was dead.

### The Final Fraction of Death was Determined at 24 h.

Tumor response to delivery of CT-Casp-3 in mice Two mouse models were used to measure the effect of delivering CT-Casp-3: 4T1 murine breast cancer cells in BALB/c mice and Hepa 1-6 murine liver cancer cells in C57L/J mice. For both models, three conditions were tested by injecting saline, ID Salmonella, or CT-Casp-3 Salmonella. The saline controls establish the baseline growth rate of the tumors. The ID Salmonella (bacterial) control established the effect of colonized bacteria and intracellular lysis on the tumor growth rate. For both mouse models, three groups of six mice were subcutaneously injected with 1×10⁵ tumor cells. Once tumors were between 50 and 75 mm³, they were injected with one of the three conditions: saline or 4×10⁷ CFU of ID or CT-Casp-3 Salmonella. At 48 and 72 hours after injection, mice were injected i.p. with 100 mg of arabinose in 400 μl of PBS. Every five days, tumors were injected with bacteria or saline. Tumors were measured twice a week and volumes were calculated with the formula (length)*(width²)/2. Mice were sacrificed when tumors reached 1000 mm³. Tumor growth rates were determined by fitting exponential functions to tumor volumes as functions of time.

### Statistics

For pair-wise comparisons, Student's t test was used. Statistical significance was confirmed when P<0.05. ANOVA with a Bonferroni correction was used when comparing multiple data points.
1. Swofford, C. A., N. Van Dessel, and N. S. Forbes, Quorum-sensing Salmonella selectively trigger protein expression within tumors. Proceedings of the National Academy of Sciences of the United States of America, 2015. 112(11): p. 3457-62.
2. Mosberg, J. A., M. J. Lajoie, and G. M. Church, Lambda red recombineering in Eschenchia coli occurs through a fully single-stranded intermediate. Genetics, 2010. 186(3): p. 791-9.
3. Vaidya, S., et al., Substrate-induced conformational changes occur in all cleaved forms of caspase-6. J Mol Biol, 2011. 406(1): p. 75-91.
4. Yan, Y., et al., Asd-based balanced-lethal system in attenuated Edwardsiella tarda to express a heterologous antigen for a multivalent bacterial vaccine. Fish Shellfish Immunol, 2013. 34(5): p. 1188-94.
5. Nakayama, K., S. M. Kelly, and R. Curtiss, Construction of an ASD+ expression-cloning vector—stable maintenance and high-level expression of cloned genes in a Salmonella vaccine strain. Bio-Technology, 1988. 6(6): p. 693-697.
6. Toley, B. J. and N. S. Forbes, Motility is critical for effective distribution and accumulation of bacteria in tumor tissue. Integr Biol, 2012. 4(2): p. 165-76.
7. Walsh, C. L., et al., A multipurpose microfluidic device designed to mimic microenvironment gradients and develop targeted cancer therapeutics. Lab on a chip, 2009. 9: p. 545-54.

### Results and Discussion

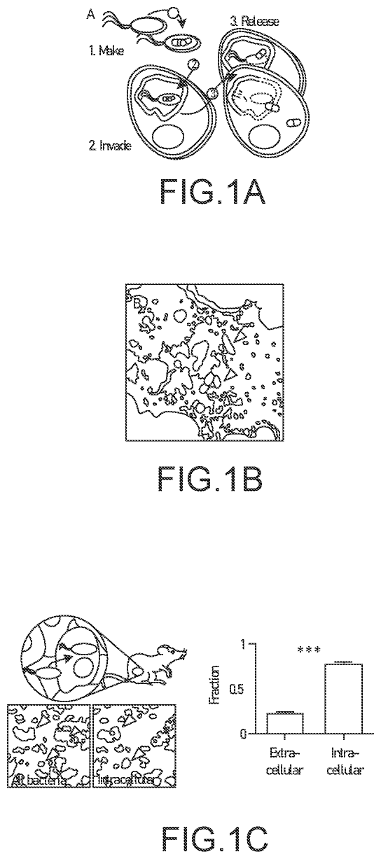

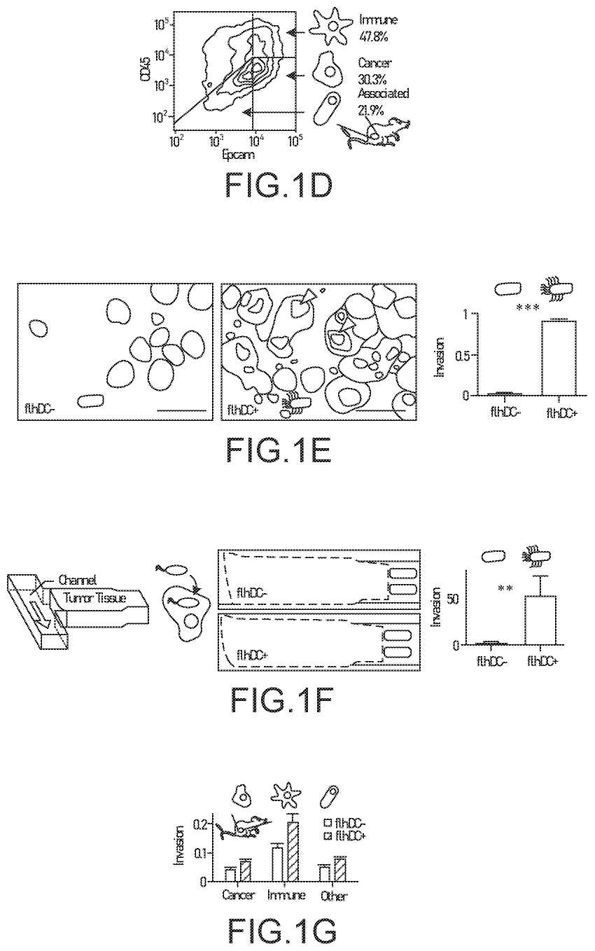

Herein, the creation of an intracellular protein delivery system based on the natural qualities of Salmonella is described (FIG. 1A). In the intestines, Salmonella have a partially intracellular lifestyle. To evade clearance, Salmonella invade epithelial cells using the proteins expressed by Salmonella pathogenicity island 1 (SPI1) (3,4). After invasion, Salmonella reside in early and late endosomes, which they reform into Salmonella containing vacuoles (SCVs) by expressing the genes of pathogenicity island 2 (SPI2) (5-7). SCVs enable intracellular survival (5,8) and protect the Salmonella from intracellular defense mechanisms (9,10). A step in the activation of SPI2 genes, is the sensing of the endosomal environment. These sensing mechanisms, which are unique to Salmonella, are needed for delivery of proteins into cells.

While it is well established that Salmonella invade intestinal cells (4,11), their location within tumors is more uncertain, despite extensive documentation of tumor colonization (12-16). Preferential accumulation and exponential growth in tumors are essential properties of therapeutic Salmonella (17,18). When administered in culture, Salmonella readily invade into carcinoma cells (FIG. 1B). To determine where they reside in tumors, Salmonella with a fluorescent intracellular reporter were injected into tumor-bearing BALB/c mice. In these tumors, over 70% of Salmonella were intracellular (P<0.001, n=5, FIG. 1C), demonstrating their suitability as delivery vehicles. In cells dissociated from tumors with collagenase, bacteria were present in carcinoma, immune, and other tumor associated cells (FIG. 1D).

The development of therapeutic Salmonella into an intracellular protein delivery system had three steps (FIG. 1A). The design goals were to engineer Salmonella to (1) Make a drug, (2) Invade into cells, and (3) Release the drug into cells. The use of bacteria changes what is traditionally meant by “delivery.” Unlike typical delivery vehicles, bacteria manufacture protein drugs at the disease site (19), delivering exponentially more molecules than were originally present in the injected bacteria. Chronologically, the first steps were to generate a platform strain with controlled invasion and release. The last step was to transform this platform strain with genes to synthesize different protein drugs. In the final engineered strains of Intracellular Delivering (ID) Salmonella, each of these three processes (make, invade and release) was controlled by a specialized genetic circuit.

In this system, invasion of ID Salmonella into cells is controlled with the regulation factor flhDC(FIG. 1E-G). Expression of flhDC is required for Salmonella to invade cancer cells (FIG. 1E). When flhDC is not expressed, Salmonella invaded less than 2% of cells, which was 54 times less than by Salmonella with re-expressed flhDC (84%; P<0.001; FIG. 1E). Invasion is dependent on flhDC because it regulates the production of flagella and the type III secretion system (20). In microfluidic tumor masses in vitro (21), re-expression of flhDC increased cell invasion and colonization 53 times (P<0.01, FIG. 1F). In tumors, re-expression of flhDC increased invasion into both carcinoma and immune cells (P<0.05, FIG. 1G).

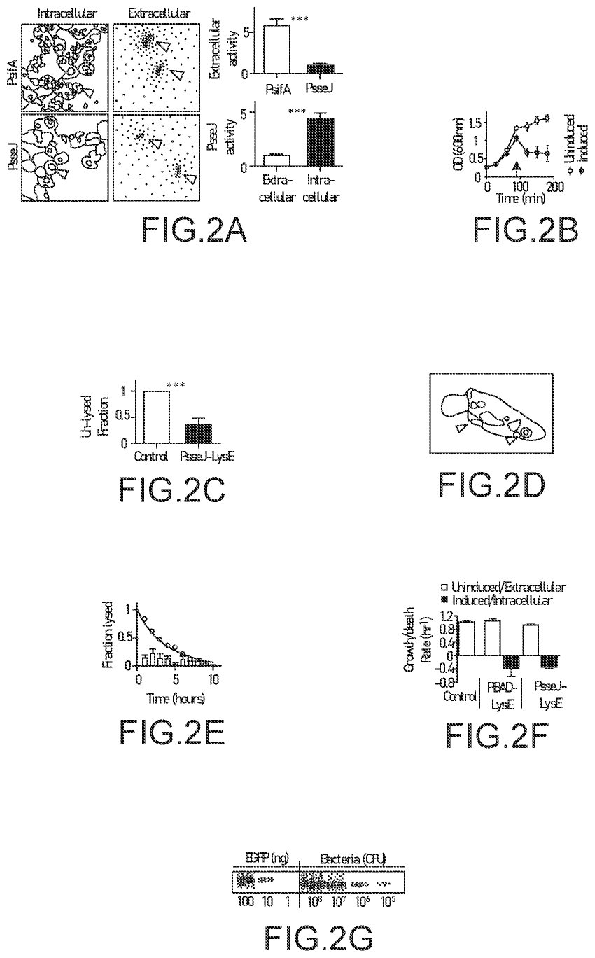

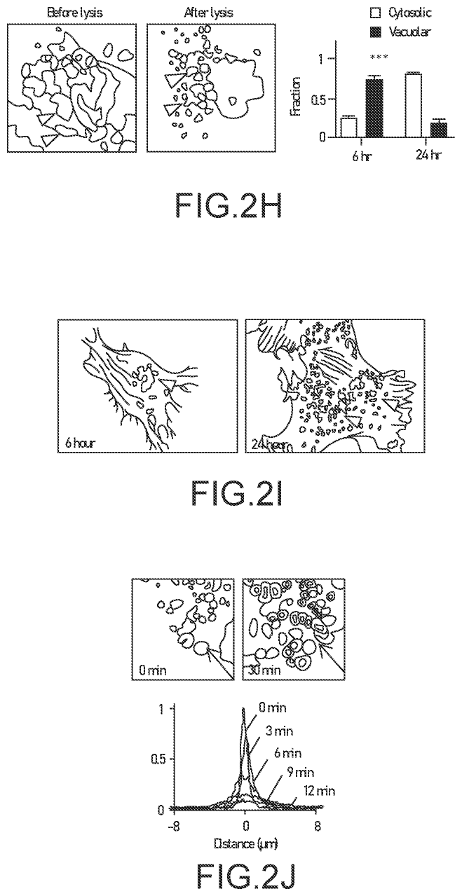

The second component of ID Salmonella, release, required development of a system to trigger autonomous lysis after cell invasion (FIG. 2). This goal was achieved by identifying a Salmonella promoter that is triggered intracellularly and not extracellularly. After invasion into cells, the genes of SPI2 activate to form Salmonella containing vacuoles (SCVs) (8). When coupled to a GFP reporter, the promoters of two SPI2-associated genes, PsseJ and PsifA, both activate after invasion into cancer cells (FIG. 2A, left). However, the extracellular expression of PsseJ is 5.8 times less than PsifA (P<0.001, FIG. 2A), indicating that it is more sensitive to cell invasion.

To release a synthesized protein cargo, the bacteria must lyse after invasion. Triggered expression of Lysin gene E (LysE) from bacteriophage DX1174 causes rapid bacterial death (FIG. 2B). Salmonella, with the coupled PsseJ-LysE construct and that constitutively expresses GFP (as a model protein drug), lysed after invasion into cancer cells (FIG. 2C), and discharged GFP into the cytoplasm (FIG. 2D). Bacterial lysis occurs for 10 hours after invasion (FIG. 2E). The basal expression of Lysin E by the PsseJ-LysE circuit does not affect bacterial health and intracellular induction activated the system at near to its maximum rate (FIG. 2F). Each bacterium can deliver, on average, 163,000 GFP molecules (FIG. 2G).

After bacterial lysis, delivered protein escapes SCVs and fills the cellular cytoplasm (FIG. 2H-I). This escape is important because, immediately after invasion, most Salmonella reside within SCVs (FIG. 2H, left). When ID Salmonella lyse, clusters of released GFP protein are contained within SCVs (FIG. 2H, middle). Over time, the protein escapes the SCVs and fills the entire cytoplasm (FIG. 2I), a transition that occurs for most cells (P<0.001, FIG. 2H, right). GFP diffuses through the cytoplasm with an effective diffusivity of 0.15 μm2/min (FIG. 2J).

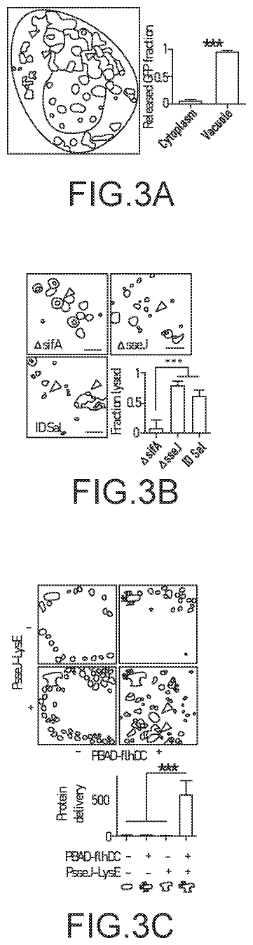

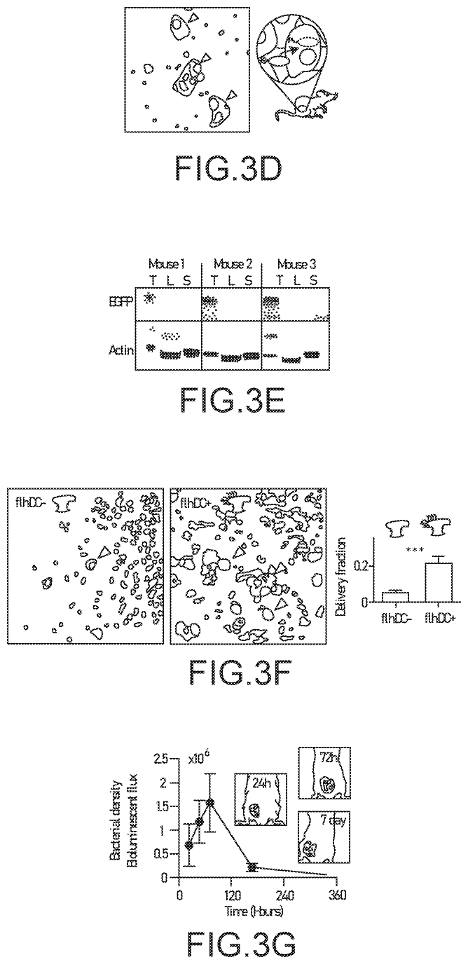

As designed, bacterial lysis is dependent on residence within SCVs (FIG. 3A-B). After invasion, some ID Salmonella escape into the cytoplasm and are not surrounded by a SCV membrane (FIG. 3A, left). More than 95% of GFP released from Salmonella originated inside SCVs (P<0.001; FIG. 3A, right). After invasion into cancer cells, ID Salmonella with a ΔsifA deletion, which are predominantly cytoplasmic (23), did not lyse despite containing the PsseJ-LysE construct. Comparatively, ID Salmonella with a ΔsseJ, which are predominantly vacuolar (24), almost all lysed (P<0.001, FIG. 3B). Without these deletions, most ID Salmonella localized to SCVs, lysed and delivered protein (P<0.001, FIG. 3B). This dependence indicates that the Pssej promoter only activates after SCV localization and not when in the cytoplasm. This specific sensing of the SCV environment is a feature exclusive to Salmonella.

Protein delivery was dependent on the two engineered systems, PBAD-flhDC for invasion and PsseJ-LysE for release (FIG. 3C). Salmonella without flhDC expression did not invade cells, and Salmonella without Pssei-LysE did not release the GFP cargo (FIGS. 3C&S2). Compared to controls, the presence of both systems increased protein delivery 548 times (P<0.001; FIG. 3C).

When administered systemically to tumor-bearing mice, ID Salmonella specifically deliver protein to tumor cells, and this delivery is dependent on flhDC (FIG. 3D-F). ID Salmonella invaded cells and delivered GFP that filled the cellular cytoplasm (FIG. 3D). This system delivered 60 t 12 μg GFP/g tumor (FIG. 3E), which is equivalent to 1.5×10⁸ bacteria per gram of tumor. No GFP was detected in the livers or spleens of any mice (FIG. 3E). When tumor-bearing mice were administered ID Salmonella that did not express flhDC, little GFP was delivered (FIG. 3F). Re-expressing flhDC increased the percentage of cells that received GFP more than five times (P<0.001).

Delivery of proteins with ID Salmonella is safe and self-limiting (FIG. 3G). After intravenous administration, the tumor density of ID Salmonella reached a peak at 72 h and then dropped 97% in 11 days (FIG. 3E). The decline in density, which was caused by intracellular lysis, limits the exposure to therapy and increases safety compared to non-lysing Salmonella. After administration to healthy, tumor-free mice, ID Salmonella did not accumulate in lungs, hearts, kidneys or brains; had no effect on liver function; and caused no adverse immune responses.

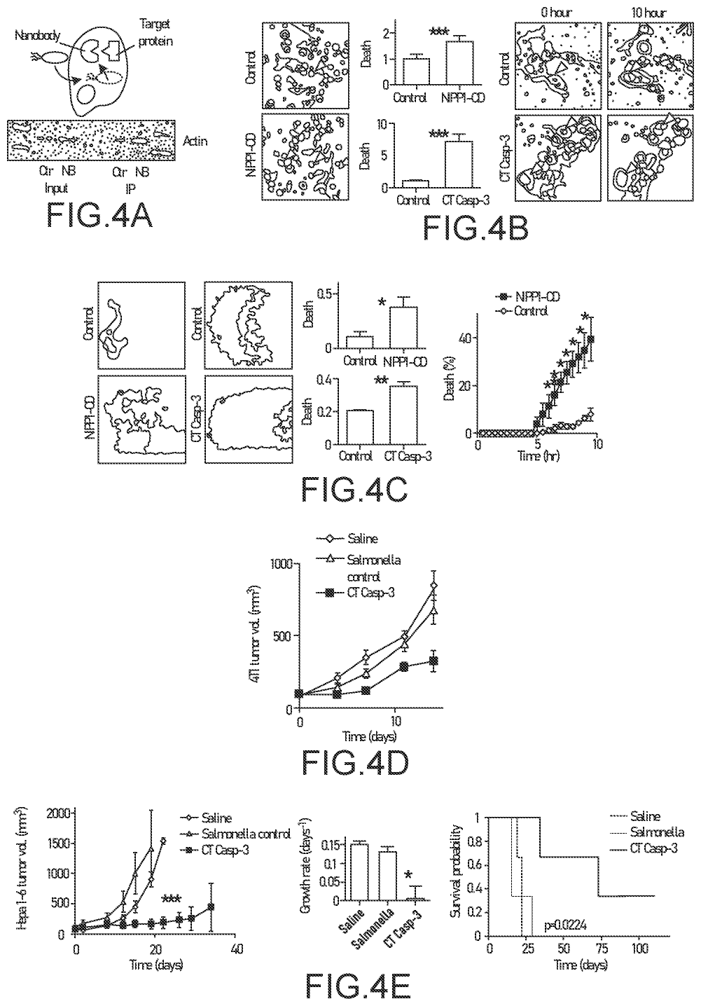

To demonstrate its broad capabilities, ID Salmonella was engineered to make three different proteins (FIG. 4) that affect intracellular physiology: a nanobody (anti-actin), a protein inhibitor (NIPP1-CD), and an endogenous protein (CT casp-3). The central domain of nuclear inhibitor of protein phosphatase 1 (NIPP1-CD) removes PP1 from its holoenzymes and induces cell death (25). Constitutive two-chain active caspase-3 (CT Casp-3) is an engineered active form of caspase-3, the dominant executioner caspase that leads to apoptotic cell death (26, 27).

In one aspect, a bicistronic mRNA codes for caspase, with, for example, the large subunit followed by a ribosomal binding site and the small subunit on, for example, PBAD inducible promoter.

active caspase 3 sequence (bicistronic mRNA-FLAG-large subunit,
RBS, small subunit-myc)
(SEQ ID NO: 200)
ATGGACTACAAAGACGATGACGACAAGATGGAGAACACTGAAAACTCAGTGGATTCAAAATCCATTAA
AAATTTGGAACCAAAGATCATACATGGAAGCGAATCAATGGACTCTGGAATATCCCTGGACAACAGIT
ATAAAATGGATTATCCTGAGATGGGTTTATGTATAATAATTAATAATAAGAATTTTCATAAAAGCACT
GGAATGACATCTCGGTCTGGTACAGATGTCGATGCAGCAAACCTCAGGGAAACATTCAGAAACTTGAA
ATATGAAGTCAGGAATAAAAATGATCTTACACGTGAAGAAATTGTGGAATTGATGCGTGATGTTTCTA
AAGAAGATCACAGCAAAAGGAGCAGTTTTGTTTGTGTGCTTCTGAGCCATGGTGAAGAAGGAATAATT
TTTGGAACAAATGGACCTGTTGACCTGAAAAAAATAACAAACTTTTTCAGAGGGGATCGTTGTAGAAG
TCTAACTGGAAAACCCAAACTTTTCATTATTCAGGCCTGCCGTGGTACAGAACTGGACTGTGGCATTG
AGACAGACTAAGTATAAGAAGGAGATATACATATGAGTGGTGTTGATGATGACATGGCGTGTCATAAA
ATACCAGTGGAGGCCGACTTCTTGTATGCATACTCCACAGCACCTGGTTATTATTCTTGGCGAAATTC
AAAGGATGGCTCCTGGTTCATCCAGTCGCTTTGTGCCATGCTGAAACAGTATGCCGACAAGCTTGAAT
TTATGCACATTCTTACCCGGGTTAACCGAAAGGTGGCAACAGAATTTGAGTCCTTTTCCTTTGACGCT
ACTTTTCATGCAAAGAAACAGATTCCATGTATTGTTTCCATGCTCACAAAAGAACTCTATTTTTATCA
CGAACAAAAACTCATCTCAGAAGAGGATCTGTAA
Large subunit sequence (DNA sequence)
(SEQ ID NO: 201)
ATGGACTACAAAGACGATGACGACAAGATGGAGAACACTGAAAACTCAGTGGATTCAAAATCCATTAA
AAATTTGGAACCAAAGATCATACATGGAAGCGAATCAATGGACTCTGGAATATCCCTGGACAACAGTT
ATAAAATGGATTATCCTGAGATGGGTTTATGTATAATAATTAATAATAAGAATTTTCATAAAAGCACT
GGAATGACATCTCGGTCTGGTACAGATGTCGATGCAGCAAACCTCAGGGAAACATTCAGAAACTTGAA
ATATGAAGTCAGGAATAAAAATGATCTTACACGTGAAGAAATTGTGGAATTGATGCGTGATGTTTCTA
AAGAAGATCACAGCAAAAGGAGCAGTTTTGTTTGTGTGCTTCTGAGCCATGGTGAAGAAGGAATAATT
TTTGGAACAAATGGACCTGTTGACCTGAAAAAAATAACAAACTTTTTCAGAGGGGATCGTTGTAGAAG
TCTAACTGGAAAACCCAAACTTTTCATTATTCAGGCCTGCCGTGGTACAGAACTGGACTGTGGCATTG
AGACAGACTAA
Large subunit (protein sequence)
SEQ ID NO: 202)
MDYKDDDDKMENTENSVDSKSIKNLEPKIIHGSESMDSGISLDNSYKMDYPEMGLCIIIN
NKNFHKSTGMTSRSGTDVDAANLRETFRNLKYEVRNKNDLTREEIVELMRDVSKEDHS
KRSSFVCVLLSHGEEGIIFGTNGPVDLKKITNFFRGDRCRSLTGKPKLFIIQACRGTELDC
GIETD
Small subunit (DNA sequence)
SEQ ID NO: 203)
ATGAGTGGTGTTGATGATGACATGGCGTGTCATAAAATACCAGTGGAGGCCGACTTCTTGTATGCATA
CTCCACAGCACCTGGTTATTATTCTTGGCGAAATTCAAAGGATGGCTCCTGGTTCATCCAGTCGCTTT
GTGCCATGCTGAAACAGTATGCCGACAAGCTTGAATTTATGCACATTCTTACCCGGGTTAACCGAAAG
GTGGCAACAGAATTTGAGTCCTTTTCCTTTGACGCTACTTTTCATGCAAAGAAACAGATTCCATGTAT
TGTTTCCATGCTCACAAAAGAACTCTATTTTTATCACGAACAAAAACTCATCTCAGAAGAGGATCTGT
AA
Small subunit (protein sequence)
SEQ ID NO: 204)
MSGVDDDMACHKIPVEADFLYAYSTAPGYYSWRNSKDGSWFIQSLCAMLKQYADKLE
FMHILTRVNRKVATEFESFSFDATFHAKKQIPCIVSMLTKELYFYHEQKLISEEDL

After bacterial delivery via invasion and lysis, the anti-actin nanobody was bound to cellular actin (FIG. 4A), demonstrating specific targeting of an intracellular protein. As potential therapeutic proteins, delivery of both NIPP1-CD and CT Casp-3 caused more cell death than controls (P<0.001; FIG. 4B, left). Induced death was dependent on invasion and protein delivery (FIG. 4B, right). When administered to microfluidic tumors devices, ID Salmonella delivering NIPP1-CD (P<0.05) and CT Casp-3 (P<0.01) caused cell death that increased with time as bacteria invaded the tumor masses (FIG. 4C).

Delivery of CT Casp-3 was effective against both liver cancer and triple-negative breast cancer in mice (FIG. 4D-E). After 14 days of treatment, delivery to BALB/c mice reduced the volume of 4T1 mammary tumors two times more than controls (P<0.05, FIG. 4D). Administration of ID Salmonella with CT Casp-3 significantly reduced the volume of liver Hepa 1-6 tumors in C57L/J mice (P<0.001; FIG. 4E, left) and reduced tumor growth rate 28 times (P<0.05; FIG. 4E, middle), which is equivalent to an increase in doubling time from 5 to 148 days. Tumor volume reduced in two mice for over 50 days, and survival increased significantly compared to bacterial controls (P<0.05, FIG. 4E right). Treatment with CT Casp-3 completely eliminated the tumor from one mouse, which was disease free for over 124 days.

### Conclusion

Described herein is an autonomous, intracellular Salmonella vehicle that efficiently delivers properly folded and active proteins into cells. This bacterial strain is safe, eliminates tumors and increases survival. The engineered gene circuits produce protein drugs, cause hyper-invasion (flhDC) and trigger bacterial lysis after cell invasion. Because the system is autonomous, it does not require intervention and is self-timing. Protein delivery is triggered at the most opportune time for individual bacteria, ensuring that proteins are deposited inside cells and not in the extracellular environment. The accumulation of ID Salmonella in different cell types in tumors (FIGS. 1D&G), suggests that this system could be used to deliver proteins to non-cancerous tumor-associated cells, e.g., macrophages or endothelial cells.

Coupled together, two essential qualities of ID Salmonella enable the use of protein drugs that are currently not feasible. Intracellular Salmonella delivery (1) transports intact, functional proteins across the cell membrane; and preferential tumor accumulation (2) maintains safety for protein drugs that would act broadly against healthy cells. Both NIPP1-CD and CT Casp-3 have exclusively intracellular targets and would be toxic if delivered systemically. The specific accumulation of ID Salmonella eliminates these problems by focusing therapy specifically on the intracellular environment of tumors (FIGS. 1C and 3E).

The use of ID Salmonella to deliver CT Casp-3 can address the need for an effective treatment for unresectable hepatocellular carcinoma (HCC). No curative treatment currently exists for the 840,000 patients who are diagnosed with HCC annually (28, 29). Current therapies have toxic side effects and only modestly increase survival (29-31). Treatment with CT Casp-3 ID Salmonella can be curative (FIG. 4E) and is safer. Inclusion of the PsseJ-LysE circuit makes ID Salmonella self-limiting. The delivery bacteria lyse after cell invasion (FIG. 3F), reducing the possibility of unwanted infections.

Delivery with ID Salmonella enables targeting of inaccessible cancer pathways and will accelerate the generation of new cancer therapies. These therapies can be created by coding the genes for specific protein drugs into Salmonella expression cassettes. Nanobodies (FIG. 4A) can be designed that specifically inhibit pathways necessary for cancer survival and progression. Using bacteria to deliver proteins into cells will expand the number of accessible pathways, open up many targets across the soluble proteome for treatment, and increase the efficacy and safety of cancer treatment.

### BIBLIOGRAPHY

1 Uhlen, M. et al. Proteomics. Tissue-based map of the human proteome. Science 347, 1260419, doi:10.1126/science.1260419 (2015).
2 Hanahan, D. & Weinberg, R. A. Hallmarks of Cancer: The Next Generation. Cell 144, 646-674, doi:10.1016/j.cell.2011.02.013 (2011).
3 Ibarra, J. A. et al. Induction of Salmonella pathogenicity island 1 under different growth conditions can affect Salmonella-host cell interactions in vitro. Microbiology 156, 1120-1133, doi:10.1099/mic.0.032896-0 (2010).
4 Knodler, L. A. et al. Dissemination of invasive Salmonella via bacterial-induced extrusion of mucosal epithelia. Proc Natl Acad Sci USA 107, 17733-17738, doi:10.1073/pnas.1006098107 (2010).
5 Liss, V. et al. Salmonella enterica Remodels the Host Cell Endosomal System for Efficient Intravacuolar Nutrition. Cell Host Microbe 21, 390-402, doi:10.1016/j.chom.2017.02.005 (2017).
6 Krieger, V. et al. Reorganization of the endosomal system in Salmonella-infected cells: the ultrastructure of Salmonella-induced tubular compartments. PLoS Pathog 10, e1004374, doi:10.1371/journal.ppat.1004374 (2014).
7 Rajashekar, R., Liebl, D., Seitz, A. & Hensel, M. Dynamic remodeling of the endosomal system during formation of Salmonella-induced filaments by intracellular Salmonella enterica. Traffic 9, 2100-2116, doi:10.1111/j.1600-0854.2008.00821.x (2008).
8 Galan, J. E. Salmonella interactions with host cells: type III secretion at work. Annu Rev Cell Dev Biol 17, 53-86, doi:10.1146/annurev.cellbio.17.1.53 (2001).
9 Eisele, N. A. et al. Salmonella require the fatty acid regulator PPARdelta for the establishment of a metabolic environment essential for long-term persistence. Cell Host Microbe 14, 171-182, doi:10.1016/j.chom.2013.07.010 (2013).
10 Knuff, K. & Finlay, B. B. What the SIF Is Happening—The Role of Intracellular Salmonella-Induced Filaments. Front Cell Infect Microbiol 7, 335, doi:10.3389/fcimb.2017.00335 (2017).
11 Zhang, K. et al. Minimal SPI1-T3SS effector requirement for Salmonella enterocyte invasion and intracellular proliferation in vivo. PLoS Pathog 14, e1006925, doi:10.1371/journal.ppat.1006925 (2018).
12 Forbes, N. S., Munn, L. L., Fukumura, D. & Jain, R. K. Sparse initial entrapment of systemically injected Salmonella typhimurium leads to heterogeneous accumulation within tumors. Cancer Res 63, 5188-5193 (2003).
13 Low, K. B. et al. Lipid A mutant Salmonella with suppressed virulence and TNFalpha induction retain tumor-targeting in vivo. Nature biotechnology 17, 37-41, doi:10.1038/5205 (1999).
14 Zheng, J. H. et al. Two-step enhanced cancer immunotherapy with engineered Salmonella typhimurium secreting heterologous flagellin. Sci. Transl. Med. 9, 10, doi:10.1126/scitranslmed.aak9537 (2017).
15 Zhao, M. et al. Tumor-targeting bacterial therapy with amino acid auxotrophs of GFP-expressing Salmonella typhimurium. Proceedings of the National Academy of Sciences of the United States of America 102, 755-760, doi:10.1073/pnas.0408422102 (2005).
16 Morrissey, D., O'Sullivan, G. C. & Tangney, M. Tumour Targeting with Systemically Administered Bacteria. Current Gene Therapy 10, 3-14, doi:10.2174/156652310790945575 (2010).
17 Ganai, S., Arenas, R. B., Sauer, J. P., Bentley, B. & Forbes, N. S. In tumors Salmonella migrate away from vasculature toward the transition zone and induce apoptosis. Cancer Gene Ther 18, 457-466, doi:10.1038/cgt.2011.10 (2011).
18 Forbes, N. S. Engineering the perfect (bacterial) cancer therapy. Nature Reviews Cancer 10, 785-794, doi:10.1038/nrc2934 (2010).
19 Kong, W., Clark-Curtiss, J. & Curtiss, R., 3rd. Utilizing Salmonella for antigen delivery: the aims and benefits of bacterial delivered vaccination. Expert Rev Vaccines 12, 345-347, doi:10.1586/erv.13.7 (2013).
20 Raman, V., Van Dessel, N., O'Connor, O. M. & Forbes, N. S. The motility regulator flhDC drives intracellular accumulation and tumor colonization of Salmonella. J Immunother Cancer 7, 44, doi:10.1186/s40425-018-0490-z (2019).
21 Walsh, C. L. et al. A multipurpose microfluidic device designed to mimic microenvironment gradients and develop targeted cancer therapeutics. Lab on a chip 9, 545-554, doi:10.1039/b810571e (2009).
22 Finn, C. E., Chong, A., Cooper, K. G., Starr, T. & Steele-Mortimer, O. A second wave of Salmonella T3SS1 activity prolongs the lifespan of infected epithelial cells. PLoS Pathog 13, e1006354, doi:10.1371/journal.ppat.1006354 (2017).
23 Beuzon, C. R. et al. Salmonella maintains the integrity of its intracellular vacuole through the action of SifA. EMBO J 19, 3235-3249, doi:10.1093/emboj/19.13.3235 (2000).
24 Ruiz-Albert, J. et al. Complementary activities of SseJ and SifA regulate dynamics of the Salmonella typhimurium vacuolar membrane. Mol. Microbiol. 44, 645-661, doi:10.1046/j.1365-2958.2002.02912.x (2002).
25 Chatterjee, J. et al. Development of a peptide that selectively activates protein phosphatase-1 in living cells. Angewandte Chemie (International ed 51, 10054-10059, doi:10.1002/anie.201204308 (2012).
26 Slee, E. A., Adrain, C. & Martin, S. J. Executioner caspase-3, -6, and -7 perform distinct, non-redundant roles during the demolition phase of apoptosis. J Biol Chem 276, 7320-7326, doi:10.1074/jbc.M008363200 (2001).
27 Walsh, J. G. et al. Executioner caspase-3 and caspase-7 are functionally distinct proteases. Proc Natl Acad Sci USA 105, 12815-12819, doi:10.1073/pnas.0707715105 (2008).
28 Siegel, R. et al. Cancer treatment and survivorship statistics, 2012. CA Cancer J Clin 62, 220-241, doi:10.3322/caac.21149 (2012).
29 Raza, A. & Sood, G. K. Hepatocellular carcinoma review: current treatment, and evidence-based medicine. World J Gastroenterol 20, 4115-4127, doi:10.3748/wjg.v20.i15.4115 (2014).
Keating, G. M. & Santoro, A. Sorafenib: a review of its use in advanced hepatocellular carcinoma. Drugs 69, 223-240, doi:10.2165/00003495-200969020-00006 (2009).
31 Vogl, T. J. et al. [Transarterial chemoembolization (TACE) in hepatocellular carcinoma: technique, indication and results]. Rofo 179, 1113-1126, doi:10.1055/s-2007-963285 (2007).

### Example II

### Introduction

Intracellularly targeted, macromolecular therapies present an opportunity for treatment of cancer. The mammalian proteome consists of 60% intracellular protein while only 30% are surface associated and extracellularly exposed (1). However, macromolecules face tumor specificity, distribution, cell internalization and endosomal release barriers (2). An improved drug delivery system is needed to circumvent these delivery limitations and increase therapeutic efficacy of intracellularly active therapies. Salmonella are ideally suited for tumor selective intracellular protein delivery. Salmonella colonize tumors with high specificity, invade, and deliver protein therapies selectively inside tumor cells. Herein the discovery that flhDC expression is crucial for protein delivery into tumor cells with Salmonella has been reported. To this end, it was sought to determine the mechanisms by which flhDC expression enables intracellular therapeutic delivery in vivo. The unique mechanisms by which engineered Salmonella expressing flhDC developed resistance to intracellular therapeutic delivery was also assessed. Understanding these mechanisms help create improved tumor targeted, intracellular delivery strains of Salmonella.

Typhoidal strains of Salmonella that systemically infect humans carefully modulate flagellar expression in vivo. The typhoidal bacteria that disseminate systemically infect humans implement genetic programs to downregulate expression of the flagellar synthesis regulator (3-5), flhDC, in the blood (6, 7). One reason for this is because flagellin is a TLR5/NLRC4 agonist that strongly activates an anti-microbial immune response (8, 9). However, in tumor tissue, intracellular invasion and delivery into cancer cells requires activation of the Salmonella transcription factor, flhDC (10). Therefore, developing a method to control flhDC activity in engineered Salmonella is necessary to enable high levels of therapeutic delivery in tumors.

Modulation of flhDC activity within Salmonella has significant implications in determining tumor selectivity and reducing systemic virulence. Unlike tumors, clearance organs like the liver and spleen, have high concentrations of functional immune cells that mount strong responses upon pathogenic insult. The liver is a vital clearance organ and an essential site specific for immune mediated Salmonella clearance (11). The motility regulator, flhDC, regulates flagellar expression but is also a broad regulator of Salmonella lifestyle and virulence (10, 12, 13). Flagellar expression within Salmonella in macrophages or epithelial cells causes excessive, NLRC4 inflammasome dependent, pyroptosis. Salmonella hijack this inflammatory pathway to overexaggerate the anti-microbial response both in macrophages and within the gut, causing immune dysfunction (14, 15). Since the liver contains large quantities of Kupfer cells, flagellated Salmonella can cause significant pyroptosis in these liver specific macrophages. While pyroptosis is required in limited quantities to eliminate pathogens, flagellated Salmonella cause high levels of pyroptosis that render the anti-microbial immune response dysfunctional (14, 16). Macrophages are more effective at clearing Salmonella expressing lower levels of flhDC due to reduced flagellar expression, limited pyroptosis resulting in less immune dysfunction (16). Since tumors do not share the same level of immune function, low flagellin expression does not affect tumor colonization (17).

Upon invasion into a cell, there are two existing mechanisms by which therapy is delivered into the cytosol by Salmonella: (1) The bacteria invade, escape the intracellular vacuole, rupture, and deliver therapy into the cytosol (18-20) or (2) the bacteria are genetically engineered to lyse and deliver therapy from within the Salmonella containing vacuole into the cytosol. Several variants of cytosolic bacteria (ΔsifA Salmonella, Listeriolysin O expressing bacteria) have been used for therapeutic delivery into tumor cells (18-20). In scenario (1), therapeutic delivery would require the bacteria to reside in the cytoplasm of a cancer cell and lyse spontaneously without any control. This mechanism would depend on ubiquitin dependent degradation (21) of the bacteria and subsequent cytosolic release of the therapy. In addition, cytoplasmic pathogens are known to strongly activate NF-kB signaling and initiate innate immune responses to clear the bacteria (9, 21, 22). Therefore, a high presence of cytosolic Salmonella is detrimental for immune evasion.

Salmonella have evolved to reside within an intracellular vacuole which confers protection to the bacteria inside cells (23, 24). The bacteria modify the vacuole to confer protection against degradation and clearance (25, 26). In addition, vacuolar residence seems to be especially important for bacteria in systemic circulation as demonstrated by Salmonella Typhi. The spi-2 protein, SseJ, is required for Salmonella to escape the SCV (27). Salmonella Typhimurium, which express SseJ, are localized to the gastrointestinal tract in humans (28). Salmonella Typhi, which lacks SseJ (29), is efficient at escaping the gastrointestinal tract into systemic circulation (30). Moreover, Salmonella Typhi only expresses typhoid toxin intracellularly within the SCV (31, 32). These critical between Salmonella Typhi and Salmonella Typhimurium suggest that vacuolar residence is imperative to increase bacterial fitness in vivo.

Understanding the dynamics between vacuolar and cytosolic Salmonella expressing flhDC will aid in engineering an intracellular delivery strain of Salmonella. Herein we have shown that intracellular lysis of engineered Salmonella occurs in a vacuole. However, flagellated, intracellular Salmonella have a significant cytosolic presence (12). Intracellular Invasion is driven by flhDC and T3SS1 activity (10, 12, 33-35). Upon invading cells, Salmonella heavily modify the vacuoles in which they reside (24, 36). In doing so, some bacteria rupture the vacuole and escape (37, 38). Normally, the intravacuolar bacteria also downregulate flagellar expression through ssrB directed suppression of flhDC (39) (the ssrB protein is considered a master regulator of SPI-2 expression (33)). However, flagellated, cytosolic Salmonella have abrogated T3SS2 activity due to vacuolar escape (12). As shown herein, T3SS2 activity is needed to enable intracellular lysis and delivery of protein with therapeutic Salmonella.

Provided herein is a showing of how controlled expression of flhDC could improve tumor colonization and therapeutic delivery in vivo as compared to existing delivery strategies. Further the mechanism is eluicidated of flhDC induced resistance to therapeutic delivery and a genetic engineering strategy to rescue therapeutic delivery of the Salmonella strain. It was hypothesized that flhDC expression selectively within intratumoral bacteria is important for increasing tumor specificity, colonization and protein delivery to a spatially distributed set of tumor cells. It was further hypothesized that engineered Salmonella inducibly expressing flhDC could deliver more protein intracellularly compared to exclusively cytosolic Salmonella. It was also hypothesized that flhDC activity enabled lysis resistance in engineered Salmonella but could be rescued. To test these hypotheses, cell-based assays, tumor-on-a-chip models, and in vivo experiments were employed to quantitatively understand the mechanisms underlying intracellular therapeutic delivery with engineered Salmonella. Discovering the key mechanisms governing therapeutic delivery with Salmonella would address limitations with current delivery methods and provide a foundation to robustly improve delivery efficiency of the engineered bacteria for a wide variety of cancers.

### Materials and Methods

### Bacterial Cultures

All bacterial cultures (both Salmonella and DH5α) were grown in LB (10 g/L sodium chloride, 10 g/L tryptone and 5 g/L yeast extract). Resistant strains of bacteria were grown in the presence of carbenicllin (100 μg/ml), chloramphenicol (33 μg/ml), kanamycin (50 μg/ml) and/or 100 μg/ml of DAP.

### Cloning

One of three plasmids were used in all experiments. The first plasmid, P1, was created by cloning the flhDC gene into the PBAD his-myc plasmid (Invitrogen; catalog #V430-01). Primers vr46 and vr47 were used to PCR the flhDC gene from VNP20009 genomic DNA. The PCR product was digested with NcoI and XhoI and DpnI (NEB, catalog #s R0193S, R0146S and R0176L). The PBAD-his-myc backbone was digested with NcoI, XhoI and calf intestinal phosphatase (NEB, catalog #M0290). A PCR cleanup column (Zymo Research) was used to clean up both products. 50 ng of digested vector backbone and 500 ng of digested PCR product were ligated together using T4 DNA ligase (NEB). The ligated product was transformed into DH5a E. Coli. Positive transformants were confirmed by sequencing (Plasmid P1a). To add the plac-GFP-myc genetic circuit to the plasmid, plasmid P1a was PCR amplified using primers vr385 and vr386. The plac-GFP-myc genetic circuit was PCR amplified from a previously generated plasmid (40) using primers vr394 and vr395. Both PCR products were DpnI digested. 50 ng of P1a PCR product and 500 ng of were ligated together using a 2×Hifi DNA assembly master mix (NEB). The resulting product was transformed into DH5a E. Coli and the complete P1b plasmid was purified from positive colonies. To create complete plasmid P1, PCR was used to amplify the P1b backbone using primers vr426 and vr427. The ASD gene was amplified from a previously generated plasmid, PCS2 (40) using primers vr424 and vr425. 50 ng of the P1b PCR product and 500 ng of the ASD PCR product were ligated together using 2×Hifi DNA assembly master mix. The resulting ligation was transformed into chemically competent DH5a E. Coli. Complete, P1 plasmid was purified from colonies screening positive for GFP, ASD and PBAD-flhDC.

To create plasmid P2, plasmid P1 was PCR amplified using primers vr396 and vr397. The psseJ-lysinE genetic circuit was amplified from synthesized DNA (Genscript) using primers vr398 and vr399. The two PCR products were DpnI digested and purified using PCR clean up columns (Zymo Research). 50 ng of backbone PCR and 500 ng of psseJ-lysinE PCR product was used in a ligation reaction with 2×Hifi assembly master mix (NEB) to create plasmid, P2a. Plasmid was purified from colonies that screened positive for plasmid assembly for downstream applications. To create complete P2, plasmid P2a was PCR amplified using primers vr426 and vr427. The ASD gene was amplified as previously described using primers vr424 and vr425. Both PCR products were DpnI digested and purified using a PCR clean up column as previously described. 50 ng of the P2a PCR product was ligated together with 500 ng of the ASD PCR product using 2×Hifi DNA assembly master mix. The resulting ligation was transformed into DH5a E. Coli and complete P2 plasmid was purified from colonies screening positive for GFP, ASD, PBAD-flhDC and sseJ-lysinE.

To create plasmid P3 (sseJ-GFP-myc+PBAD-flhDC), plasmid P1a was PCR amplified using primers vr271 and vr272. The sseJ-GFP-myc genetic circuit was PCR amplified from a previously generated plasmid (10) using primers vr269 and vr270. The resulting PCR products were DpnI digested and purified using PCR clean up columns. 50 ng of the P1a backbone and 500 ng of the psseJ-GFP-myc PCR products were ligated together using 2×Hifi DNA assembly master mix. The resulting ligations were transformed into DH5a E. Coli. Complete, P3 plasmid was purified from colonies that screened positive for psseJ-GFP-myc and PBAD-flhDC for downstream application.

TABLE 1
Primers for deletion mutants

| Name | Primer sequence | Gene | Template |
|---|---|---|---|
| vr121 | FOFZCACGGGGTGCGGCTACGTCGCACAAA | flhD forward | pkd4 |
| | AATAAAGTTGGTTATTCTGGGTCTTGAGCG | | |
| | ATTGTGTAGGC SEQ ID NO: 205) | | |
| vr309 | EOEFATCCTGAGTCAAACGGGTGATCGTCT | flhD reverse | pkd4 |
| | GATGATCGTCAAACCGGAAAAATTAGCCAT | | |
| | GGTCCATATGAATATC SEQ ID NO: 206) | | |
| vr266 | FZEFAAAATGTTGGTTTTATCGGCTGGCGCG | asd forward | pkd3 |
| | GAATGGTCGGCTCTGTTCTGTCTTGAGCGAT | | |
| | TGTGTAGGC SEQ ID NO: 207) | | |
| vr268 | OZFOGCCAACTGGCGCAGCATTCGACGCAG | asd reverse | pkd3 |
| | CGGCTCGGCGGCGCCCCATAAATTAGCCAT | | |
| | GGTCCATATGAATATC SEQ ID NO: 208) | | |
| vr318 | FZEFGTAATCTTAGCGGTACCGATAAAAGC | fliGHI | pkd3/pkd4 |
| | GTCATTTTGTTGATGACCATGTCTTGAGCGA | forward | |
| | TTGTGTAGGC SEQ ID NO: 209) | | |
| vr319 | FFFFTTAAATCGAGCGCCTGCAGAGAGTCCT | fliGHI | pkd3/pkd4 |
| | CCCAGTCGGCCCGTTCAAAAATTAGCCATG | reverse | |
| | GTCCATATGAATAT SEQ ID NO: 210) | | |
| vr432 | FZEOCATTGAGTGTTGGACAGGGTTATTTCA | sseJ forward | pkd4 |
| | CATCATCTATCAGTTCTGAGTCTTGAGCGAT | | |
| | TGTGTAGGC SEQ ID NO: 211) | | |
| vr433 | ZZFZTCAGTGGAATAATGATGAGCTATAAA | sseJ reverse | pkd4 |
| | ACTTTCTAACATTATGGCAAAATTAGCCAT | | |
| | GGTCCATATGAATATC SEQ ID NO: 212) | | |
| vr434 | FZEOCGATTACTATAGGGAATGGTTTTTTAA | sifA forward | pkd4 |
| | AAAGTGAAATCCTTACCAAGTCTTGAGCGA | | |
| | TTGTGTAGGC SEQ ID NO: 213) | | |
| vr435 | ZZFZAAAAAACAACATAAACAGCCGCTTTG | sifA reverse | pkd4 |
| | TTGTTCTGAGCGAACGTGTAAATTAGCCAT | | |
| | GGTCCATATGAATATC SEQ ID NO: 214) | | |

TABLE 2
Primers for plasmid construction

| Name | Sequence | Description |
|---|---|---|
| vr46 | AAAAAACCATGGGTTAATAAAAGGAGGAATATAT | flhDC |
| | ATGCATACATCCGAGTTGCTAAAACA | forward |
| | SEQ ID NO: 215) | |
| vr47 | AAAAAACTCGAGAAAAATTAAACAGCCTGTTCGA | flhDC |
| | TCTGTTCAT SEQ ID NO: 216) | reverse |
| vr394 | CCGCATAGTTAAGCCAGTATACATTTACACTTTAT | pLacGFP |
| | GCTTCCGGCTCGTATAATAAAAAAAAAAGGAGGA | backbone |
| | AAAAAAATGAGTAAAGGAGAAGAACTTTTCA SEQ | forward |
| | ID NO: 217) | |
| vr395 | TCACGTAGCGATAGCGGAGTTACAGATCCTCTTCT | pLacGFP |
| | GAGATGAGTTTTTGTTCTTTGTATAGTTCATCCAT | backbone |
| | GCCAT SEQ ID NO: 218) | reverse |
| vr385 | CTCCGCTATCGCTACGTGA SEQ ID NO: 219) | Pla |
| | | backbone |
| | | forward |
| vr386 | TGTATACTGGCTTAACTATGCGG | Pla |
| | | backbone |
| | SEQ ID NO: 220) | reverse |
| vr424 | GCTTGTCTGCTCCCGGCATCGTACGTTTTCGTTCC | asd forward |
| | ATTGG SEQ ID NO: 221) | |
| vr425 | AGACGGTCACAGCTTGTCTGTATCTGCGTTTACTC | asd reverse |
| | CTGTATTAC SEQ ID NO: 222) | |
| vr426 | ACAGACAAGCTGTGACCGTCT | P1b or P2a |
| | SEQ ID NO: 223) | backbone |
| | | forward |
| vr427 | ATGCCGGGAGCAGACAAGC SEQ ID NO: 224) | P1b or P2a |
| | | backbone |
| | | reverse |
| vr269 | CGCAGCGAGTCAGTGAGCACATGTCACATAAAAC | Pssej- |
| | ACTAGCACT SEQ ID NO: 225) | GFP-myc |
| | | forward |
| vr270 | CGCACAGATGCGTAAGGAGAATTACAGATCCTCT | Pssej- |
| | TCTGAGATGAGTTTTTGTTCTTTGTATAGTTCAT | GFP-myc |
| | CCATGCCATG SEQ ID NO: 226) | reverse |
| vr271 | GCTCACTGACTCGCTGCG SEQ ID NO: 227) | Pla backbone |
| | | reverse |
| vr272 | TTCTCCTTACGCATCTGTGCG | Pla backbone |
| | SEQ ID NO: 228) | forward |
| vr398 | ATCTGTGCGGTATTTCACACCACATGTCACATAAA | Pssej-LysE |
| | ACACTAGCACT SEQ ID NO: 229) | forward |
| vr399 | TACTGAGAGTGCACCATATGCTCACTCCTTCCGCA | Pssej-LysE |
| | CGTAATTT SEQ ID NO: 230) | reverse |
| vr396 | GCATATGGTGCACTCTCAGTA | P1 backbone |
| | SEQ ID NO: 231) | forward |
| vr397 | GGTGTGAAATACCGCACAGAT | P1 backbone |
| | SEQ ID NO: 232) | reverse |

TABLE 3
Plasmids

| | | | Gene | Gene | |
|---|---|---|---|---|---|
| No. | Name | Origin | Maintenance | Circuits | Purpose |
| P1 | flhDC re- | ColE1 | Amp | PBAD-flhDC | Re-expresses flhDC; |
| | expressing | | ASD | Plac-GFP-myc | Constitutively expresses |
| | | | | | GFP |
| P2 | Intracellular lysis | ColE1 | AMP | PsseJ-LysE | Lyses after invasion; |
| | and induced | | ASD | PBAD-flhDC | Re-expresses flhDC; |
| | invasion | | | Plac-GFP-myc | Constitutively expresses |
| | | | | | GFP |
| P3 | flhDC reporting | ColE1 | Amp | PBAD-flhDC | Measures invasion after |
| | | | | PsseJ-GFP-myc | flhDC re-expression |

### Strains

All engineered strains were based on VNP20009 and strain details can be found in following table.

Genetic knockouts were created using a modified lambda red recombination procedure (41, 42). The master gene editing strain was created by transforming the plasmid containing the required lambda phage genes, pkd46, into VNP20009 using electroporation.

Six genomic knockout strains of Salmonella were created. Three of the knockouts were created by growing Salmonella containing pkd46 to an optical density of 0.1 at which point the bacteria were supplemented with 20 mM arabinose to induce expression of lambda genes. When the bacteria reached an optical density of 0.8, 1 microgram of DpnI digested PCR product amplified from pkd4 (vr121/vr309 for ΔflhD,vr318/vr319 for ΔfliGHI, vr432/vr433 for ΔsseJ, vr434/vr435 for ΔsifA) was transformed into the Salmonella through electroporation. Bacteria was recovered in LB for 2 hours at 37° C. and plated on agar plates containing 50 micrograms/ml of kanamycin. Resulting transformants were screened for insertion using antibiotic selection and junction PCR to confirm correct location of genomic deletion. Successful knockouts were then grown at 43° C. to cure the knockout strains of the pkd46 plasmid.

To create the ΔflhD+ΔfliGHI knockout, the above strain of ΔflhD was retransformed with pkd46 through electroporation, grown to an OD of 0.1 and induced with 20 mM arabinose until the bacteria grew to an OD of 0.8. The fliGHI knockout PCR product was amplified from pkd3 using the primers, vr266 and vr268. The PCR products were DpnI digested and 1 microgram was transformed into the lambda induced ΔflhD strain using electroporation. The bacteria were recovered in LB with 100 micrograms/ml for 2 hours at 37° C. and plated on agar plates containing 33 micrograms/ml of chloramphenicol. Successful transformants were screened as previously described and grown on LB containing 33 micrograms/ml of chloramphenicol overnight at 43° C. to cure the bacteria of pkd46.

The plasmids created were transformed into the relevant strains using electroporation. These strains are listed in Table 3.

### Mouse Models

Six week old Balb/C mice from Jackson Laboratories were injected subcutaneously with 1×10W 4T1 tumor cells on the hind flank. Once tumors reached 500 mm³, mice were intravenously injected with either saline or bacteria. Either twenty-four or ninety-six hours after bacterial administration, mice were sacrificed, and tumors, livers and spleens were excised for downstream analysis.

### In Vivo Tumor and Liver Colonization of Salmonella

To quantify tumor and liver colonization five groups of five Balb/C mice containing subcutaneous 4T1 tumors (˜500 mm³) were intravenously injected via the tail vein with either parental, ΔflhD, ΔfliGHI, or ΔflhD+ΔfliGHI Salmonella. Ninety-six hours after bacterial administration, tumors and livers were excised and homogenized in two volumes (w/v) of sterile PBS. Organ slurries were serially diluted 10-fold, four times for livers and eight time for tumors. 200 ul of each dilution was plated on agar containing the appropriate antibiotic. After drying, plates were incubated overnight at 37 degrees Celsius. Plates containing between 10 and 100 colonies were counted to determine bacterial colonization levels in either the tumor or liver.

### Immunohistochemistry

Excised tumor sections were fixed in 10% formalin for 3 days. Fixed tumor samples were then stored in 70% ethanol for 1 week. Tumor samples were embedded in paraffin and sectioned into 5 μm sections. Deparaffinization was performed by washing the sectioned tissue three times in 100% xylene, twice in 100% ethanol, once in 95% ethanol, once in 70% ethanol, once in 50% ethanol, and once in DI water. Each wash step was performed for 5 minutes. Antigen retrieval was performed by incubating the tissue sections in 95° C., 20 mM sodium citrate (pH 7.6) buffer for 20 minutes. Samples were left in sodium citrate buffer until the temperature reduced to 40° C. Samples were then rehydrated with two quick (<1 minute) rinses in DI water followed by one five-minute wash in TBS-T.

Prior to staining, tissue sections were blocked with Dako blocking buffer (Dako) for one hour. Tissue sections were stained to identify Salmonella and GFP with 1:100 dilutions of (1) FITC-conjugated rabbit anti-Salmonella polyclonal antibody (Abcam), and (2) either rat anti-myc monoclonal antibody (Chromotek) or rat anti-GFP monoclonal antibody (Chromotek) in Tris buffered saline with 0.1% Tween 20 (TBS-T) with 2% BSA (FisherScientific). Sections were washed three times in TBS-T w/ 2% BSA and incubated with Alexaflor-568 goat anti-rat secondary antibodies (ThermoFisher). After washing sections three times with TBS-T, 40 μl of prolong gold mountant with DAPI (ThermoFisher) and a cover slip were added to each slide. Slides were incubated at room temperature for 24 hours until the mountant solidified. Histological Detection of Intracellular delivery of GFP to cells in tumors with FID Salmonella

To identify and quantify GFP delivery to tumor cells, two groups of ten BALB/c mice with 4T1 tumors were injected with 2×10⁶ CFU of FID Salmonella. One group of mice was injected twice with arabinose intraperitoneally to induce flhDC expression while the other group was injected with saline as a control. Ninety-six hours after bacterial injection, mice were sacrificed and tumors, liver and spleens were excised. Tumors were cut in half. One half was fixed and stained for imaging as described in the immunohistochemistry section.

### Cell Culture

Two cancer cell lines were used: 4T1 murine breast carcinoma cells and LS174T human colorectal carcinoma cells (ATCC, Manassas, VA). All cancer cells were grown and maintained in Dulbecco's Minimal Eagle Medium (DMEM) containing 3.7 g/L sodium bicarbonate and 10% fetal bovine serum. For microscopy studies, cells were incubated in DMEM with 20 mM HEPES buffering agent and 10% FBS. To generate tumor spheroids, single cell suspensions of LS174T cells were transferred to PMMA-coated cell culture flasks (2 g/L PMMA in 100% ethanol, dried before use).

Microfluidic System to Quantify Intracellular Invasion Distribution of flhDC Induced Salmonella

To quantify invasion into tumor masses, engineered Salmonella were administered to tumor-on-a-chip devices developed in our laboratory (43, 44). Microfluidic tumor-on-a-chip devices were fabricated using negative tone photoresist and PDMS based soft lithography. Master chips were constructed by spin coating a layer of SU-8 2050 onto a silicon wafer at 1250 RPM for 1 minute. This speed corresponded to an SU-8 2050 thickness of 150 μm. The silicon wafer was baked at 65° C. for 5 minutes followed by 95° C. for 30 minutes. Microfluidic designs printed on a high-resolution transparency were placed over the silicon wafer in a mask aligner. The silicon wafer with the overlaid mask was exposed to UV light (22 J/cm²) for 22 seconds. Silicon wafers were baked for 5 minutes at 65° C. followed by 95° C. for 12 minutes. Wafers were then developed in PGMEA developing solution for 10 minutes and/or until microfluidic features were microscopically distinct with sharp and defined edges.

Soft lithography was used to create the multilayer tumor on a chip device with 12 tumor chambers (two conditions with six chambers each). PDMS (Sylgard 184) at ratios of 9:1 and 15:1 were used for the channel and valve layers, respectively. The channel layer was placed on a spin coater for 1 minute at 220 rpm in order to achieve a PDMS thickness of 200 μm. The silicon wafers were degassed for 45 minutes to eliminate air bubbles in the PDMS. The silicon wafers were baked at 65 degrees for approximately one hour or until both PDMS layers were partially cured. The top valve layer of PDMS was cut and removed from the silicon wafer and aligned on top of the channel layer using a stereomicroscope. The combined layers were baked for one hour at 95° C. in order to covalently bind the two layers. The multilayered PDMS device and a glass slide was plasma treated in a plasma cleaner (Harrick) for 2.5 minutes. Valves were pneumatically actuated with a vacuum pump and the PDMS was placed on the plasma treated glass slide. Valves were actuated until the device was ready for use.

The tumor-on-a-chip was sterilized with 10% bleach followed by 70% ethanol, each for one hour. Microfluidic chips were equilibrated with media (DMEM with 20 mM HEPES, pH 7.4) for one hour. Valve actuation was used to position tumor spheroids in the tumor chambers. Valves at the rear of the chambers were opened while the efflux channel was closed. After the tumor masses were positioned, the valves were reset so that the rear valves were closed, and the influx and efflux channels were open.

Prior to administration to the device, flhDC reporting Salmonella were grown in LB with 20 mM arabinose to induce flhDC expression. These Salmonella have inducible flhDC (PBAD-flhDC) and produce GFP when intracellular (PsseJ-GFP). Control (flhDC−) Salmonella of the same strain were grown without arabinose. The bacteria were centrifuged and resuspended in culture medium (DMEM with 20 mM HEPES) at a density of 2×10⁷ CFU/ml. For the induced flhDC+condition, 20 mM arabinose was added to the medium. Bacteria-containing media (flhDC+ and flhDC−; n=6 chambers each) were perfused through the tumor-on-a-chip devices for one hour at 3 μm/min for a total delivery of 2×10⁶ CFU to each device. Bacterial administration was followed by bacteria-free media (with 20 mM HEPES) for 48 hours.

Devices were imaged at 30-minute intervals. Invasion was quantified at 31 h by measuring GFP expression by invaded bacteria in the tumor masses. Regions of interest were defined around the borders of the tumor masses. The extent of invasion was determined as the average GFP fluorescence intensity in each tumor mass. Intensities were normalized by the intensity of the average tumor mass administered control (flhDC−) Salmonella.

### Microscopy and Image Analysis

Samples were imaged on a Zeiss Axio Observer Z.1 microscope. Fixed cells on coverslips were imaged with a 100× oil immersion objective (1.4 NA). Tumor sections were images with 10× and 20× objectives (0.3 and 0.4 NA, respectively). Time lapse fluorescence microscopy of live cells in well plates and tumor-chip devices were housed in a humidified, 37° C. environment and imaged with 5×, 10×, 63× or 100× objectives (0.2, 0.3, 1.4 and 1.4 NA, respectively). Fluorescence images were acquired with either 480/525 or 525/590 excitation/emission filters. All images were background subtracted and contrast was uniformly enhanced. All immunocytochemistry image analysis was automated using computational code (MATLAB, Mathworks). Immunohistochemical imaging of bacterial distribution in tumors was automated using MATLAB. Intracellular protein delivery within mouse tumors was visually quantified.

### Infection Assays

For infection assays, cancer cells were grown on coverslips for fixed-cell imaging. For fixed imaging, glass coverslips were placed in 12-well plates and sterilized with UV light in a biosafety hood for 20 minutes. Mouse 4T1 were seeded on the coverslips at 40% confluency and incubated overnight in DMEM. Concurrently, Salmonella were grown to an optical density (OD; at 600 nm) of 0.8. After incubation, the Salmonella were added to the 4T1 cultures at a multiplicity of infection (MOI) of 10 and allowed to infect the cells for two hours. After this invasion period, the cultures were washed five times with 1 ml of phosphate buffered saline (PBS) and resuspended in 2 ml of DMEM with 20 mM HEPES, 10% FBS and 50 μg/ml gentamycin. The added gentamycin removes extracellular bacteria. After six hours of incubation, the media was removed, and the coverslips were fixed with 10% formalin in PBS for 10 minutes.

### Immunocytochemistry

Immunocytochemistry was used to obtain detailed images of Salmonella invaded into cancer cells grown on coverslips. After fixing the coverslips with formalin, they were blocked with staining buffer (PBS with 0.1% Tween 20, 1 mM EDTA, and 2% bovine serum albumin (BSA)) for 30 minutes. The Tween 20 in this buffer selectively permeabilizes mammalian cell membranes, while leaving bacterial membranes intact.

After permeabilization, coverslips were stained to identify Salmonella, released GFP, and vacuolar membranes with (1) rabbit anti-Salmonella polyclonal antibody (Abcam) or FITC-conjugated rabbit anti-Salmonella polyclonal antibody (Abcam) (2) rat anti-myc monoclonal antibody (Chromotek), and (3) rabbit anti-LAMP1 polyclonal antibody (Abcam), respectively. Three different staining combinations were used: (1) Salmonella alone; (2) Salmonella, released GFP and (3) Salmonella, released GFP and vacuoles.

For Salmonella alone staining (combination 1), coverslips were stained with FITC-conjugated anti-Salmonella antibody at 30° C. for one hour and washed three times with staining buffer.

For Salmonella, released GFP and vacuole staining (combination 2), coverslips were stained sequentially with anti-LAMP1 primary antibodies at 30° C. for one hour, and washed three times with staining buffer. Coverslips were incubated with Alexaflor-647 chicken anti-rabbit secondary antibodies (ThermoFisher) at a 1:200 dilution for one hour at 30° C. and washed four times with staining buffer. Coverslips were then stained with FITC-conjugated anti-Salmonella antibody and anti-myc primary antibody; and washed three times with staining buffer. Coverslips were incubated with Alexaflor-568 goat anti-rat secondary antibodies (ThermoFisher) at a 1:200 dilution for one hour at 30° C. to identify GFP.

After all staining, coverslips were washed three times with staining buffer and mounted to glass slides using 20 μl mountant with DAPI (ProLong Gold Antifade Mountant, Thermofisher). Mounted coverslips were cured overnight at room temperature.

### Quantification of Vacuolar Fraction, Extent of Invasion, and Lysis of Engineered Salmonella

To quantify what fraction of intracellular, flhDC expressing Salmonella were located within vacuoles, coverslips were infected with either the parental control strain of Salmonella or FID Salmonella as described in the infection assay section. Coverslips were then stained for LAMP1, Salmonella and nuclei as described in the immunocytochemistry section. Coverslips were imaged at 100× as described in the microscopy and image analysis section. Between ten and twenty cells from either the control group or FID treated group were analyzed. Salmonella either colocalized or bordered very closely by LAMP1 were defined as inside vacuoles. Salmonella that were not localized with LAMP1 closely bordering the bacteria were defined as cytosolic.

### Results

Controlling flhD Expression Improves Tumor Targeting of Salmonella

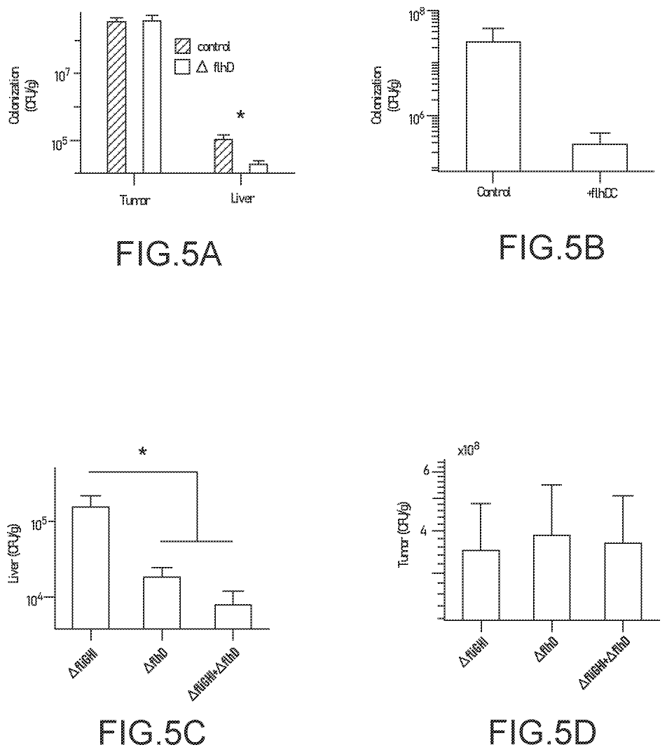

Suppressing flhD expression of Salmonella in systemic circulation improved tumor colonization of the bacteria. While tumor colonization levels were 10⁸ CFU/gram of tumor for both control and ΔflhD Salmonella, liver colonization of ΔflhD Salmonella was reduced ten-fold as compared to control (FIG. 5A; *, P<0.05). When flhDC was overexpressed before injection, however, tumor colonization was impaired compared to a bacterial control (FIG. 5B). These results indicated that flhDC expression before injection increased the clearance rate of Salmonella in the blood. However, suppression of flhDC before injection increased tumor colonization and specificity of Salmonella.

A lack of flhDC activity, not just a lack of flagellar expression, reduced liver colonization while maintaining similar tumor colonization levels of Salmonella. Mice were infected with three different Salmonella strains: ΔflhD, ΔfliGHI, and ΔflhD+ΔfliGHI. The AfiGHI strain lacks flagella but retains flhDC activity. The ΔflhD and ΔflhD+ΔfliGHI, which lack flhDC activity, both colonized livers 8.5 and 20-fold less than the flagellar deficient, ΔfliGHI, strain, respectively (FIG. 5C; *, P<0.05). However, tumor colonization levels of all three strains were not different (FIG. 5D). These results indicated that reduced flhDC activity, and not merely a lack of flagella, increased tumor specificity of Salmonella.

flhDC Expression Increases Intratumoral Dispersion of Salmonella

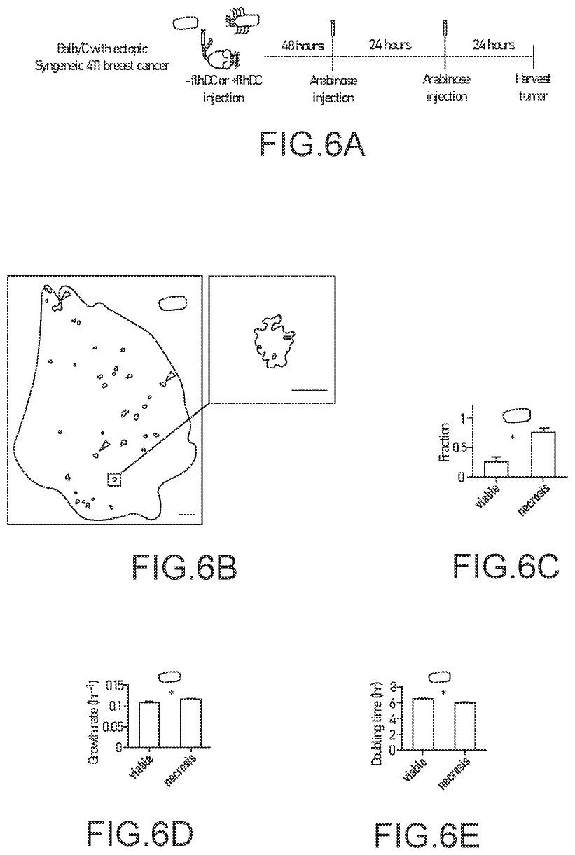

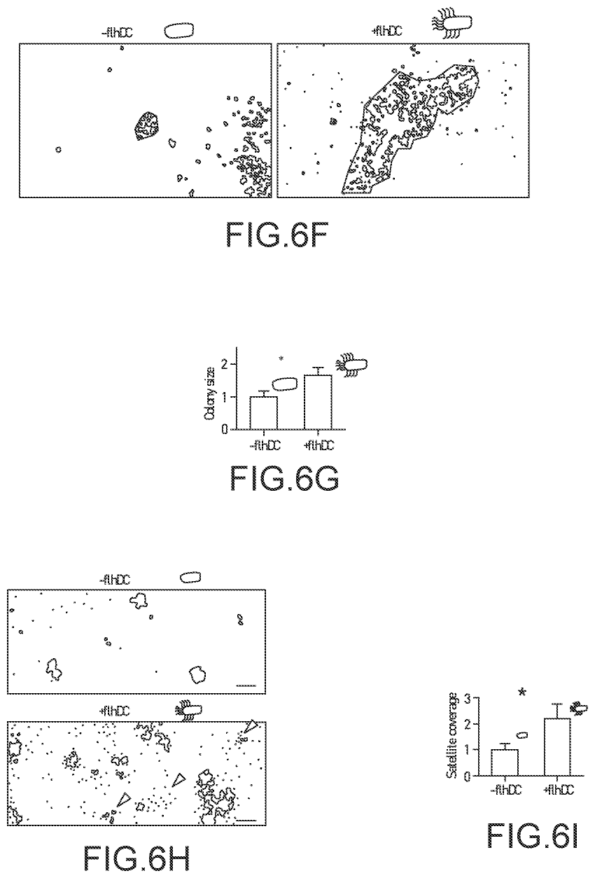

Suppressing expression of flhDC caused Salmonella to predominantly colonize and grow within tumor necrosis. flhDC uninduced were systemically administered into mice and half of the mice were administered arabinose to induce flhDC expression (FIG. 6A). Salmonella not expressing flhDC were not motile and as a result, formed spatially separated, dense colonies predominantly within tumor necrosis (yellow arrow, FIG. 6B). A small fraction of these colonies, however, were located within viable tumor tissue (green arrows, FIG. 26B). The fraction of these dense colonies present in necrosis was 75% percent as compared with 25% percent of colonies in viable tumor tissue (FIG. 6C). If it is assumed that each spatially segregated, dense colony originated from a single bacterium, the growth rate of colonies in necrosis was 0.12 hr-1 as compared to a slightly reduced rate of 0.11 hr-1 within viable tissue (*, P<0.05; FIG. 6D). These bacterial growth rates correspond to doubling times of 6 hours within necrosis and 6.5 within viable tumor tissue (*, P<0.05; FIG. 6E), which is consistent with previous estimates (45). These results indicate that Salmonella heavily favor colonization and growth within tumor necrosis as compared with viable tumor tissue.

Reexpressing flhDC within intratumoral Salmonella increased dispersion and tumor coverage of the bacteria. ΔflhD Salmonella with the PBAD-flhDC genetic circuit were injected intravenously into 4T1 tumor bearing mice and administered two doses of arabinose intraperitoneally to induce flhDC expression in Salmonella (FIG. 6A). flhDC induction of intratumoral Salmonella increased both the bacterial colony size along with bacterial coverage within the tumor (FIG. 6F). The colony size of flhDC reexpressing Salmonella increased 1.5-fold as compared with an uninduced control (*, P<0.05; FIG. 6G). While flhDC uninduced Salmonella formed dense, tightly packed colonies (top panel, FIG. 6H), a larger number of flhDC induced bacteria were located outside of these dense colonies (bottom panel, green arrows, FIG. 6H). The number of Salmonella outside of a dense, central colonies (termed satellite colonies) increased two-fold when flhDC was induced within intratumoral Salmonella (*, P<0.05; FIG. 6I). These results indicate that intratumoral induction of flhDC in Salmonella enables the bacteria to migrate away from dense colonies within tumor necrosis and towards viable cancer cells.

In Situ Expression of flhDC is Needed to Increase Intracellular Invasion of Salmonella into Spatially Distant Cells

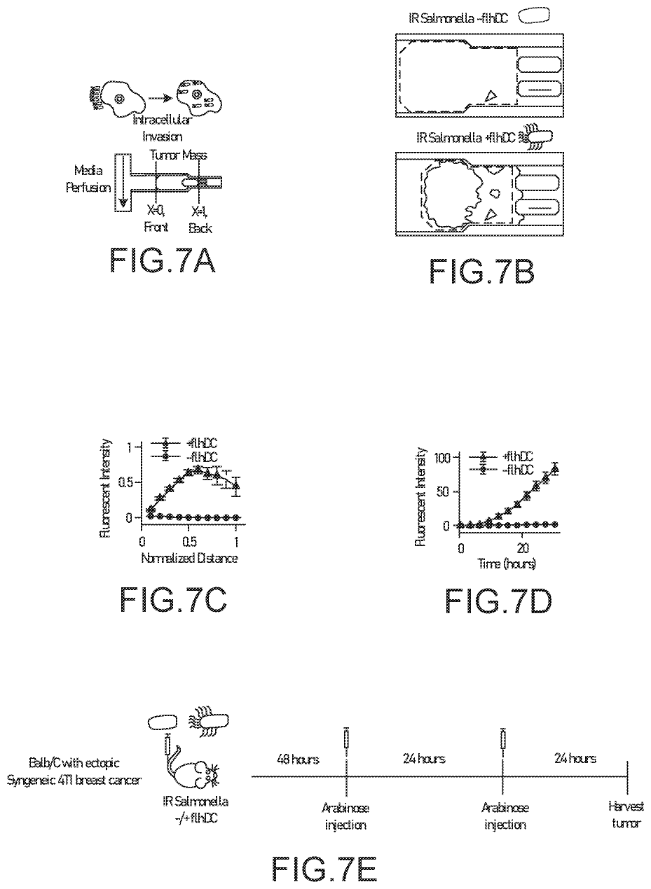

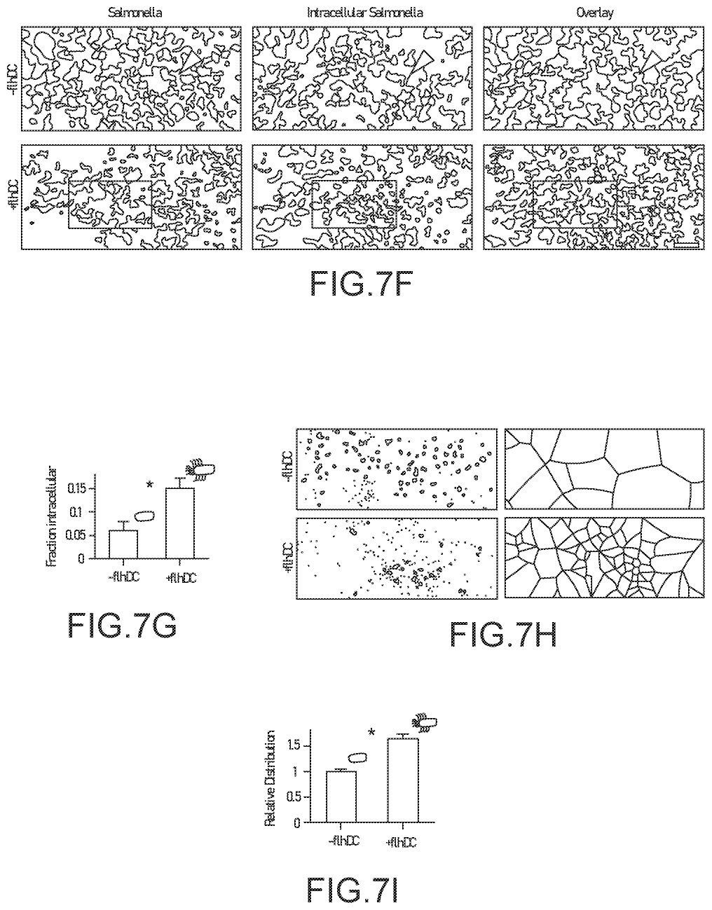

Reexpression of flhDC increased spatial distribution of intracellular Salmonella within tumors. A tumor-on-a-chip device was used to quantify spatial distribution of intracellular Salmonella (FIG. 7A). These Salmonella expressed flhDC with arabinose supplementation and GFP after intracellular invasion (Intracellular reporting Salmonella, IR Sal). Arabinose induction of flhDC enabled broad distribution of intracellular expressing GFP Salmonella within in vitro tumor masses (+flhDC, FIG. 7B). However, uninduced ΔflhD Salmonella (−flhDC) were detected at very low concentrations throughout tumor masses (white arrow, FIG. 7B). The presence of intracellular Salmonella gradually increased deeper into tumor tissue and was enriched 140-fold in +flhDC as compared to −flhDC Salmonella for x>0.5 (**, P<0.01; ***, P<0.001; FIG. 7C). The overall amount of flhDC expressing, IR Salmonella increased exponentially over time as compared to the −flhDC control (*, P<0.05; **, P<0.01; ***, P<0.001; FIG. 7D). This indicated that flhDC induction increased the coverage of intracellular Salmonella in tumor masses.

When +flhDC IR Sal (FIG. 7E) was administered into mice and arabinose induced to express flhDC(FIG. 7F), a greater fraction of bacteria was located intracellularly (Induced, white squares; FIG. 7F). Intracellular invasion of flhDC reexpressing Salmonella increased 2.3-fold over the −flhDC IR Sal control (*, P<0.05; FIG. 7G).

Euclidean distance mapping of histoogical sections, which quantifies the proximity of every location within a tumor to the nearest bacterium, was used to quantify the distribution of intracellular bacteria. The spatial coverage of intracellular bacteria was greater after flhDC induction as indicated by Euclidean distance modeling of histological sections (FIG. 7H). Salmonella were distributed 1.6-fold more after flhDC induction (*, P<0.05; FIG. 7I). These results indicated that flhDC expression increases intracellular invasion by positioning more bacteria near a greater number of viable cancer cells. In addition, flhDC expression increases intracellular invasion in a flagella and T3SS-1 driven manner (10).

Controlling flhDC Expression Improves GFP Delivery Distribution within Tumors

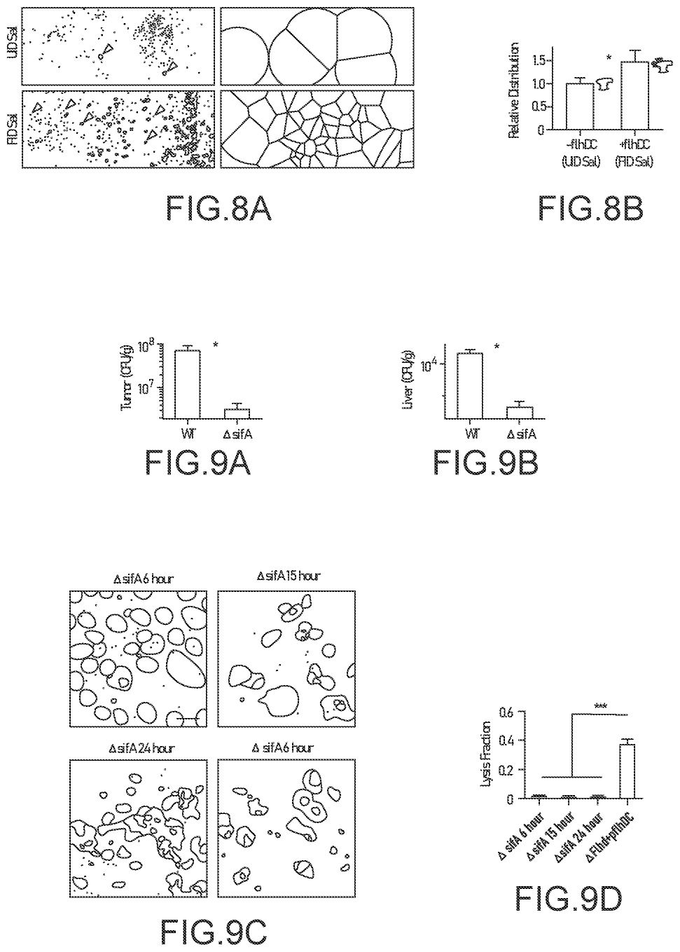

Induction of flhDC within intratumoral engineered Salmonella increased protein delivery over a larger area of cells. Induced Salmonella delivered protein into a broad, spatially distributed set of cells within tumors (FIG. 8A). Euclidean distance mapping analysis of intratumoral delivery demonstrated that flhDC induction (flhDC intracellular delivering Salmonella; FID Sal) increased spatial delivery coverage 1.6-fold as compared to flhDC uninduced (Uninduced intracellular delivering Salmonella; UID Sal) Salmonella (***, P<0.001; FIG. 8B). These results demonstrate that flhDC induction increased spatial coverage in tumors (FIG. 7H, I), which, enabled the bacteria to intracellularly deliver protein into broadly distributed cells within tumors.

### Engineered Salmonella is Superior in Tumor Colonization and Protein Delivery Compared to Exclusively Cytosolic Salmonella

Engineered Salmonella colonized tumors and delivered significantly more protein inside cancer cells compared to conventionally used, cytosolic Salmonella. As demonstrated, ΔflhD Salmonella did not colonize tumors less than a control (FIG. 5A). However, ΔsifA Salmonella colonized tumors ten-fold less than the control (*, P<0.05; FIG. 9A). Liver colonization was also reduced ten-fold between ΔsifA and control Salmonella (*, P<0.05; FIG. 9B) indicating that the ΔsifA strain exhibited overall poor fitness in vivo. Using a selective staining technique to detect bacterial lysis and protein delivery as previously described, engineered Salmonella visibly lysed more than ΔsifA Salmonella inside cells at all time points (FIG. 9C). FID Sal lysed 18-fold more than ΔsifA Salmonella (FIG. 9D; **, P<0.01). Cytosolic localization is important for protein therapies to have biological activity and anti-cancer activity. However, these results demonstrate that predominantly cytosolic Salmonella are not well suited for therapeutic delivery. This is a result of a combination of poor tumor colonization, poor systemic infectivity in vivo and poor lysis efficiency of ΔsifA compared to FID Sal. The ΔsifA strain of Salmonella fails to effectively colonize tumors and therefore, is not advantageous for intracellular protein delivery.

flhDC Expression Reduces Lysis Efficiency within Intracellular Salmonella

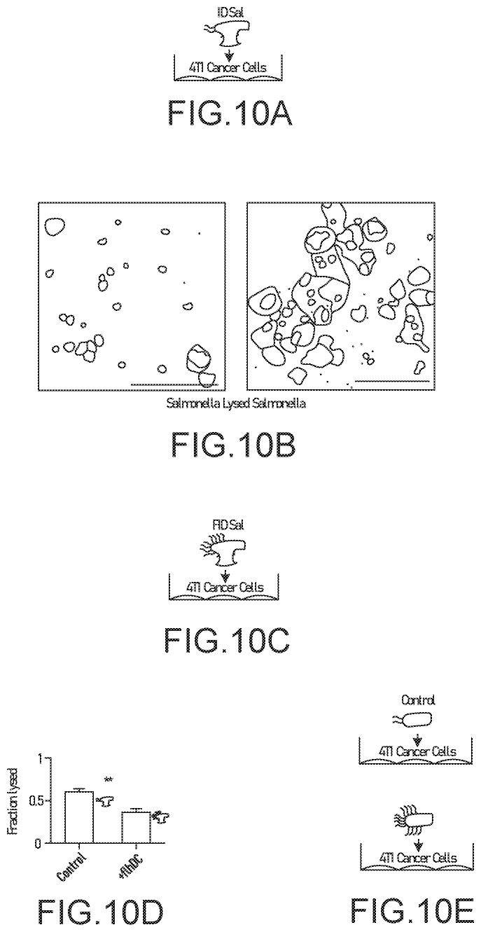

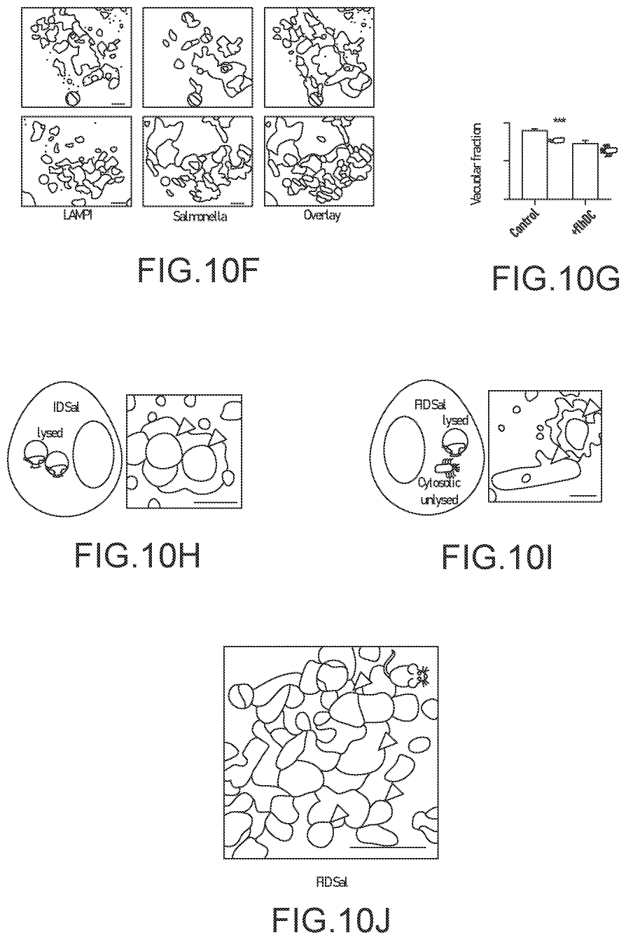

flhDC expression in Salmonella affects intracellular lysis and protein delivery after invasion. To understand this dynamic, cancer cells were infected with control lysing Salmonella (ID Sal) or lysing Salmonella reexpressing flhDC (FID-Sal) (FIG. 10A). As expected, FID Sal invaded cancer cells three times more than ID Salmonella (FIG. 10B, C; **, P<0.01). However, FID Sal lysed 33% less than control ID Sal (FIG. 10D; **, P<0.01). To understand why, the vacuolar/cytosolic distribution of control and flhDC expressing Salmonella was quantified after cancer cell infection (FIG. 10E). While most control Salmonella were contained in vacuoles (colocalized green and red), a larger percentage of flhDC reexpressing Salmonella were cytosolic (green only, FIG. 10F). On a population level, 90% of control were in vacuoles compared to 70% of flhDC reexpressing Salmonella (FIG. 10G). As a result, ID Salmonella were more likely to remain in vacuoles and lyse (white arrows, FIG. 10H) while a small fraction of FID Sal were more likely to escape the vacuole and remain intact (light blue arrows, FIG. 10I). In vivo, FID Sal qualitatively demonstrated a similar phenomenon (FIG. 10J). Unlysed and intracellular FID Sal were distributed throughout several cells (white arrows), likely, indicating that the bacteria were hyper-replicating in the cytoplasm of the tumor cells. These results indicate that flhDC induction increases invasion but decreases lysis efficiency of engineered Salmonella, likely because of vacuolar escape.

Vacuolar Retention of flhDC Overexpressing Salmonella Rescues Lysis and Protein Delivery Efficiency

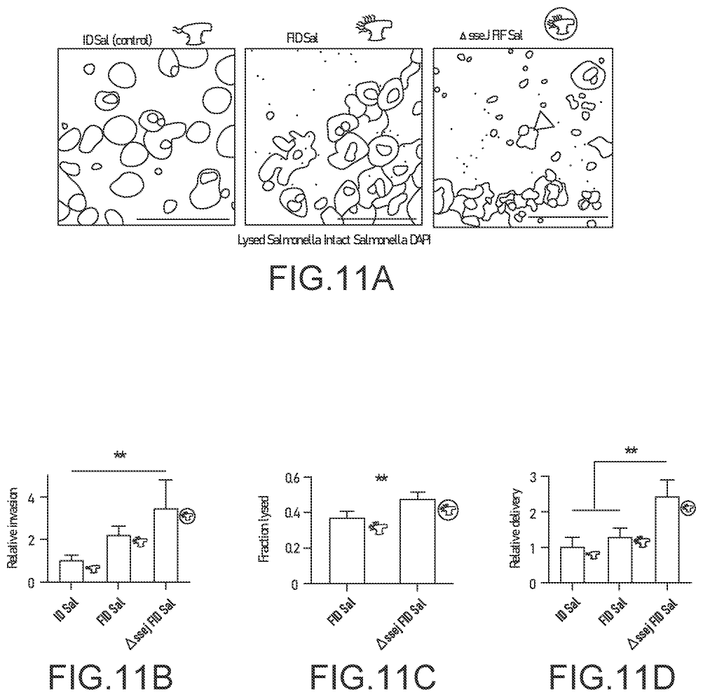

Overexpressing flhDC in a vacuole escape impaired strain of engineered Salmonella rescued lysis efficiency and overall intracellular protein delivery. It was previously demonstrated that engineered ΔsseJ Salmonella intracellularly lysed with high efficiency. It was therefore hypothesized that overexpressing flhDC in lysing ΔsseJ Salmonella (ΔsseJ FID Sal) would rescue lysis efficiency while maintaining high levels of invasion. Cells infected with ΔsseJ FID Sal exhibited an increase in invaded, lysed bacteria (white arrow, FIG. 11A). The ΔsseJ FID Sal invaded cancer cells 1.5-fold more than FID Sal and three-fold more than ID Sal (FIG. 11B, **, P<0.01). Intracellular ΔsseJ FID Sal also lysed 25% more efficiently than FID Sal alone (FIG. 11C; **, P<0.01). The combination of these two phenomena (increased invasion and improved lysis) of the engineered strain increased overall protein delivery 2.5-fold over FID Sal (FIG. 11D; **, P<0.01). This data demonstrated that the reduced lysis efficiency resulting from flhDC activity could be rescued by overexpressing the transcription factor in Salmonella engineered to remain in vacuoles.

### Conclusions

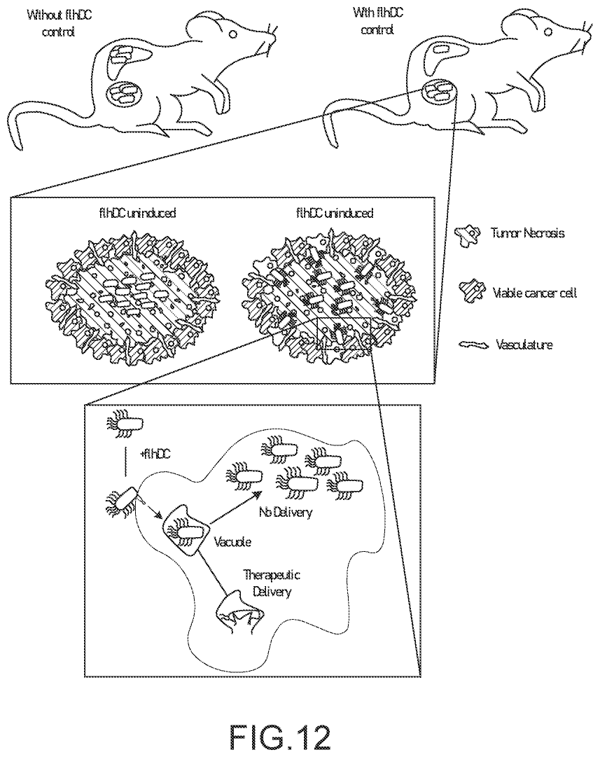

Modulating flhDC expression in engineered Salmonella had broad implications for intracellular therapeutic delivery within tumors (FIG. 12). Salmonella devoid of flhDC expression colonized tumors more selectively. However, overexpression of the transcription factor within systemic Salmonella decreased tumor colonization of the bacteria. Controlled expression of flhDC in tumors increased spatial distribution of extracellular and intracellular Salmonella. While flhDC expression reduced intracellular lysis efficiency of engineered Salmonella, overexpressing the transcription factor in a vacuolar resident, ΔsseJ, strain rescued lysis efficiency and improved overall protein delivery in tumor cells. Together, results demonstrate the modulating flhDC expression in therapeutic Salmonella improves several driving features of protein delivery in tumors (FIG. 12).

### Discussion

It is shown herein that controlling flhDC expression of engineered bacteria maintains high colonization levels, improves tumor specificity and increases protein delivery distribution within tumors. Expression of flhDC also decreased intracellular lysis efficiency but was rescued by overexpressing the transcription factor in a vacuole localized strain (ΔsseJ) of Salmonella. The combination of the two genetic engineering strategies increased overall intracellular protein delivery.

The colonization pattern of flhDC uninduced Salmonella suggests that only a few hundred single bacteria infiltrate tumors and grow in situ out of the two million that are injected. These ratios are corroborated by earlier work demonstrating that one out of ten thousand bacteria adhere to tumor vasculature (46). In histological samples flhDC uninduced Salmonella form spatially separated colonies overwhelmingly localized to tumor necrosis (FIG. 6B, C). Each of these colonies could originate from clonal expansion of a single bacteria that managed to colonize the tumor. If this is the case, it would suggest that bacterial influx into tumors occurs as a rare event, is strongly assisted by extensive necrosis, and is the rate limiting step of tumor colonization. Such a rare bacterial infiltration event could explain why tumor colonization is highly variable within populations of mice or humans as described previously (47). These results could explain why extensive tumor colonization was predominantly detected predominantly in the presence of tumor necrosis in humans (47). Combining tumor vascular disrupting agents with Salmonella could therefore reduce treatment variability between patients and enable effective colonization of small, necrosis deficient primary and metastatic tumors.

Two strategies could be used to robustly initiate bacterial colonization within tumors: (1) Co-administering bacteria along with a mild TNF-alpha inducer as previously described (48) or (2) genetically modifying Salmonella to evade systemic innate immune recognition (e.g., flhDC modulation). In scenario (1) as previously demonstrated, administration with lipid A (a known TNF-alpha inducing agent) did not cause septic shock but increased vascular permeability and therefore, could have increased the probability of bacterial infiltration into tumors across a large number of mice. In scenario (2), flhDC suppression of injected Salmonella could help the bacteria evade innate immune detection of flagella in systemically circulating bacteria. This could enable bacteria to persist longer systemically without causing septic shock. Longer systemic persistence could, in turn, increase the probability of bacterial infiltration into tumors.

Wild type Salmonella are likely not optimized to deliver therapies intracellularly within tumors. One reason for this might be that necrotic tumor tissue facilitates cecile and non-motile colonization of Salmonella. The data suggests that tumors select for non-motile and likely, non-flagellated bacteria since flagellated bacteria minimally colonize tumors (FIG. 5B) likely due to innate immune mediated clearance (8, 9). The flhDC uninduced bacteria were not impaired in colonization levels as compared to the control strain (FIG. 5D). Moreover, flhDC uninduced bacteria clustered in densely packed colonies largely located within tumor necrosis (FIG. 6B). This suggests that Salmonella have a higher affinity to colonize necrosis rather than viable tissue and that external control is required to enable Salmonella to invade viable tumors cells in an flhDC dependent manner. By controllably activating flhDC expression in intratumoral Salmonella, it was demonstrated that a significant fraction of these bacteria invaded and delivered protein into a spatially distributed set of cells.

Vacuolar residence could also aid in preventing premature clearance before tumor accumulation in addition to enabling lysis of engineered Salmonella. The current paradigm for intracellular, cytosolic therapeutic delivery is to enable Salmonella to escape the vacuole and directly invade the cytosol through deletion of the sifA gene (20). Similarly, bacterial variants expressing listeriolysin O have also been used to enable vacuolar escape of therapeutic Salmonella (49-51). However, it was determined that unnatural cytosolic escape of Salmonella (ΔsifA) reduced tumor colonization 100-fold compared to the parental strain (FIG. 9A). This is likely because cytosolic pathogens elicit a strong antimicrobial and NF-kB dependent immune response that is detrimental to bacterial fitness in vivo (21, 52-55). The ΔsifA bacteria also lysed 18-fold less than FID Salmonella. These results indicate that the engineered strain significantly improved the delivery potential Salmonella as compared to existing cytosolic delivery methods.

The engineered bacterial system described herein shares similarities with strains of Salmonella Typhi that have evolved to systemically infect human hosts. Humans serve as the natural host for Salmonella Typhi and upon ingestion, the bacteria stealthily translocate from the gut into systemic circulation without attracting a significant initial immune response (30). The bacteria can circulate systemically for extended periods of time without causing septic shock (30). The typhoidal strain accomplishes this by encoding a capsular regulatory protein, TviA. The transcription factor encodes for the Vi capsule that masks bacterial LPS (56). In addition, TviA suppresses flagellar and T3SS-1 activity in systemically circulating bacteria through repression of flhDC and HiLA expression, respectively 57. Masking of the LPS and downregulation of flagellar and T3SS-I activity leads to evasion of innate immune recognition (57). The instant delivery strain of Salmonella also has a modified LPS through deletion of msbB which prevents sepsis. In addition, the expression of flhDC, which activates flagellar and to a lesser extent, T3SS-1 synthesis (10), is suppressed upon systemic administration of the engineered Salmonella. The engineered strain of Salmonella and Salmonella Typhi also share the similarity that both types of bacteria reside mostly within the intracellular vacuole. Residence within the intracellular vacuole prevents bacterial detection by cytosolic, innate immune sensors like nod-like receptors, ubiquitin and NF-kB components. These genetic modifications likely act to mask common pathogen associated molecular patterns associated with Salmonella and increase systemic persistence without causing any adverse immune responses.

### BIBLIOGRAPHY

1. Uhlen, M., et al., Proteomics. Tissue-based map of the human proteome. Science, 2015. 347(6220): p. 1260419.
2. Au, J. L., et al., Delivery of cancer therapeutics to extracellular and intracellular targets: Determinants, barriers, challenges and opportunities. Adv Drug Deliv Rev, 2016. 97: p. 280-301.
3. Gauger, E. J., et al., Role of motility and the flhDC Operon in Escherichia coli MG1655 colonization of the mouse intestine. Infect Immun, 2007. 75(7): p. 3315-24.
4. Wang, X. and T. K. Wood, IS5 inserts upstream of the master motility operon flhDC in a quasi-Lamarckian way. ISME J, 2011. 5(9): p. 1517-25.
5. Macnab, R. M., Genetics and biogenesis of bacterial flagella. Annu Rev Genet, 1992. 26: p. 131-58.
6. Winter, S. E., et al., The TviA auxiliary protein renders the Salmonella enterica serotype Typhi RcsB regulon responsive to changes in osmolarity. Mol Microbiol, 2009. 74(1): p. 175-193.
7. Winter, S. E., et al., The Salmonella enterica serotype Typhi regulator TviA reduces interleukin-8 production in intestinal epithelial cells by repressing flagellin secretion. Cell Microbiol, 2008. 10(1): p. 247-61.
8. Yoon, S. I., et al., Structural basis of TLR5-flagellin recognition and signaling. Science, 2012. 335(6070): p. 859-64.
9. Franchi, L., et al., Cytosolic flagellin requires Ipaf for activation of caspase-1 and interleukin 1beta in salmonella-infected macrophages. Nat Immunol, 2006. 7(6): p. 576-82.
10. Raman, V., et al., The motility regulator flhDC drives intracellular accumulation and tumor colonization of Salmonella. J Immunother Cancer, 2019. 7(1): p. 44.
11. Benoun, J. M., et al., Optimal protection against Salmonella infection requires noncirculating memory. Proc Natl Acad Sci USA, 2018. 115(41): p. 10416-10421.
12. Finn, C. E., et al., A second wave of Salmonella T3SS1 activity prolongs the lifespan of infected epithelial cells. PLoS Pathog, 2017. 13(4): p. e1006354.
13. Singer, H. M., M. Erhardt, and K. T. Hughes, RflM functions as a transcriptional repressor in the autogenous control of the Salmonella Flagellar master operon flhDC. J Bacteriol, 2013. 195(18): p. 4274-82.
14. Zeng, H., et al., Flagellin is the major proinflammatory determinant of enteropathogenic Salmonella. J Immunol, 2003. 171(7): p. 3668-74.
15. Stecher, B., et al., Flagella and chemotaxis are required for efficient induction of Salmonella enterica serovar Typhimurium colitis in streptomycin-pretreated mice. Infect Immun, 2004. 72(7): p. 4138-50.
16. Li, Z., et al., Pyroptosis of Salmonella Typhimurium-infected macrophages was suppressed and elimination of intracellular bacteria from macrophages was promoted by blocking QseC. Sci Rep, 2016. 6: p. 37447.
17. Stritzker, J., et al., Enterobacterial tumor colonization in mice depends on bacterial metabolism and macrophages but is independent of chemotaxis and motility. Int J Med Microbiol, 2010. 300(7): p. 449-56.
18. Yang, N., et al., Attenuated Salmonella typhimurium carrying shRNA-expressing vectors elicit RNA interference in murine bladder tumors. Acta Pharmacol Sin, 2011. 32(3): p. 368-74.
19. Guo, H., et al., Targeting tumor gene by shRNA-expressing Salmonella-mediated RNAi.

Gene Ther, 2011. 18(1): p. 95-105.
20. Camacho, E. M., et al., Engineering Salmonella as intracellular factory for effective killing of tumour cells. Sci Rep, 2016. 6: p. 30591.
21. van Wijk, S. J. L., et al., Linear ubiquitination of cytosolic Salmonella Typhimurium activates NF-kappaB and restricts bacterial proliferation. Nat Microbiol, 2017. 2: p. 17066.
22. Bierschenk, D., et al., The Salmonella pathogenicity island-2 subverts human NLRP3 and NLRC4 inflammasome responses. J Leukoc Biol, 2019. 105(2): p. 401-410.
23. Steele-Mortimer, O., The Salmonella-containing vacuole: moving with the times. Curr Opin Microbiol, 2008. 11(1): p. 38-45.
24. Liss, V., et al., Salmonella enterica Remodels the Host Cell Endosomal System for Efficient Intravacuolar Nutrition. Cell Host Microbe, 2017. 21(3): p. 390-402.
25. Chakravortty, D., I. Hansen-Wester, and M. Hensel, Salmonella pathogenicity island 2 mediates protection of intracellular Salmonella from reactive nitrogen intermediates. J Exp Med, 2002. 195(9): p. 1155-66.
26. Eswarappa, S. M., et al., Division of the Salmonella-containing vacuole and depletion of acidic lysosomes in Salmonella-infected host cells are novel strategies of Salmonella enterica to avoid lysosomes. Infect Immun, 2010. 78(1): p. 68-79.
27. Ruiz-Albert, J., et al., Complementary activities of SseJ and SifA regulate dynamics of the Salmonella typhimurium vacuolar membrane. Mol Microbiol, 2002. 44(3): p. 645-61.
28. Hallstrom, K. and B. A. McCormick, Salmonella Interaction with and Passage through the Intestinal Mucosa: Through the Lens of the Organism. Front Microbiol, 2011. 2: p. 88.
29. Parkhill, J., et al., Complete genome sequence of a multiple drug resistant Salmonella enterica serovar Typhi CT18. Nature, 2001. 413(6858): p. 848-52.
30. Dougan, G. and S. Baker, Salmonella enterica serovar Typhi and the pathogenesis of typhoid fever. Annu Rev Microbiol, 2014. 68: p. 317-36.
31. Hodak, H. and J. E. Galan, A Salmonella Typhi homologue of bacteriophage muramidases controls typhoid toxin secretion. EMBO Rep, 2013. 14(1): p. 95-102.
32. Spano, S., J. E. Ugalde, and J. E. Galan, Delivery of a Salmonella Typhi exotoxin from a host intracellular compartment. Cell Host Microbe, 2008. 3(1): p. 30-8.
33. Choi, J., et al., <em>Salmonella</em> pathogenicity island 2 expression negatively controlled by EIIA<sup>Ntr</sup>-SsrB interaction is required for <em>Salmonella</em>virulence. Proceedings of the National Academy of Sciences, 2010. 107(47): p. 20506-20511.
34. Zhang, K., et al., Minimal SPI1-T3SS effector requirement for Salmonella enterocyte invasion and intracellular proliferation in vivo. PLoS Pathog, 2018. 14(3): p. e1006925.
35. Smith, C., et al., Mapping the Regulatory Network for Salmonella enterica Serovar Typhimurium Invasion. MBio, 2016. 7(5).
36. Brawn, L. C., R. D. Hayward, and V. Koronakis, Salmonella SPI1 effector SipA persists after entry and cooperates with a SPI2 effector to regulate phagosome maturation and intracellular replication. Cell Host Microbe, 2007. 1(1): p. 63-75.
37. Xu, Y., et al., A Bacterial Effector Reveals the V-ATPase-ATG16L1 Axis that Initiates Xenophagy. Cell, 2019. 178(3): p. 552-566 e20.
38. Chong, A., et al., A role for the Salmonella Type III Secretion System 1 in bacterial adaptation to the cytosol of epithelial cells. Mol Microbiol, 2019. 112(4): p. 1270-1283.
39. Ilyas, B., et al., Regulatory Evolution Drives Evasion of Host Inflammasomes by Salmonella Typhimurium. Cell Rep, 2018. 25(4): p. 825-832 e5.
40. Swofford, C. A., N. Van Dessel, and N. S. Forbes, Quorum-sensing Salmonella selectively trigger protein expression within tumors. Proc Natl Acad Sci USA, 2015. 112(11): p. 3457-62.
41. Mosberg, J. A., et al., Improving lambda red genome engineering in Escherichia coli via rational removal of endogenous nucleases. PLoS One, 2012. 7(9): p. e44638.
42. Mosberg, J. A., M. J. Lajoie, and G. M. Church, Lambda red recombineering in Escherichia coli occurs through a fully single-stranded intermediate. Genetics, 2010. 186(3): p. 791-9.
43. Toley, B. J. and N. S. Forbes, Motility is critical for effective distribution and accumulation of bacteria in tumor tissue. Integr Biol, 2012. 4(2): p. 165-76.
44. Walsh, C. L., et al., A multipurpose microfluidic device designed to mimic microenvironment gradients and develop targeted cancer therapeutics. Lab on a chip, 2009. 9: p. 545-54.
45. Leschner, S., et al., Tumor invasion of Salmonella enterica serovar Typhimurium is accompanied by strong hemorrhage promoted by TNF-alpha. PLoS One, 2009. 4(8): p. e6692.
46. Forbes, N. S., et al., Sparse initial entrapment of systemically injected Salmonella typhimurium leads to heterogeneous accumulation within tumors. Cancer Res, 2003. 63(17): p. 5188-93.
47. Toso, J. F., et al., Phase I study of the intravenous administration of attenuated Salmonella typhimurium to patients with metastatic melanoma. J Clin Oncol, 2002. 20(1): p. 142-52.
48. Zhang, M. and N. S. Forbes, Trg-deficient Salmonella colonize quiescent tumor regions by exclusively penetrating or proliferating. J Control Release, 2015. 199: p. 180-9.
49. Critchley, R. J., et al., Potential therapeutic applications of recombinant, invasive E. coli.

Gene Ther, 2004. 11(15): p. 1224-33.
50. Critchley-Thorne, R. J., A. J. Stagg, and G. Vassaux, Recombinant Escherichia coli expressing invasin targets the Peyer's patches: the basis for a bacterial formulation for oral vaccination. Mol Ther, 2006. 14(2): p. 183-91.
51. Higgins, D. E., N. Shastri, and D. A. Portnoy, Delivery of protein to the cytosol of macrophages using Escherichia coli K-12. Mol Microbiol, 1999. 31(6): p. 1631-41.
52. Nelson, R. H. and D. E. Nelson, Signal Distortion: How Intracellular Pathogens Alter Host Cell Fate by Modulating NF-kappaB Dynamics. Front Immunol, 2018. 9: p. 2962.
53. Rahman, M. M. and G. McFadden, Modulation of NF-kappaB signalling by microbial pathogens. Nat Rev Microbiol, 2011. 9(4): p. 291-306.
54. Gaudet, R. G., et al., Innate Recognition of Intracellular Bacterial Growth Is Driven by the TIFA-Dependent Cytosolic Surveillance Pathway. Cell Rep, 2017. 19(7): p. 1418-1430.
55. Liu, T., et al., NF-kappaB signaling in inflammation. Signal Transduct Target Ther, 2017. 2.
56. Hu, X., et al., Vi capsular polysaccharide: Synthesis, virulence, and application. Crit Rev Microbiol, 2017. 43(4): p. 440-452.
57. Winter, S. E., et al., Salmonella enterica Serovar Typhi conceals the invasion-associated type three secretion system from the innate immune system by gene regulation. PLoS Pathog, 2014. 10(7): p. e1004207.

### Example III

Chromosomal Integration of flhD in EBV-002.

The cell invasive capability of EBV-002 containing a single, chromosomal copy of PBAD-flhDC was assessed. Chromosomal integration of an inducible version of flhDC can create a master delivery vehicle that could be used to deliver any therapy into a tumor. Creating a single master delivery vehicle can streamline the manufacturing process of any EBV based therapy. To this end, a single copy of PBAD-flhDC was integrated in place of the endogenous flhDC gene within VNP20009 Salmonella. This chromosomally integrated strain was grown with arabinose to activate flhDC expression and used to infect cancer cells. The chromosomally integrated VNP20009 invaded cancer cells to similar levels as the bacteria containing episomal copies of flhDC (FIG. 13A). The chromosomal knockin of flhDC also was similarly inducible as compared to Salmonella with episomal PBAD-flhDC (FIG. 13B). This result indicated that the flhDC inducible genetic circuit could be genomically integrated in order to create a master EBV-002 delivery vehicle.

### Development of a Clinical Strain of EBV-002.

A clinically compatible strain of EBV-002 was created by controlling activation of flhDC with salicylic acid, the active ingredient in aspirin (FIG. 14). Since flhDC is the main transcription factor controlling flagellar synthesis, chemotaxis and motility, bacteria are highly sensitive to even low expression levels of the protein. As a result, it was hypothesized that expression levels in the uninduced state would need to be tightly repressed in order to fully suppress uninduced cell invasion. To test this hypothesis, four different flhDC inducible EBV-002 strains were produced: salicylic induced 1) flhD, 2) flhD containing a weakly active ssrA degradation sequence, 3) flhD containing a moderately active ssrA degradation sequence and 4) flhD containing a highly active degradation tag (FIG. 14). The purpose of these degradation tags was to eliminate uninduced flhD activity that was a result of leaky expression from the pSal promoter. The intracellular invasion rates of each of these four strains were compared to the PBAD inducible version of flhDC. As expected, sample (1) was highly motile and invasive, with or without salicylic acid induction (FIG. 14B) indicating that the salicylic acid promoter was leaky. Samples (2), (3) and (4) only invaded cells after salicylic acid induction and were completely non-invasive otherwise. However, samples (2) and (3) were the most intracellularly invasive after aspirin induction (FIG. 14B). Most importantly, strains (2) and (3) were more invasive as compared to the PBAD inducible version of EBV-002 (FIG. 14B). These results demonstrate that the salicylic acid induction circuit was optimized to express flhD and regulate intracellular invasion of EBV-002 into cancer cells.

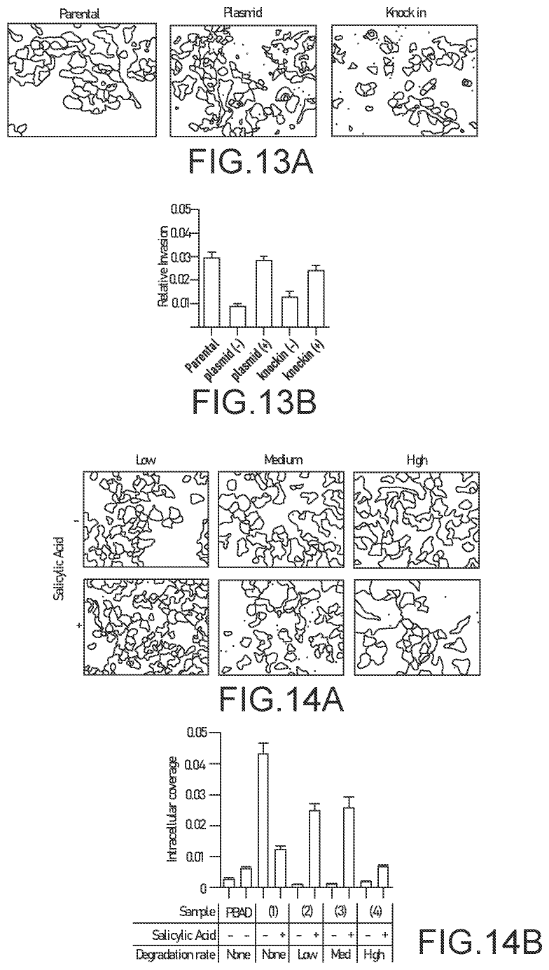

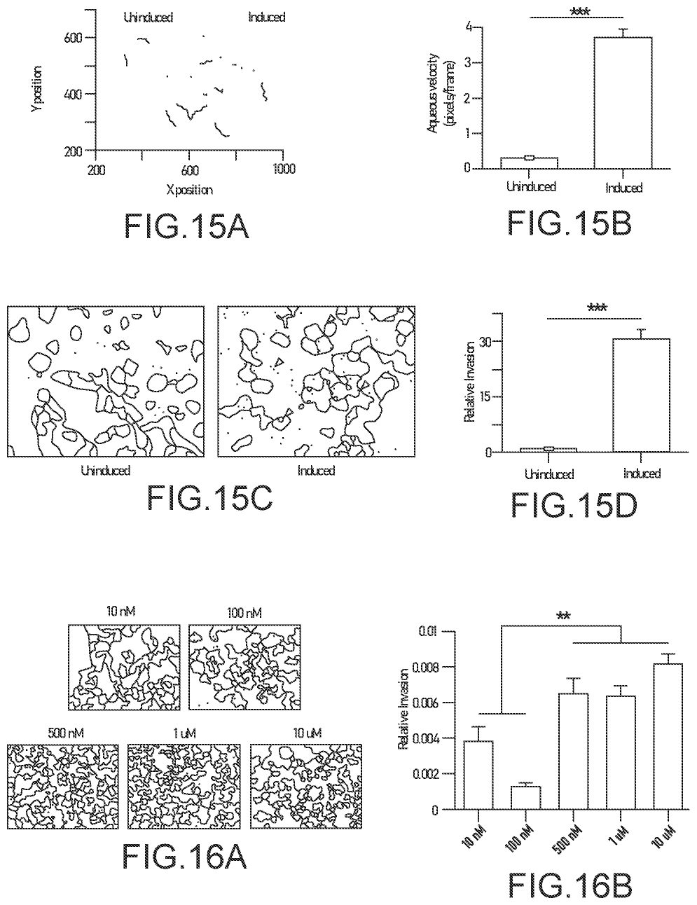

Sample (2) was characterized since this strain of EBV-002 had the highest range of activation between uninduced and induced samples. The induced bacteria swam a significantly longer distance as compared to uninduced EBV-002, which, remained stationary (FIG. 15A). Salicylate induced EBV-002 swam 12.7-fold farther than the uninduced control (***, P<0.001; FIG. 15B). This indicated that the salicylic acid inducible genetic circuit could robustly control flhDC activity in the clinical EBV-002 strain. As expected, the salicylic acid induced, clinical strain of EBV-002 invaded cancer cells 30 times more than the uninduced control (***, P<0.001; FIG. 15C, D). These results indicate that expressing flhD with a weakly active degradation tag using salicylic acid enabled the greatest control of intracellular invasion of EBV-002.

After determining which version of pSal-flhD was most effective at invading cancer cells with salicylic acid induction, the lowest amount of salicylic acid needed to enable intracellular invasion was determined next. EBV-002 was induced with either 10 nanomolar (nM), 100 nM, 500 nM, 1 micromolar (uM) or 10 uM salicylic acid and infected cancer cells with each of these strains. It was determined that a 500 nM concentration of salicylic acid was needed to enable intracellular invasion of EBV-002 (FIG. 16). This result is significant because it indicates that the induction threshold for EBV-002 is well within the concentration range of salicylic acid found in the blood stream (10-50 uM) after a person orally ingests aspirin. Together, these results indicate that EBV-002 is ready for use as an intracellular delivery vehicle within human tumors. Incorporation of the ΔsseJ mutation into EBV-002 to create EBV-003.

The ΔsseJ mutation was previously demonstrated to significantly increased lysis efficiency of the EBV strain. To this end, the EBV-002 strain containing the same salicylic acid inducible flhDC gene as well as the intracellular lysis cassette was additionally engineered with the ΔsseJ mutation in order to create EBV-003.

### In Vivo Efficacy of EBV-003.

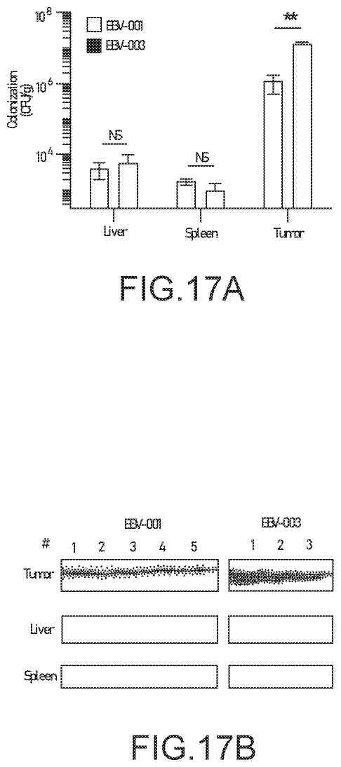

Biodistribution and tumor selective protein delivery were assessed in mice bearing subcutaneous 4T1 tumors. Balb/C mice with ˜750 mm³ subcutaneous tumors were intravenously injected with 1×10⁷ CFU of EBV-003. At 72 hours p.i., mice were intraperitoneally injected with 5 mg of salicylic acid to induce flhDC expression within intratumoral bacteria. 24 hours later, mice were sacrificed and tumors, livers, and spleens were excised for analysis. Colonization and protein delivery of EBV-003 was compared to EBV-001 to assess any improvements. After colonization, EBV-003 colonized tumors 10.7-fold more than EBV-001 while keeping spleen and liver colonization unchanged (FIG. 17A, **, P<0.01). On average, EBV-003 delivered 31-fold more protein into tumor cells as compared to EBV-001 (FIG. 17B). Protein delivery was not, however, detected in the spleen or livers with either strain. These results demonstrate that EBV-003 is significantly more effective at colonizing and delivering protein selectively into tumors while sparing healthy tissue.

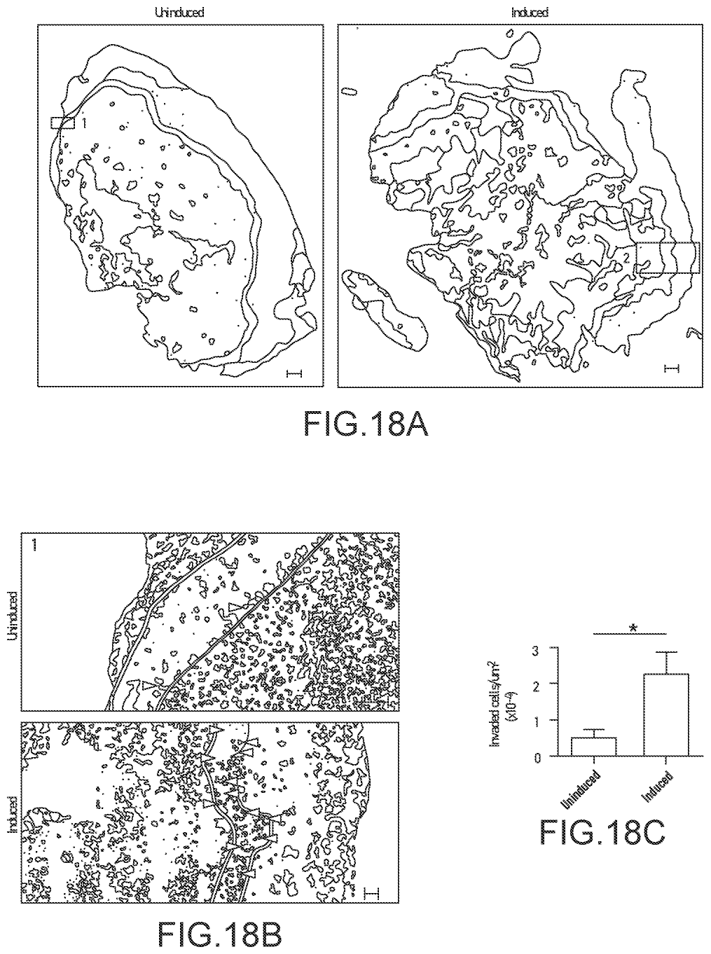

To determine whether EBV-003 intracellularly invaded cancer cells after salicylic acid induction in vivo, female balb/c mice were subcutaneously injected with 4T1 tumors. Once tumors were 500 mm³, the mice were injected with 1×10⁶ CFUs via the tail vein. Seventy-two hours after bacterial administration, seven of the mice were intraperitoneally injected with 5 mg of sodium salicylate while four were given a saline injection as a control. Twenty-four hours after salicylic acid administration, the mice were sacrificed, tumors were excised, fixed and stained for Salmonella. Histological examination revealed that salicylic acid induction increased intracellular invasion of viable cancer cells within quiescent tumor tissue. More bacteria (Red Xs, FIG. 18A) were distributed across the quiescent tumor tissue after induction with salicylic acid (FIG. 18B). Salicylic acid induction resulted in a two-fold increase in cancer cells with intracellular EBV-003 as compared to the uninduced control (*, P<0.05; FIG. 18C). These results indicated that EBV-003 could be induced to invade cells using a therapeutic dose of salicylic acid.

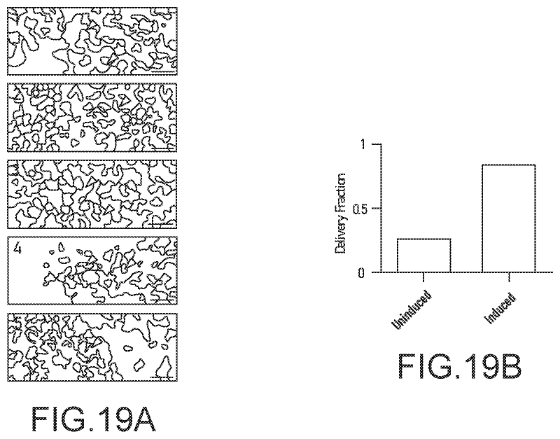

Intracellular protein delivery with EBV-003 was also evaluated with and without salicylate induction. After salicylic acid induction, protein delivery was detected in five out of six tumors within the transition zones where tumor cells are rapidly dividing (white arrows, FIG. 19A). Whereas, delivery was only detected within the transition zone in one of the four uninduced, control mice (FIG. 19B). These results demonstrated that salicylate induction of EBV-003 enabled intracellular protein delivery in vivo.

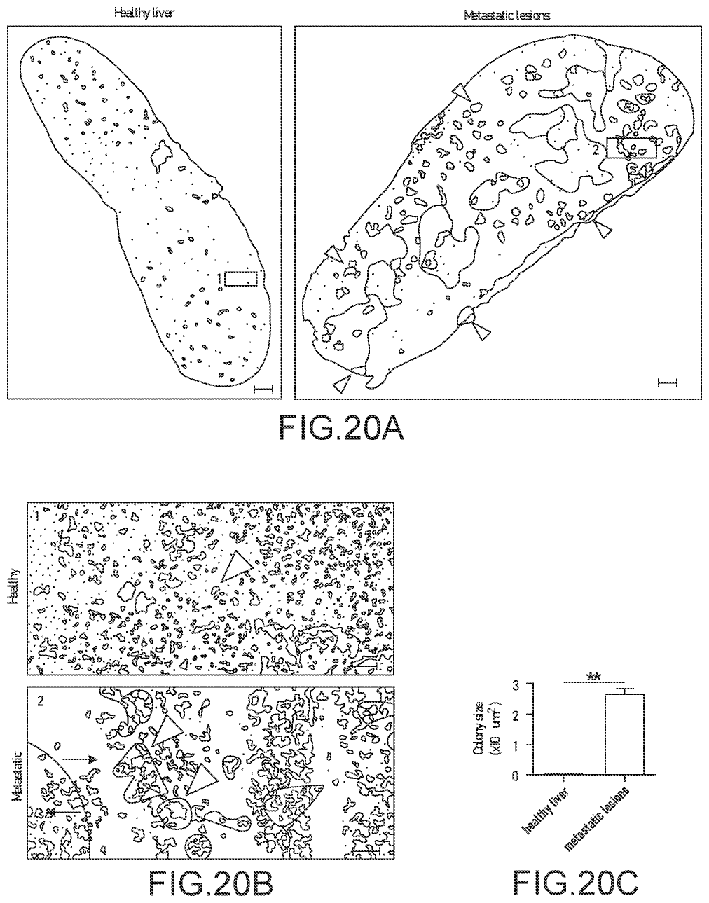

In vivo colonization, invasion and protein delivery of EBV-003 in spontaneous breast cancer metastasis in the liver. The EBV-003 strain colonized, invaded and delivered protein selectively into metastatic breast cancer within the liver (FIG. 20). All dense bacterial colonies were only found within the metastatic breast cancer lesions within the liver (white outlined colonies, FIG. 20A). Moreover, 85% of these colonies were immediately adjacent, or within actively dividing tumor lesions (red arrows, FIG. 20A), where therapeutic delivery is most effective. On the other hand, colonies found in healthy tissue were observed far less frequently and were much smaller in size (FIG. 20A). Bacterial colonies were rarely spotted in healthy tissue and were very small (1, white arrow, FIG. 20B). However, in the metastatic lesions, the colonies appeared significantly larger in area (2, white arrows, FIG. 20B). Within the liver, 87.7% of colonies were found within the metastatic lesions while the other 12.3% were found within healthy liver tissue. Moreover, the size of the colonies within the metastatic lesions was over 118 times greater than the size of colonies in healthy tissue (***, p=2.2×10-26; FIG. 20C). This equates to an 850-fold enrichment of EBV-003 in metastatic breast cancer lesions within the liver versus the immediately adjacent healthy tissue. While we have demonstrated the ability of therapeutic Salmonella to colonize primary tumors greater than 1,000-fold more than any other organ, this is the first demonstration that Salmonella preferentially colonize metastatic tumor lesions as compared to immediately adjacent healthy tissue to a similarly high magnitude. This illustrates the exquisite selectivity of EBV-003 to colonize tumor tissue regardless of whether the tumors are primary or metastatic lesions.

The EBV-003 strain also intracellularly invaded cancer cells within liver metastases (white arrows, FIG. 21A). However, there was no difference in invasion levels between salicylate induced and uninduced EBV-003 (FIG. 21B). One reason for this could be that most of the metastatic lesions contained a higher fraction of viable tumor tissue and lower amount of necrosis. As a result, EBV-003 bacteria were more likely to be in close proximity to viable tumor cells increasing the likelihood that the bacteria could intracellularly invade the cells regardless of induction status. This is in contrast to primary tumor tissue, where salicylate induction of flhDC increased the intracellular presence of EBV-003 within the quiescent tumor tissue (FIG. 18A). This could be because the bacteria preferentially colonized necrosis and required flhDC dependent motility to swim towards and intracellularly invade the actively dividing cancer cells. Therefore, this indicates that flhDC induction is necessary for intracellular invasion within a primary tumor mass but less so within small, non-necrotic metastatic or primary lesions.

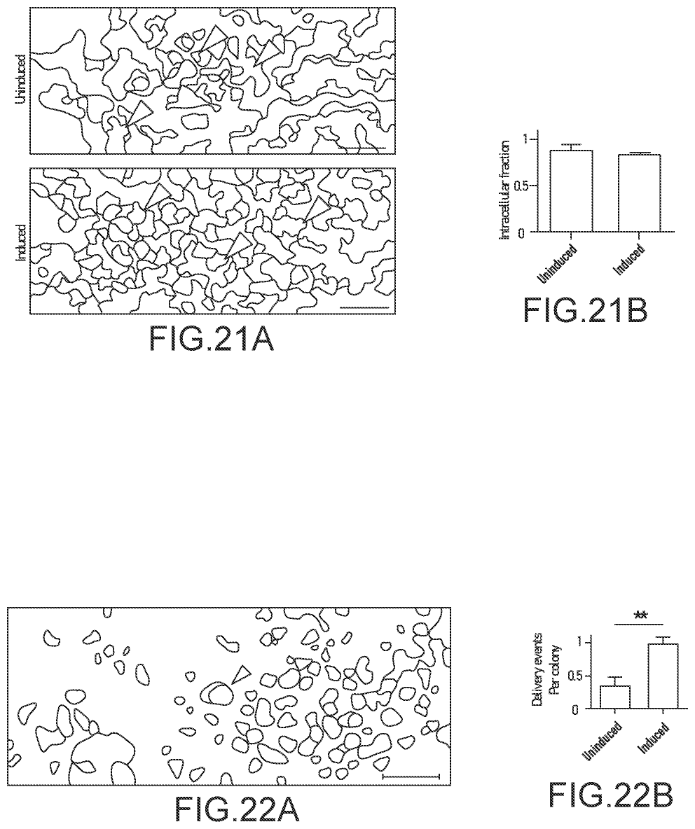

Although EBV-003 seemed to invade metastatic cancer cells in the presence or absence of flhDC activity, protein delivery was detected at higher frequencies with salicylate induction in vivo. Cytosolic delivery into cells within metastatic tumors was detected histologically (white arrow, FIG. 22A). The frequency of protein delivery into cells within metastases was three-fold higher in induced EBV-003 versus uninduced EBV-003 (**, P<0.01; FIG. 22B). Taken together, these results indicate that induction of flhDC improves protein delivery in both primary and metastatic breast tumors.

All publications, patents, and patent applications mentioned in this specification are herein incorporated by reference to the same extent as if each individual publication, patent, or patent application was specifically and individually indicated to be incorporated by reference. In the event that the definition of a term incorporated by reference conflicts with a term defined herein, this specification shall control.

1. A bacterial cell comprising:
a) a SseJ deletion or wherein expression of SseJ has been reduced; and
b) a lysis gene or lysis cassette operably linked to an intracellularly induced Salmonella promoter.

2. The cell of claim 1, wherein the bacterial cell is an intratumoral bacteria cell.

3. The cell of claim 1, wherein the bacterial cell is a Clostridium, Escherichia coli Bifidus or Salmonella cell.

4. The cell of claim 1, wherein the bacterial cell is a Salmonella cell.

5. The cell of claim 1, wherein the lysis cassette is Lysin E from phage phiX174, the lysis cassette of phage iEPS5, or the lysis cassette from lambda phage.

6. The cell of claim 1, wherein the intracellularly induced Salmonella promoter is a promoter for one of the genes in Salmonella pathogenicity island 2 type III secretion system (SPI2-T3SS) selected from the group SpiC/SsaB, SseF, SseG, SseI, SseJ, SseKJ, SseK2, SifA, SifB, PipB, PipB2, SopD2, GogB, SseL, SteC, SspH1, SspH2, or SirP.

7. The cell of claim 1, wherein the cell does not comprise endogenous flhDC expression.

8. The cell of claim 1, wherein the cell does not comprise endogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL expression.

9. The cell claim 1, wherein the cell comprises an exogenous inducible promoter operably linked to an endogenous or exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene.

10. The cell of claim 9, wherein the exogenous inducible promoter is operably linked to the endogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene.

11. The cell of claim 9, wherein the exogenous inducible promoter is operably linked the exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgI, flgJ, flgK and/or flgL gene.

12. The cell of claim 9, wherein the exogenous inducible promoter comprises the arabinose inducible promoter PBAD (L-arabinose), LacI (IPTG), salR or nahR (acetyl salicylic acid (ASA)).

13. The cell of claim 1, where the cells comprise a plasmid that expresses a peptide.

14. The cell of claim 13, wherein the peptide is a therapeutic peptide.

15. The cell of claim 13, wherein the peptide is NIPP1 or activated caspase 3.

16. A composition comprising a population of cells of claim 1 and a pharmaceutically acceptable carrier.

17. A method to colonize a tumor and/or tumor associated cells comprising administering a population of the bacterial cells of claim 1 to a subject in need thereof.

18. The method of claim 17, wherein the tumor associated cells are intratumoral immune cells or stromal cells within tumors.

19. A method to treat cancer comprising administering to subject in need thereof an effective amount of a population of the bacterial cells of claim 1 so as to treat said cancer.

20. A method of inhibiting tumor growth/proliferation or reducing the volume/size of a tumor comprising administering to subject in need thereof an effective amount of a population of the bacterial cells of claim 1, so as to suppress tumor growth or reduce the volume of the tumor.

21. A method to treat, reduce formation/number or inhibit spread of metastases comprising administering to subject in need thereof an effective amount of a population of the bacterial cells of claim 1, so as to treat, reduce formation/number or inhibit spread of metastases.

22. The method of claim 17, wherein the tumor, tumor associated cells, cancer, or metastases are a lung, liver, kidney, breast, prostate, pancreatic, skin, colon, head and neck, ovarian and/or gastroenterological tumor, tumor associated cells, cancer or metastases.

23. The method of claim 17, wherein the bacterial cells deliver a therapeutic peptide, such as NIPP1 or activated caspase 3, to said tumor, tumor associated cells, cancer or metastases.

24. The method of claim 17, wherein endogenous expression of flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgL, flgJ, flgK and/or flgL is under control of an exogenous inducible promoter.

25. The method of claim 17, wherein expression of flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgL, flgJ, flgK and/or flgL is under the control of an inducible promoter, wherein the bacterial cells comprise an exogenous inducible promoter operably linked an exogenous flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgL, flgJ, flgK and/or flgL gene.

26. The method of claim 24, wherein the expression of flhDC, motA, motB, flhE, cheZ, cheY cheB, cheR, cheM, cheW, cheA, fliA, fliY, fliZ, fliB, fliS, fliE, fliF, fliJ, fliL, fliM, fliN, fliO, flip, fliQ, fliR, fliG, fliH, fliI, fliT, fliD, fliC, fljB, ycrG, flgN, flgM, flgA, flgB, flgC, flgD, flgE, flgF, flgG, flgH, flgL, flgJ, flgK and/or flgL is induced after said tumor, tumor associated cells, cancer or metastases have been colonized by said bacteria.
